(19) United States
(12) Patent Application Publication
(10) Pub. No.: US 2025/0066376 A1
(43) Pub. Date: Feb. 27, 2025

(54) OPIOID RECEPTOR ANTAGONIST PRODRUGS

(71) Applicant: Nirsum Laboratories, Inc., New York, NY (US)

(72) Inventor: Nikej N. Shah, New York, NY (US)

(21) Appl. No.: 18/893,561

(22) Filed: Sep. 23, 2024

(51) Int. Cl. C07D 489/08; A61K 31/485; C07D 221/28

(52) U.S. Cl. CPC C07D 489/08; A61K 31/485; C07D 221/28

(57) ABSTRACT

Provided herein are prodrugs of opioid receptor antagonists such as samidorphan, methocinnamox, and naloxone, pharmaceutical compositions comprising said compounds, and methods for using said compounds for the treatment of diseases and disorders such as a depressive disorder, alcohol dependence and opioid dependence.

### BACKGROUND

A need exists in the medicinal arts for compositions and methods for the modulation of opioid receptor activity in the course of treating diseases and disorders such as a depressive disorder, alcohol dependence, opioid dependence, and side effects brought by opioid use.

### SUMMARY OF THE INVENTION

This invention is directed to prodrug compounds of opioid receptor antagonists such as samidorphan, methocinnamox, and naloxone that are encompassed within Formula (I), (II) or (III) (collectively referred to herein as the “Disclosed Compounds”), pharmaceutical compositions comprising said compounds, and methods for using said compounds for the treatment of diseases and disorders such as a depressive disorder, alcohol dependence, opioid dependence, and side effects brought by opioid use.

In one aspect, provided herein is a compound represented by the following structural formula:

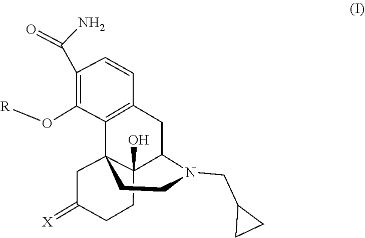

or a pharmaceutically acceptable salt thereof. The definition of each variable is provided below.

In another aspect, provided herein is a compound represented by the following structural formula:

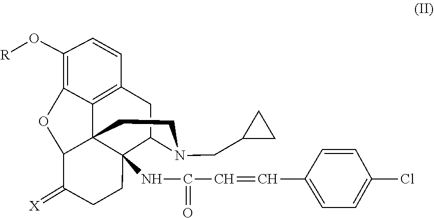

or a pharmaceutically acceptable salt thereof. The definition of each variable is provided below.

In yet another aspect, provided herein is a compound represented by the following structural formula:

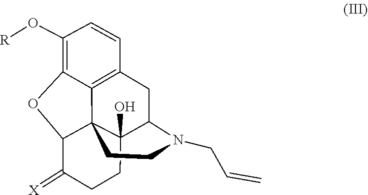

or a pharmaceutically acceptable salt thereof. The definition of each variable is provided below.

Also provided is a pharmaceutical composition comprising a pharmaceutically acceptable excipient and a Disclosed Compound, or a pharmaceutically acceptable salt thereof.

Also provided is a method of treating opioid dependence, alcohol dependence, a depressive disorder, itchiness and opioid-induced constipation brought by opioid use, or congenital insensitivity to pain with anhidrosis, in a patient in need thereof comprising administering a pharmaceutical composition comprising a Disclosed Compound, or pharmaceutically acceptable salt thereof, and a pharmaceutically acceptable excipient.

Also provided is a method of increasing blood pressure in a patient experiencing septic shock comprising administering a pharmaceutical composition comprising a Disclosed Compound, or pharmaceutically acceptable salt thereof, and a pharmaceutically acceptable excipient.

### DETAILED DESCRIPTION

### Opioid Receptor Antagonists Prodrugs

Provided herein are prodrugs of opioid receptor antagonists such as samidorphan, methocinnamox, and naloxone.

Samidorphan is a μ-opioid receptor antagonist that has been investigated for the treatment of alcoholism and cocaine addiction. It is also under development for the treatment of major depressive disorder and possibly other psychiatric conditions. Samidorphan has the IUPAC name 17-(cyclopropylmethyl)-4,14-dihydroxy-6-oxomorphinan-3-carboxamide and has the structure provided in Formula (A).

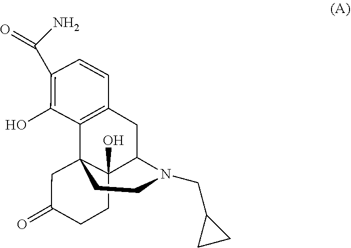

Methocinnamox is a μ-opioid receptor antagonist is under development for use in reversing opioid effects and for opioid overdose, with the structure provided in Formula (B).

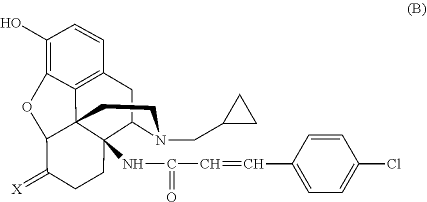

Naloxone is a non-selective and competitive opioid receptor antagonist. It works by reversing the depression of the central nervous system and respiratory system caused by opioids. Sold under the brand name Narcan among others, naloxone was patented in 1961 and approved for opioid overdose in the United States in 1971. Naloxone is useful both in acute opioid overdose and in reducing respiratory or mental depression due to opioids. It is used as an adjunctive agent in preventing opioid abuse. It can also be used to treat side effects brought by opioid use, including itchiness and opioid-induced constipation. Naloxone is also experimentally used in the treatment for congenital insensitivity to pain with anhidrosis, an extremely rare disorder that renders one unable to feel pain or differentiate temperatures. Naloxone may be useful as an adjunctive agent to increase blood pressure in the management of septic shock. Naloxone has the IUPAC name 17-allyl-4,5α-epoxy-3,14-dihydroxymorphinan-6-one and has the structure provided in Formula (C).

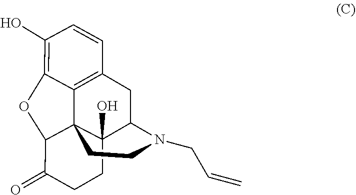

Although there is a great potential for using samidorphan, methocinnamox, and naloxone in the treatment of a depressive disorder, alcohol dependence and opioid dependence, among others, the problem with these drugs is that they have very short period of action. One of the solutions to overcome the problem of short period of action of samidorphan, methocinnamox, and naloxone is to use prodrugs which provide a long, sustained, and controlled release of samidorphan, methocinnamox, and naloxone opioid receptor antagonists upon administration into the body.

In a 1^{st} aspect, provided herein is a compound, or pharmaceutically acceptable salt thereof, having a structure provided in Formula (I):

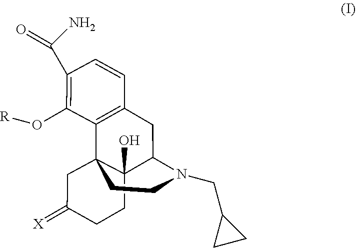

wherein:
X is O or CH₂;
R is selected from the group consisting of:
   (a) —C(O)R¹,
   (b) —C(O)OR¹,
   (c) —CH₂OC(O)R¹,
   (d) —CH₂OC(O)OR^{1′} and
   (e) —CH₂OR¹;
each instance of R¹ is independently —(CH₂)_{q}CH₃,

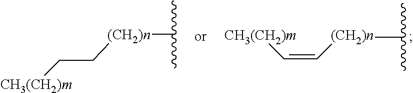

m and n are integers between 0 and 23, and the sum of m and n is an integer between 6 and 23; and
q is an integer between 0 and 25.

In a 2^{nd} aspect, provided herein is a compound, or pharmaceutically acceptable salt thereof, having a structure provided in Formula (I-1),

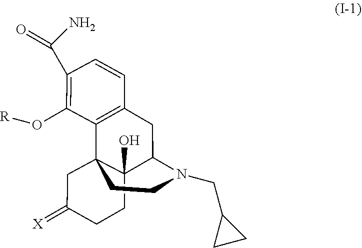

wherein:
X is O or CH₂;
R is —C(O)R¹;
wherein R¹ is —(CH₂)_{q}CH₃; and
wherein q is an integer between 0 and 25.

In one specific embodiment, q is 0 and X is O.

In another specific embodiment, q is 0 and X is CH₂.

In yet another specific embodiment, q is 1 and X is O.

In yet another specific embodiment, q is 1 and X is CH₂.

In yet another specific embodiment, q is 2 and X is O.

In yet another specific embodiment, q is 2 and X is CH₂.

In yet another specific embodiment, q is 3 and X is O.

In yet another specific embodiment, q is 3 and X is CH₂.

In yet another specific embodiment, q is 4 and X is O.

In yet another specific embodiment, q is 4 and X is CH₂.

In yet another specific embodiment, q is 5 and X is O.

In yet another specific embodiment, q is 5 and X is CH₂.

In yet another specific embodiment, q is 6 and X is O.

In yet another specific embodiment, q is 6 and X is CH₂.

In yet another specific embodiment, q is 7 and X is O.

In yet another specific embodiment, q is 7 and X is CH₂.

In yet another specific embodiment, q is 8 and X is O.

In yet another specific embodiment, q is 8 and X is CH₂.

In yet another specific embodiment, q is 9 and X is O.

In yet another specific embodiment, q is 9 and X is CH₂.

In yet another specific embodiment, q is 10 and X is O.

In yet another specific embodiment, q is 10 and X is CH₂.

In yet another specific embodiment, q is 11 and X is O.

In yet another specific embodiment, q is 11 and X is CH₂.

In yet another specific embodiment, q is 12 and X is O.

In yet another specific embodiment, q is 12 and X is CH₂.

In yet another specific embodiment, q is 13 and X is O.

In yet another specific embodiment, q is 13 and X is CH₂.

In yet another specific embodiment, q is 14 and X is O.

In yet another specific embodiment, q is 14 and X is CH₂.

In yet another specific embodiment, q is 15 and X is O.

In yet another specific embodiment, q is 15 and X is CH₂.

In yet another specific embodiment, q is 16 and X is O.

In yet another specific embodiment, q is 16 and X is CH₂.

In yet another specific embodiment, q is 17 and X is O.

In yet another specific embodiment, q is 17 and X is CH₂.

In yet another specific embodiment, q is 18 and X is O.

In yet another specific embodiment, q is 18 and X is CH₂.

In yet another specific embodiment, q is 19 and X is O.

In yet another specific embodiment, q is 19 and X is CH₂.

In yet another specific embodiment, q is 20 and X is O.

In yet another specific embodiment, q is 20 and X is CH₂.

In yet another specific embodiment, q is 21 and X is O.

In yet another specific embodiment, q is 21 and X is CH₂.

In yet another specific embodiment, q is 22 and X is O.

In yet another specific embodiment, q is 22 and X is CH₂.

In yet another specific embodiment, q is 23 and X is O.

In yet another specific embodiment, q is 23 and X is CH₂.

In yet another specific embodiment, q is 24 and X is O.

In yet another specific embodiment, q is 24 and X is CH₂.

In yet another specific embodiment, q is 25 and X is O.

In yet another specific embodiment, q is 25 and X is CH₂.

Pharmaceutically acceptable salts of any one of the foregoing embodiments described in the 2^{nd} aspect are also included.

In a 3^{rd} aspect, provided herein is a compound, or pharmaceutically acceptable salt thereof, having a structure provided in Formula (I-2),

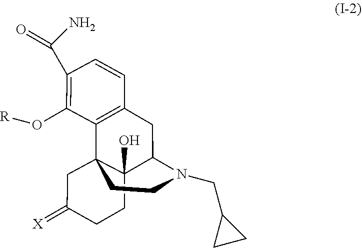

wherein:
X is O or CH₂;
R is —C(O)OR¹;
wherein R¹ is —(CH₂)_{q}CH₃; and
wherein q is an integer between 0 and 25.

In one specific embodiment, q is 0 and X is O.

In another specific embodiment, q is 0 and X is CH₂.

In yet another specific embodiment, q is 1 and X is O.

In yet another specific embodiment, q is 1 and X is CH₂.

In yet another specific embodiment, q is 2 and X is O.

In yet another specific embodiment, q is 2 and X is CH₂.

In yet another specific embodiment, q is 3 and X is O.

In yet another specific embodiment, q is 3 and X is CH₂.

In yet another specific embodiment, q is 4 and X is O.

In yet another specific embodiment, q is 4 and X is CH₂.

In yet another specific embodiment, q is 5 and X is O.

In yet another specific embodiment, q is 5 and X is CH₂.

In yet another specific embodiment, q is 6 and X is O.

In yet another specific embodiment, q is 6 and X is CH₂.

In yet another specific embodiment, q is 7 and X is O.

In yet another specific embodiment, q is 7 and X is CH₂.

In yet another specific embodiment, q is 8 and X is O.

In yet another specific embodiment, q is 8 and X is CH₂.

In yet another specific embodiment, q is 9 and X is O.

In yet another specific embodiment, q is 9 and X is CH₂.

In yet another specific embodiment, q is 10 and X is O.

In yet another specific embodiment, q is 10 and X is CH₂.

In yet another specific embodiment, q is 11 and X is O.

In yet another specific embodiment, q is 11 and X is CH₂.

In yet another specific embodiment, q is 12 and X is O.

In yet another specific embodiment, q is 12 and X is CH₂.

In yet another specific embodiment, q is 13 and X is O.

In yet another specific embodiment, q is 13 and X is CH₂.

In yet another specific embodiment, q is 14 and X is O.

In yet another specific embodiment, q is 14 and X is CH₂.

In yet another specific embodiment, q is 15 and X is O.

In yet another specific embodiment, q is 15 and X is CH₂.

In yet another specific embodiment, q is 16 and X is O.

In yet another specific embodiment, q is 16 and X is CH₂.

In yet another specific embodiment, q is 17 and X is O.

In yet another specific embodiment, q is 17 and X is CH₂.

In yet another specific embodiment, q is 18 and X is O.

In yet another specific embodiment, q is 18 and X is CH₂.

In yet another specific embodiment, q is 19 and X is O.

In yet another specific embodiment, q is 19 and X is CH₂.

In yet another specific embodiment, q is 20 and X is O.

In yet another specific embodiment, q is 20 and X is CH₂.

In yet another specific embodiment, q is 21 and X is O.

In yet another specific embodiment, q is 21 and X is CH₂.

In yet another specific embodiment, q is 22 and X is O.

In yet another specific embodiment, q is 22 and X is CH₂.

In yet another specific embodiment, q is 23 and X is O.

In yet another specific embodiment, q is 23 and X is CH₂.

In yet another specific embodiment, q is 24 and X is O.

In yet another specific embodiment, q is 24 and X is CH₂.

In yet another specific embodiment, q is 25 and X is O.

In yet another specific embodiment, q is 25 and X is CH₂.

Pharmaceutically acceptable salts of any one of the foregoing embodiments described in the 3^{rd} aspect are also included.

In a 4^{th} aspect, provided herein is a compound, or pharmaceutically acceptable salt thereof, having a structure provided in Formula (I-3),

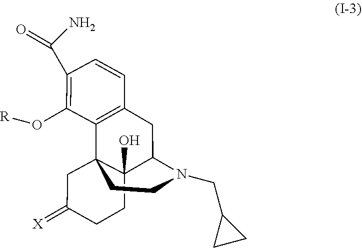

wherein:
X is O or CH₂;
R is —CH₂OC(O)R¹;
wherein R¹ is —(CH₂)_{q}CH₃; and
wherein q is an integer between 0 and 25.

In one specific embodiment, q is 0 and X is O.

In another specific embodiment, q is 0 and X is CH₂.

In yet another specific embodiment, q is 1 and X is O.

In yet another specific embodiment, q is 1 and X is CH₂.

In yet another specific embodiment, q is 2 and X is O.

In yet another specific embodiment, q is 2 and X is CH₂.

In yet another specific embodiment, q is 3 and X is O.

In yet another specific embodiment, q is 3 and X is CH₂.

In yet another specific embodiment, q is 4 and X is O.

In yet another specific embodiment, q is 4 and X is CH₂.

In yet another specific embodiment, q is 5 and X is O.

In yet another specific embodiment, q is 5 and X is CH₂.

In yet another specific embodiment, q is 6 and X is O.

In yet another specific embodiment, q is 6 and X is CH₂.

In yet another specific embodiment, q is 7 and X is O.

In yet another specific embodiment, q is 7 and X is CH₂.

In yet another specific embodiment, q is 8 and X is O.

In yet another specific embodiment, q is 8 and X is CH₂.

In yet another specific embodiment, q is 9 and X is O.

In yet another specific embodiment, q is 9 and X is CH₂.

In yet another specific embodiment, q is 10 and X is O.

In yet another specific embodiment, q is 10 and X is CH₂.

In yet another specific embodiment, q is 11 and X is O.

In yet another specific embodiment, q is 11 and X is CH₂.

In yet another specific embodiment, q is 12 and X is O.

In yet another specific embodiment, q is 12 and X is CH₂.

In yet another specific embodiment, q is 13 and X is O.

In yet another specific embodiment, q is 13 and X is CH₂.

In yet another specific embodiment, q is 14 and X is O.

In yet another specific embodiment, q is 14 and X is CH₂.

In yet another specific embodiment, q is 15 and X is O.

In yet another specific embodiment, q is 15 and X is CH₂.

In yet another specific embodiment, q is 16 and X is O.

In yet another specific embodiment, q is 16 and X is CH₂.

In yet another specific embodiment, q is 17 and X is O.

In yet another specific embodiment, q is 17 and X is CH₂.

In yet another specific embodiment, q is 18 and X is O.

In yet another specific embodiment, q is 18 and X is CH₂.

In yet another specific embodiment, q is 19 and X is O.

In yet another specific embodiment, q is 19 and X is CH₂.

In yet another specific embodiment, q is 20 and X is O.

In yet another specific embodiment, q is 20 and X is CH₂.

In yet another specific embodiment, q is 21 and X is O.

In yet another specific embodiment, q is 21 and X is CH₂.

In yet another specific embodiment, q is 22 and X is O.

In yet another specific embodiment, q is 22 and X is CH₂.

In yet another specific embodiment, q is 23 and X is O.

In yet another specific embodiment, q is 23 and X is CH₂.

In yet another specific embodiment, q is 24 and X is O.

In yet another specific embodiment, q is 24 and X is CH₂.

In yet another specific embodiment, q is 25 and X is O.

In yet another specific embodiment, q is 25 and X is CH₂.

Pharmaceutically acceptable salts of any one of the foregoing embodiments described in the 4^{th} aspect are also included.

In a 5^{th} aspect, provided herein is a compound, or pharmaceutically acceptable salt thereof, having a structure provided in Formula (I-4),

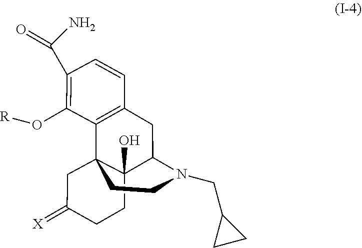

wherein:
X is O or CH₂;
R is —CH₂OC(O)OR¹;
wherein R¹ is —(CH₂)_{q}CH₃; and
wherein q is an integer between 0 and 25.

In one specific embodiment, q is 0 and X is O.

In another specific embodiment, q is 0 and X is CH₂.

In yet another specific embodiment, q is 1 and X is O.

In yet another specific embodiment, q is 1 and X is CH₂.

In yet another specific embodiment, q is 2 and X is O.

In yet another specific embodiment, q is 2 and X is CH₂.

In yet another specific embodiment, q is 3 and X is O.

In yet another specific embodiment, q is 3 and X is CH₂.

In yet another specific embodiment, q is 4 and X is O.

In yet another specific embodiment, q is 4 and X is CH₂.

In yet another specific embodiment, q is 5 and X is O.

In yet another specific embodiment, q is 5 and X is CH₂.

In yet another specific embodiment, q is 6 and X is O.

In yet another specific embodiment, q is 6 and X is CH₂.

In yet another specific embodiment, q is 7 and X is O.

In yet another specific embodiment, q is 7 and X is CH₂.

In yet another specific embodiment, q is 8 and X is O.

In yet another specific embodiment, q is 8 and X is CH₂.

In yet another specific embodiment, q is 9 and X is O.

In yet another specific embodiment, q is 9 and X is CH₂.

In yet another specific embodiment, q is 10 and X is O.

In yet another specific embodiment, q is 10 and X is CH₂.

In yet another specific embodiment, q is 11 and X is O.

In yet another specific embodiment, q is 11 and X is CH₂.

In yet another specific embodiment, q is 12 and X is O.

In yet another specific embodiment, q is 12 and X is CH₂.

In yet another specific embodiment, q is 13 and X is O.

In yet another specific embodiment, q is 13 and X is CH₂.

In yet another specific embodiment, q is 14 and X is O.

In yet another specific embodiment, q is 14 and X is CH₂.

In yet another specific embodiment, q is 15 and X is O.

In yet another specific embodiment, q is 15 and X is CH₂.

In yet another specific embodiment, q is 16 and X is O.

In yet another specific embodiment, q is 16 and X is CH₂.

In yet another specific embodiment, q is 17 and X is O.

In yet another specific embodiment, q is 17 and X is CH₂.

In yet another specific embodiment, q is 18 and X is O.

In yet another specific embodiment, q is 18 and X is CH₂.

In yet another specific embodiment, q is 19 and X is O.

In yet another specific embodiment, q is 19 and X is CH₂.

In yet another specific embodiment, q is 20 and X is O.

In yet another specific embodiment, q is 20 and X is CH₂.

In yet another specific embodiment, q is 21 and X is O.

In yet another specific embodiment, q is 21 and X is CH₂.

In yet another specific embodiment, q is 22 and X is O.

In yet another specific embodiment, q is 22 and X is CH₂.

In yet another specific embodiment, q is 23 and X is O.

In yet another specific embodiment, q is 23 and X is CH₂.

In yet another specific embodiment, q is 24 and X is O.

In yet another specific embodiment, q is 24 and X is CH₂.

In yet another specific embodiment, q is 25 and X is O.

In yet another specific embodiment, q is 25 and X is CH₂.

Pharmaceutically acceptable salts of any one of the foregoing embodiments described in the 5^{th} aspect are also included.

In a 6^{th} aspect, provided herein is a compound, or pharmaceutically acceptable salt thereof, having a structure provided in Formula (I-5),

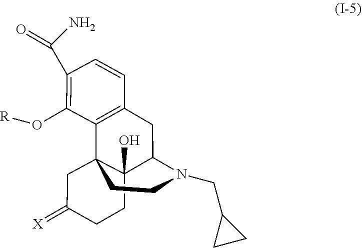

wherein:
X is O or CH₂;
R is —C(O)R¹;
wherein R¹ is

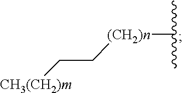

and
wherein m and n are integers between 0 and 23, and the sum of m and n is an integer between 6 and 23.

In one specific embodiment, m is 0, n is 23, and X is O.

In another specific embodiment, m is 0, n is 23, and X is CH₂.

In yet another specific embodiment, m is 1, n is 22, and X is O.

In yet another specific embodiment, m is 1, n is 22, and X is CH₂.

In yet another specific embodiment, m is 2, n is 21, and X and X is O.

In yet another specific embodiment, m is 2, n is 21, and X is CH₂.

In yet another specific embodiment, m is 3, n is 20, and X is O.

In yet another specific embodiment, m is 3, n is 20, and X is CH₂.

In yet another specific embodiment, m is 4, n is 19, and X is O.

In yet another specific embodiment, m is 4, n is 19, and X is CH₂.

In yet another specific embodiment, m is 5, n is 18, and X is O.

In yet another specific embodiment, m is 5, n is 18, and X is CH₂.

In yet another specific embodiment, m is 6, n is 17, and X is O.

In yet another specific embodiment, m is 6, n is 17, and X is CH₂.

In yet another specific embodiment, m is 7, n is 16, and X is O.

In yet another specific embodiment, m is 7, n is 16, and X is CH₂.

In yet another specific embodiment, m is 8, n is 15, and X is O.

In yet another specific embodiment, m is 8, n is 15, and X is CH₂.

In yet another specific embodiment, m is 9, n is 14, and X is O.

In yet another specific embodiment, m is 9, n is 14, and X is CH₂.

In yet another specific embodiment, m is 10, n is 13, and X is O.

In yet another specific embodiment, m is 10, n is 13, and X is CH₂.

In yet another specific embodiment, m is 11, n is 12, and X is O.

In yet another specific embodiment, m is 11, n is 12, and X is CH₂.

In yet another specific embodiment, m is 12, n is 11, and X is O.

In yet another specific embodiment, m is 12, n is 11, and X is CH₂.

In yet another specific embodiment, m is 13, n is 10, and X is O.

In yet another specific embodiment, m is 13, n is 10, and X is CH₂.

In yet another specific embodiment, m is 14, n is 9, and X is O.

In yet another specific embodiment, m is 14, n is 9, and X is CH₂.

In yet another specific embodiment, m is 15, n is 8, and X is O.

In yet another specific embodiment, m is 15, n is 8, and X is CH₂.

In yet another specific embodiment, m is 16, n is 7, and X is O.

In yet another specific embodiment, m is 16, n is 7, and X is CH₂.

In yet another specific embodiment, m is 17, n is 6, and X is O.

In yet another specific embodiment, m is 17, n is 6, and X is CH₂.

In yet another specific embodiment, m is 18, n is 5, and X is O.

In yet another specific embodiment, m is 18, n is 5, and X is CH₂.

In yet another specific embodiment, m is 19, n is 4, and X is O.

In yet another specific embodiment, m is 19, n is 4, and X is CH₂.

In yet another specific embodiment, m is 20, n is 3, and X is O.

In yet another specific embodiment, m is 20, n is 3, and X is CH₂.

In yet another specific embodiment, m is 21, n is 2, and X is O.

In yet another specific embodiment, m is 21, n is 2, and X is CH₂.

In yet another specific embodiment, m is 22, n is 1, and X is O.

In yet another specific embodiment, m is 22, n is 1, and X is CH₂.

In yet another specific embodiment, m is 23, n is 0, and X is O.

In yet another specific embodiment, m is 23, n is 0, and X is CH₂.

In a First Specific Embodiment for Formula (I-5), X is CH₂ and; m is 0 and n is 22; m is 1 and n is 21; m is 2 and n is 20; m is 3 and n is 19; m is 4 and n is 18; m is 5, n is 17; m is 6 and n is 16; m is 7 and n is 15; m is 8 and n is 14; m is 9 and n is 13; m is 10 and n is 12; m is 11 and n is 11; m is 12 and n is 10; m is 13 and n is 9; m is 14 and n is 8; m is 15 and n is 7; m is 16 and n is 6; m is 17 and n is 5; m is 18 and n is 4; m is 19 and n is 3; m is 20 and n is 2; m is 21 and n is 1; m is 22 and n is 0; m is 0 and n is 21; m is 1 and n is 20; m is 2 and n is 19; m is 3 and n is 18; m is 4 and n is 17; m is 5, n is 16; m is 6 and n is 15; m is 7 and n is 14; m is 8 and n is 13; m is 9 and n is 12; m is 10 and n is 11; m is 11 and n is 10; m is 12 and n is 9; m is 13 and n is 8; m is 14 and n is 7; m is 15 and n is 6; m is 16 and n is 5; m is 17 and n is 4; m is 18 and n is 3; m is 19 and n is 2; m is 20 and n is 1; m is 21 and n is 0; m is 0 and n is 20; m is 1 and n is 19; m is 2 and n is 18; m is 3 and n is 17; m is 4 and n is 16; m is 5, n is 15; m is 6 and n is 14; m is 7 and n is 13; m is 8 and n is 12; m is 9 and n is 11; m is 10 and n is 10; m is 11 and n is 9; m is 12 and n is 8; m is 13 and n is 7; m is 14 and n is 6; m is 15 and n is 5; m is 16 and n is 4; m is 17 and n is 3; m is 18 and n is 2; m is 19 and n is 1; m is 20 and n is 0; m is 0 and n is 19; m is 1 and n is 18; m is 2 and n is 17; m is 3 and n is 16; m is 4 and n is 15; m is 5, n is 14; m is 6 and n is 13; m is 7 and n is 12; m is 8 and n is 11; m is 9 and n is 10; m is 10 and n is 9; m is 11 and n is 8; m is 12 and n is 7; m is 13 and n is 6; m is 14 and n is 5; m is 15 and n is 4; m is 16 and n is 3; m is 17 and n is 2; m is 18 and n is 1; m is 19 and n is 0; m is 0 and n is 18; m is 1 and n is 17; m is 2 and n is 16; m is 3 and n is 15; m is 4 and n is 14; m is 5, n is 13; m is 6 and n is 12; m is 7 and n is 11; m is 8 and n is 10; m is 9 and n is 9; m is 10 and n is 8; m is 11 and n is 7; m is 12 and n is 6; m is 13 and n is 5; m is 14 and n is 4; m is 15 and n is 3; m is 16 and n is 2; m is 17 and n is 1; m is 18 and n is 0; m is 0 and n is 17; m is 1 and n is 16; m is 2 and n is 15; m is 3 and n is 14; m is 4 and n is 13; m is 5, n is 12; m is 6 and n is 11; m is 7 and n is 10; m is 8 and n is 9; m is 9 and n is 8; m is 10 and n is 7; m is 11 and n is 6; m is 12 and n is 5; m is 13 and n is 4; m is 14 and n is 3; m is 15 and n is 2; m is 16 and n is 1; m is 17 and n is 0; m is 0 and n is 16; m is 1 and n is 15; m is 2 and n is 14; m is 3 and n is 13; m is 4 and n is 12; m is 5, n is 11; m is 6 and n is 10; m is 7 and n is 9; m is 8 and n is 8; m is 9 and n is 7; m is 10 and n is 6; m is 11 and n is 5; m is 12 and n is 4; m is 13 and n is 3; m is 14 and n is 2; m is 15 and n is 1; m is 16 and n is 0; m is 0 and n is 15; m is 1 and n is 14; m is 2 and n is 13; m is 3 and n is 12; m is 4 and n is 11; m is 5, n is 10; m is 6 and n is 9; m is 7 and n is 8; m is 8 and n is 7; m is 9 and n is 6; m is 10 and n is 5; m is 11 and n is 4; m is 12 and n is 3; m is 13 and n is 2; m is 14 and n is 1; m is 15 and n is 0; m is 0 and n is 14; m is 1 and n is 13; m is 2 and n is 12; m is 3 and n is 11; m is 4 and n is 10; m is 5, n is 9; m is 6 and n is 8; m is 7 and n is 7; m is 8 and n is 6; m is 9 and n is 5; m is 10 and n is 4; m is 11 and n is 3; m is 12 and n is 2; m is 13 and n is 1; m is 14 and n is 0; m is 0 and n is 13; m is 1 and n is 12; m is 2 and n is 11; m is 3 and n is 10; m is 4 and n is 9; m is 5, n is 8; m is 6 and n is 7; m is 7 and n is 6; m is 8 and n is 5; m is 9 and n is 4; m is 10 and n is 3; m is 11 and n is 2; m is 12 and n is 1; m is 13 and n is 0; m is 0 and n is 12; m is 1 and n is 11; m is 2 and n is 10; m is 3 and n is 9; m is 4 and n is 8; m is 5, n is 7; m is 6 and n is 6; m is 7 and n is 5; m is 8 and n is 4; m is 9 and n is 3; m is 10 and n is 2; m is 11 and n is 1; m is 12 and n is 0; m is 0 and n is 11; m is 1 and n is 10; m is 2 and n is 9; m is 3 and n is 8; m is 4 and n is 7; m is 5, n is 6; m is 6 and n is 5; m is 7 and n is 4; m is 8 and n is 3; m is 9 and n is 2; m is 10 and n is 1; m is 11 and n is 0; m is 0 and n is 10; m is 1 and n is 9; m is 2 and n is 8; m is 3 and n is 7; m is 4 and n is 6; m is 5, n is 5; m is 6 and n is 4; m is 7 and n is 3; m is 8 and n is 2; m is 9 and n is 1; m is 10 and n is 0; m is 0 and n is 9; m is 1 and n is 8; m is 2 and n is 7; m is 3 and n is 6; m is 4 and n is 5; m is 5, n is 4; m is 6 and n is 3; m is 7 and n is 2; m is 8 and n is 1; m is 9 and n is 0; m is 0 and n is 8; m is 1 and n is 7; m is 2 and n is 6; m is 3 and n is 5; m is 4 and n is 4; m is 5, n is 3; m is 6 and n is 2; m is 7 and n is 1; m is 8 and n is 0; m is 0 and n is 7; m is 1 and n is 6; m is 2 and n is 5; m is 3 and n is 4; m is 4 and n is 3; m is 5, n is 2; m is 6 and n is 1; m is 7 and n is 0; m is 0 and n is 6; m is 1 and n is 5; m is 2 and n is 4; m is 3 and n is 4; m is 4 and n is 2; m is 5, n is 1; or m is 6 and n is 0; and R and R′ are as described above for Formula (I-5).

In a Second Specific Embodiment for Formula (I-5), R and R¹ are as described above for Formula (I-5), X is 0, and n and m are as described in the First Specific Embodiment.

In a Third Specific Embodiment for Formula (I-5), R and R¹, X, n and m are as described for the First Specific Embodiment for Formula (I-5), provided that one CH₂CH₂ group is replaced with CH═CH, e.g., cis CH═CH. In one embodiment the two double bonds are separated by a CH₂.

In a Fourth Specific Embodiment for Formula (I-5), R and R′, X, n and m are as described for the Second Specific Embodiment for Formula (I-5), provided that one CH₂CH₂ group is replaced with CH═CH, e.g., CH═CH cis. In one embodiment the two double bonds are separated by a CH₂.

In a Fifth Specific Embodiment for Formula (I-5), R and R¹, X, n and m are as described for the First Specific Embodiment for Formula (I-5), provided that two CH₂CH₂ groups are replaced with CH═CH, e.g., CH═CH cis. In one embodiment all double bonds are separated by CH₂, i.e., CH═CHCH₂CH═CHCH₂CH═CH.

In a Sixth Specific Embodiment for Formula (I-5), R and R¹, X, n and m are as described for the Second Specific Embodiment for Formula (I-5), provided that two CH₂CH₂ groups are replaced with CH═CH, e.g., CH═CH cis. In one embodiment all double bonds are separated by CH₂, i.e., CH═CHCH₂CH═CHCH₂CH═CH.

Pharmaceutically acceptable salts of any one of the foregoing embodiments described in the 6^{th} aspect are also included.

In a 7^{th} aspect, provided herein is a compound, or pharmaceutically acceptable salt thereof, having a structure provided in Formula (I-6),

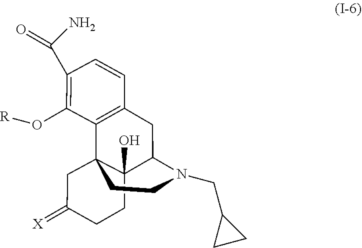

wherein:
X is O or CH₂;
R is —C(O)R¹;
wherein R¹ is

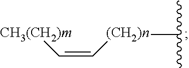

and
wherein m and n are integers between 0 and 23, and the sum of m and n is an integer between 6 and 23.

In one specific embodiment, m is 0, n is 23, and X is O.

In another specific embodiment, m is 0, n is 23, and X is CH₂.

In yet another specific embodiment, m is 1, n is 22, and X is O.

In yet another specific embodiment, m is 1, n is 22, and X is CH₂.

In yet another specific embodiment, m is 2, n is 21, and X is O.

In yet another specific embodiment, m is 2, n is 21, and X is CH₂.

In yet another specific embodiment, m is 3, n is 20, and X is O.

In yet another specific embodiment, m is 3, n is 20, and X is CH₂.

In yet another specific embodiment, m is 4, n is 19, and X is O.

In yet another specific embodiment, m is 4, n is 19, and X is CH₂.

In yet another specific embodiment, m is 5, n is 18, and X is O.

In yet another specific embodiment, m is 5, n is 18, and X is CH₂.

In yet another specific embodiment, m is 6, n is 17, and X is O.

In yet another specific embodiment, m is 6, n is 17, and X is CH₂.

In yet another specific embodiment, m is 7, n is 16, and X is O.

In yet another specific embodiment, m is 7, n is 16, and X is CH₂.

In yet another specific embodiment, m is 8, n is 15, and X is O.

In yet another specific embodiment, m is 8, n is 15, and X is CH₂.

In yet another specific embodiment, m is 9, n is 14, and X is O.

In yet another specific embodiment, m is 9, n is 14, and X is CH₂.

In yet another specific embodiment, m is 10, n is 13, and X is O.

In yet another specific embodiment, m is 10, n is 13, and X is CH₂.

In yet another specific embodiment, m is 11, n is 12, and X is O.

In yet another specific embodiment, m is 11, n is 12, and X is CH₂.

In yet another specific embodiment, m is 12, n is 11, and X is O.

In yet another specific embodiment, m is 12, n is 11, and X is CH₂.

In yet another specific embodiment, m is 13, n is 10, and X is O.

In yet another specific embodiment, m is 13, n is 10, and X is CH₂.

In yet another specific embodiment, m is 14, n is 9, and X is O.

In yet another specific embodiment, m is 14, n is 9, and X is CH₂.

In yet another specific embodiment, m is 15, n is 8, and X is O.

In yet another specific embodiment, m is 15, n is 8, and X is CH₂.

In yet another specific embodiment, m is 16, n is 7, and X is O.

In yet another specific embodiment, m is 16, n is 7, and X is CH₂.

In yet another specific embodiment, m is 17, n is 6, and X is O.

In yet another specific embodiment, m is 17, n is 6, and X is CH₂.

In yet another specific embodiment, m is 18, n is 5, and X is O.

In yet another specific embodiment, m is 18, n is 5, and X is CH₂.

In yet another specific embodiment, m is 19, n is 4, and X is O.

In yet another specific embodiment, m is 19, n is 4, and X is CH₂.

In yet another specific embodiment, m is 20, n is 3, and X is O.

In yet another specific embodiment, m is 20, n is 3, and X is CH₂.

In yet another specific embodiment, m is 21, n is 2, and X is O.

In yet another specific embodiment, m is 21, n is 2, and X is CH₂.

In yet another specific embodiment, m is 22, n is 1, and X is O.

In yet another specific embodiment, m is 22, n is 1, and X is CH₂.

In yet another specific embodiment, m is 23, n is 0, and X is O.

In yet another specific embodiment, m is 23, n is 0, and X is CH₂.

In another specific aspect, X, m and n in Formula (I-6) are as described in the First Specific Embodiment for Formula (I-5); and R and R¹ are as described above for Formula (I-6).

In another specific aspect, X, m and n in Formula (I-6) are as described in the Second Specific Embodiment for Formula (I-5); and R and R¹ are as described above for Formula (I-6).

In another specific aspect, X, m and n in Formula (I-6) are as described in the Third Specific Embodiment for Formula (I-5); and R and R¹ are as described above for Formula (I-6).

In another specific aspect, X, m and n in Formula (I-6) are as described in the Fourth Specific Embodiment for Formula (I-5); and R and R¹ are as described above for Formula (I-6).

In another specific aspect, X, m and n in Formula (I-6) are as described in the Fifth Specific Embodiment for Formula (I-5); and R and R¹ are as described above for Formula (I-6).

In another specific aspect, X, m and n in Formula (I-6) are as described in the Sixth Specific Embodiment for Formula (I-5); and R and R¹ are as described above for Formula (I-6).

Pharmaceutically acceptable salts of any one of the foregoing embodiments described in the 7^{th} aspect are also included.

In an 8^{th} aspect, provided herein is a compound, or pharmaceutically acceptable salt thereof, having a structure provided in Formula (I-7),

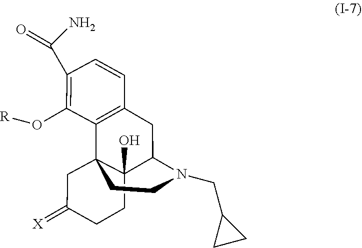

wherein:
X is O or CH₂;
R is —C(O)OR¹;
wherein R¹ is

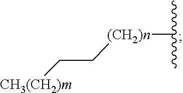

and
wherein m and n are integers between 0 and 23, and the sum of m and n is an integer between 6 and 23.

In one specific embodiment, m is 0, n is 23, and X is O.

In another specific embodiment, m is 0, n is 23, and X is CH₂.

In yet another specific embodiment, m is 1, n is 22, and X is O.

In yet another specific embodiment, m is 1, n is 22, and X is CH₂.

In yet another specific embodiment, m is 2, n is 21, and X is O.

In yet another specific embodiment, m is 2, n is 21, and X is CH₂.

In yet another specific embodiment, m is 3, n is 20, and X is O.

In yet another specific embodiment, m is 3, n is 20, and X is CH₂.

In yet another specific embodiment, m is 4, n is 19, and X is O.

In yet another specific embodiment, m is 4, n is 19, and X is CH₂.

In yet another specific embodiment, m is 5, n is 18, and X is O.

In yet another specific embodiment, m is 5, n is 18, and X is CH₂.

In yet another specific embodiment, m is 6, n is 17, and X is O.

In yet another specific embodiment, m is 6, n is 17, and X is CH₂.

In yet another specific embodiment, m is 7, n is 16, and X is O.

In yet another specific embodiment, m is 7, n is 16, and X is CH₂.

In yet another specific embodiment, m is 8, n is 15, and X is O.

In yet another specific embodiment, m is 8, n is 15, and X is CH₂.

In yet another specific embodiment, m is 9, n is 14, and X is O.

In yet another specific embodiment, m is 9, n is 14, and X is CH₂.

In yet another specific embodiment, m is 10, n is 13, and X is O.

In yet another specific embodiment, m is 10, n is 13, and X is CH₂.

In yet another specific embodiment, m is 11, n is 12, and X is O.

In yet another specific embodiment, m is 11, n is 12, and X is CH₂.

In yet another specific embodiment, m is 12, n is 11, and X is O.

In yet another specific embodiment, m is 12, n is 11, and X is CH₂.

In yet another specific embodiment, m is 13, n is 10, and X is O.

In yet another specific embodiment, m is 13, n is 10, and X is CH₂.

In yet another specific embodiment, m is 14, n is 9, and X is O.

In yet another specific embodiment, m is 14, n is 9, and X is CH₂.

In yet another specific embodiment, m is 15, n is 8, and X is O.

In yet another specific embodiment, m is 15, n is 8, and X is CH₂.

In yet another specific embodiment, m is 16, n is 7, and X is O.

In yet another specific embodiment, m is 16, n is 7, and X is CH₂.

In yet another specific embodiment, m is 17, n is 6, and X is O.

In yet another specific embodiment, m is 17, n is 6, and X is CH₂.

In yet another specific embodiment, m is 18, n is 5, and X is O.

In yet another specific embodiment, m is 18, n is 5, and X is CH₂.

In yet another specific embodiment, m is 19, n is 4, and X is O.

In yet another specific embodiment, m is 19, n is 4, and X is CH₂.

In yet another specific embodiment, m is 20, n is 3, and X is O.

In yet another specific embodiment, m is 20, n is 3, and X is CH₂.

In yet another specific embodiment, m is 21, n is 2, and X is O.

In yet another specific embodiment, m is 21, n is 2, and X is CH₂.

In yet another specific embodiment, m is 22, n is 1, and X is O.

In yet another specific embodiment, m is 22, n is 1, and X is CH₂.

In yet another specific embodiment, m is 23, n is 0, and X is O.

In yet another specific embodiment, m is 23, n is 0, and X is CH₂.

In another specific aspect, X, m and n in Formula (I-7) are as described in the First Specific Embodiment for Formula (I-5); and R and R¹ are as described above for Formula (I-7).

In another specific aspect, X, m and n in Formula (I-7) are as described in the Second Specific Embodiment for Formula (I-5) and R and R¹ are as described above for Formula (I-7).

In another specific aspect, X, m and n in Formula (I-7) are as described in the Third Specific Embodiment for Formula (I-5) and R and R¹ are as described above for Formula (I-7).

In another specific aspect, X, m and n in Formula (I-7) are as described in the Fourth Specific Embodiment for Formula (I-5) and R and R¹ are as described above for Formula (I-7).

In another specific aspect, X, m and n in Formula (I-7) are as described in the Fifth Specific Embodiment for Formula (I-5) and R and R¹ are as described above for Formula (I-7).

In another specific aspect, X, m and n in Formula (I-7) are as described in the Sixth Specific Embodiment for Formula (I-5) and R and R¹ are as described above for Formula (I-7).

Pharmaceutically acceptable salts of any one of the foregoing embodiments described in the 8^{th} aspect are also included.

In a 9^{th} aspect, provided herein is a compound, or pharmaceutically acceptable salt thereof, having a structure provided in Formula (I-8),

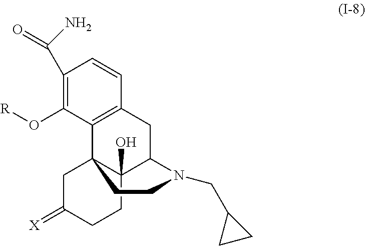

wherein:
X is O or CH₂;
R is —C(O)OR¹;
wherein R¹ is

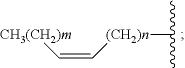

and
wherein m and n are integers between 0 and 23, and the sum of m and n is an integer between 6 and 23.

In one specific embodiment, m is 0, n is 23, and X is O.

In another specific embodiment, m is 0, n is 23, and X is CH₂.

In yet another specific embodiment, m is 1, n is 22, and X is O.

In yet another specific embodiment, m is 1, n is 22, and X is CH₂.

In yet another specific embodiment, m is 2, n is 21, and X is O.

In yet another specific embodiment, m is 2, n is 21, and X is CH₂.

In yet another specific embodiment, m is 3, n is 20, and X is O.

In yet another specific embodiment, m is 3, n is 20, and X is CH₂.

In yet another specific embodiment, m is 4, n is 19, and X is O.

In yet another specific embodiment, m is 4, n is 19, and X is CH₂.

In yet another specific embodiment, m is 5, n is 18, and X is O.

In yet another specific embodiment, m is 5, n is 18, and X is CH₂.

In yet another specific embodiment, m is 6, n is 17, and X is O.

In yet another specific embodiment, m is 6, n is 17, and X is CH₂.

In yet another specific embodiment, m is 7, n is 16, and X is O.

In yet another specific embodiment, m is 7, n is 16, and X is CH₂.

In yet another specific embodiment, m is 8, n is 15, and X is O.

In yet another specific embodiment, m is 8, n is 15, and X is CH₂.

In yet another specific embodiment, m is 9, n is 14, and X is O.

In yet another specific embodiment, m is 9, n is 14, and X is CH₂.

In yet another specific embodiment, m is 10, n is 13, and X is O.

In yet another specific embodiment, m is 10, n is 13, and X is CH₂.

In yet another specific embodiment, m is 11, n is 12, and X is O.

In yet another specific embodiment, m is 11, n is 12, and X is CH₂.

In yet another specific embodiment, m is 12, n is 11, and X is O.

In yet another specific embodiment, m is 12, n is 11, and X is CH₂.

In yet another specific embodiment, m is 13, n is 10, and X is O.

In yet another specific embodiment, m is 13, n is 10, and X is CH₂.

In yet another specific embodiment, m is 14, n is 9, and X is O.

In yet another specific embodiment, m is 14, n is 9, and X is CH₂.

In yet another specific embodiment, m is 15, n is 8, and X is O.

In yet another specific embodiment, m is 15, n is 8, and X is CH₂.

In yet another specific embodiment, m is 16, n is 7, and X is O.

In yet another specific embodiment, m is 16, n is 7, and X is CH₂.

In yet another specific embodiment, m is 17, n is 6, and X is O.

In yet another specific embodiment, m is 17, n is 6, and X is CH₂.

In yet another specific embodiment, m is 18, n is 5, and X is O.

In yet another specific embodiment, m is 18, n is 5, and X is CH₂.

In yet another specific embodiment, m is 19, n is 4, and X is O.

In yet another specific embodiment, m is 19, n is 4, and X is CH₂.

In yet another specific embodiment, m is 20, n is 3, and X is O.

In yet another specific embodiment, m is 20, n is 3, and X is CH₂.

In yet another specific embodiment, m is 21, n is 2, and X is O.

In yet another specific embodiment, m is 21, n is 2, and X is CH₂.

In yet another specific embodiment, m is 22, n is 1, and X is O.

In yet another specific embodiment, m is 22, n is 1, and X is CH₂.

In yet another specific embodiment, m is 23, n is 0, and X is O.

In yet another specific embodiment, m is 23, n is 0, and X is CH₂.

In another specific aspect, X, m and n in Formula (I-8) are as described in the First Specific Embodiment for Formula (I-5)) and R and R¹ are as described above for Formula (I-8).

In another specific aspect, X, m and n in Formula (I-8) are as described in the Second Specific Embodiment for Formula (I-5) and R and R¹ are as described above for Formula (I-8).

In another specific aspect, X, m and n in Formula (I-8) are as described in the Third Specific Embodiment for Formula (I-5) and R and R¹ are as described above for Formula (I-8).

In another specific aspect, X, m and n in Formula (I-8) are as described in the Fourth Specific Embodiment for Formula (I-5) and R and R¹ are as described above for Formula (I-8).

In another specific aspect, X, m and n in Formula (I-8) are as described in the Fifth Specific Embodiment for Formula (I-5) and R and R¹ are as described above for Formula (I-8).

In another specific aspect, X, m and n in Formula (I-8) are as described in the Sixth Specific Embodiment for Formula (I-5) and R and R¹ are as described above for Formula (I-8).

Pharmaceutically acceptable salts of any one of the foregoing embodiments described in the 9^{th} aspect are also included.

In a 10^{th} aspect, provided herein is a compound, or pharmaceutically acceptable salt thereof, having a structure provided in Formula (I-9),

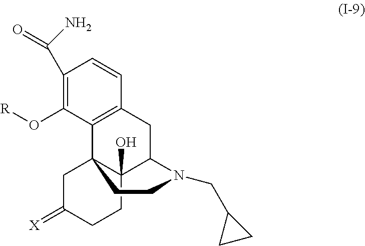

wherein:
X is O or CH₂;
R is —CH₂OC(O)R¹;
wherein R¹ is

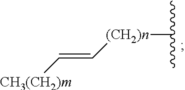

and
wherein m and n are integers between 0 and 23, and the sum of m and n is an integer between 6 and 23.

In one specific embodiment, m is 0, n is 23, and X is O.

In another specific embodiment, m is 0, n is 23, and X is CH₂.

In yet another specific embodiment, m is 1, n is 22, and X is O.

In yet another specific embodiment, m is 1, n is 22, and X is CH₂.

In yet another specific embodiment, m is 2, n is 21, and X is O.

In yet another specific embodiment, m is 2, n is 21, and X is CH₂.

In yet another specific embodiment, m is 3, n is 20, and X is O.

In yet another specific embodiment, m is 3, n is 20, and X is CH₂.

In yet another specific embodiment, m is 4, n is 19, and X is O.

In yet another specific embodiment, m is 4, n is 19, and X is CH₂.

In yet another specific embodiment, m is 5, n is 18, and X is O.

In yet another specific embodiment, m is 5, n is 18, and X is CH₂.

In yet another specific embodiment, m is 6, n is 17, and X is O.

In yet another specific embodiment, m is 6, n is 17, and X is CH₂.

In yet another specific embodiment, m is 7, n is 16, and X is O.

In yet another specific embodiment, m is 7, n is 16, and X is CH₂.

In yet another specific embodiment, m is 8, n is 15, and X is O.

In yet another specific embodiment, m is 8, n is 15, and X is CH₂.

In yet another specific embodiment, m is 9, n is 14, and X is O.

In yet another specific embodiment, m is 9, n is 14, and X is CH₂.

In yet another specific embodiment, m is 10, n is 13, and X is O.

In yet another specific embodiment, m is 10, n is 13, and X is CH₂.

In yet another specific embodiment, m is 11, n is 12, and X is O.

In yet another specific embodiment, m is 11, n is 12, and X is CH₂.

In yet another specific embodiment, m is 12, n is 11, and X is O.

In yet another specific embodiment, m is 12, n is 11, and X is CH₂.

In yet another specific embodiment, m is 13, n is 10, and X is O.

In yet another specific embodiment, m is 13, n is 10, and X is CH₂.

In yet another specific embodiment, m is 14, n is 9, and X is O.

In yet another specific embodiment, m is 14, n is 9, and X is CH₂.

In yet another specific embodiment, m is 15, n is 8, and X is O.

In yet another specific embodiment, m is 15, n is 8, and X is CH₂.

In yet another specific embodiment, m is 16, n is 7, and X is O.

In yet another specific embodiment, m is 16, n is 7, and X is CH₂.

In yet another specific embodiment, m is 17, n is 6, and X is O.

In yet another specific embodiment, m is 17, n is 6, and X is CH₂.

In yet another specific embodiment, m is 18, n is 5, and X is O.

In yet another specific embodiment, m is 18, n is 5, and X is CH₂.

In yet another specific embodiment, m is 19, n is 4, and X is O.

In yet another specific embodiment, m is 19, n is 4, and X is CH₂.

In yet another specific embodiment, m is 20, n is 3, and X is O.

In yet another specific embodiment, m is 20, n is 3, and X is CH₂.

In yet another specific embodiment, m is 21, n is 2, and X is O.

In yet another specific embodiment, m is 21, n is 2, and X is CH₂.

In yet another specific embodiment, m is 22, n is 1, and X is O.

In yet another specific embodiment, m is 22, n is 1, and X is CH₂.

In yet another specific embodiment, m is 23, n is 0, and X is O.

In yet another specific embodiment, m is 23, n is 0, and X is CH₂.

In another specific aspect, X, m and n in Formula (I-9) are as described in the First Specific Embodiment for Formula (I-5) and R and R¹ are as described above for Formula (I-9).

In another specific aspect, X, m and n in Formula (I-9) are as described in the Second Specific Embodiment for Formula (I-5) and R and R¹ are as described above for Formula (I-9).

In another specific aspect, X, m and n in Formula (I-9) are as described in the Third Specific Embodiment for Formula (I-5) and R and R¹ are as described above for Formula (I-9).

In another specific aspect, X, m and n in Formula (I-9) are as described in the Fourth Specific Embodiment for Formula (I-5) and R and R¹ are as described above for Formula (I-9).

In another specific aspect, X, m and n in Formula (I-9) are as described in the Fifth Specific Embodiment for Formula (I-5) and R and R¹ are as described above for Formula (I-9).

In another specific aspect, X, m and n in Formula (I-9) are as described in the Sixth Specific Embodiment for Formula (I-5) and R and R¹ are as described above for Formula (I-9).

Pharmaceutically acceptable salts of any one of the foregoing embodiments described in the 10^{th} aspect are also included.

In an 11^{th} aspect, provided herein is a compound, or pharmaceutically acceptable salt thereof, having a structure provided in Formula (I-10),

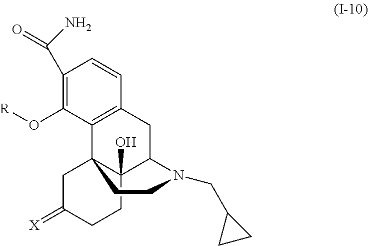

wherein:
X is O or CH₂;
R is —CH₂OC(O)R¹;
wherein R¹ is

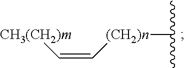

and
wherein m and n are integers between 0 and 23, and the sum of m and n is an integer between 6 and 23.

In one specific embodiment, m is 0, n is 23, and X is O.

In another specific embodiment, m is 0, n is 23, and X is CH₂.

In yet another specific embodiment, m is 1, n is 22, and X is O.

In yet another specific embodiment, m is 1, n is 22, and X is CH₂.

In yet another specific embodiment, m is 2, n is 21, and X is O.

In yet another specific embodiment, m is 2, n is 21, and X is CH₂.

In yet another specific embodiment, m is 3, n is 20, and X is O.

In yet another specific embodiment, m is 3, n is 20, and X is CH₂.

In yet another specific embodiment, m is 4, n is 19, and X is O.

In yet another specific embodiment, m is 4, n is 19, and X is CH₂.

In yet another specific embodiment, m is 5, n is 18, and X is O.

In yet another specific embodiment, m is 5, n is 18, and X is CH₂.

In yet another specific embodiment, m is 6, n is 17, and X is O.

In yet another specific embodiment, m is 6, n is 17, and X is CH₂.

In yet another specific embodiment, m is 7, n is 16, and X is O.

In yet another specific embodiment, m is 7, n is 16, and X is CH₂.

In yet another specific embodiment, m is 8, n is 15, and X is O.

In yet another specific embodiment, m is 8, n is 15, and X is CH₂.

In yet another specific embodiment, m is 9, n is 14, and X is O.

In yet another specific embodiment, m is 9, n is 14, and X is CH₂.

In yet another specific embodiment, m is 10, n is 13, and X is O.

In yet another specific embodiment, m is 10, n is 13, and X is CH₂.

In yet another specific embodiment, m is 11, n is 12, and X is O.

In yet another specific embodiment, m is 11, n is 12, and X is CH₂.

In yet another specific embodiment, m is 12, n is 11, and X is O.

In yet another specific embodiment, m is 12, n is 11, and X is CH₂.

In yet another specific embodiment, m is 13, n is 10, and X is O.

In yet another specific embodiment, m is 13, n is 10, and X is CH₂.

In yet another specific embodiment, m is 14, n is 9, and X is O.

In yet another specific embodiment, m is 14, n is 9, and X is CH₂.

In yet another specific embodiment, m is 15, n is 8, and X is O.

In yet another specific embodiment, m is 15, n is 8, and X is CH₂.

In yet another specific embodiment, m is 16, n is 7, and X is O.

In yet another specific embodiment, m is 16, n is 7, and X is CH₂.

In yet another specific embodiment, m is 17, n is 6, and X is O.

In yet another specific embodiment, m is 17, n is 6, and X is CH₂.

In yet another specific embodiment, m is 18, n is 5, and X is O.

In yet another specific embodiment, m is 18, n is 5, and X is CH₂.

In yet another specific embodiment, m is 19, n is 4, and X is O.

In yet another specific embodiment, m is 19, n is 4, and X is CH₂.

In yet another specific embodiment, m is 20, n is 3, and X is O.

In yet another specific embodiment, m is 20, n is 3, and X is CH₂.

In yet another specific embodiment, m is 21, n is 2, and X is O.

In yet another specific embodiment, m is 21, n is 2, and X is CH₂.

In yet another specific embodiment, m is 22, n is 1, and X is O.

In yet another specific embodiment, m is 22, n is 1, and X is CH₂.

In yet another specific embodiment, m is 23, n is 0, and X is O.

In yet another specific embodiment, m is 23, n is 0, and X is CH₂.

In another specific aspect, X, m and n in Formula (I-10) are as described in the First Specific Embodiment for Formula (I-5) and R and R¹ are as described above for Formula (I-10).

In another specific aspect, X, m and n in Formula (I-10) are as described in the Second Specific Embodiment for Formula (I-5) and R and R¹ are as described above for Formula (I-10).

In another specific aspect, X, m and n in Formula (I-10) are as described in the Third Specific Embodiment for Formula (I-5) and R and R¹ are as described above for Formula (I-10).

In another specific aspect, X, m and n in Formula (I-10) are as described in the Fourth Specific Embodiment for Formula (I-5) and R and R¹ are as described above for Formula (I-10).

In another specific aspect, X, m and n in Formula (I-10) are as described in the Fifth Specific Embodiment for Formula (I-5) and R and R¹ are as described above for Formula (I-10).

In another specific aspect, X, m and n in Formula (I-10) are as described in the Sixth Specific Embodiment for Formula (I-5) and R and R¹ are as described above for Formula (I-10).

Pharmaceutically acceptable salts of any one of the foregoing embodiments described in the 11^{th} aspect are also included.

In a 12^{th} aspect, provided herein is a compound, or pharmaceutically acceptable salt thereof, having a structure provided in Formula (I-11),

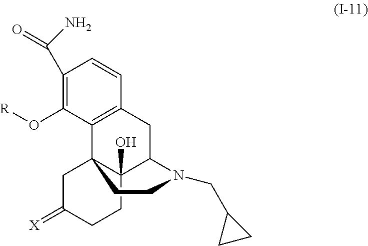

wherein:
X is O or CH₂;
R is —CH₂OC(O)OR¹;
wherein R¹ is

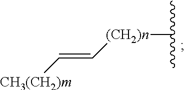

and
wherein m and n are integers between 0 and 23, and the sum of m and n is an integer between 6 and 23.

In one specific embodiment, m is 0, n is 23, and X is O.

In another specific embodiment, m is 0, n is 23, and X is CH₂.

In yet another specific embodiment, m is 1, n is 22, and X is O.

In yet another specific embodiment, m is 1, n is 22, and X is CH₂.

In yet another specific embodiment, m is 2, n is 21, and X is O.

In yet another specific embodiment, m is 2, n is 21, and X is CH₂.

In yet another specific embodiment, m is 3, n is 20, and X is O.

In yet another specific embodiment, m is 3, n is 20, and X is CH₂.

In yet another specific embodiment, m is 4, n is 19, and X is O.

In yet another specific embodiment, m is 4, n is 19, and X is CH₂.

In yet another specific embodiment, m is 5, n is 18, and X is O.

In yet another specific embodiment, m is 5, n is 18, and X is CH₂.

In yet another specific embodiment, m is 6, n is 17, and X is O.

In yet another specific embodiment, m is 6, n is 17, and X is CH₂.

In yet another specific embodiment, m is 7, n is 16, and X is O.

In yet another specific embodiment, m is 7, n is 16, and X is CH₂.

In yet another specific embodiment, m is 8, n is 15, and X is O.

In yet another specific embodiment, m is 8, n is 15, and X is CH₂.

In yet another specific embodiment, m is 9, n is 14, and X is O.

In yet another specific embodiment, m is 9, n is 14, and X is CH₂.

In yet another specific embodiment, m is 10, n is 13, and X is O.

In yet another specific embodiment, m is 10, n is 13, and X is CH₂.

In yet another specific embodiment, m is 11, n is 12, and X is O.

In yet another specific embodiment, m is 11, n is 12, and X is CH₂.

In yet another specific embodiment, m is 12, n is 11, and X is O.

In yet another specific embodiment, m is 12, n is 11, and X is CH₂.

In yet another specific embodiment, m is 13, n is 10, and X is O.

In yet another specific embodiment, m is 13, n is 10, and X is CH₂.

In yet another specific embodiment, m is 14, n is 9, and X is O.

In yet another specific embodiment, m is 14, n is 9, and X is CH₂.

In yet another specific embodiment, m is 15, n is 8, and X is O.

In yet another specific embodiment, m is 15, n is 8, and X is CH₂.

In yet another specific embodiment, m is 16, n is 7, and X is O.

In yet another specific embodiment, m is 16, n is 7, and X is CH₂.

In yet another specific embodiment, m is 17, n is 6, and X is O.

In yet another specific embodiment, m is 17, n is 6, and X is CH₂.

In yet another specific embodiment, m is 18, n is 5, and X is O.

In yet another specific embodiment, m is 18, n is 5, and X is CH₂.

In yet another specific embodiment, m is 19, n is 4, and X is O.

In yet another specific embodiment, m is 19, n is 4, and X is CH₂.

In yet another specific embodiment, m is 20, n is 3, and X is O.

In yet another specific embodiment, m is 20, n is 3, and X is CH₂.

In yet another specific embodiment, m is 21, n is 2, and X is O.

In yet another specific embodiment, m is 21, n is 2, and X is CH₂.

In yet another specific embodiment, m is 22, n is 1, and X is O.

In yet another specific embodiment, m is 22, n is 1, and X is CH₂.

In yet another specific embodiment, m is 23, n is 0, and X is O.

In yet another specific embodiment, m is 23, n is 0, and X is CH₂.

In another specific aspect, X, m and n in Formula (I-11) are as described in the First Specific Embodiment for Formula (I-5) and R and R¹ are as described above for Formula (I-11).

In another specific aspect, X, m and n in Formula (I-11) are as described in the Second Specific Embodiment for Formula (I-5) and R and R¹ are as described above for Formula (I-11).

In another specific aspect, X, m and n in Formula (I-11) are as described in the Third Specific Embodiment for Formula (I-5) and R and R¹ are as described above for Formula (I-11).

In another specific aspect, X, m and n in Formula (I-11) are as described in the Fourth Specific Embodiment for Formula (I-5) and R and R¹ are as described above for Formula (I-11).

In another specific aspect, X, m and n in Formula (I-11) are as described in the Fifth Specific Embodiment for Formula (I-5) and R and R¹ are as described above for Formula (I-11).

In another specific aspect, X, m and n in Formula (I-11) are as described in the Sixth Specific Embodiment for Formula (I-5) and R and R¹ are as described above for Formula (I-11).

Pharmaceutically acceptable salts of any one of the foregoing embodiments described in the 12^{th} aspect are also included.

In a 13^{th} aspect, provided herein is a compound, or pharmaceutically acceptable salt thereof, having a structure provided in Formula (I-12),

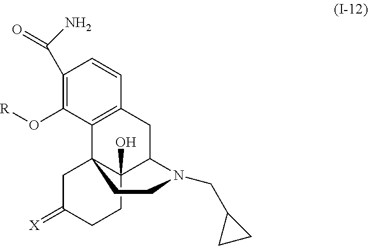

wherein:
X is O or CH₂;
R is —CH₂OC(O)OR¹;
wherein R¹ is

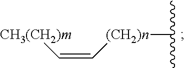

and
wherein m and n are integers between 0 and 23, and the sum of m and n is an integer between 6 and 23.

In one specific embodiment, m is 0, n is 23, and X is O.

In another specific embodiment, m is 0, n is 23, and X is CH₂.

In yet another specific embodiment, m is 1, n is 22, and X is O.

In yet another specific embodiment, m is 1, n is 22, and X is CH₂.

In yet another specific embodiment, m is 2, n is 21, and X is O.

In yet another specific embodiment, m is 2, n is 21, and X is CH₂.

In yet another specific embodiment, m is 3, n is 20, and X is O.

In yet another specific embodiment, m is 3, n is 20, and X is CH₂.

In yet another specific embodiment, m is 4, n is 19, and X is O.

In yet another specific embodiment, m is 4, n is 19, and X is CH₂.

In yet another specific embodiment, m is 5, n is 18, and X is O.

In yet another specific embodiment, m is 5, n is 18, and X is CH₂.

In yet another specific embodiment, m is 6, n is 17, and X is O.

In yet another specific embodiment, m is 6, n is 17, and X is CH₂.

In yet another specific embodiment, m is 7, n is 16, and X is O.

In yet another specific embodiment, m is 7, n is 16, and X is CH₂.

In yet another specific embodiment, m is 8, n is 15, and X is O.

In yet another specific embodiment, m is 8, n is 15, and X is CH₂.

In yet another specific embodiment, m is 9, n is 14, and X is O.

In yet another specific embodiment, m is 9, n is 14, and X is CH₂.

In yet another specific embodiment, m is 10, n is 13, and X is O.

In yet another specific embodiment, m is 10, n is 13, and X is CH₂.

In yet another specific embodiment, m is 11, n is 12, and X is O.

In yet another specific embodiment, m is 11, n is 12, and X is CH₂.

In yet another specific embodiment, m is 12, n is 11, and X is O.

In yet another specific embodiment, m is 12, n is 11, and X is CH₂.

In yet another specific embodiment, m is 13, n is 10, and X is O.

In yet another specific embodiment, m is 13, n is 10, and X is CH₂.

In yet another specific embodiment, m is 14, n is 9, and X is O.

In yet another specific embodiment, m is 14, n is 9, and X is CH₂.

In yet another specific embodiment, m is 15, n is 8, and X is O.

In yet another specific embodiment, m is 15, n is 8, and X is CH₂.

In yet another specific embodiment, m is 16, n is 7, and X is O.

In yet another specific embodiment, m is 16, n is 7, and X is CH₂.

In yet another specific embodiment, m is 17, n is 6, and X is O.

In yet another specific embodiment, m is 17, n is 6, and X is CH₂.

In yet another specific embodiment, m is 18, n is 5, and X is O.

In yet another specific embodiment, m is 18, n is 5, and X is CH₂.

In yet another specific embodiment, m is 19, n is 4, and X is O.

In yet another specific embodiment, m is 19, n is 4, and X is CH₂.

In yet another specific embodiment, m is 20, n is 3, and X is O.

In yet another specific embodiment, m is 20, n is 3, and X is CH₂.

In yet another specific embodiment, m is 21, n is 2, and X is O.

In yet another specific embodiment, m is 21, n is 2, and X is CH₂.

In yet another specific embodiment, m is 22, n is 1, and X is O.

In yet another specific embodiment, m is 22, n is 1, and X is CH₂.

In yet another specific embodiment, m is 23, n is 0, and X is O.

In yet another specific embodiment, m is 23, n is 0, and X is CH₂.

In another specific aspect, X, m and n in Formula (I-12) are as described in the First Specific Embodiment for Formula (I-5) and R and R¹ are as described above for Formula (I-12).

In another specific aspect, X, m and n in Formula (I-12) are as described in the Second Specific Embodiment for Formula (I-5) and R and R¹ are as described above for Formula (I-12).

In another specific aspect, X, m and n in Formula (I-12) are as described in the Third Specific Embodiment for Formula (I-5) and R and R¹ are as described above for Formula (I-12).

In another specific aspect, X, m and n in Formula (I-12) are as described in the Fourth Specific Embodiment for Formula (I-5) and R and R¹ are as described above for Formula (I-12).

In another specific aspect, X, m and n in Formula (I-12) are as described in the Fifth Specific Embodiment for Formula (I-5) and R and R¹ are as described above for Formula (I-12).

In another specific aspect, X, m and n in Formula (I-12) are as described in the Sixth Specific Embodiment for Formula (I-5) and R and R¹ are as described above for Formula (I-12).

Pharmaceutically acceptable salts of any one of the foregoing embodiments described in the 13^{th} aspect are also included.

In a 14^{th} aspect, provided herein is a compound, or pharmaceutically acceptable salt thereof, having a structure provided in Formula (II):

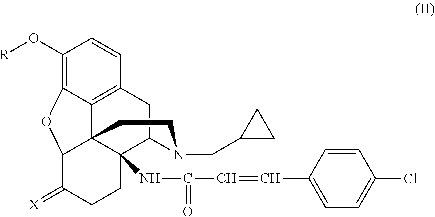

wherein:
X is O or CH₂;
R is selected from the group consisting of:
   (a) —C(O)R¹,
   (b) —C(O)OR¹,
   (c) —CH₂OC(O)R¹,
   (d) —CH₂OC(O)OR¹,
   (e) —CH₂OR¹;
each instance of R¹ is independently —(CH₂)_{q}CH₃,

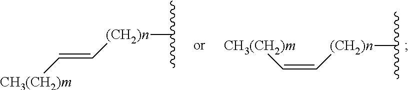

m and n are integers between 0 and 23, and the sum of m and n is an integer between 6 and 23; and
q is an integer between 0 and 25.

In a 15^{th} aspect, provided herein is a compound, or pharmaceutically acceptable salt thereof, having a structure provided in Formula (II-1),

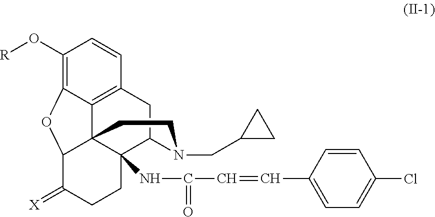

wherein:
X is O or CH₂;
R is —C(O)R¹;
wherein R¹ is —(CH₂)_{q}CH₃; and
wherein q is an integer between 0 and 25.

In one specific embodiment, q is 0 and X is O.

In another specific embodiment, q is 0 and X is CH₂.

In yet another specific embodiment, q is 1 and X is O.

In yet another specific embodiment, q is 1 and X is CH₂.

In yet another specific embodiment, q is 2 and X is O.

In yet another specific embodiment, q is 2 and X is CH₂.

In yet another specific embodiment, q is 3 and X is O.

In yet another specific embodiment, q is 3 and X is CH₂.

In yet another specific embodiment, q is 4 and X is O.

In yet another specific embodiment, q is 4 and X is CH₂.

In yet another specific embodiment, q is 5 and X is O.

In yet another specific embodiment, q is 5 and X is CH₂.

In yet another specific embodiment, q is 6 and X is O.

In yet another specific embodiment, q is 6 and X is CH₂.

In yet another specific embodiment, q is 7 and X is O.

In yet another specific embodiment, q is 7 and X is CH₂.

In yet another specific embodiment, q is 8 and X is O.

In yet another specific embodiment, q is 8 and X is CH₂.

In yet another specific embodiment, q is 9 and X is O.

In yet another specific embodiment, q is 9 and X is CH₂.

In yet another specific embodiment, q is 10 and X is O.

In yet another specific embodiment, q is 10 and X is CH₂.

In yet another specific embodiment, q is 11 and X is O.

In yet another specific embodiment, q is 11 and X is CH₂.

In yet another specific embodiment, q is 12 and X is O.

In yet another specific embodiment, q is 12 and X is CH₂.

In yet another specific embodiment, q is 13 and X is O.

In yet another specific embodiment, q is 13 and X is CH₂.

In yet another specific embodiment, q is 14 and X is O.

In yet another specific embodiment, q is 14 and X is CH₂.

In yet another specific embodiment, q is 15 and X is O.

In yet another specific embodiment, q is 15 and X is CH₂.

In yet another specific embodiment, q is 16 and X is O.

In yet another specific embodiment, q is 16 and X is CH₂.

In yet another specific embodiment, q is 17 and X is O.

In yet another specific embodiment, q is 17 and X is CH₂.

In yet another specific embodiment, q is 18 and X is O.

In yet another specific embodiment, q is 18 and X is CH₂.

In yet another specific embodiment, q is 19 and X is O.

In yet another specific embodiment, q is 19 and X is CH₂.

In yet another specific embodiment, q is 20 and X is O.

In yet another specific embodiment, q is 20 and X is CH₂.

In yet another specific embodiment, q is 21 and X is O.

In yet another specific embodiment, q is 21 and X is CH₂.

In yet another specific embodiment, q is 22 and X is O.

In yet another specific embodiment, q is 22 and X is CH₂.

In yet another specific embodiment, q is 23 and X is O.

In yet another specific embodiment, q is 23 and X is CH₂.

In yet another specific embodiment, q is 24 and X is O.

In yet another specific embodiment, q is 24 and X is CH₂.

In yet another specific embodiment, q is 25 and X is O.

In yet another specific embodiment, q is 25 and X is CH₂.

Pharmaceutically acceptable salts of any one of the foregoing embodiments described in the 15^{th} aspect are also included.

In a 16^{th} aspect, provided herein is a compound, or pharmaceutically acceptable salt thereof, having a structure provided in Formula (II-2),

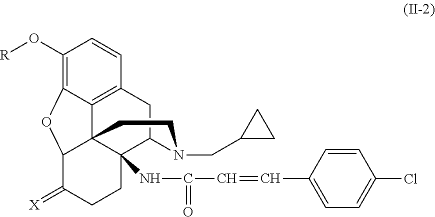

wherein:
X is O or CH₂;
R is —C(O)OR¹;
wherein R¹ is —(CH₂)_{q}CH₃; and
wherein q is an integer between 0 and 25.

In one specific embodiment, q is 0 and X is O.

In another specific embodiment, q is 0 and X is CH₂.

In yet another specific embodiment, q is 1 and X is O.

In yet another specific embodiment, q is 1 and X is CH₂.

In yet another specific embodiment, q is 2 and X is O.

In yet another specific embodiment, q is 2 and X is CH₂.

In yet another specific embodiment, q is 3 and X is O.

In yet another specific embodiment, q is 3 and X is CH₂.

In yet another specific embodiment, q is 4 and X is O.

In yet another specific embodiment, q is 4 and X is CH₂.

In yet another specific embodiment, q is 5 and X is O.

In yet another specific embodiment, q is 5 and X is CH₂.

In yet another specific embodiment, q is 6 and X is O.

In yet another specific embodiment, q is 6 and X is CH₂.

In yet another specific embodiment, q is 7 and X is O.

In yet another specific embodiment, q is 7 and X is CH₂.

In yet another specific embodiment, q is 8 and X is O.

In yet another specific embodiment, q is 8 and X is CH₂.

In yet another specific embodiment, q is 9 and X is O.

In yet another specific embodiment, q is 9 and X is CH₂.

In yet another specific embodiment, q is 10 and X is O.

In yet another specific embodiment, q is 10 and X is CH₂.

In yet another specific embodiment, q is 11 and X is O.

In yet another specific embodiment, q is 11 and X is CH₂.

In yet another specific embodiment, q is 12 and X is O.

In yet another specific embodiment, q is 12 and X is CH₂.

In yet another specific embodiment, q is 13 and X is O.

In yet another specific embodiment, q is 13 and X is CH₂.

In yet another specific embodiment, q is 14 and X is O.

In yet another specific embodiment, q is 14 and X is CH₂.

In yet another specific embodiment, q is 15 and X is O.

In yet another specific embodiment, q is 15 and X is CH₂.

In yet another specific embodiment, q is 16 and X is O.

In yet another specific embodiment, q is 16 and X is CH₂.

In yet another specific embodiment, q is 17 and X is O.

In yet another specific embodiment, q is 17 and X is CH₂.

In yet another specific embodiment, q is 18 and X is O.

In yet another specific embodiment, q is 18 and X is CH₂.

In yet another specific embodiment, q is 19 and X is O.

In yet another specific embodiment, q is 19 and X is CH₂.

In yet another specific embodiment, q is 20 and X is O.

In yet another specific embodiment, q is 20 and X is CH₂.

In yet another specific embodiment, q is 21 and X is O.

In yet another specific embodiment, q is 21 and X is CH₂.

In yet another specific embodiment, q is 22 and X is O.

In yet another specific embodiment, q is 22 and X is CH₂.

In yet another specific embodiment, q is 23 and X is O.

In yet another specific embodiment, q is 23 and X is CH₂.

In yet another specific embodiment, q is 24 and X is O.

In yet another specific embodiment, q is 24 and X is CH₂.

In yet another specific embodiment, q is 25 and X is O.

In yet another specific embodiment, q is 25 and X is CH₂.

Pharmaceutically acceptable salts of any one of the foregoing embodiments described in the 16^{th} aspect are also included.

In a 17^{th} aspect, provided herein is a compound, or pharmaceutically acceptable salt thereof, having a structure provided in Formula (II-3),

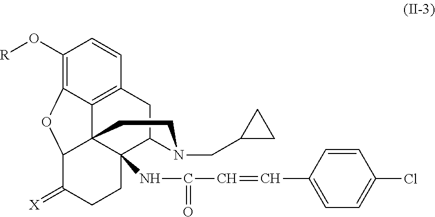

wherein:
X is O or CH₂;
R is —CH₂OC(O)R¹;
wherein R¹ is —(CH₂)_{q}CH₃; and
wherein q is an integer between 0 and 25.

In one specific embodiment, q is 0 and X is O.

In another specific embodiment, q is 0 and X is CH₂.

In yet another specific embodiment, q is 1 and X is O.

In yet another specific embodiment, q is 1 and X is CH₂.

In yet another specific embodiment, q is 2 and X is O.

In yet another specific embodiment, q is 2 and X is CH₂.

In yet another specific embodiment, q is 3 and X is O.

In yet another specific embodiment, q is 3 and X is CH₂.

In yet another specific embodiment, q is 4 and X is O.

In yet another specific embodiment, q is 4 and X is CH₂.

In yet another specific embodiment, q is 5 and X is O.

In yet another specific embodiment, q is 5 and X is CH₂.

In yet another specific embodiment, q is 6 and X is O.

In yet another specific embodiment, q is 6 and X is CH₂.

In yet another specific embodiment, q is 7 and X is O.

In yet another specific embodiment, q is 7 and X is CH₂.

In yet another specific embodiment, q is 8 and X is O.

In yet another specific embodiment, q is 8 and X is CH₂.

In yet another specific embodiment, q is 9 and X is O.

In yet another specific embodiment, q is 9 and X is CH₂.

In yet another specific embodiment, q is 10 and X is O.

In yet another specific embodiment, q is 10 and X is CH₂.

In yet another specific embodiment, q is 11 and X is O.

In yet another specific embodiment, q is 11 and X is CH₂.

In yet another specific embodiment, q is 12 and X is O.

In yet another specific embodiment, q is 12 and X is CH₂.

In yet another specific embodiment, q is 13 and X is O.

In yet another specific embodiment, q is 13 and X is CH₂.

In yet another specific embodiment, q is 14 and X is O.

In yet another specific embodiment, q is 14 and X is CH₂.

In yet another specific embodiment, q is 15 and X is O.

In yet another specific embodiment, q is 15 and X is CH₂.

In yet another specific embodiment, q is 16 and X is O.

In yet another specific embodiment, q is 16 and X is CH₂.

In yet another specific embodiment, q is 17 and X is O.

In yet another specific embodiment, q is 17 and X is CH₂.

In yet another specific embodiment, q is 18 and X is O.

In yet another specific embodiment, q is 18 and X is CH₂.

In yet another specific embodiment, q is 19 and X is O.

In yet another specific embodiment, q is 19 and X is CH₂.

In yet another specific embodiment, q is 20 and X is O.

In yet another specific embodiment, q is 20 and X is CH₂.

In yet another specific embodiment, q is 21 and X is O.

In yet another specific embodiment, q is 21 and X is CH₂.

In yet another specific embodiment, q is 22 and X is O.

In yet another specific embodiment, q is 22 and X is CH₂.

In yet another specific embodiment, q is 23 and X is O.

In yet another specific embodiment, q is 23 and X is CH₂.

In yet another specific embodiment, q is 24 and X is O.

In yet another specific embodiment, q is 24 and X is CH₂.

In yet another specific embodiment, q is 25 and X is O.

In yet another specific embodiment, q is 25 and X is CH₂.

Pharmaceutically acceptable salts of any one of the foregoing embodiments described in the 17^{th} aspect are also included.

In an 18^{th} aspect, provided herein is a compound, or pharmaceutically acceptable salt thereof, having a structure provided in Formula (II-4),

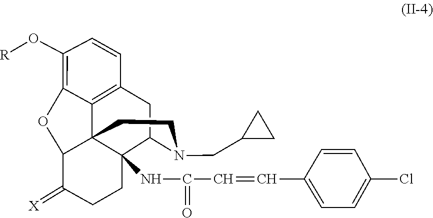

wherein:
X is O or CH₂;
R is —CH₂OC(O)OR¹;
wherein R¹ is —(CH₂)_{q}CH₃; and
wherein q is an integer between 0 and 25.

In one specific embodiment, q is 0 and X is O.

In another specific embodiment, q is 0 and X is CH₂.

In yet another specific embodiment, q is 1 and X is O.

In yet another specific embodiment, q is 1 and X is CH₂.

In yet another specific embodiment, q is 2 and X is O.

In yet another specific embodiment, q is 2 and X is CH₂.

In yet another specific embodiment, q is 3 and X is O.

In yet another specific embodiment, q is 3 and X is CH₂.

In yet another specific embodiment, q is 4 and X is O.

In yet another specific embodiment, q is 4 and X is CH₂.

In yet another specific embodiment, q is 5 and X is O.

In yet another specific embodiment, q is 5 and X is CH₂.

In yet another specific embodiment, q is 6 and X is O.

In yet another specific embodiment, q is 6 and X is CH₂.

In yet another specific embodiment, q is 7 and X is O.

In yet another specific embodiment, q is 7 and X is CH₂.

In yet another specific embodiment, q is 8 and X is O.

In yet another specific embodiment, q is 8 and X is CH₂.

In yet another specific embodiment, q is 9 and X is O.

In yet another specific embodiment, q is 9 and X is CH₂.

In yet another specific embodiment, q is 10 and X is O.

In yet another specific embodiment, q is 10 and X is CH₂.

In yet another specific embodiment, q is 11 and X is O.

In yet another specific embodiment, q is 11 and X is CH₂.

In yet another specific embodiment, q is 12 and X is O.

In yet another specific embodiment, q is 12 and X is CH₂.

In yet another specific embodiment, q is 13 and X is O.

In yet another specific embodiment, q is 13 and X is CH₂.

In yet another specific embodiment, q is 14 and X is O.

In yet another specific embodiment, q is 14 and X is CH₂.

In yet another specific embodiment, q is 15 and X is O.

In yet another specific embodiment, q is 15 and X is CH₂.

In yet another specific embodiment, q is 16 and X is O.

In yet another specific embodiment, q is 16 and X is CH₂.

In yet another specific embodiment, q is 17 and X is O.

In yet another specific embodiment, q is 17 and X is CH₂.

In yet another specific embodiment, q is 18 and X is O.

In yet another specific embodiment, q is 18 and X is CH₂.

In yet another specific embodiment, q is 19 and X is O.

In yet another specific embodiment, q is 19 and X is CH₂.

In yet another specific embodiment, q is 20 and X is O.

In yet another specific embodiment, q is 20 and X is CH₂.

In yet another specific embodiment, q is 21 and X is O.

In yet another specific embodiment, q is 21 and X is CH₂.

In yet another specific embodiment, q is 22 and X is O.

In yet another specific embodiment, q is 22 and X is CH₂.

In yet another specific embodiment, q is 23 and X is O.

In yet another specific embodiment, q is 23 and X is CH₂.

In yet another specific embodiment, q is 24 and X is O.

In yet another specific embodiment, q is 24 and X is CH₂.

In yet another specific embodiment, q is 25 and X is O.

In yet another specific embodiment, q is 25 and X is CH₂.

Pharmaceutically acceptable salts of any one of the foregoing embodiments described in the 18^{th} aspect are also included.

In a 19^{th} aspect, provided herein is a compound, or pharmaceutically acceptable salt thereof, having a structure provided in Formula (II-5),

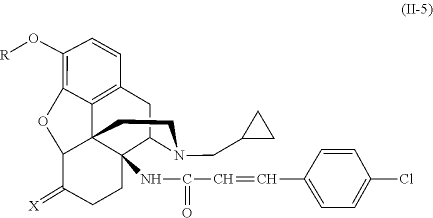

wherein:
X is O or CH₂;
R is —C(O)R¹;
wherein R¹ is

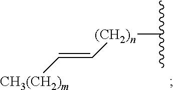

and
wherein m and n are integers between 0 and 23, and the sum of m and n is an integer between 6 and 23.

In one specific embodiment, m is 0, n is 23, and X is O.

In another specific embodiment, m is 0, n is 23, and X is CH₂.

In yet another specific embodiment, m is 1, n is 22, and X is O.

In yet another specific embodiment, m is 1, n is 22, and X is CH₂.

In yet another specific embodiment, m is 2, n is 21, and X is O.

In yet another specific embodiment, m is 2, n is 21, and X is CH₂.

In yet another specific embodiment, m is 3, n is 20, and X is O.

In yet another specific embodiment, m is 3, n is 20, and X is CH₂.

In yet another specific embodiment, m is 4, n is 19, and X is O.

In yet another specific embodiment, m is 4, n is 19, and X is CH₂.

In yet another specific embodiment, m is 5, n is 18, and X is O.

In yet another specific embodiment, m is 5, n is 18, and X is CH₂.

In yet another specific embodiment, m is 6, n is 17, and X is O.

In yet another specific embodiment, m is 6, n is 17, and X is CH₂.

In yet another specific embodiment, m is 7, n is 16, and X is O.

In yet another specific embodiment, m is 7, n is 16, and X is CH₂.

In yet another specific embodiment, m is 8, n is 15, and X is O.

In yet another specific embodiment, m is 8, n is 15, and X is CH₂.

In yet another specific embodiment, m is 9, n is 14, and X is O.

In yet another specific embodiment, m is 9, n is 14, and X is CH₂.

In yet another specific embodiment, m is 10, n is 13, and X is O.

In yet another specific embodiment, m is 10, n is 13, and X is CH₂.

In yet another specific embodiment, m is 11, n is 12, and X is O.

In yet another specific embodiment, m is 11, n is 12, and X is CH₂.

In yet another specific embodiment, m is 12, n is 11, and X is O.

In yet another specific embodiment, m is 12, n is 11, and X is CH₂.

In yet another specific embodiment, m is 13, n is 10, and X is O.

In yet another specific embodiment, m is 13, n is 10, and X is CH₂.

In yet another specific embodiment, m is 14, n is 9, and X is O.

In yet another specific embodiment, m is 14, n is 9, and X is CH₂.

In yet another specific embodiment, m is 15, n is 8, and X is O.

In yet another specific embodiment, m is 15, n is 8, and X is CH₂.

In yet another specific embodiment, m is 16, n is 7, and X is O.

In yet another specific embodiment, m is 16, n is 7, and X is CH₂.

In yet another specific embodiment, m is 17, n is 6, and X is O.

In yet another specific embodiment, m is 17, n is 6, and X is CH₂.

In yet another specific embodiment, m is 18, n is 5, and X is O.

In yet another specific embodiment, m is 18, n is 5, and X is CH₂.

In yet another specific embodiment, m is 19, n is 4, and X is O.

In yet another specific embodiment, m is 19, n is 4, and X is CH₂.

In yet another specific embodiment, m is 20, n is 3, and X is O.

In yet another specific embodiment, m is 20, n is 3, and X is CH₂.

In yet another specific embodiment, m is 21, n is 2, and X is O.

In yet another specific embodiment, m is 21, n is 2, and X is CH₂.

In yet another specific embodiment, m is 22, n is 1, and X is O.

In yet another specific embodiment, m is 22, n is 1, and X is CH₂.

In yet another specific embodiment, m is 23, n is 0, and X is O.

In yet another specific embodiment, m is 23, n is 0, and X is CH₂.

In another specific aspect, X, m and n in Formula (II-5) are as described in the First Specific Embodiment for Formula (I-5) and R and R¹ are as described above for Formula (II-5).

In another specific aspect, X, m and n in Formula (II-5) are as described in the Second Specific Embodiment for Formula (I-5) and R and R¹ are as described above for Formula (II-5).

In another specific aspect, X, m and n in Formula (II-5) are as described in the Third Specific Embodiment for Formula (I-5) and R and R¹ are as described above for Formula (II-5).

In another specific aspect, X, m and n in Formula (II-5) are as described in the Fourth Specific Embodiment for Formula (I-5) and R and R¹ are as described above for Formula (II-5).

In another specific aspect, X, m and n in Formula (II-5) are as described in the Fifth Specific Embodiment for Formula (I-5) and R and R¹ are as described above for Formula (II-5).

In another specific aspect, X, m and n in Formula (II-5) are as described in the Sixth Specific Embodiment for Formula (I-5) and R and R¹ are as described above for Formula (II-5).

Pharmaceutically acceptable salts of any one of the foregoing embodiments described in the 19^{th} aspect are also included.

In a 20^{th} aspect, provided herein is a compound, or pharmaceutically acceptable salt thereof, having a structure provided in Formula (II-6),

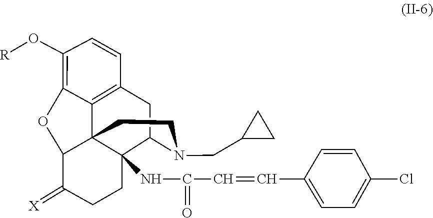

wherein:
X is O or CH₂;
R is —C(O)R¹;
wherein R¹ is

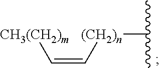

and
wherein m and n are integers between 0 and 23, and the sum of m and n is an integer between 6 and 23.

In one specific embodiment, m is 0, n is 23, and X is O.

In another specific embodiment, m is 0, n is 23, and X is CH₂.

In yet another specific embodiment, m is 1, n is 22, and X is O.

In yet another specific embodiment, m is 1, n is 22, and X is CH₂.

In yet another specific embodiment, m is 2, n is 21, and X is O.

In yet another specific embodiment, m is 2, n is 21, and X is CH₂.

In yet another specific embodiment, m is 3, n is 20, and X is O.

In yet another specific embodiment, m is 3, n is 20, and X is CH₂.

In yet another specific embodiment, m is 4, n is 19, and X is O.

In yet another specific embodiment, m is 4, n is 19, and X is CH₂.

In yet another specific embodiment, m is 5, n is 18, and X is O.

In yet another specific embodiment, m is 5, n is 18, and X is CH₂.

In yet another specific embodiment, m is 6, n is 17, and X is O.

In yet another specific embodiment, m is 6, n is 17, and X is CH₂.

In yet another specific embodiment, m is 7, n is 16, and X is O.

In yet another specific embodiment, m is 7, n is 16, and X is CH₂.

In yet another specific embodiment, m is 8, n is 15, and X is O.

In yet another specific embodiment, m is 8, n is 15, and X is CH₂.

In yet another specific embodiment, m is 9, n is 14, and X is O.

In yet another specific embodiment, m is 9, n is 14, and X is CH₂.

In yet another specific embodiment, m is 10, n is 13, and X is O.

In yet another specific embodiment, m is 10, n is 13, and X is CH₂.

In yet another specific embodiment, m is 11, n is 12, and X is O.

In yet another specific embodiment, m is 11, n is 12, and X is CH₂.

In yet another specific embodiment, m is 12, n is 11, and X is O.

In yet another specific embodiment, m is 12, n is 11, and X is CH₂.

In yet another specific embodiment, m is 13, n is 10, and X is O.

In yet another specific embodiment, m is 13, n is 10, and X is CH₂.

In yet another specific embodiment, m is 14, n is 9, and X is O.

In yet another specific embodiment, m is 14, n is 9, and X is CH₂.

In yet another specific embodiment, m is 15, n is 8, and X is O.

In yet another specific embodiment, m is 15, n is 8, and X is CH₂.

In yet another specific embodiment, m is 16, n is 7, and X is O.

In yet another specific embodiment, m is 16, n is 7, and X is CH₂.

In yet another specific embodiment, m is 17, n is 6, and X is O.

In yet another specific embodiment, m is 17, n is 6, and X is CH₂.

In yet another specific embodiment, m is 18, n is 5, and X is O.

In yet another specific embodiment, m is 18, n is 5, and X is CH₂.

In yet another specific embodiment, m is 19, n is 4, and X is O.

In yet another specific embodiment, m is 19, n is 4, and X is CH₂.

In yet another specific embodiment, m is 20, n is 3, and X is O.

In yet another specific embodiment, m is 20, n is 3, and X is CH₂.

In yet another specific embodiment, m is 21, n is 2, and X is O.

In yet another specific embodiment, m is 21, n is 2, and X is CH₂.

In yet another specific embodiment, m is 22, n is 1, and X is O.

In yet another specific embodiment, m is 22, n is 1, and X is CH₂.

In yet another specific embodiment, m is 23, n is 0, and X is O.

In yet another specific embodiment, m is 23, n is 0, and X is CH₂.

In another specific aspect, X, m and n in Formula (II-6) are as described in the First Specific Embodiment for Formula (I-5); and R and R¹ are as described above for Formula (II-6).

In another specific aspect, X, m and n in Formula (II-6) are as described in the Second Specific Embodiment for Formula (I-5); and R and R¹ are as described above for Formula (II-6).

In another specific aspect, X, m and n in Formula (II-6) are as described in the Third Specific Embodiment for Formula (I-5); and R and R¹ are as described above for Formula (II-6).

In another specific aspect, X, m and n in Formula (II-6) are as described in the Fourth Specific Embodiment for Formula (I-5); and R and R¹ are as described above for Formula (II-6).

In another specific aspect, X, m and n in Formula (II-6) are as described in the Fifth Specific Embodiment for Formula (I-5); and R and R¹ are as described above for Formula (II-6).

In another specific aspect, X, m and n in Formula (II-6) are as described in the Sixth Specific Embodiment for Formula (I-5); and R and R¹ are as described above for Formula (II-6).

Pharmaceutically acceptable salts of any one of the foregoing embodiments described in the 20^{th} aspect are also included.

In a 21^{st} aspect, provided herein is a compound, or pharmaceutically acceptable salt thereof, having a structure provided in Formula (II-7),

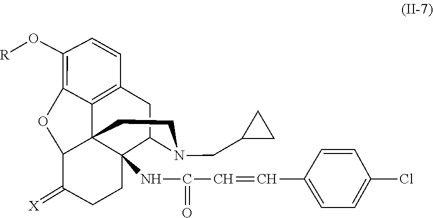

wherein:
X is O or CH₂;
R is —C(O)OR¹;
wherein R¹ is

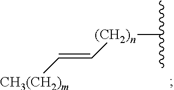

and
wherein m and n are integers between 0 and 23, and the sum of m and n is an integer between 6 and 23.

In one specific embodiment, m is 0, n is 23, and X is O.

In another specific embodiment, m is 0, n is 23, and X is CH₂.

In yet another specific embodiment, m is 1, n is 22, and X is O.

In yet another specific embodiment, m is 1, n is 22, and X is CH₂.

In yet another specific embodiment, m is 2, n is 21, and X is O.

In yet another specific embodiment, m is 2, n is 21, and X is CH₂.

In yet another specific embodiment, m is 3, n is 20, and X is O.

In yet another specific embodiment, m is 3, n is 20, and X is CH₂.

In yet another specific embodiment, m is 4, n is 19, and X is O.

In yet another specific embodiment, m is 4, n is 19, and X is CH₂.

In yet another specific embodiment, m is 5, n is 18, and X is O.

In yet another specific embodiment, m is 5, n is 18, and X is CH₂.

In yet another specific embodiment, m is 6, n is 17, and X is O.

In yet another specific embodiment, m is 6, n is 17, and X is CH₂.

In yet another specific embodiment, m is 7, n is 16, and X is O.

In yet another specific embodiment, m is 7, n is 16, and X is CH₂.

In yet another specific embodiment, m is 8, n is 15, and X is O.

In yet another specific embodiment, m is 8, n is 15, and X is CH₂.

In yet another specific embodiment, m is 9, n is 14, and X is O.

In yet another specific embodiment, m is 9, n is 14, and X is CH₂.

In yet another specific embodiment, m is 10, n is 13, and X is O.

In yet another specific embodiment, m is 10, n is 13, and X is CH₂.

In yet another specific embodiment, m is 11, n is 12, and X is O.

In yet another specific embodiment, m is 11, n is 12, and X is CH₂.

In yet another specific embodiment, m is 12, n is 11, and X is O.

In yet another specific embodiment, m is 12, n is 11, and X is CH₂.

In yet another specific embodiment, m is 13, n is 10, and X is O.

In yet another specific embodiment, m is 13, n is 10, and X is CH₂.

In yet another specific embodiment, m is 14, n is 9, and X is O.

In yet another specific embodiment, m is 14, n is 9, and X is CH₂.

In yet another specific embodiment, m is 15, n is 8, and X is O.

In yet another specific embodiment, m is 15, n is 8, and X is CH₂.

In yet another specific embodiment, m is 16, n is 7, and X is O.

In yet another specific embodiment, m is 16, n is 7, and X is CH₂.

In yet another specific embodiment, m is 17, n is 6, and X is O.

In yet another specific embodiment, m is 17, n is 6, and X is CH₂.

In yet another specific embodiment, m is 18, n is 5, and X is O.

In yet another specific embodiment, m is 18, n is 5, and X is CH₂.

In yet another specific embodiment, m is 19, n is 4, and X is O.

In yet another specific embodiment, m is 19, n is 4, and X is CH₂.

In yet another specific embodiment, m is 20, n is 3, and X is O.

In yet another specific embodiment, m is 20, n is 3, and X is CH₂.

In yet another specific embodiment, m is 21, n is 2, and X is O.

In yet another specific embodiment, m is 21, n is 2, and X is CH₂.

In yet another specific embodiment, m is 22, n is 1, and X is O.

In yet another specific embodiment, m is 22, n is 1, and X is CH₂.

In yet another specific embodiment, m is 23, n is 0, and X is O.

In yet another specific embodiment, m is 23, n is 0, and X is CH₂.

In another specific aspect, X, m and n in Formula (II-7) are as described in the First Specific Embodiment for Formula (I-5); and R and R¹ are as described above for Formula (II-7).

In another specific aspect, X, m and n in Formula (II-7) are as described in the Second Specific Embodiment for Formula (I-5); and R and R¹ are as described above for Formula (II-7).

In another specific aspect, X, m and n in Formula (II-7) are as described in the Third Specific Embodiment for Formula (I-5); and R and R¹ are as described above for Formula (II-7).

In another specific aspect, X, m and n in Formula (II-7) are as described in the Fourth Specific Embodiment for Formula (I-5); and R and R¹ are as described above for Formula (II-7).

In another specific aspect, X, m and n in Formula (II-7) are as described in the Fifth Specific Embodiment for Formula (I-5); and R and R¹ are as described above for Formula (II-7).

In another specific aspect, X, m and n in Formula (II-7) are as described in the Sixth Specific Embodiment for Formula (I-5); and R and R¹ are as described above for Formula (II-7).

Pharmaceutically acceptable salts of any one of the foregoing embodiments described in the 21^{st} aspect are also included.

In a 22^{nd} aspect, provided herein is a compound, or pharmaceutically acceptable salt thereof, having a structure provided in Formula (II-8),

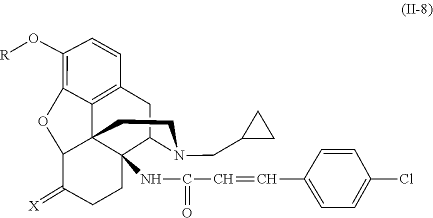

wherein:
X is O or CH₂;
R is —C(O)OR¹;
wherein R¹ is

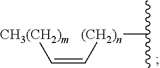

and
wherein m and n are integers between 0 and 23, and the sum of m and n is an integer between 6 and 23.

In one specific embodiment, m is 0, n is 23, and X is O.

In another specific embodiment, m is 0, n is 23, and X is CH₂.

In yet another specific embodiment, m is 1, n is 22, and X is O.

In yet another specific embodiment, m is 1, n is 22, and X is CH₂.

In yet another specific embodiment, m is 2, n is 21, and X is O.

In yet another specific embodiment, m is 2, n is 21, and X is CH₂.

In yet another specific embodiment, m is 3, n is 20, and X is O.

In yet another specific embodiment, m is 3, n is 20, and X is CH₂.

In yet another specific embodiment, m is 4, n is 19, and X is O.

In yet another specific embodiment, m is 4, n is 19, and X is CH₂.

In yet another specific embodiment, m is 5, n is 18, and X is O.

In yet another specific embodiment, m is 5, n is 18, and X is CH₂.

In yet another specific embodiment, m is 6, n is 17, and X is O.

In yet another specific embodiment, m is 6, n is 17, and X is CH₂.

In yet another specific embodiment, m is 7, n is 16, and X is O.

In yet another specific embodiment, m is 7, n is 16, and X is CH₂.

In yet another specific embodiment, m is 8, n is 15, and X is O.

In yet another specific embodiment, m is 8, n is 15, and X is CH₂.

In yet another specific embodiment, m is 9, n is 14, and X is O.

In yet another specific embodiment, m is 9, n is 14, and X is CH₂.

In yet another specific embodiment, m is 10, n is 13, and X is O.

In yet another specific embodiment, m is 10, n is 13, and X is CH₂.

In yet another specific embodiment, m is 11, n is 12, and X is O.

In yet another specific embodiment, m is 11, n is 12, and X is CH₂.

In yet another specific embodiment, m is 12, n is 11, and X is O.

In yet another specific embodiment, m is 12, n is 11, and X is CH₂.

In yet another specific embodiment, m is 13, n is 10, and X is O.

In yet another specific embodiment, m is 13, n is 10, and X is CH₂.

In yet another specific embodiment, m is 14, n is 9, and X is O.

In yet another specific embodiment, m is 14, n is 9, and X is CH₂.

In yet another specific embodiment, m is 15, n is 8, and X is O.

In yet another specific embodiment, m is 15, n is 8, and X is CH₂.

In yet another specific embodiment, m is 16, n is 7, and X is O.

In yet another specific embodiment, m is 16, n is 7, and X is CH₂.

In yet another specific embodiment, m is 17, n is 6, and X is O.

In yet another specific embodiment, m is 17, n is 6, and X is CH₂.

In yet another specific embodiment, m is 18, n is 5, and X is O.

In yet another specific embodiment, m is 18, n is 5, and X is CH₂.

In yet another specific embodiment, m is 19, n is 4, and X is O.

In yet another specific embodiment, m is 19, n is 4, and X is CH₂.

In yet another specific embodiment, m is 20, n is 3, and X is O.

In yet another specific embodiment, m is 20, n is 3, and X is CH₂.

In yet another specific embodiment, m is 21, n is 2, and X is O.

In yet another specific embodiment, m is 21, n is 2, and X is CH₂.

In yet another specific embodiment, m is 22, n is 1, and X is O.

In yet another specific embodiment, m is 22, n is 1, and X is CH₂.

In yet another specific embodiment, m is 23, n is 0, and X is O.

In yet another specific embodiment, m is 23, n is 0, and X is CH₂.

In another specific aspect, X, m and n in Formula (II-8) are as described in the First Specific Embodiment for Formula (I-5); and R and R¹ are as described above for Formula (II-8).

In another specific aspect, X, m and n in Formula (II-8) are as described in the Second Specific Embodiment for Formula (I-5); and R and R¹ are as described above for Formula (II-8).

In another specific aspect, X, m and n in Formula (II-8) are as described in the Third Specific Embodiment for Formula (I-5); and R and R¹ are as described above for Formula (II-8).

In another specific aspect, X, m and n in Formula (II-8) are as described in the Fourth Specific Embodiment for Formula (I-5); and R and R¹ are as described above for Formula (II-8).

In another specific aspect, X, m and n in Formula (II-8) are as described in the Fifth Specific Embodiment for Formula (I-5); and R and R¹ are as described above for Formula (II-8).

In another specific aspect, X, m and n in Formula (II-8) are as described in the Sixth Specific Embodiment for Formula (I-5); and R and R¹ are as described above for Formula (II-8).

Pharmaceutically acceptable salts of any one of the foregoing embodiments described in the 22^{nd} aspect are also included.

In a 23^{rd} aspect, provided herein is a compound, or pharmaceutically acceptable salt thereof, having a structure provided in Formula (II-9),

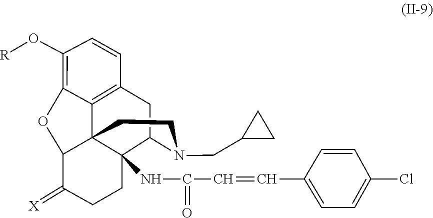

wherein:
X is O or CH₂;
R is —CH₂OC(O)R¹;
wherein R¹ is

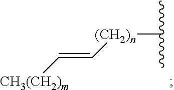

and
wherein m and n are integers between 0 and 23, and the sum of m and n is an integer between 6 and 23.

In one specific embodiment, m is 0, n is 23, and X is O.

In another specific embodiment, m is 0, n is 23, and X is CH₂.

In yet another specific embodiment, m is 1, n is 22, and X is O.

In yet another specific embodiment, m is 1, n is 22, and X is CH₂.

In yet another specific embodiment, m is 2, n is 21, and X is O.

In yet another specific embodiment, m is 2, n is 21, and X is CH₂.

In yet another specific embodiment, m is 3, n is 20, and X is O.

In yet another specific embodiment, m is 3, n is 20, and X is CH₂.

In yet another specific embodiment, m is 4, n is 19, and X is O.

In yet another specific embodiment, m is 4, n is 19, and X is CH₂.

In yet another specific embodiment, m is 5, n is 18, and X is O.

In yet another specific embodiment, m is 5, n is 18, and X is CH₂.

In yet another specific embodiment, m is 6, n is 17, and X is O.

In yet another specific embodiment, m is 6, n is 17, and X is CH₂.

In yet another specific embodiment, m is 7, n is 16, and X is O.

In yet another specific embodiment, m is 7, n is 16, and X is CH₂.

In yet another specific embodiment, m is 8, n is 15, and X is O.

In yet another specific embodiment, m is 8, n is 15, and X is CH₂.

In yet another specific embodiment, m is 9, n is 14, and X is O.

In yet another specific embodiment, m is 9, n is 14, and X is CH₂.

In yet another specific embodiment, m is 10, n is 13, and X is O.

In yet another specific embodiment, m is 10, n is 13, and X is CH₂.

In yet another specific embodiment, m is 11, n is 12, and X is O.

In yet another specific embodiment, m is 11, n is 12, and X is CH₂.

In yet another specific embodiment, m is 12, n is 11, and X is O.

In yet another specific embodiment, m is 12, n is 11, and X is CH₂.

In yet another specific embodiment, m is 13, n is 10, and X is O.

In yet another specific embodiment, m is 13, n is 10, and X is CH₂.

In yet another specific embodiment, m is 14, n is 9, and X is O.

In yet another specific embodiment, m is 14, n is 9, and X is CH₂.

In yet another specific embodiment, m is 15, n is 8, and X is O.

In yet another specific embodiment, m is 15, n is 8, and X is CH₂.

In yet another specific embodiment, m is 16, n is 7, and X is O.

In yet another specific embodiment, m is 16, n is 7, and X is CH₂.

In yet another specific embodiment, m is 17, n is 6, and X is O.

In yet another specific embodiment, m is 17, n is 6, and X is CH₂.

In yet another specific embodiment, m is 18, n is 5, and X is O.

In yet another specific embodiment, m is 18, n is 5, and X is CH₂.

In yet another specific embodiment, m is 19, n is 4, and X is O.

In yet another specific embodiment, m is 19, n is 4, and X is CH₂.

In yet another specific embodiment, m is 20, n is 3, and X is O.

In yet another specific embodiment, m is 20, n is 3, and X is CH₂.

In yet another specific embodiment, m is 21, n is 2, and X is O.

In yet another specific embodiment, m is 21, n is 2, and X is CH₂.

In yet another specific embodiment, m is 22, n is 1, and X is O.

In yet another specific embodiment, m is 22, n is 1, and X is CH₂.

In yet another specific embodiment, m is 23, n is 0, and X is O.

In yet another specific embodiment, m is 23, n is 0, and X is CH₂.

In another specific aspect, X, m and n in Formula (II-9) are as described in the First Specific Embodiment for Formula (I-5); and R and R¹ are as described above for Formula (II-9).

In another specific aspect, X, m and n in Formula (II-9) are as described in the Second Specific Embodiment for Formula (I-5); and R and R¹ are as described above for Formula (II-9).

In another specific aspect, X, m and n in Formula (II-9) are as described in the Third Specific Embodiment for Formula (I-5); and R and R¹ are as described above for Formula (II-9).

In another specific aspect, X, m and n in Formula (II-9) are as described in the Fourth Specific Embodiment for Formula (I-5); and R and R¹ are as described above for Formula (II-9).

In another specific aspect, X, m and n in Formula (II-9) are as described in the Fifth Specific Embodiment for Formula (I-5); and R and R¹ are as described above for Formula (II-9).

In another specific aspect, X, m and n in Formula (II-9) are as described in the Sixth Specific Embodiment for Formula (I-5); and R and R¹ are as described above for Formula (II-9).

Pharmaceutically acceptable salts of any one of the foregoing embodiments described in the 23^{rd} aspect are also included.

In a 24^{th} aspect, provided herein is a compound, or pharmaceutically acceptable salt thereof, having a structure provided in Formula (II-10),

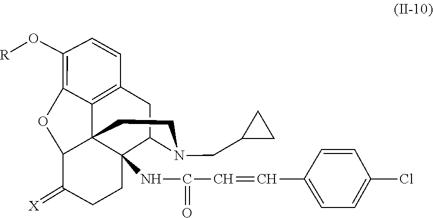

wherein:
X is O or CH₂;
R is —CH₂OC(O)R¹;
wherein R¹ is

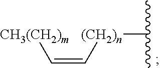

and
wherein m and n are integers between 0 and 23, and the sum of m and n is an integer between 6 and 23.

In one specific embodiment, m is 0, n is 23, and X is O.

In another specific embodiment, m is 0, n is 23, and X is CH₂.

In yet another specific embodiment, m is 1, n is 22, and X is O.

In yet another specific embodiment, m is 1, n is 22, and X is CH₂.

In yet another specific embodiment, m is 2, n is 21, and X is O.

In yet another specific embodiment, m is 2, n is 21, and X is CH₂.

In yet another specific embodiment, m is 3, n is 20, and X is O.

In yet another specific embodiment, m is 3, n is 20, and X is CH₂.

In yet another specific embodiment, m is 4, n is 19, and X is O.

In yet another specific embodiment, m is 4, n is 19, and X is CH₂.

In yet another specific embodiment, m is 5, n is 18, and X is O.

In yet another specific embodiment, m is 5, n is 18, and X is CH₂.

In yet another specific embodiment, m is 6, n is 17, and X is O.

In yet another specific embodiment, m is 6, n is 17, and X is CH₂.

In yet another specific embodiment, m is 7, n is 16, and X is O.

In yet another specific embodiment, m is 7, n is 16, and X is CH₂.

In yet another specific embodiment, m is 8, n is 15, and X is O.

In yet another specific embodiment, m is 8, n is 15, and X is CH₂.

In yet another specific embodiment, m is 9, n is 14, and X is O.

In yet another specific embodiment, m is 9, n is 14, and X is CH₂.

In yet another specific embodiment, m is 10, n is 13, and X is O.

In yet another specific embodiment, m is 10, n is 13, and X is CH₂.

In yet another specific embodiment, m is 11, n is 12, and X is O.

In yet another specific embodiment, m is 11, n is 12, and X is CH₂.

In yet another specific embodiment, m is 12, n is 11, and X is O.

In yet another specific embodiment, m is 12, n is 11, and X is CH₂.

In yet another specific embodiment, m is 13, n is 10, and X is O.

In yet another specific embodiment, m is 13, n is 10, and X is CH₂.

In yet another specific embodiment, m is 14, n is 9, and X is O.

In yet another specific embodiment, m is 14, n is 9, and X is CH₂.

In yet another specific embodiment, m is 15, n is 8, and X is O.

In yet another specific embodiment, m is 15, n is 8, and X is CH₂.

In yet another specific embodiment, m is 16, n is 7, and X is O.

In yet another specific embodiment, m is 16, n is 7, and X is CH₂.

In yet another specific embodiment, m is 17, n is 6, and X is O.

In yet another specific embodiment, m is 17, n is 6, and X is CH₂.

In yet another specific embodiment, m is 18, n is 5, and X is O.

In yet another specific embodiment, m is 18, n is 5, and X is CH₂.

In yet another specific embodiment, m is 19, n is 4, and X is O.

In yet another specific embodiment, m is 19, n is 4, and X is CH₂.

In yet another specific embodiment, m is 20, n is 3, and X is O.

In yet another specific embodiment, m is 20, n is 3, and X is CH₂.

In yet another specific embodiment, m is 21, n is 2, and X is O.

In yet another specific embodiment, m is 21, n is 2, and X is CH₂.

In yet another specific embodiment, m is 22, n is 1, and X is O.

In yet another specific embodiment, m is 22, n is 1, and X is CH₂.

In yet another specific embodiment, m is 23, n is 0, and X is O.

In yet another specific embodiment, m is 23, n is 0, and X is CH₂.

In another specific aspect, X, m and n in Formula (II-10) are as described in the First Specific Embodiment for Formula (I-5); and R and R¹ are as described above for Formula (II-10).

In another specific aspect, X, m and n in Formula (II-10) are as described in the Second Specific Embodiment for Formula (I-5); and R and R¹ are as described above for Formula (II-10).

In another specific aspect, X, m and n in Formula (II-10) are as described in the Third Specific Embodiment for Formula (I-5); and R and R¹ are as described above for Formula (II-10).

In another specific aspect, X, m and n in Formula (II-10) are as described in the Fourth Specific Embodiment for Formula (I-5); and R and R¹ are as described above for Formula (II-10).

In another specific aspect, X, m and n in Formula (II-10) are as described in the Fifth Specific Embodiment for Formula (I-5); and R and R¹ are as described above for Formula (II-10).

In another specific aspect, X, m and n in Formula (II-10) are as described in the Sixth Specific Embodiment for Formula (I-5); and R and R¹ are as described above for Formula (II-10).

Pharmaceutically acceptable salts of any one of the foregoing embodiments described in the 24^{th} aspect are also included.

In a 25^{th} aspect, provided herein is a compound, or pharmaceutically acceptable salt thereof, having a structure provided in Formula (II-11),

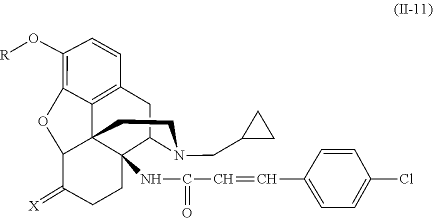

wherein:
X is O or CH₂;
R is —CH₂OC(O)OR¹;
wherein R¹ is

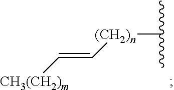

and
wherein m and n are integers between 0 and 23, and the sum of m and n is an integer between 6 and 23.

In one specific embodiment, m is 0, n is 23, and X is O.

In another specific embodiment, m is 0, n is 23, and X is CH₂.

In yet another specific embodiment, m is 1, n is 22, and X is O.

In yet another specific embodiment, m is 1, n is 22, and X is CH₂.

In yet another specific embodiment, m is 2, n is 21, and X is O.

In yet another specific embodiment, m is 2, n is 21, and X is CH₂.

In yet another specific embodiment, m is 3, n is 20, and X is O.

In yet another specific embodiment, m is 3, n is 20, and X is CH₂.

In yet another specific embodiment, m is 4, n is 19, and X is O.

In yet another specific embodiment, m is 4, n is 19, and X is CH₂.

In yet another specific embodiment, m is 5, n is 18, and X is O.

In yet another specific embodiment, m is 5, n is 18, and X is CH₂.

In yet another specific embodiment, m is 6, n is 17, and X is O.

In yet another specific embodiment, m is 6, n is 17, and X is CH₂.

In yet another specific embodiment, m is 7, n is 16, and X is O.

In yet another specific embodiment, m is 7, n is 16, and X is CH₂.

In yet another specific embodiment, m is 8, n is 15, and X is O.

In yet another specific embodiment, m is 8, n is 15, and X is CH₂.

In yet another specific embodiment, m is 9, n is 14, and X is O.

In yet another specific embodiment, m is 9, n is 14, and X is CH₂.

In yet another specific embodiment, m is 10, n is 13, and X is O.

In yet another specific embodiment, m is 10, n is 13, and X is CH₂.

In yet another specific embodiment, m is 11, n is 12, and X is O.

In yet another specific embodiment, m is 11, n is 12, and X is CH₂.

In yet another specific embodiment, m is 12, n is 11, and X is O.

In yet another specific embodiment, m is 12, n is 11, and X is CH₂.

In yet another specific embodiment, m is 13, n is 10, and X is O.

In yet another specific embodiment, m is 13, n is 10, and X is CH₂.

In yet another specific embodiment, m is 14, n is 9, and X is O.

In yet another specific embodiment, m is 14, n is 9, and X is CH₂.

In yet another specific embodiment, m is 15, n is 8, and X is O.

In yet another specific embodiment, m is 15, n is 8, and X is CH₂.

In yet another specific embodiment, m is 16, n is 7, and X is O.

In yet another specific embodiment, m is 16, n is 7, and X is CH₂.

In yet another specific embodiment, m is 17, n is 6, and X is O.

In yet another specific embodiment, m is 17, n is 6, and X is CH₂.

In yet another specific embodiment, m is 18, n is 5, and X is O.

In yet another specific embodiment, m is 18, n is 5, and X is CH₂.

In yet another specific embodiment, m is 19, n is 4, and X is O.

In yet another specific embodiment, m is 19, n is 4, and X is CH₂.

In yet another specific embodiment, m is 20, n is 3, and X is O.

In yet another specific embodiment, m is 20, n is 3, and X is CH₂.

In yet another specific embodiment, m is 21, n is 2, and X is O.

In yet another specific embodiment, m is 21, n is 2, and X is CH₂.

In yet another specific embodiment, m is 22, n is 1, and X is O.

In yet another specific embodiment, m is 22, n is 1, and X is CH₂.

In yet another specific embodiment, m is 23, n is 0, and X is O.

In yet another specific embodiment, m is 23, n is 0, and X is CH₂.

In another specific aspect, X, m and n in Formula (II-11) are as described in the First Specific Embodiment for Formula (I-5); and R and R¹ are as described above for Formula (II-11).

In another specific aspect, X, m and n in Formula (II-11) are as described in the Second Specific Embodiment for Formula (I-5); and R and R¹ are as described above for Formula (II-11).

In another specific aspect, X, m and n in Formula (II-11) are as described in the Third Specific Embodiment for Formula (I-5); and R and R¹ are as described above for Formula (II-11).

In another specific aspect, X, m and n in Formula (II-11) are as described in the Fourth Specific Embodiment for Formula (I-5); and R and R¹ are as described above for Formula (II-11).

In another specific aspect, X, m and n in Formula (II-11) are as described in the Fifth Specific Embodiment for Formula (I-5); and R and R¹ are as described above for Formula (II-11).

In another specific aspect, X, m and n in Formula (II-11) are as described in the Sixth Specific Embodiment for Formula (I-5); and R and R¹ are as described above for Formula (II-11).

Pharmaceutically acceptable salts of any one of the foregoing embodiments described in the 25^{th} aspect are also included.

In a 26^{th} aspect, provided herein is a compound, or pharmaceutically acceptable salt thereof, having a structure provided in Formula (II-12),

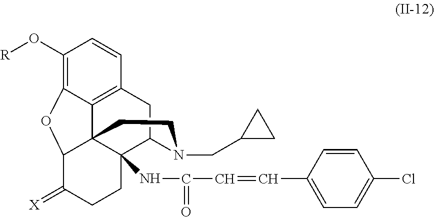

wherein:
X is O or CH₂;
R is —CH₂OC(O)OR¹;
wherein R¹ is

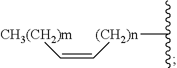

and
wherein m and n are integers between 0 and 23, and the sum of m and n is an integer between 6 and 23.

In one specific embodiment, m is 0, n is 23, and X is O.

In another specific embodiment, m is 0, n is 23, and X is CH₂.

In yet another specific embodiment, m is 1, n is 22, and X is O.

In yet another specific embodiment, m is 1, n is 22, and X is CH₂.

In yet another specific embodiment, m is 2, n is 21, and X is O.

In yet another specific embodiment, m is 2, n is 21, and X is CH₂.

In yet another specific embodiment, m is 3, n is 20, and X is O.

In yet another specific embodiment, m is 3, n is 20, and X is CH₂.

In yet another specific embodiment, m is 4, n is 19, and X is O.

In yet another specific embodiment, m is 4, n is 19, and X is CH₂.

In yet another specific embodiment, m is 5, n is 18, and X is O.

In yet another specific embodiment, m is 5, n is 18, and X is CH₂.

In yet another specific embodiment, m is 6, n is 17, and X is O.

In yet another specific embodiment, m is 6, n is 17, and X is CH₂.

In yet another specific embodiment, m is 7, n is 16, and X is O.

In yet another specific embodiment, m is 7, n is 16, and X is CH₂.

In yet another specific embodiment, m is 8, n is 15, and X is O.

In yet another specific embodiment, m is 8, n is 15, and X is CH₂.

In yet another specific embodiment, m is 9, n is 14, and X is O.

In yet another specific embodiment, m is 9, n is 14, and X is CH₂.

In yet another specific embodiment, m is 10, n is 13, and X is O.

In yet another specific embodiment, m is 10, n is 13, and X is CH₂.

In yet another specific embodiment, m is 11, n is 12, and X is O.

In yet another specific embodiment, m is 11, n is 12, and X is CH₂.

In yet another specific embodiment, m is 12, n is 11, and X is O.

In yet another specific embodiment, m is 12, n is 11, and X is CH₂.

In yet another specific embodiment, m is 13, n is 10, and X is O.

In yet another specific embodiment, m is 13, n is 10, and X is CH₂.

In yet another specific embodiment, m is 14, n is 9, and X is O.

In yet another specific embodiment, m is 14, n is 9, and X is CH₂.

In yet another specific embodiment, m is 15, n is 8, and X is O.

In yet another specific embodiment, m is 15, n is 8, and X is CH₂.

In yet another specific embodiment, m is 16, n is 7, and X is O.

In yet another specific embodiment, m is 16, n is 7, and X is CH₂.

In yet another specific embodiment, m is 17, n is 6, and X is O.

In yet another specific embodiment, m is 17, n is 6, and X is CH₂.

In yet another specific embodiment, m is 18, n is 5, and X is O.

In yet another specific embodiment, m is 18, n is 5, and X is CH₂.

In yet another specific embodiment, m is 19, n is 4, and X is O.

In yet another specific embodiment, m is 19, n is 4, and X is CH₂.

In yet another specific embodiment, m is 20, n is 3, and X is O.

In yet another specific embodiment, m is 20, n is 3, and X is CH₂.

In yet another specific embodiment, m is 21, n is 2, and X is O.

In yet another specific embodiment, m is 21, n is 2, and X is CH₂.

In yet another specific embodiment, m is 22, n is 1, and X is O.

In yet another specific embodiment, m is 22, n is 1, and X is CH₂.

In yet another specific embodiment, m is 23, n is 0, and X is O.

In yet another specific embodiment, m is 23, n is 0, and X is CH₂.

In another specific aspect, X, m and n in Formula (II-12) are as described in the First Specific Embodiment for Formula (I-5); and R and R¹ are as described above for Formula (II-12).

In another specific aspect, X, m and n in Formula (II-12) are as described in the Second Specific Embodiment for Formula (I-5); and R and R¹ are as described above for Formula (II-12).

In another specific aspect, X, m and n in Formula (II-12) are as described in the Third Specific Embodiment for Formula (I-5); and R and R¹ are as described above for Formula (II-12).

In another specific aspect, X, m and n in Formula (II-12) are as described in the Fourth Specific Embodiment for Formula (I-5); and R and R¹ are as described above for Formula (II-12).

In another specific aspect, X, m and n in Formula (II-12) are as described in the Fifth Specific Embodiment for Formula (I-5); and R and R¹ are as described above for Formula (II-12).

In another specific aspect, X, m and n in Formula (II-12) are as described in the Sixth Specific Embodiment for Formula (I-5); and R and R¹ are as described above for Formula (II-12).

Pharmaceutically acceptable salts of any one of the foregoing embodiments described in the 26^{th} aspect are also included.

In a 27^{th} aspect, provided herein is a compound, or pharmaceutically acceptable salt thereof, having a structure provided in Formula (III):

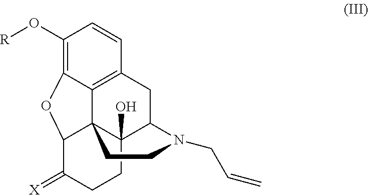

wherein:
X is O or CH₂;
R is selected from the group consisting of:
   (a) —C(O)R¹,
   (b) —C(O)OR¹,
   (c) —CH₂OC(O)R¹,
   (d) —CH₂OC(O)OR¹, and
   (e) —CH₂OR¹;
each instance of R¹ is independently —(CH₂)_{q}CH₃,

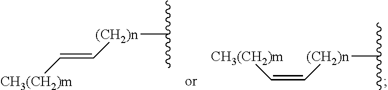

m and n are integers between 0 and 23, and the sum of m and n is an integer between 6 and 23; and
q is an integer between 0 and 25.

In a 28^{th} aspect, provided herein is a compound, or pharmaceutically acceptable salt thereof, having a structure provided in Formula (III-1),

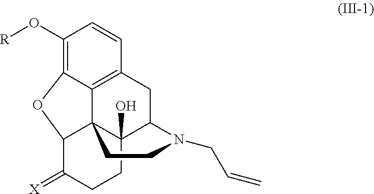

wherein:
X is O or CH₂;
R is —C(O)R¹;
wherein R¹ is —(CH₂)_{q}CH₃; and
wherein q is an integer between 0 and 25.

In one specific embodiment, q is 0 and X is O.

In another specific embodiment, q is 0 and X is CH₂.

In yet another specific embodiment, q is 1 and X is O.

In yet another specific embodiment, q is 1 and X is CH₂.

In yet another specific embodiment, q is 2 and X is O.

In yet another specific embodiment, q is 2 and X is CH₂.

In yet another specific embodiment, q is 3 and X is O.

In yet another specific embodiment, q is 3 and X is CH₂.

In yet another specific embodiment, q is 4 and X is O.

In yet another specific embodiment, q is 4 and X is CH₂.

In yet another specific embodiment, q is 5 and X is O.

In yet another specific embodiment, q is 5 and X is CH₂.

In yet another specific embodiment, q is 6 and X is O.

In yet another specific embodiment, q is 6 and X is CH₂.

In yet another specific embodiment, q is 7 and X is O.

In yet another specific embodiment, q is 7 and X is CH₂.

In yet another specific embodiment, q is 8 and X is O.

In yet another specific embodiment, q is 8 and X is CH₂.

In yet another specific embodiment, q is 9 and X is O.

In yet another specific embodiment, q is 9 and X is CH₂.

In yet another specific embodiment, q is 10 and X is O.

In yet another specific embodiment, q is 10 and X is CH₂.

In yet another specific embodiment, q is 11 and X is O.

In yet another specific embodiment, q is 11 and X is CH₂.

In yet another specific embodiment, q is 12 and X is O.

In yet another specific embodiment, q is 12 and X is CH₂.

In yet another specific embodiment, q is 13 and X is O.

In yet another specific embodiment, q is 13 and X is CH₂.

In yet another specific embodiment, q is 14 and X is O.

In yet another specific embodiment, q is 14 and X is CH₂.

In yet another specific embodiment, q is 15 and X is O.

In yet another specific embodiment, q is 15 and X is CH₂.

In yet another specific embodiment, q is 16 and X is O.

In yet another specific embodiment, q is 16 and X is CH₂.

In yet another specific embodiment, q is 17 and X is O.

In yet another specific embodiment, q is 17 and X is CH₂.

In yet another specific embodiment, q is 18 and X is O.

In yet another specific embodiment, q is 18 and X is CH₂.

In yet another specific embodiment, q is 19 and X is O.

In yet another specific embodiment, q is 19 and X is CH₂.

In yet another specific embodiment, q is 20 and X is O.

In yet another specific embodiment, q is 20 and X is CH₂.

In yet another specific embodiment, q is 21 and X is O.

In yet another specific embodiment, q is 21 and X is CH₂.

In yet another specific embodiment, q is 22 and X is O.

In yet another specific embodiment, q is 22 and X is CH₂.

In yet another specific embodiment, q is 23 and X is O.

In yet another specific embodiment, q is 23 and X is CH₂.

In yet another specific embodiment, q is 24 and X is O.

In yet another specific embodiment, q is 24 and X is CH₂.

In yet another specific embodiment, q is 25 and X is O.

In yet another specific embodiment, q is 25 and X is CH₂.

Pharmaceutically acceptable salts of any one of the foregoing embodiments described in the 28^{th} aspect are also included.

In a 29^{th} aspect, provided herein is a compound, or pharmaceutically acceptable salt thereof having a structure provided in Formula (III-2),

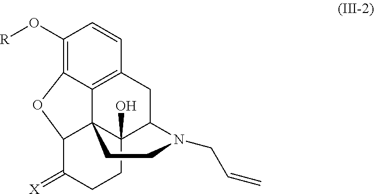

wherein:
X is O or CH₂;
R is —C(O)OR¹;
wherein R¹ is —(CH₂)_{q}CH₃; and
wherein q is an integer between 0 and 25.

In one specific embodiment, q is 0 and X is O.

In another specific embodiment, q is 0 and X is CH₂.

In yet another specific embodiment, q is 1 and X is O.

In yet another specific embodiment, q is 1 and X is CH₂.

In yet another specific embodiment, q is 2 and X is O.

In yet another specific embodiment, q is 2 and X is CH₂.

In yet another specific embodiment, q is 3 and X is O.

In yet another specific embodiment, q is 3 and X is CH₂.

In yet another specific embodiment, q is 4 and X is O.

In yet another specific embodiment, q is 4 and X is CH₂.

In yet another specific embodiment, q is 5 and X is O.

In yet another specific embodiment, q is 5 and X is CH₂.

In yet another specific embodiment, q is 6 and X is O.

In yet another specific embodiment, q is 6 and X is CH₂.

In yet another specific embodiment, q is 7 and X is O.

In yet another specific embodiment, q is 7 and X is CH₂.

In yet another specific embodiment, q is 8 and X is O.

In yet another specific embodiment, q is 8 and X is CH₂.

In yet another specific embodiment, q is 9 and X is O.

In yet another specific embodiment, q is 9 and X is CH₂.

In yet another specific embodiment, q is 10 and X is O.

In yet another specific embodiment, q is 10 and X is CH₂.

In yet another specific embodiment, q is 11 and X is O.

In yet another specific embodiment, q is 11 and X is CH₂.

In yet another specific embodiment, q is 12 and X is O.

In yet another specific embodiment, q is 12 and X is CH₂.

In yet another specific embodiment, q is 13 and X is O.

In yet another specific embodiment, q is 13 and X is CH₂.

In yet another specific embodiment, q is 14 and X is O.

In yet another specific embodiment, q is 14 and X is CH₂.

In yet another specific embodiment, q is 15 and X is O.

In yet another specific embodiment, q is 15 and X is CH₂.

In yet another specific embodiment, q is 16 and X is O.

In yet another specific embodiment, q is 16 and X is CH₂.

In yet another specific embodiment, q is 17 and X is O.

In yet another specific embodiment, q is 17 and X is CH₂.

In yet another specific embodiment, q is 18 and X is O.

In yet another specific embodiment, q is 18 and X is CH₂.

In yet another specific embodiment, q is 19 and X is O.

In yet another specific embodiment, q is 19 and X is CH₂.

In yet another specific embodiment, q is 20 and X is O.

In yet another specific embodiment, q is 20 and X is CH₂.

In yet another specific embodiment, q is 21 and X is O.

In yet another specific embodiment, q is 21 and X is CH₂.

In yet another specific embodiment, q is 22 and X is O.

In yet another specific embodiment, q is 22 and X is CH₂.

In yet another specific embodiment, q is 23 and X is O.

In yet another specific embodiment, q is 23 and X is CH₂.

In yet another specific embodiment, q is 24 and X is O.

In yet another specific embodiment, q is 24 and X is CH₂.

In yet another specific embodiment, q is 25 and X is O.

In yet another specific embodiment, q is 25 and X is CH₂.

Pharmaceutically acceptable salts of any one of the foregoing embodiments described in the 29^{th} aspect are also included.

In a 30^{th} aspect, provided herein is a compound, or pharmaceutically acceptable salt thereof, having a structure provided in Formula (III-3),

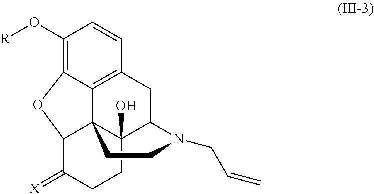

wherein:
X is O or CH₂;
R is —CH₂OC(O)R¹;
wherein R¹ is —(CH₂)_{q}CH₃; and
wherein q is an integer between 0 and 25.

In one specific embodiment, q is 0 and X is O.

In another specific embodiment, q is 0 and X is CH₂.

In yet another specific embodiment, q is 1 and X is O.

In yet another specific embodiment, q is 1 and X is CH₂.

In yet another specific embodiment, q is 2 and X is O.

In yet another specific embodiment, q is 2 and X is CH₂.

In yet another specific embodiment, q is 3 and X is O.

In yet another specific embodiment, q is 3 and X is CH₂.

In yet another specific embodiment, q is 4 and X is O.

In yet another specific embodiment, q is 4 and X is CH₂.

In yet another specific embodiment, q is 5 and X is O.

In yet another specific embodiment, q is 5 and X is CH₂.

In yet another specific embodiment, q is 6 and X is O.

In yet another specific embodiment, q is 6 and X is CH₂.

In yet another specific embodiment, q is 7 and X is O.

In yet another specific embodiment, q is 7 and X is CH₂.

In yet another specific embodiment, q is 8 and X is O.

In yet another specific embodiment, q is 8 and X is CH₂.

In yet another specific embodiment, q is 9 and X is O.

In yet another specific embodiment, q is 9 and X is CH₂.

In yet another specific embodiment, q is 10 and X is O.

In yet another specific embodiment, q is 10 and X is CH₂.

In yet another specific embodiment, q is 11 and X is O.

In yet another specific embodiment, q is 11 and X is CH₂.

In yet another specific embodiment, q is 12 and X is O.

In yet another specific embodiment, q is 12 and X is CH₂.

In yet another specific embodiment, q is 13 and X is O.

In yet another specific embodiment, q is 13 and X is CH₂.

In yet another specific embodiment, q is 14 and X is O.

In yet another specific embodiment, q is 14 and X is CH₂.

In yet another specific embodiment, q is 15 and X is O.

In yet another specific embodiment, q is 15 and X is CH₂.

In yet another specific embodiment, q is 16 and X is O.

In yet another specific embodiment, q is 16 and X is CH₂.

In yet another specific embodiment, q is 17 and X is O.

In yet another specific embodiment, q is 17 and X is CH₂.

In yet another specific embodiment, q is 18 and X is O.

In yet another specific embodiment, q is 18 and X is CH₂.

In yet another specific embodiment, q is 19 and X is O.

In yet another specific embodiment, q is 19 and X is CH₂.

In yet another specific embodiment, q is 20 and X is O.

In yet another specific embodiment, q is 20 and X is CH₂.

In yet another specific embodiment, q is 21 and X is O.

In yet another specific embodiment, q is 21 and X is CH₂.

In yet another specific embodiment, q is 22 and X is O.

In yet another specific embodiment, q is 22 and X is CH₂.

In yet another specific embodiment, q is 23 and X is O.

In yet another specific embodiment, q is 23 and X is CH₂.

In yet another specific embodiment, q is 24 and X is O.

In yet another specific embodiment, q is 24 and X is CH₂.

In yet another specific embodiment, q is 25 and X is O.

In yet another specific embodiment, q is 25 and X is CH₂.

Pharmaceutically acceptable salts of any one of the foregoing embodiments described in the 30^{th} aspect are also included.

In a 31^{st} aspect, provided herein is a compound, or pharmaceutically acceptable salt thereof, having a structure provided in Formula (III-4),

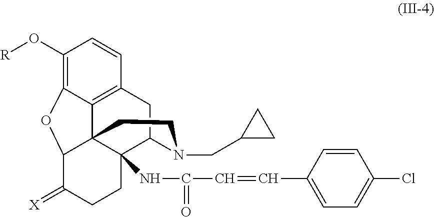

wherein:
X is O or CH₂;
R is —CH₂OC(O)OR¹;
wherein R¹ is —(CH₂)_{q}CH₃; and
wherein q is an integer between 0 and 25.

In one specific embodiment, q is 0 and X is O.

In another specific embodiment, q is 0 and X is CH₂.

In yet another specific embodiment, q is 1 and X is O.

In yet another specific embodiment, q is 1 and X is CH₂.

In yet another specific embodiment, q is 2 and X is O.

In yet another specific embodiment, q is 2 and X is CH₂.

In yet another specific embodiment, q is 3 and X is O.

In yet another specific embodiment, q is 3 and X is CH₂.

In yet another specific embodiment, q is 4 and X is O.

In yet another specific embodiment, q is 4 and X is CH₂.

In yet another specific embodiment, q is 5 and X is O.

In yet another specific embodiment, q is 5 and X is CH₂.

In yet another specific embodiment, q is 6 and X is O.

In yet another specific embodiment, q is 6 and X is CH₂.

In yet another specific embodiment, q is 7 and X is O.

In yet another specific embodiment, q is 7 and X is CH₂.

In yet another specific embodiment, q is 8 and X is O.

In yet another specific embodiment, q is 8 and X is CH₂.

In yet another specific embodiment, q is 9 and X is O.

In yet another specific embodiment, q is 9 and X is CH₂.

In yet another specific embodiment, q is 10 and X is O.

In yet another specific embodiment, q is 10 and X is CH₂.

In yet another specific embodiment, q is 11 and X is O.

In yet another specific embodiment, q is 11 and X is CH₂.

In yet another specific embodiment, q is 12 and X is O.

In yet another specific embodiment, q is 12 and X is CH₂.

In yet another specific embodiment, q is 13 and X is O.

In yet another specific embodiment, q is 13 and X is CH₂.

In yet another specific embodiment, q is 14 and X is O.

In yet another specific embodiment, q is 14 and X is CH₂.

In yet another specific embodiment, q is 15 and X is O.

In yet another specific embodiment, q is 15 and X is CH₂.

In yet another specific embodiment, q is 16 and X is O.

In yet another specific embodiment, q is 16 and X is CH₂.

In yet another specific embodiment, q is 17 and X is O.

In yet another specific embodiment, q is 17 and X is CH₂.

In yet another specific embodiment, q is 18 and X is O.

In yet another specific embodiment, q is 18 and X is CH₂.

In yet another specific embodiment, q is 19 and X is O.

In yet another specific embodiment, q is 19 and X is CH₂.

In yet another specific embodiment, q is 20 and X is O.

In yet another specific embodiment, q is 20 and X is CH₂.

In yet another specific embodiment, q is 21 and X is O.

In yet another specific embodiment, q is 21 and X is CH₂.

In yet another specific embodiment, q is 22 and X is O.

In yet another specific embodiment, q is 22 and X is CH₂.

In yet another specific embodiment, q is 23 and X is O.

In yet another specific embodiment, q is 23 and X is CH₂.

In yet another specific embodiment, q is 24 and X is O.

In yet another specific embodiment, q is 24 and X is CH₂.

In yet another specific embodiment, q is 25 and X is O.

In yet another specific embodiment, q is 25 and X is CH₂.

Pharmaceutically acceptable salts of any one of the foregoing embodiments described in the 31^{st} aspect are also included.

In a 32^{nd} aspect, provided herein is a compound, or pharmaceutically acceptable salt thereof, having a structure provided in Formula (III-5),

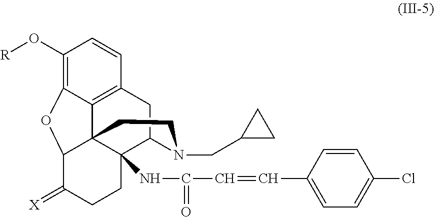

wherein:
X is O or CH₂;
R is —C(O)R¹;
wherein R¹ is

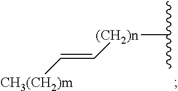

and
wherein m and n are integers between 0 and 23, and the sum of m and n is an integer between 6 and 23.

In one specific embodiment, m is 0, n is 23, and X is O.

In another specific embodiment, m is 0, n is 23, and X is CH₂.

In yet another specific embodiment, m is 1, n is 22, and X is O.

In yet another specific embodiment, m is 1, n is 22, and X is CH₂.

In yet another specific embodiment, m is 2, n is 21, and X is O.

In yet another specific embodiment, m is 2, n is 21, and X is CH₂.

In yet another specific embodiment, m is 3, n is 20, and X is O.

In yet another specific embodiment, m is 3, n is 20, and X is CH₂.

In yet another specific embodiment, m is 4, n is 19, and X is O.

In yet another specific embodiment, m is 4, n is 19, and X is CH₂.

In yet another specific embodiment, m is 5, n is 18, and X is O.

In yet another specific embodiment, m is 5, n is 18, and X is CH₂.

In yet another specific embodiment, m is 6, n is 17, and X is O.

In yet another specific embodiment, m is 6, n is 17, and X is CH₂.

In yet another specific embodiment, m is 7, n is 16, and X is O.

In yet another specific embodiment, m is 7, n is 16, and X is CH₂.

In yet another specific embodiment, m is 8, n is 15, and X is O.

In yet another specific embodiment, m is 8, n is 15, and X is CH₂.

In yet another specific embodiment, m is 9, n is 14, and X is O.

In yet another specific embodiment, m is 9, n is 14, and X is CH₂.

In yet another specific embodiment, m is 10, n is 13, and X is O.

In yet another specific embodiment, m is 10, n is 13, and X is CH₂.

In yet another specific embodiment, m is 11, n is 12, and X is O.

In yet another specific embodiment, m is 11, n is 12, and X is CH₂.

In yet another specific embodiment, m is 12, n is 11, and X is O.

In yet another specific embodiment, m is 12, n is 11, and X is CH₂.

In yet another specific embodiment, m is 13, n is 10, and X is O.

In yet another specific embodiment, m is 13, n is 10, and X is CH₂.

In yet another specific embodiment, m is 14, n is 9, and X is O.

In yet another specific embodiment, m is 14, n is 9, and X is CH₂.

In yet another specific embodiment, m is 15, n is 8, and X is O.

In yet another specific embodiment, m is 15, n is 8, and X is CH₂.

In yet another specific embodiment, m is 16, n is 7, and X is O.

In yet another specific embodiment, m is 16, n is 7, and X is CH₂.

In yet another specific embodiment, m is 17, n is 6, and X is O.

In yet another specific embodiment, m is 17, n is 6, and X is CH₂.

In yet another specific embodiment, m is 18, n is 5, and X is O.

In yet another specific embodiment, m is 18, n is 5, and X is CH₂.

In yet another specific embodiment, m is 19, n is 4, and X is O.

In yet another specific embodiment, m is 19, n is 4, and X is CH₂.

In yet another specific embodiment, m is 20, n is 3, and X is O.

In yet another specific embodiment, m is 20, n is 3, and X is CH₂.

In yet another specific embodiment, m is 21, n is 2, and X is O.

In yet another specific embodiment, m is 21, n is 2, and X is CH₂.

In yet another specific embodiment, m is 22, n is 1, and X is O.

In yet another specific embodiment, m is 22, n is 1, and X is CH₂.

In yet another specific embodiment, m is 23, n is 0, and X is O.

In yet another specific embodiment, m is 23, n is 0, and X is CH₂.

In another specific aspect, X, m and n in Formula (III-5) are as described in the First Specific Embodiment for Formula (I-5); and R and R¹ are as described above for Formula (III-5).

In another specific aspect, X, m and n in Formula (III-5) are as described in the Second Specific Embodiment for Formula (I-5); and R and R¹ are as described above for Formula (III-5).

In another specific aspect, X, m and n in Formula (III-5) are as described in the Third Specific Embodiment for Formula (I-5); and R and R¹ are as described above for Formula (III-5).

In another specific aspect, X, m and n in Formula (III-5) are as described in the Fourth Specific Embodiment for Formula (I-5); and R and R¹ are as described above for Formula (III-5).

In another specific aspect, X, m and n in Formula (III-5) are as described in the Fifth Specific Embodiment for Formula (I-5); and R and R¹ are as described above for Formula (III-5).

In another specific aspect, X, m and n in Formula (III-5) are as described in the Sixth Specific Embodiment for Formula (I-5); and R and R¹ are as described above for Formula (III-5).

Pharmaceutically acceptable salts of any one of the foregoing embodiments described in the 32^{nd} aspect are also included.

In a 33^{rd} aspect, provided herein is a compound, or pharmaceutically acceptable salt thereof, having a structure provided in Formula (III-6),

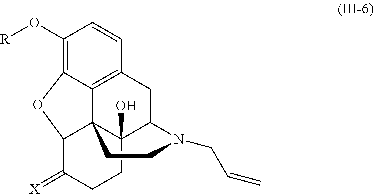

wherein:
X is O or CH₂;
R is —C(O)R¹;
wherein R¹ is

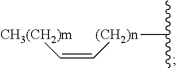

and
wherein m and n are integers between 0 and 23, and the sum of m and n is an integer between 6 and 23.

In one specific embodiment, m is 0, n is 23, and X is O.

In another specific embodiment, m is 0, n is 23, and X is CH₂.

In yet another specific embodiment, m is 1, n is 22, and X is O.

In yet another specific embodiment, m is 1, n is 22, and X is CH₂.

In yet another specific embodiment, m is 2, n is 21, and X is O.

In yet another specific embodiment, m is 2, n is 21, and X is CH₂.

In yet another specific embodiment, m is 3, n is 20, and X is O.

In yet another specific embodiment, m is 3, n is 20, and X is CH₂.

In yet another specific embodiment, m is 4, n is 19, and X is O.

In yet another specific embodiment, m is 4, n is 19, and X is CH₂.

In yet another specific embodiment, m is 5, n is 18, and X is O.

In yet another specific embodiment, m is 5, n is 18, and X is CH₂.

In yet another specific embodiment, m is 6, n is 17, and X is O.

In yet another specific embodiment, m is 6, n is 17, and X is CH₂.

In yet another specific embodiment, m is 7, n is 16, and X is O.

In yet another specific embodiment, m is 7, n is 16, and X is CH₂.

In yet another specific embodiment, m is 8, n is 15, and X is O.

In yet another specific embodiment, m is 8, n is 15, and X is CH₂.

In yet another specific embodiment, m is 9, n is 14, and X is O.

In yet another specific embodiment, m is 9, n is 14, and X is CH₂.

In yet another specific embodiment, m is 10, n is 13, and X is O.

In yet another specific embodiment, m is 10, n is 13, and X is CH₂.

In yet another specific embodiment, m is 11, n is 12, and X is O.

In yet another specific embodiment, m is 11, n is 12, and X is CH₂.

In yet another specific embodiment, m is 12, n is 11, and X is O.

In yet another specific embodiment, m is 12, n is 11, and X is CH₂.

In yet another specific embodiment, m is 13, n is 10, and X is O.

In yet another specific embodiment, m is 13, n is 10, and X is CH₂.

In yet another specific embodiment, m is 14, n is 9, and X is O.

In yet another specific embodiment, m is 14, n is 9, and X is CH₂.

In yet another specific embodiment, m is 15, n is 8, and X is O.

In yet another specific embodiment, m is 15, n is 8, and X is CH₂.

In yet another specific embodiment, m is 16, n is 7, and X is O.

In yet another specific embodiment, m is 16, n is 7, and X is CH₂.

In yet another specific embodiment, m is 17, n is 6, and X is O.

In yet another specific embodiment, m is 17, n is 6, and X is CH₂.

In yet another specific embodiment, m is 18, n is 5, and X is O.

In yet another specific embodiment, m is 18, n is 5, and X is CH₂.

In yet another specific embodiment, m is 19, n is 4, and X is O.

In yet another specific embodiment, m is 19, n is 4, and X is CH₂.

In yet another specific embodiment, m is 20, n is 3, and X is O.

In yet another specific embodiment, m is 20, n is 3, and X is CH₂.

In yet another specific embodiment, m is 21, n is 2, and X is O.

In yet another specific embodiment, m is 21, n is 2, and X is CH₂.

In yet another specific embodiment, m is 22, n is 1, and X is O.

In yet another specific embodiment, m is 22, n is 1, and X is CH₂.

In yet another specific embodiment, m is 23, n is 0, and X is O.

In yet another specific embodiment, m is 23, n is 0, and X is CH₂.

In another specific aspect, X, m and n in Formula (III-6) are as described in the First Specific Embodiment for Formula (I-5); and R and R¹ are as described above for Formula (III-6).

In another specific aspect, X, m and n in Formula (III-6) are as described in the Second Specific Embodiment for Formula (I-5); and R and R¹ are as described above for Formula (III-6).

In another specific aspect, X, m and n in Formula (III-6) are as described in the Third Specific Embodiment for Formula (I-5); and R and R¹ are as described above for Formula (III-6).

In another specific aspect, X, m and n in Formula (III-6) are as described in the Fourth Specific Embodiment for Formula (I-5); and R and R¹ are as described above for Formula (III-6).

In another specific aspect, X, m and n in Formula (III-6) are as described in the Fifth Specific Embodiment for Formula (I-5); and R and R¹ are as described above for Formula (III-6).

In another specific aspect, X, m and n in Formula (III-6) are as described in the Sixth Specific Embodiment for Formula (I-5); and R and R¹ are as described above for Formula (III-6).

Pharmaceutically acceptable salts of any one of the foregoing embodiments described in the 33^{rd} aspect are also included.

In a 34^{th} aspect, provided herein is a compound, or pharmaceutically acceptable salt thereof, having a structure provided in Formula (III-7),

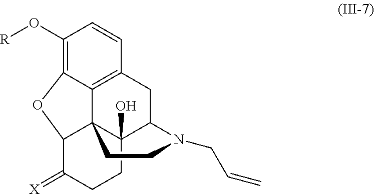

wherein:
X is O or CH₂;
R is —C(O)OR¹;
wherein R¹ is

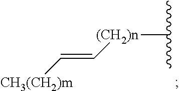

and
wherein m and n are integers between 0 and 23, and the sum of m and n is an integer between 6 and 23.

In one specific embodiment, m is 0, n is 23, and X is O.

In another specific embodiment, m is 0, n is 23, and X is CH₂.

In yet another specific embodiment, m is 1, n is 22, and X is O.

In yet another specific embodiment, m is 1, n is 22, and X is CH₂.

In yet another specific embodiment, m is 2, n is 21, and X is O.

In yet another specific embodiment, m is 2, n is 21, and X is CH₂.

In yet another specific embodiment, m is 3, n is 20, and X is O.

In yet another specific embodiment, m is 3, n is 20, and X is CH₂.

In yet another specific embodiment, m is 4, n is 19, and X is O.

In yet another specific embodiment, m is 4, n is 19, and X is CH₂.

In yet another specific embodiment, m is 5, n is 18, and X is O.

In yet another specific embodiment, m is 5, n is 18, and X is CH₂.

In yet another specific embodiment, m is 6, n is 17, and X is O.

In yet another specific embodiment, m is 6, n is 17, and X is CH₂.

In yet another specific embodiment, m is 7, n is 16, and X is O.

In yet another specific embodiment, m is 7, n is 16, and X is CH₂.

In yet another specific embodiment, m is 8, n is 15, and X is O.

In yet another specific embodiment, m is 8, n is 15, and X is CH₂.

In yet another specific embodiment, m is 9, n is 14, and X is O.

In yet another specific embodiment, m is 9, n is 14, and X is CH₂.

In yet another specific embodiment, m is 10, n is 13, and X is O.

In yet another specific embodiment, m is 10, n is 13, and X is CH₂.

In yet another specific embodiment, m is 11, n is 12, and X is O.

In yet another specific embodiment, m is 11, n is 12, and X is CH₂.

In yet another specific embodiment, m is 12, n is 11, and X is O.

In yet another specific embodiment, m is 12, n is 11, and X is CH₂.

In yet another specific embodiment, m is 13, n is 10, and X is O.

In yet another specific embodiment, m is 13, n is 10, and X is CH₂.

In yet another specific embodiment, m is 14, n is 9, and X is O.

In yet another specific embodiment, m is 14, n is 9, and X is CH₂.

In yet another specific embodiment, m is 15, n is 8, and X is O.

In yet another specific embodiment, m is 15, n is 8, and X is CH₂.

In yet another specific embodiment, m is 16, n is 7, and X is O.

In yet another specific embodiment, m is 16, n is 7, and X is CH₂.

In yet another specific embodiment, m is 17, n is 6, and X is O.

In yet another specific embodiment, m is 17, n is 6, and X is CH₂.

In yet another specific embodiment, m is 18, n is 5, and X is O.

In yet another specific embodiment, m is 18, n is 5, and X is CH₂.

In yet another specific embodiment, m is 19, n is 4, and X is O.

In yet another specific embodiment, m is 19, n is 4, and X is CH₂.

In yet another specific embodiment, m is 20, n is 3, and X is O.

In yet another specific embodiment, m is 20, n is 3, and X is CH₂.

In yet another specific embodiment, m is 21, n is 2, and X is O.

In yet another specific embodiment, m is 21, n is 2, and X is CH₂.

In yet another specific embodiment, m is 22, n is 1, and X is O.

In yet another specific embodiment, m is 22, n is 1, and X is CH₂.

In yet another specific embodiment, m is 23, n is 0, and X is O.

In yet another specific embodiment, m is 23, n is 0, and X is CH₂.

In another specific aspect, X, m and n in Formula (III-7) are as described in the First Specific Embodiment for Formula (I-5); and R and R¹ are as described above for Formula (III-7).

In another specific aspect, X, m and n in Formula (III-7) are as described in the Second Specific Embodiment for Formula (I-5); and R and R¹ are as described above for Formula (III-7).

In another specific aspect, X, m and n in Formula (III-7) are as described in the Third Specific Embodiment for Formula (I-5); and R and R¹ are as described above for Formula (III-7).

In another specific aspect, X, m and n in Formula (III-7) are as described in the Fourth Specific Embodiment for Formula (I-5); and R and R¹ are as described above for Formula (III-7).

In another specific aspect, X, m and n in Formula (III-7) are as described in the Fifth Specific Embodiment for Formula (I-5); and R and R¹ are as described above for Formula (III-7).

In another specific aspect, X, m and n in Formula (III-7) are as described in the Sixth Specific Embodiment for Formula (I-5); and R and R¹ are as described above for Formula (III-7).

Pharmaceutically acceptable salts of any one of the foregoing embodiments described in the 34^{th} aspect are also included.

In a 35^{th} aspect, provided herein is a compound, or pharmaceutically acceptable salt thereof, having a structure provided in Formula (III-8),

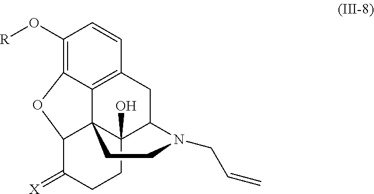

wherein:
X is O or CH₂;
R is —C(O)OR¹;
wherein R¹ is

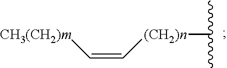

and
wherein m and n are integers between 0 and 23, and the sum of m and n is an integer between 6 and 23.

In one specific embodiment, m is 0, n is 23, and X is O.

In another specific embodiment, m is 0, n is 23, and X is CH₂.

In yet another specific embodiment, m is 1, n is 22, and X is O.

In yet another specific embodiment, m is 1, n is 22, and X is CH₂.

In yet another specific embodiment, m is 2, n is 21, and X is O.

In yet another specific embodiment, m is 2, n is 21, and X is CH₂.

In yet another specific embodiment, m is 3, n is 20, and X is O.

In yet another specific embodiment, m is 3, n is 20, and X is CH₂.

In yet another specific embodiment, m is 4, n is 19, and X is O.

In yet another specific embodiment, m is 4, n is 19, and X is CH₂.

In yet another specific embodiment, m is 5, n is 18, and X is O.

In yet another specific embodiment, m is 5, n is 18, and X is CH₂.

In yet another specific embodiment, m is 6, n is 17, and X is O.

In yet another specific embodiment, m is 6, n is 17, and X is CH₂.

In yet another specific embodiment, m is 7, n is 16, and X is O.

In yet another specific embodiment, m is 7, n is 16, and X is CH₂.

In yet another specific embodiment, m is 8, n is 15, and X is O.

In yet another specific embodiment, m is 8, n is 15, and X is CH₂.

In yet another specific embodiment, m is 9, n is 14, and X is O.

In yet another specific embodiment, m is 9, n is 14, and X is CH₂.

In yet another specific embodiment, m is 10, n is 13, and X is O.

In yet another specific embodiment, m is 10, n is 13, and X is CH₂.

In yet another specific embodiment, m is 11, n is 12, and X is O.

In yet another specific embodiment, m is 11, n is 12, and X is CH₂.

In yet another specific embodiment, m is 12, n is 11, and X is O.

In yet another specific embodiment, m is 12, n is 11, and X is CH₂.

In yet another specific embodiment, m is 13, n is 10, and X is O.

In yet another specific embodiment, m is 13, n is 10, and X is CH₂.

In yet another specific embodiment, m is 14, n is 9, and X is O.

In yet another specific embodiment, m is 14, n is 9, and X is CH₂.

In yet another specific embodiment, m is 15, n is 8, and X is O.

In yet another specific embodiment, m is 15, n is 8, and X is CH₂.

In yet another specific embodiment, m is 16, n is 7, and X is O.

In yet another specific embodiment, m is 16, n is 7, and X is CH₂.

In yet another specific embodiment, m is 17, n is 6, and X is O.

In yet another specific embodiment, m is 17, n is 6, and X is CH₂.

In yet another specific embodiment, m is 18, n is 5, and X is O.

In yet another specific embodiment, m is 18, n is 5, and X is CH₂.

In yet another specific embodiment, m is 19, n is 4, and X is O.

In yet another specific embodiment, m is 19, n is 4, and X is CH₂.

In yet another specific embodiment, m is 20, n is 3, and X is O.

In yet another specific embodiment, m is 20, n is 3, and X is CH₂.

In yet another specific embodiment, m is 21, n is 2, and X is O.

In yet another specific embodiment, m is 21, n is 2, and X is CH₂.

In yet another specific embodiment, m is 22, n is 1, and X is O.

In yet another specific embodiment, m is 22, n is 1, and X is CH₂.

In yet another specific embodiment, m is 23, n is 0, and X is O.

In yet another specific embodiment, m is 23, n is 0, and X is CH₂.

In another specific aspect, X, m and n in Formula (III-8) are as described in the First Specific Embodiment for Formula (I-5); and R and R¹ are as described above for Formula (III-8).

In another specific aspect, X, m and n in Formula (III-8) are as described in the Second Specific Embodiment for Formula (I-5); and R and R¹ are as described above for Formula (III-8).

In another specific aspect, X, m and n in Formula (III-8) are as described in the Third Specific Embodiment for Formula (I-5); and R and R¹ are as described above for Formula (III-8).

In another specific aspect, X, m and n in Formula (III-8) are as described in the Fourth Specific Embodiment for Formula (I-5); and R and R¹ are as described above for Formula (III-8).

In another specific aspect, X, m and n in Formula (III-8) are as described in the Fifth Specific Embodiment for Formula (I-5); and R and R¹ are as described above for Formula (III-8).

In another specific aspect, X, m and n in Formula (III-8) are as described in the Sixth Specific Embodiment for Formula (I-5); and R and R¹ are as described above for Formula (III-8).

Pharmaceutically acceptable salts of any one of the foregoing embodiments described in the 35^{th} aspect are also included.

In a 36^{th} aspect, provided herein is a compound, or pharmaceutically acceptable salt thereof, having a structure provided in Formula (III-9),

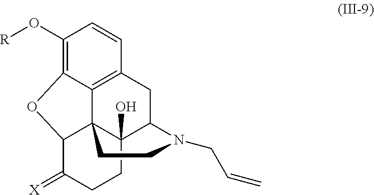

wherein:
X is O or CH₂;
R is —CH₂OC(O)R¹;
wherein R¹ is

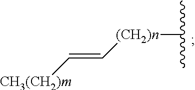

and
wherein m and n are integers between 0 and 23, and the sum of m and n is an integer between 6 and 23.

In one specific embodiment, m is 0, n is 23, and X is O.

In another specific embodiment, m is 0, n is 23, and X is CH₂.

In yet another specific embodiment, m is 1, n is 22, and X is O.

In yet another specific embodiment, m is 1, n is 22, and X is CH₂.

In yet another specific embodiment, m is 2, n is 21, and X is O.

In yet another specific embodiment, m is 2, n is 21, and X is CH₂.

In yet another specific embodiment, m is 3, n is 20, and X is O.

In yet another specific embodiment, m is 3, n is 20, and X is CH₂.

In yet another specific embodiment, m is 4, n is 19, and X is O.

In yet another specific embodiment, m is 4, n is 19, and X is CH₂.

In yet another specific embodiment, m is 5, n is 18, and X is O.

In yet another specific embodiment, m is 5, n is 18, and X is CH₂.

In yet another specific embodiment, m is 6, n is 17, and X is O.

In yet another specific embodiment, m is 6, n is 17, and X is CH₂.

In yet another specific embodiment, m is 7, n is 16, and X is O.

In yet another specific embodiment, m is 7, n is 16, and X is CH₂.

In yet another specific embodiment, m is 8, n is 15, and X is O.

In yet another specific embodiment, m is 8, n is 15, and X is CH₂.

In yet another specific embodiment, m is 9, n is 14, and X is O.

In yet another specific embodiment, m is 9, n is 14, and X is CH₂.

In yet another specific embodiment, m is 10, n is 13, and X is O.

In yet another specific embodiment, m is 10, n is 13, and X is CH₂.

In yet another specific embodiment, m is 11, n is 12, and X is O.

In yet another specific embodiment, m is 11, n is 12, and X is CH₂.

In yet another specific embodiment, m is 12, n is 11, and X is O.

In yet another specific embodiment, m is 12, n is 11, and X is CH₂.

In yet another specific embodiment, m is 13, n is 10, and X is O.

In yet another specific embodiment, m is 13, n is 10, and X is CH₂.

In yet another specific embodiment, m is 14, n is 9, and X is O.

In yet another specific embodiment, m is 14, n is 9, and X is CH₂.

In yet another specific embodiment, m is 15, n is 8, and X is O.

In yet another specific embodiment, m is 15, n is 8, and X is CH₂.

In yet another specific embodiment, m is 16, n is 7, and X is O.

In yet another specific embodiment, m is 16, n is 7, and X is CH₂.

In yet another specific embodiment, m is 17, n is 6, and X is O.

In yet another specific embodiment, m is 17, n is 6, and X is CH₂.

In yet another specific embodiment, m is 18, n is 5, and X is O.

In yet another specific embodiment, m is 18, n is 5, and X is CH₂.

In yet another specific embodiment, m is 19, n is 4, and X is O.

In yet another specific embodiment, m is 19, n is 4, and X is CH₂.

In yet another specific embodiment, m is 20, n is 3, and X is O.

In yet another specific embodiment, m is 20, n is 3, and X is CH₂.

In yet another specific embodiment, m is 21, n is 2, and X is O.

In yet another specific embodiment, m is 21, n is 2, and X is CH₂.

In yet another specific embodiment, m is 22, n is 1, and X is O.

In yet another specific embodiment, m is 22, n is 1, and X is CH₂.

In yet another specific embodiment, m is 23, n is 0, and X is O.

In yet another specific embodiment, m is 23, n is 0, and X is CH₂.

In another specific aspect, X, m and n in Formula (III-9) are as described in the First Specific Embodiment for Formula (I-5); and R and R¹ are as described above for Formula (III-9).

In another specific aspect, X, m and n in Formula (III-9) are as described in the Second Specific Embodiment for Formula (I-5); and R and R¹ are as described above for Formula (III-9).

In another specific aspect, X, m and n in Formula (III-9) are as described in the Third Specific Embodiment for Formula (I-5); and R and R¹ are as described above for Formula (III-9).

In another specific aspect, X, m and n in Formula (III-9) are as described in the Fourth Specific Embodiment for Formula (I-5); and R and R¹ are as described above for Formula (III-9).

In another specific aspect, X, m and n in Formula (III-9) are as described in the Fifth Specific Embodiment for Formula (I-5); and R and R¹ are as described above for Formula (III-9).

In another specific aspect, X, m and n in Formula (III-9) are as described in the Sixth Specific Embodiment for Formula (I-5); and R and R¹ are as described above for Formula (III-9).

Pharmaceutically acceptable salts of any one of the foregoing embodiments described in the 36^{th} aspect are also included.

In a 37^{th} aspect, provided herein is a compound, or pharmaceutically acceptable salt thereof, having a structure provided in Formula (III-10),

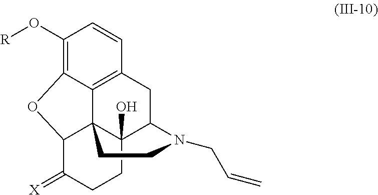

wherein:
X is O or CH₂;
R is —CH₂OC(O)R¹;
wherein R¹ is

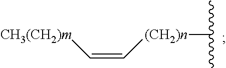

and
wherein m and n are integers between 0 and 23, and the sum of m and n is an integer between 6 and 23.

In one specific embodiment, m is 0, n is 23, and X is O.

In another specific embodiment, m is 0, n is 23, and X is CH₂.

In yet another specific embodiment, m is 1, n is 22, and X is O.

In yet another specific embodiment, m is 1, n is 22, and X is CH₂.

In yet another specific embodiment, m is 2, n is 21, and X is O.

In yet another specific embodiment, m is 2, n is 21, and X is CH₂.

In yet another specific embodiment, m is 3, n is 20, and X is O.

In yet another specific embodiment, m is 3, n is 20, and X is CH₂.

In yet another specific embodiment, m is 4, n is 19, and X is O.

In yet another specific embodiment, m is 4, n is 19, and X is CH₂.

In yet another specific embodiment, m is 5, n is 18, and X is O.

In yet another specific embodiment, m is 5, n is 18, and X is CH₂.

In yet another specific embodiment, m is 6, n is 17, and X is O.

In yet another specific embodiment, m is 6, n is 17, and X is CH₂.

In yet another specific embodiment, m is 7, n is 16, and X is O.

In yet another specific embodiment, m is 7, n is 16, and X is CH₂.

In yet another specific embodiment, m is 8, n is 15, and X is O.

In yet another specific embodiment, m is 8, n is 15, and X is CH₂.

In yet another specific embodiment, m is 9, n is 14, and X is O.

In yet another specific embodiment, m is 9, n is 14, and X is CH₂.

In yet another specific embodiment, m is 10, n is 13, and X is O.

In yet another specific embodiment, m is 10, n is 13, and X is CH₂.

In yet another specific embodiment, m is 11, n is 12, and X is O.

In yet another specific embodiment, m is 11, n is 12, and X is CH₂.

In yet another specific embodiment, m is 12, n is 11, and X is O.

In yet another specific embodiment, m is 12, n is 11, and X is CH₂.

In yet another specific embodiment, m is 13, n is 10, and X is O.

In yet another specific embodiment, m is 13, n is 10, and X is CH₂.

In yet another specific embodiment, m is 14, n is 9, and X is O.

In yet another specific embodiment, m is 14, n is 9, and X is CH₂.

In yet another specific embodiment, m is 15, n is 8, and X is O.

In yet another specific embodiment, m is 15, n is 8, and X is CH₂.

In yet another specific embodiment, m is 16, n is 7, and X is O.

In yet another specific embodiment, m is 16, n is 7, and X is CH₂.

In yet another specific embodiment, m is 17, n is 6, and X is O.

In yet another specific embodiment, m is 17, n is 6, and X is CH₂.

In yet another specific embodiment, m is 18, n is 5, and X is O.

In yet another specific embodiment, m is 18, n is 5, and X is CH₂.

In yet another specific embodiment, m is 19, n is 4, and X is O.

In yet another specific embodiment, m is 19, n is 4, and X is CH₂.

In yet another specific embodiment, m is 20, n is 3, and X is O.

In yet another specific embodiment, m is 20, n is 3, and X is CH₂.

In yet another specific embodiment, m is 21, n is 2, and X is O.

In yet another specific embodiment, m is 21, n is 2, and X is CH₂.

In yet another specific embodiment, m is 22, n is 1, and X is O.

In yet another specific embodiment, m is 22, n is 1, and X is CH₂.

In yet another specific embodiment, m is 23, n is 0, and X is O.

In yet another specific embodiment, m is 23, n is 0, and X is CH₂.

In another specific aspect, X, m and n in Formula (III-10) are as described in the First Specific Embodiment for Formula (I-5); and R and R¹ are as described above for Formula (III-10).

In another specific aspect, X, m and n in Formula (III-10) are as described in the Second Specific Embodiment for Formula (I-5); and R and R¹ are as described above for Formula (III-10).

In another specific aspect, X, m and n in Formula (III-10) are as described in the Third Specific Embodiment for Formula (I-5); and R and R¹ are as described above for Formula (III-10).

In another specific aspect, X, m and n in Formula (III-10) are as described in the Fourth Specific Embodiment for Formula (I-5); and R and R¹ are as described above for Formula (III-10).

In another specific aspect, X, m and n in Formula (III-10) are as described in the Fifth Specific Embodiment for Formula (I-5); and R and R¹ are as described above for Formula (III-10).

In another specific aspect, X, m and n in Formula (III-10) are as described in the Sixth Specific Embodiment for Formula (I-5); and R and R¹ are as described above for Formula (III-10).

Pharmaceutically acceptable salts of any one of the foregoing embodiments described in the 37^{th} aspect are also included.

In a 38^{th} aspect, provided herein is a compound, or pharmaceutically acceptable salt thereof, having a structure provided in Formula (III-11),

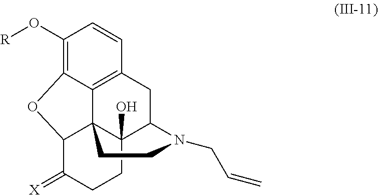

wherein:
X is O or CH₂;
R is —CH₂OC(O)OR¹;
wherein R¹ is

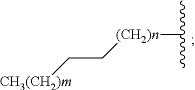

and
wherein m and n are integers between 0 and 23, and the sum of m and n is an integer between 6 and 23.

In one specific embodiment, m is 0, n is 23, and X is O.

In another specific embodiment, m is 0, n is 23, and X is CH₂.

In yet another specific embodiment, m is 1, n is 22, and X is O.

In yet another specific embodiment, m is 1, n is 22, and X is CH₂.

In yet another specific embodiment, m is 2, n is 21, and X is O.

In yet another specific embodiment, m is 2, n is 21, and X is CH₂.

In yet another specific embodiment, m is 3, n is 20, and X is O.

In yet another specific embodiment, m is 3, n is 20, and X is CH₂.

In yet another specific embodiment, m is 4, n is 19, and X is O.

In yet another specific embodiment, m is 4, n is 19, and X is CH₂.

In yet another specific embodiment, m is 5, n is 18, and X is O.

In yet another specific embodiment, m is 5, n is 18, and X is CH₂.

In yet another specific embodiment, m is 6, n is 17, and X is O.

In yet another specific embodiment, m is 6, n is 17, and X is CH₂.

In yet another specific embodiment, m is 7, n is 16, and X is O.

In yet another specific embodiment, m is 7, n is 16, and X is CH₂.

In yet another specific embodiment, m is 8, n is 15, and X is O.

In yet another specific embodiment, m is 8, n is 15, and X is CH₂.

In yet another specific embodiment, m is 9, n is 14, and X is O.

In yet another specific embodiment, m is 9, n is 14, and X is CH₂.

In yet another specific embodiment, m is 10, n is 13, and X is O.

In yet another specific embodiment, m is 10, n is 13, and X is CH₂.

In yet another specific embodiment, m is 11, n is 12, and X is O.

In yet another specific embodiment, m is 11, n is 12, and X is CH₂.

In yet another specific embodiment, m is 12, n is 11, and X is O.

In yet another specific embodiment, m is 12, n is 11, and X is CH₂.

In yet another specific embodiment, m is 13, n is 10, and X is O.

In yet another specific embodiment, m is 13, n is 10, and X is CH₂.

In yet another specific embodiment, m is 14, n is 9, and X is O.

In yet another specific embodiment, m is 14, n is 9, and X is CH₂.

In yet another specific embodiment, m is 15, n is 8, and X is O.

In yet another specific embodiment, m is 15, n is 8, and X is CH₂.

In yet another specific embodiment, m is 16, n is 7, and X is O.

In yet another specific embodiment, m is 16, n is 7, and X is CH₂.

In yet another specific embodiment, m is 17, n is 6, and X is O.

In yet another specific embodiment, m is 17, n is 6, and X is CH₂.

In yet another specific embodiment, m is 18, n is 5, and X is O.

In yet another specific embodiment, m is 18, n is 5, and X is CH₂.

In yet another specific embodiment, m is 19, n is 4, and X is O.

In yet another specific embodiment, m is 19, n is 4, and X is CH₂.

In yet another specific embodiment, m is 20, n is 3, and X is O.

In yet another specific embodiment, m is 20, n is 3, and X is CH₂.

In yet another specific embodiment, m is 21, n is 2, and X is O.

In yet another specific embodiment, m is 21, n is 2, and X is CH₂.

In yet another specific embodiment, m is 22, n is 1, and X is O.

In yet another specific embodiment, m is 22, n is 1, and X is CH₂.

In yet another specific embodiment, m is 23, n is 0, and X is O.

In yet another specific embodiment, m is 23, n is 0, and X is CH₂.

In another specific aspect, X, m and n in Formula (III-11) are as described in the First Specific Embodiment for Formula (I-5); and R and R¹ are as described above for Formula (III-11).

In another specific aspect, X, m and n in Formula (III-11) are as described in the Second Specific Embodiment for Formula (I-5); and R and R¹ are as described above for Formula (III-11).

In another specific aspect, X, m and n in Formula (III-11) are as described in the Third Specific Embodiment for Formula (I-5); and R and R¹ are as described above for Formula (III-11).

In another specific aspect, X, m and n in Formula (III-11) are as described in the Fourth Specific Embodiment for Formula (I-5); and R and R¹ are as described above for Formula (III-11).

In another specific aspect, X, m and n in Formula (III-11) are as described in the Fifth Specific Embodiment for Formula (I-5); and R and R¹ are as described above for Formula (III-11).

In another specific aspect, X, m and n in Formula (III-11) are as described in the Sixth Specific Embodiment for Formula (I-5); and R and R¹ are as described above for Formula (III-11).

Pharmaceutically acceptable salts of any one of the foregoing embodiments described in the 38^{th} aspect are also included.

In a 39^{th} aspect, provided herein is a compound, or pharmaceutically acceptable salt thereof, having a structure provided in Formula (III-12),

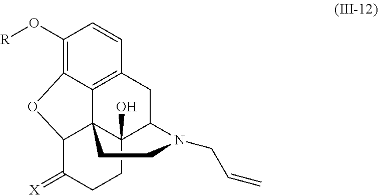

wherein:
X is O or CH₂;
R is —CH₂OC(O)OR¹;
wherein R¹ is

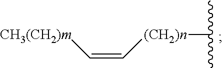

and
wherein m and n are integers between 0 and 23, and the sum of m and n is an integer between 6 and 23.

In one specific embodiment, m is 0, n is 23, and X is O.

In another specific embodiment, m is 0, n is 23, and X is CH₂.

In yet another specific embodiment, m is 1, n is 22, and X is O.

In yet another specific embodiment, m is 1, n is 22, and X is CH₂.

In yet another specific embodiment, m is 2, n is 21, and X is O.

In yet another specific embodiment, m is 2, n is 21, and X is CH₂.

In yet another specific embodiment, m is 3, n is 20, and X is O.

In yet another specific embodiment, m is 3, n is 20, and X is CH₂.

In yet another specific embodiment, m is 4, n is 19, and X is O.

In yet another specific embodiment, m is 4, n is 19, and X is CH₂.

In yet another specific embodiment, m is 5, n is 18, and X is O.

In yet another specific embodiment, m is 5, n is 18, and X is CH₂.

In yet another specific embodiment, m is 6, n is 17, and X is O.

In yet another specific embodiment, m is 6, n is 17, and X is CH₂.

In yet another specific embodiment, m is 7, n is 16, and X is O.

In yet another specific embodiment, m is 7, n is 16, and X is CH₂.

In yet another specific embodiment, m is 8, n is 15, and X is O.

In yet another specific embodiment, m is 8, n is 15, and X is CH₂.

In yet another specific embodiment, m is 9, n is 14, and X is O.

In yet another specific embodiment, m is 9, n is 14, and X is CH₂.

In yet another specific embodiment, m is 10, n is 13, and X is O.

In yet another specific embodiment, m is 10, n is 13, and X is CH₂.

In yet another specific embodiment, m is 11, n is 12, and X is O.

In yet another specific embodiment, m is 11, n is 12, and X is CH₂.

In yet another specific embodiment, m is 12, n is 11, and X is O.

In yet another specific embodiment, m is 12, n is 11, and X is CH₂.

In yet another specific embodiment, m is 13, n is 10, and X is O.

In yet another specific embodiment, m is 13, n is 10, and X is CH₂.

In yet another specific embodiment, m is 14, n is 9, and X is O.

In yet another specific embodiment, m is 14, n is 9, and X is CH₂.

In yet another specific embodiment, m is 15, n is 8, and X is O.

In yet another specific embodiment, m is 15, n is 8, and X is CH₂.

In yet another specific embodiment, m is 16, n is 7, and X is O.

In yet another specific embodiment, m is 16, n is 7, and X is CH₂.

In yet another specific embodiment, m is 17, n is 6, and X is O.

In yet another specific embodiment, m is 17, n is 6, and X is CH₂.

In yet another specific embodiment, m is 18, n is 5, and X is O.

In yet another specific embodiment, m is 18, n is 5, and X is CH₂.

In yet another specific embodiment, m is 19, n is 4, and X is O.

In yet another specific embodiment, m is 19, n is 4, and X is CH₂.

In yet another specific embodiment, m is 20, n is 3, and X is O.

In yet another specific embodiment, m is 20, n is 3, and X is CH₂.

In yet another specific embodiment, m is 21, n is 2, and X is O.

In yet another specific embodiment, m is 21, n is 2, and X is CH₂.

In yet another specific embodiment, m is 22, n is 1, and X is O.

In yet another specific embodiment, m is 22, n is 1, and X is CH₂.

In yet another specific embodiment, m is 23, n is 0, and X is O.

In yet another specific embodiment, m is 23, n is 0, and X is CH₂.

In another specific aspect, X, m and n in Formula (III-12) are as described in the First Specific Embodiment for Formula (I-5); and R and R¹ are as described above for Formula (III-12).

In another specific aspect, X, m and n in Formula (II-12) are as described in the Second Specific Embodiment for Formula (I-5); and R and R¹ are as described above for Formula (III-12).

In another specific aspect, X, m and n in Formula (III-12) are as described in the Third Specific Embodiment for Formula (I-5); and R and R¹ are as described above for Formula (III-12).

In another specific aspect, X, m and n in Formula (III-12) are as described in the Fourth Specific Embodiment for Formula (I-5); and R and R¹ are as described above for Formula (III-12).

In another specific aspect, X, m and n in Formula (III-12) are as described in the Fifth Specific Embodiment for Formula (I-5); and R and R¹ are as described above for Formula (III-12).

In another specific aspect, X, m and n in Formula (III-12) are as described in the Sixth Specific Embodiment for Formula (I-5); and R and R¹ are as described above for Formula (III-12).

Pharmaceutically acceptable salts of any one of the foregoing embodiments described in the 39^{th} aspect are also included.

In a 40^{th} aspect, provided herein is a compound, or pharmaceutically acceptable salt thereof, having a structure provided in Formula (I-13),

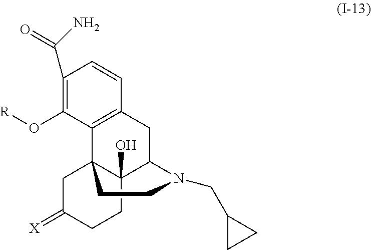

wherein X is ═O and R is any of the groups recited in Table 1, 2, 3 or 4.

In a 41^{st} aspect, provided herein is a compound, or pharmaceutically acceptable salt thereof, having a structure provided in Formula (I-14),

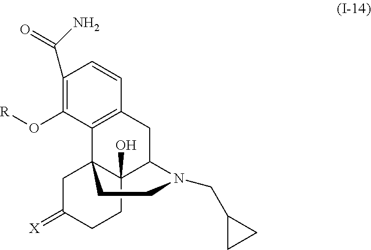

wherein X is ═CH₂ and R is any of the groups recited in Table 1, 2, 3 or 4.

In a 42^{nd} aspect, provided herein is a compound, or pharmaceutically acceptable salt thereof, having a structure provided in Formula (II-13),

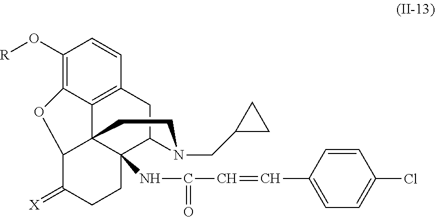

wherein X is ═O and R is any of the groups recited in Table 1, 2, 3 or 4.

In a 43^{rd} aspect, provided herein is a compound, or pharmaceutically acceptable salt thereof, having a structure provided in Formula (II-14),

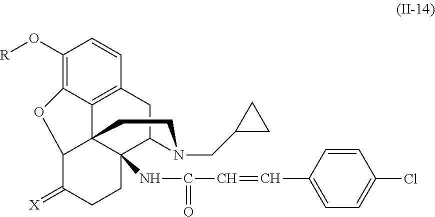

wherein X is ═CH₂ and R is any of the groups recited in Table 1, 2, 3 or 4.

In a 44^{th} aspect, provided herein is a compound, or pharmaceutically acceptable salt thereof, having a structure provided in Formula (III-13),

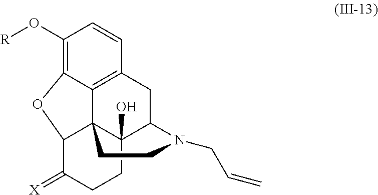

wherein X is ═O and R is any of the groups recited in Table 1, 2, 3 or 4.

In a 45^{th} aspect, provided herein is a compound, or pharmaceutically acceptable salt thereof, having a structure provided in Formula (III-14),

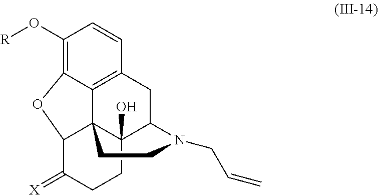

wherein X is ═CH₂ and R is any of the groups recited in Table 1, 2, 3 or 4.

TABLES 1-4

| | |
|---|---|
| 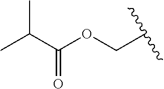 [SMILES: *COC(=O)C(C)C] | 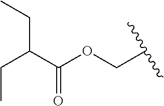 [SMILES: *COC(=O)C(CC)CC] |
| 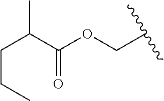 [SMILES: *COC(=O)C(C)CCC] | 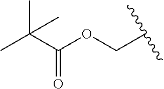 [SMILES: *COC(=O)C(C)(C)C] |
| 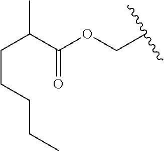 [SMILES: *COC(=O)C(C)CCCCC] | 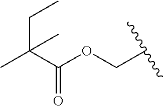 [SMILES: *COC(=O)C(C)(C)CC] |
| 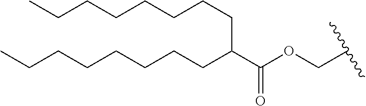 [SMILES: *COC(=O)C(CCCCCCCC)CCCCCCCC] | 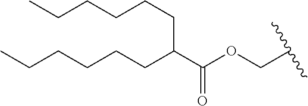 [SMILES: *COC(=O)C(CCCCCC)CCCCCC] |
| 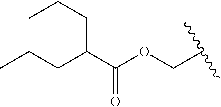 [SMILES: *COC(=O)C(CCC)CCC] | 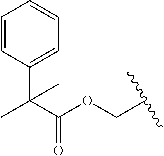 [SMILES: *COC(=O)C(C)(C)c1ccccc1] |
| 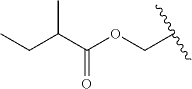 [SMILES: *COC(=O)C(C)CC] | 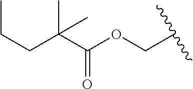 [SMILES: *COC(=O)C(C)(C)CCC] |
| 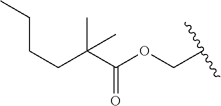 [SMILES: *COC(=O)C(C)(C)CCCC] | 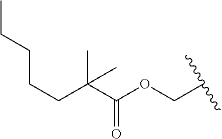 [SMILES: *COC(=O)C(C)(C)CCCCC] |
| 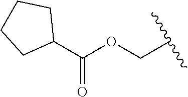 [SMILES: *COC(=O)C1CCCC1] | 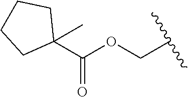 [SMILES: *COC(=O)C1(C)CCCC1] |
| 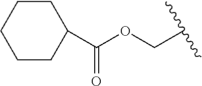 [SMILES: *COC(=O)C1CCCCC1] | 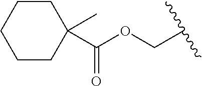 [SMILES: *COC(=O)C1(C)CCCCC1] |
| 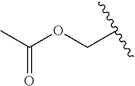 [SMILES: *COC(C)=O] | 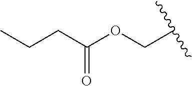 [SMILES: *COC(=O)CCC] |
| 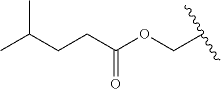 [SMILES: *COC(=O)CCC(C)C] | 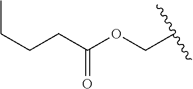 [SMILES: *COC(=O)CCCC] |
| 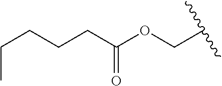 [SMILES: *COC(=O)CCCCC] | 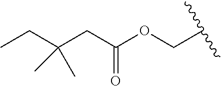 [SMILES: *COC(=O)CC(C)(C)CC] |
| 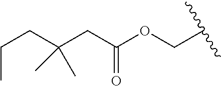 [SMILES: *COC(=O)CC(C)(C)CCC] | 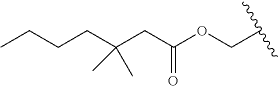 [SMILES: *COC(=O)CC(C)(C)CCCC] |
| 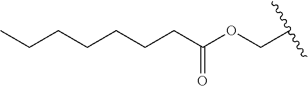 [SMILES: *COC(=O)CCCCCCC] | 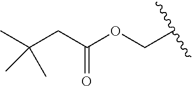 [SMILES: *COC(=O)CC(C)(C)C] |
| 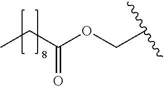 | 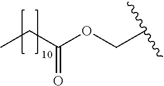 |
| 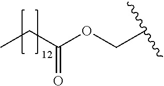 | 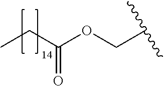 |
| 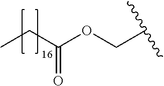 | 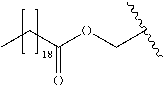 |
| 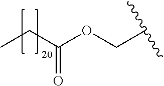 | 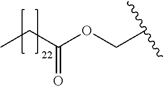 |
| 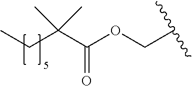 | 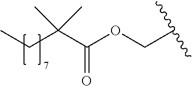 |
| 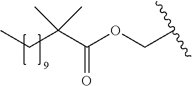 | 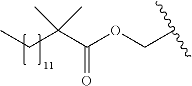 |
| 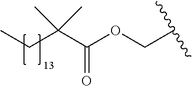 | 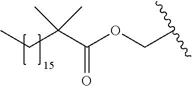 |
| 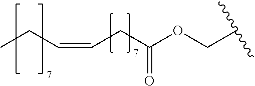 | 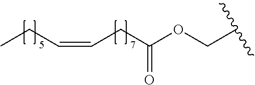 |
| 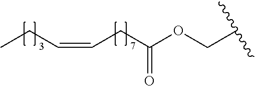 | 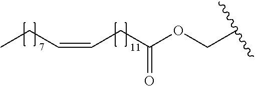 |
| 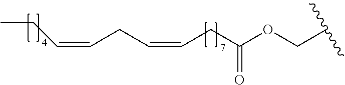 | 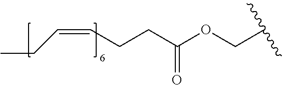 |
| 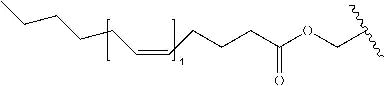 | 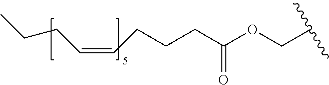 |
| 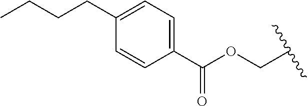 [SMILES: *COC(=O)c1ccc(CCCC)cc1] | 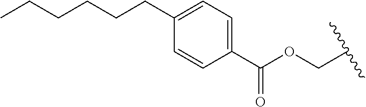 [SMILES: *COC(=O)c1ccc(CCCCCC)cc1] |
| 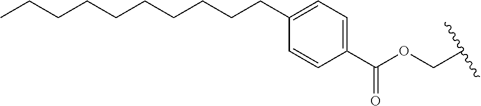 [SMILES: *COC(=O)c1ccc(CCCCCCCCCC)cc1] | 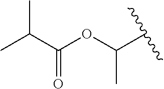 [SMILES: *C(C)OC(=O)C(C)C] |
| 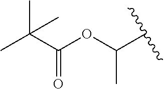 [SMILES: *C(C)OC(=O)C(C)(C)C] | 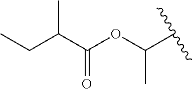 [SMILES: *C(C)OC(=O)C(C)CC] |
| 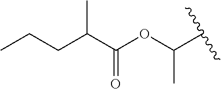 [SMILES: *C(C)OC(=O)C(C)CCC] | 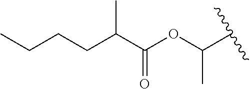 [SMILES: *C(C)OC(=O)C(C)CCCC] |
| 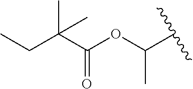 [SMILES: *C(C)OC(=O)C(C)(C)CC] | 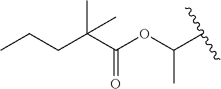 [SMILES: *C(C)OC(=O)C(C)(C)CCC] |
| 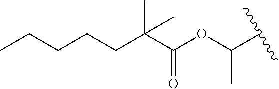 [SMILES: *C(C)OC(=O)C(C)(C)CCCCC] | 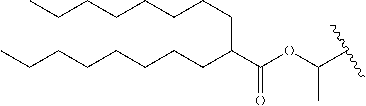 [SMILES: *C(C)OC(=O)C(CCCCCCCC)CCCCCCCC] |
| 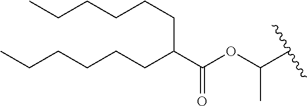 [SMILES: *C(C)OC(=O)C(CCCCCC)CCCCCC] | 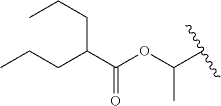 [SMILES: *C(C)OC(=O)C(CCC)CCC] |
| 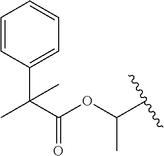 [SMILES: *C(C)OC(=O)C(C)(C)c1ccccc1] | 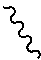 |
| 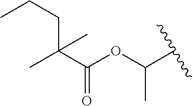 [SMILES: *C(C)OC(=O)C(C)(C)CCC] | 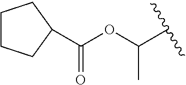 [SMILES: *C(C)OC(=O)C1CCCC1] |
| 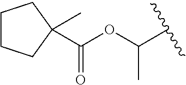 [SMILES: *C(C)OC(=O)C1(C)CCCC1] | 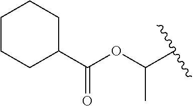 [SMILES: *C(C)OC(=O)C1CCCCC1] |
| 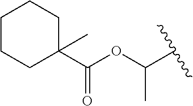 [SMILES: *C(C)OC(=O)C1(C)CCCCC1] | 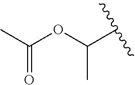 [SMILES: *C(C)OC(C)=O] |
| 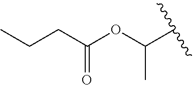 [SMILES: *C(C)OC(=O)CCC] | 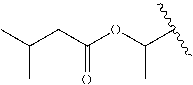 [SMILES: *C(C)OC(=O)CC(C)C] |
| 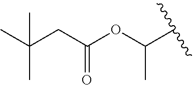 [SMILES: *C(C)OC(=O)CC(C)(C)C] | 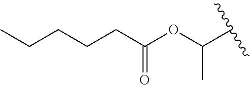 [SMILES: *C(C)OC(=O)CCCCC] |
| 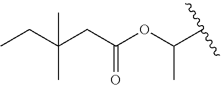 [SMILES: *C(C)OC(=O)CC(C)(C)CC] | 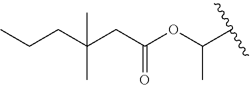 [SMILES: *C(C)OC(=O)CC(C)(C)CCC] |
| 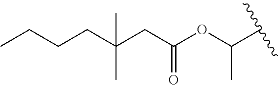 [SMILES: *C(C)OC(=O)CC(C)(C)CCCC] | 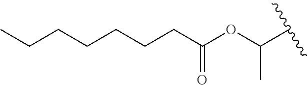 [SMILES: *C(C)OC(=O)CCCCCCC] |
| 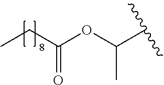 | 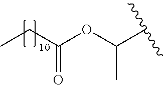 |
| 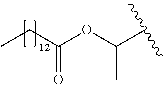 | 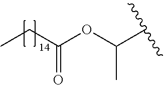 |
| 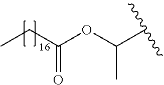 | 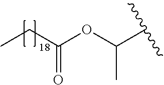 |
| 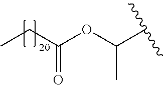 | 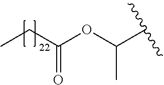 |
| 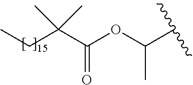 [SMILES: *C(C)OC(=O)C(C)(C)CC] | 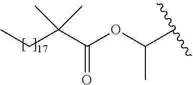 [SMILES: *C(C)OC(=O)C(C)(C)CC] |
| 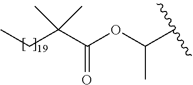 [SMILES: *C(C)OC(=O)C(C)(C)CC] | 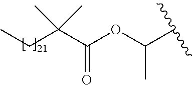 [SMILES: *C(C)OC(=O)C(C)(C)CC] |
| 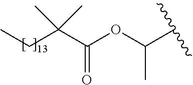 [SMILES: *C(C)OC(=O)C(C)(C)CC] | 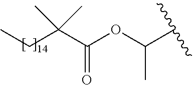 [SMILES: *C(C)OC(=O)C(C)(C)CC] |
| 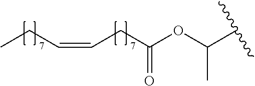 | 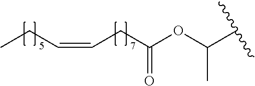 |
| 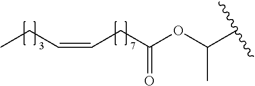 | 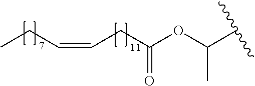 |
| 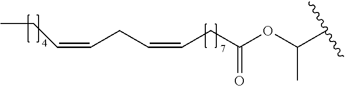 | 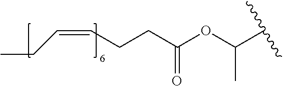 |
| 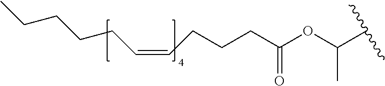 | 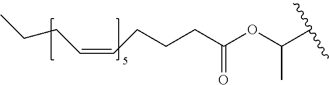 |

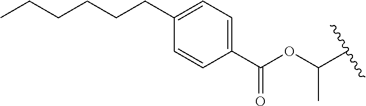 [SMILES: *C(C)OC(=O)c1ccc(CCCCCC)cc1]

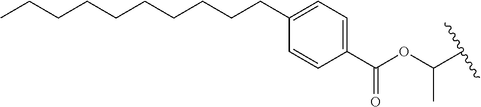 [SMILES: *C(C)OC(=O)c1ccc(CCCCCCCCCC)cc1]

| | |
|---|---|
| 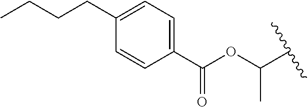 [SMILES: *C(C)OC(=O)c1ccc(CCCC)cc1] | 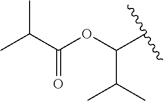 [SMILES: *C(OC(=O)C(C)C)C(C)C] |
| 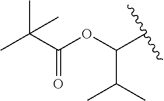 [SMILES: *C(OC(=O)C(C)(C)C)C(C)C] | 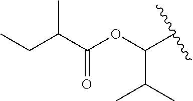 [SMILES: *C(OC(=O)C(C)CC)C(C)C] |
| 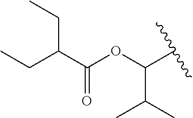 [SMILES: *C(OC(=O)C(CC)CC)C(C)C] | 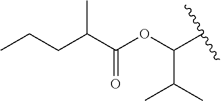 [SMILES: *C(OC(=O)C(C)CCC)C(C)C] |
| 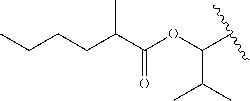 [SMILES: *C(OC(=O)C(C)CCCC)C(C)C] | 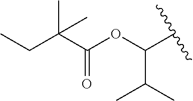 [SMILES: *C(OC(=O)C(C)(C)CC)C(C)C] |
| 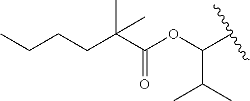 [SMILES: *C(OC(=O)C(C)(C)CCCC)C(C)C] | 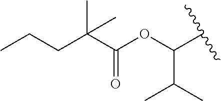 [SMILES: *C(OC(=O)C(C)(C)CCC)C(C)C] |
| 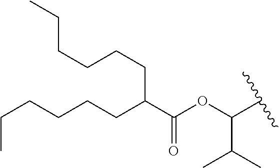 [SMILES: *C(OC(=O)C(CCCCCC)CCCCCC)C(C)C] | 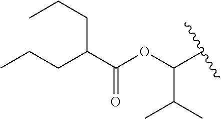 [SMILES: *C(OC(=O)C(CCC)CCC)C(C)C] |
| 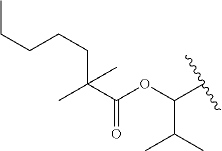 [SMILES: *C(OC(=O)C(C)(C)CCCCC)C(C)C] | 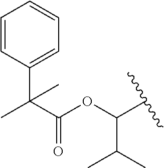 [SMILES: *C(OC(=O)C(C)(C)c1ccccc1)C(C)C] |
| 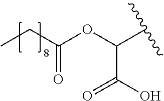 | 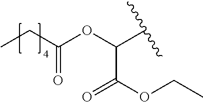 |
| 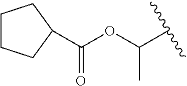 [SMILES: *C(C)OC(=O)C1CCCC1] | 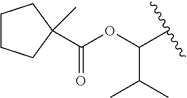 [SMILES: *C(OC(=O)C1(C)CCCC1)C(C)C] |
| 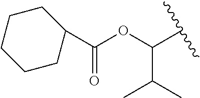 [SMILES: *C(OC(=O)C1CCCCC1)C(C)C] | 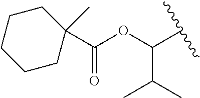 [SMILES: *C(OC(=O)C1(C)CCCCC1)C(C)C] |
| 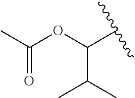 [SMILES: *C(OC(C)=O)C(C)C] | 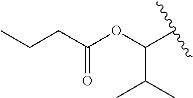 [SMILES: *C(OC(=O)CCC)C(C)C] |
| 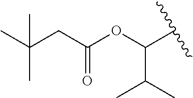 [SMILES: *C(OC(=O)CC(C)(C)C)C(C)C] | 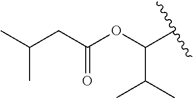 [SMILES: *C(OC(=O)CC(C)C)C(C)C] |
| 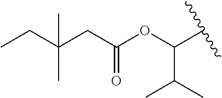 [SMILES: *C(OC(=O)CC(C)(C)CC)C(C)C] | 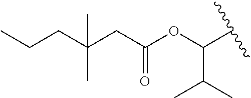 [SMILES: *C(OC(=O)CC(C)(C)CCC)C(C)C] |
| 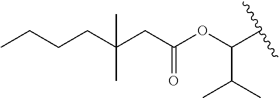 [SMILES: *C(OC(=O)CC(C)(C)CCCC)C(C)C] | 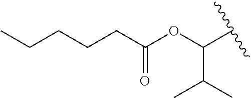 [SMILES: *C(OC(=O)CCCCC)C(C)C] |
| 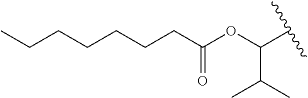 [SMILES: *C(OC(=O)CCCCCCC)C(C)C] | 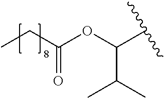 |
| 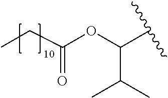 | 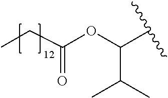 |
| 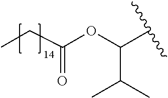 | 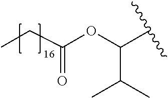 |
| 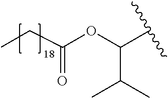 | 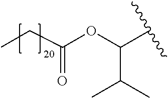 |
| 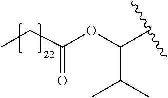 | 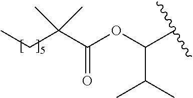 |
| 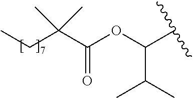 | 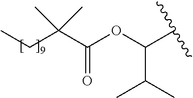 |
| 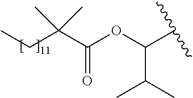 | 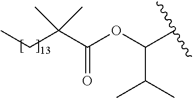 |
| 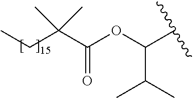 | 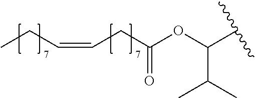 |
| 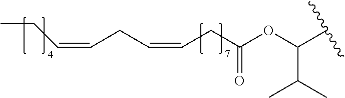 | 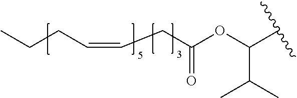 |
| 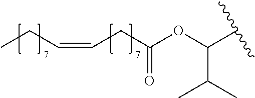 | 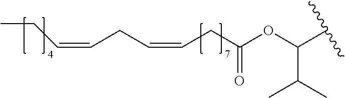 |
| 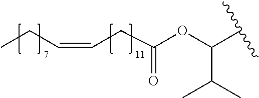 | 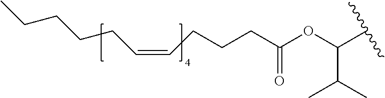 |

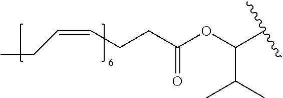

| | |
|---|---|
| 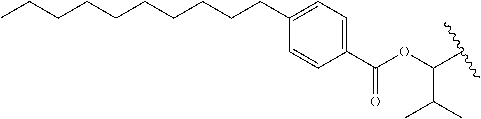 [SMILES: *C(OC(=O)c1ccc(CCCCCCCCCC)cc1)C(C)C] | 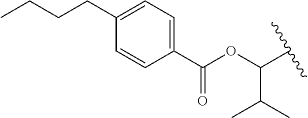 [SMILES: *C(OC(=O)c1ccc(CCCC)cc1)C(C)C] |
| 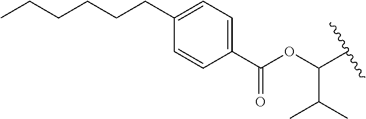 [SMILES: *C(OC(=O)c1ccc(CCCCCC)cc1)C(C)C] | 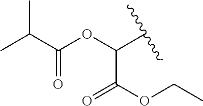 [SMILES: *C(OC(=O)C(C)C)C(=O)OCC] |
| 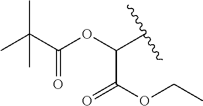 [SMILES: *C(OC(=O)C(C)(C)C)C(=O)OCC] | 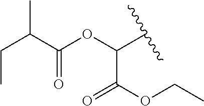 [SMILES: *C(OC(=O)C(C)CC)C(=O)OCC] |
| 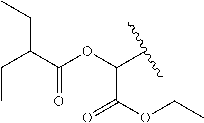 [SMILES: *C(OC(=O)C(CC)CC)C(=O)OCC] | 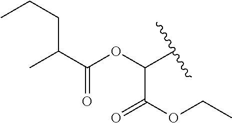 [SMILES: *C(OC(=O)C(C)CCC)C(=O)OCC] |
| 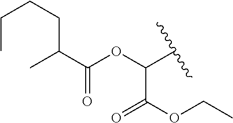 [SMILES: *C(OC(=O)C(C)CCCC)C(=O)OCC] | 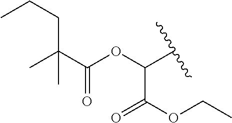 [SMILES: *C(OC(=O)C(C)(C)CCC)C(=O)OCC] |
| 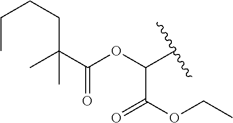 [SMILES: *C(OC(=O)C(C)(C)CCCC)C(=O)OCC] | 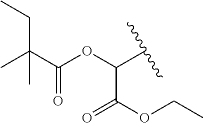 [SMILES: *C(OC(=O)C(C)(C)CC)C(=O)OCC] |
| 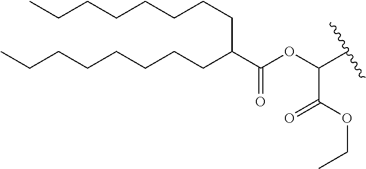 [SMILES: *C(OC(=O)C(CCCCCCCC)CCCCCCCC)C(=O)OCC] | 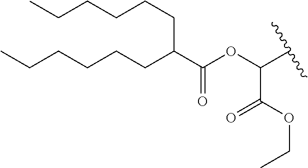 [SMILES: *C(OC(=O)C(CCCCCC)CCCCCC)C(=O)OCC] |
| 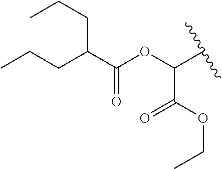 [SMILES: *C(OC(=O)C(CCC)CCC)C(=O)OCC] | 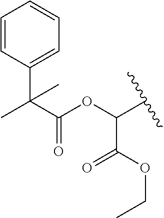 [SMILES: *C(OC(=O)C(C)(C)c1ccccc1)C(=O)OCC] |
| 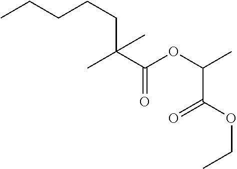 [SMILES: CCCCCC(C)(C)C(=O)OC(C)C(=O)OCC] | 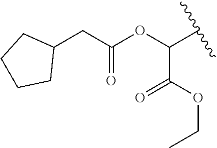 [SMILES: *C(OC(=O)CC1CCCC1)C(=O)OCC] |
| 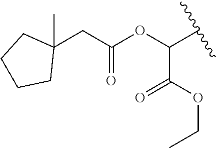 [SMILES: *C(OC(=O)CC1(C)CCCC1)C(=O)OCC] | 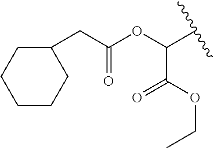 [SMILES: *C(OC(=O)CC1CCCCC1)C(=O)OCC] |
| 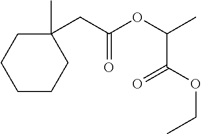 [SMILES: CCOC(=O)C(C)OC(=O)CC1(C)CCCCC1] | 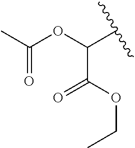 [SMILES: *C(OC(C)=O)C(=O)OCC] |
| 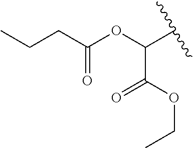 [SMILES: *C(OC(=O)CCC)C(=O)OCC] | 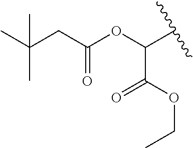 [SMILES: *C(OC(=O)CC(C)(C)C)C(=O)OCC] |
| 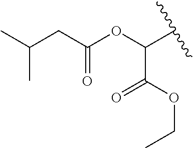 [SMILES: *C(OC(=O)CC(C)C)C(=O)OCC] | 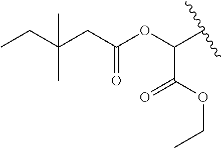 [SMILES: *C(OC(=O)CC(C)(C)CC)C(=O)OCC] |
| 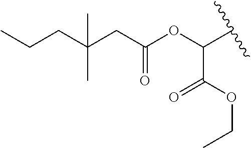 [SMILES: *C(OC(=O)CC(C)(C)CCC)C(=O)OCC] | 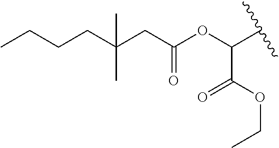 [SMILES: *C(OC(=O)CC(C)(C)CCCC)C(=O)OCC] |
| 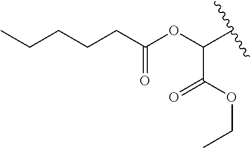 [SMILES: *C(OC(=O)CCCCC)C(=O)OCC] | 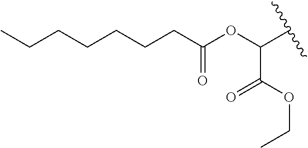 [SMILES: *C(OC(=O)CCCCCCC)C(=O)OCC] |
| 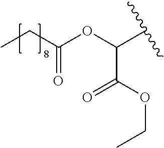 | 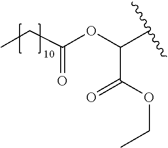 |
| 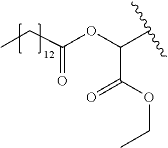 | 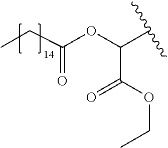 |
| 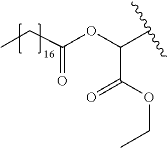 | 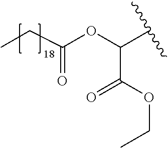 |
| 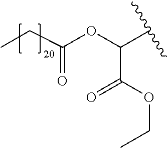 | 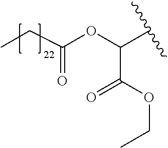 |
| 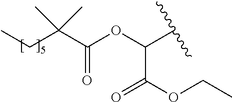 | 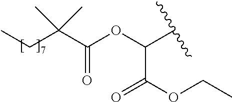 |
| 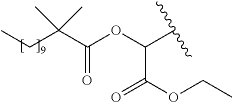 | 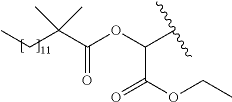 |
| 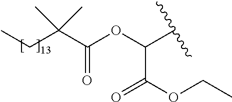 | 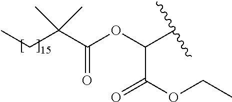 |
| 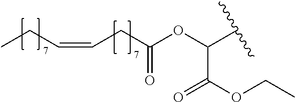 | 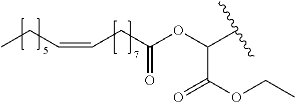 |
| 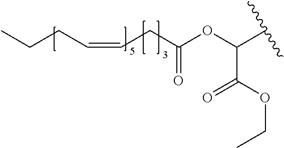 | 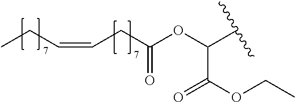 |
| 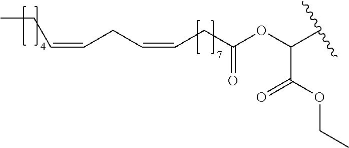 | 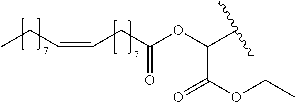 |
| 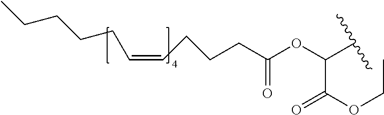 | 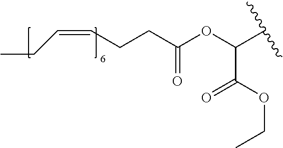 |

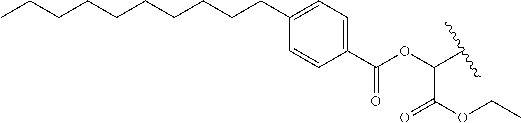 [SMILES: *C(OC(=O)c1ccc(CCCCCCCCCC)cc1)C(=O)OCC]

| | |
|---|---|
| 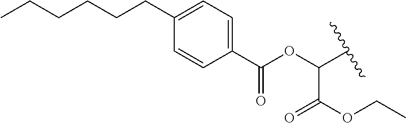 [SMILES: *C(OC(=O)c1ccc(CCCCCC)cc1)C(=O)OCC] | 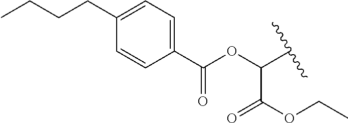 [SMILES: *C(OC(=O)c1ccc(CCCC)cc1)C(=O)OCC] |
| 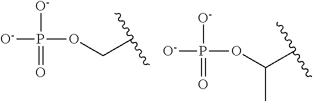 | 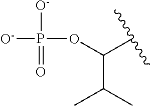 [SMILES: *C(OP(=O)([O-])[O-])C(C)C] |
| 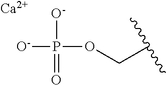 [SMILES: [Ca+2] ; *COP(=O)([O-])[O-]] | 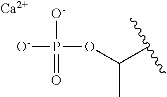 |
| 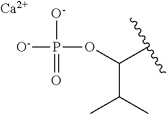 | 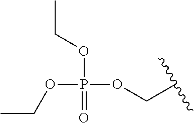 [SMILES: *COP(=O)(OCC)OCC] |
| 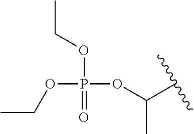 [SMILES: *C(C)OP(=O)(OCC)OCC] | 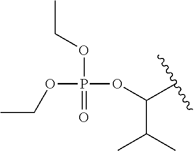 [SMILES: *C(OP(=O)(OCC)OCC)C(C)C] |
| 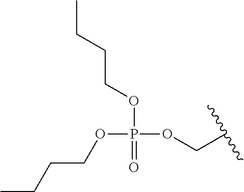 [SMILES: *COP(=O)(OCCCC)OCCCC] | 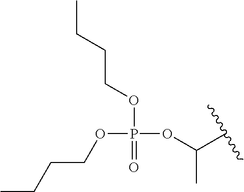 [SMILES: *C(C)OP(=O)(OCCCC)OCCCC] |
| 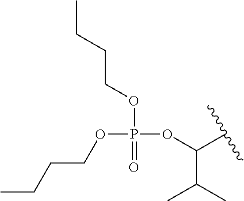 [SMILES: *C(OP(=O)(OCCCC)OCCCC)C(C)C] | 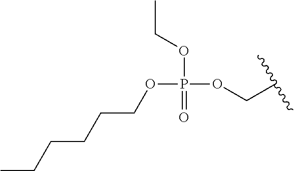 [SMILES: *COP(=O)(OCC)OCCCCCC] |
| 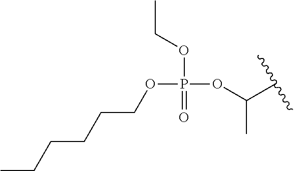 [SMILES: *C(C)OP(=O)(OCC)OCCCCCC] | 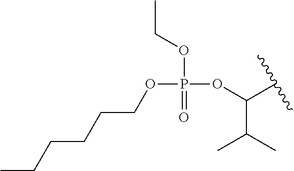 [SMILES: *C(OP(=O)(OCC)OCCCCCC)C(C)C] |
| 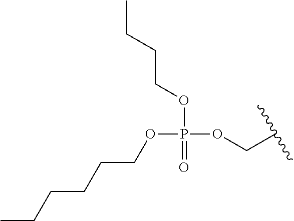 [SMILES: *COP(=O)(OCCCC)OCCCCCC] | 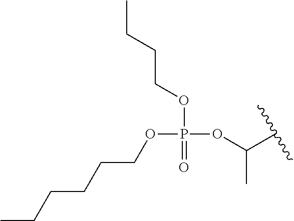 [SMILES: *C(C)OP(=O)(OCCCC)OCCCCCC] |
| 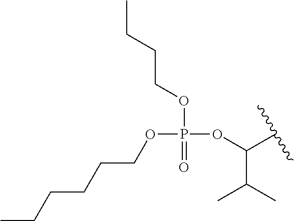 [SMILES: *C(OP(=O)(OCCCC)OCCCCCC)C(C)C] | 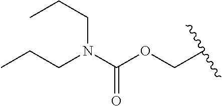 [SMILES: *COC(=O)N(CCC)CCC] |
| 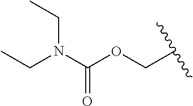 [SMILES: *COC(=O)N(CC)CC] | 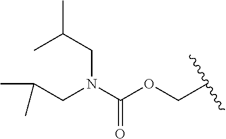 [SMILES: *COC(=O)N(CC(C)C)CC(C)C] |
| 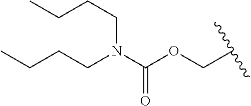 [SMILES: *COC(=O)N(CCCC)CCCC] | 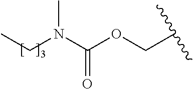 |
| 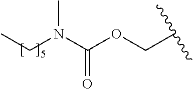 | 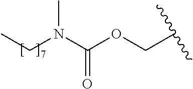 |
| 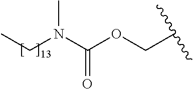 | 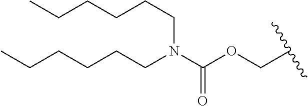 [SMILES: *COC(=O)N(CCCCCC)CCCCCC] |
| 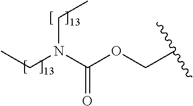 | 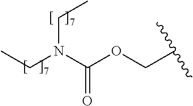 |
| 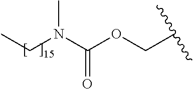 | 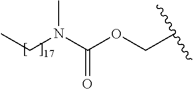 |
| 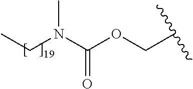 | 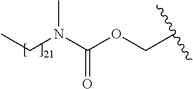 |
| 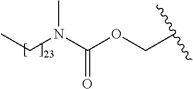 | 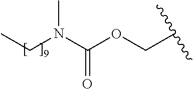 |
| 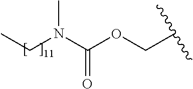 | 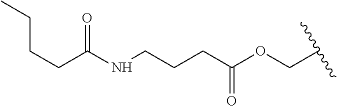 [SMILES: *COC(=O)CCCNC(=O)CCCC] |
| 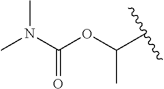 [SMILES: *C(C)OC(=O)N(C)C] | 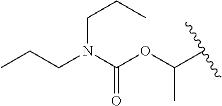 [SMILES: *C(C)OC(=O)N(CCC)CCC] |
| 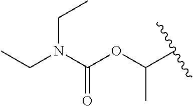 [SMILES: *C(C)OC(=O)N(CC)CC] | 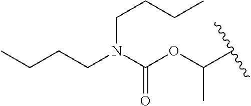 [SMILES: *C(C)OC(=O)N(CCCC)CCCC] |
| 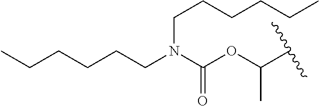 [SMILES: *C(C)OC(=O)N(CCCCCC)CCCCCC] | 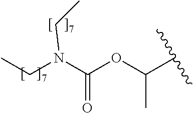 |
| 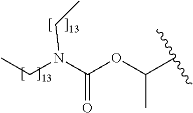 | 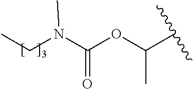 |
| 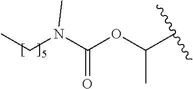 | 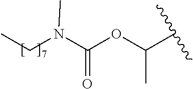 |
| 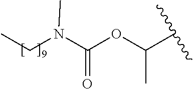 | 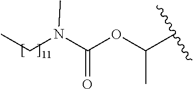 |
| 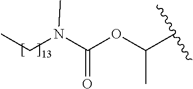 | 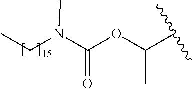 |
| 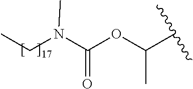 | 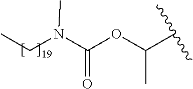 |
| 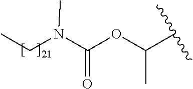 | 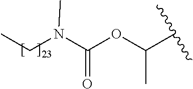 |
| 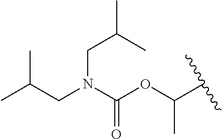 [SMILES: *C(C)OC(=O)N(CC(C)C)CC(C)C] | 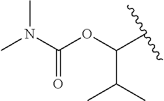 [SMILES: *C(OC(=O)N(C)C)C(C)C] |
| 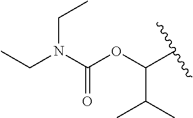 [SMILES: *C(OC(=O)N(CC)CC)C(C)C] | 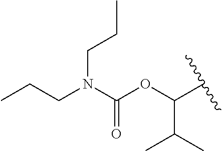 [SMILES: *C(OC(=O)N(CCC)CCC)C(C)C] |
| 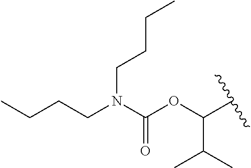 [SMILES: *C(OC(=O)N(CCCC)CCCC)C(C)C] | 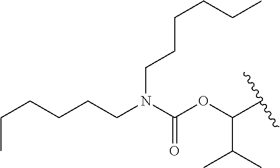 [SMILES: *C(OC(=O)N(CCCCCC)CCCCCC)C(C)C] |
| 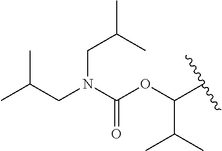 [SMILES: *C(OC(=O)N(CC(C)C)CC(C)C)C(C)C] | 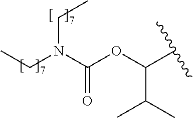 |
| 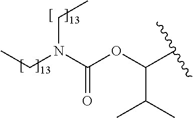 | 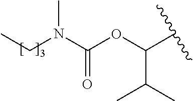 |
| 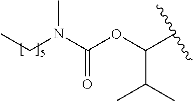 | 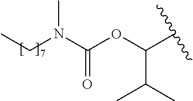 |
| 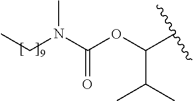 | 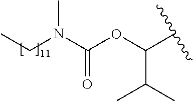 |
| 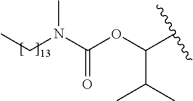 | 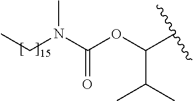 |
| 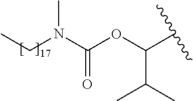 | 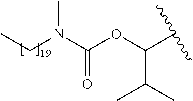 |
| 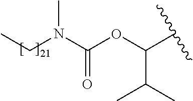 | 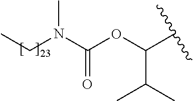 |
| 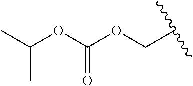 [SMILES: *COC(=O)OC(C)C] | 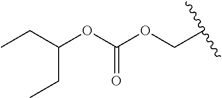 [SMILES: *COC(=O)OC(CC)CC] |
| 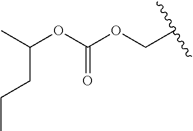 [SMILES: *COC(=O)OC(C)CCC] | 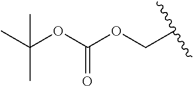 [SMILES: *COC(=O)OC(C)(C)C] |
| 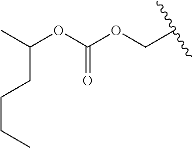 [SMILES: *COC(=O)OC(C)CCCC] | 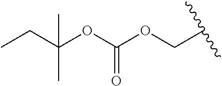 [SMILES: *COC(=O)OC(C)(C)CC] |
| 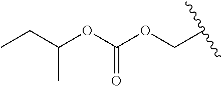 [SMILES: *COC(=O)OC(C)CC] | 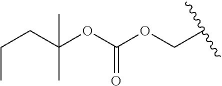 [SMILES: *COC(=O)OC(C)(C)CCC] |
| 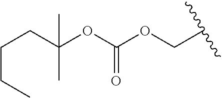 [SMILES: *COC(=O)OC(C)(C)CCCC] | 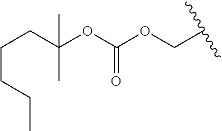 [SMILES: *COC(=O)OC(C)(C)CCCCC] |
| 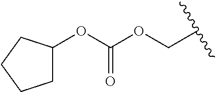 [SMILES: *COC(=O)OC1CCCC1] | 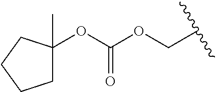 [SMILES: *COC(=O)OC1(C)CCCC1] |
| 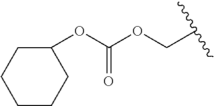 [SMILES: *COC(=O)OC1CCCCC1] | 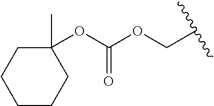 [SMILES: *COC(=O)OC1(C)CCCCC1] |
| 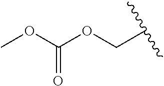 [SMILES: *COC(=O)OC] | 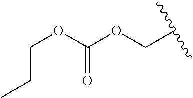 [SMILES: *COC(=O)OCCC] |
| 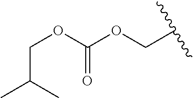 [SMILES: *COC(=O)OCC(C)C] | 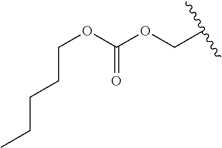 [SMILES: *COC(=O)OCCCCC] |
| 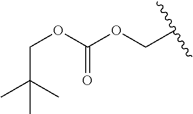 [SMILES: *COC(=O)OCC(C)(C)C] | 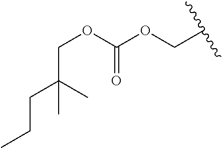 [SMILES: *COC(=O)OCC(C)(C)CCC] |
| 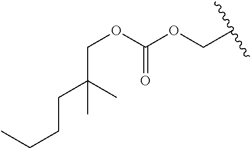 [SMILES: *COC(=O)OCC(C)(C)CCCC] | 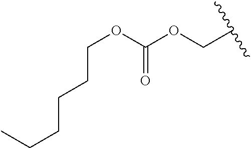 [SMILES: *COC(=O)OCCCCCC] |
| 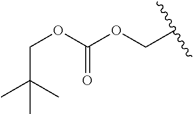 [SMILES: *COC(=O)OCC(C)(C)C] | 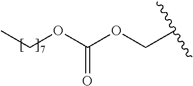 |
| 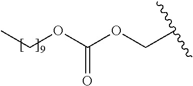 | 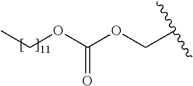 |
| 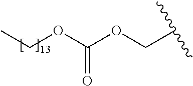 | 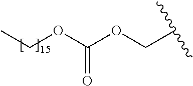 |
| 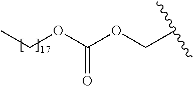 | 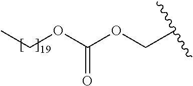 |
| 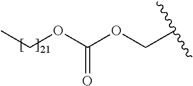 | 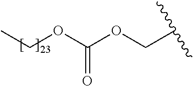 |

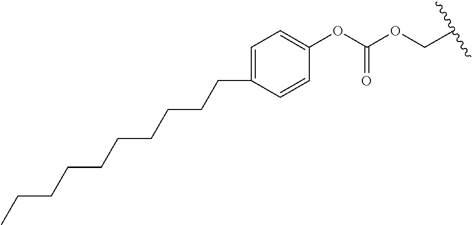 [SMILES: *COC(=O)Oc1ccc(CCCCCCCCCC)cc1]

| | |
|---|---|
| 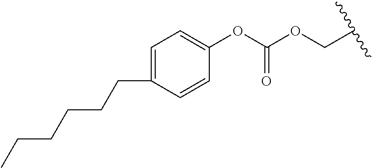 [SMILES: *COC(=O)Oc1ccc(CCCCCC)cc1] | 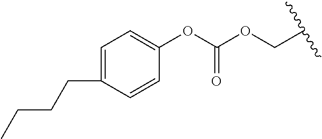 [SMILES: *COC(=O)Oc1ccc(CCCC)cc1] |
| 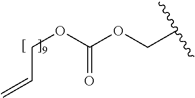 | 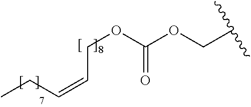 |
| 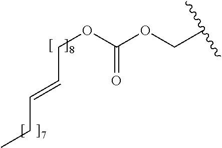 | 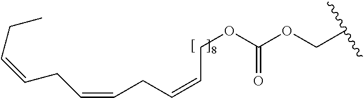 |
| 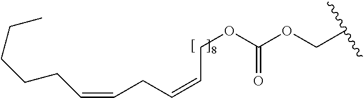 | 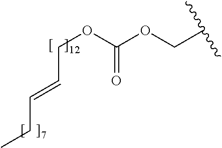 |
| 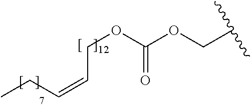 | 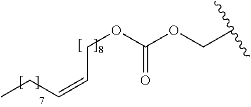 |
| 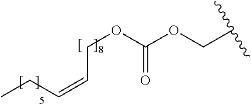 | 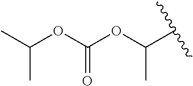 [SMILES: *C(C)OC(=O)OC(C)C] |
| 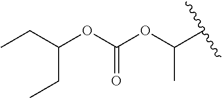 [SMILES: *C(C)OC(=O)OC(CC)CC] | 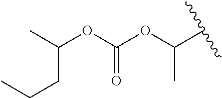 [SMILES: *C(C)OC(=O)OC(C)CCC] |
| 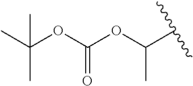 [SMILES: *C(C)OC(=O)OC(C)(C)C] | 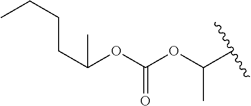 [SMILES: *C(C)OC(=O)OC(C)CCCC] |
| 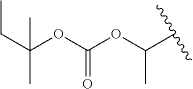 [SMILES: *C(C)OC(=O)OC(C)(C)CC] | 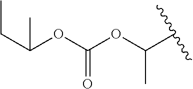 [SMILES: *C(C)OC(=O)OC(C)CC] |
| 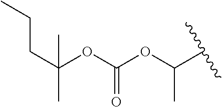 [SMILES: *C(C)OC(=O)OC(C)(C)CCC] | 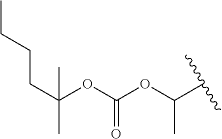 [SMILES: *C(C)OC(=O)OC(C)(C)CCCC] |
| 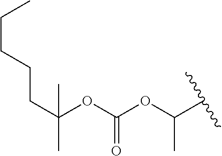 [SMILES: *C(C)OC(=O)OC(C)(C)CCCCC] | 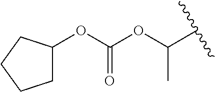 [SMILES: *C(C)OC(=O)OC1CCCC1] |
| 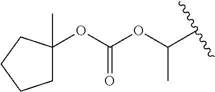 [SMILES: *C(C)OC(=O)OC1(C)CCCC1] | 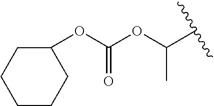 [SMILES: *C(C)OC(=O)OC1CCCCC1] |
| 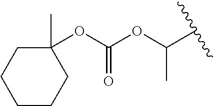 [SMILES: *C(C)OC(=O)OC1(C)CCCCC1] | 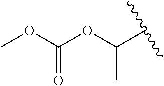 [SMILES: *C(C)OC(=O)OC] |
| 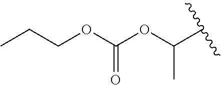 [SMILES: *C(C)OC(=O)OCCC] | 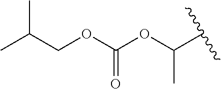 [SMILES: *C(C)OC(=O)OCC(C)C] |
| 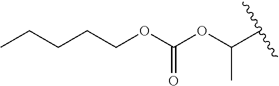 [SMILES: *C(C)OC(=O)OCCCCC] | 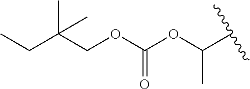 [SMILES: *C(C)OC(=O)OCC(C)(C)CC] |
| 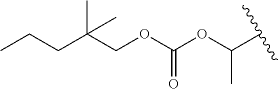 [SMILES: *C(C)OC(=O)OCC(C)(C)CCC] | 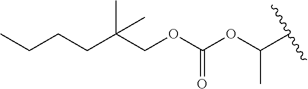 [SMILES: *C(C)OC(=O)OCC(C)(C)CCCC] |
| 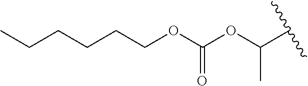 [SMILES: *C(C)OC(=O)OCCCCCC] | 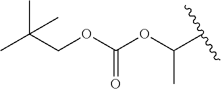 [SMILES: *C(C)OC(=O)OCC(C)(C)C] |
| 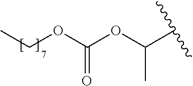 | 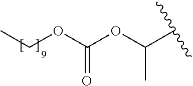 |
| 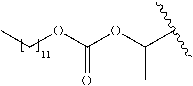 | 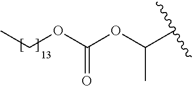 |
| 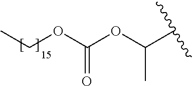 | 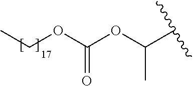 |
| 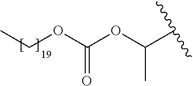 | 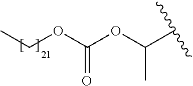 |
| 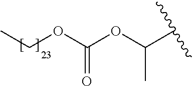 | 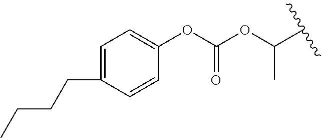 [SMILES: *C(C)OC(=O)Oc1ccc(CCCC)cc1] |

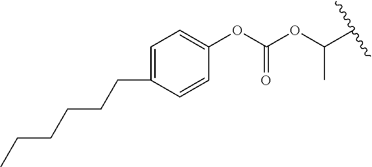 [SMILES: *C(C)OC(=O)Oc1ccc(CCCCCC)cc1]

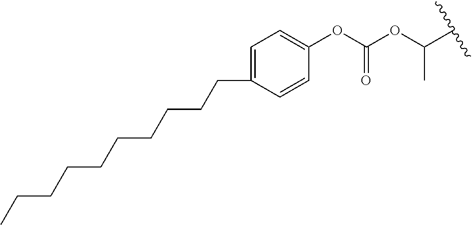 [SMILES: *C(C)OC(=O)Oc1ccc(CCCCCCCCCC)cc1]

| | |
|---|---|
| 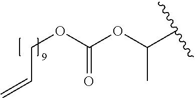 | 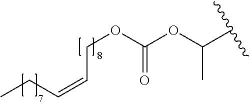 |
| 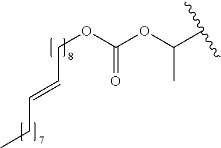 | 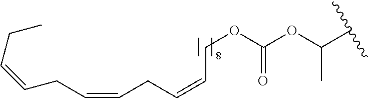 |
| 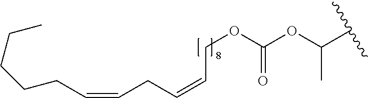 | 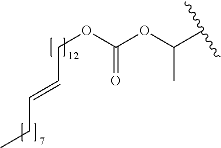 |
| 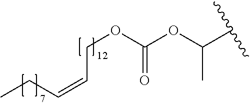 | 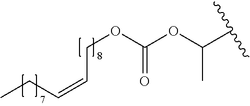 |
| 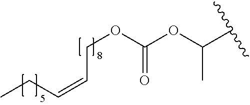 | 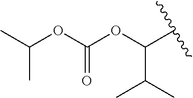 [SMILES: *C(OC(=O)OC(C)C)C(C)C] |
| 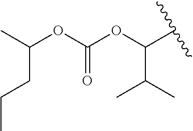 [SMILES: *C(OC(=O)OC(C)CCC)C(C)C] | 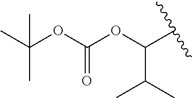 [SMILES: *C(OC(=O)OC(C)(C)C)C(C)C] |
| 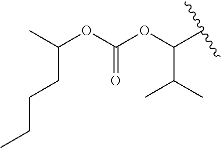 [SMILES: *C(OC(=O)OC(C)CCCC)C(C)C] | 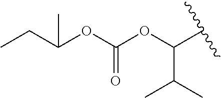 [SMILES: *C(OC(=O)OC(C)CC)C(C)C] |
| 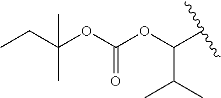 [SMILES: *C(OC(=O)OC(C)(C)CC)C(C)C] | 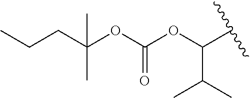 [SMILES: *C(OC(=O)OC(C)(C)CCC)C(C)C] |
| 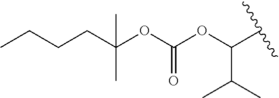 [SMILES: *C(OC(=O)OC(C)(C)CCCC)C(C)C] | 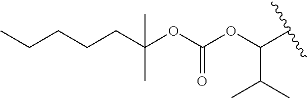 [SMILES: *C(OC(=O)OC(C)(C)CCCCC)C(C)C] |
| 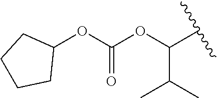 [SMILES: *C(OC(=O)OC1CCCC1)C(C)C] | 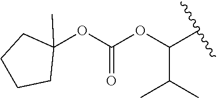 [SMILES: *C(OC(=O)OC1(C)CCCC1)C(C)C] |
| 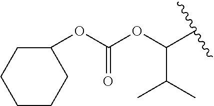 [SMILES: *C(OC(=O)OC1CCCCC1)C(C)C] | 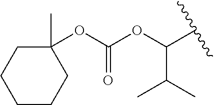 [SMILES: *C(OC(=O)OC1(C)CCCCC1)C(C)C] |
| 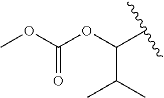 [SMILES: *C(OC(=O)OC)C(C)C] | 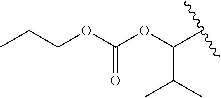 [SMILES: *C(OC(=O)OCCC)C(C)C] |
| 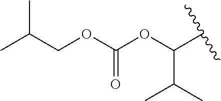 [SMILES: *C(OC(=O)OCC(C)C)C(C)C] | 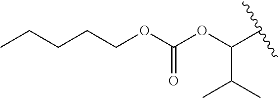 [SMILES: *C(OC(=O)OCCCCC)C(C)C] |
| 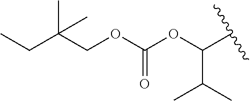 [SMILES: *C(OC(=O)OCC(C)(C)CC)C(C)C] | 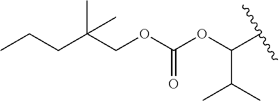 [SMILES: *C(OC(=O)OCC(C)(C)CCC)C(C)C] |
| 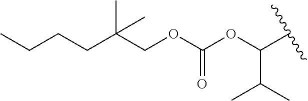 [SMILES: *C(OC(=O)OCC(C)(C)CCCC)C(C)C] | 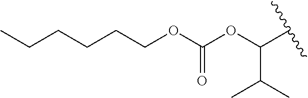 [SMILES: *C(OC(=O)OCCCCCC)C(C)C] |
| 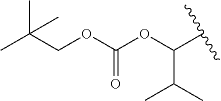 [SMILES: *C(OC(=O)OCC(C)(C)C)C(C)C] | 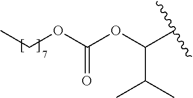 |
| 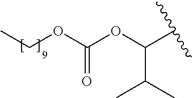 | 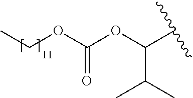 |
| 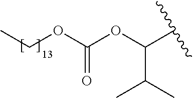 | 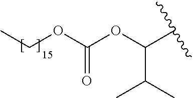 |
| 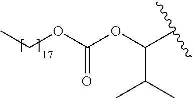 | 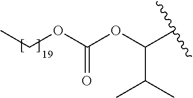 |
| 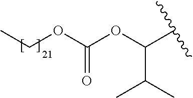 | 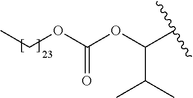 |
| 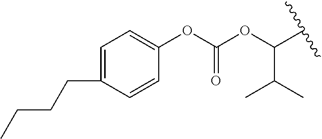 [SMILES: *C(OC(=O)Oc1ccc(CCCC)cc1)C(C)C] | 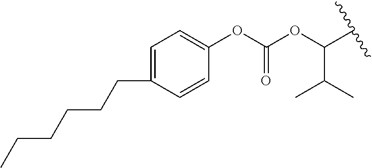 [SMILES: *C(OC(=O)Oc1ccc(CCCCCC)cc1)C(C)C] |
| 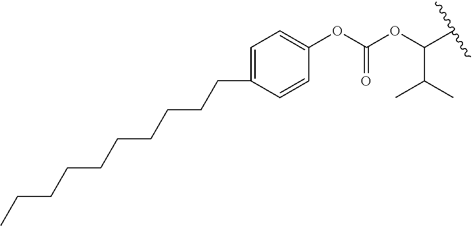 [SMILES: *C(OC(=O)Oc1ccc(CCCCCCCCCC)cc1)C(C)C] | 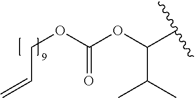 |
| 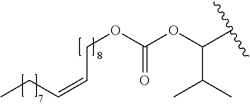 | 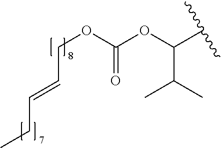 |
| 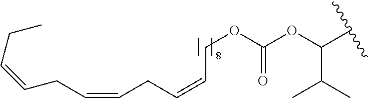 | 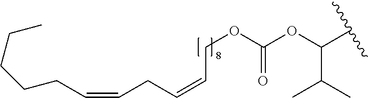 |
| 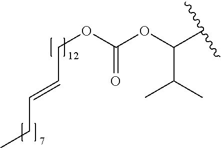 | 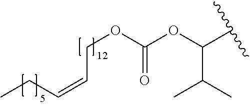 |
| 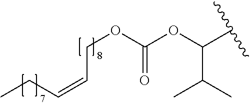 | 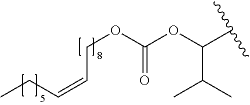 |
| 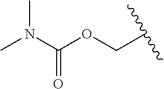 [SMILES: *COC(=O)N(C)C] | 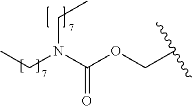 |

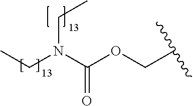

In a 46^{th} aspect, provided herein is a compound, or pharmaceutically acceptable salt thereof, having a structure provided in Formula (I-15),

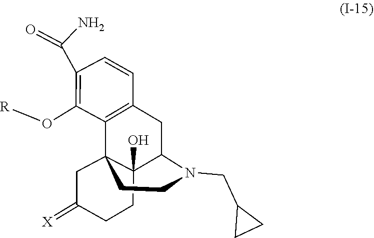

wherein X is ═O, R is R⁴—O—CH(R³)— and R³ and R⁴ are any of the pair of R³/R⁴ groups recited in Table 5. Alternatively, X is ═CH₂, R is R⁴—O—CH(R³)— and R³ and R⁴ are any of the pair of R³/R⁴ groups recited in Table 5. Alternatively, X is ═CH₂ and R is an R⁴ group recited in Table 5. Alternatively, X is ═O and R is an R⁴ group recited for R⁴ in Table 5. Alternatively, X is ═O, R is R⁴OCH₂— and R⁴ is an R⁴ group recited in Table 5. Alternatively, X is ═CH₂, R is R⁴OCH₂—, and R⁴ an R⁴ group recited in Table 5.

In a 47^{th} aspect, provided herein is a compound, or pharmaceutically acceptable salt thereof, having a structure provided in Formula (II-15),

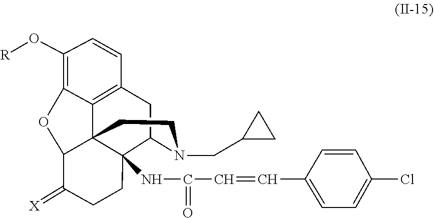

wherein X is ═O and R is R⁴—O—CH(R³)—, R³ and R⁴ are any of the pair of R³/R⁴ groups recited in Table 5. Alternatively, X is ═CH₂, R is R⁴—O—CH(R³)— and R³ and R⁴ are any of the pair of R³/R⁴ groups recited in Table 5. Alternatively, X is ═O and R is an R⁴ group recited in Table 5. Alternatively, X is ═CH₂ and R is an R⁴ group recited in Table 5. Alternatively, X is ═O, R is R⁴OCH₂— and R⁴ is an R⁴ group recited in Table 5. Alternatively, X is ═CH₂, R is R⁴OCH₂—, and R⁴ an R⁴ group recited in Table 5.

In a 48^{th} aspect, provided herein is a compound, or pharmaceutically acceptable salt thereof, having a structure provided in Formula (III-15),

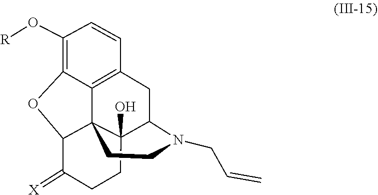

wherein X is ═O and R is R⁴—O—CH(R³)—, R³ and R⁴ are any of the pair of R³/R⁴ groups recited in Table 5. Alternatively, X is ═CH₂, R is R⁴—O—CH(R³)— and R³ and R⁴ are any of the pair of R³/R⁴ groups recited in Table 5. Alternatively, X is ═CH₂ and R is an R⁴ group recited in Table 5. Alternatively, X is ═O and R is an R⁴ group recited in Table 5. Alternatively, X is ═O, R is R⁴OCH₂— and R⁴ is an R⁴ group recited in Table 5. Alternatively, X is ═CH₂, R is R⁴OCH₂—, and R⁴ an R⁴ group recited in Table 5.

TABLE 5

| R³ | R⁴ |
|---|---|
| H | Me |
| H | —C₇H₁₅ |
| H | —C₉H₁₉ |
| H | —C₁₁H₂₃ |
| H | —C₁₃H₂₇ |
| H | —C₁₅H₃₁ |
| H | —C₁₇H₃₅ |
| H | 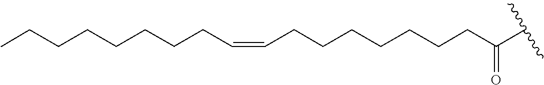 [SMILES: *C(=O)CCCCCCC/C=C\CCCCCCCC] |
| H | 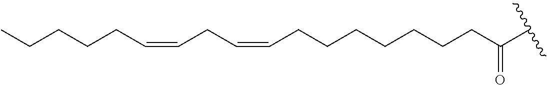 [SMILES: *C(=O)CCCCCCC/C=C\C/C=C\CCCCC] |
| H | 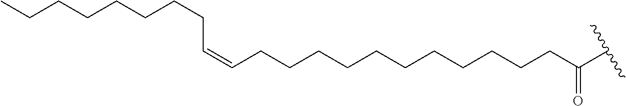 [SMILES: *C(=O)CCCCCCCCCCC/C=C\CCCCCCCC] |
| H | 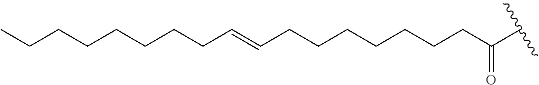 [SMILES: *C(=O)CCCCCCC/C=C/CCCCCCCC] |
| H | 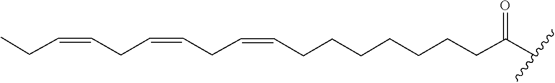 [SMILES: *C(=O)CCCCCCC/C=C\C/C=C\C/C=C\CC] |
| H | tBu |
| H | phenyl |
| H | 4-chlorophenyl |
| H | 3,4-dichlorophenyl |
| H | 4-methylphenyl |
| H | 4-methoxyphenyl |
| Me | Me |
| Me | —C₇H₁₅ |
| Me | —C₉H₁₉ |
| Me | —C₁₁H₂₃ |
| Me | —C₁₃H₂₇ |
| Me | —C₁₅H₃₁ |
| Me | —C₁₇H₃₅ |
| Me | 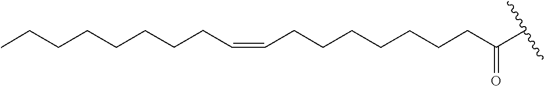 [SMILES: *C(=O)CCCCCCC/C=C\CCCCCCCC] |
| Me | 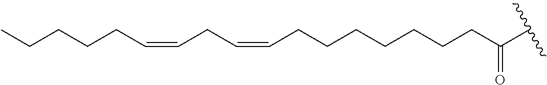 [SMILES: *C(=O)CCCCCCC/C=C\C/C=C\CCCCC] |
| Me | 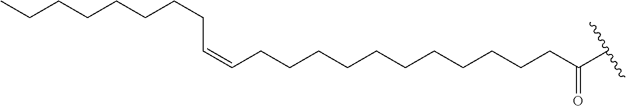 [SMILES: *C(=O)CCCCCCCCCCC/C=C\CCCCCCCC] |
| Me | 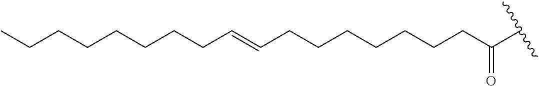 [SMILES: *C(=O)CCCCCCC/C=C/CCCCCCCC] |
| Me | 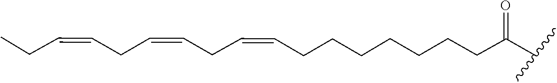 [SMILES: *C(=O)CCCCCCC/C=C\C/C=C\C/C=C\CC] |
| Me | tBu |
| Me | phenyl |
| Me | 4-chlorophenyl |
| Me | 3,4-dichlorophenyl |
| Me | 4-methylphenyl |
| Me | 4-methoxyphenyl |
| tBu | Me |
| tBu | —C₇H₁₅ |
| tBu | —C₉H₁₉ |
| tBu | —C₁₁H₂₃ |
| tBu | —C₁₃H₂₇ |
| tBu | —C₁₅H₃₁ |
| tBu | —C₁₇H₃₅ |
| tBu | 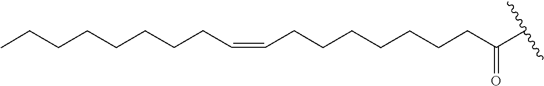 [SMILES: *C(=O)CCCCCCC/C=C\CCCCCCCC] |
| tBu | 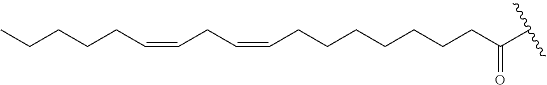 [SMILES: *C(=O)CCCCCCC/C=C\C/C=C\CCCCC] |
| tBu | 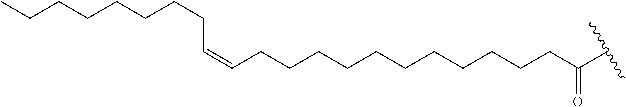 [SMILES: *C(=O)CCCCCCCCCCC/C=C\CCCCCCCC] |
| tBu | 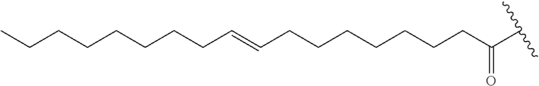 [SMILES: *C(=O)CCCCCCC/C=C/CCCCCCCC] |
| tBu | 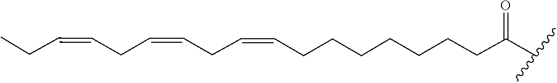 [SMILES: *C(=O)CCCCCCC/C=C\C/C=C\C/C=C\CC] |
| tBu | phenyl |
| tBu | tBu |
| tBu | 4-chlorophenyl |
| tBu | 3,4-dichlorophenyl |
| tBu | 4-methylphenyl |
| tBu | 4-methoxyphenyl |
| —C₇H₁₅ | Me |
| —C₇H₁₅ | —C₇H₁₅ |
| —C₇H₁₅ | —C₉H₁₉ |
| —C₇H₁₅ | —C₁₁H₂₃ |
| —C₇H₁₅ | —C₁₃H₂₇ |
| —C₇H₁₅ | —C₁₅H₃₁ |
| —C₇H₁₅ | —C₁₇H₃₅ |
| —C₇H₁₅ | 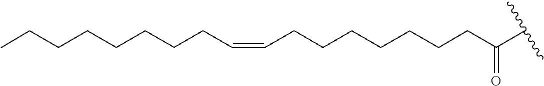 [SMILES: *C(=O)CCCCCCC/C=C\CCCCCCCC] |
| —C₇H₁₅ | 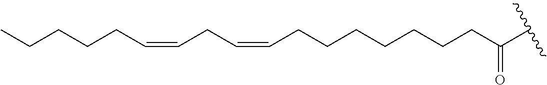 [SMILES: *C(=O)CCCCCCC/C=C\C/C=C\CCCCC] |
| —C₇H₁₅ | 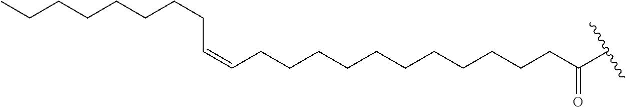 [SMILES: *C(=O)CCCCCCCCCCC/C=C\CCCCCCCC] |
| —C₇H₁₅ | 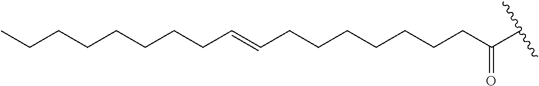 [SMILES: *C(=O)CCCCCCC/C=C/CCCCCCCC] |
| —C₇H₁₅ | 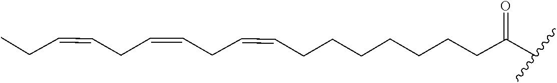 [SMILES: *C(=O)CCCCCCC/C=C\C/C=C\C/C=C\CC] |
| —C₇H₁₅ | tBu |
| —C₇H₁₅ | phenyl |
| —C₇H₁₅ | 4-chlorophenyl |
| —C₇H₁₅ | 3,4-dichlorophenyl |
| —C₇H₁₅ | 4-methylphenyl |
| —C₇H₁₅ | 4-methoxyphenyl |
| —C₉H₁₉ | Me |
| —C₉H₁₉ | —C₇H₁₅ |
| —C₉H₁₉ | —C₉H₁₉ |
| —C₉H₁₉ | —C₁₁H₂₃ |
| —C₉H₁₉ | —C₁₃H₂₇ |
| —C₉H₁₉ | —C₁₅H₃₁ |
| —C₉H₁₉ | —C₁₇H₃₅ |
| —C₉H₁₉ | 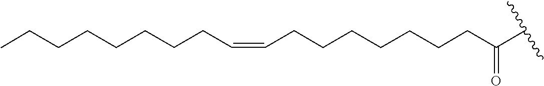 [SMILES: *C(=O)CCCCCCC/C=C\CCCCCCCC] |
| —C₉H₁₉ | 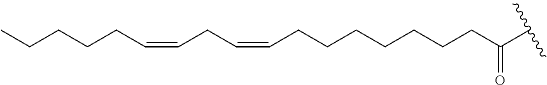 [SMILES: *C(=O)CCCCCCC/C=C\C/C=C\CCCCC] |
| —C₉H₁₉ | 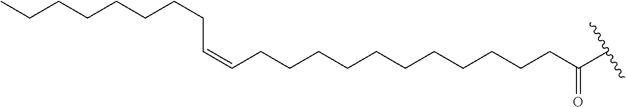 [SMILES: *C(=O)CCCCCCCCCCC/C=C\CCCCCCCC] |
| —C₉H₁₉ | 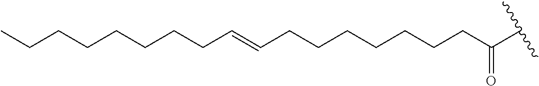 [SMILES: *C(=O)CCCCCCC/C=C/CCCCCCCC] |
| —C₉H₁₉ | 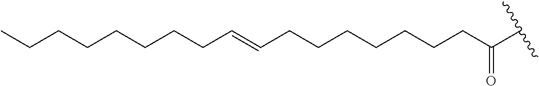 [SMILES: *C(=O)CCCCCCC/C=C/CCCCCCCC] |
| —C₉H₁₉ | 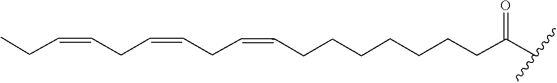 [SMILES: *C(=O)CCCCCCC/C=C\C/C=C\C/C=C\CC] |
| —C₉H₁₉ | tBu |
| —C₉H₁₉ | phenyl |
| —C₉H₁₉ | 4-chlorophenyl |
| —C₉H₁₉ | 3,4-dichlorophenyl |
| —C₉H₁₉ | 4-methylphenyl |
| —C₉H₁₉ | 4-methoxyphenyl |
| —C₁₁H₂₃ | Me |
| —C₁₁H₂₃ | —C₇H₁₅ |
| —C₁₁H₂₃ | —C₉H₁₉ |
| —C₁₁H₂₃ | —C₁₁H₂₃ |
| —C₁₁H₂₃ | —C₁₃H₂₇ |
| —C₁₁H₂₃ | —C₁₅H₃₁ |
| —C₁₁H₂₃ | —C₁₇H₃₅ |
| —C₁₁H₂₃ | 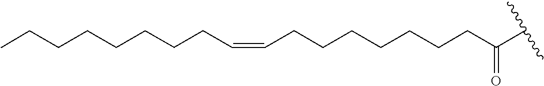 [SMILES: *C(=O)CCCCCCC/C=C\CCCCCCCC] |
| —C₁₁H₂₃ | 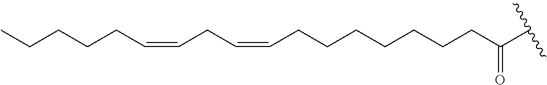 [SMILES: *C(=O)CCCCCCC/C=C\C/C=C\CCCCC] |
| —C₁₁H₂₃ | 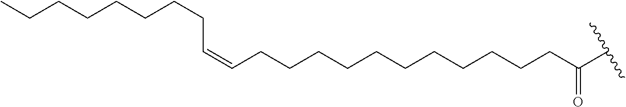 [SMILES: *C(=O)CCCCCCCCCCC/C=C\CCCCCCCC] |
| —C₁₁H₂₃ | 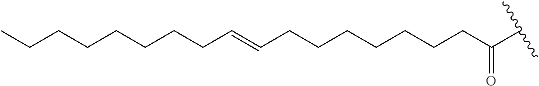 [SMILES: *C(=O)CCCCCCC/C=C/CCCCCCCC] |
| —C₁₁H₂₃ | 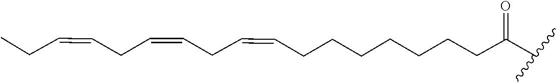 [SMILES: *C(=O)CCCCCCC/C=C\C/C=C\C/C=C\CC] |
| —C₁₁H₂₃ | tBu |
| —C₁₁H₂₃ | phenyl |
| —C₁₁H₂₃ | 4-chlorophenyl |
| —C₁₁H₂₃ | 3,4-dichlorophenyl |
| —C₁₁H₂₃ | 4-methylphenyl |
| —C₁₁H₂₃ | 4-methoxyphenyl |
| —C₁₃H₂₇ | Me |
| —C₁₃H₂₇ | —C₇H₁₅ |
| —C₁₃H₂₇ | —C₉H₁₉ |
| —C₁₃H₂₇ | —C₁₁H₂₃ |
| —C₁₃H₂₇ | —C₁₃H₂₇ |
| —C₁₃H₂₇ | —C₁₅H₃₁ |
| —C₁₃H₂₇ | —C₁₇H₃₅ |
| —C₁₃H₂₇ | 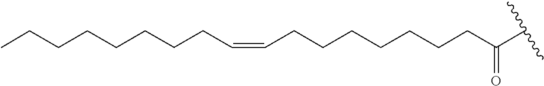 [SMILES: *C(=O)CCCCCCC/C=C\CCCCCCCC] |
| —C₁₃H₂₇ | 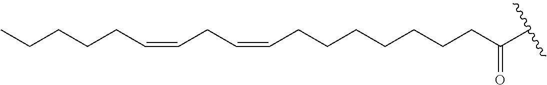 [SMILES: *C(=O)CCCCCCC/C=C\C/C=C\CCCCC] |
| —C₁₃H₂₇ | 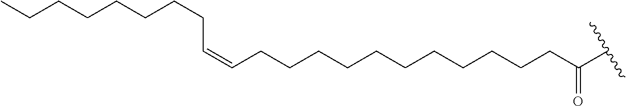 [SMILES: *C(=O)CCCCCCCCCCC/C=C\CCCCCCCC] |
| —C₁₃H₂₇ | 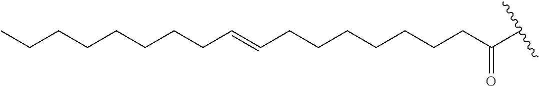 [SMILES: *C(=O)CCCCCCC/C=C/CCCCCCCC] |
| —C₁₃H₂₇ | 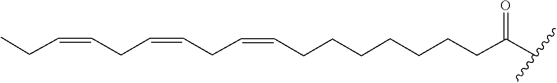 [SMILES: *C(=O)CCCCCCC/C=C\C/C=C\C/C=C\CC] |
| —C₁₃H₂₇ | tBu |
| —C₁₃H₂₇ | phenyl |
| —C₁₃H₂₇ | 4-chlorophenyl |
| —C₁₃H₂₇ | 3,4-dichlorophenyl |
| —C₁₃H₂₇ | 4-methylphenyl |
| —C₁₃H₂₇ | 4-methoxyphenyl |
| —C₁₅H₃₁ | Me |
| —C₁₅H₃₁ | —C₇H₁₅ |
| —C₁₅H₃₁ | —C₉H₁₉ |
| —C₁₅H₃₁ | —C₁₁H₂₃ |
| —C₁₅H₃₁ | —C₁₃H₂₇ |
| —C₁₅H₃₁ | —C₁₅H₃₁ |
| —C₁₅H₃₁ | —C₁₇H₃₅ |
| —C₁₅H₃₁ | 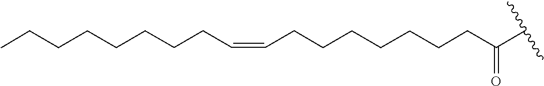 [SMILES: *C(=O)CCCCCCC/C=C\CCCCCCCC] |
| —C₁₅H₃₁ | 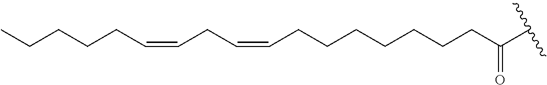 [SMILES: *C(=O)CCCCCCC/C=C\C/C=C\CCCCC] |
| —C₁₅H₃₁ | 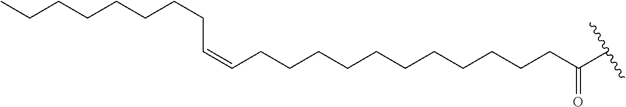 [SMILES: *C(=O)CCCCCCCCCCC/C=C\CCCCCCCC] |
| —C₁₅H₃₁ | 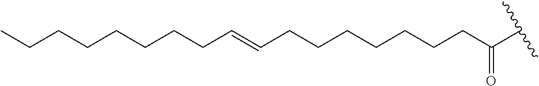 [SMILES: *C(=O)CCCCCCC/C=C/CCCCCCCC] |
| —C₁₅H₃₁ | 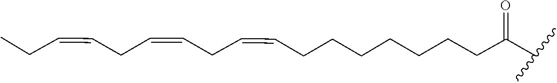 [SMILES: *C(=O)CCCCCCC/C=C\C/C=C\C/C=C\CC] |
| —C₁₅H₃₁ | tBu |
| —C₁₅H₃₁ | phenyl |
| —C₁₅H₃₁ | 4-chlorophenyl |
| —C₁₅H₃₁ | 3,4-dichlorophenyl |
| —C₁₅H₃₁ | 4-methylphenyl |
| —C₁₅H₃₁ | 4-methoxyphenyl |
| —C₁₇H₃₅ | Me |
| —C₁₇H₃₅ | —C₇H₁₅ |
| —C₁₇H₃₅ | —C₉H₁₉ |
| —C₁₇H₃₅ | —C₁₁H₂₃ |
| —C₁₇H₃₅ | —C₁₃H₂₇ |
| —C₁₇H₃₅ | —C₁₅H₃₁ |
| —C₁₇H₃₅ | —C₁₇H₃₅ |
| —C₁₇H₃₅ | 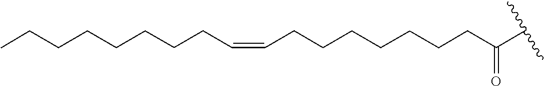 [SMILES: *C(=O)CCCCCCC/C=C\CCCCCCCC] |
| —C₁₇H₃₅ | 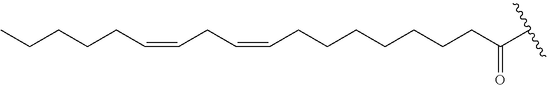 [SMILES: *C(=O)CCCCCCC/C=C\C/C=C\CCCCC] |
| —C₁₇H₃₅ | 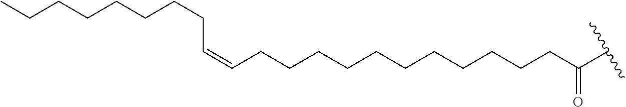 [SMILES: *C(=O)CCCCCCCCCCC/C=C\CCCCCCCC] |
| —C₁₇H₃₅ | 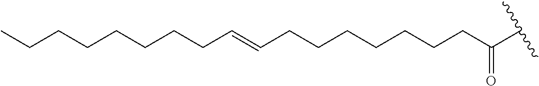 [SMILES: *C(=O)CCCCCCC/C=C/CCCCCCCC] |
| —C₁₇H₃₅ | 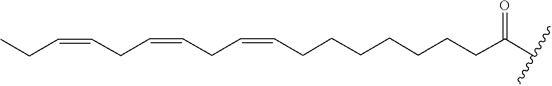 [SMILES: *C(=O)CCCCCCC/C=C\C/C=C\C/C=C\CC] |
| —C₁₇H₃₅ | tBu |
| —C₁₇H₃₅ | phenyl |
| —C₁₇H₃₅ | 4-chlorophenyl |
| —C₁₇H₃₅ | 3,4-dichlorophenyl |
| —C₁₇H₃₅ | 4-methylphenyl |
| —C₁₇H₃₅ | 4-methoxyphenyl |
| Phenyl | Me |
| Phenyl | —C₇H₁₅ |
| Phenyl | —C₉H₁₉ |
| Phenyl | —C₁₁H₂₃ |
| Phenyl | —C₁₃H₂₇ |
| Phenyl | —C₁₅H₃₁ |
| Phenyl | —C₁₇H₃₅ |
| Phenyl | 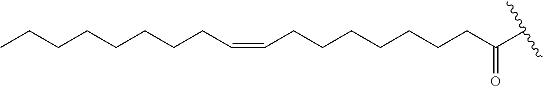 [SMILES: *C(=O)CCCCCCC/C=C\CCCCCCCC] |
| Phenyl | 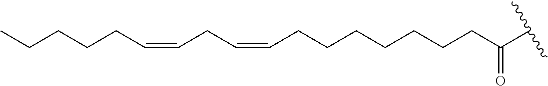 [SMILES: *C(=O)CCCCCCC/C=C\C/C=C\CCCCC] |
| Phenyl | 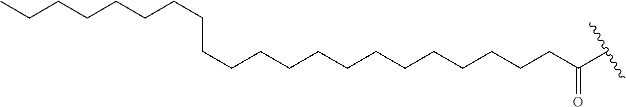 [SMILES: *C(=O)CCCCCCCCCCCCCCCCCCCCC] |
| Phenyl | 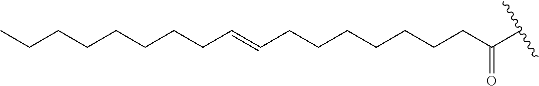 [SMILES: *C(=O)CCCCCCC/C=C/CCCCCCCC] |
| Phenyl | 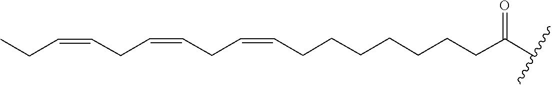 [SMILES: *C(=O)CCCCCCC/C=C\C/C=C\C/C=C\CC] |
| Phenyl | tBu |
| Phenyl | phenyl |
| Phenyl | 4-chlorophenyl |
| Phenyl | 3,4-dichlorophenyl |
| Phenyl | 4-methylphenyl |
| Phenyl | 4-methoxyphenyl |
| 4-chlorophenyl | Me |
| 4-chlorophenyl | —C₇H₁₅ |
| 4-chlorophenyl | —C₉H₁₉ |
| 4-chlorophenyl | —C₁₁H₂₃ |
| 4-chlorophenyl | —C₁₃H₂₇ |
| 4-chlorophenyl | —C₁₅H₃₁ |
| 4-chlorophenyl | —C₁₇H₃₅ |
| 4-chlorophenyl | 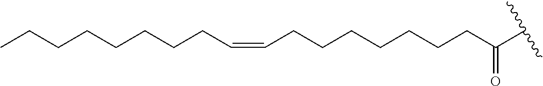 [SMILES: *C(=O)CCCCCCC/C=C\CCCCCCCC] |
| 4-chlorophenyl | 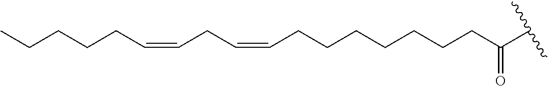 [SMILES: *C(=O)CCCCCCC/C=C\C/C=C\CCCCC] |
| 4-chlorophenyl | 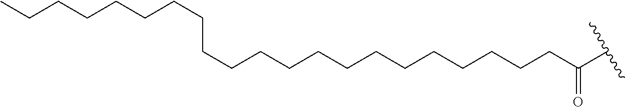 [SMILES: *C(=O)CCCCCCCCCCCCCCCCCCCCC] |
| 4-chlorophenyl | 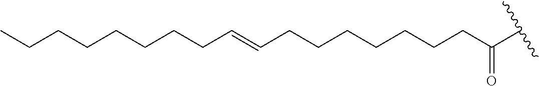 [SMILES: *C(=O)CCCCCCC/C=C/CCCCCCCC] |
| 4-chlorophenyl | 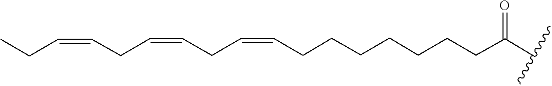 [SMILES: *C(=O)CCCCCCC/C=C\C/C=C\C/C=C\CC] |
| 4-chlorophenyl | tBu |
| 4-chlorophenyl | phenyl |
| 4-chlorophenyl | 4-chlorophenyl |
| 4-chlorophenyl | 3,4-dichlorophenyl |
| 4-chlorophenyl | 4-methylphenyl |
| 4-chlorophenyl | 4-methoxyphenyl |
| 3,4-dichlorophenyl | Me |
| 3,4-dichlorophenyl | —C₇H₁₅ |
| 3,4-dichlorophenyl | —C₉H₁₉ |
| 3,4-dichlorophenyl | —C₁₁H₂₃ |
| 3,4-dichlorophenyl | —C₁₃H₂₇ |
| 3,4-dichlorophenyl | —C₁₅H₃₁ |
| 3,4-dichlorophenyl | —C₁₇H₃₅ |
| 3,4-dichlorophenyl | 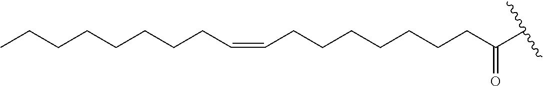 [SMILES: *C(=O)CCCCCCC/C=C\CCCCCCCC] |
| 3,4-dichlorophenyl | 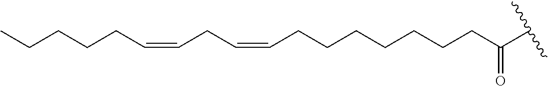 [SMILES: *C(=O)CCCCCCC/C=C\C/C=C\CCCCC] |
| 3,4-dichlorophenyl | 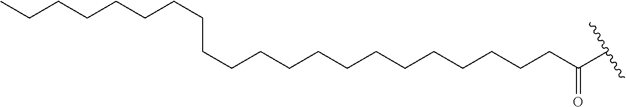 [SMILES: *C(=O)CCCCCCCCCCCCCCCCCCCCC] |
| 3,4-dichlorophenyl | 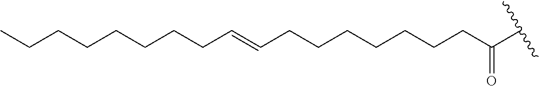 [SMILES: *C(=O)CCCCCCC/C=C/CCCCCCCC] |
| 3,4-dichlorophenyl | 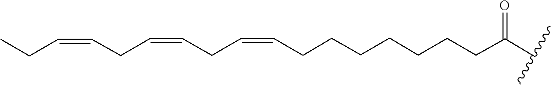 [SMILES: *C(=O)CCCCCCC/C=C\C/C=C\C/C=C\CC] |
| 3,4-dichlorophenyl | tBu |
| 3,4-dichlorophenyl | phenyl |
| 3,4-dichlorophenyl | 4-chlorophenyl |
| 3,4-dichlorophenyl | 3,4-dichlorophenyl |
| 3,4-dichlorophenyl | 4-methylphenyl |
| 3,4-dichlorophenyl | 4-methoxyphenyl |
| 4-methylphenyl | Me |
| 4-methylphenyl | —C₇H₁₅ |
| 4-methylphenyl | —C₉H₁₉ |
| 4-methylphenyl | —C₁₁H₂₃ |
| 4-methylphenyl | —C₁₃H₂₇ |
| 4-methylphenyl | —C₁₅H₃₁ |
| 4-methylphenyl | —C₁₇H₃₅ |
| 4-methylphenyl | 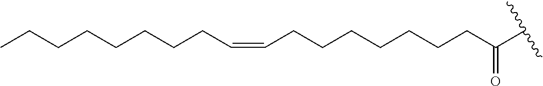 [SMILES: *C(=O)CCCCCCC/C=C\CCCCCCCC] |
| 4-methylphenyl | 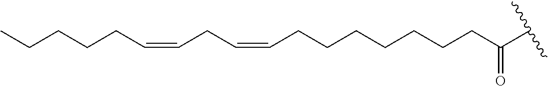 [SMILES: *C(=O)CCCCCCC/C=C\C/C=C\CCCCC] |
| 4-methylphenyl | 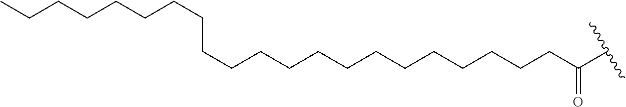 [SMILES: *C(=O)CCCCCCCCCCCCCCCCCCCCC] |
| 4-methylphenyl | 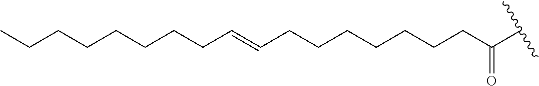 [SMILES: *C(=O)CCCCCCC/C=C/CCCCCCCC] |
| 4-methylphenyl | 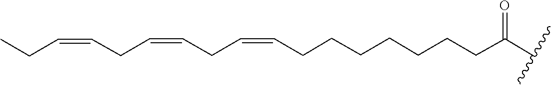 [SMILES: *C(=O)CCCCCCC/C=C\C/C=C\C/C=C\CC] |
| 4-methylphenyl | tBu |
| 4-methylphenyl | phenyl |
| 4-methylphenyl | 4-chlorophenyl |
| 4-methylphenyl | 3,4-dichlorophenyl |
| 4-methylphenyl | 4-methylphenyl |
| 4-methylphenyl | 4-methoxyphenyl |
| 4-methoxyphenyl | Me |
| 4-methoxyphenyl | —C₇H₁₅ |
| 4-methoxyphenyl | —C₉H₁₉ |
| 4-methoxyphenyl | —C₁₁H₂₃ |
| 4-methoxyphenyl | —C₁₃H₂₇ |
| 4-methoxyphenyl | —C₁₅H₃₁ |
| 4-methoxyphenyl | —C₁₇H₃₅ |
| 4-methoxyphenyl | 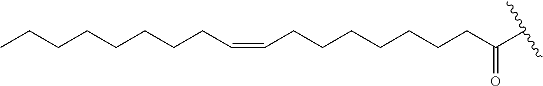 [SMILES: *C(=O)CCCCCCC/C=C\CCCCCCCC] |
| 4-methoxyphenyl | 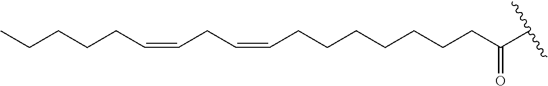 [SMILES: *C(=O)CCCCCCC/C=C\C/C=C\CCCCC] |
| 4-methoxyphenyl | 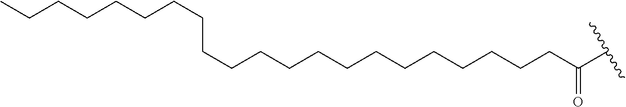 [SMILES: *C(=O)CCCCCCCCCCCCCCCCCCCCC] |
| 4-methoxyphenyl | 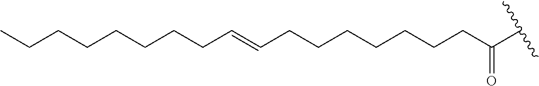 [SMILES: *C(=O)CCCCCCC/C=C/CCCCCCCC] |
| 4-methoxyphenyl | 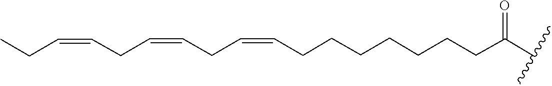 [SMILES: *C(=O)CCCCCCC/C=C\C/C=C\C/C=C\CC] |
| 4-methoxyphenyl | tBu |
| 4-methoxyphenyl | phenyl |
| 4-methoxyphenyl | 4-chlorophenyl |
| 4-methoxyphenyl | 3,4-dichlorophenyl |
| 4-methoxyphenyl | 4-methylphenyl |
| 4-methoxyphenyl | 4-methoxyphenyl |
| 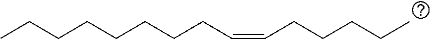 | Me |
| 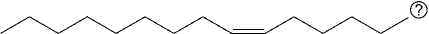 | —C₇H₁₅ |
| 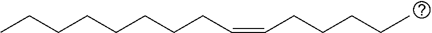 | —C₉H₁₉ |
| 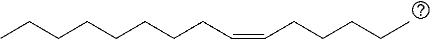 | —C₁₁H₂₃ |
| 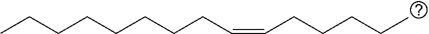 | —C₁₃H₂₇ |
| 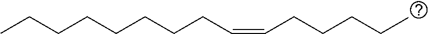 | —C₁₅H₃₁ |
| 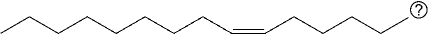 | —C₁₇H₃₅ |
| 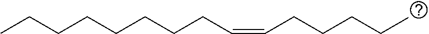 | 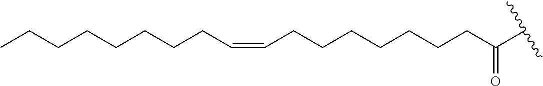 [SMILES: *C(=O)CCCCCCC/C=C\CCCCCCCC] |
| 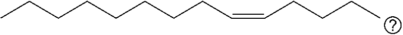 | 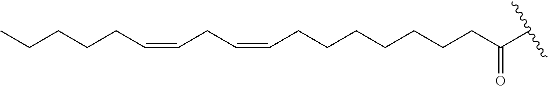 [SMILES: *C(=O)CCCCCCC/C=C\C/C=C\CCCCC] |
| 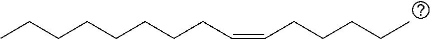 | 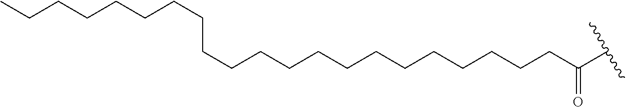 [SMILES: *C(=O)CCCCCCCCCCCCCCCCCCCCC] |
| 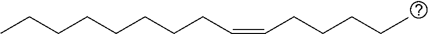 | 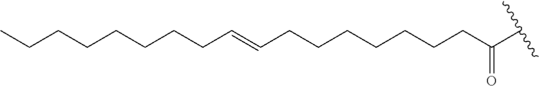 [SMILES: *C(=O)CCCCCCC/C=C/CCCCCCCC] |
| 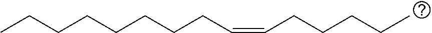 | 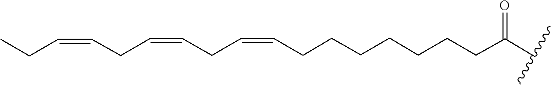 [SMILES: *C(=O)CCCCCCC/C=C\C/C=C\C/C=C\CC] |
| 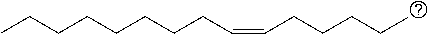 | tBu |
| 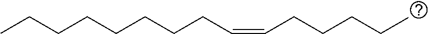 | phenyl |
| 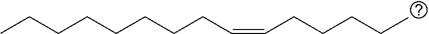 | 4-chlorophenyl |
| 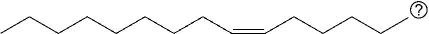 | 3,4-dichlorophenyl |
| 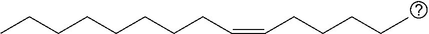 | 4-methylphenyl |
| 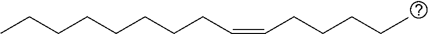 | 4-methoxyphenyl |
| 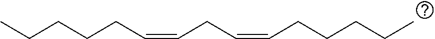 | Me |
| 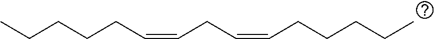 | —C₇H₁₅ |
| 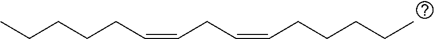 | —C₉H₁₉ |
| 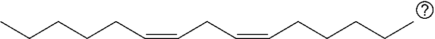 | —C₁₁H₂₃ |
| 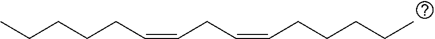 | —C₁₃H₂₇ |
| 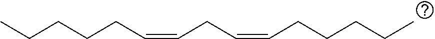 | —C₁₅H₃₁ |
| 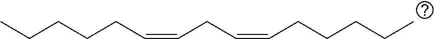 | —C₁₇H₃₅ |
| 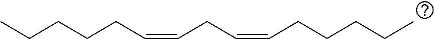 | 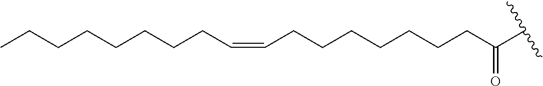 [SMILES: *C(=O)CCCCCCC/C=C\CCCCCCCC] |
| 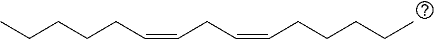 | 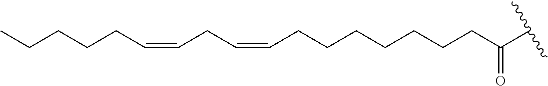 [SMILES: *C(=O)CCCCCCC/C=C\C/C=C\CCCCC] |
| 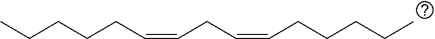 | 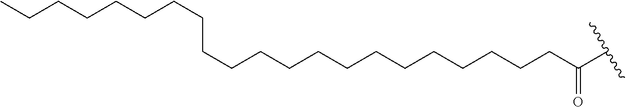 [SMILES: *C(=O)CCCCCCCCCCCCCCCCCCCCC] |
| 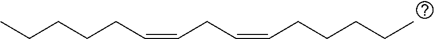 | 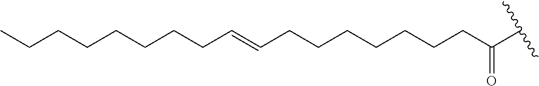 [SMILES: *C(=O)CCCCCCC/C=C/CCCCCCCC] |
| 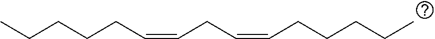 | 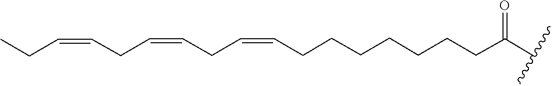 [SMILES: *C(=O)CCCCCCC/C=C\C/C=C\C/C=C\CC] |
| 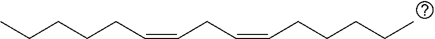 | tBu |
| 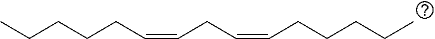 | phenyl |
| 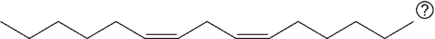 | 4-chlorophenyl |
| 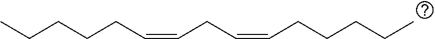 | 3,4-dichlorophenyl |
| 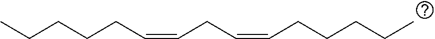 | 4-methylphenyl |
| 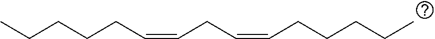 | 4-methoxyphenyl |
| 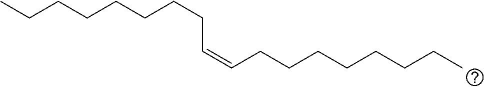 | Me |
| 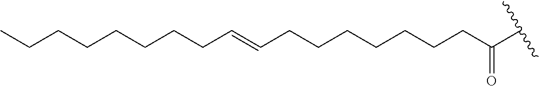 [SMILES: *C(=O)CCCCCCC/C=C/CCCCCCCC] | —C₇H₁₅ |
| 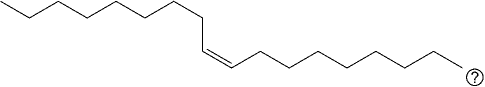 | —C₉H₁₉ |
| 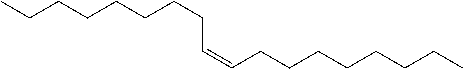 | —C₁₁H₂₃ |
| 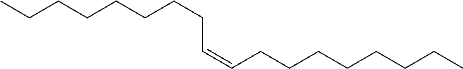 | —C₁₃H₂₇ |
| 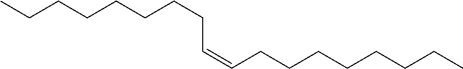 | —C₁₅H₃₁ |
| 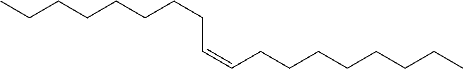 | —C₁₇H₃₅ |
| 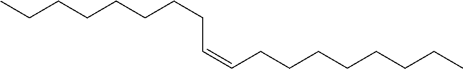 | 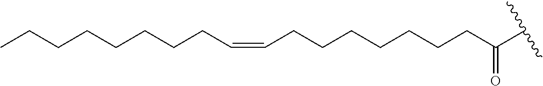 [SMILES: *C(=O)CCCCCCC/C=C\CCCCCCCC] |
| 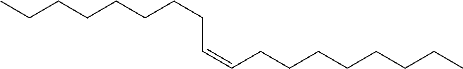 | 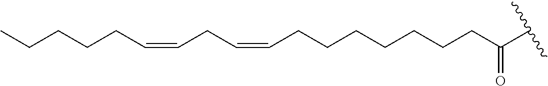 [SMILES: *C(=O)CCCCCCC/C=C\C/C=C\CCCCC] |
| 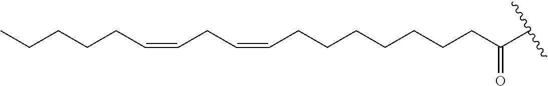 [SMILES: *C(=O)CCCCCCC/C=C\C/C=C\CCCCC] | 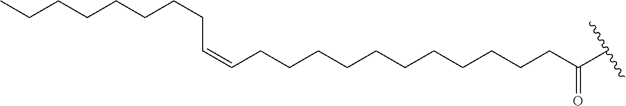 [SMILES: *C(=O)CCCCCCCCCCC/C=C\CCCCCCCC] |
| 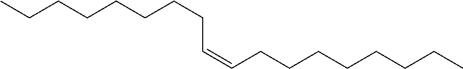 | 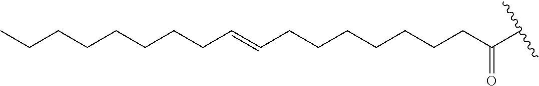 [SMILES: *C(=O)CCCCCCC/C=C/CCCCCCCC] |
| 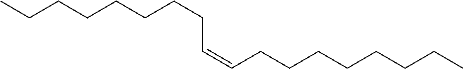 | 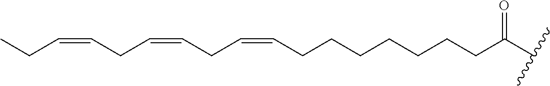 [SMILES: *C(=O)CCCCCCC/C=C\C/C=C\C/C=C\CC] |
| 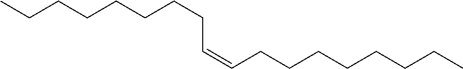 | tBu |
| 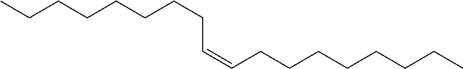 | phenyl |
| 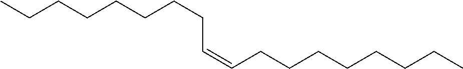 | 4-chlorophenyl |
| 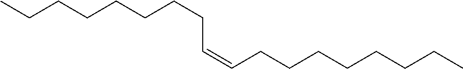 | 3,4-dichlorophenyl |
| 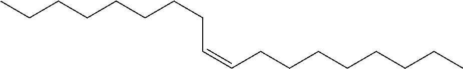 | 4-methylphenyl |
| 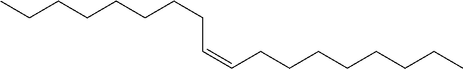 | 4-methoxyphenyl |
| 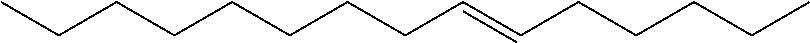 | Me |
| 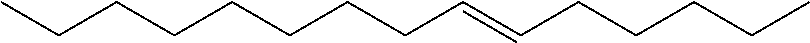 | —C₇H₁₅ |
| 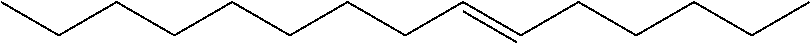 | —C₉H₁₉ |
| 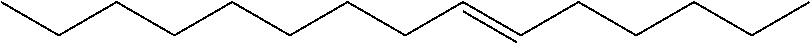 | —C₁₁H₂₃ |
| 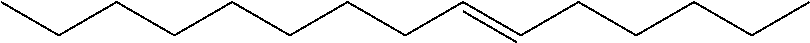 | —C₁₃H₂₇ |
| 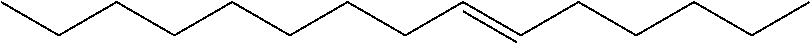 | —C₁₅H₃₁ |
| 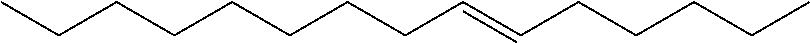 | —C₁₇H₃₅ |
| 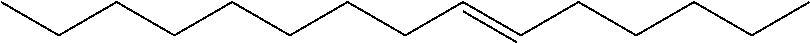 | 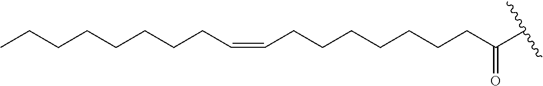 [SMILES: *C(=O)CCCCCCC/C=C\CCCCCCCC] |
| 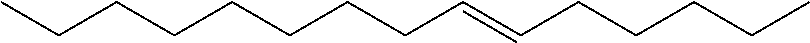 | 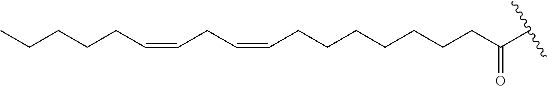 [SMILES: *C(=O)CCCCCCC/C=C\C/C=C\CCCCC] |
| 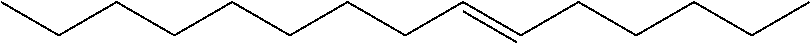 | 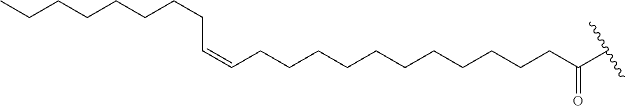 [SMILES: *C(=O)CCCCCCCCCCC/C=C\CCCCCCCC] |
| 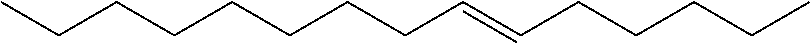 | 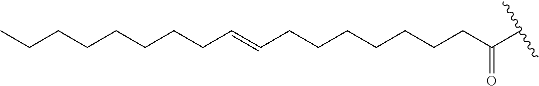 [SMILES: *C(=O)CCCCCCC/C=C/CCCCCCCC] |
| 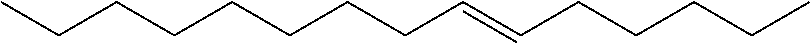 | 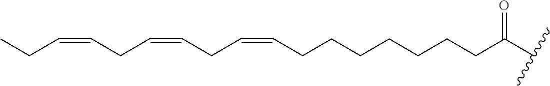 [SMILES: *C(=O)CCCCCCC/C=C\C/C=C\C/C=C\CC] |
| 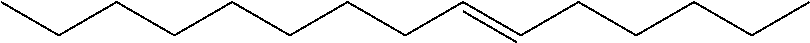 | tBu |
| 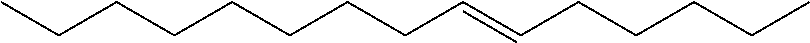 | phenyl |
| 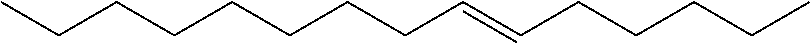 | 4-chlorophenyl |
| 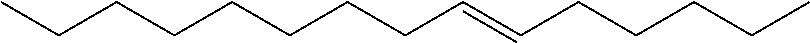 | 3,4-dichlorophenyl |
| 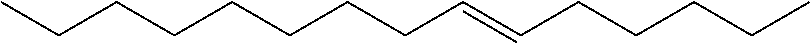 | 4-methylphenyl |
| 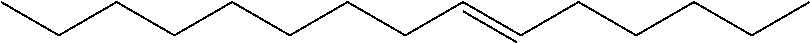 | 4-methoxyphenyl |
| 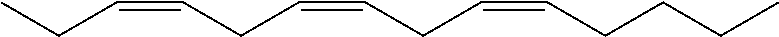 | Me |
| 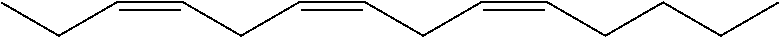 | —C₇H₁₅ |
| 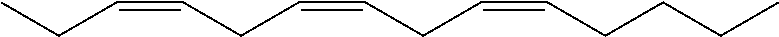 | —C₉H₁₉ |
| 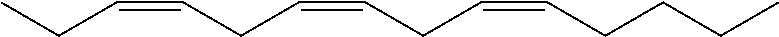 | —C₁₁H₂₃ |
| 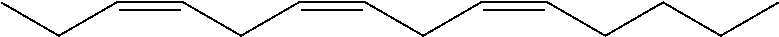 | —C₁₃H₂₇ |
| 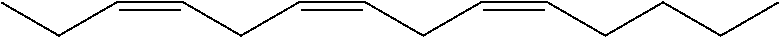 | —C₁₅H₃₁ |
| 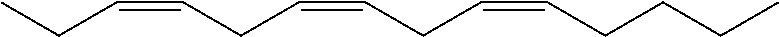 | —C₁₇H₃₅ |
| 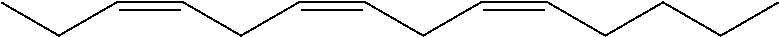 | 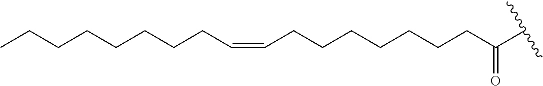 [SMILES: *C(=O)CCCCCCC/C=C\CCCCCCCC] |
| 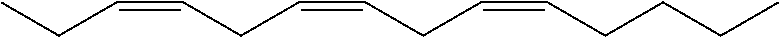 | 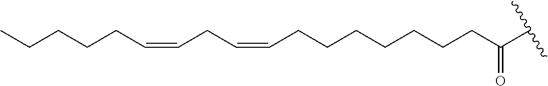 [SMILES: *C(=O)CCCCCCC/C=C\C/C=C\CCCCC] |
| 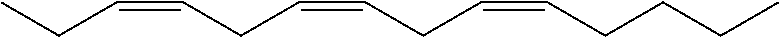 | 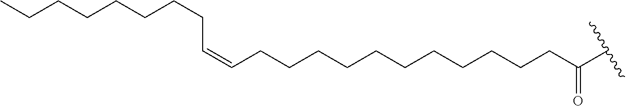 [SMILES: *C(=O)CCCCCCCCCCC/C=C\CCCCCCCC] |
| 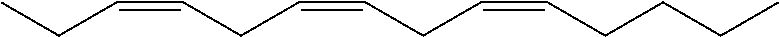 | 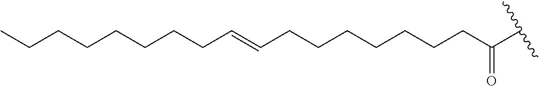 [SMILES: *C(=O)CCCCCCC/C=C/CCCCCCCC] |
| 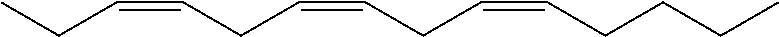 | 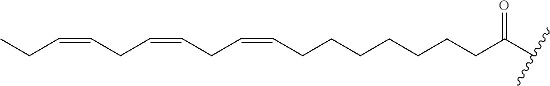 [SMILES: *C(=O)CCCCCCC/C=C\C/C=C\C/C=C\CC] |
| 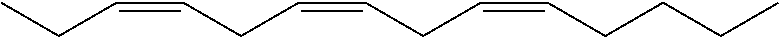 | tBu |
| 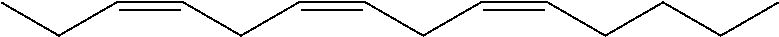 | phenyl |
| 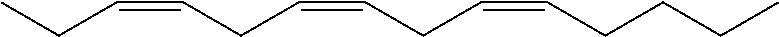 | 4-chlorophenyl |
| 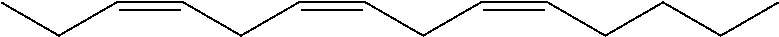 | 3,4-dichlorophenyl |
| 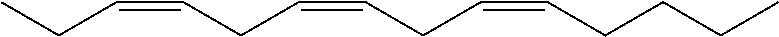 | 4-methylphenyl |
| 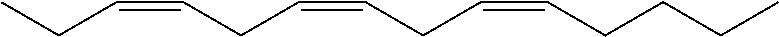 | 4-methoxyphenyl |
| H | 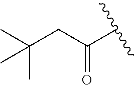 [SMILES: *C(=O)CC(C)(C)C] |
| H | 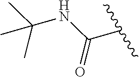 [SMILES: *C(=O)NC(C)(C)C] |
| H | 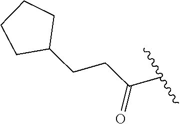 [SMILES: *C(=O)CCC1CCCC1] |
| H | 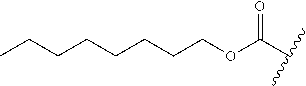 [SMILES: *C(=O)OCCCCCCCC] |
| H | 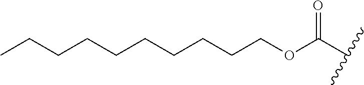 [SMILES: *C(=O)OCCCCCCCCCC] |
| H | 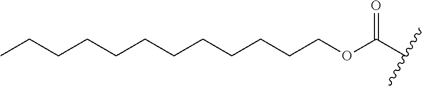 [SMILES: *C(=O)OCCCCCCCCCCCC] |
| H | 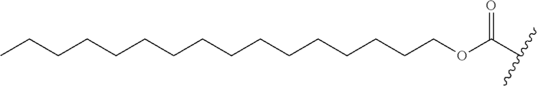 [SMILES: *C(=O)OCCCCCCCCCCCCCCCC] |
| H | 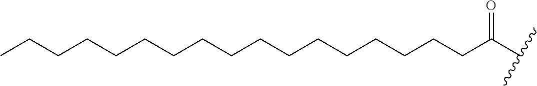 [SMILES: *C(=O)CCCCCCCCCCCCCCCCC] |
| H | 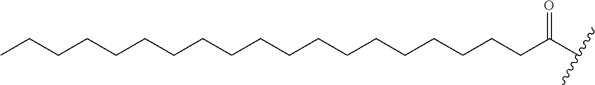 [SMILES: *C(=O)CCCCCCCCCCCCCCCCCCC] |
| H | 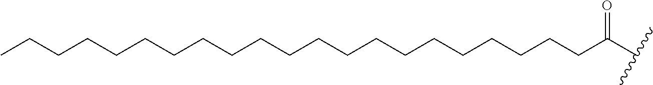 [SMILES: *C(=O)CCCCCCCCCCCCCCCCCCCCC] |
| H | 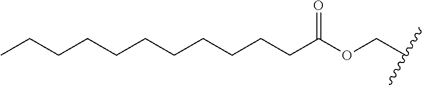 [SMILES: *COC(=O)CCCCCCCCCCC] |
| H | 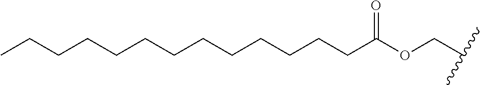 [SMILES: *COC(=O)CCCCCCCCCCCCC] |
| H | 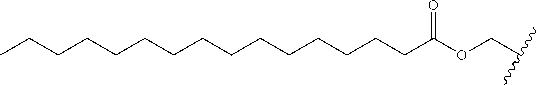 [SMILES: *COC(=O)CCCCCCCCCCCCCCC] |
| H | 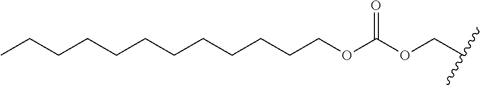 [SMILES: *COC(=O)OCCCCCCCCCCCC] |
| H | 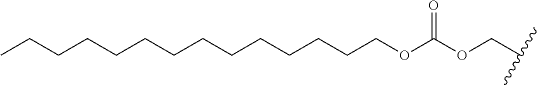 [SMILES: *COC(=O)OCCCCCCCCCCCCCC] |
| H | 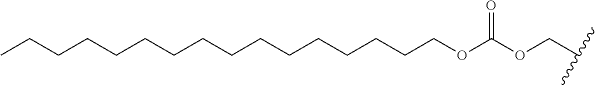 [SMILES: *COC(=O)OCCCCCCCCCCCCCCCC] |
| Me | 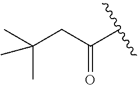 [SMILES: *C(=O)CC(C)(C)C] |
| Me | 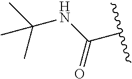 [SMILES: *C(=O)NC(C)(C)C] |
| Me | 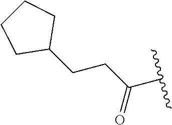 [SMILES: *C(=O)CCC1CCCC1] |
| Me | 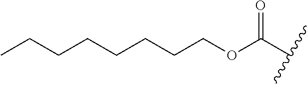 [SMILES: *C(=O)OCCCCCCCC] |
| Me | 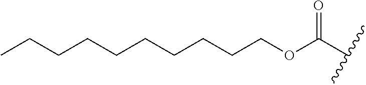 [SMILES: *C(=O)OCCCCCCCCCC] |
| Me | 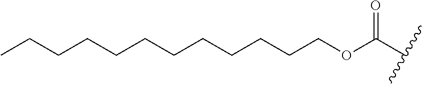 [SMILES: *C(=O)OCCCCCCCCCCCC] |
| Me | 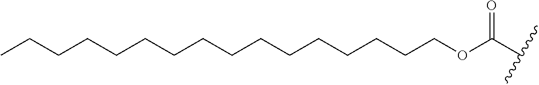 [SMILES: *C(=O)OCCCCCCCCCCCCCCCC] |
| Me | 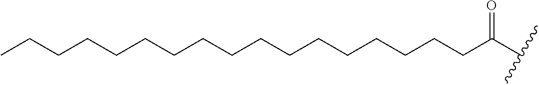 [SMILES: *C(=O)CCCCCCCCCCCCCCCCC] |
| Me | 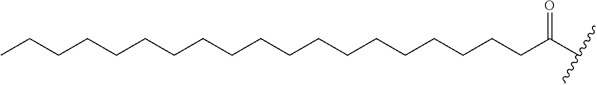 [SMILES: *C(=O)CCCCCCCCCCCCCCCCCCC] |
| Me | 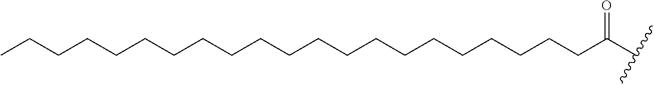 [SMILES: *C(=O)CCCCCCCCCCCCCCCCCCCCC] |
| Me | 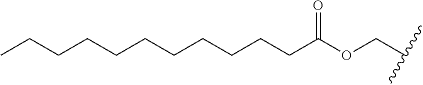 [SMILES: *COC(=O)CCCCCCCCCCC] |
| Me | 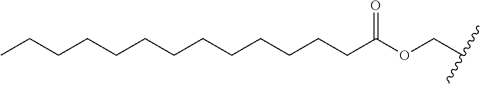 [SMILES: *COC(=O)CCCCCCCCCCCCC] |
| Me | 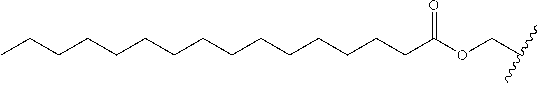 [SMILES: *COC(=O)CCCCCCCCCCCCCCC] |
| Me | 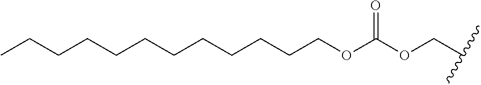 [SMILES: *COC(=O)OCCCCCCCCCCCC] |
| Me | 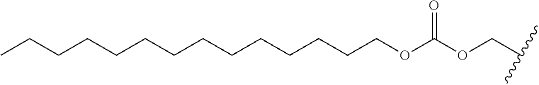 [SMILES: *COC(=O)OCCCCCCCCCCCCCC] |
| Me | 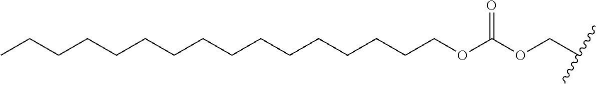 [SMILES: *COC(=O)OCCCCCCCCCCCCCCCC] |
| —C₇H₁₅ | 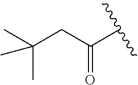 [SMILES: *C(=O)CC(C)(C)C] |
| —C₇H₁₅ | 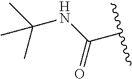 [SMILES: *C(=O)NC(C)(C)C] |
| —C₇H₁₅ | 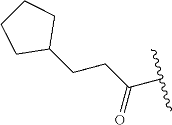 [SMILES: *C(=O)CCC1CCCC1] |
| —C₇H₁₅ | 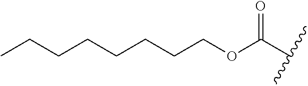 [SMILES: *C(=O)OCCCCCCCC] |
| —C₇H₁₅ | 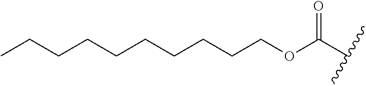 [SMILES: *C(=O)OCCCCCCCCCC] |
| —C₇H₁₅ | 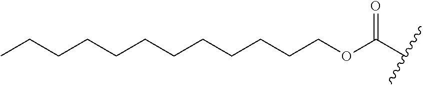 [SMILES: *C(=O)OCCCCCCCCCCCC] |
| —C₇H₁₅ | 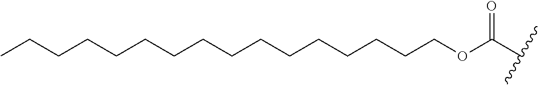 [SMILES: *C(=O)OCCCCCCCCCCCCCCCC] |
| —C₇H₁₅ | 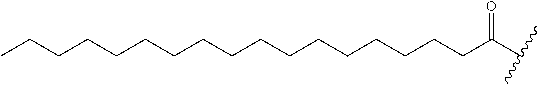 [SMILES: *C(=O)CCCCCCCCCCCCCCCCC] |
| —C₇H₁₅ | 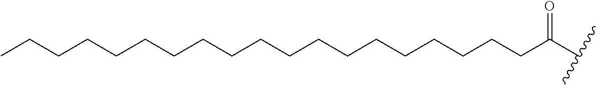 [SMILES: *C(=O)CCCCCCCCCCCCCCCCCCC] |
| —C₇H₁₅ | 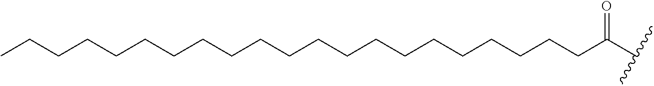 [SMILES: *C(=O)CCCCCCCCCCCCCCCCCCCCC] |
| —C₇H₁₅ | 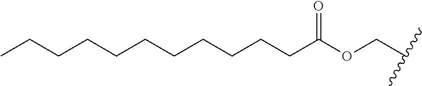 [SMILES: *COC(=O)CCCCCCCCCCC] |
| —C₇H₁₅ | 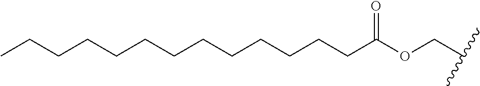 [SMILES: *COC(=O)CCCCCCCCCCCCC] |
| —C₇H₁₅ | 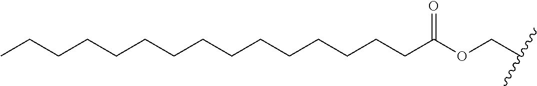 [SMILES: *COC(=O)CCCCCCCCCCCCCCC] |
| —C₇H₁₅ | 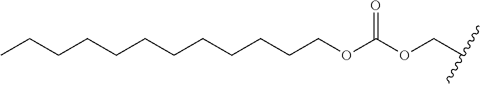 [SMILES: *COC(=O)OCCCCCCCCCCCC] |
| —C₇H₁₅ | 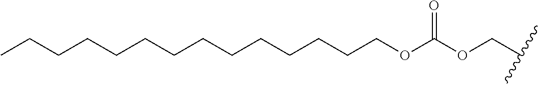 [SMILES: *COC(=O)OCCCCCCCCCCCCCC] |
| —C₇H₁₅ | 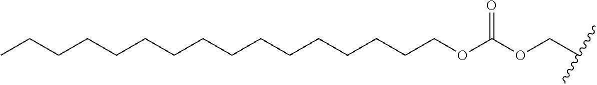 [SMILES: *COC(=O)OCCCCCCCCCCCCCCCC] |
| —C₉H₁₉ | 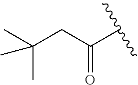 [SMILES: *C(=O)CC(C)(C)C] |
| —C₉H₁₉ | 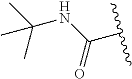 [SMILES: *C(=O)NC(C)(C)C] |
| —C₉H₁₉ | 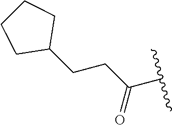 [SMILES: *C(=O)CCC1CCCC1] |
| —C₉H₁₉ | 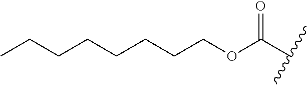 [SMILES: *C(=O)OCCCCCCCC] |
| —C₉H₁₉ | 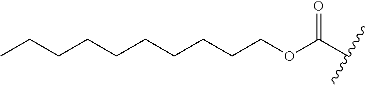 [SMILES: *C(=O)OCCCCCCCCCC] |
| —C₉H₁₉ | 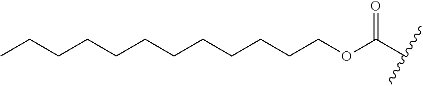 [SMILES: *C(=O)OCCCCCCCCCCCC] |
| —C₉H₁₉ | 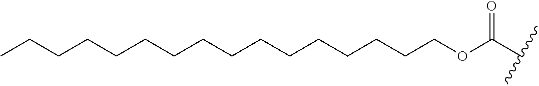 [SMILES: *C(=O)OCCCCCCCCCCCCCCCC] |
| —C₉H₁₉ | 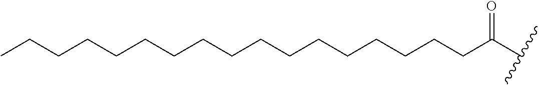 [SMILES: *C(=O)CCCCCCCCCCCCCCCCC] |
| —C₉H₁₉ | 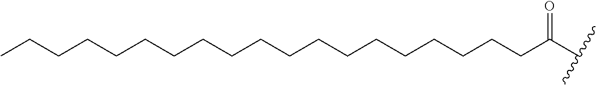 [SMILES: *C(=O)CCCCCCCCCCCCCCCCCCC] |
| —C₉H₁₉ | 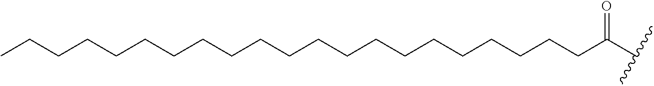 [SMILES: *C(=O)CCCCCCCCCCCCCCCCCCCCC] |
| —C₉H₁₉ | 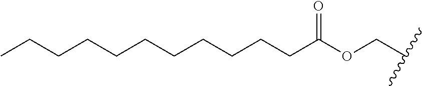 [SMILES: *COC(=O)CCCCCCCCCCC] |
| —C₉H₁₉ | 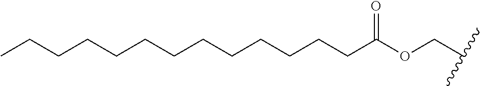 [SMILES: *COC(=O)CCCCCCCCCCCCC] |
| —C₉H₁₉ | 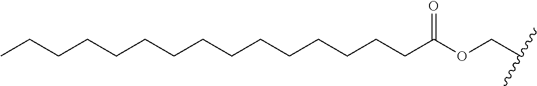 [SMILES: *COC(=O)CCCCCCCCCCCCCCC] |
| —C₉H₁₉ | 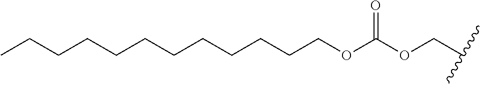 [SMILES: *COC(=O)OCCCCCCCCCCCC] |
| —C₉H₁₉ | 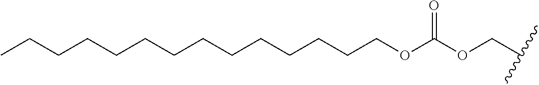 [SMILES: *COC(=O)OCCCCCCCCCCCCCC] |
| —C₉H₁₉ | 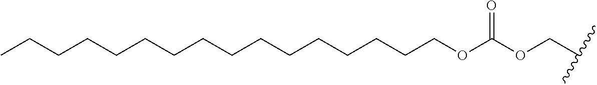 [SMILES: *COC(=O)OCCCCCCCCCCCCCCCC] |
| —C₁₁H₂₃ | 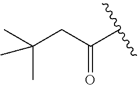 [SMILES: *C(=O)CC(C)(C)C] |
| —C₁₁H₂₃ | 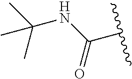 [SMILES: *C(=O)NC(C)(C)C] |
| —C₁₁H₂₃ | 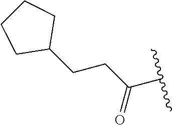 [SMILES: *C(=O)CCC1CCCC1] |
| —C₁₁H₂₃ | 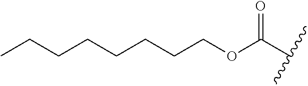 [SMILES: *C(=O)OCCCCCCCC] |
| —C₁₁H₂₃ | 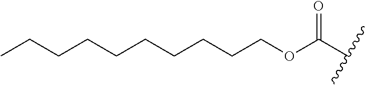 [SMILES: *C(=O)OCCCCCCCCCC] |
| —C₁₁H₂₃ | 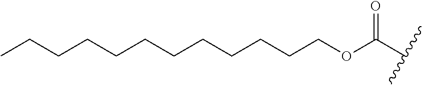 [SMILES: *C(=O)OCCCCCCCCCCCC] |
| —C₁₁H₂₃ | 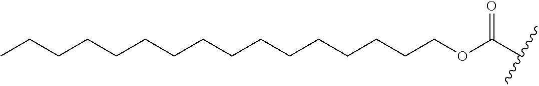 [SMILES: *C(=O)OCCCCCCCCCCCCCCCC] |
| —C₁₁H₂₃ | 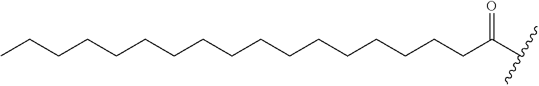 [SMILES: *C(=O)CCCCCCCCCCCCCCCCC] |
| —C₁₁H₂₃ | 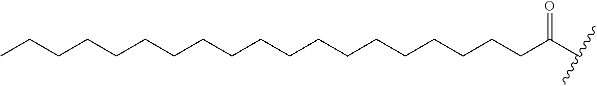 [SMILES: *C(=O)CCCCCCCCCCCCCCCCCCC] |
| —C₁₁H₂₃ | 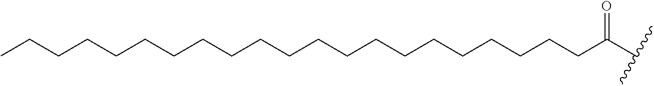 [SMILES: *C(=O)CCCCCCCCCCCCCCCCCCCCC] |
| —C₁₁H₂₃ | 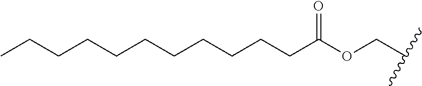 [SMILES: *COC(=O)CCCCCCCCCCC] |
| —C₁₁H₂₃ | 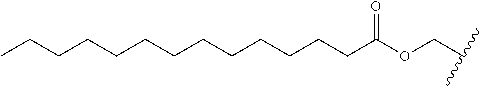 [SMILES: *COC(=O)CCCCCCCCCCCCC] |
| —C₁₁H₂₃ | 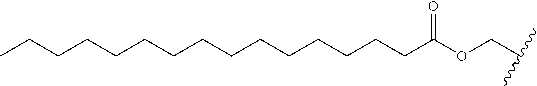 [SMILES: *COC(=O)CCCCCCCCCCCCCCC] |
| —C₁₁H₂₃ | 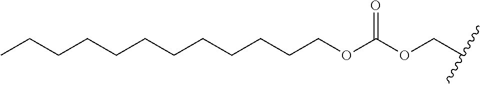 [SMILES: *COC(=O)OCCCCCCCCCCCC] |
| —C₁₁H₂₃ | 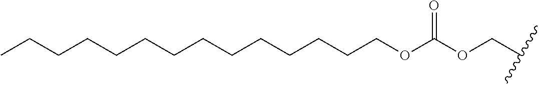 [SMILES: *COC(=O)OCCCCCCCCCCCCCC] |
| —C₁₁H₂₃ | 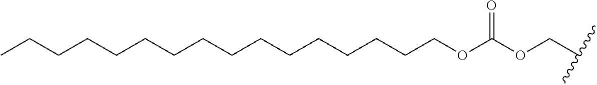 [SMILES: *COC(=O)OCCCCCCCCCCCCCCCC] |
| —C₁₃H₂₇ | 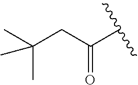 [SMILES: *C(=O)CC(C)(C)C] |
| —C₁₃H₂₇ | 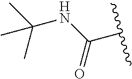 [SMILES: *C(=O)NC(C)(C)C] |
| —C₁₃H₂₇ | 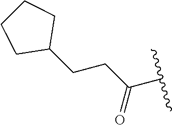 [SMILES: *C(=O)CCC1CCCC1] |
| —C₁₃H₂₇ | 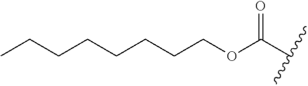 [SMILES: *C(=O)OCCCCCCCC] |
| —C₁₃H₂₇ | 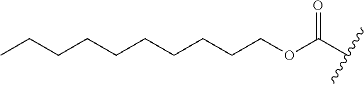 [SMILES: *C(=O)OCCCCCCCCCC] |
| —C₁₃H₂₇ | 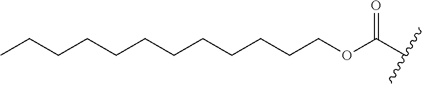 [SMILES: *C(=O)OCCCCCCCCCCCC] |
| —C₁₃H₂₇ | 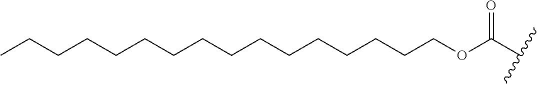 [SMILES: *C(=O)OCCCCCCCCCCCCCCCC] |
| —C₁₃H₂₇ | 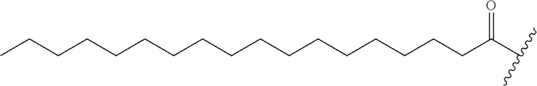 [SMILES: *C(=O)CCCCCCCCCCCCCCCCC] |
| —C₁₃H₂₇ | 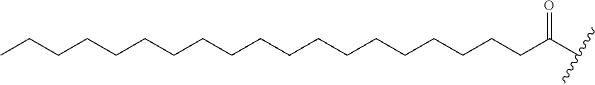 [SMILES: *C(=O)CCCCCCCCCCCCCCCCCCC] |
| —C₁₃H₂₇ | 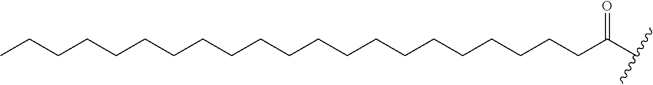 [SMILES: *C(=O)CCCCCCCCCCCCCCCCCCCCC] |
| —C₁₃H₂₇ | 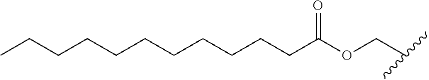 [SMILES: *COC(=O)CCCCCCCCCCC] |
| —C₁₃H₂₇ | 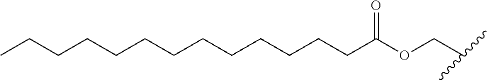 [SMILES: *COC(=O)CCCCCCCCCCCCC] |
| —C₁₃H₂₇ | 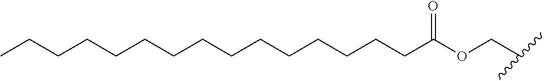 [SMILES: *COC(=O)CCCCCCCCCCCCCCC] |
| —C₁₃H₂₇ | 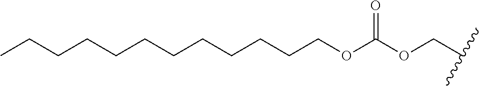 [SMILES: *COC(=O)OCCCCCCCCCCCC] |
| —C₁₃H₂₇ | 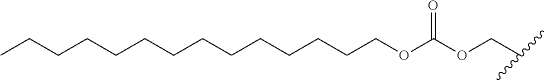 [SMILES: *COC(=O)OCCCCCCCCCCCCCC] |
| —C₁₃H₂₇ | 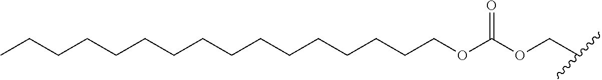 [SMILES: *COC(=O)OCCCCCCCCCCCCCCCC] |
| —C₁₅H₃₁ | 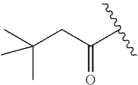 [SMILES: *C(=O)CC(C)(C)C] |
| —C₁₅H₃₁ | 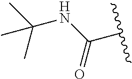 [SMILES: *C(=O)NC(C)(C)C] |
| —C₁₅H₃₁ | 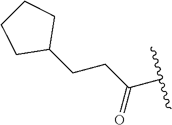 [SMILES: *C(=O)CCC1CCCC1] |
| —C₁₅H₃₁ | 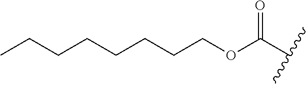 [SMILES: *C(=O)OCCCCCCCC] |
| —C₁₅H₃₁ | 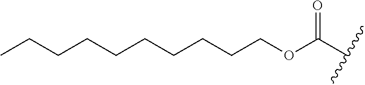 [SMILES: *C(=O)OCCCCCCCCCC] |
| —C₁₅H₃₁ | 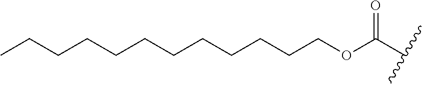 [SMILES: *C(=O)OCCCCCCCCCCCC] |
| —C₁₅H₃₁ | 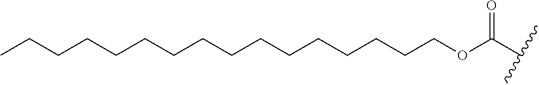 [SMILES: *C(=O)OCCCCCCCCCCCCCCCC] |
| —C₁₅H₃₁ | 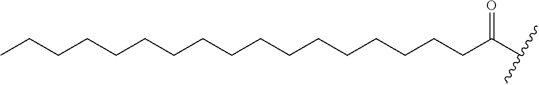 [SMILES: *C(=O)CCCCCCCCCCCCCCCCC] |
| —C₁₅H₃₁ | 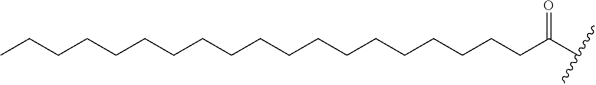 [SMILES: *C(=O)CCCCCCCCCCCCCCCCCCC] |
| —C₁₅H₃₁ | 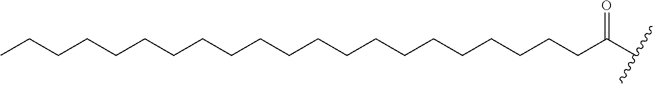 [SMILES: *C(=O)CCCCCCCCCCCCCCCCCCCCC] |
| —C₁₅H₃₁ | 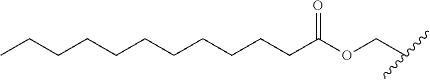 [SMILES: *COC(=O)CCCCCCCCCCC] |
| —C₁₅H₃₁ | 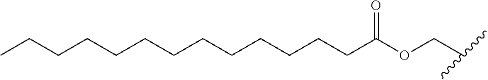 [SMILES: *COC(=O)CCCCCCCCCCCCC] |
| —C₁₅H₃₁ | 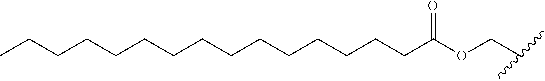 [SMILES: *COC(=O)CCCCCCCCCCCCCCC] |
| —C₁₅H₃₁ | 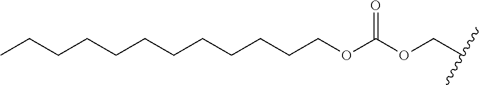 [SMILES: *COC(=O)OCCCCCCCCCCCC] |
| —C₁₅H₃₁ | 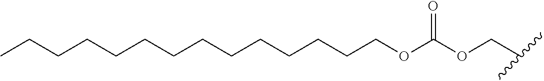 [SMILES: *COC(=O)OCCCCCCCCCCCCCC] |
| —C₁₅H₃₁ | 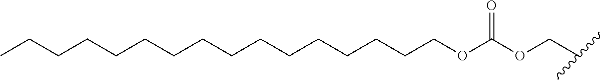 [SMILES: *COC(=O)OCCCCCCCCCCCCCCCC] |
| —C₁₇H₃₅ | 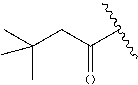 [SMILES: *C(=O)CC(C)(C)C] |
| —C₁₇H₃₅ | 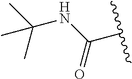 [SMILES: *C(=O)NC(C)(C)C] |
| —C₁₇H₃₅ | 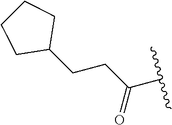 [SMILES: *C(=O)CCC1CCCC1] |
| —C₁₇H₃₅ | 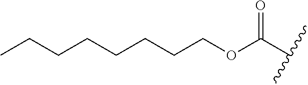 [SMILES: *C(=O)OCCCCCCCC] |
| —C₁₇H₃₅ | 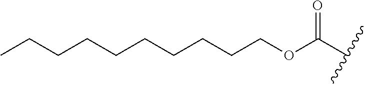 [SMILES: *C(=O)OCCCCCCCCCC] |
| —C₁₇H₃₅ | 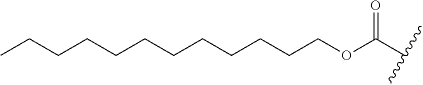 [SMILES: *C(=O)OCCCCCCCCCCCC] |
| —C₁₇H₃₅ | 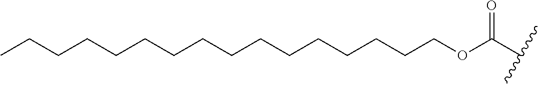 [SMILES: *C(=O)OCCCCCCCCCCCCCCCC] |
| —C₁₇H₃₅ | 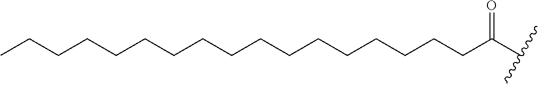 [SMILES: *C(=O)CCCCCCCCCCCCCCCCC] |
| —C₁₇H₃₅ | 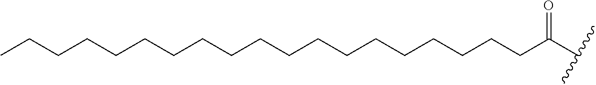 [SMILES: *C(=O)CCCCCCCCCCCCCCCCCCC] |
| —C₁₇H₃₅ | 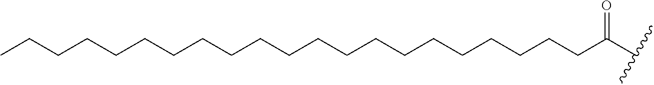 [SMILES: *C(=O)CCCCCCCCCCCCCCCCCCCCC] |
| —C₁₇H₃₅ | 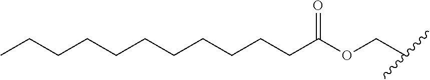 [SMILES: *COC(=O)CCCCCCCCCCC] |
| —C₁₇H₃₅ | 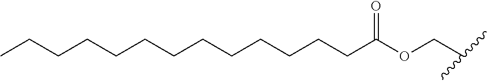 [SMILES: *COC(=O)CCCCCCCCCCCCC] |
| —C₁₇H₃₅ | 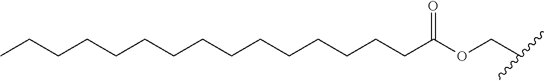 [SMILES: *COC(=O)CCCCCCCCCCCCCCC] |
| —C₁₇H₃₅ | 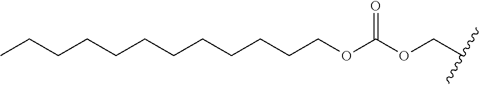 [SMILES: *COC(=O)OCCCCCCCCCCCC] |
| —C₁₇H₃₅ | 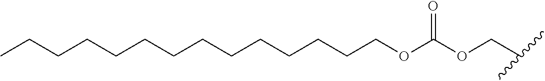 [SMILES: *COC(=O)OCCCCCCCCCCCCCC] |
| —C₁₇H₃₅ | 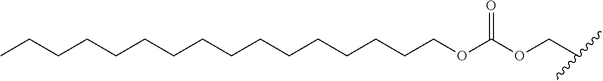 [SMILES: *COC(=O)OCCCCCCCCCCCCCCCC] |
| 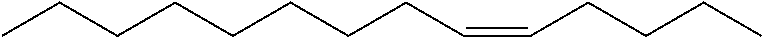 | 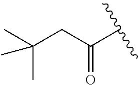 [SMILES: *C(=O)CC(C)(C)C] |
| 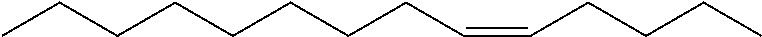 | 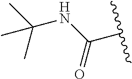 [SMILES: *C(=O)NC(C)(C)C] |
| 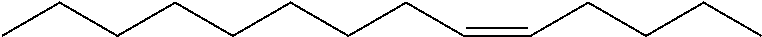 | 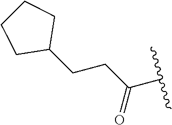 [SMILES: *C(=O)CCC1CCCC1] |
| 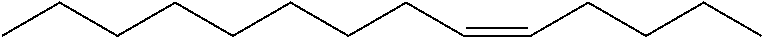 | 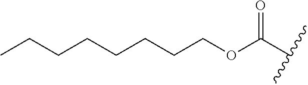 [SMILES: *C(=O)OCCCCCCCC] |
| 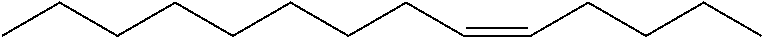 | 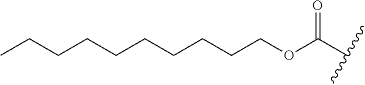 [SMILES: *C(=O)OCCCCCCCCCC] |
| 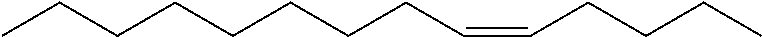 | 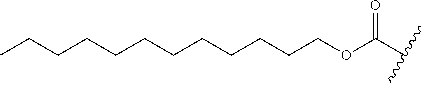 [SMILES: *C(=O)OCCCCCCCCCCCC] |
| 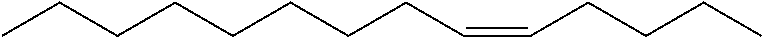 | 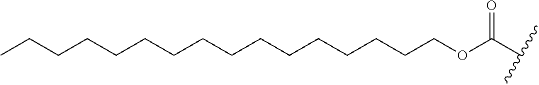 [SMILES: *C(=O)OCCCCCCCCCCCCCCCC] |
| 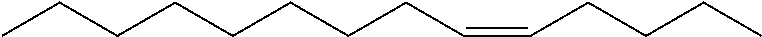 | 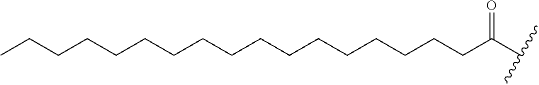 [SMILES: *C(=O)CCCCCCCCCCCCCCCCC] |
| 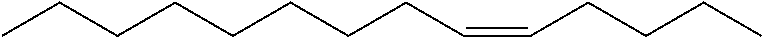 | 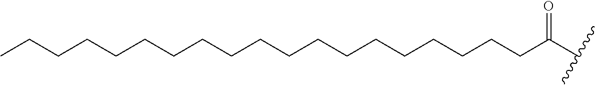 [SMILES: *C(=O)CCCCCCCCCCCCCCCCCCC] |
| 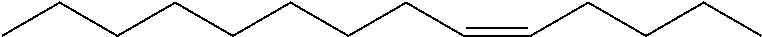 | 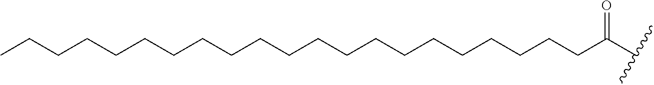 [SMILES: *C(=O)CCCCCCCCCCCCCCCCCCCCC] |
| 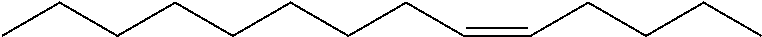 | 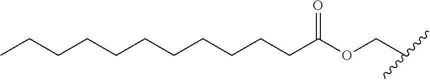 [SMILES: *COC(=O)CCCCCCCCCCC] |
| 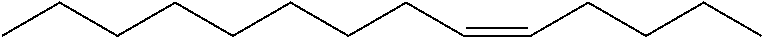 | 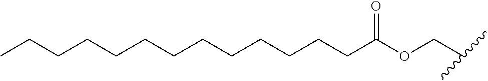 [SMILES: *COC(=O)CCCCCCCCCCCCC] |
| 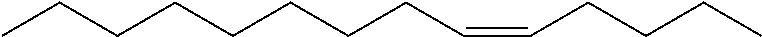 | 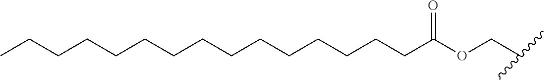 [SMILES: *COC(=O)CCCCCCCCCCCCCCC] |
| 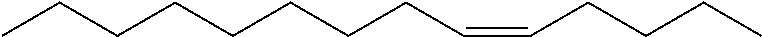 | 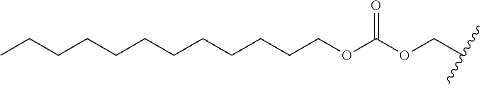 [SMILES: *COC(=O)OCCCCCCCCCCCC] |
| 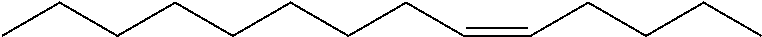 | 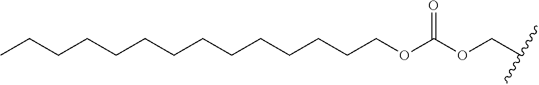 [SMILES: *COC(=O)OCCCCCCCCCCCCCC] |
| 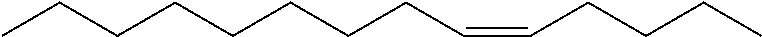 | 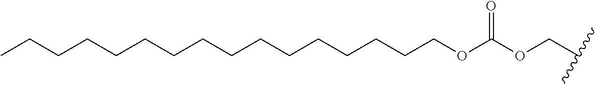 [SMILES: *COC(=O)OCCCCCCCCCCCCCCCC] |
| 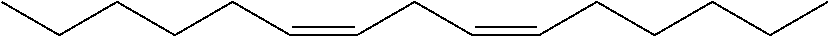 | 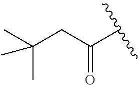 [SMILES: *C(=O)CC(C)(C)C] |
| 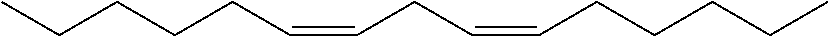 | 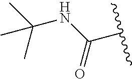 [SMILES: *C(=O)NC(C)(C)C] |
| 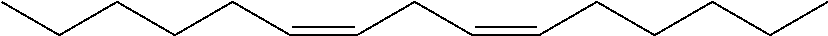 | 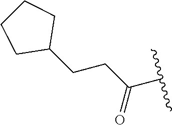 [SMILES: *C(=O)CCC1CCCC1] |
| 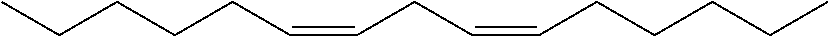 | 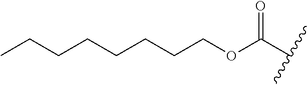 [SMILES: *C(=O)OCCCCCCCC] |
| 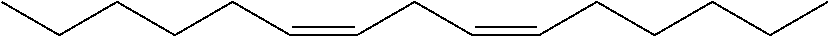 | 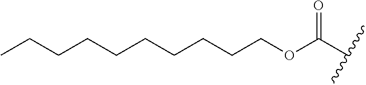 [SMILES: *C(=O)OCCCCCCCCCC] |
| 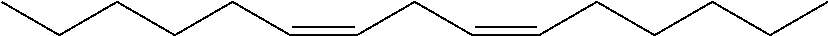 | 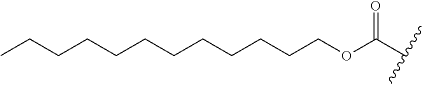 [SMILES: *C(=O)OCCCCCCCCCCCC] |
| 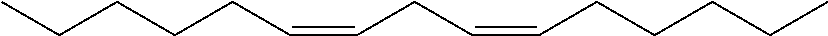 | 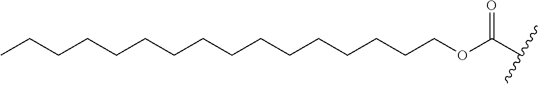 [SMILES: *C(=O)OCCCCCCCCCCCCCCCC] |
| 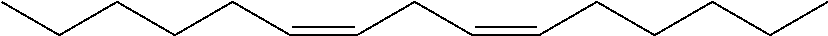 | 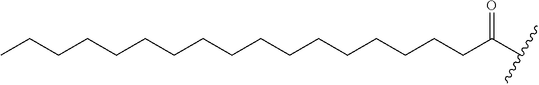 [SMILES: *C(=O)CCCCCCCCCCCCCCCCC] |
| 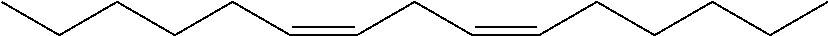 | 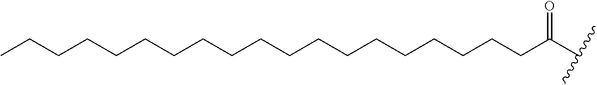 [SMILES: *C(=O)CCCCCCCCCCCCCCCCCCC] |
| 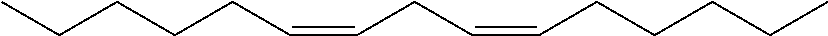 | 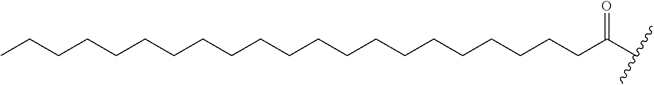 [SMILES: *C(=O)CCCCCCCCCCCCCCCCCCCCC] |
| 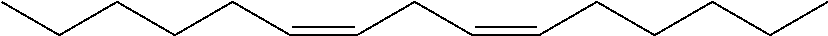 | 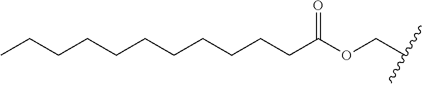 [SMILES: *COC(=O)CCCCCCCCCCC] |
| 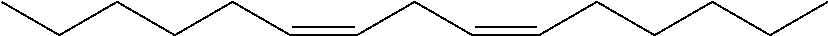 | 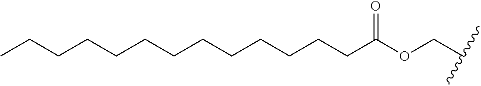 [SMILES: *COC(=O)CCCCCCCCCCCCC] |
| 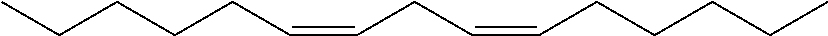 | 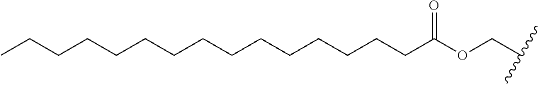 [SMILES: *COC(=O)CCCCCCCCCCCCCCC] |
| 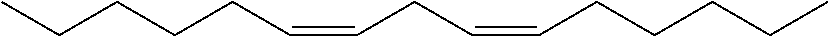 | 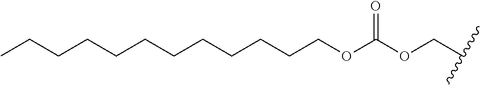 [SMILES: *COC(=O)OCCCCCCCCCCCC] |
| 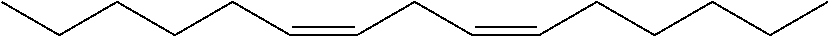 | 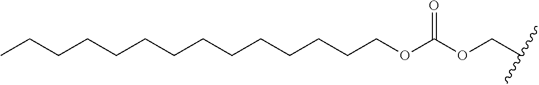 [SMILES: *COC(=O)OCCCCCCCCCCCCCC] |
| 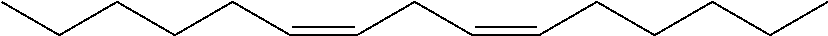 | 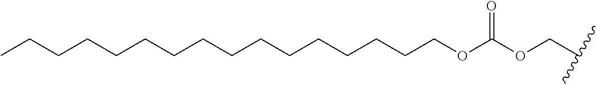 [SMILES: *COC(=O)OCCCCCCCCCCCCCCCC] |
| 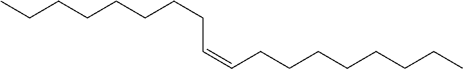 | 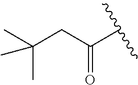 [SMILES: *C(=O)CC(C)(C)C] |
| 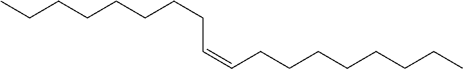 | 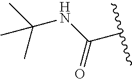 [SMILES: *C(=O)NC(C)(C)C] |
| 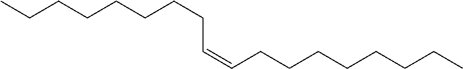 | 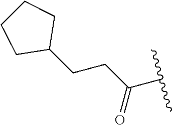 [SMILES: *C(=O)CCC1CCCC1] |
| 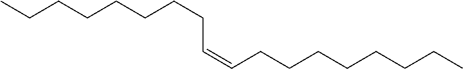 | 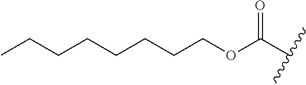 [SMILES: *C(=O)OCCCCCCCC] |
| 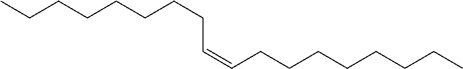 | 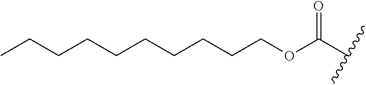 [SMILES: *C(=O)OCCCCCCCCCC] |
| 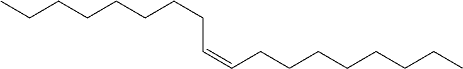 | 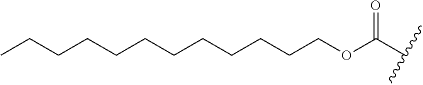 [SMILES: *C(=O)OCCCCCCCCCCCC] |
| 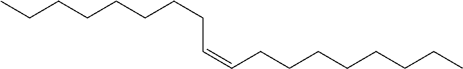 | 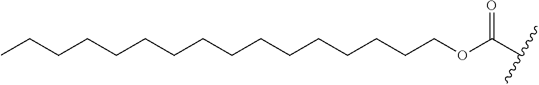 [SMILES: *C(=O)OCCCCCCCCCCCCCCCC] |
| 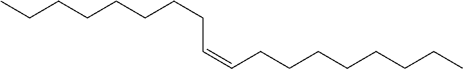 | 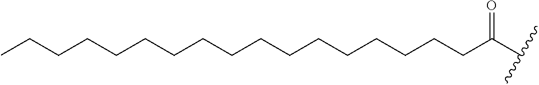 [SMILES: *C(=O)CCCCCCCCCCCCCCCCC] |
| 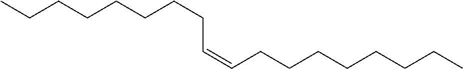 | 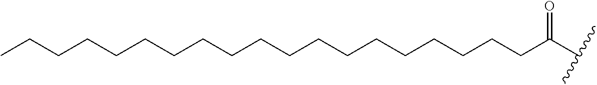 [SMILES: *C(=O)CCCCCCCCCCCCCCCCCCC] |
| 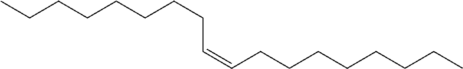 | 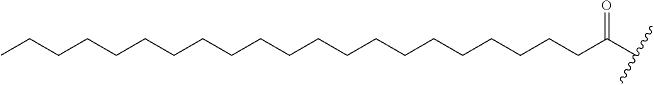 [SMILES: *C(=O)CCCCCCCCCCCCCCCCCCCCC] |
| 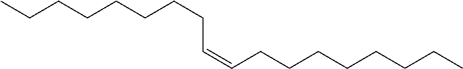 | 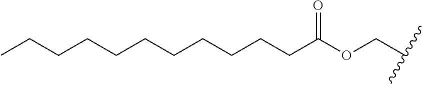 [SMILES: *COC(=O)CCCCCCCCCCC] |
| 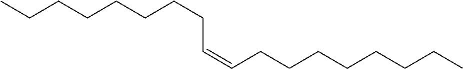 | 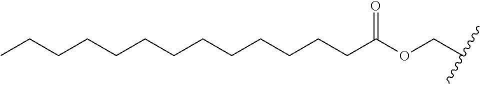 [SMILES: *COC(=O)CCCCCCCCCCCCC] |
| 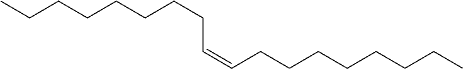 | 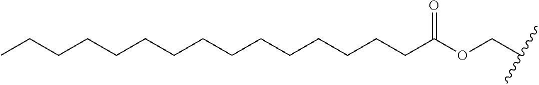 [SMILES: *COC(=O)CCCCCCCCCCCCCCC] |
| 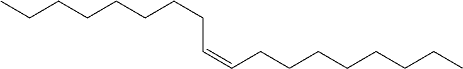 | 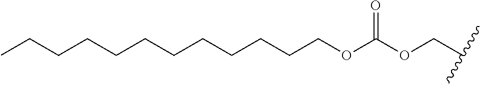 [SMILES: *COC(=O)OCCCCCCCCCCCC] |
| 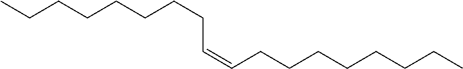 | 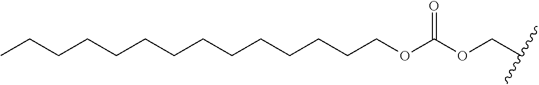 [SMILES: *COC(=O)OCCCCCCCCCCCCCC] |
| 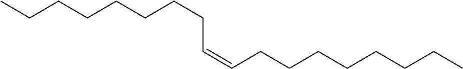 | 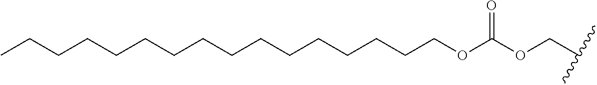 [SMILES: *COC(=O)OCCCCCCCCCCCCCCCC] |
| 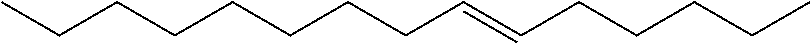 | 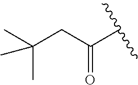 [SMILES: *C(=O)CC(C)(C)C] |
| 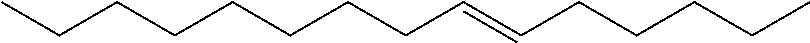 | 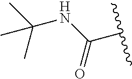 [SMILES: *C(=O)NC(C)(C)C] |
| 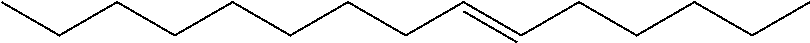 | 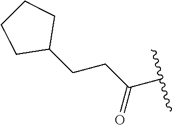 [SMILES: *C(=O)CCC1CCCC1] |
| 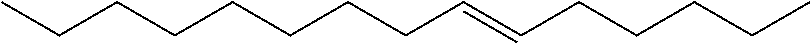 | 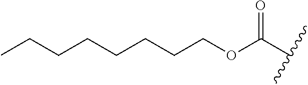 [SMILES: *C(=O)OCCCCCCCC] |
| 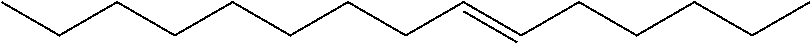 | 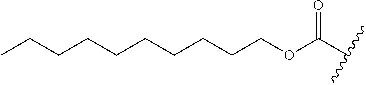 [SMILES: *C(=O)OCCCCCCCCCC] |
| 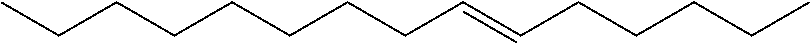 | 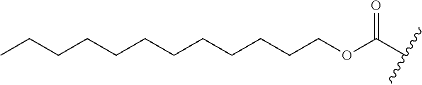 [SMILES: *C(=O)OCCCCCCCCCCCC] |
| 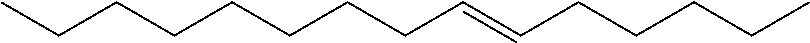 | 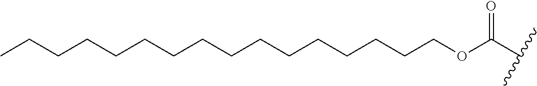 [SMILES: *C(=O)OCCCCCCCCCCCCCCCC] |
| 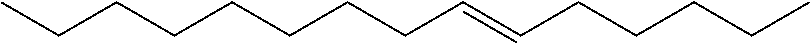 | 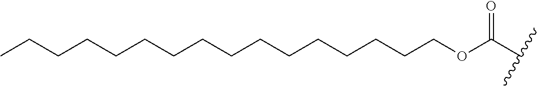 [SMILES: *C(=O)OCCCCCCCCCCCCCCCC] |
| 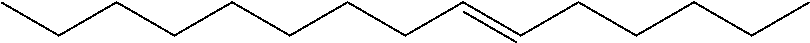 | 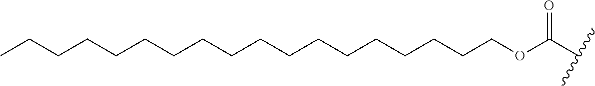 [SMILES: *C(=O)OCCCCCCCCCCCCCCCCCC] |
| 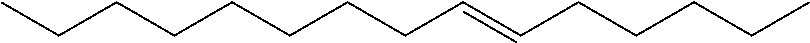 | 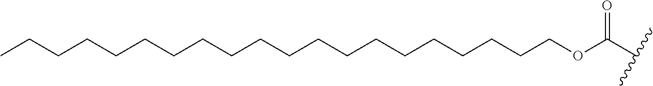 [SMILES: *C(=O)OCCCCCCCCCCCCCCCCCCCC] |
| 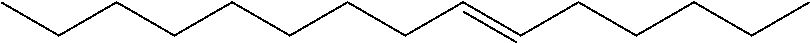 | 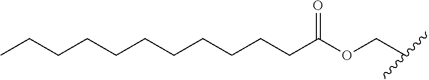 [SMILES: *COC(=O)CCCCCCCCCCC] |
| 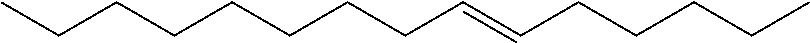 | 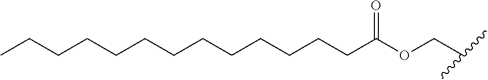 [SMILES: *COC(=O)CCCCCCCCCCCCC] |
| 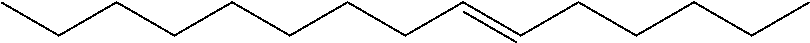 | 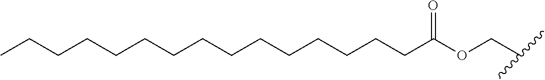 [SMILES: *COC(=O)CCCCCCCCCCCCCCC] |
| 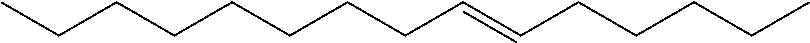 | 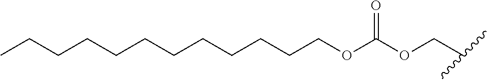 [SMILES: *COC(=O)OCCCCCCCCCCCC] |
| 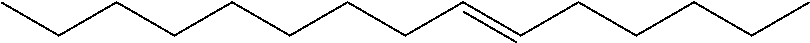 | 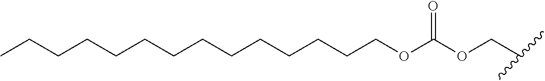 [SMILES: *COC(=O)OCCCCCCCCCCCCCC] |
| 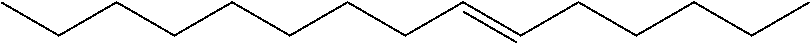 | 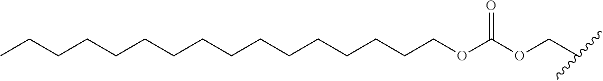 [SMILES: *COC(=O)OCCCCCCCCCCCCCCCC] |
| 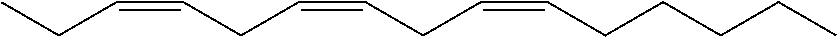 | 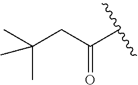 [SMILES: *C(=O)CC(C)(C)C] |
| 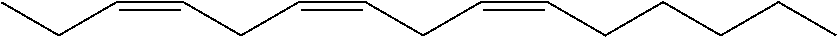 | 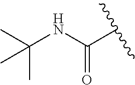 [SMILES: *C(=O)NC(C)(C)C] |
| 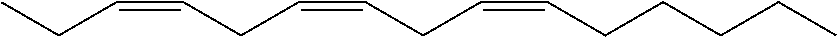 | 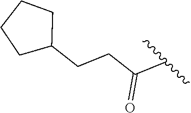 [SMILES: *C(=O)CCC1CCCC1] |
| 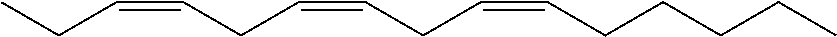 | 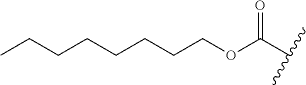 [SMILES: *C(=O)OCCCCCCCC] |
| 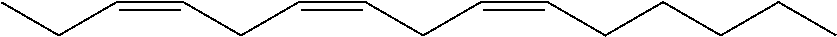 | 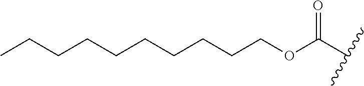 [SMILES: *C(=O)OCCCCCCCCCC] |
| 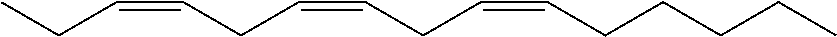 | 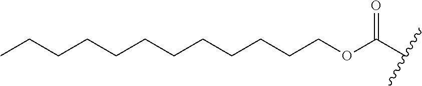 [SMILES: *C(=O)OCCCCCCCCCCCC] |
| 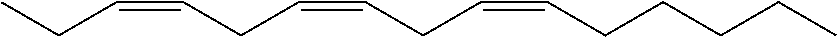 | 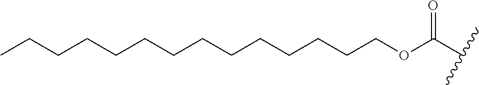 [SMILES: *C(=O)OCCCCCCCCCCCCCC] |
| 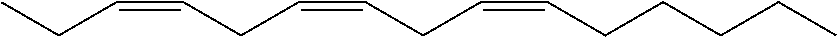 | 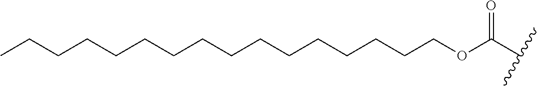 [SMILES: *C(=O)OCCCCCCCCCCCCCCCC] |
| 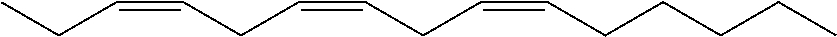 | 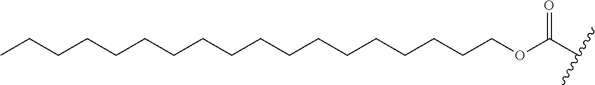 [SMILES: *C(=O)OCCCCCCCCCCCCCCCCCC] |
| 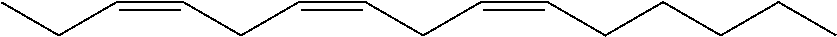 | 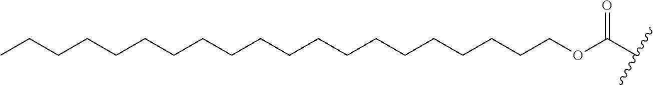 [SMILES: *C(=O)OCCCCCCCCCCCCCCCCCCCC] |
| 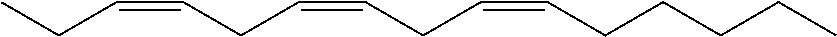 | 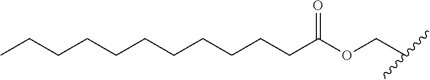 [SMILES: *COC(=O)CCCCCCCCCCC] |
| 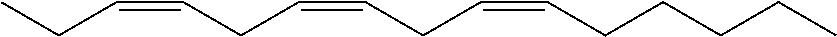 | 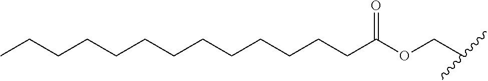 [SMILES: *COC(=O)CCCCCCCCCCCCC] |
| 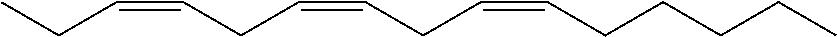 | 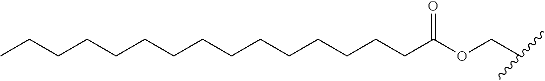 [SMILES: *COC(=O)CCCCCCCCCCCCCCC] |
| 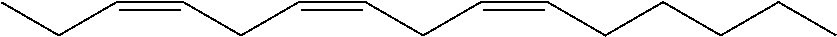 | 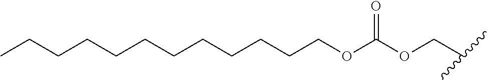 [SMILES: *COC(=O)OCCCCCCCCCCCC] |
| 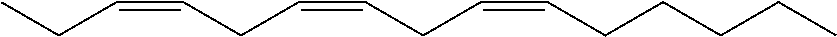 | 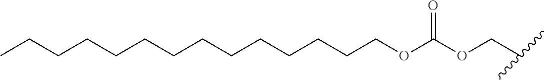 [SMILES: *COC(=O)OCCCCCCCCCCCCCC] |
| 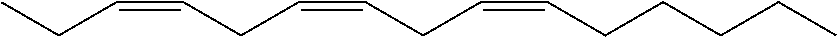 | 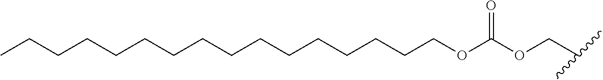 [SMILES: *COC(=O)OCCCCCCCCCCCCCCCC] |
| tBu | 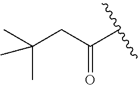 [SMILES: *C(=O)CC(C)(C)C] |
| tBu | 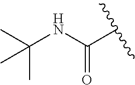 [SMILES: *C(=O)NC(C)(C)C] |
| tBu | 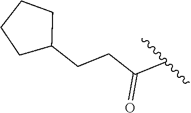 [SMILES: *C(=O)CCC1CCCC1] |
| tBu | 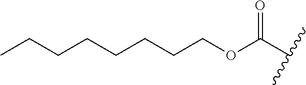 [SMILES: *C(=O)OCCCCCCCC] |
| tBu | 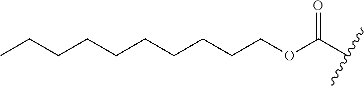 [SMILES: *C(=O)OCCCCCCCCCC] |
| tBu | 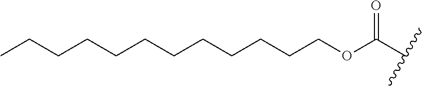 [SMILES: *C(=O)OCCCCCCCCCCCC] |
| tBu | 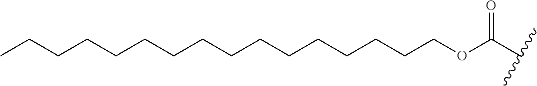 [SMILES: *C(=O)OCCCCCCCCCCCCCCCC] |
| tBu | 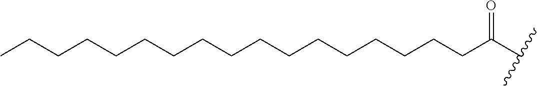 [SMILES: *C(=O)CCCCCCCCCCCCCCCCC] |
| tBu | 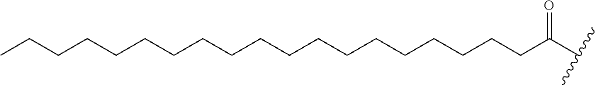 [SMILES: *C(=O)CCCCCCCCCCCCCCCCCCC] |
| tBu | 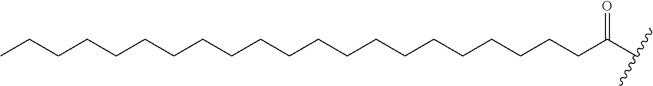 [SMILES: *C(=O)CCCCCCCCCCCCCCCCCCCCC] |
| tBu | 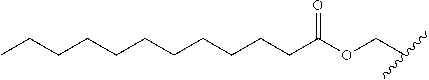 [SMILES: *COC(=O)CCCCCCCCCCC] |
| tBu | 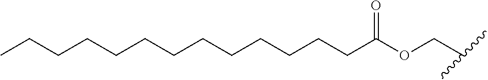 [SMILES: *COC(=O)CCCCCCCCCCCCC] |
| tBu | 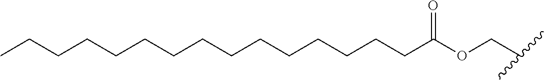 [SMILES: *COC(=O)CCCCCCCCCCCCCCC] |
| tBu | 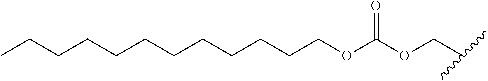 [SMILES: *COC(=O)OCCCCCCCCCCCC] |
| tBu | 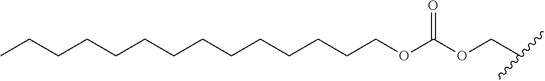 [SMILES: *COC(=O)OCCCCCCCCCCCCCC] |
| tBu | 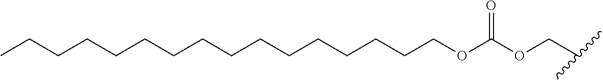 [SMILES: *COC(=O)OCCCCCCCCCCCCCCCC] |
| phenyl | 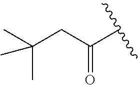 [SMILES: *C(=O)CC(C)(C)C] |
| phenyl | 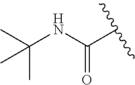 [SMILES: *C(=O)NC(C)(C)C] |
| phenyl | 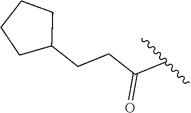 [SMILES: *C(=O)CCC1CCCC1] |
| phenyl | 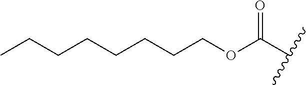 [SMILES: *C(=O)OCCCCCCCC] |
| phenyl | 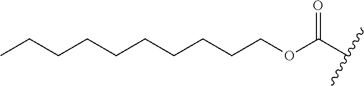 [SMILES: *C(=O)OCCCCCCCCCC] |
| phenyl | 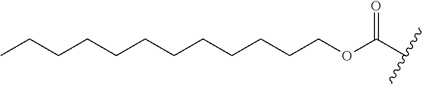 [SMILES: *C(=O)OCCCCCCCCCCCC] |
| phenyl | 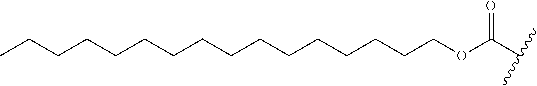 [SMILES: *C(=O)OCCCCCCCCCCCCCCCC] |
| phenyl | 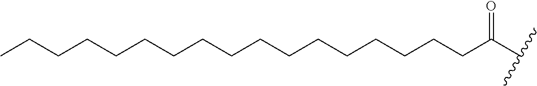 [SMILES: *C(=O)CCCCCCCCCCCCCCCCC] |
| phenyl | 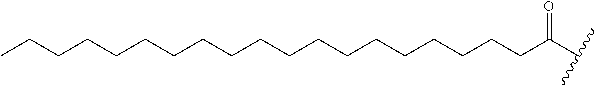 [SMILES: *C(=O)CCCCCCCCCCCCCCCCCCC] |
| phenyl | 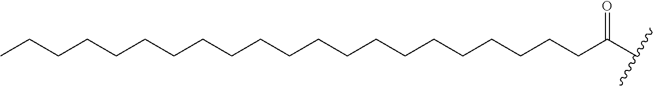 [SMILES: *C(=O)CCCCCCCCCCCCCCCCCCCCC] |
| phenyl | 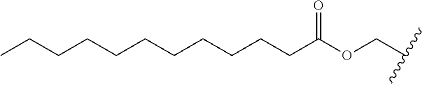 [SMILES: *COC(=O)CCCCCCCCCCC] |
| phenyl | 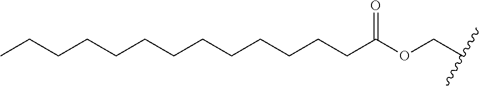 [SMILES: *COC(=O)CCCCCCCCCCCCC] |
| phenyl | 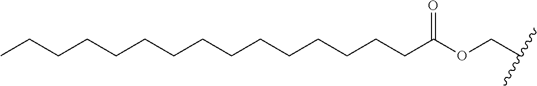 [SMILES: *COC(=O)CCCCCCCCCCCCCCC] |
| phenyl | 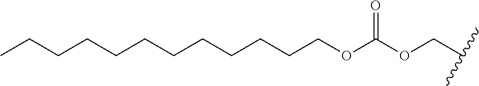 [SMILES: *COC(=O)OCCCCCCCCCCCC] |
| phenyl | 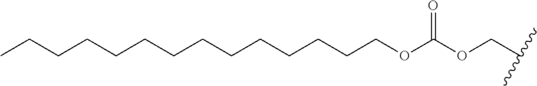 [SMILES: *COC(=O)OCCCCCCCCCCCCCC] |
| phenyl | 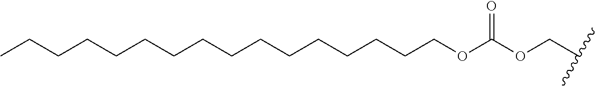 [SMILES: *COC(=O)OCCCCCCCCCCCCCCCC] |
| 4-chlorophenyl | 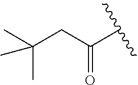 [SMILES: *C(=O)CC(C)(C)C] |
| 4-chlorophenyl | 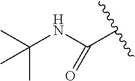 [SMILES: *C(=O)NC(C)(C)C] |
| 4-chlorophenyl | 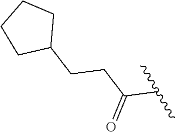 [SMILES: *C(=O)CCC1CCCC1] |
| 4-chlorophenyl | 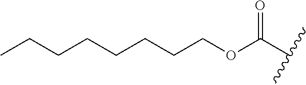 [SMILES: *C(=O)OCCCCCCCC] |
| 4-chlorophenyl | 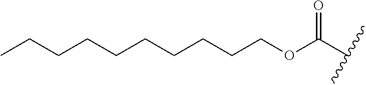 [SMILES: *C(=O)OCCCCCCCCCC] |
| 4-chlorophenyl | 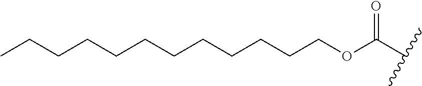 [SMILES: *C(=O)OCCCCCCCCCCCC] |
| 4-chlorophenyl | 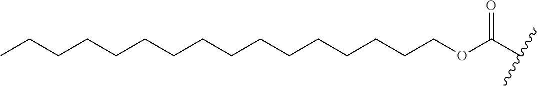 [SMILES: *C(=O)OCCCCCCCCCCCCCCCC] |
| 4-chlorophenyl | 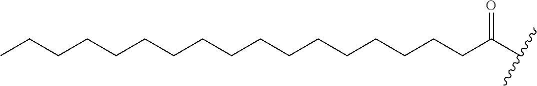 [SMILES: *C(=O)CCCCCCCCCCCCCCCCC] |
| 4-chlorophenyl | 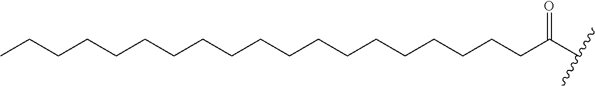 [SMILES: *C(=O)CCCCCCCCCCCCCCCCCCC] |
| 4-chlorophenyl | 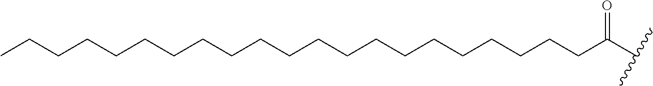 [SMILES: *C(=O)CCCCCCCCCCCCCCCCCCCCC] |
| 4-chlorophenyl | 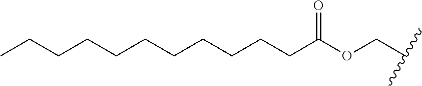 [SMILES: *COC(=O)CCCCCCCCCCC] |
| 4-chlorophenyl | 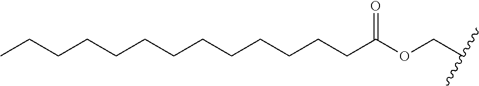 [SMILES: *COC(=O)CCCCCCCCCCCCC] |
| 4-chlorophenyl | 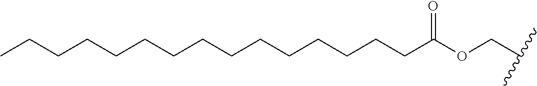 [SMILES: *COC(=O)CCCCCCCCCCCCCCC] |
| 4-chlorophenyl | 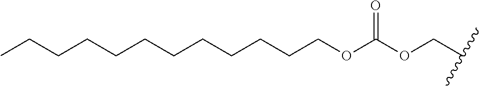 [SMILES: *COC(=O)OCCCCCCCCCCCC] |
| 4-chlorophenyl | 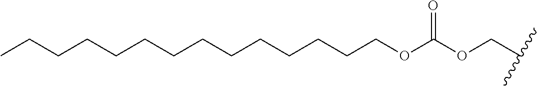 [SMILES: *COC(=O)OCCCCCCCCCCCCCC] |
| 4-chlorophenyl | 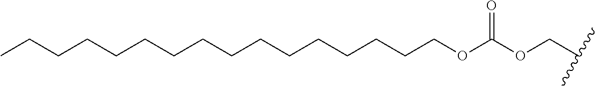 [SMILES: *COC(=O)OCCCCCCCCCCCCCCCC] |
| 3,4-dichlorophenyl | 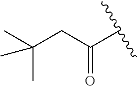 [SMILES: *C(=O)CC(C)(C)C] |
| 3,4-dichlorophenyl | 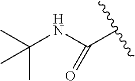 [SMILES: *C(=O)NC(C)(C)C] |
| 3,4-dichlorophenyl | 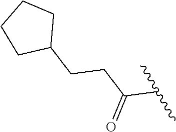 [SMILES: *C(=O)CCC1CCCC1] |
| 3,4-dichlorophenyl | 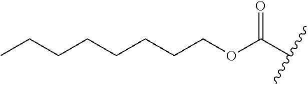 [SMILES: *C(=O)OCCCCCCCC] |
| 3,4-dichlorophenyl | 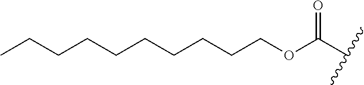 [SMILES: *C(=O)OCCCCCCCCCC] |
| 3,4-dichlorophenyl | 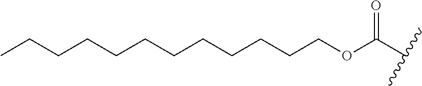 [SMILES: *C(=O)OCCCCCCCCCCCC] |
| 3,4-dichlorophenyl | 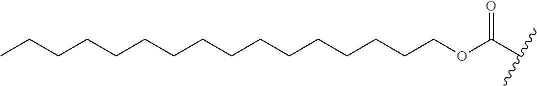 [SMILES: *C(=O)OCCCCCCCCCCCCCCCC] |
| 3,4-dichlorophenyl | 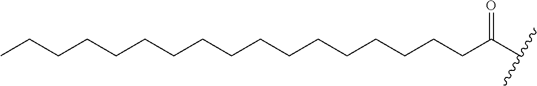 [SMILES: *C(=O)CCCCCCCCCCCCCCCCC] |
| 3,4-dichlorophenyl | 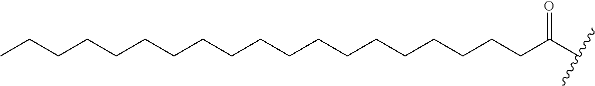 [SMILES: *C(=O)CCCCCCCCCCCCCCCCCCC] |
| 3,4-dichlorophenyl | 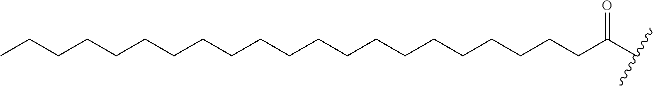 [SMILES: *C(=O)CCCCCCCCCCCCCCCCCCCCC] |
| 3,4-dichlorophenyl | 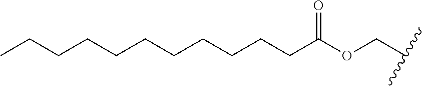 [SMILES: *COC(=O)CCCCCCCCCCC] |
| 3,4-dichlorophenyl | 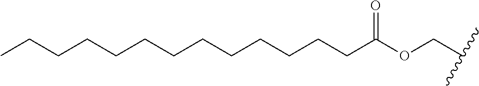 [SMILES: *COC(=O)CCCCCCCCCCCCC] |
| 3,4-dichlorophenyl | 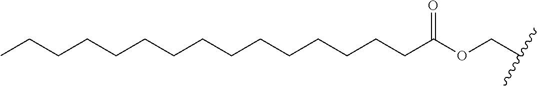 [SMILES: *COC(=O)CCCCCCCCCCCCCCC] |
| 3,4-dichlorophenyl | 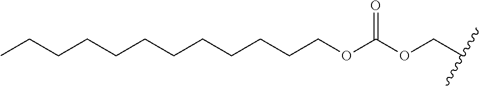 [SMILES: *COC(=O)OCCCCCCCCCCCC] |
| 3,4-dichlorophenyl | 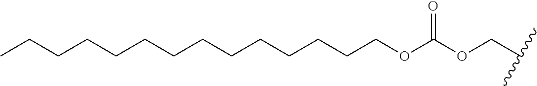 [SMILES: *COC(=O)OCCCCCCCCCCCCCC] |
| 3,4-dichlorophenyl | 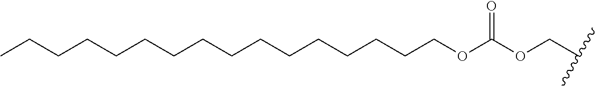 [SMILES: *COC(=O)OCCCCCCCCCCCCCCCC] |
| 4-methylphenyl | 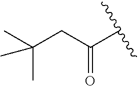 [SMILES: *C(=O)CC(C)(C)C] |
| 4-methylphenyl | 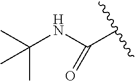 [SMILES: *C(=O)NC(C)(C)C] |
| 4-methylphenyl | 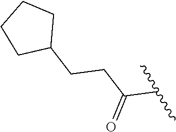 [SMILES: *C(=O)CCC1CCCC1] |
| 4-methylphenyl | 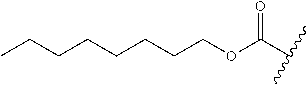 [SMILES: *C(=O)OCCCCCCCC] |
| 4-methylphenyl | 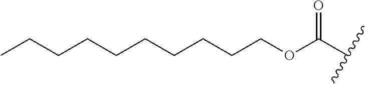 [SMILES: *C(=O)OCCCCCCCCCC] |
| 4-methylphenyl | 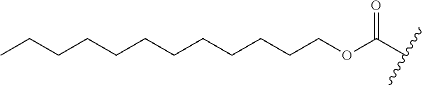 [SMILES: *C(=O)OCCCCCCCCCCCC] |
| 4-methylphenyl | 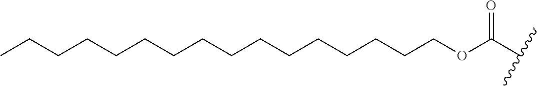 [SMILES: *C(=O)OCCCCCCCCCCCCCCCC] |
| 4-methylphenyl | 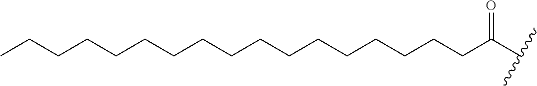 [SMILES: *C(=O)CCCCCCCCCCCCCCCCC] |
| 4-methylphenyl | 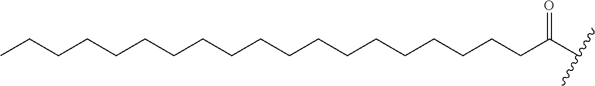 [SMILES: *C(=O)CCCCCCCCCCCCCCCCCCC] |
| 4-methylphenyl | 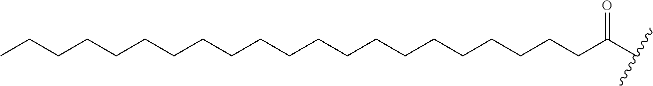 [SMILES: *C(=O)CCCCCCCCCCCCCCCCCCCCC] |
| 4-methylphenyl | 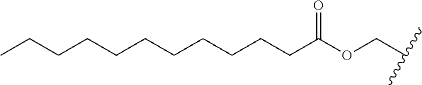 [SMILES: *COC(=O)CCCCCCCCCCC] |
| 4-methylphenyl | 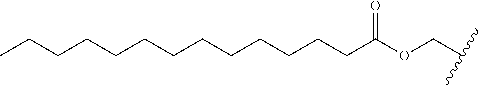 [SMILES: *COC(=O)CCCCCCCCCCCCC] |
| 4-methylphenyl | 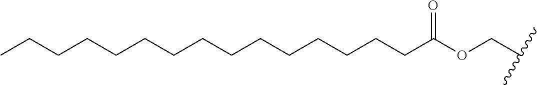 [SMILES: *COC(=O)CCCCCCCCCCCCCCC] |
| 4-methylphenyl | 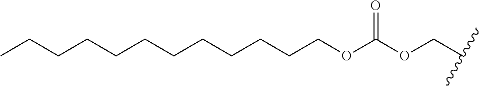 [SMILES: *COC(=O)OCCCCCCCCCCCC] |
| 4-methylphenyl | 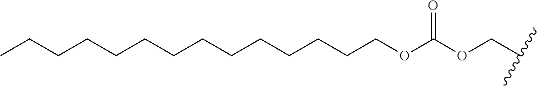 [SMILES: *COC(=O)OCCCCCCCCCCCCCC] |
| 4-methylphenyl | 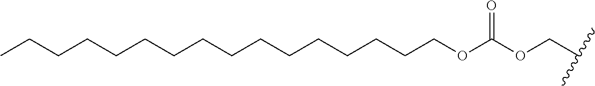 [SMILES: *COC(=O)OCCCCCCCCCCCCCCCC] |
| 4-methoxyphenyl | 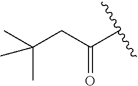 [SMILES: *C(=O)CC(C)(C)C] |
| 4-methoxyphenyl | 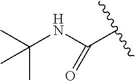 [SMILES: *C(=O)NC(C)(C)C] |
| 4-methoxyphenyl | 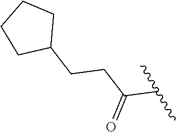 [SMILES: *C(=O)CCC1CCCC1] |
| 4-methoxyphenyl | 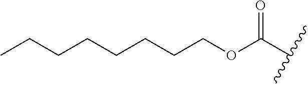 [SMILES: *C(=O)OCCCCCCCC] |
| 4-methoxyphenyl | 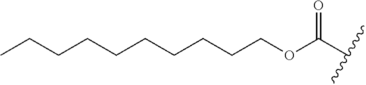 [SMILES: *C(=O)OCCCCCCCCCC] |
| 4-methoxyphenyl | 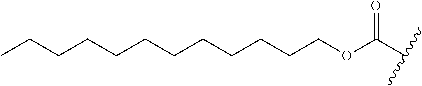 [SMILES: *C(=O)OCCCCCCCCCCCC] |
| 4-methoxyphenyl | 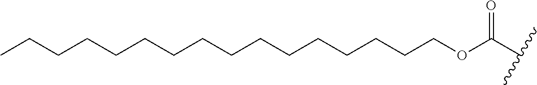 [SMILES: *C(=O)OCCCCCCCCCCCCCCCC] |
| 4-methoxyphenyl | 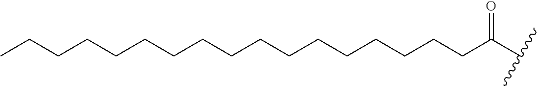 [SMILES: *C(=O)CCCCCCCCCCCCCCCCC] |
| 4-methoxyphenyl | 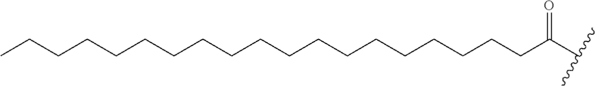 [SMILES: *C(=O)CCCCCCCCCCCCCCCCCCC] |
| 4-methoxyphenyl | 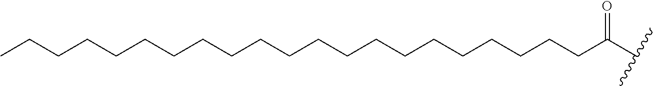 [SMILES: *C(=O)CCCCCCCCCCCCCCCCCCCCC] |
| 4-methoxyphenyl | 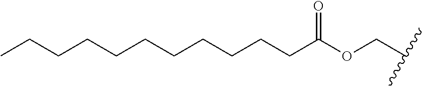 [SMILES: *COC(=O)CCCCCCCCCCC] |
| 4-methoxyphenyl | 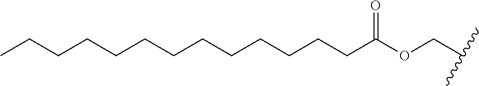 [SMILES: *COC(=O)CCCCCCCCCCCCC] |
| 4-methoxyphenyl | 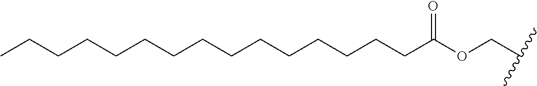 [SMILES: *COC(=O)CCCCCCCCCCCCCCC] |
| 4-methoxyphenyl | 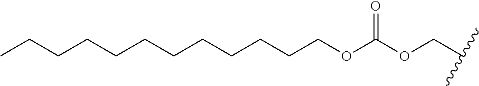 [SMILES: *COC(=O)OCCCCCCCCCCCC] |
| 4-methoxyphenyl | 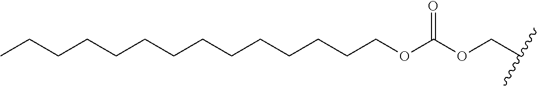 [SMILES: *COC(=O)OCCCCCCCCCCCCCC] |
| 4-methoxyphenyl | 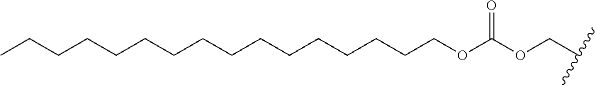 [SMILES: *COC(=O)OCCCCCCCCCCCCCCCC] |

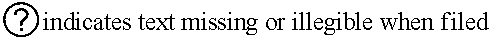

In a 49^{th} aspect, provided herein is a compound, or pharmaceutically acceptable salt thereof, having a structure provided in Formula (I-16),

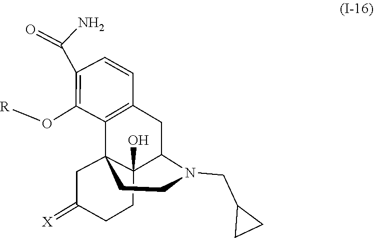

wherein X is ═O, R is R⁴—O—C(O)—O—CH(R³)— and R³ and R⁴ are any of the pair of R³/R⁴ groups recited in Table 6. Alternatively, X is ═CH₂, R is R⁴—O—C(O)—O—CH(R³)— and R³ and R⁴ are any of the pair of R³/R⁴ groups recited in Table 6.

In a 50^{th} aspect, provided herein is a compound, or pharmaceutically acceptable salt thereof, having a structure provided in Formula (II-16),

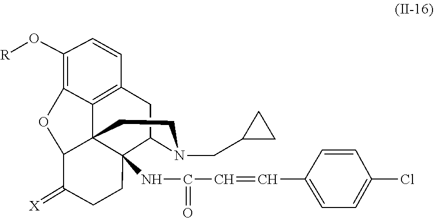

wherein X is ═O, R is R⁴—O—C(O)—O—CH(R³)— and R³ and R⁴ are any of the pair of R³/R⁴ groups recited in Table 6. Alternatively, X is ═CH₂, R is R⁴—O—C(O)—O—CH(R³)— and R³ and R⁴ are any of the pair of R³/R⁴ groups recited in Table 6.

In a 51^{st} aspect, provided herein is a compound, or pharmaceutically acceptable salt thereof, having a structure provided in Formula (III-16),

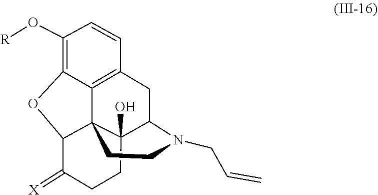

wherein X is ═O, R is R⁴—O—C(O)—O—CH(R³)— and R³ and R⁴ are any of the pair of R³/R⁴ groups recited in Table 6. Alternatively, X is ═CH₂, R is R⁴—O—C(O)—O—CH(R³)— and R³ and R⁴ are any of the pair of R³/R⁴ groups recited in Table 6.

TABLE 6

| R³ | R⁴ |
|---|---|
| H | Me |
| H | —C₇H₁₅ |
| H | —C₉H₁₉ |
| H | —C₁₁H₂₃ |
| H | —C₁₃H₂₇ |
| H | —C₁₅H₃₁ |
| H | —C₁₇H₃₅ |
| H | 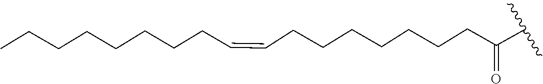 [SMILES: *C(=O)CCCCCCC/C=C\CCCCCCCC] |
| H | 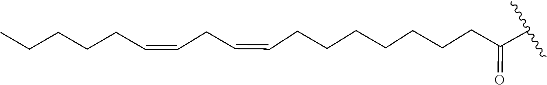 [SMILES: *C(=O)CCCCCCC/C=C\C/C=C\CCCCC] |
| H | 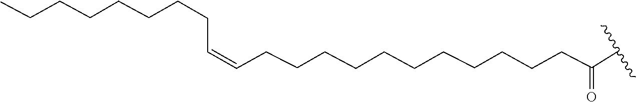 [SMILES: *C(=O)CCCCCCCCCCC/C=C\CCCCCCCC] |
| H | 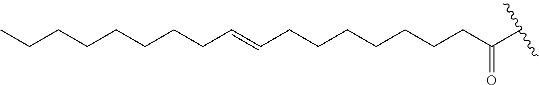 [SMILES: *C(=O)CCCCCCC/C=C/CCCCCCCC] |
| H | 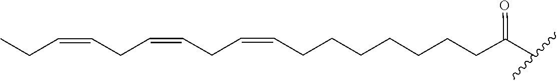 [SMILES: *C(=O)CCCCCCC/C=C\C/C=C\C/C=C\CC] |
| H | tBu |
| H | phenyl |
| H | 4-chlorophenyl |
| H | 3,4-dichlorophenyl |
| H | 4-methylphenyl |
| H | 4-methoxyphenyl |
| Me | Me |
| Me | —C₇H₁₅ |
| Me | —C₉H₁₉ |
| Me | —C₁₁H₂₃ |
| Me | —C₁₃H₂₇ |
| Me | —C₁₅H₃₁ |
| Me | —C₁₇H₃₅ |
| Me | 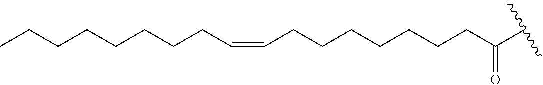 [SMILES: *C(=O)CCCCCCC/C=C\CCCCCCCC] |
| Me | 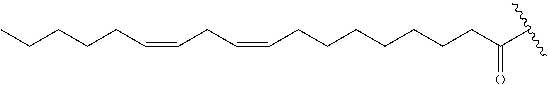 [SMILES: *C(=O)CCCCCCC/C=C\C/C=C\CCCCC] |
| Me | 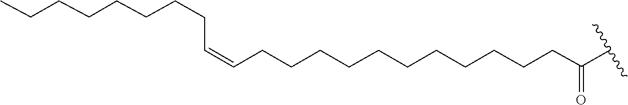 [SMILES: *C(=O)CCCCCCCCCCC/C=C\CCCCCCCC] |
| Me | 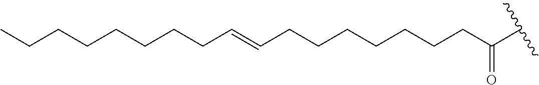 [SMILES: *C(=O)CCCCCCC/C=C/CCCCCCCC] |
| Me | 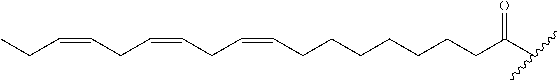 [SMILES: *C(=O)CCCCCCC/C=C\C/C=C\C/C=C\CC] |
| Me | tBu |
| Me | phenyl |
| Me | 4-chlorophenyl |
| Me | 3,4-dichlorophenyl |
| Me | 4-methylphenyl |
| Me | 4-methoxyphenyl |
| tBu | Me |
| tBu | —C₇H₁₅ |
| tBu | —C₉H₁₉ |
| tBu | —C₁₁H₂₃ |
| tBu | —C₁₃H₂₇ |
| tBu | —C₁₅H₃₁ |
| tBu | —C₁₇H₃₅ |
| tBu | 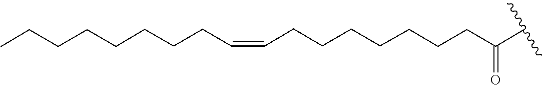 [SMILES: *C(=O)CCCCCCC/C=C\CCCCCCCC] |
| tBu | 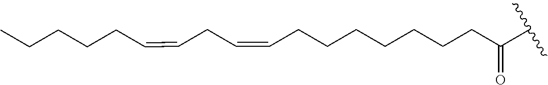 [SMILES: *C(=O)CCCCCCC/C=C\C/C=C\CCCCC] |
| tBu | 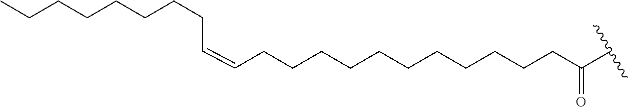 [SMILES: *C(=O)CCCCCCCCCCC/C=C\CCCCCCCC] |
| tBu | 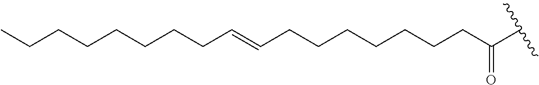 [SMILES: *C(=O)CCCCCCC/C=C/CCCCCCCC] |
| tBu | 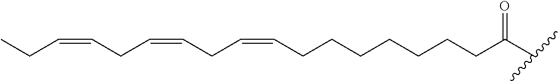 [SMILES: *C(=O)CCCCCCC/C=C\C/C=C\C/C=C\CC] |
| tBu | phenyl |
| tBu | tBu |
| tBu | 4-chlorophenyl |
| tBu | 3,4-dichlorophenyl |
| tBu | 4-methylphenyl |
| tBu | 4-methoxyphenyl |
| —C₇H₁₅ | Me |
| —C₇H₁₅ | —C₇H₁₅ |
| —C₇H₁₅ | —C₉H₁₉ |
| —C₇H₁₅ | —C₁₁H₂₃ |
| —C₇H₁₅ | —C₁₃H₂₇ |
| —C₇H₁₅ | —C₁₅H₃₁ |
| —C₇H₁₅ | —C₁₇H₃₅ |
| —C₇H₁₅ | 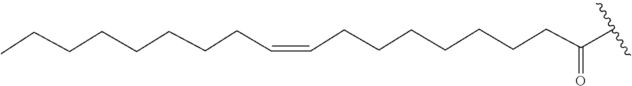 [SMILES: *C(=O)CCCCCCC/C=C\CCCCCCCC] |
| —C₇H₁₅ | 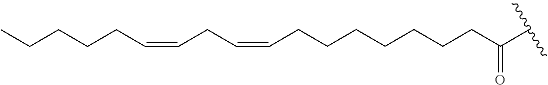 [SMILES: *C(=O)CCCCCCC/C=C\C/C=C\CCCCC] |
| —C₇H₁₅ | 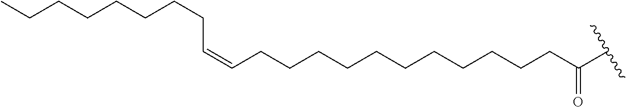 [SMILES: *C(=O)CCCCCCCCCCC/C=C\CCCCCCCC] |
| —C₇H₁₅ | 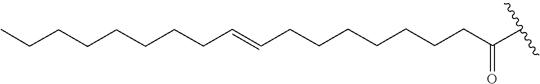 [SMILES: *C(=O)CCCCCCC/C=C/CCCCCCCC] |
| —C₇H₁₅ | 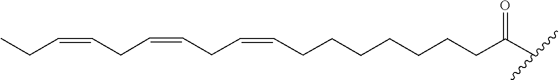 [SMILES: *C(=O)CCCCCCC/C=C\C/C=C\C/C=C\CC] |
| —C₇H₁₅ | tBu |
| —C₇H₁₅ | phenyl |
| —C₇H₁₅ | 4-chlorophenyl |
| —C₇H₁₅ | 3,4-dichlorophenyl |
| —C₇H₁₅ | 4-methylphenyl |
| —C₇H₁₅ | 4-methoxyphenyl |
| —C₉H₁₉ | Me |
| —C₉H₁₉ | —C₇H₁₅ |
| —C₉H₁₉ | —C₉H₁₉ |
| —C₉H₁₉ | —C₁₁H₂₃ |
| —C₉H₁₉ | —C₁₃H₂₇ |
| —C₉H₁₉ | —C₁₅H₃₁ |
| —C₉H₁₉ | —C₁₇H₃₅ |
| —C₉H₁₉ | 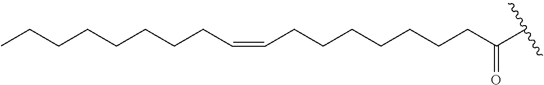 [SMILES: *C(=O)CCCCCCC/C=C\CCCCCCCC] |
| —C₉H₁₉ | 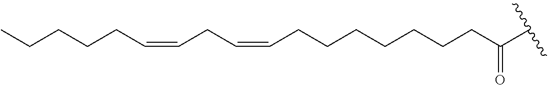 [SMILES: *C(=O)CCCCCCC/C=C\C/C=C\CCCCC] |
| —C₉H₁₉ | 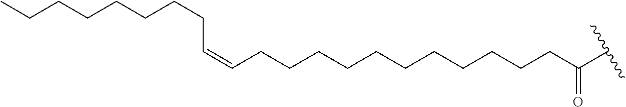 [SMILES: *C(=O)CCCCCCCCCCC/C=C\CCCCCCCC] |
| —C₉H₁₉ | 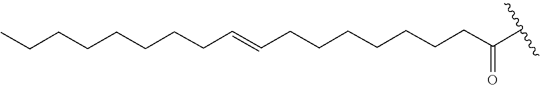 [SMILES: *C(=O)CCCCCCC/C=C/CCCCCCCC] |
| —C₉H₁₉ | 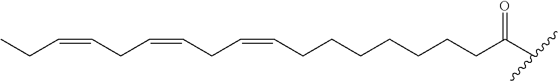 [SMILES: *C(=O)CCCCCCC/C=C\C/C=C\C/C=C\CC] |
| —C₉H₁₉ | tBu |
| —C₉H₁₉ | phenyl |
| —C₉H₁₉ | 4-chlorophenyl |
| —C₉H₁₉ | 3,4-dichlorophenyl |
| —C₉H₁₉ | 4-methylphenyl |
| —C₉H₁₉ | 4-methoxyphenyl |
| —C₁₁H₂₃ | Me |
| —C₁₁H₂₃ | —C₇H₁₅ |
| —C₁₁H₂₃ | —C₉H₁₉ |
| —C₁₁H₂₃ | —C₁₁H₂₃ |
| —C₁₁H₂₃ | —C₁₃H₂₇ |
| —C₁₁H₂₃ | —C₁₅H₃₁ |
| —C₁₁H₂₃ | —C₁₇H₃₅ |
| —C₁₁H₂₃ | 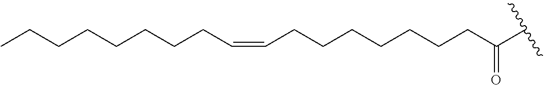 [SMILES: *C(=O)CCCCCCC/C=C\CCCCCCCC] |
| —C₁₁H₂₃ | 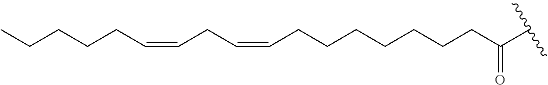 [SMILES: *C(=O)CCCCCCC/C=C\C/C=C\CCCCC] |
| —C₁₁H₂₃ | 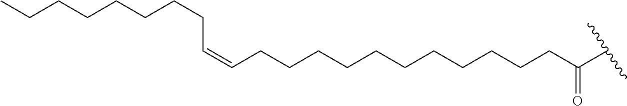 [SMILES: *C(=O)CCCCCCCCCCC/C=C\CCCCCCCC] |
| —C₁₁H₂₃ | 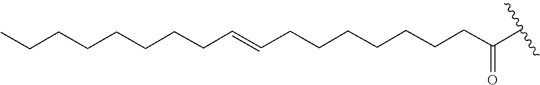 [SMILES: *C(=O)CCCCCCC/C=C/CCCCCCCC] |
| —C₁₁H₂₃ | 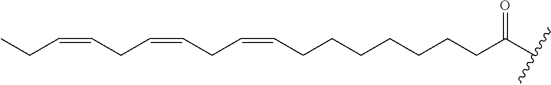 [SMILES: *C(=O)CCCCCCC/C=C\C/C=C\C/C=C\CC] |
| —C₁₁H₂₃ | tBu |
| —C₁₁H₂₃ | phenyl |
| —C₁₁H₂₃ | 4-chlorophenyl |
| —C₁₁H₂₃ | 3,4-dichlorophenyl |
| —C₁₁H₂₃ | 4-methylphenyl |
| —C₁₁H₂₃ | 4-methoxyphenyl |
| —C₁₃H₂₇ | Me |
| —C₁₃H₂₇ | —C₇H₁₅ |
| —C₁₃H₂₇ | —C₉H₁₉ |
| —C₁₃H₂₇ | —C₁₁H₂₃ |
| —C₁₃H₂₇ | —C₁₃H₂₇ |
| —C₁₃H₂₇ | —C₁₅H₃₁ |
| —C₁₃H₂₇ | —C₁₇H₃₅ |
| —C₁₃H₂₇ | 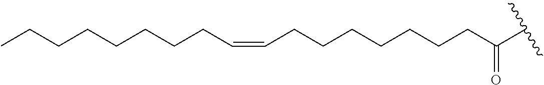 [SMILES: *C(=O)CCCCCCC/C=C\CCCCCCCC] |
| —C₁₃H₂₇ | 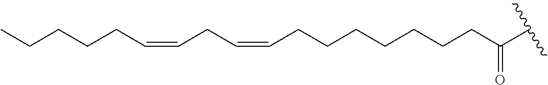 [SMILES: *C(=O)CCCCCCC/C=C\C/C=C\CCCCC] |
| —C₁₃H₂₇ | 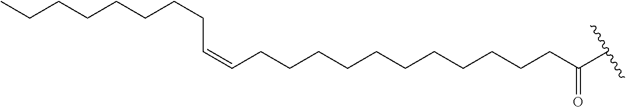 [SMILES: *C(=O)CCCCCCCCCCC/C=C\CCCCCCCC] |
| —C₁₃H₂₇ | 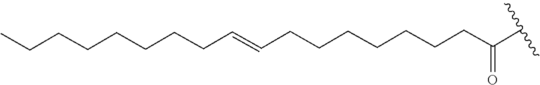 [SMILES: *C(=O)CCCCCCC/C=C/CCCCCCCC] |
| —C₁₃H₂₇ | 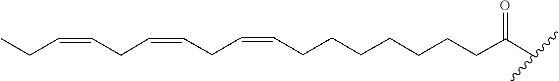 [SMILES: *C(=O)CCCCCCC/C=C\C/C=C\C/C=C\CC] |
| —C₁₃H₂₇ | tBu |
| —C₁₃H₂₇ | phenyl |
| —C₁₃H₂₇ | 4-chlorophenyl |
| —C₁₃H₂₇ | 3,4-dichlorophenyl |
| —C₁₃H₂₇ | 4-methylphenyl |
| —C₁₃H₂₇ | 4-methoxyphenyl |
| —C₁₅H₃₁ | Me |
| —C₁₅H₃₁ | —C₇H₁₅ |
| —C₁₅H₃₁ | —C₉H₁₉ |
| —C₁₅H₃₁ | —C₁₁H₂₃ |
| —C₁₅H₃₁ | —C₁₃H₂₇ |
| —C₁₅H₃₁ | —C₁₅H₃₁ |
| —C₁₅H₃₁ | —C₁₇H₃₅ |
| —C₁₅H₃₁ | 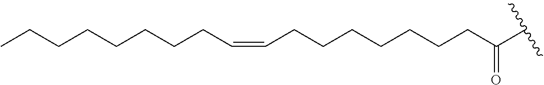 [SMILES: *C(=O)CCCCCCC/C=C\CCCCCCCC] |
| —C₁₅H₃₁ | 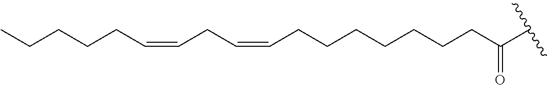 [SMILES: *C(=O)CCCCCCC/C=C\C/C=C\CCCCC] |
| —C₁₅H₃₁ | 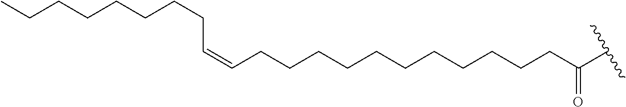 [SMILES: *C(=O)CCCCCCCCCCC/C=C\CCCCCCCC] |
| —C₁₅H₃₁ | 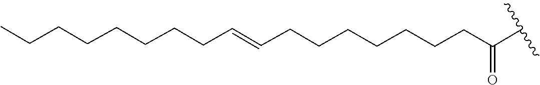 [SMILES: *C(=O)CCCCCCC/C=C/CCCCCCCC] |
| —C₁₅H₃₁ | 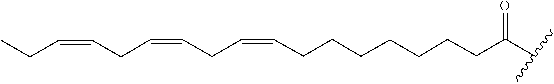 [SMILES: *C(=O)CCCCCCC/C=C\C/C=C\C/C=C\CC] |
| —C₁₅H₃₁ | tBu |
| —C₁₅H₃₁ | phenyl |
| —C₁₅H₃₁ | 4-chlorophenyl |
| —C₁₅H₃₁ | 3,4-dichlorophenyl |
| —C₁₅H₃₁ | 4-methylphenyl |
| —C₁₅H₃₁ | 4-methoxyphenyl |
| —C₁₇H₃₅ | Me |
| —C₁₇H₃₅ | —C₇H₁₅ |
| —C₁₇H₃₅ | —C₉H₁₉ |
| —C₁₇H₃₅ | —C₁₁H₂₃ |
| —C₁₇H₃₅ | —C₁₃H₂₇ |
| —C₁₇H₃₅ | —C₁₅H₃₁ |
| —C₁₇H₃₅ | —C₁₇H₃₅ |
| —C₁₇H₃₅ | 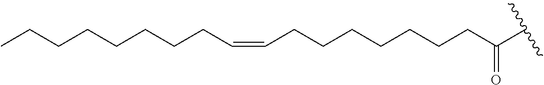 [SMILES: *C(=O)CCCCCCC/C=C\CCCCCCCC] |
| —C₁₇H₃₅ | 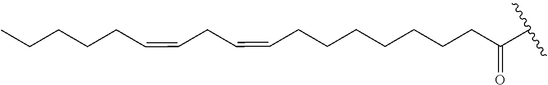 [SMILES: *C(=O)CCCCCCC/C=C\C/C=C\CCCCC] |
| —C₁₇H₃₅ | 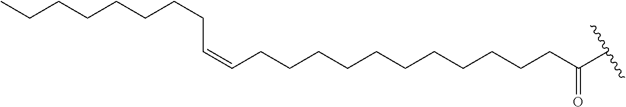 [SMILES: *C(=O)CCCCCCCCCCC/C=C\CCCCCCCC] |
| —C₁₇H₃₅ | 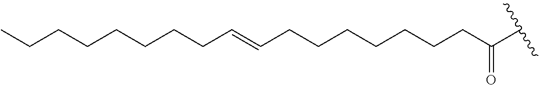 [SMILES: *C(=O)CCCCCCC/C=C/CCCCCCCC] |
| —C₁₇H₃₅ | 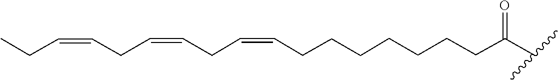 [SMILES: *C(=O)CCCCCCC/C=C\C/C=C\C/C=C\CC] |
| —C₁₇H₃₅ | tBu |
| —C₁₇H₃₅ | phenyl |
| —C₁₇H₃₅ | 4-chlorophenyl |
| —C₁₇H₃₅ | 3,4-dichlorophenyl |
| —C₁₇H₃₅ | 4-methylphenyl |
| —C₁₇H₃₅ | 4-methoxyphenyl |
| Phenyl | Me |
| Phenyl | —C₇H₁₅ |
| Phenyl | —C₉H₁₉ |
| Phenyl | —C₁₁H₂₃ |
| Phenyl | —C₁₃H₂₇ |
| Phenyl | —C₁₅H₃₁ |
| Phenyl | —C₁₇H₃₅ |
| Phenyl | 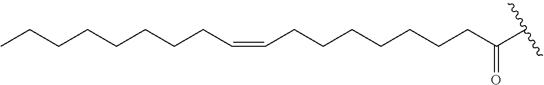 [SMILES: *C(=O)CCCCCCC/C=C\CCCCCCCC] |
| Phenyl | 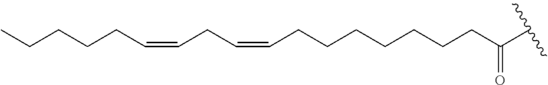 [SMILES: *C(=O)CCCCCCC/C=C\C/C=C\CCCCC] |
| Phenyl | 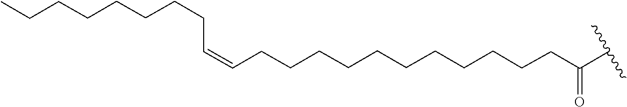 [SMILES: *C(=O)CCCCCCCCCCC/C=C\CCCCCCCC] |
| Phenyl | 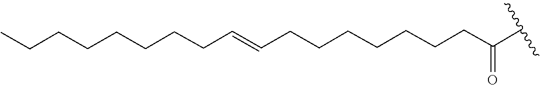 [SMILES: *C(=O)CCCCCCC/C=C/CCCCCCCC] |
| Phenyl | 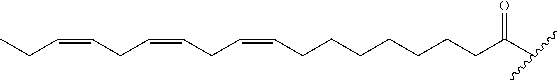 [SMILES: *C(=O)CCCCCCC/C=C\C/C=C\C/C=C\CC] |
| Phenyl | tBu |
| Phenyl | phenyl |
| Phenyl | 4-chlorophenyl |
| Phenyl | 3,4-dichlorophenyl |
| Phenyl | 4-methylphenyl |
| Phenyl | 4-methoxyphenyl |
| 4-chlorophenyl | Me |
| 4-chlorophenyl | —C₇H₁₅ |
| 4-chlorophenyl | —C₉H₁₉ |
| 4-chlorophenyl | —C₁₁H₂₃ |
| 4-chlorophenyl | —C₁₃H₂₇ |
| 4-chlorophenyl | —C₁₅H₃₁ |
| 4-chlorophenyl | —C₁₇H₃₅ |
| 4-chlorophenyl | 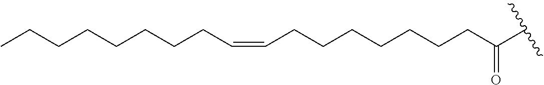 [SMILES: *C(=O)CCCCCCC/C=C\CCCCCCCC] |
| 4-chlorophenyl | 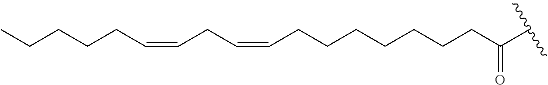 [SMILES: *C(=O)CCCCCCC/C=C\C/C=C\CCCCC] |
| 4-chlorophenyl | 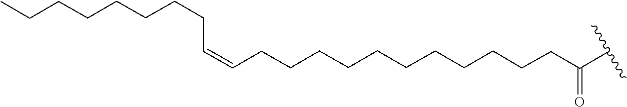 [SMILES: *C(=O)CCCCCCCCCCC/C=C\CCCCCCCC] |
| 4-chlorophenyl | 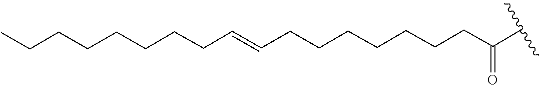 [SMILES: *C(=O)CCCCCCC/C=C/CCCCCCCC] |
| 4-chlorophenyl | 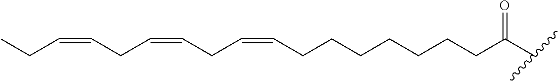 [SMILES: *C(=O)CCCCCCC/C=C\C/C=C\C/C=C\CC] |
| 4-chlorophenyl | tBu |
| 4-chlorophenyl | phenyl |
| 4-chlorophenyl | 4-chlorophenyl |
| 4-chlorophenyl | 3,4-dichlorophenyl |
| 4-chlorophenyl | 4-methylphenyl |
| 4-chlorophenyl | 4-methoxyphenyl |
| 3,4-dichlorophenyl | Me |
| 3,4-dichlorophenyl | —C₇H₁₅ |
| 3,4-dichlorophenyl | —C₉H₁₉ |
| 3,4-dichlorophenyl | —C₁₁H₂₃ |
| 3,4-dichlorophenyl | —C₁₃H₂₇ |
| 3,4-dichlorophenyl | —C₁₅H₃₁ |
| 3,4-dichlorophenyl | —C₁₇H₃₅ |
| 3,4-dichlorophenyl | 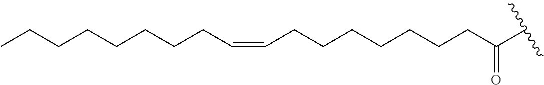 [SMILES: *C(=O)CCCCCCC/C=C\CCCCCCCC] |
| 3,4-dichlorophenyl | 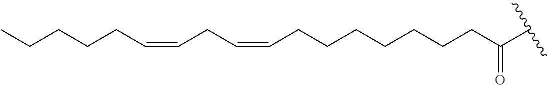 [SMILES: *C(=O)CCCCCCC/C=C\C/C=C\CCCCC] |
| 3,4-dichlorophenyl | 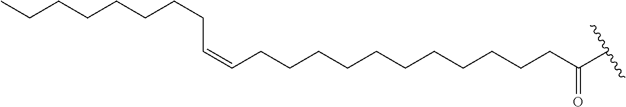 [SMILES: *C(=O)CCCCCCCCCCC/C=C\CCCCCCCC] |
| 3,4-dichlorophenyl | 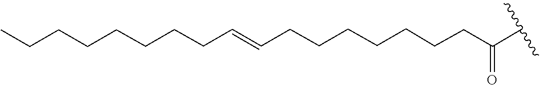 [SMILES: *C(=O)CCCCCCC/C=C/CCCCCCCC] |
| 3,4-dichlorophenyl | 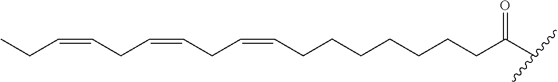 [SMILES: *C(=O)CCCCCCC/C=C\C/C=C\C/C=C\CC] |
| 3,4-dichlorophenyl | tBu |
| 3,4-dichlorophenyl | phenyl |
| 3,4-dichlorophenyl | 4-chlorophenyl |
| 3,4-dichlorophenyl | 3,4-dichlorophenyl |
| 3,4-dichlorophenyl | 4-methylphenyl |
| 3,4-dichlorophenyl | 4-methoxyphenyl |
| 4-methylphenyl | Me |
| 4-methylphenyl | —C₇H₁₅ |
| 4-methylphenyl | —C₉H₁₉ |
| 4-methylphenyl | —C₁₁H₂₃ |
| 4-methylphenyl | —C₁₃H₂₇ |
| 4-methylphenyl | —C₁₅H₃₁ |
| 4-methylphenyl | —C₁₇H₃₅ |
| 4-methylphenyl | 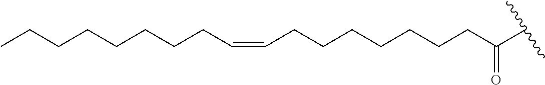 [SMILES: *C(=O)CCCCCCC/C=C\CCCCCCCC] |
| 4-methylphenyl | 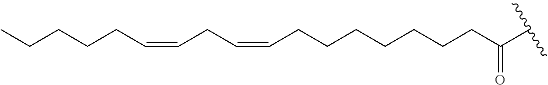 [SMILES: *C(=O)CCCCCCC/C=C\C/C=C\CCCCC] |
| 4-methylphenyl | 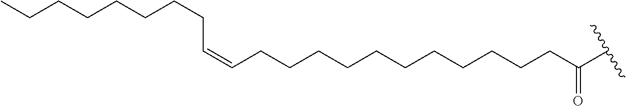 [SMILES: *C(=O)CCCCCCCCCCC/C=C\CCCCCCCC] |
| 4-methylphenyl | 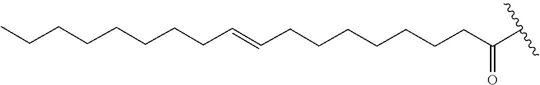 [SMILES: *C(=O)CCCCCCC/C=C/CCCCCCCC] |
| 4-methylphenyl | 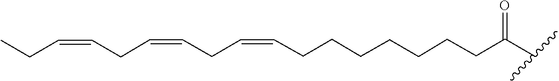 [SMILES: *C(=O)CCCCCCC/C=C\C/C=C\C/C=C\CC] |
| 4-methylphenyl | tBu |
| 4-methylphenyl | phenyl |
| 4-methylphenyl | 4-chlorophenyl |
| 4-methylphenyl | 3,4-dichlorophenyl |
| 4-methylphenyl | 4-methylphenyl |
| 4-methylphenyl | 4-methoxyphenyl |
| 4-methoxyphenyl | Me |
| 4-methoxyphenyl | —C₇H₁₅ |
| 4-methoxyphenyl | —C₉H₁₉ |
| 4-methoxyphenyl | —C₁₁H₂₃ |
| 4-methoxyphenyl | —C₁₃H₂₇ |
| 4-methoxyphenyl | —C₁₅H₃₁ |
| 4-methoxyphenyl | —C₁₇H₃₅ |
| 4-methoxyphenyl | 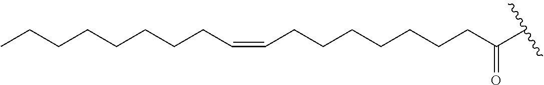 [SMILES: *C(=O)CCCCCCC/C=C\CCCCCCCC] |
| 4-methoxyphenyl | 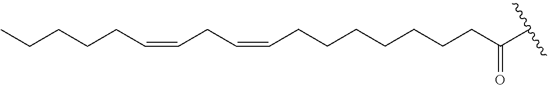 [SMILES: *C(=O)CCCCCCC/C=C\C/C=C\CCCCC] |
| 4-methoxyphenyl | 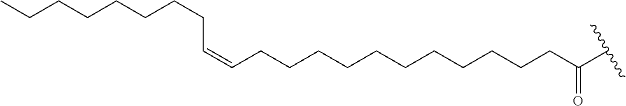 [SMILES: *C(=O)CCCCCCCCCCC/C=C\CCCCCCCC] |
| 4-methoxyphenyl | 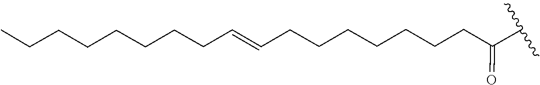 [SMILES: *C(=O)CCCCCCC/C=C/CCCCCCCC] |
| 4-methoxyphenyl | 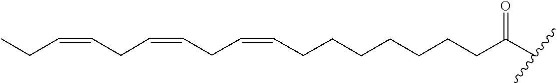 [SMILES: *C(=O)CCCCCCC/C=C\C/C=C\C/C=C\CC] |
| 4-methoxyphenyl | tBu |
| 4-methoxyphenyl | phenyl |
| 4-methoxyphenyl | 4-chlorophenyl |
| 4-methoxyphenyl | 3,4-dichlorophenyl |
| 4-methoxyphenyl | 4-methylphenyl |
| 4-methoxyphenyl | 4-methoxyphenyl |
| 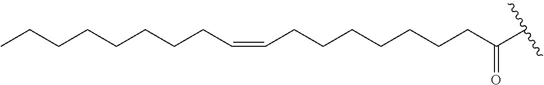 [SMILES: *C(=O)CCCCCCC/C=C\CCCCCCCC] | Me |
| 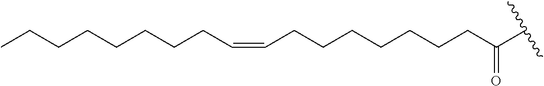 [SMILES: *C(=O)CCCCCCC/C=C\CCCCCCCC] | —C₇H₁₅ |
| 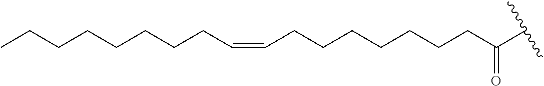 [SMILES: *C(=O)CCCCCCC/C=C\CCCCCCCC] | —C₉H₁₉ |
| 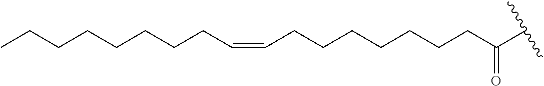 [SMILES: *C(=O)CCCCCCC/C=C\CCCCCCCC] | —C₁₁H₂₃ |
| 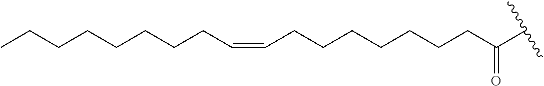 [SMILES: *C(=O)CCCCCCC/C=C\CCCCCCCC] | —C₁₃H₂₇ |
| 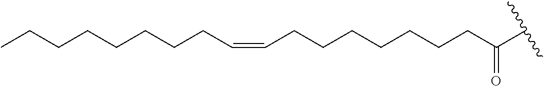 [SMILES: *C(=O)CCCCCCC/C=C\CCCCCCCC] | —C₁₅H₃₁ |
| 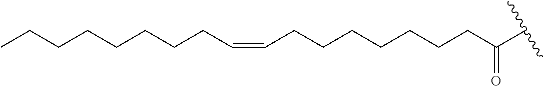 [SMILES: *C(=O)CCCCCCC/C=C\CCCCCCCC] | —C₁₇H₃₅ |
| 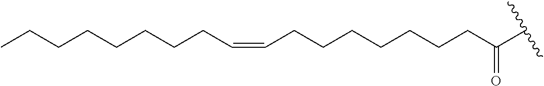 [SMILES: *C(=O)CCCCCCC/C=C\CCCCCCCC] | 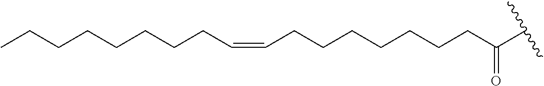 [SMILES: *C(=O)CCCCCCC/C=C\CCCCCCCC] |
| 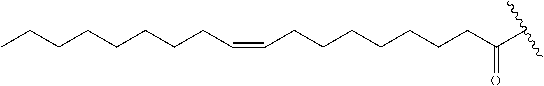 [SMILES: *C(=O)CCCCCCC/C=C\CCCCCCCC] | 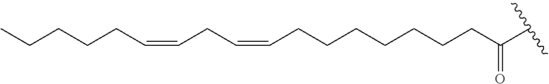 [SMILES: *C(=O)CCCCCCC/C=C\C/C=C\CCCCC] |
| 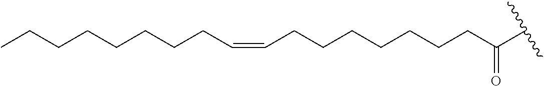 [SMILES: *C(=O)CCCCCCC/C=C\CCCCCCCC] | 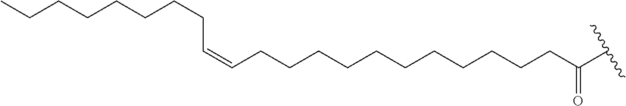 [SMILES: *C(=O)CCCCCCCCCCC/C=C\CCCCCCCC] |
| 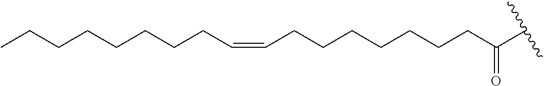 [SMILES: *C(=O)CCCCCCC/C=C\CCCCCCCC] | 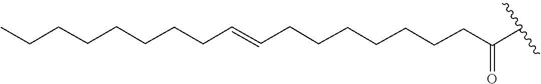 [SMILES: *C(=O)CCCCCCC/C=C/CCCCCCCC] |
| 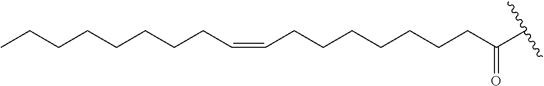 [SMILES: *C(=O)CCCCCCC/C=C\CCCCCCCC] | 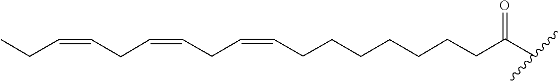 [SMILES: *C(=O)CCCCCCC/C=C\C/C=C\C/C=C\CC] |
| 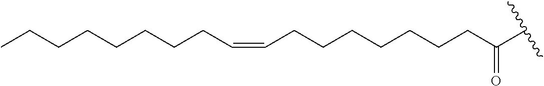 [SMILES: *C(=O)CCCCCCC/C=C\CCCCCCCC] | tBu |
| 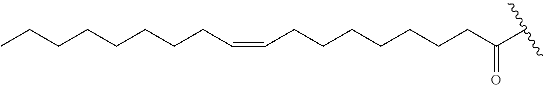 [SMILES: *C(=O)CCCCCCC/C=C\CCCCCCCC] | phenyl |
| 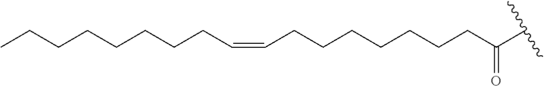 [SMILES: *C(=O)CCCCCCC/C=C\CCCCCCCC] | 4-chlorophenyl |
| 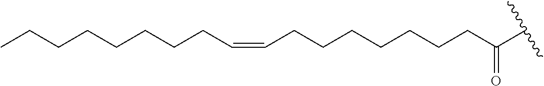 [SMILES: *C(=O)CCCCCCC/C=C\CCCCCCCC] | 3,4-dichlorophenyl |
| 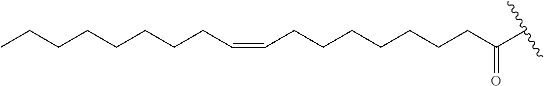 [SMILES: *C(=O)CCCCCCC/C=C\CCCCCCCC] | 4-methylphenyl |
| 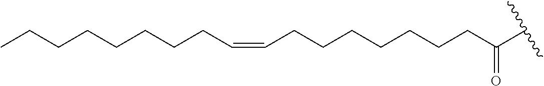 [SMILES: *C(=O)CCCCCCC/C=C\CCCCCCCC] | 4-methoxyphenyl |
| 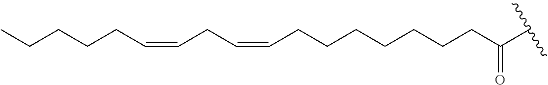 [SMILES: *C(=O)CCCCCCC/C=C\C/C=C\CCCCC] | Me |
| 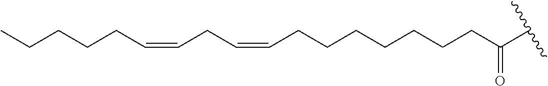 [SMILES: *C(=O)CCCCCCC/C=C\C/C=C\CCCCC] | —C₇H₁₅ |
| 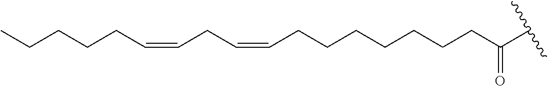 [SMILES: *C(=O)CCCCCCC/C=C\C/C=C\CCCCC] | —C₉H₁₉ |
| 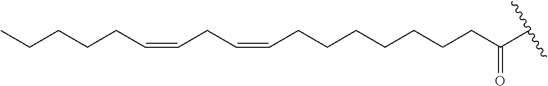 [SMILES: *C(=O)CCCCCCC/C=C\C/C=C\CCCCC] | —C₁₁H₂₃ |
| 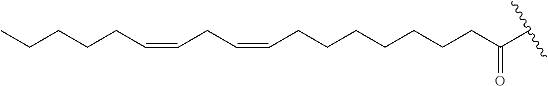 [SMILES: *C(=O)CCCCCCC/C=C\C/C=C\CCCCC] | —C₁₃H₂₇ |
| 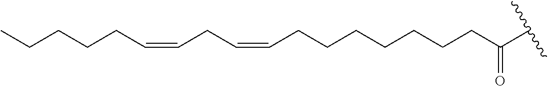 [SMILES: *C(=O)CCCCCCC/C=C\C/C=C\CCCCC] | —C₁₅H₃₁ |
| 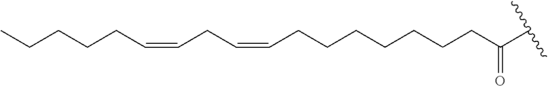 [SMILES: *C(=O)CCCCCCC/C=C\C/C=C\CCCCC] | —C₁₇H₃₅ |
| 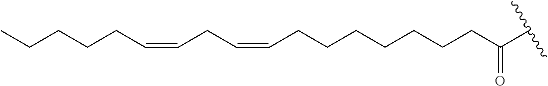 [SMILES: *C(=O)CCCCCCC/C=C\C/C=C\CCCCC] | 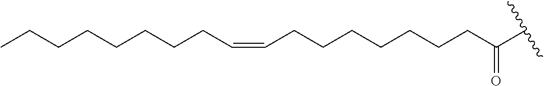 [SMILES: *C(=O)CCCCCCC/C=C\CCCCCCCC] |
| 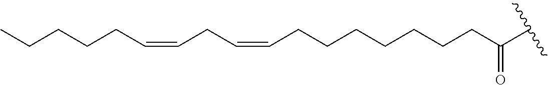 [SMILES: *C(=O)CCCCCCC/C=C\C/C=C\CCCCC] | 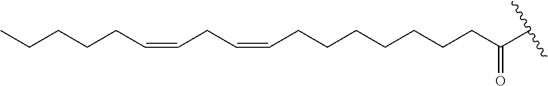 [SMILES: *C(=O)CCCCCCC/C=C\C/C=C\CCCCC] |
| 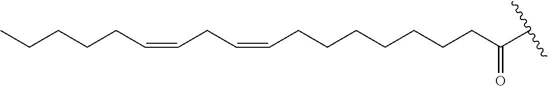 [SMILES: *C(=O)CCCCCCC/C=C\C/C=C\CCCCC] | 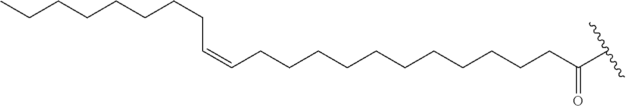 [SMILES: *C(=O)CCCCCCCCCCC/C=C\CCCCCCCC] |
| 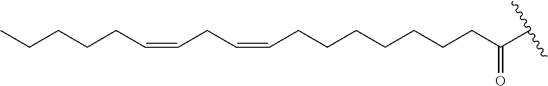 [SMILES: *C(=O)CCCCCCC/C=C\C/C=C\CCCCC] | 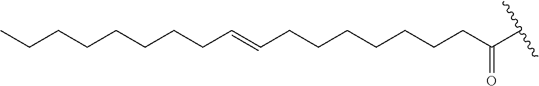 [SMILES: *C(=O)CCCCCCC/C=C/CCCCCCCC] |
| 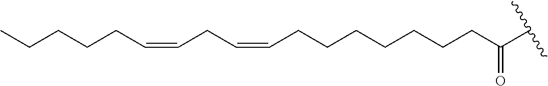 [SMILES: *C(=O)CCCCCCC/C=C\C/C=C\CCCCC] | 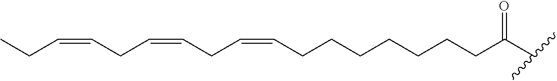 [SMILES: *C(=O)CCCCCCC/C=C\C/C=C\C/C=C\CC] |
| 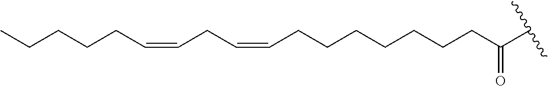 [SMILES: *C(=O)CCCCCCC/C=C\C/C=C\CCCCC] | tBu |
| 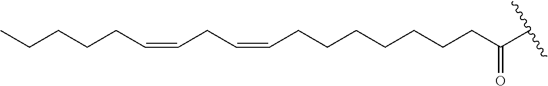 [SMILES: *C(=O)CCCCCCC/C=C\C/C=C\CCCCC] | phenyl |
| 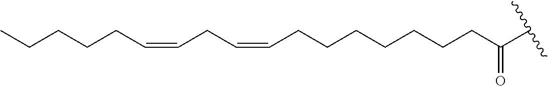 [SMILES: *C(=O)CCCCCCC/C=C\C/C=C\CCCCC] | 4-chlorophenyl |
| 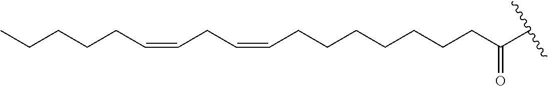 [SMILES: *C(=O)CCCCCCC/C=C\C/C=C\CCCCC] | 3,4-dichlorophenyl |
| 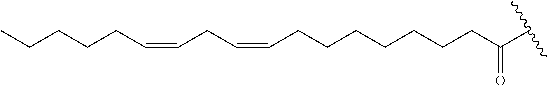 [SMILES: *C(=O)CCCCCCC/C=C\C/C=C\CCCCC] | 4-methylphenyl |
| 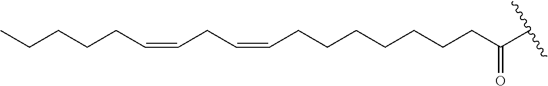 [SMILES: *C(=O)CCCCCCC/C=C\C/C=C\CCCCC] | 4-methoxyphenyl |
| 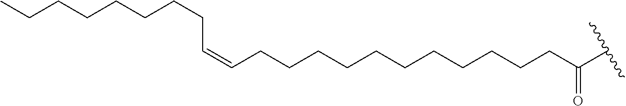 [SMILES: *C(=O)CCCCCCCCCCC/C=C\CCCCCCCC] | Me |
| 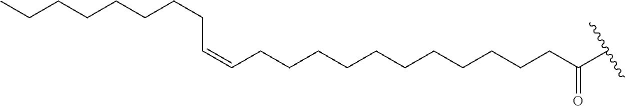 [SMILES: *C(=O)CCCCCCCCCCC/C=C\CCCCCCCC] | —C₇H₁₅ |
| 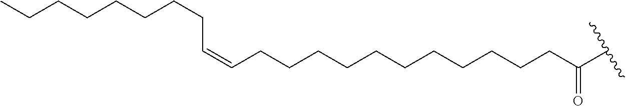 [SMILES: *C(=O)CCCCCCCCCCC/C=C\CCCCCCCC] | —C₉H₁₉ |
| 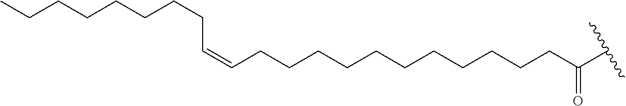 [SMILES: *C(=O)CCCCCCCCCCC/C=C\CCCCCCCC] | —C₁₁H₂₃ |
| 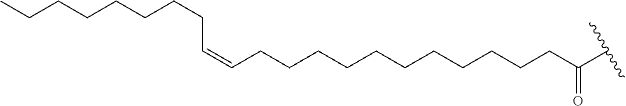 [SMILES: *C(=O)CCCCCCCCCCC/C=C\CCCCCCCC] | —C₁₃H₂₇ |
| 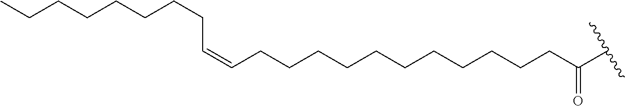 [SMILES: *C(=O)CCCCCCCCCCC/C=C\CCCCCCCC] | —C₁₅H₃₁ |
| 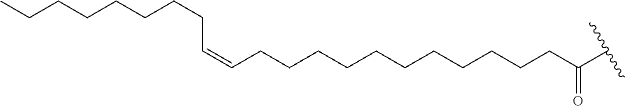 [SMILES: *C(=O)CCCCCCCCCCC/C=C\CCCCCCCC] | —C₁₇H₃₅ |
| 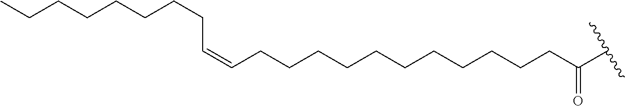 [SMILES: *C(=O)CCCCCCCCCCC/C=C\CCCCCCCC] | 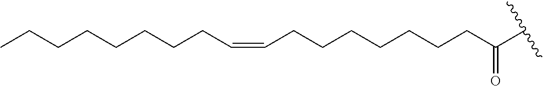 [SMILES: *C(=O)CCCCCCC/C=C\CCCCCCCC] |
| 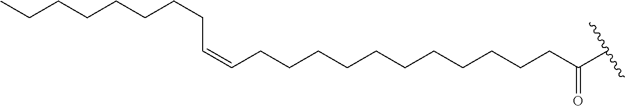 [SMILES: *C(=O)CCCCCCCCCCC/C=C\CCCCCCCC] | 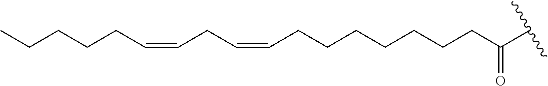 [SMILES: *C(=O)CCCCCCC/C=C\C/C=C\CCCCC] |
| 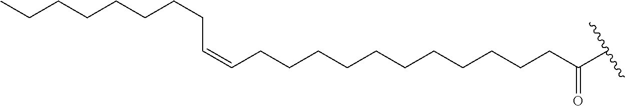 [SMILES: *C(=O)CCCCCCCCCCC/C=C\CCCCCCCC] | 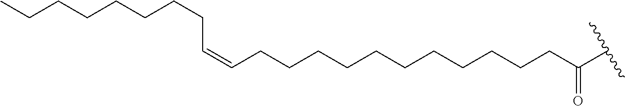 [SMILES: *C(=O)CCCCCCCCCCC/C=C\CCCCCCCC] |
| 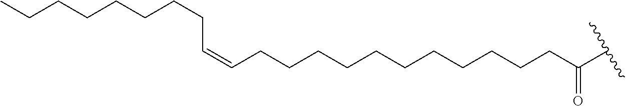 [SMILES: *C(=O)CCCCCCCCCCC/C=C\CCCCCCCC] | 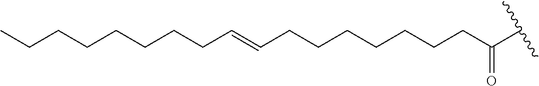 [SMILES: *C(=O)CCCCCCC/C=C/CCCCCCCC] |
| 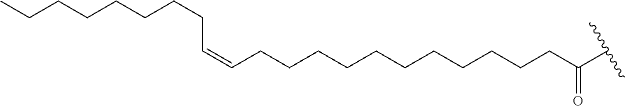 [SMILES: *C(=O)CCCCCCCCCCC/C=C\CCCCCCCC] | 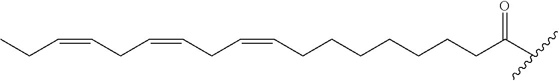 [SMILES: *C(=O)CCCCCCC/C=C\C/C=C\C/C=C\CC] |
| 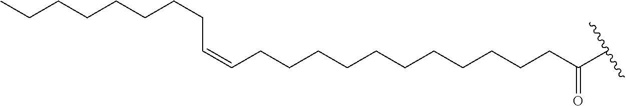 [SMILES: *C(=O)CCCCCCCCCCC/C=C\CCCCCCCC] | tBu |
| 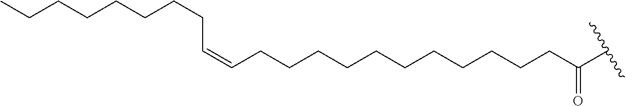 [SMILES: *C(=O)CCCCCCCCCCC/C=C\CCCCCCCC] | phenyl |
| 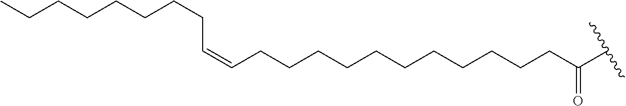 [SMILES: *C(=O)CCCCCCCCCCC/C=C\CCCCCCCC] | 4-chlorophenyl |
| 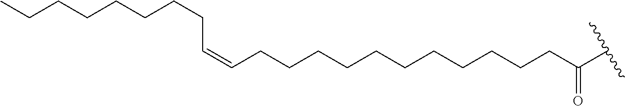 [SMILES: *C(=O)CCCCCCCCCCC/C=C\CCCCCCCC] | 3,4-dichlorophenyl |
| 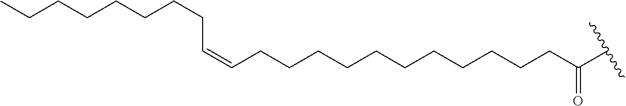 [SMILES: *C(=O)CCCCCCCCCCC/C=C\CCCCCCCC] | 4-methylphenyl |
| 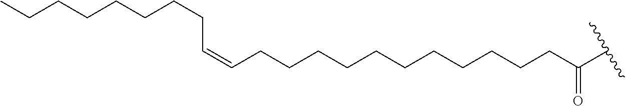 [SMILES: *C(=O)CCCCCCCCCCC/C=C\CCCCCCCC] | 4-methoxyphenyl |
| 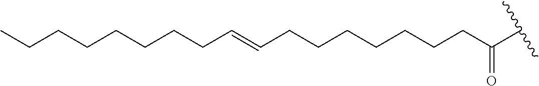 [SMILES: *C(=O)CCCCCCC/C=C/CCCCCCCC] | Me |
| 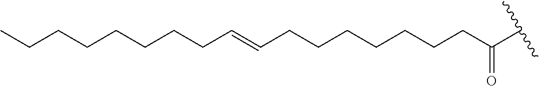 [SMILES: *C(=O)CCCCCCC/C=C/CCCCCCCC] | —C₇H₁₅ |
| 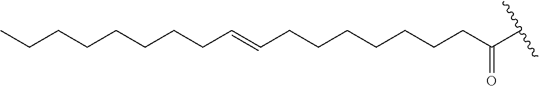 [SMILES: *C(=O)CCCCCCC/C=C/CCCCCCCC] | —C₉H₁₉ |
| 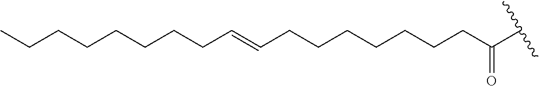 [SMILES: *C(=O)CCCCCCC/C=C/CCCCCCCC] | —C₁₁H₂₃ |
| 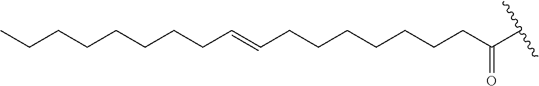 [SMILES: *C(=O)CCCCCCC/C=C/CCCCCCCC] | —C₁₃H₂₇ |
| 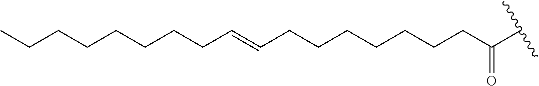 [SMILES: *C(=O)CCCCCCC/C=C/CCCCCCCC] | —C₁₅H₃₁ |
| 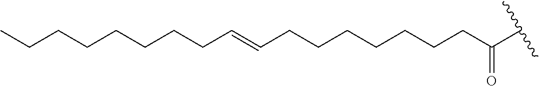 [SMILES: *C(=O)CCCCCCC/C=C/CCCCCCCC] | —C₁₇H₃₅ |
| 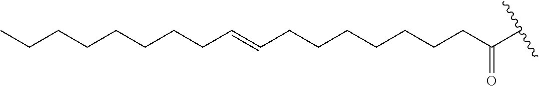 [SMILES: *C(=O)CCCCCCC/C=C/CCCCCCCC] | 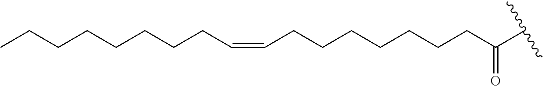 [SMILES: *C(=O)CCCCCCC/C=C\CCCCCCCC] |
| 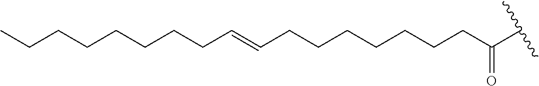 [SMILES: *C(=O)CCCCCCC/C=C/CCCCCCCC] | 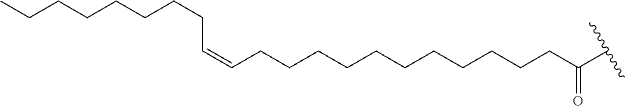 [SMILES: *C(=O)CCCCCCCCCCC/C=C\CCCCCCCC] |
| 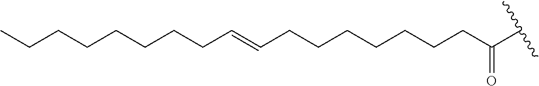 [SMILES: *C(=O)CCCCCCC/C=C/CCCCCCCC] | 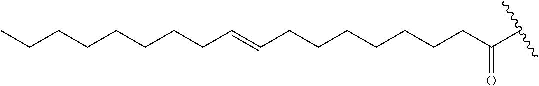 [SMILES: *C(=O)CCCCCCC/C=C/CCCCCCCC] |
| 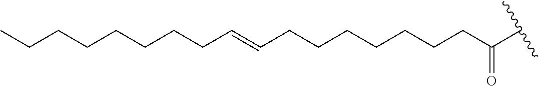 [SMILES: *C(=O)CCCCCCC/C=C/CCCCCCCC] | 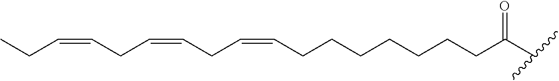 [SMILES: *C(=O)CCCCCCC/C=C\C/C=C\C/C=C\CC] |
| 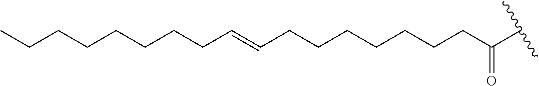 [SMILES: *C(=O)CCCCCCC/C=C/CCCCCCCC] | tBu |
| 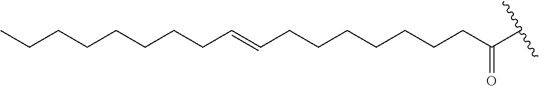 [SMILES: *C(=O)CCCCCCC/C=C/CCCCCCCC] | phenyl |
| 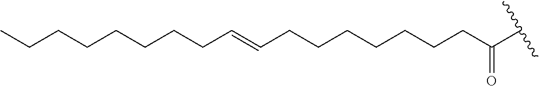 [SMILES: *C(=O)CCCCCCC/C=C/CCCCCCCC] | 4-chlorophenyl |
| 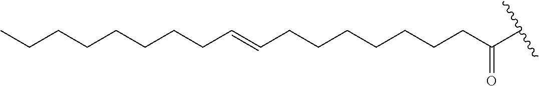 [SMILES: *C(=O)CCCCCCC/C=C/CCCCCCCC] | 3,4-dichlorophenyl |
| 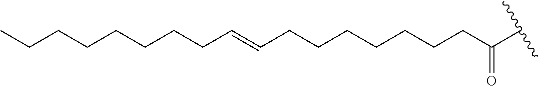 [SMILES: *C(=O)CCCCCCC/C=C/CCCCCCCC] | 4-methylphenyl |
| 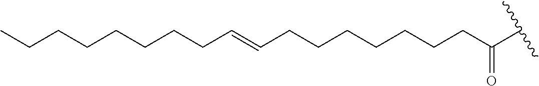 [SMILES: *C(=O)CCCCCCC/C=C/CCCCCCCC] | 4-methoxyphenyl |
| 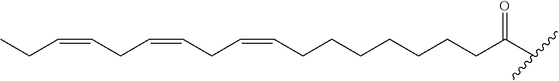 [SMILES: *C(=O)CCCCCCC/C=C\C/C=C\C/C=C\CC] | Me |
| 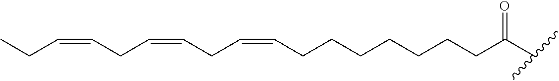 [SMILES: *C(=O)CCCCCCC/C=C\C/C=C\C/C=C\CC] | —C₇H₁₅ |
| 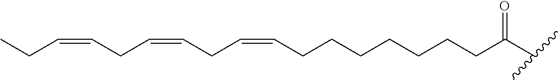 [SMILES: *C(=O)CCCCCCC/C=C\C/C=C\C/C=C\CC] | —C₉H₁₉ |
| 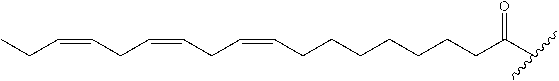 [SMILES: *C(=O)CCCCCCC/C=C\C/C=C\C/C=C\CC] | —C₁₁H₂₃ |
| 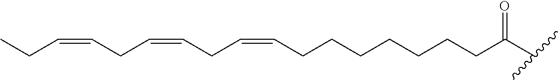 [SMILES: *C(=O)CCCCCCC/C=C\C/C=C\C/C=C\CC] | —C₁₃H₂₇ |
| 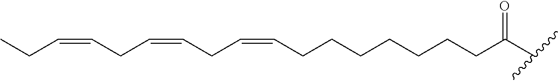 [SMILES: *C(=O)CCCCCCC/C=C\C/C=C\C/C=C\CC] | —C₁₅H₃₁ |
| 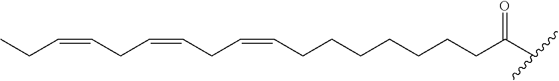 [SMILES: *C(=O)CCCCCCC/C=C\C/C=C\C/C=C\CC] | —C₁₇H₃₅ |
| 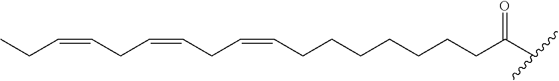 [SMILES: *C(=O)CCCCCCC/C=C\C/C=C\C/C=C\CC] | 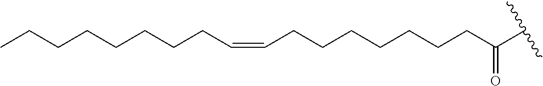 [SMILES: *C(=O)CCCCCCC/C=C\CCCCCCCC] |
| 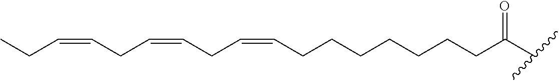 [SMILES: *C(=O)CCCCCCC/C=C\C/C=C\C/C=C\CC] | 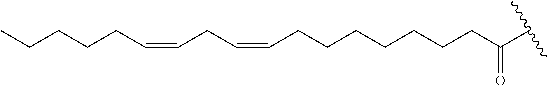 [SMILES: *C(=O)CCCCCCC/C=C\C/C=C\CCCCC] |
| 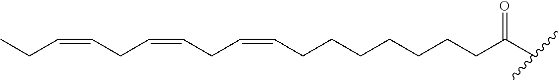 [SMILES: *C(=O)CCCCCCC/C=C\C/C=C\C/C=C\CC] | 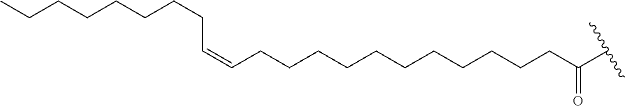 [SMILES: *C(=O)CCCCCCCCCCC/C=C\CCCCCCCC] |
| 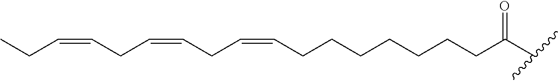 [SMILES: *C(=O)CCCCCCC/C=C\C/C=C\C/C=C\CC] | 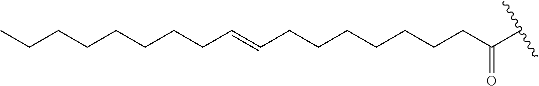 [SMILES: *C(=O)CCCCCCC/C=C/CCCCCCCC] |
| 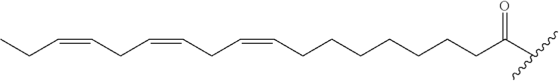 [SMILES: *C(=O)CCCCCCC/C=C\C/C=C\C/C=C\CC] | 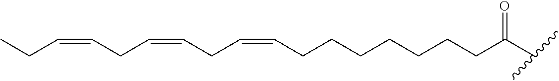 [SMILES: *C(=O)CCCCCCC/C=C\C/C=C\C/C=C\CC] |
| 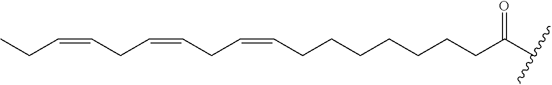 [SMILES: *C(=O)CCCCCCC/C=C\C/C=C\C/C=C\CC] | tBu |
| 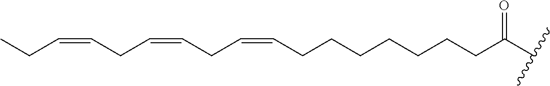 [SMILES: *C(=O)CCCCCCC/C=C\C/C=C\C/C=C\CC] | phenyl |
| 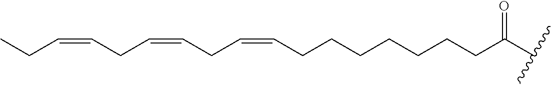 [SMILES: *C(=O)CCCCCCC/C=C\C/C=C\C/C=C\CC] | 4-chlorophenyl |
| 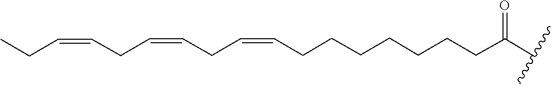 [SMILES: *C(=O)CCCCCCC/C=C\C/C=C\C/C=C\CC] | 3,4-dichlorophenyl |
| 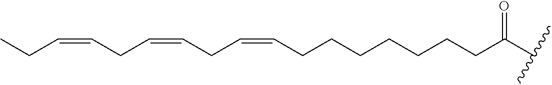 [SMILES: *C(=O)CCCCCCC/C=C\C/C=C\C/C=C\CC] | 4-methylphenyl |
| 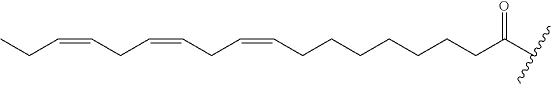 [SMILES: *C(=O)CCCCCCC/C=C\C/C=C\C/C=C\CC] | 4-methoxyphenyl |
| H | 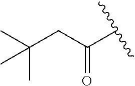 [SMILES: *C(=O)CC(C)(C)C] |
| H | 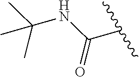 [SMILES: *C(=O)NC(C)(C)C] |
| H | 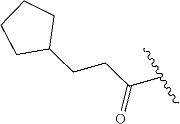 [SMILES: *C(=O)CCC1CCCC1] |
| H | 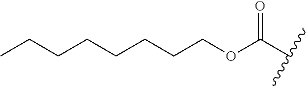 [SMILES: *C(=O)OCCCCCCCC] |
| H | 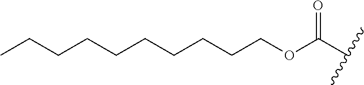 [SMILES: *C(=O)OCCCCCCCCCC] |
| H | 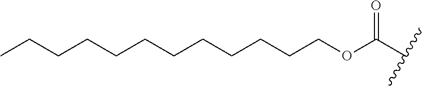 [SMILES: *C(=O)OCCCCCCCCCCCC] |
| H | 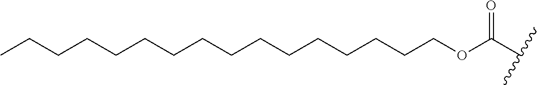 [SMILES: *C(=O)OCCCCCCCCCCCCCCCC] |
| H | 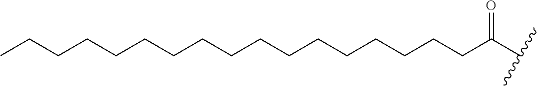 [SMILES: *C(=O)CCCCCCCCCCCCCCCCC] |
| H | 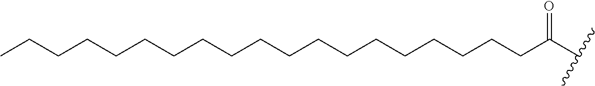 [SMILES: *C(=O)CCCCCCCCCCCCCCCCCCC] |
| H | 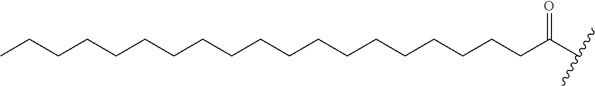 [SMILES: *C(=O)CCCCCCCCCCCCCCCCCCC] |
| H | 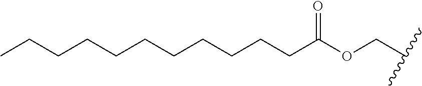 [SMILES: *COC(=O)CCCCCCCCCCC] |
| H | 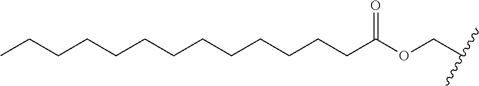 [SMILES: *COC(=O)CCCCCCCCCCCCC] |
| H | 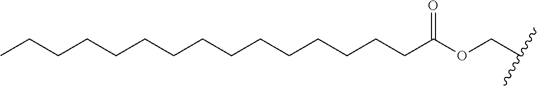 [SMILES: *COC(=O)CCCCCCCCCCCCCCC] |
| H | 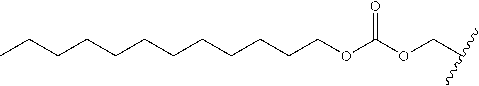 [SMILES: *COC(=O)OCCCCCCCCCCCC] |
| H | 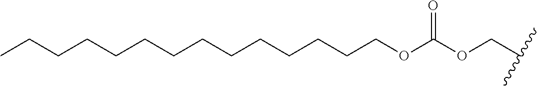 [SMILES: *COC(=O)OCCCCCCCCCCCCCC] |
| H | 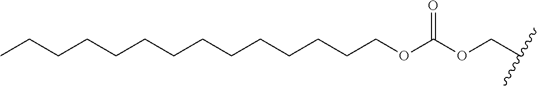 [SMILES: *COC(=O)OCCCCCCCCCCCCCC] |
| Me | 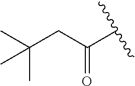 [SMILES: *C(=O)CC(C)(C)C] |
| Me | 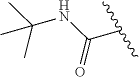 [SMILES: *C(=O)NC(C)(C)C] |
| Me | 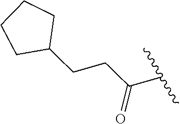 [SMILES: *C(=O)CCC1CCCC1] |
| Me | 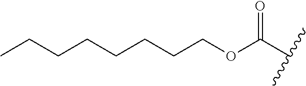 [SMILES: *C(=O)OCCCCCCCC] |
| Me | 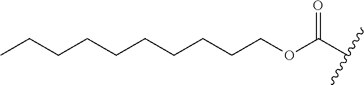 [SMILES: *C(=O)OCCCCCCCCCC] |
| Me | 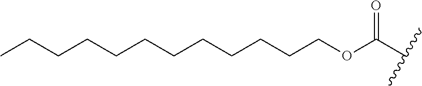 [SMILES: *C(=O)OCCCCCCCCCCCC] |
| Me | 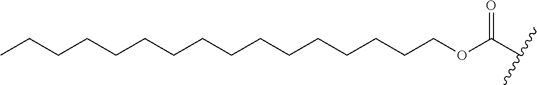 [SMILES: *C(=O)OCCCCCCCCCCCCCCCC] |
| Me | 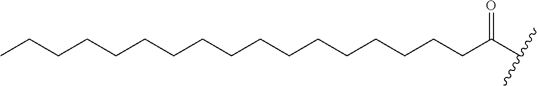 [SMILES: *C(=O)CCCCCCCCCCCCCCCCC] |
| Me | 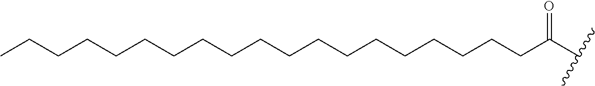 [SMILES: *C(=O)CCCCCCCCCCCCCCCCCCC] |
| Me | 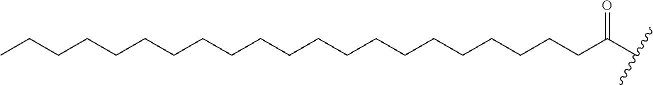 [SMILES: *C(=O)CCCCCCCCCCCCCCCCCCCCC] |
| Me | 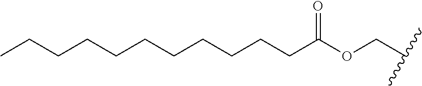 [SMILES: *COC(=O)CCCCCCCCCCC] |
| Me | 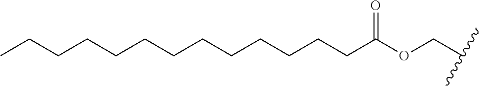 [SMILES: *COC(=O)CCCCCCCCCCCCC] |
| Me | 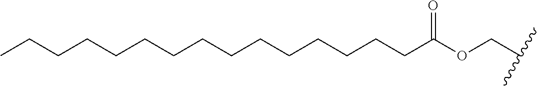 [SMILES: *COC(=O)CCCCCCCCCCCCCCC] |
| Me | 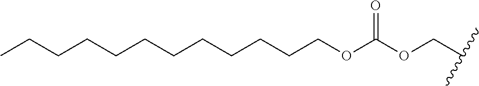 [SMILES: *COC(=O)OCCCCCCCCCCCC] |
| Me | 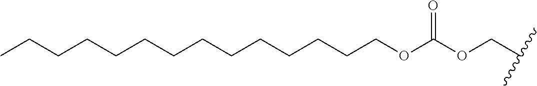 [SMILES: *COC(=O)OCCCCCCCCCCCCCC] |
| Me | 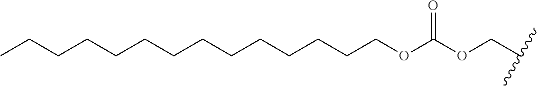 [SMILES: *COC(=O)OCCCCCCCCCCCCCC] |
| —C₇H₁₅ | 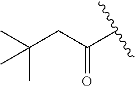 [SMILES: *C(=O)CC(C)(C)C] |
| —C₇H₁₅ | 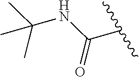 [SMILES: *C(=O)NC(C)(C)C] |
| —C₇H₁₅ | 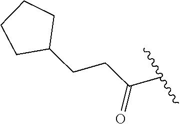 [SMILES: *C(=O)CCC1CCCC1] |
| —C₇H₁₅ | 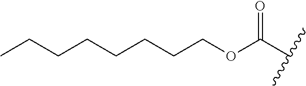 [SMILES: *C(=O)OCCCCCCCC] |
| —C₇H₁₅ | 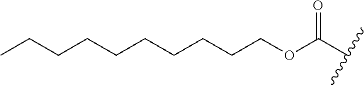 [SMILES: *C(=O)OCCCCCCCCCC] |
| —C₇H₁₅ | 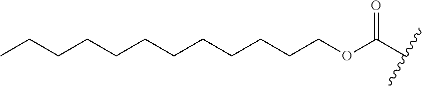 [SMILES: *C(=O)OCCCCCCCCCCCC] |
| —C₇H₁₅ | 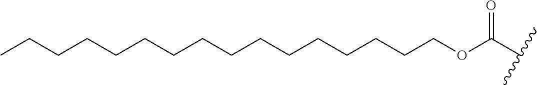 [SMILES: *C(=O)OCCCCCCCCCCCCCCCC] |
| —C₇H₁₅ | 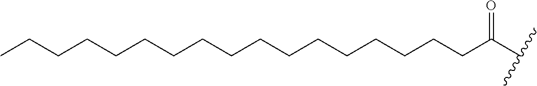 [SMILES: *C(=O)CCCCCCCCCCCCCCCCC] |
| —C₇H₁₅ | 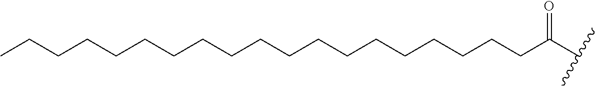 [SMILES: *C(=O)CCCCCCCCCCCCCCCCCCC] |
| —C₇H₁₅ | 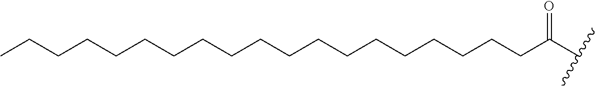 [SMILES: *C(=O)CCCCCCCCCCCCCCCCCCC] |
| —C₇H₁₅ | 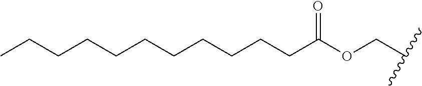 [SMILES: *COC(=O)CCCCCCCCCCC] |
| —C₇H₁₅ | 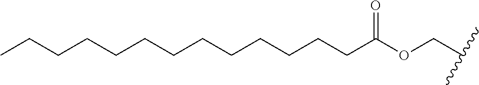 [SMILES: *COC(=O)CCCCCCCCCCCCC] |
| —C₇H₁₅ | 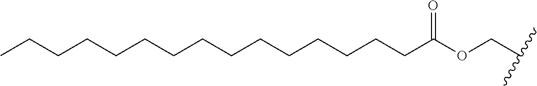 [SMILES: *COC(=O)CCCCCCCCCCCCCCC] |
| —C₇H₁₅ | 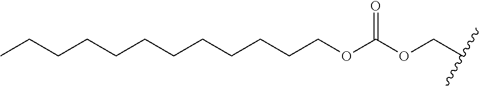 [SMILES: *COC(=O)OCCCCCCCCCCCC] |
| —C₇H₁₅ | 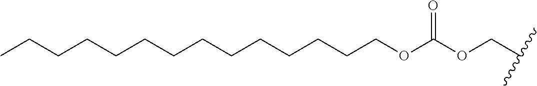 [SMILES: *COC(=O)OCCCCCCCCCCCCCC] |
| —C₇H₁₅ | 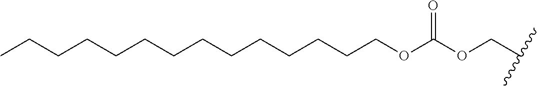 [SMILES: *COC(=O)OCCCCCCCCCCCCCC] |
| —C₉H₁₉ | 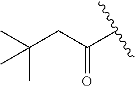 [SMILES: *C(=O)CC(C)(C)C] |
| —C₉H₁₉ | 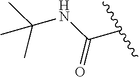 [SMILES: *C(=O)NC(C)(C)C] |
| —C₉H₁₉ | 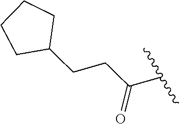 [SMILES: *C(=O)CCC1CCCC1] |
| —C₉H₁₉ | 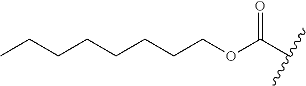 [SMILES: *C(=O)OCCCCCCCC] |
| —C₉H₁₉ | 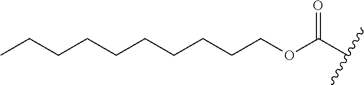 [SMILES: *C(=O)OCCCCCCCCCC] |
| —C₉H₁₉ | 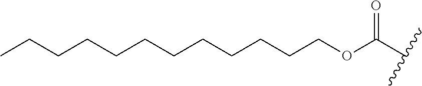 [SMILES: *C(=O)OCCCCCCCCCCCC] |
| —C₉H₁₉ | 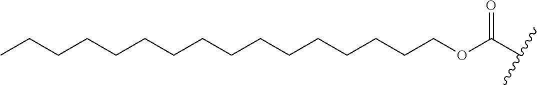 [SMILES: *C(=O)OCCCCCCCCCCCCCCCC] |
| —C₉H₁₉ | 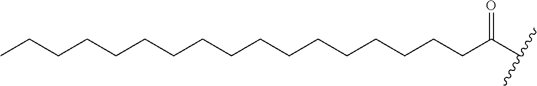 [SMILES: *C(=O)CCCCCCCCCCCCCCCCC] |
| —C₉H₁₉ | 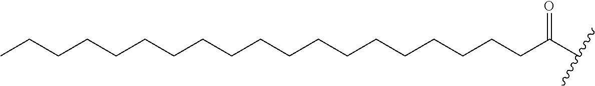 [SMILES: *C(=O)CCCCCCCCCCCCCCCCCCC] |
| —C₉H₁₉ | 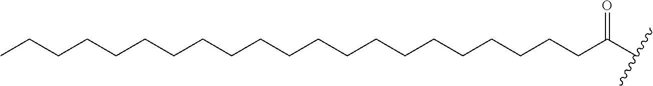 [SMILES: *C(=O)CCCCCCCCCCCCCCCCCCCCC] |
| —C₉H₁₉ | 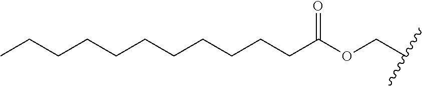 [SMILES: *COC(=O)CCCCCCCCCCC] |
| —C₉H₁₉ | 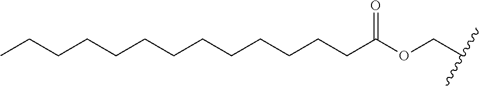 [SMILES: *COC(=O)CCCCCCCCCCCCC] |
| —C₉H₁₉ | 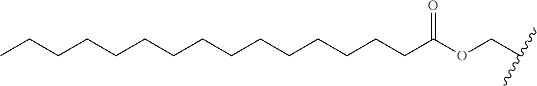 [SMILES: *COC(=O)CCCCCCCCCCCCCCC] |
| —C₉H₁₉ | 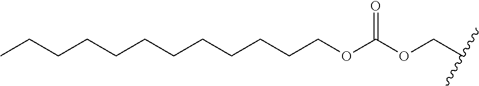 [SMILES: *COC(=O)OCCCCCCCCCCCC] |
| —C₉H₁₉ | 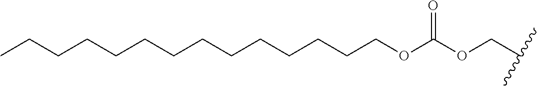 [SMILES: *COC(=O)OCCCCCCCCCCCCCC] |
| —C₉H₁₉ | 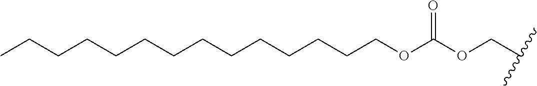 [SMILES: *COC(=O)OCCCCCCCCCCCCCC] |
| —C₁₁H₂₃ | 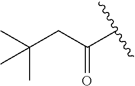 [SMILES: *C(=O)CC(C)(C)C] |
| —C₁₁H₂₃ | 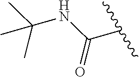 [SMILES: *C(=O)NC(C)(C)C] |
| —C₁₁H₂₃ | 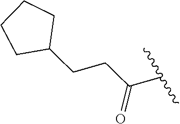 [SMILES: *C(=O)CCC1CCCC1] |
| —C₁₁H₂₃ | 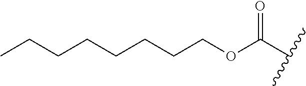 [SMILES: *C(=O)OCCCCCCCC] |
| —C₁₁H₂₃ | 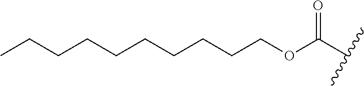 [SMILES: *C(=O)OCCCCCCCCCC] |
| —C₁₁H₂₃ | 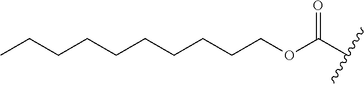 [SMILES: *C(=O)OCCCCCCCCCC] |
| —C₁₁H₂₃ | 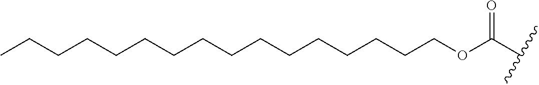 [SMILES: *C(=O)OCCCCCCCCCCCCCCCC] |
| —C₁₁H₂₃ | 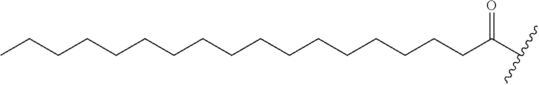 [SMILES: *C(=O)CCCCCCCCCCCCCCCCC] |
| —C₁₁H₂₃ | 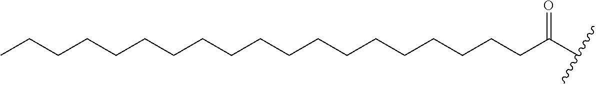 [SMILES: *C(=O)CCCCCCCCCCCCCCCCCCC] |
| —C₁₁H₂₃ | 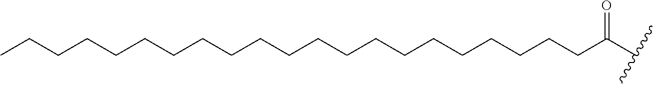 [SMILES: *C(=O)CCCCCCCCCCCCCCCCCCCCC] |
| —C₁₁H₂₃ | 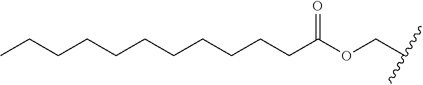 [SMILES: *COC(=O)CCCCCCCCCCC] |
| —C₁₁H₂₃ | 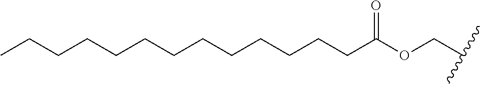 [SMILES: *COC(=O)CCCCCCCCCCCCC] |
| —C₁₁H₂₃ | 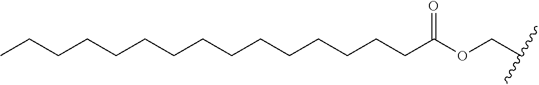 [SMILES: *COC(=O)CCCCCCCCCCCCCCC] |
| —C₁₁H₂₃ | 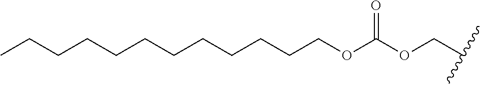 [SMILES: *COC(=O)OCCCCCCCCCCCC] |
| —C₁₁H₂₃ | 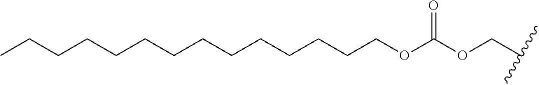 [SMILES: *COC(=O)OCCCCCCCCCCCCCC] |
| —C₁₁H₂₃ | 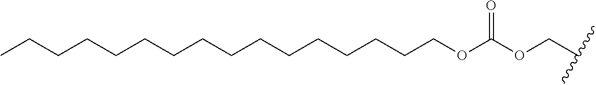 [SMILES: *COC(=O)OCCCCCCCCCCCCCCCC] |
| —C₁₃H₂₇ | 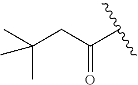 [SMILES: *C(=O)CC(C)(C)C] |
| —C₁₃H₂₇ | 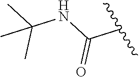 [SMILES: *C(=O)NC(C)(C)C] |
| —C₁₃H₂₇ | 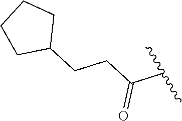 [SMILES: *C(=O)CCC1CCCC1] |
| —C₁₃H₂₇ | 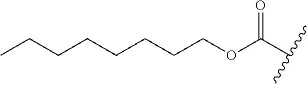 [SMILES: *C(=O)OCCCCCCCC] |
| —C₁₃H₂₇ | 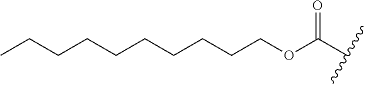 [SMILES: *C(=O)OCCCCCCCCCC] |
| —C₁₃H₂₇ | 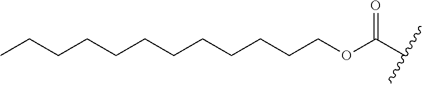 [SMILES: *C(=O)OCCCCCCCCCCCC] |
| —C₁₃H₂₇ | 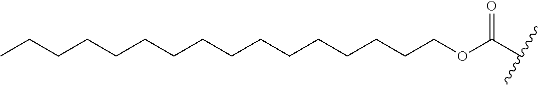 [SMILES: *C(=O)OCCCCCCCCCCCCCCCC] |
| —C₁₃H₂₇ | 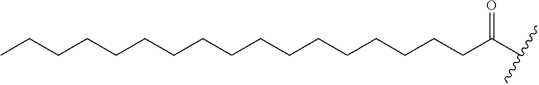 [SMILES: *C(=O)CCCCCCCCCCCCCCCCC] |
| —C₁₃H₂₇ | 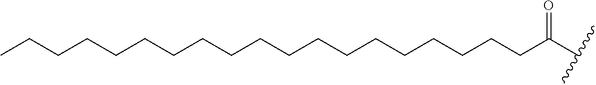 [SMILES: *C(=O)CCCCCCCCCCCCCCCCCCC] |
| —C₁₃H₂₇ | 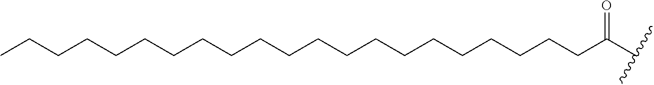 [SMILES: *C(=O)CCCCCCCCCCCCCCCCCCCCC] |
| —C₁₃H₂₇ | 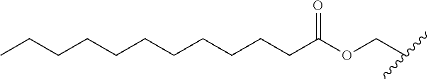 [SMILES: *COC(=O)CCCCCCCCCCC] |
| —C₁₃H₂₇ | 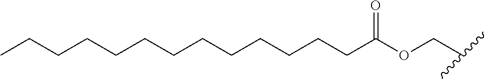 [SMILES: *COC(=O)CCCCCCCCCCCCC] |
| —C₁₃H₂₇ | 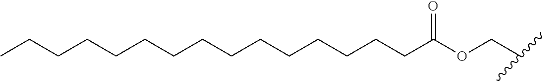 [SMILES: *COC(=O)CCCCCCCCCCCCCCC] |
| —C₁₃H₂₇ | 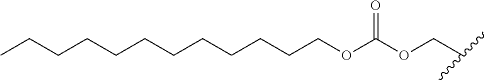 [SMILES: *COC(=O)OCCCCCCCCCCCC] |
| —C₁₃H₂₇ | 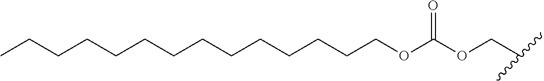 [SMILES: *COC(=O)OCCCCCCCCCCCCCC] |
| —C₁₃H₂₇ | 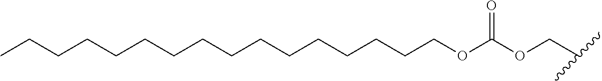 [SMILES: *COC(=O)OCCCCCCCCCCCCCCCC] |
| —C₁₅H₃₁ | 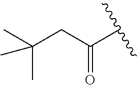 [SMILES: *C(=O)CC(C)(C)C] |
| —C₁₅H₃₁ | 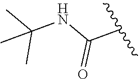 [SMILES: *C(=O)NC(C)(C)C] |
| —C₁₅H₃₁ | 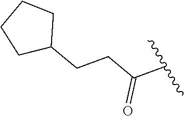 [SMILES: *C(=O)CCC1CCCC1] |
| —C₁₅H₃₁ | 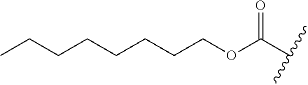 [SMILES: *C(=O)OCCCCCCCC] |
| —C₁₅H₃₁ | 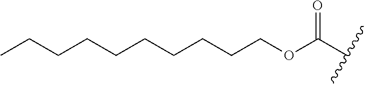 [SMILES: *C(=O)OCCCCCCCCCC] |
| —C₁₅H₃₁ | 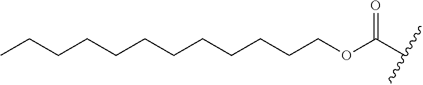 [SMILES: *C(=O)OCCCCCCCCCCCC] |
| —C₁₅H₃₁ | 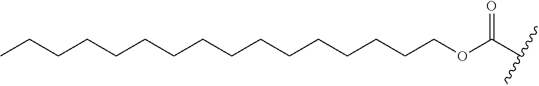 [SMILES: *C(=O)OCCCCCCCCCCCCCCCC] |
| —C₁₅H₃₁ | 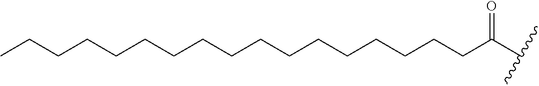 [SMILES: *C(=O)CCCCCCCCCCCCCCCCC] |
| —C₁₅H₃₁ | 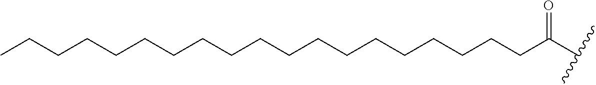 [SMILES: *C(=O)CCCCCCCCCCCCCCCCCCC] |
| —C₁₅H₃₁ | 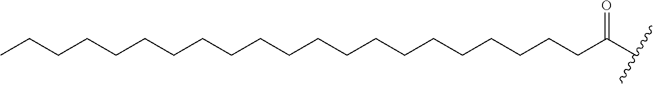 [SMILES: *C(=O)CCCCCCCCCCCCCCCCCCCCC] |
| —C₁₅H₃₁ | 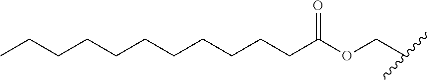 [SMILES: *COC(=O)CCCCCCCCCCC] |
| —C₁₅H₃₁ | 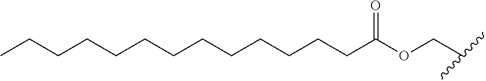 [SMILES: *COC(=O)CCCCCCCCCCCCC] |
| —C₁₅H₃₁ | 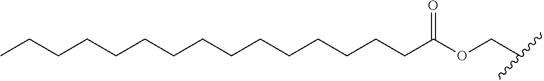 [SMILES: *COC(=O)CCCCCCCCCCCCCCC] |
| —C₁₅H₃₁ | 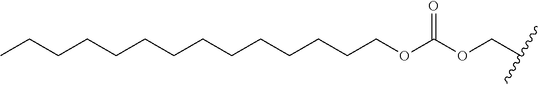 [SMILES: *COC(=O)OCCCCCCCCCCCCCC] |
| —C₁₅H₃₁ | 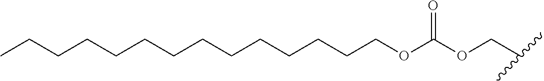 [SMILES: *COC(=O)OCCCCCCCCCCCCCC] |
| —C₁₅H₃₁ | 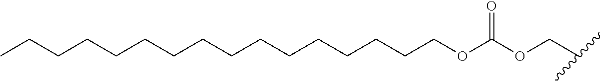 [SMILES: *COC(=O)OCCCCCCCCCCCCCCCC] |
| —C₁₇H₃₅ | 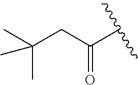 [SMILES: *C(=O)CC(C)(C)C] |
| —C₁₇H₃₅ | 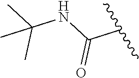 [SMILES: *C(=O)NC(C)(C)C] |
| —C₁₇H₃₅ | 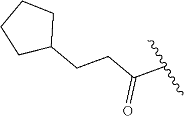 [SMILES: *C(=O)CCC1CCCC1] |
| —C₁₇H₃₅ | 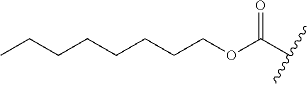 [SMILES: *C(=O)OCCCCCCCC] |
| —C₁₇H₃₅ | 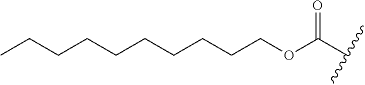 [SMILES: *C(=O)OCCCCCCCCCC] |
| —C₁₇H₃₅ | 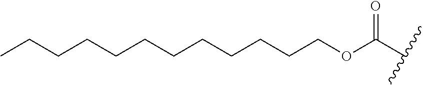 [SMILES: *C(=O)OCCCCCCCCCCCC] |
| —C₁₇H₃₅ | 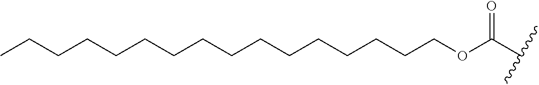 [SMILES: *C(=O)OCCCCCCCCCCCCCCCC] |
| —C₁₇H₃₅ | 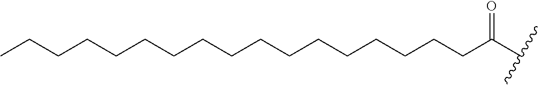 [SMILES: *C(=O)CCCCCCCCCCCCCCCCC] |
| —C₁₇H₃₅ | 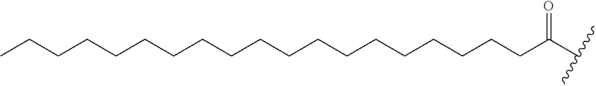 [SMILES: *C(=O)CCCCCCCCCCCCCCCCCCC] |
| —C₁₇H₃₅ | 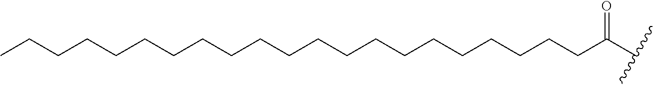 [SMILES: *C(=O)CCCCCCCCCCCCCCCCCCCCC] |
| —C₁₇H₃₅ | 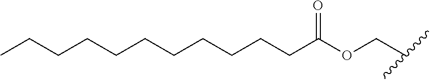 [SMILES: *COC(=O)CCCCCCCCCCC] |
| —C₁₇H₃₅ | 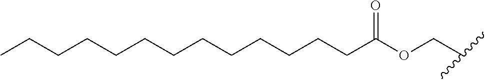 [SMILES: *COC(=O)CCCCCCCCCCCCC] |
| —C₁₇H₃₅ | 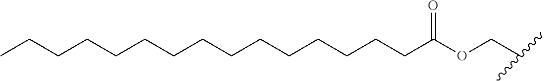 [SMILES: *COC(=O)CCCCCCCCCCCCCCC] |
| —C₁₇H₃₅ | 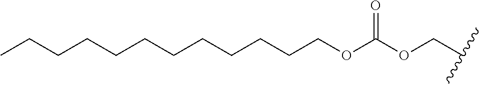 [SMILES: *COC(=O)OCCCCCCCCCCCC] |
| —C₁₇H₃₅ | 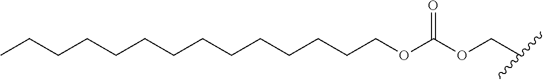 [SMILES: *COC(=O)OCCCCCCCCCCCCCC] |
| —C₁₇H₃₅ | 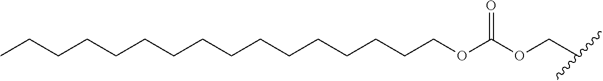 [SMILES: *COC(=O)OCCCCCCCCCCCCCCCC] |
| 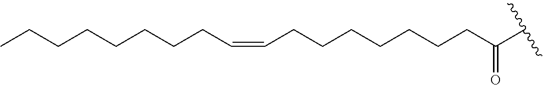 [SMILES: *C(=O)CCCCCCC/C=C\CCCCCCCC] | 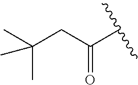 [SMILES: *C(=O)CC(C)(C)C] |
| 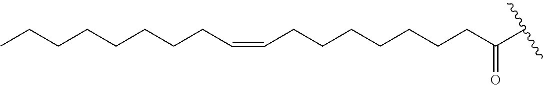 [SMILES: *C(=O)CCCCCCC/C=C\CCCCCCCC] | 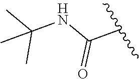 [SMILES: *C(=O)NC(C)(C)C] |
| 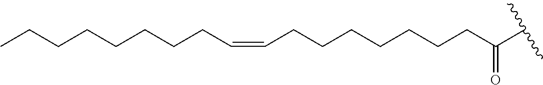 [SMILES: *C(=O)CCCCCCC/C=C\CCCCCCCC] | 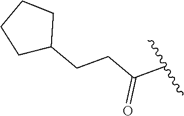 [SMILES: *C(=O)CCC1CCCC1] |
| 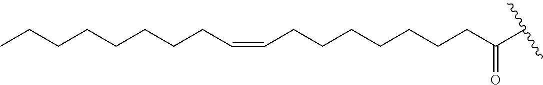 [SMILES: *C(=O)CCCCCCC/C=C\CCCCCCCC] | 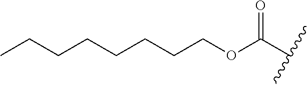 [SMILES: *C(=O)OCCCCCCCC] |
| 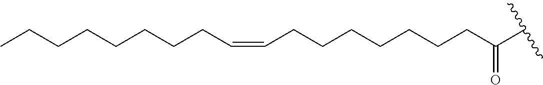 [SMILES: *C(=O)CCCCCCC/C=C\CCCCCCCC] | 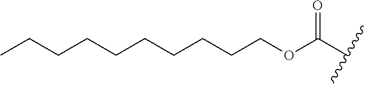 [SMILES: *C(=O)OCCCCCCCCCC] |
| 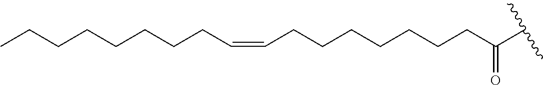 [SMILES: *C(=O)CCCCCCC/C=C\CCCCCCCC] | 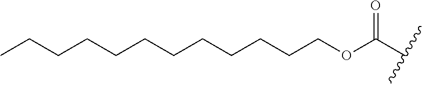 [SMILES: *C(=O)OCCCCCCCCCCCC] |
| 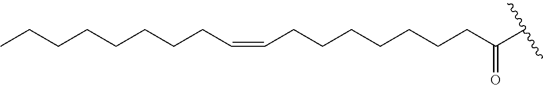 [SMILES: *C(=O)CCCCCCC/C=C\CCCCCCCC] | 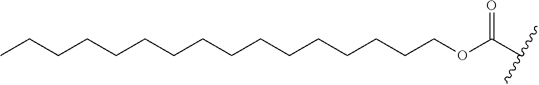 [SMILES: *C(=O)OCCCCCCCCCCCCCCCC] |
| 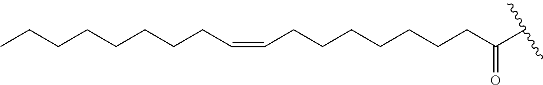 [SMILES: *C(=O)CCCCCCC/C=C\CCCCCCCC] | 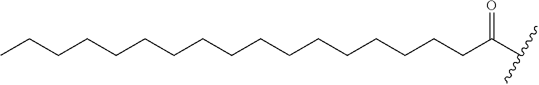 [SMILES: *C(=O)CCCCCCCCCCCCCCCCC] |
| 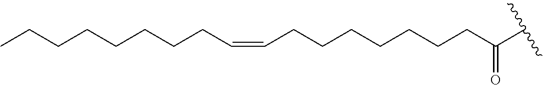 [SMILES: *C(=O)CCCCCCC/C=C\CCCCCCCC] | 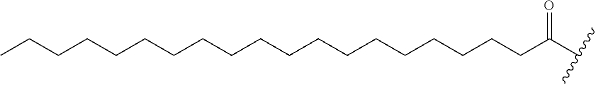 [SMILES: *C(=O)CCCCCCCCCCCCCCCCCCC] |
| 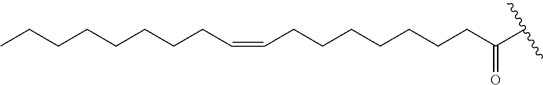 [SMILES: *C(=O)CCCCCCC/C=C\CCCCCCCC] | 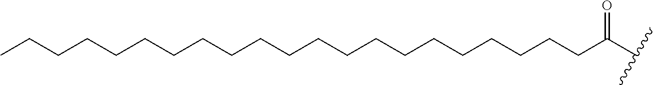 [SMILES: *C(=O)CCCCCCCCCCCCCCCCCCCCC] |
| 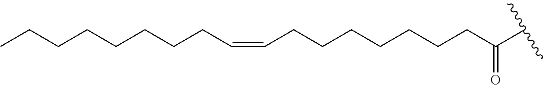 [SMILES: *C(=O)CCCCCCC/C=C\CCCCCCCC] | 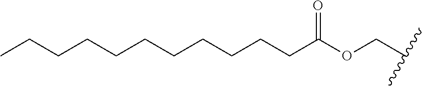 [SMILES: *COC(=O)CCCCCCCCCCC] |
| 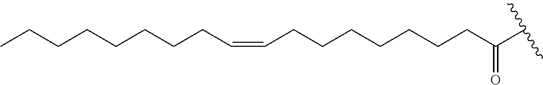 [SMILES: *C(=O)CCCCCCC/C=C\CCCCCCCC] | 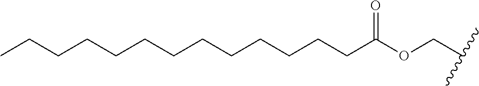 [SMILES: *COC(=O)CCCCCCCCCCCCC] |
| 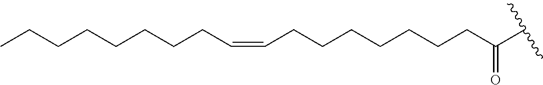 [SMILES: *C(=O)CCCCCCC/C=C\CCCCCCCC] | 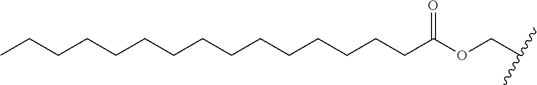 [SMILES: *COC(=O)CCCCCCCCCCCCCCC] |
| 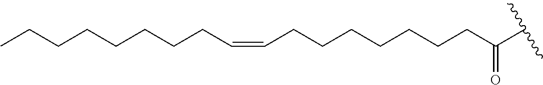 [SMILES: *C(=O)CCCCCCC/C=C\CCCCCCCC] | 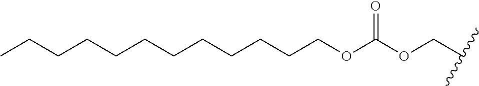 [SMILES: *COC(=O)OCCCCCCCCCCCC] |
| 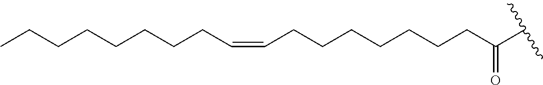 [SMILES: *C(=O)CCCCCCC/C=C\CCCCCCCC] | 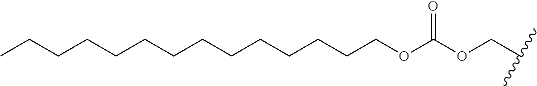 [SMILES: *COC(=O)OCCCCCCCCCCCCCC] |
| 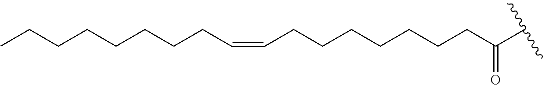 [SMILES: *C(=O)CCCCCCC/C=C\CCCCCCCC] | 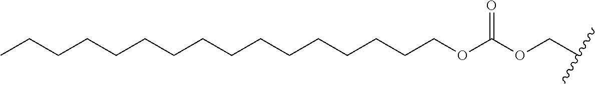 [SMILES: *COC(=O)OCCCCCCCCCCCCCCCC] |
| 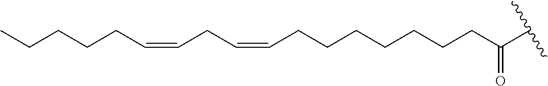 [SMILES: *C(=O)CCCCCCC/C=C\C/C=C\CCCCC] | 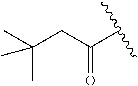 [SMILES: *C(=O)CC(C)(C)C] |
| 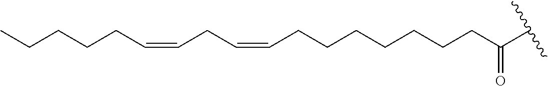 [SMILES: *C(=O)CCCCCCC/C=C\C/C=C\CCCCC] | 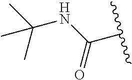 [SMILES: *C(=O)NC(C)(C)C] |
| 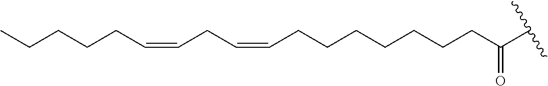 [SMILES: *C(=O)CCCCCCC/C=C\C/C=C\CCCCC] | 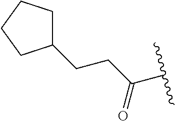 [SMILES: *C(=O)CCC1CCCC1] |
| 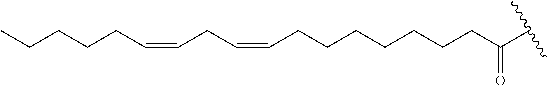 [SMILES: *C(=O)CCCCCCC/C=C\C/C=C\CCCCC] | 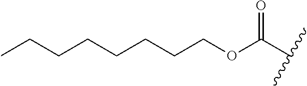 [SMILES: *C(=O)OCCCCCCCC] |
| 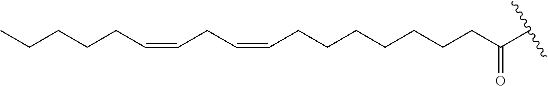 [SMILES: *C(=O)CCCCCCC/C=C\C/C=C\CCCCC] | 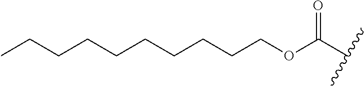 [SMILES: *C(=O)OCCCCCCCCCC] |
| 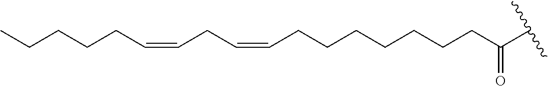 [SMILES: *C(=O)CCCCCCC/C=C\C/C=C\CCCCC] | 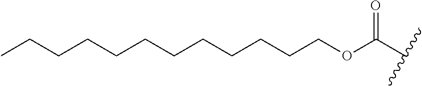 [SMILES: *C(=O)OCCCCCCCCCCCC] |
| 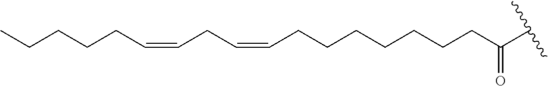 [SMILES: *C(=O)CCCCCCC/C=C\C/C=C\CCCCC] | 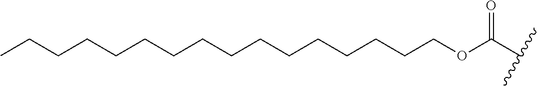 [SMILES: *C(=O)OCCCCCCCCCCCCCCCC] |
| 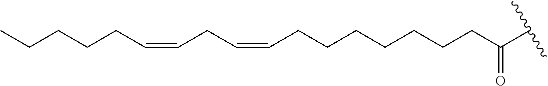 [SMILES: *C(=O)CCCCCCC/C=C\C/C=C\CCCCC] | 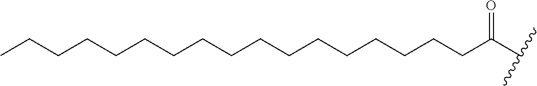 [SMILES: *C(=O)CCCCCCCCCCCCCCCCC] |
| 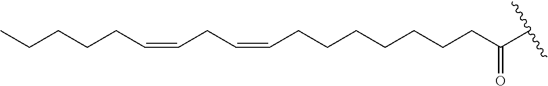 [SMILES: *C(=O)CCCCCCC/C=C\C/C=C\CCCCC] | 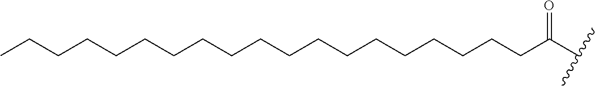 [SMILES: *C(=O)CCCCCCCCCCCCCCCCCCC] |
| 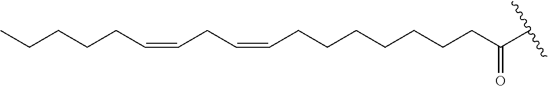 [SMILES: *C(=O)CCCCCCC/C=C\C/C=C\CCCCC] | 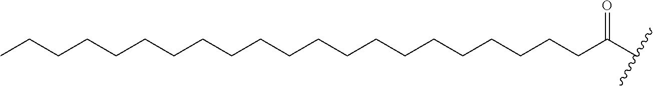 [SMILES: *C(=O)CCCCCCCCCCCCCCCCCCCCC] |
| 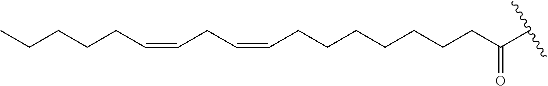 [SMILES: *C(=O)CCCCCCC/C=C\C/C=C\CCCCC] | 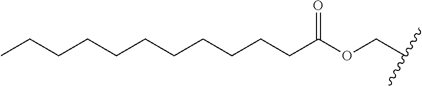 [SMILES: *COC(=O)CCCCCCCCCCC] |
| 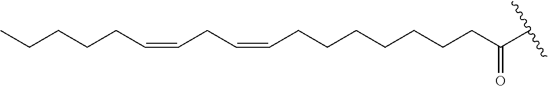 [SMILES: *C(=O)CCCCCCC/C=C\C/C=C\CCCCC] | 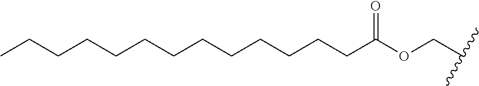 [SMILES: *COC(=O)CCCCCCCCCCCCC] |
| 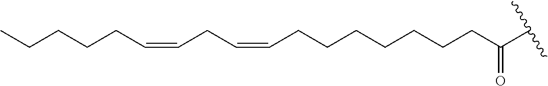 [SMILES: *C(=O)CCCCCCC/C=C\C/C=C\CCCCC] | 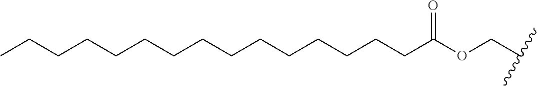 [SMILES: *COC(=O)CCCCCCCCCCCCCCC] |
| 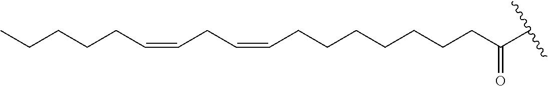 [SMILES: *C(=O)CCCCCCC/C=C\C/C=C\CCCCC] | 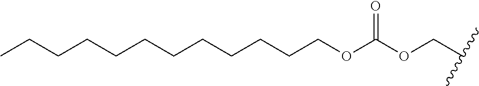 [SMILES: *COC(=O)OCCCCCCCCCCCC] |
| 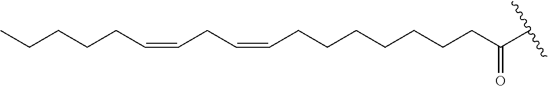 [SMILES: *C(=O)CCCCCCC/C=C\C/C=C\CCCCC] | 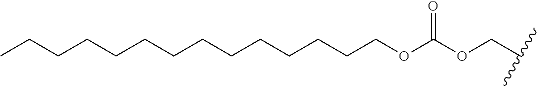 [SMILES: *COC(=O)OCCCCCCCCCCCCCC] |
| 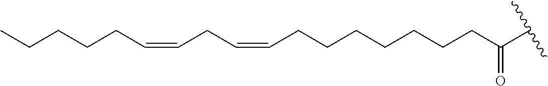 [SMILES: *C(=O)CCCCCCC/C=C\C/C=C\CCCCC] | 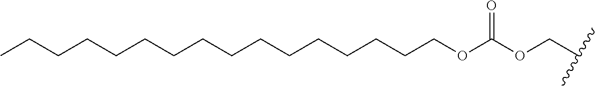 [SMILES: *COC(=O)OCCCCCCCCCCCCCCCC] |
| 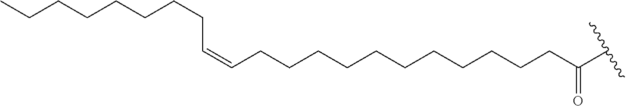 [SMILES: *C(=O)CCCCCCCCCCC/C=C\CCCCCCCC] | 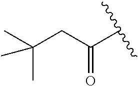 [SMILES: *C(=O)CC(C)(C)C] |
| 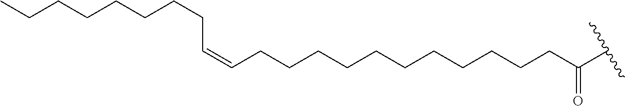 [SMILES: *C(=O)CCCCCCCCCCC/C=C\CCCCCCCC] | 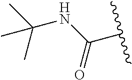 [SMILES: *C(=O)NC(C)(C)C] |
| 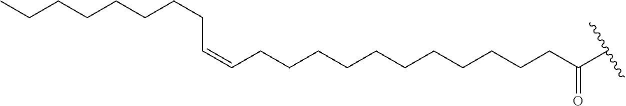 [SMILES: *C(=O)CCCCCCCCCCC/C=C\CCCCCCCC] | 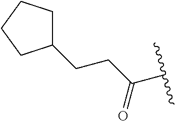 [SMILES: *C(=O)CCC1CCCC1] |
| 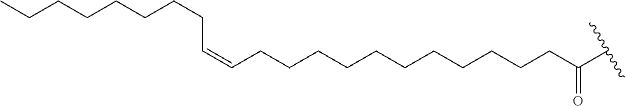 [SMILES: *C(=O)CCCCCCCCCCC/C=C\CCCCCCCC] | 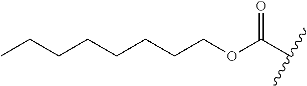 [SMILES: *C(=O)OCCCCCCCC] |
| 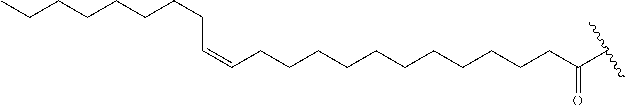 [SMILES: *C(=O)CCCCCCCCCCC/C=C\CCCCCCCC] | 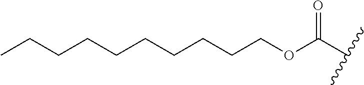 [SMILES: *C(=O)OCCCCCCCCCC] |
| 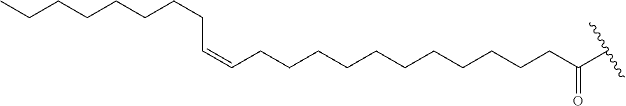 [SMILES: *C(=O)CCCCCCCCCCC/C=C\CCCCCCCC] | 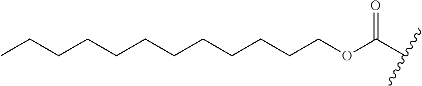 [SMILES: *C(=O)OCCCCCCCCCCCC] |
| 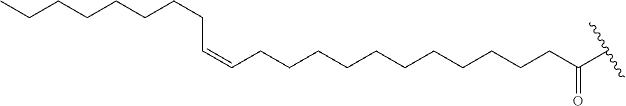 [SMILES: *C(=O)CCCCCCCCCCC/C=C\CCCCCCCC] | 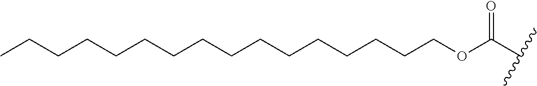 [SMILES: *C(=O)OCCCCCCCCCCCCCCCC] |
| 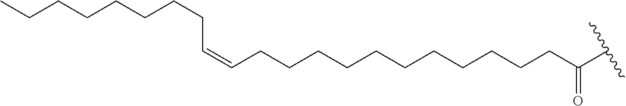 [SMILES: *C(=O)CCCCCCCCCCC/C=C\CCCCCCCC] | 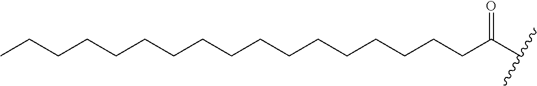 [SMILES: *C(=O)CCCCCCCCCCCCCCCCC] |
| 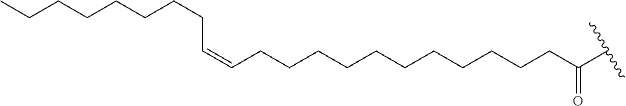 [SMILES: *C(=O)CCCCCCCCCCC/C=C\CCCCCCCC] | 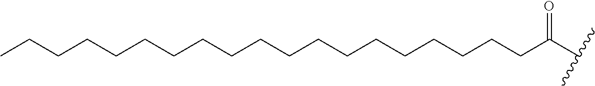 [SMILES: *C(=O)CCCCCCCCCCCCCCCCCCC] |
| 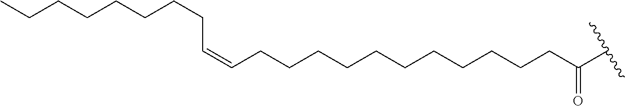 [SMILES: *C(=O)CCCCCCCCCCC/C=C\CCCCCCCC] | 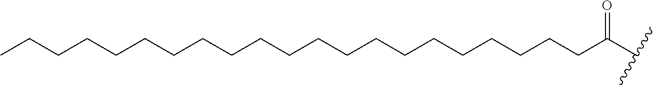 [SMILES: *C(=O)CCCCCCCCCCCCCCCCCCCCC] |
| 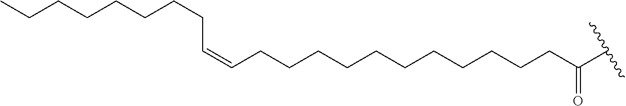 [SMILES: *C(=O)CCCCCCCCCCC/C=C\CCCCCCCC] | 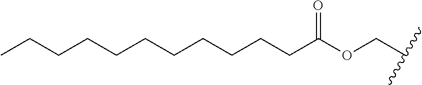 [SMILES: *COC(=O)CCCCCCCCCCC] |
| 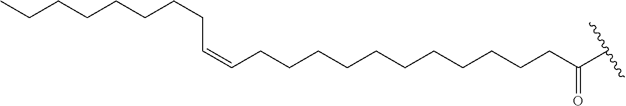 [SMILES: *C(=O)CCCCCCCCCCC/C=C\CCCCCCCC] | 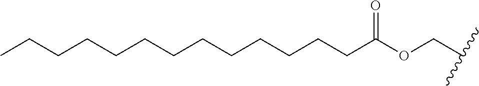 [SMILES: *COC(=O)CCCCCCCCCCCCC] |
| 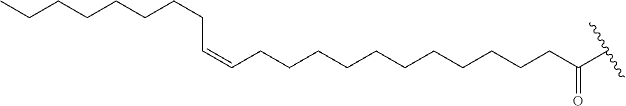 [SMILES: *C(=O)CCCCCCCCCCC/C=C\CCCCCCCC] | 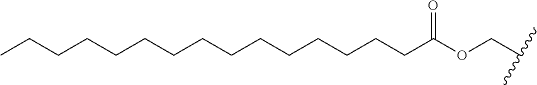 [SMILES: *COC(=O)CCCCCCCCCCCCCCC] |
| 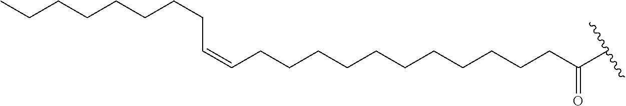 [SMILES: *C(=O)CCCCCCCCCCC/C=C\CCCCCCCC] | 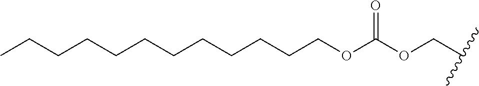 [SMILES: *COC(=O)OCCCCCCCCCCCC] |
| 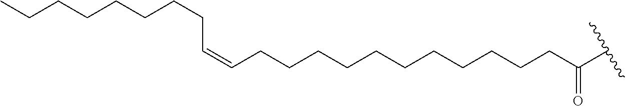 [SMILES: *C(=O)CCCCCCCCCCC/C=C\CCCCCCCC] | 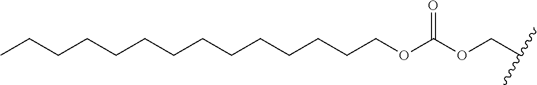 [SMILES: *COC(=O)OCCCCCCCCCCCCCC] |
| 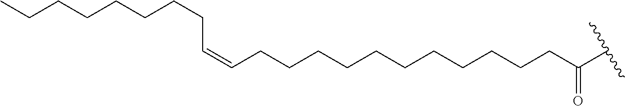 [SMILES: *C(=O)CCCCCCCCCCC/C=C\CCCCCCCC] | 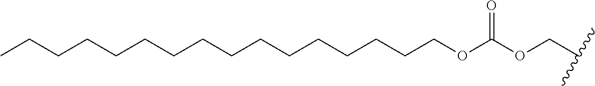 [SMILES: *COC(=O)OCCCCCCCCCCCCCCCC] |
| 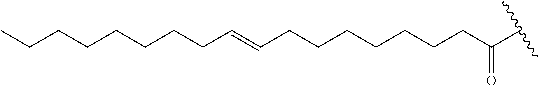 [SMILES: *C(=O)CCCCCCC/C=C/CCCCCCCC] | 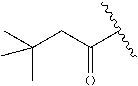 [SMILES: *C(=O)CC(C)(C)C] |
| 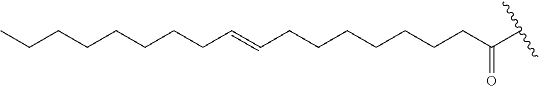 [SMILES: *C(=O)CCCCCCC/C=C/CCCCCCCC] | 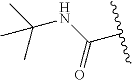 [SMILES: *C(=O)NC(C)(C)C] |
| 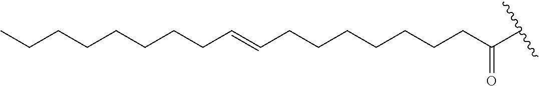 [SMILES: *C(=O)CCCCCCC/C=C/CCCCCCCC] | 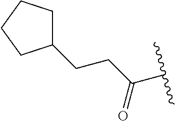 [SMILES: *C(=O)CCC1CCCC1] |
| 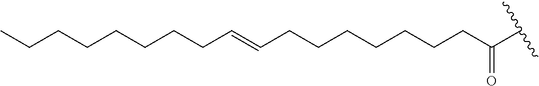 [SMILES: *C(=O)CCCCCCC/C=C/CCCCCCCC] | 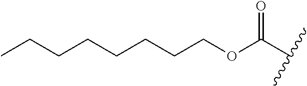 [SMILES: *C(=O)OCCCCCCCC] |
| 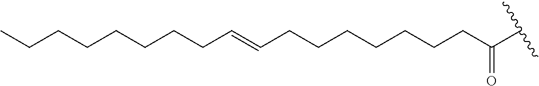 [SMILES: *C(=O)CCCCCCC/C=C/CCCCCCCC] | 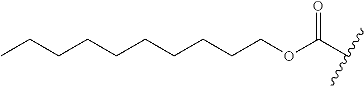 [SMILES: *C(=O)OCCCCCCCCCC] |
| 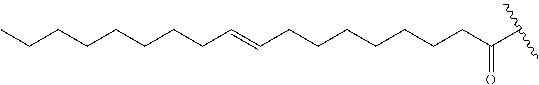 [SMILES: *C(=O)CCCCCCC/C=C/CCCCCCCC] | 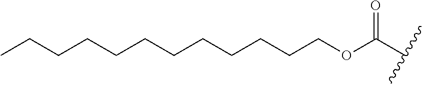 [SMILES: *C(=O)OCCCCCCCCCCCC] |
| 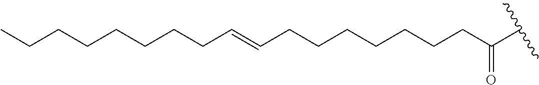 [SMILES: *C(=O)CCCCCCC/C=C/CCCCCCCC] | 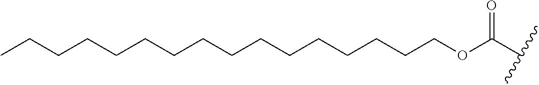 [SMILES: *C(=O)OCCCCCCCCCCCCCCCC] |
| 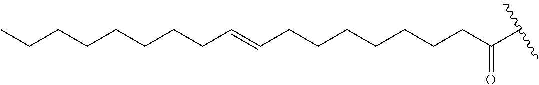 [SMILES: *C(=O)CCCCCCC/C=C/CCCCCCCC] | 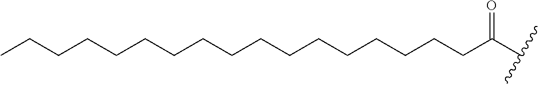 [SMILES: *C(=O)CCCCCCCCCCCCCCCCC] |
| 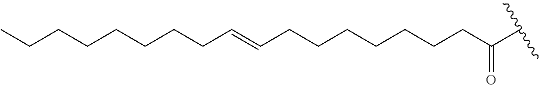 [SMILES: *C(=O)CCCCCCC/C=C/CCCCCCCC] | 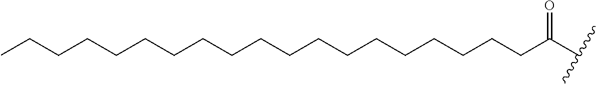 [SMILES: *C(=O)CCCCCCCCCCCCCCCCCCC] |
| 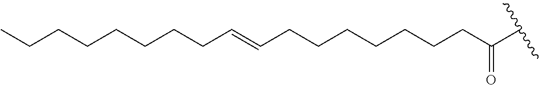 [SMILES: *C(=O)CCCCCCC/C=C/CCCCCCCC] | 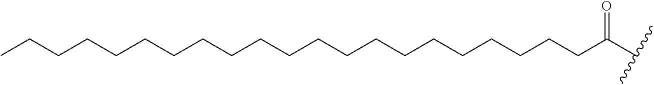 [SMILES: *C(=O)CCCCCCCCCCCCCCCCCCCCC] |
| 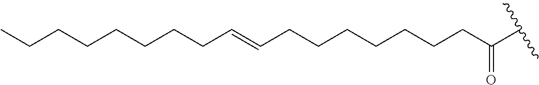 [SMILES: *C(=O)CCCCCCC/C=C/CCCCCCCC] | 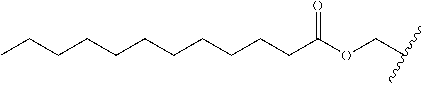 [SMILES: *COC(=O)CCCCCCCCCCC] |
| 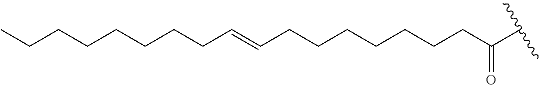 [SMILES: *C(=O)CCCCCCC/C=C/CCCCCCCC] | 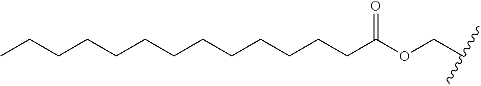 [SMILES: *COC(=O)CCCCCCCCCCCCC] |
| 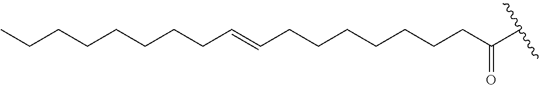 [SMILES: *C(=O)CCCCCCC/C=C/CCCCCCCC] | 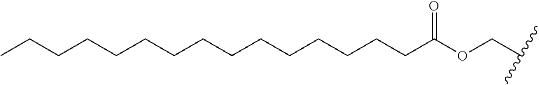 [SMILES: *COC(=O)CCCCCCCCCCCCCCC] |
| 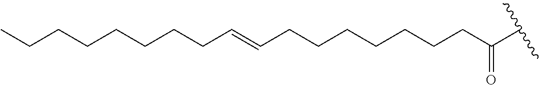 [SMILES: *C(=O)CCCCCCC/C=C/CCCCCCCC] | 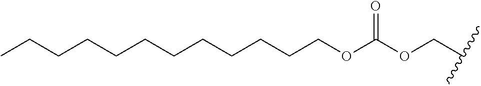 [SMILES: *COC(=O)OCCCCCCCCCCCC] |
| 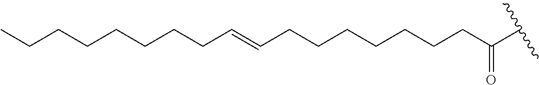 [SMILES: *C(=O)CCCCCCC/C=C/CCCCCCCC] | 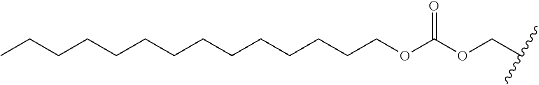 [SMILES: *COC(=O)OCCCCCCCCCCCCCC] |
| 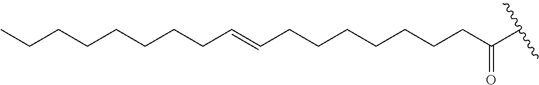 [SMILES: *C(=O)CCCCCCC/C=C/CCCCCCCC] | 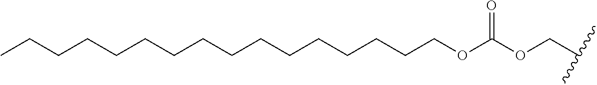 [SMILES: *COC(=O)OCCCCCCCCCCCCCCCC] |
| 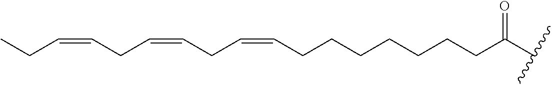 [SMILES: *C(=O)CCCCCCC/C=C\C/C=C\C/C=C\CC] | 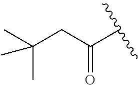 [SMILES: *C(=O)CC(C)(C)C] |
| 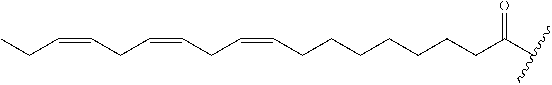 [SMILES: *C(=O)CCCCCCC/C=C\C/C=C\C/C=C\CC] | 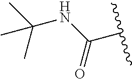 [SMILES: *C(=O)NC(C)(C)C] |
| 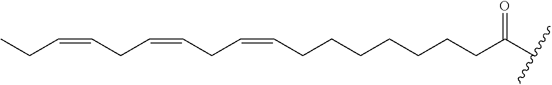 [SMILES: *C(=O)CCCCCCC/C=C\C/C=C\C/C=C\CC] | 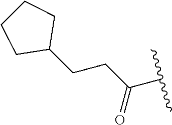 [SMILES: *C(=O)CCC1CCCC1] |
| 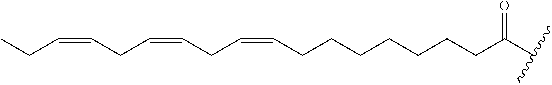 [SMILES: *C(=O)CCCCCCC/C=C\C/C=C\C/C=C\CC] | 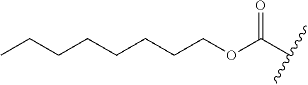 [SMILES: *C(=O)OCCCCCCCC] |
| 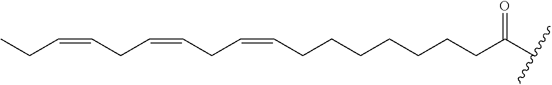 [SMILES: *C(=O)CCCCCCC/C=C\C/C=C\C/C=C\CC] | 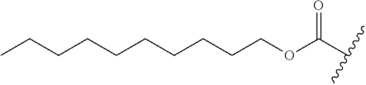 [SMILES: *C(=O)OCCCCCCCCCC] |
| 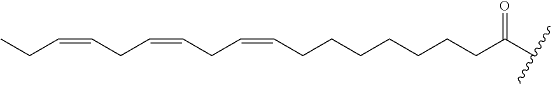 [SMILES: *C(=O)CCCCCCC/C=C\C/C=C\C/C=C\CC] | 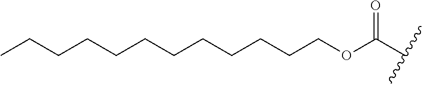 [SMILES: *C(=O)OCCCCCCCCCCCC] |
| 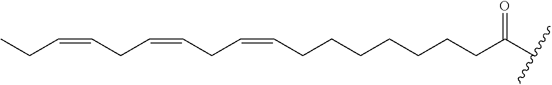 [SMILES: *C(=O)CCCCCCC/C=C\C/C=C\C/C=C\CC] | 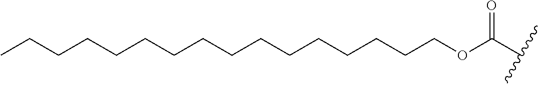 [SMILES: *C(=O)OCCCCCCCCCCCCCCCC] |
| 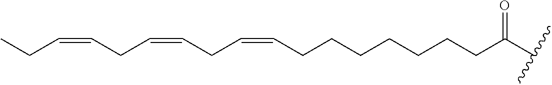 [SMILES: *C(=O)CCCCCCC/C=C\C/C=C\C/C=C\CC] | 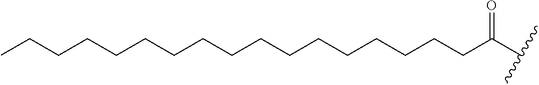 [SMILES: *C(=O)CCCCCCCCCCCCCCCCC] |
| 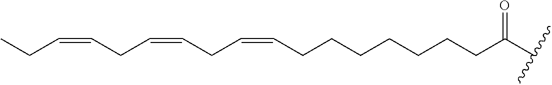 [SMILES: *C(=O)CCCCCCC/C=C\C/C=C\C/C=C\CC] | 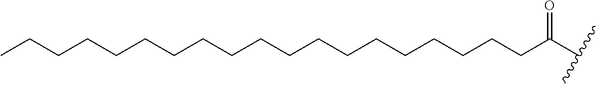 [SMILES: *C(=O)CCCCCCCCCCCCCCCCCCC] |
| 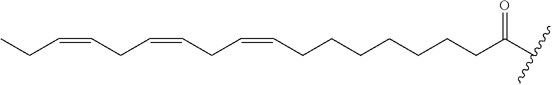 [SMILES: *C(=O)CCCCCCC/C=C\C/C=C\C/C=C\CC] | 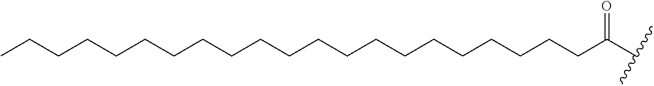 [SMILES: *C(=O)CCCCCCCCCCCCCCCCCCCCC] |
| 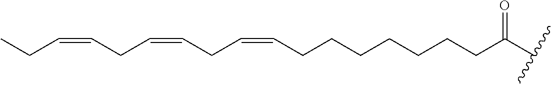 [SMILES: *C(=O)CCCCCCC/C=C\C/C=C\C/C=C\CC] | 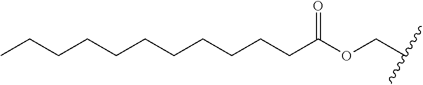 [SMILES: *COC(=O)CCCCCCCCCCC] |
| 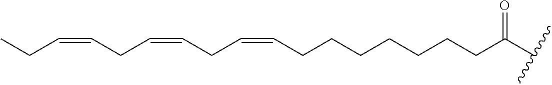 [SMILES: *C(=O)CCCCCCC/C=C\C/C=C\C/C=C\CC] | 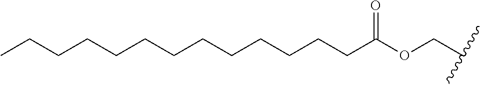 [SMILES: *COC(=O)CCCCCCCCCCCCC] |
| 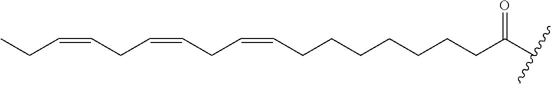 [SMILES: *C(=O)CCCCCCC/C=C\C/C=C\C/C=C\CC] | 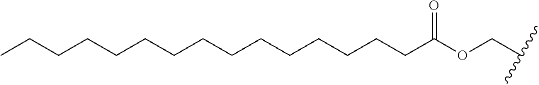 [SMILES: *COC(=O)CCCCCCCCCCCCCCC] |
| 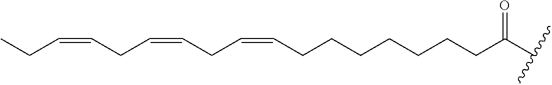 [SMILES: *C(=O)CCCCCCC/C=C\C/C=C\C/C=C\CC] | 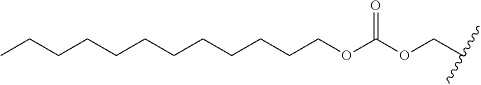 [SMILES: *COC(=O)OCCCCCCCCCCCC] |
| 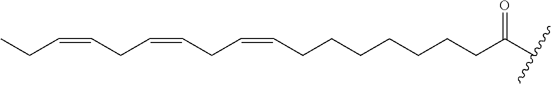 [SMILES: *C(=O)CCCCCCC/C=C\C/C=C\C/C=C\CC] | 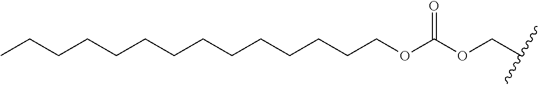 [SMILES: *COC(=O)OCCCCCCCCCCCCCC] |
| 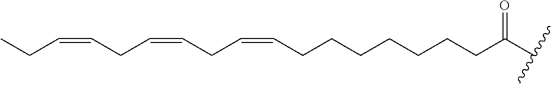 [SMILES: *C(=O)CCCCCCC/C=C\C/C=C\C/C=C\CC] | 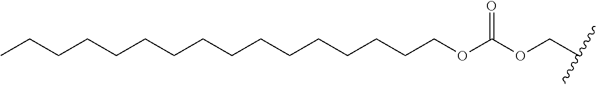 [SMILES: *COC(=O)OCCCCCCCCCCCCCCCC] |
| tBu | 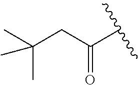 [SMILES: *C(=O)CC(C)(C)C] |
| tBu | 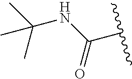 [SMILES: *C(=O)NC(C)(C)C] |
| tBu | 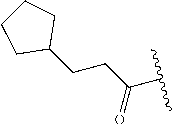 [SMILES: *C(=O)CCC1CCCC1] |
| tBu | 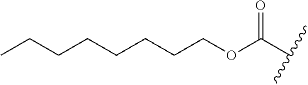 [SMILES: *C(=O)OCCCCCCCC] |
| tBu | 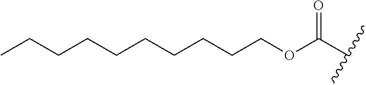 [SMILES: *C(=O)OCCCCCCCCCC] |
| tBu | 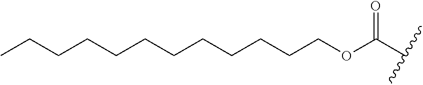 [SMILES: *C(=O)OCCCCCCCCCCCC] |
| tBu | 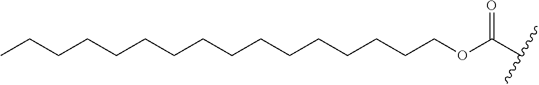 [SMILES: *C(=O)OCCCCCCCCCCCCCCCC] |
| tBu | 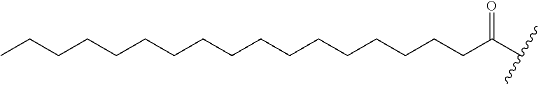 [SMILES: *C(=O)CCCCCCCCCCCCCCCCC] |
| tBu | 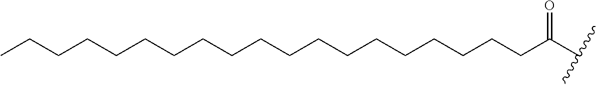 [SMILES: *C(=O)CCCCCCCCCCCCCCCCCCC] |
| tBu | 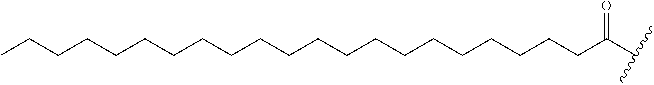 [SMILES: *C(=O)CCCCCCCCCCCCCCCCCCCCC] |
| tBu | 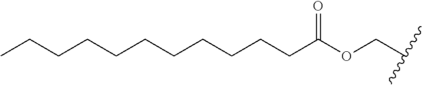 [SMILES: *COC(=O)CCCCCCCCCCC] |
| tBu | 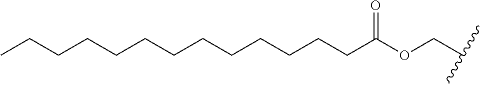 [SMILES: *COC(=O)CCCCCCCCCCCCC] |
| tBu | 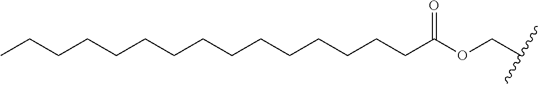 [SMILES: *COC(=O)CCCCCCCCCCCCCCC] |
| tBu | 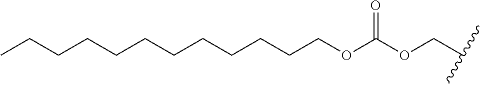 [SMILES: *COC(=O)OCCCCCCCCCCCC] |
| tBu | 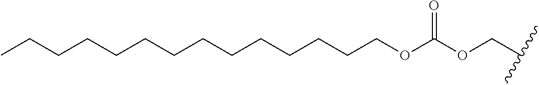 [SMILES: *COC(=O)OCCCCCCCCCCCCCC] |
| tBu | 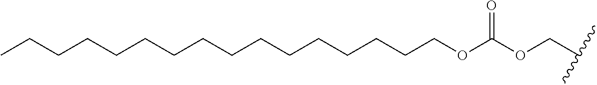 [SMILES: *COC(=O)OCCCCCCCCCCCCCCCC] |
| phenyl | 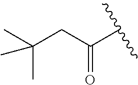 [SMILES: *C(=O)CC(C)(C)C] |
| phenyl | 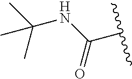 [SMILES: *C(=O)NC(C)(C)C] |
| phenyl | 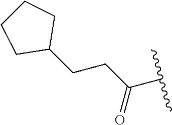 [SMILES: *C(=O)CCC1CCCC1] |
| phenyl | 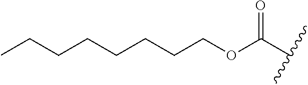 [SMILES: *C(=O)OCCCCCCCC] |
| phenyl | 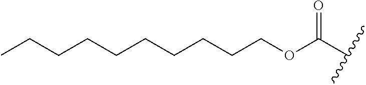 [SMILES: *C(=O)OCCCCCCCCCC] |
| phenyl | 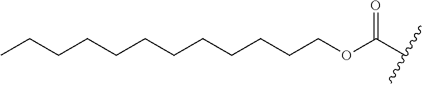 [SMILES: *C(=O)OCCCCCCCCCCCC] |
| phenyl | 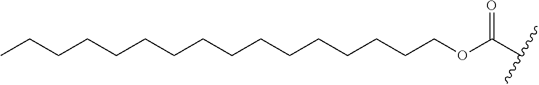 [SMILES: *C(=O)OCCCCCCCCCCCCCCCC] |
| phenyl | 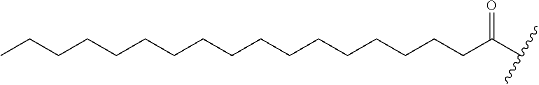 [SMILES: *C(=O)CCCCCCCCCCCCCCCCC] |
| phenyl | 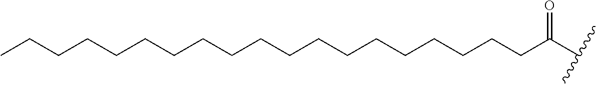 [SMILES: *C(=O)CCCCCCCCCCCCCCCCCCC] |
| phenyl | 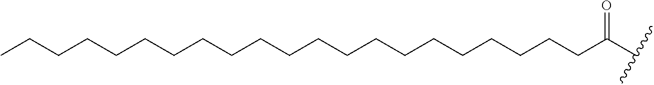 [SMILES: *C(=O)CCCCCCCCCCCCCCCCCCCCC] |
| phenyl | 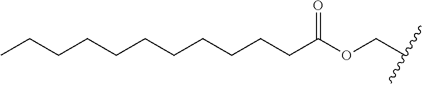 [SMILES: *COC(=O)CCCCCCCCCCC] |
| phenyl | 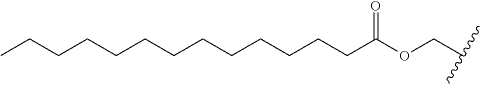 [SMILES: *COC(=O)CCCCCCCCCCCCC] |
| phenyl | 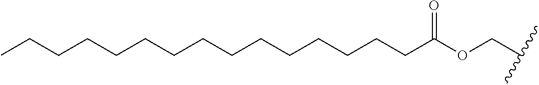 [SMILES: *COC(=O)CCCCCCCCCCCCCCC] |
| phenyl | 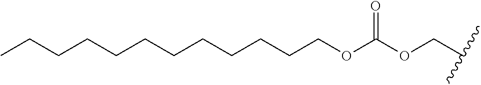 [SMILES: *COC(=O)OCCCCCCCCCCCC] |
| phenyl | 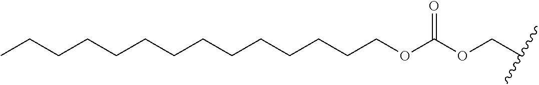 [SMILES: *COC(=O)OCCCCCCCCCCCCCC] |
| phenyl | 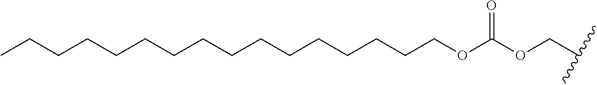 [SMILES: *COC(=O)OCCCCCCCCCCCCCCCC] |
| 4-chlorophenyl | 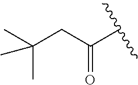 [SMILES: *C(=O)CC(C)(C)C] |
| 4-chlorophenyl | 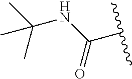 [SMILES: *C(=O)NC(C)(C)C] |
| 4-chlorophenyl | 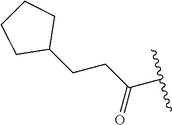 [SMILES: *C(=O)CCC1CCCC1] |
| 4-chlorophenyl | 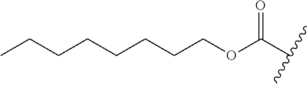 [SMILES: *C(=O)OCCCCCCCC] |
| 4-chlorophenyl | 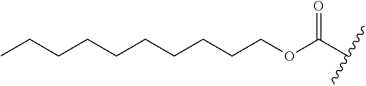 [SMILES: *C(=O)OCCCCCCCCCC] |
| 4-chlorophenyl | 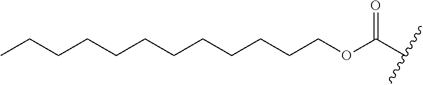 [SMILES: *C(=O)OCCCCCCCCCCCC] |
| 4-chlorophenyl | 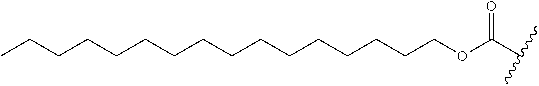 [SMILES: *C(=O)OCCCCCCCCCCCCCCCC] |
| 4-chlorophenyl | 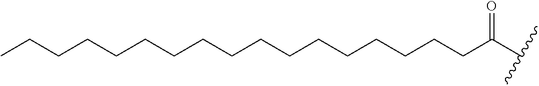 [SMILES: *C(=O)CCCCCCCCCCCCCCCCC] |
| 4-chlorophenyl | 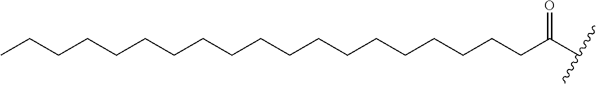 [SMILES: *C(=O)CCCCCCCCCCCCCCCCCCC] |
| 4-chlorophenyl | 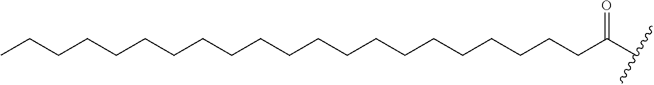 [SMILES: *C(=O)CCCCCCCCCCCCCCCCCCCCC] |
| 4-chlorophenyl | 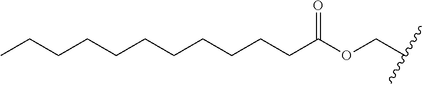 [SMILES: *COC(=O)CCCCCCCCCCC] |
| 4-chlorophenyl | 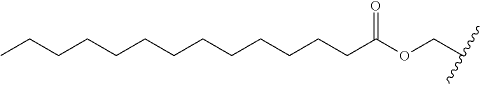 [SMILES: *COC(=O)CCCCCCCCCCCCC] |
| 4-chlorophenyl | 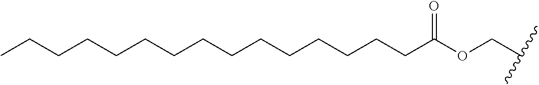 [SMILES: *COC(=O)CCCCCCCCCCCCCCC] |
| 4-chlorophenyl | 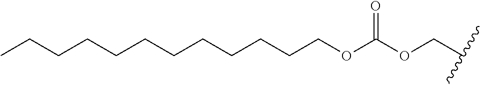 [SMILES: *COC(=O)OCCCCCCCCCCCC] |
| 4-chlorophenyl | 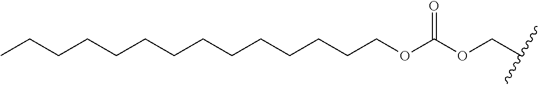 [SMILES: *COC(=O)OCCCCCCCCCCCCCC] |
| 4-chlorophenyl | 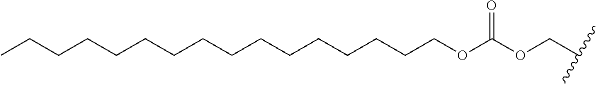 [SMILES: *COC(=O)OCCCCCCCCCCCCCCCC] |
| 3,4-dichlorophenyl | 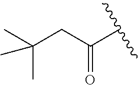 [SMILES: *C(=O)CC(C)(C)C] |
| 3,4-dichlorophenyl | 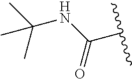 [SMILES: *C(=O)NC(C)(C)C] |
| 3,4-dichlorophenyl | 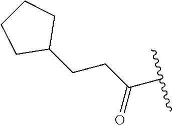 [SMILES: *C(=O)CCC1CCCC1] |
| 3,4-dichlorophenyl | 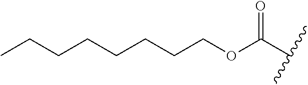 [SMILES: *C(=O)OCCCCCCCC] |
| 3,4-dichlorophenyl | 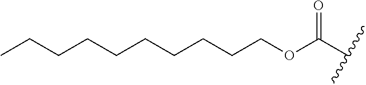 [SMILES: *C(=O)OCCCCCCCCCC] |
| 3,4-dichlorophenyl | 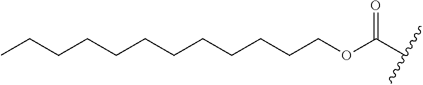 [SMILES: *C(=O)OCCCCCCCCCCCC] |
| 3,4-dichlorophenyl | 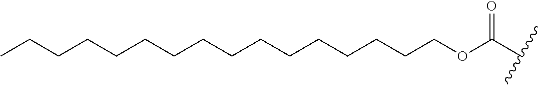 [SMILES: *C(=O)OCCCCCCCCCCCCCCCC] |
| 3,4-dichlorophenyl | 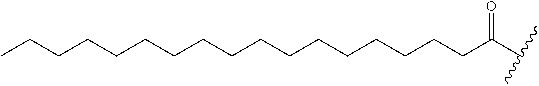 [SMILES: *C(=O)CCCCCCCCCCCCCCCCC] |
| 3,4-dichlorophenyl | 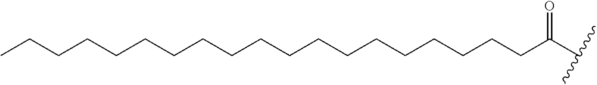 [SMILES: *C(=O)CCCCCCCCCCCCCCCCCCC] |
| 3,4-dichlorophenyl | 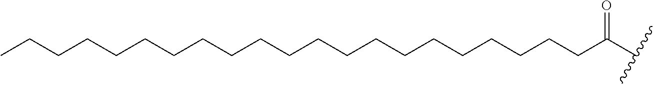 [SMILES: *C(=O)CCCCCCCCCCCCCCCCCCCCC] |
| 3,4-dichlorophenyl | 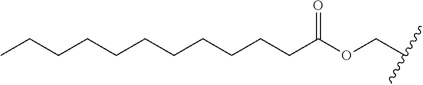 [SMILES: *COC(=O)CCCCCCCCCCC] |
| 3,4-dichlorophenyl | 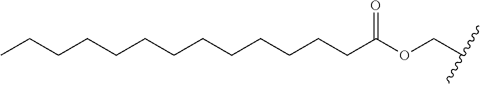 [SMILES: *COC(=O)CCCCCCCCCCCCC] |
| 3,4-dichlorophenyl | 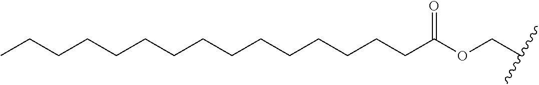 [SMILES: *COC(=O)CCCCCCCCCCCCCCC] |
| 3,4-dichlorophenyl | 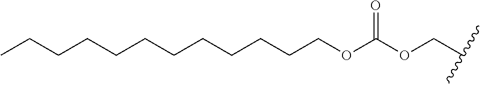 [SMILES: *COC(=O)OCCCCCCCCCCCC] |
| 3,4-dichlorophenyl | 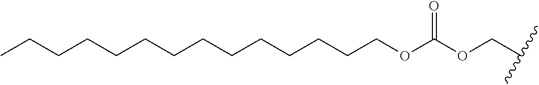 [SMILES: *COC(=O)OCCCCCCCCCCCCCC] |
| 3,4-dichlorophenyl | 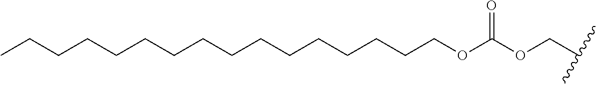 [SMILES: *COC(=O)OCCCCCCCCCCCCCCCC] |
| 4-methylphenyl | 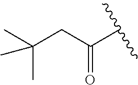 [SMILES: *C(=O)CC(C)(C)C] |
| 4-methylphenyl | 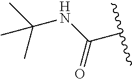 [SMILES: *C(=O)NC(C)(C)C] |
| 4-methylphenyl | 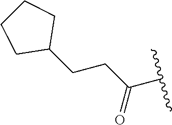 [SMILES: *C(=O)CCC1CCCC1] |
| 4-methylphenyl | 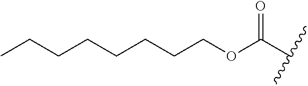 [SMILES: *C(=O)OCCCCCCCC] |
| 4-methylphenyl | 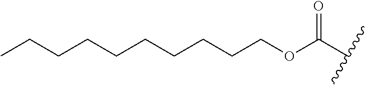 [SMILES: *C(=O)OCCCCCCCCCC] |
| 4-methylphenyl | 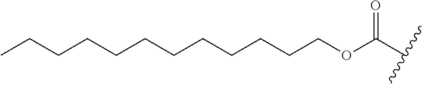 [SMILES: *C(=O)OCCCCCCCCCCCC] |
| 4-methylphenyl | 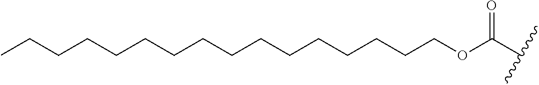 [SMILES: *C(=O)OCCCCCCCCCCCCCCCC] |
| 4-methylphenyl | 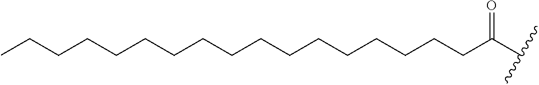 [SMILES: *C(=O)CCCCCCCCCCCCCCCCC] |
| 4-methylphenyl | 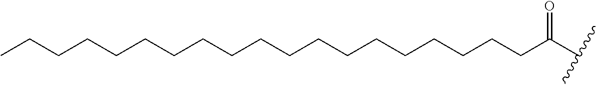 [SMILES: *C(=O)CCCCCCCCCCCCCCCCCCC] |
| 4-methylphenyl | 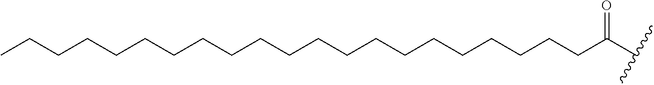 [SMILES: *C(=O)CCCCCCCCCCCCCCCCCCCCC] |
| 4-methylphenyl | 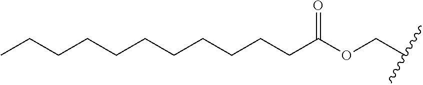 [SMILES: *COC(=O)CCCCCCCCCCC] |
| 4-methylphenyl | 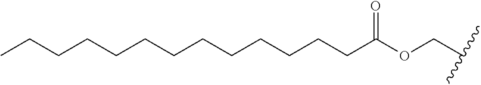 [SMILES: *COC(=O)CCCCCCCCCCCCC] |
| 4-methylphenyl | 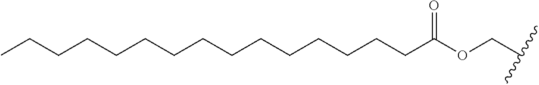 [SMILES: *COC(=O)CCCCCCCCCCCCCCC] |
| 4-methylphenyl | 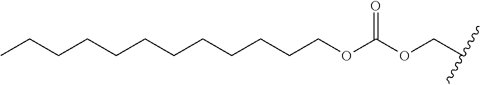 [SMILES: *COC(=O)OCCCCCCCCCCCC] |
| 4-methylphenyl | 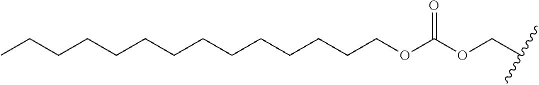 [SMILES: *COC(=O)OCCCCCCCCCCCCCC] |
| 4-methylphenyl | 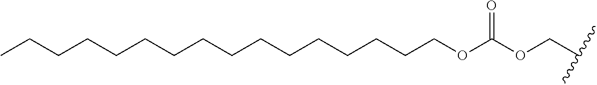 [SMILES: *COC(=O)OCCCCCCCCCCCCCCCC] |
| 4-methoxyphenyl | 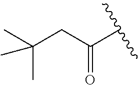 [SMILES: *C(=O)CC(C)(C)C] |
| 4-methoxyphenyl | 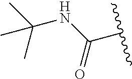 [SMILES: *C(=O)NC(C)(C)C] |
| 4-methoxyphenyl | 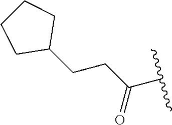 [SMILES: *C(=O)CCC1CCCC1] |
| 4-methoxyphenyl | 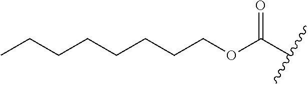 [SMILES: *C(=O)OCCCCCCCC] |
| 4-methoxyphenyl | 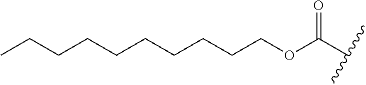 [SMILES: *C(=O)OCCCCCCCCCC] |
| 4-methoxyphenyl | 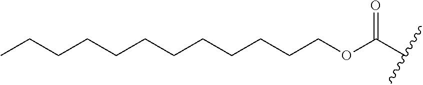 [SMILES: *C(=O)OCCCCCCCCCCCC] |
| 4-methoxyphenyl | 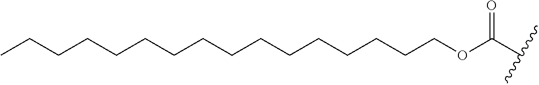 [SMILES: *C(=O)OCCCCCCCCCCCCCCCC] |
| 4-methoxyphenyl | 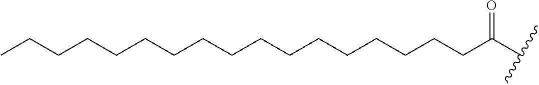 [SMILES: *C(=O)CCCCCCCCCCCCCCCCC] |
| 4-methoxyphenyl | 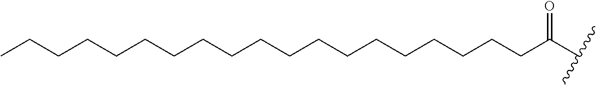 [SMILES: *C(=O)CCCCCCCCCCCCCCCCCCC] |
| 4-methoxyphenyl | 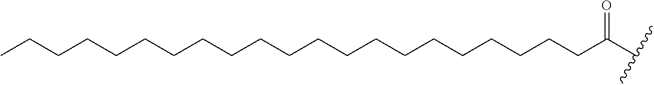 [SMILES: *C(=O)CCCCCCCCCCCCCCCCCCCCC] |
| 4-methoxyphenyl | 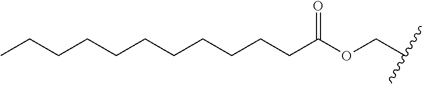 [SMILES: *COC(=O)CCCCCCCCCCC] |
| 4-methoxyphenyl | 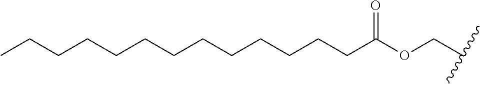 [SMILES: *COC(=O)CCCCCCCCCCCCC] |
| 4-methoxyphenyl | 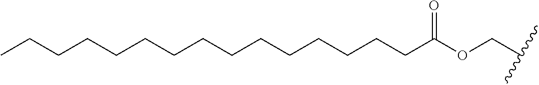 [SMILES: *COC(=O)CCCCCCCCCCCCCCC] |
| 4-methoxyphenyl | 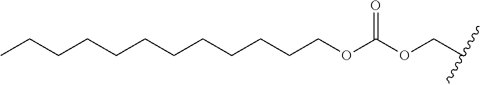 [SMILES: *COC(=O)OCCCCCCCCCCCC] |
| 4-methoxyphenyl | 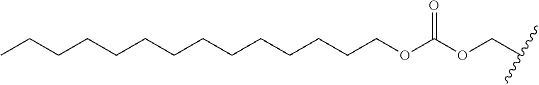 [SMILES: *COC(=O)OCCCCCCCCCCCCCC] |
| 4-methoxyphenyl | 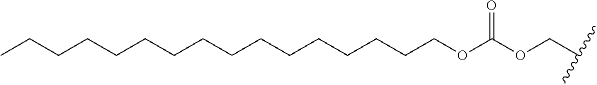 [SMILES: *COC(=O)OCCCCCCCCCCCCCCCC] |

In a 52^{nd} aspect, provided herein is a compound, or pharmaceutically acceptable salt thereof, having a structure provided in Formula (I-17),

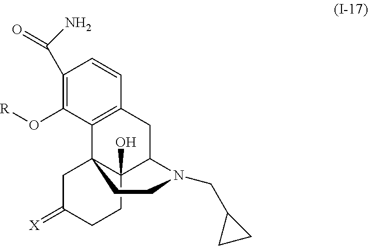

wherein X is ═O, R is R⁴C(O)—O—CH(R³)— and R³ and R⁴ are any of the pair of R³/R⁴ groups recited in Table 7. Alternatively, X is ═CH₂, R is R⁴C(O)—O—CH(R³)— and R³ and R⁴ are any of the pair of R³/R⁴ groups recited in Table 7.

In a 53^{rd} aspect, provided herein is a compound, or pharmaceutically acceptable salt thereof, having a structure provided in Formula (II-17),

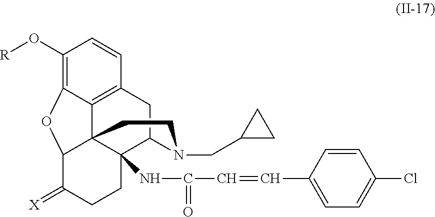

wherein X is ═O, R is R⁴C(O)—O—CH(R³)— and R³ and R⁴ are any of the pair of R³/R⁴ groups recited in Table 7. Alternatively, X is ═CH₂, R is R⁴C(O)—O—CH(R³)— and R³ and R⁴ are any of the pair of R³/R⁴ groups recited in Table 7.

In a 54^{th} aspect, provided herein is a compound, or pharmaceutically acceptable salt thereof, having a structure provided in Formula (III-17),

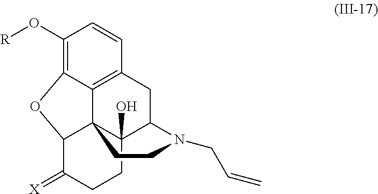

wherein X is ═O, R is R⁴C(O)—O—CH(R³)— and R³ and R⁴ are any of the pair of R³/R⁴ groups recited in Table 7. Alternatively, X is ═CH₂, R is R⁴C(O)—O—CH(R³)— and R³ and R⁴ are any of the pair of R³/R⁴ groups recited in Table 7.

TABLE 7

| R³ | R⁴ |
|---|---|
| H | Me |
| H | —C₇H₁₅ |
| H | —C₉H₁₉ |
| H | —C₁₁H₂₃ |
| H | —C₁₃H₂₇ |
| H | —C₁₅H₃₁ |
| H | —C₁₇H₃₅ |
| H | 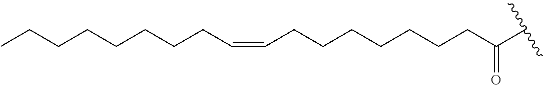 [SMILES: *C(=O)CCCCCCC/C=C\CCCCCCCC] |
| H | 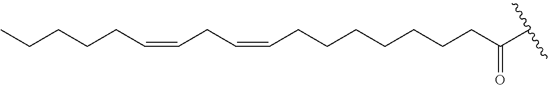 [SMILES: *C(=O)CCCCCCC/C=C\C/C=C\CCCCC] |
| H | 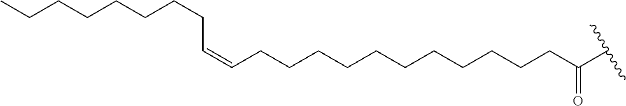 [SMILES: *C(=O)CCCCCCCCCCC/C=C\CCCCCCCC] |
| H | 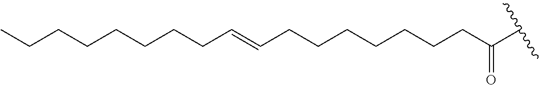 [SMILES: *C(=O)CCCCCCC/C=C/CCCCCCCC] |
| H | 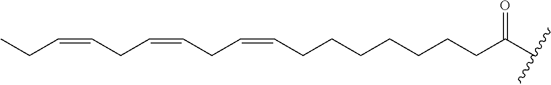 [SMILES: *C(=O)CCCCCCC/C=C\C/C=C\C/C=C\CC] |
| H | tBu |
| H | phenyl |
| H | 4-chlorophenyl |
| H | 3,4-dichlorophenyl |
| H | 4-methylphenyl |
| H | 4-methoxyphenyl |
| Me | Me |
| Me | —C₇H₁₅ |
| Me | —C₉H₁₉ |
| Me | —C₁₁H₂₃ |
| Me | —C₁₃H₂₇ |
| Me | —C₁₅H₃₁ |
| Me | —C₁₇H₃₅ |
| Me | 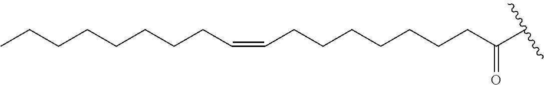 [SMILES: *C(=O)CCCCCCC/C=C\CCCCCCCC] |
| Me | 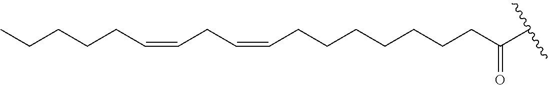 [SMILES: *C(=O)CCCCCCC/C=C\C/C=C\CCCCC] |
| Me | 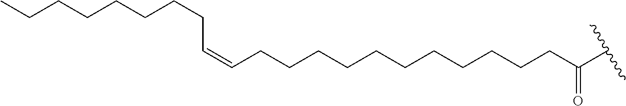 [SMILES: *C(=O)CCCCCCCCCCC/C=C\CCCCCCCC] |
| Me | 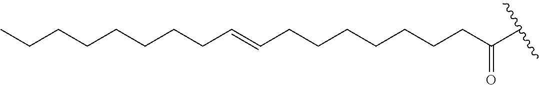 [SMILES: *C(=O)CCCCCCC/C=C/CCCCCCCC] |
| Me | 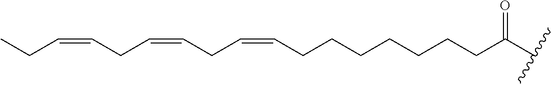 [SMILES: *C(=O)CCCCCCC/C=C\C/C=C\C/C=C\CC] |
| Me | tBu |
| Me | phenyl |
| Me | 4-chlorophenyl |
| Me | 3,4-dichlorophenyl |
| Me | 4-methylphenyl |
| Me | 4-methoxyphenyl |
| tBu | Me |
| tBu | —C₇H₁₅ |
| tBu | —C₉H₁₉ |
| tBu | —C₁₁H₂₃ |
| tBu | —C₁₃H₂₇ |
| tBu | —C₁₅H₃₁ |
| tBu | —C₁₇H₃₅ |
| tBu | 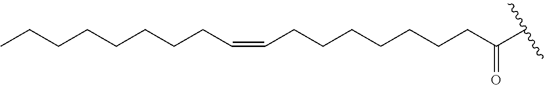 [SMILES: *C(=O)CCCCCCC/C=C\CCCCCCCC] |
| tBu | 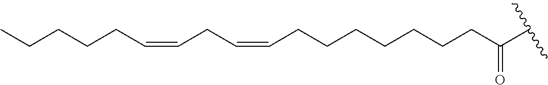 [SMILES: *C(=O)CCCCCCC/C=C\C/C=C\CCCCC] |
| tBu | 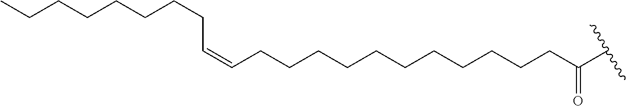 [SMILES: *C(=O)CCCCCCCCCCC/C=C\CCCCCCCC] |
| tBu | 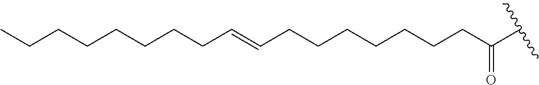 [SMILES: *C(=O)CCCCCCC/C=C/CCCCCCCC] |
| tBu | 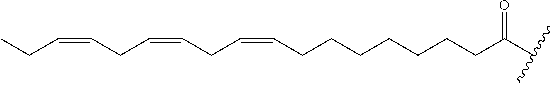 [SMILES: *C(=O)CCCCCCC/C=C\C/C=C\C/C=C\CC] |
| tBu | phenyl |
| tBu | tBu |
| tBu | 4-chlorophenyl |
| tBu | 3,4-dichlorophenyl |
| tBu | 4-methylphenyl |
| tBu | 4-methoxyphenyl |
| —C₇H₁₅ | Me |
| —C₇H₁₅ | —C₇H₁₅ |
| —C₇H₁₅ | —C₉H₁₉ |
| —C₇H₁₅ | —C₁₁H₂₃ |
| —C₇H₁₅ | —C₁₃H₂₇ |
| —C₇H₁₅ | —C₁₅H₃₁ |
| —C₇H₁₅ | —C₁₇H₃₅ |
| —C₇H₁₅ | 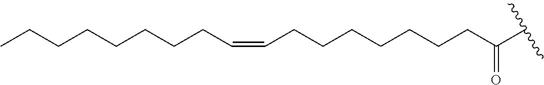 [SMILES: *C(=O)CCCCCCC/C=C\CCCCCCCC] |
| —C₇H₁₅ | 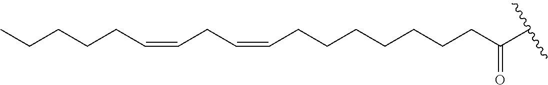 [SMILES: *C(=O)CCCCCCC/C=C\C/C=C\CCCCC] |
| —C₇H₁₅ | 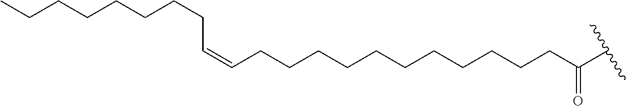 [SMILES: *C(=O)CCCCCCCCCCC/C=C\CCCCCCCC] |
| —C₇H₁₅ | 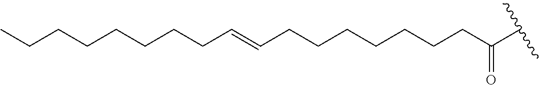 [SMILES: *C(=O)CCCCCCC/C=C/CCCCCCCC] |
| —C7H₁₅ | 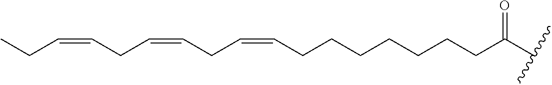 [SMILES: *C(=O)CCCCCCC/C=C\C/C=C\C/C=C\CC] |
| —C₇H₁₅ | tBu |
| —C₇H₁₅ | phenyl |
| —C₇H₁₅ | 4-chlorophenyl |
| —C₇H₁₅ | 3,4-dichlorophenyl |
| —C₇H₁₅ | 4-methylphenyl |
| —C₇H₁₅ | 4-methoxyphenyl |
| —C₉H₁₉ | Me |
| —C₉H₁₉ | —C₇H₁₅ |
| —C₉H₁₉ | —C₉H₁₉ |
| —C₉H₁₉ | —C₁₁H₂₃ |
| —C₉H₁₉ | —C₁₃H₂₇ |
| —C₉H₁₉ | —C₁₅H₃₁ |
| —C₉H₁₉ | —C₁₇H₃₅ |
| —C₉H₁₉ | 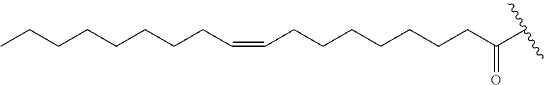 [SMILES: *C(=O)CCCCCCC/C=C\CCCCCCCC] |
| —C₉H₁₉ | 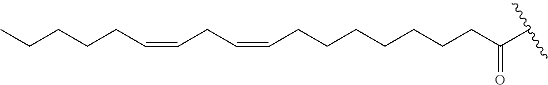 [SMILES: *C(=O)CCCCCCC/C=C\C/C=C\CCCCC] |
| —C₉H₁₉ | 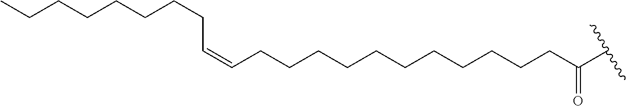 [SMILES: *C(=O)CCCCCCCCCCC/C=C\CCCCCCCC] |
| —C₉H₁₉ | 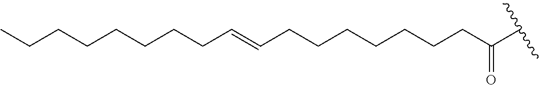 [SMILES: *C(=O)CCCCCCC/C=C/CCCCCCCC] |
| —C₉H₁₉ | 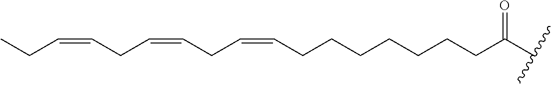 [SMILES: *C(=O)CCCCCCC/C=C\C/C=C\C/C=C\CC] |
| —C₉H₁₉ | tBu |
| —C₉H₁₉ | phenyl |
| —C₉H₁₉ | 4-chlorophenyl |
| —C₉H₁₉ | 3,4-dichlorophenyl |
| —C₉H₁₉ | 4-methylphenyl |
| —C₉H₁₉ | 4-methoxyphenyl |
| —C₁₁H₂₃ | Me |
| —C₁₁H₂₃ | —C₇H₁₅ |
| —C₁₁H₂₃ | —C₉H₁₉ |
| —C₁₁H₂₃ | —C₁₁H₂₃ |
| —C₁₁H₂₃ | —C₁₃H₂₇ |
| —C₁₁H₂₃ | —C₁₅H₃₁ |
| —C₁₁H₂₃ | —C₁₇H₃₅ |
| —C₁₁H₂₃ | 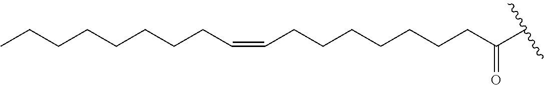 [SMILES: *C(=O)CCCCCCC/C=C\CCCCCCCC] |
| —C₁₁H₂₃ | 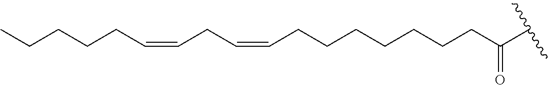 [SMILES: *C(=O)CCCCCCC/C=C\C/C=C\CCCCC] |
| —C₁₁H₂₃ | 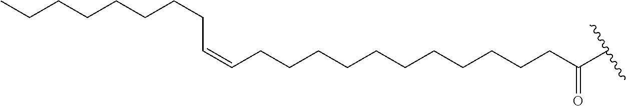 [SMILES: *C(=O)CCCCCCCCCCC/C=C\CCCCCCCC] |
| —C₁₁H₂₃ | 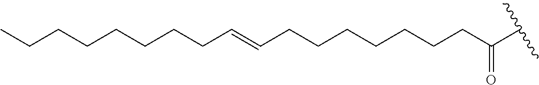 [SMILES: *C(=O)CCCCCCC/C=C/CCCCCCCC] |
| —C₁₁H₂₃ | 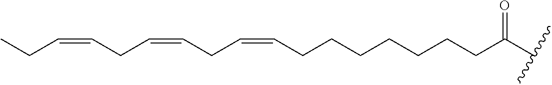 [SMILES: *C(=O)CCCCCCC/C=C\C/C=C\C/C=C\CC] |
| —C₁₁H₂₃ | tBu |
| —C₁₁H₂₃ | phenyl |
| —C₁₁H₂₃ | 4-chlorophenyl |
| —C₁₁H₂₃ | 3,4-dichlorophenyl |
| —C₁₁H₂₃ | 4-methylphenyl |
| —C₁₁H₂₃ | 4-methoxyphenyl |
| —C₁₃H₂₇ | Me |
| —C₁₃H₂₇ | —C₇H₁₅ |
| —C₁₃H₂₇ | —C₉H₁₉ |
| —C₁₃H₂₇ | —C₁₁H₂₃ |
| —C₁₃H₂₇ | —C₁₃H₂₇ |
| —C₁₃H₂₇ | —C₁₅H₃₁ |
| —C₁₃H₂₇ | —C₁₇H₃₅ |
| —C₁₃H₂₇ | 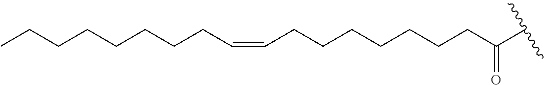 [SMILES: *C(=O)CCCCCCC/C=C\CCCCCCCC] |
| —C₁₃H₂₇ | 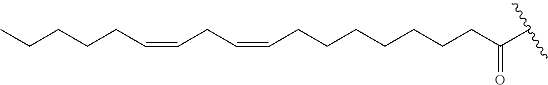 [SMILES: *C(=O)CCCCCCC/C=C\C/C=C\CCCCC] |
| —C₁₃H₂₇ | 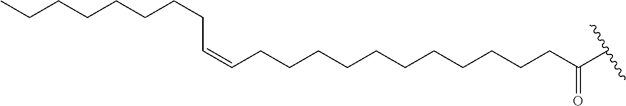 [SMILES: *C(=O)CCCCCCCCCCC/C=C\CCCCCCCC] |
| —C₁₃H₂₇ | 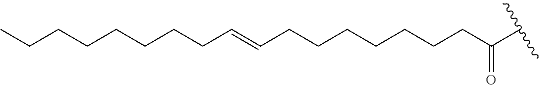 [SMILES: *C(=O)CCCCCCC/C=C/CCCCCCCC] |
| —C₁₃H₂₇ | 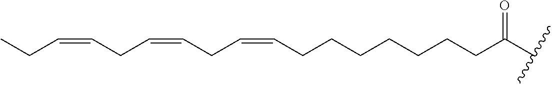 [SMILES: *C(=O)CCCCCCC/C=C\C/C=C\C/C=C\CC] |
| —C₁₃H₂₇ | tBu |
| —C₁₃H₂₇ | phenyl |
| —C₁₃H₂₇ | 4-chlorophenyl |
| —C₁₃H₂₇ | 3,4-dichlorophenyl |
| —C₁₃H₂₇ | 4-methylphenyl |
| —C₁₃H₂₇ | 4-methoxyphenyl |
| —C₁₅H₃₁ | Me |
| —C₁₅H₃₁ | —C₇H₁₅ |
| —C₁₅H₃₁ | —C₉H₁₉ |
| —C₁₅H₃₁ | —C₁₁H₂₃ |
| —C₁₅H₃₁ | —C₁₃H₂₇ |
| —C₁₅H₃₁ | —C₁₅H₃₁ |
| —C₁₅H₃₁ | —C₁₇H₃₅ |
| —C₁₅H₃₁ | 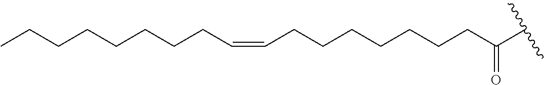 [SMILES: *C(=O)CCCCCCC/C=C\CCCCCCCC] |
| —C₁₅H₃₁ | 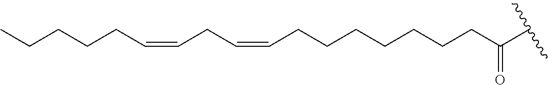 [SMILES: *C(=O)CCCCCCC/C=C\C/C=C\CCCCC] |
| —C₁₅H₃₁ | 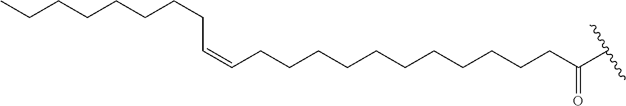 [SMILES: *C(=O)CCCCCCCCCCC/C=C\CCCCCCCC] |
| —C₁₅H₃₁ | 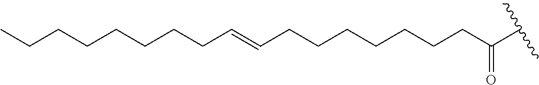 [SMILES: *C(=O)CCCCCCC/C=C/CCCCCCCC] |
| —C₁₅H₃₁ | 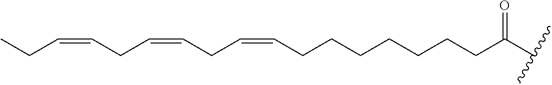 [SMILES: *C(=O)CCCCCCC/C=C\C/C=C\C/C=C\CC] |
| —C₁₅H₃₁ | tBu |
| —C₁₅H₃₁ | phenyl |
| —C₁₅H₃₁ | 4-chlorophenyl |
| —C₁₅H₃₁ | 3,4-dichlorophenyl |
| —C₁₅H₃₁ | 4-methylphenyl |
| —C₁₅H₃₁ | 4-methoxyphenyl |
| —C₁₇H₃₅ | Me |
| —C₁₇H₃₅ | —C₇H₁₅ |
| —C₁₇H₃₅ | —C₉H₁₉ |
| —C₁₇H₃₅ | —C₁₁H₂₃ |
| —C₁₇H₃₅ | —C₁₃H₂₇ |
| —C₁₇H₃₅ | —C₁₅H₃₁ |
| —C₁₇H₃₅ | —C₁₇H₃₅ |
| —C₁₇H₃₅ | 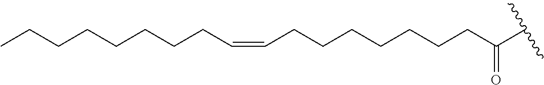 [SMILES: *C(=O)CCCCCCC/C=C\CCCCCCCC] |
| —C₁₇H₃₅ | 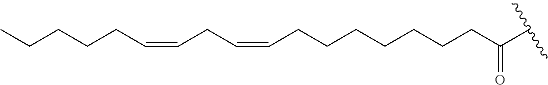 [SMILES: *C(=O)CCCCCCC/C=C\C/C=C\CCCCC] |
| —C₁₇H₃₅ | 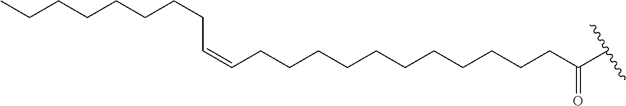 [SMILES: *C(=O)CCCCCCCCCCC/C=C\CCCCCCCC] |
| —C₁₇H₃₅ | 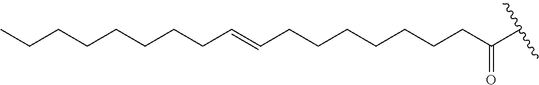 [SMILES: *C(=O)CCCCCCC/C=C/CCCCCCCC] |
| —C₁₇H₃₅ | 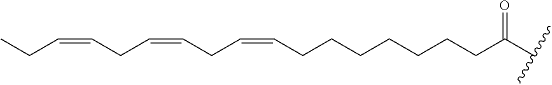 [SMILES: *C(=O)CCCCCCC/C=C\C/C=C\C/C=C\CC] |
| —C₁₇H₃₅ | tBu |
| —C₁₇H₃₅ | phenyl |
| —C₁₇H₃₅ | 4-chlorophenyl |
| —C₁₇H₃₅ | 3,4-dichlorophenyl |
| —C₁₇H₃₅ | 4-methylphenyl |
| —C₁₇H₃₅ | 4-methoxyphenyl |
| Phenyl | Me |
| Phenyl | —C₇H₁₅ |
| Phenyl | —C₉H₁₉ |
| Phenyl | —C₁₁H₂₃ |
| Phenyl | —C₁₃H₂₇ |
| Phenyl | —C₁₅H₃₁ |
| Phenyl | —C₁₇H₃₅ |
| Phenyl | 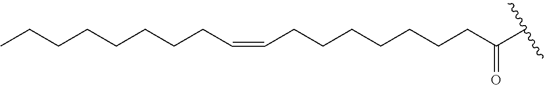 [SMILES: *C(=O)CCCCCCC/C=C\CCCCCCCC] |
| Phenyl | 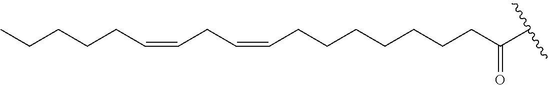 [SMILES: *C(=O)CCCCCCC/C=C\C/C=C\CCCCC] |
| Phenyl | 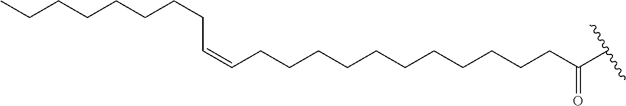 [SMILES: *C(=O)CCCCCCCCCCC/C=C\CCCCCCCC] |
| Phenyl | 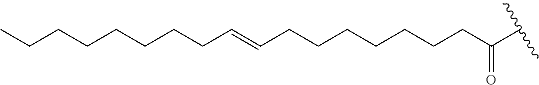 [SMILES: *C(=O)CCCCCCC/C=C/CCCCCCCC] |
| Phenyl | 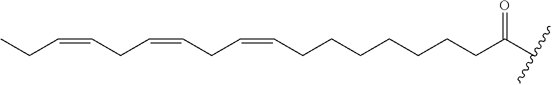 [SMILES: *C(=O)CCCCCCC/C=C\C/C=C\C/C=C\CC] |
| Phenyl | tBu |
| Phenyl | phenyl |
| Phenyl | 4-chlorophenyl |
| Phenyl | 3,4-dichlorophenyl |
| Phenyl | 4-methylphenyl |
| Phenyl | 4-methoxyphenyl |
| 4-chlorophenyl | Me |
| 4-chlorophenyl | —C₇H₁₅ |
| 4-chlorophenyl | —C₉H₁₉ |
| 4-chlorophenyl | —C₁₁H₂₃ |
| 4-chlorophenyl | —C₁₃H₂₇ |
| 4-chlorophenyl | —C₁₅H₃₁ |
| 4-chlorophenyl | —C₁₇H₃₅ |
| 4-chlorophenyl | 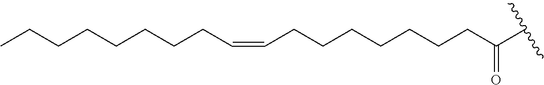 [SMILES: *C(=O)CCCCCCC/C=C\CCCCCCCC] |
| 4-chlorophenyl | 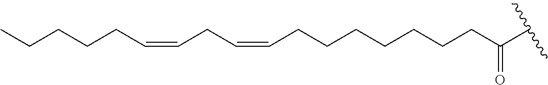 [SMILES: *C(=O)CCCCCCC/C=C\C/C=C\CCCCC] |
| 4-chlorophenyl | 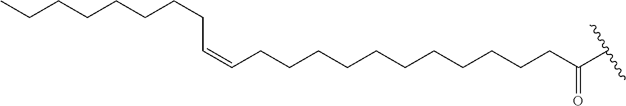 [SMILES: *C(=O)CCCCCCCCCCC/C=C\CCCCCCCC] |
| 4-chlorophenyl | 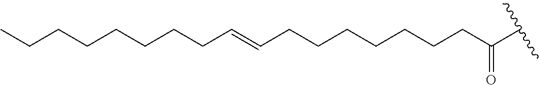 [SMILES: *C(=O)CCCCCCC/C=C/CCCCCCCC] |
| 4-chlorophenyl | 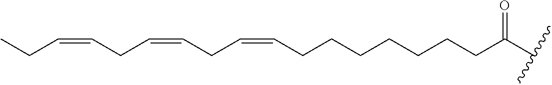 [SMILES: *C(=O)CCCCCCC/C=C\C/C=C\C/C=C\CC] |
| 4-chlorophenyl | tBu |
| 4-chlorophenyl | phenyl |
| 4-chlorophenyl | 4-chlorophenyl |
| 4-chlorophenyl | 3,4-dichlorophenyl |
| 4-chlorophenyl | 4-methylphenyl |
| 4-chlorophenyl | 4-methoxyphenyl |
| 3,4-dichlorophenyl | Me |
| 3,4-dichlorophenyl | —C₇H₁₅ |
| 3,4-dichlorophenyl | —C₉H₁₉ |
| 3,4-dichlorophenyl | —C₁₁H₂₃ |
| 3,4-dichlorophenyl | —C₁₃H₂₇ |
| 3,4-dichlorophenyl | —C₁₅H₃₁ |
| 3,4-dichlorophenyl | —C₁₇H₃₅ |
| 3,4-dichlorophenyl | 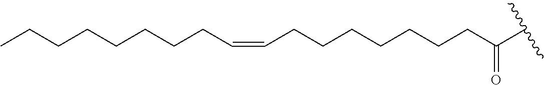 [SMILES: *C(=O)CCCCCCC/C=C\CCCCCCCC] |
| 3,4-dichlorophenyl | 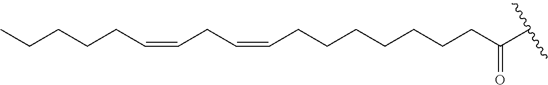 [SMILES: *C(=O)CCCCCCC/C=C\C/C=C\CCCCC] |
| 3,4-dichlorophenyl | 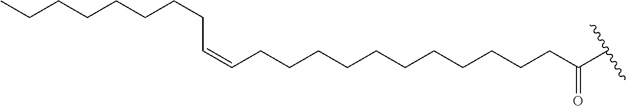 [SMILES: *C(=O)CCCCCCCCCCC/C=C\CCCCCCCC] |
| 3,4-dichlorophenyl | 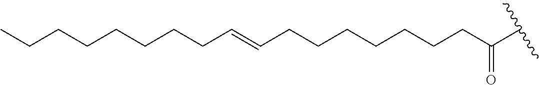 [SMILES: *C(=O)CCCCCCC/C=C/CCCCCCCC] |
| 3,4-dichlorophenyl | 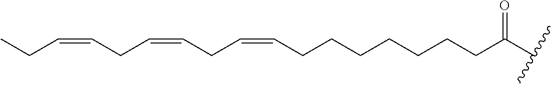 [SMILES: *C(=O)CCCCCCC/C=C\C/C=C\C/C=C\CC] |
| 3,4-dichlorophenyl | tBu |
| 3,4-dichlorophenyl | phenyl |
| 3,4-dichlorophenyl | 4-chlorophenyl |
| 3,4-dichlorophenyl | 3,4-dichlorophenyl |
| 3,4-dichlorophenyl | 4-methylphenyl |
| 3,4-dichlorophenyl | 4-methoxyphenyl |
| 4-methylphenyl | Me |
| 4-methylphenyl | —C₇H₁₅ |
| 4-methylphenyl | —C₉H₁₉ |
| 4-methylphenyl | —C₁₁H₂₃ |
| 4-methylphenyl | —C₁₃H₂₇ |
| 4-methylphenyl | —C₁₅H₃₁ |
| 4-methylphenyl | —C₁₇H₃₅ |
| 4-methylphenyl | 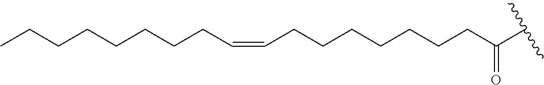 [SMILES: *C(=O)CCCCCCC/C=C\CCCCCCCC] |
| 4-methylphenyl | 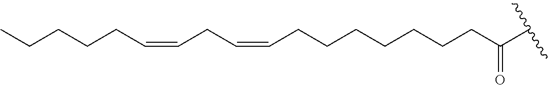 [SMILES: *C(=O)CCCCCCC/C=C\C/C=C\CCCCC] |
| 4-methylphenyl | 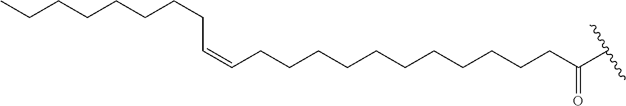 [SMILES: *C(=O)CCCCCCCCCCC/C=C\CCCCCCCC] |
| 4-methylphenyl | 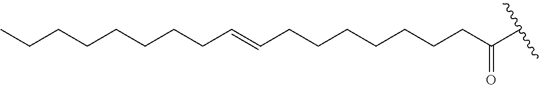 [SMILES: *C(=O)CCCCCCC/C=C/CCCCCCCC] |
| 4-methylphenyl | 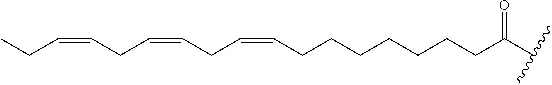 [SMILES: *C(=O)CCCCCCC/C=C\C/C=C\C/C=C\CC] |
| 4-methylphenyl | tBu |
| 4-methylphenyl | phenyl |
| 4-methylphenyl | 4-chlorophenyl |
| 4-methylphenyl | 3,4-dichlorophenyl |
| 4-methylphenyl | 4-methylphenyl |
| 4-methylphenyl | 4-methoxyphenyl |
| 4-methoxyphenyl | Me |
| 4-methoxyphenyl | —C₇H₁₅ |
| 4-methoxyphenyl | —C₉H₁₉ |
| 4-methoxyphenyl | —C₁₁H₂₃ |
| 4-methoxyphenyl | —C₁₃H₂₇ |
| 4-methoxyphenyl | —C₁₅H₃₁ |
| 4-methoxyphenyl | —C₁₇H₃₅ |
| 4-methoxyphenyl | 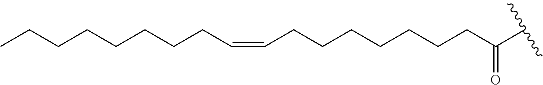 [SMILES: *C(=O)CCCCCCC/C=C\CCCCCCCC] |
| 4-methoxyphenyl | 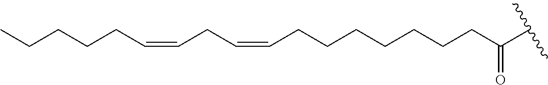 [SMILES: *C(=O)CCCCCCC/C=C\C/C=C\CCCCC] |
| 4-methoxyphenyl | 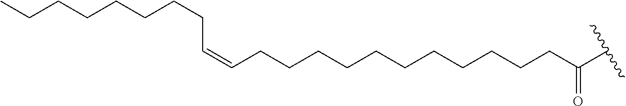 [SMILES: *C(=O)CCCCCCCCCCC/C=C\CCCCCCCC] |
| 4-methoxyphenyl | 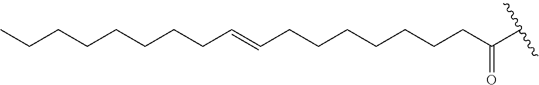 [SMILES: *C(=O)CCCCCCC/C=C/CCCCCCCC] |
| 4-methoxyphenyl | 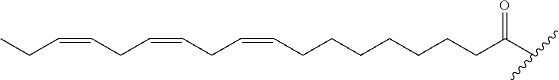 [SMILES: *C(=O)CCCCCCC/C=C\C/C=C\C/C=C\CC] |
| 4-methoxyphenyl | tBu |
| 4-methoxyphenyl | phenyl |
| 4-methoxyphenyl | 4-chlorophenyl |
| 4-methoxyphenyl | 3,4-dichlorophenyl |
| 4-methoxyphenyl | 4-methylphenyl |
| 4-methoxyphenyl | 4-methoxyphenyl |
| 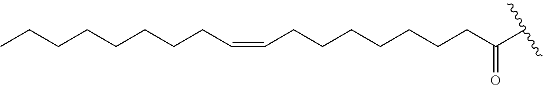 [SMILES: *C(=O)CCCCCCC/C=C\CCCCCCCC] | Me |
| 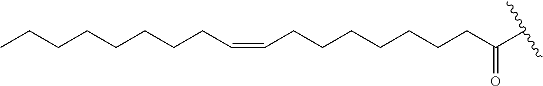 [SMILES: *C(=O)CCCCCCC/C=C\CCCCCCCC] | —C₇H₁₅ |
| 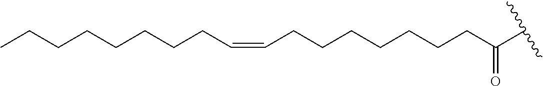 [SMILES: *C(=O)CCCCCCC/C=C\CCCCCCCC] | —C₉H₁₉ |
| 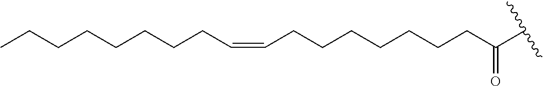 [SMILES: *C(=O)CCCCCCC/C=C\CCCCCCCC] | —C₁₁H₂₃ |
| 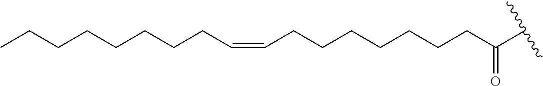 [SMILES: *C(=O)CCCCCCC/C=C\CCCCCCCC] | —C₁₃H₂₇ |
| 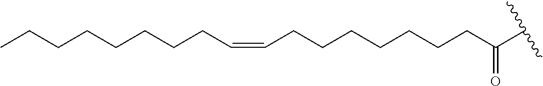 [SMILES: *C(=O)CCCCCCC/C=C\CCCCCCCC] | —C₁₅H₃₁ |
| 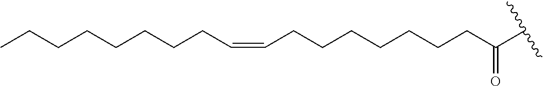 [SMILES: *C(=O)CCCCCCC/C=C\CCCCCCCC] | —C₁₇H₃₅ |
| 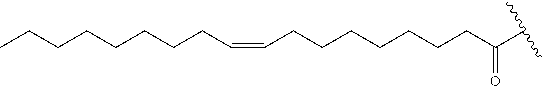 [SMILES: *C(=O)CCCCCCC/C=C\CCCCCCCC] | 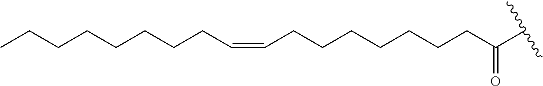 [SMILES: *C(=O)CCCCCCC/C=C\CCCCCCCC] |
| 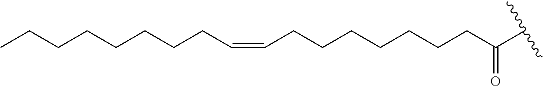 [SMILES: *C(=O)CCCCCCC/C=C\CCCCCCCC] | 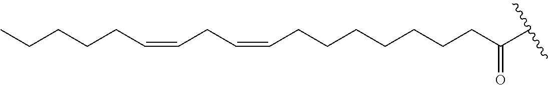 [SMILES: *C(=O)CCCCCCC/C=C\C/C=C\CCCCC] |
| 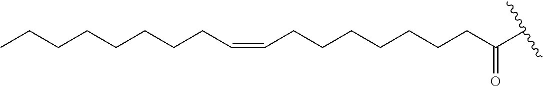 [SMILES: *C(=O)CCCCCCC/C=C\CCCCCCCC] | 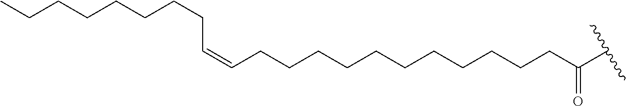 [SMILES: *C(=O)CCCCCCCCCCC/C=C\CCCCCCCC] |
| 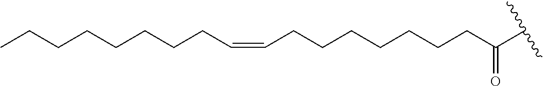 [SMILES: *C(=O)CCCCCCC/C=C\CCCCCCCC] | 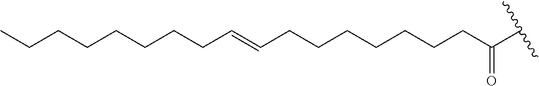 [SMILES: *C(=O)CCCCCCC/C=C/CCCCCCCC] |
| 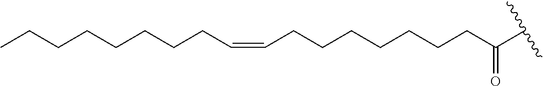 [SMILES: *C(=O)CCCCCCC/C=C\CCCCCCCC] | 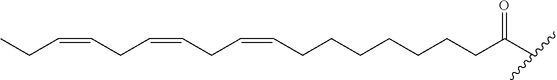 [SMILES: *C(=O)CCCCCCC/C=C\C/C=C\C/C=C\CC] |
| 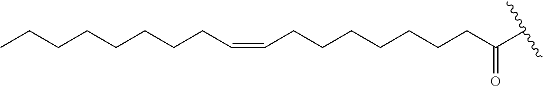 [SMILES: *C(=O)CCCCCCC/C=C\CCCCCCCC] | tBu |
| 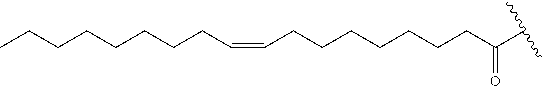 [SMILES: *C(=O)CCCCCCC/C=C\CCCCCCCC] | phenyl |
| 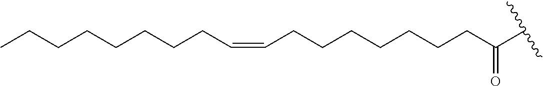 [SMILES: *C(=O)CCCCCCC/C=C\CCCCCCCC] | 4-chlorophenyl |
| 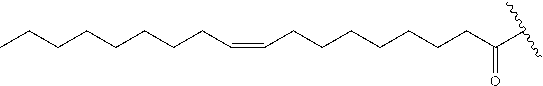 [SMILES: *C(=O)CCCCCCC/C=C\CCCCCCCC] | 3,4-dichlorophenyl |
| 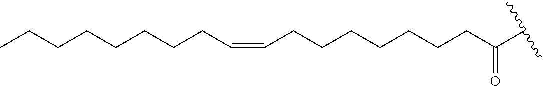 [SMILES: *C(=O)CCCCCCC/C=C\CCCCCCCC] | 4-methylphenyl |
| 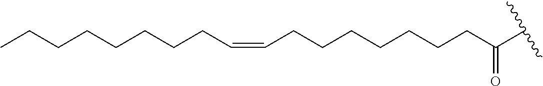 [SMILES: *C(=O)CCCCCCC/C=C\CCCCCCCC] | 4-methoxyphenyl |
| 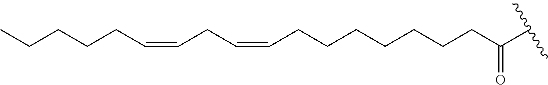 [SMILES: *C(=O)CCCCCCC/C=C\C/C=C\CCCCC] | Me |
| 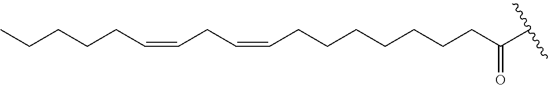 [SMILES: *C(=O)CCCCCCC/C=C\C/C=C\CCCCC] | —C₇H₁₅ |
| 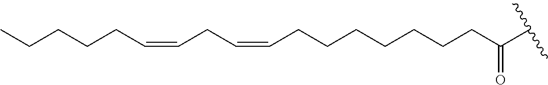 [SMILES: *C(=O)CCCCCCC/C=C\C/C=C\CCCCC] | —C₉H₁₉ |
| 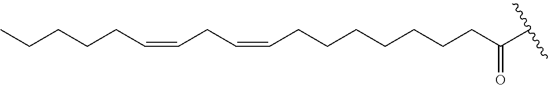 [SMILES: *C(=O)CCCCCCC/C=C\C/C=C\CCCCC] | —C₁₁H₂₃ |
| 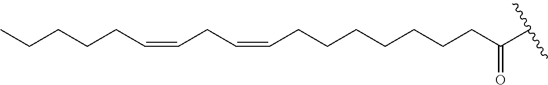 [SMILES: *C(=O)CCCCCCC/C=C\C/C=C\CCCCC] | —C₁₃H₂₇ |
| 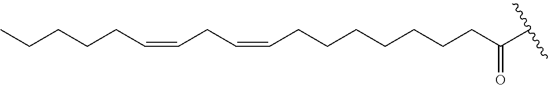 [SMILES: *C(=O)CCCCCCC/C=C\C/C=C\CCCCC] | —C₁₅H₃₁ |
| 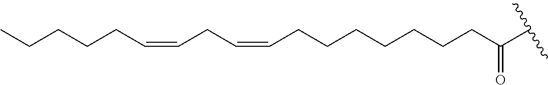 [SMILES: *C(=O)CCCCCCC/C=C\C/C=C\CCCCC] | —C₁₇H₃₅ |
| 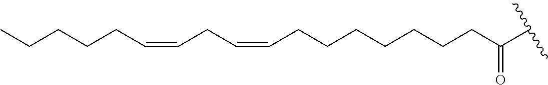 [SMILES: *C(=O)CCCCCCC/C=C\C/C=C\CCCCC] | 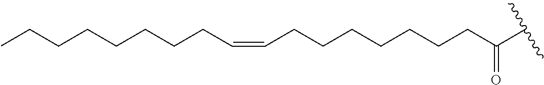 [SMILES: *C(=O)CCCCCCC/C=C\CCCCCCCC] |
| 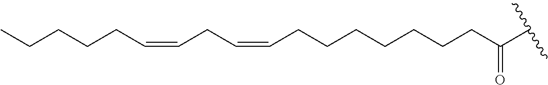 [SMILES: *C(=O)CCCCCCC/C=C\C/C=C\CCCCC] | 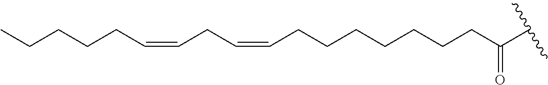 [SMILES: *C(=O)CCCCCCC/C=C\C/C=C\CCCCC] |
| 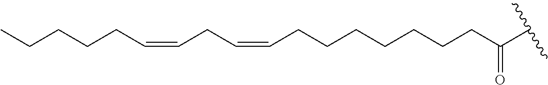 [SMILES: *C(=O)CCCCCCC/C=C\C/C=C\CCCCC] | 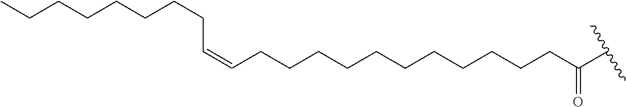 [SMILES: *C(=O)CCCCCCCCCCC/C=C\CCCCCCCC] |
| 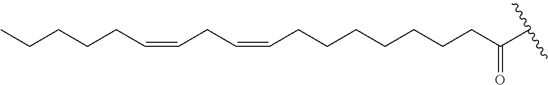 [SMILES: *C(=O)CCCCCCC/C=C\C/C=C\CCCCC] | 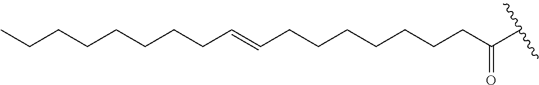 [SMILES: *C(=O)CCCCCCC/C=C/CCCCCCCC] |
| 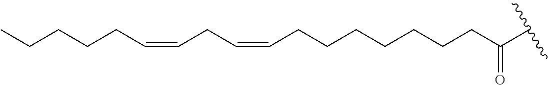 [SMILES: *C(=O)CCCCCCC/C=C\C/C=C\CCCCC] | 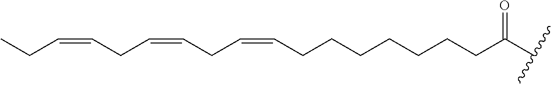 [SMILES: *C(=O)CCCCCCC/C=C\C/C=C\C/C=C\CC] |
| 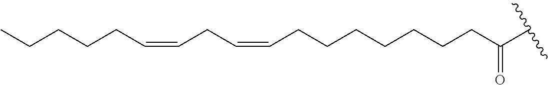 [SMILES: *C(=O)CCCCCCC/C=C\C/C=C\CCCCC] | tBu |
| 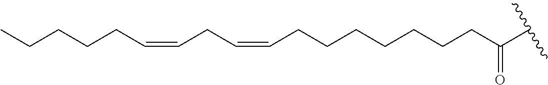 [SMILES: *C(=O)CCCCCCC/C=C\C/C=C\CCCCC] | phenyl |
| 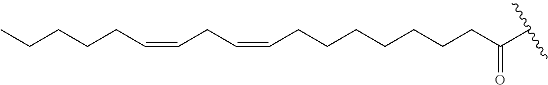 [SMILES: *C(=O)CCCCCCC/C=C\C/C=C\CCCCC] | 4-chlorophenyl |
| 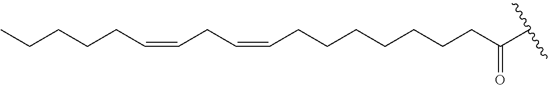 [SMILES: *C(=O)CCCCCCC/C=C\C/C=C\CCCCC] | 3,4-dichlorophenyl |
| 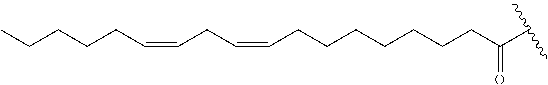 [SMILES: *C(=O)CCCCCCC/C=C\C/C=C\CCCCC] | 4-methylphenyl |
| 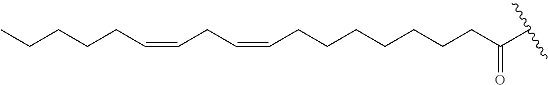 [SMILES: *C(=O)CCCCCCC/C=C\C/C=C\CCCCC] | 4-methoxyphenyl |
| 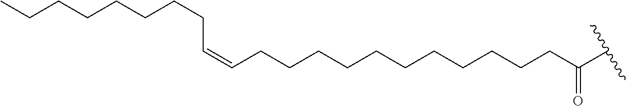 [SMILES: *C(=O)CCCCCCCCCCC/C=C\CCCCCCCC] | Me |
| 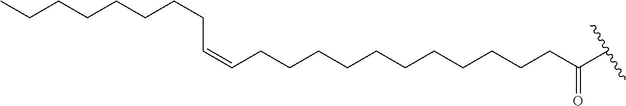 [SMILES: *C(=O)CCCCCCCCCCC/C=C\CCCCCCCC] | —C₇H₁₅ |
| 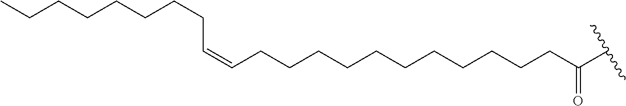 [SMILES: *C(=O)CCCCCCCCCCC/C=C\CCCCCCCC] | —C₉H₁₉ |
| 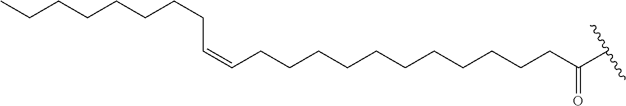 [SMILES: *C(=O)CCCCCCCCCCC/C=C\CCCCCCCC] | —C₁₁H₂₃ |
| 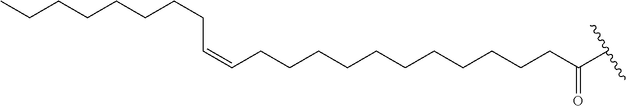 [SMILES: *C(=O)CCCCCCCCCCC/C=C\CCCCCCCC] | —C₁₃H₂₇ |
| 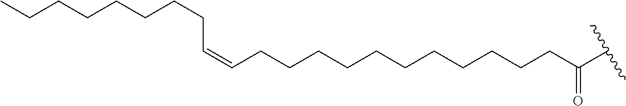 [SMILES: *C(=O)CCCCCCCCCCC/C=C\CCCCCCCC] | —C₁₅H₃₁ |
| 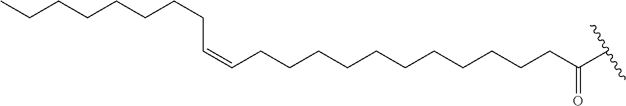 [SMILES: *C(=O)CCCCCCCCCCC/C=C\CCCCCCCC] | —C₁₇H₃₅ |
| 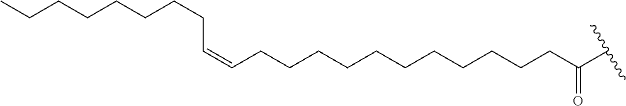 [SMILES: *C(=O)CCCCCCCCCCC/C=C\CCCCCCCC] | 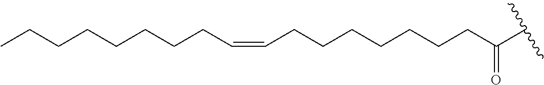 [SMILES: *C(=O)CCCCCCC/C=C\CCCCCCCC] |
| 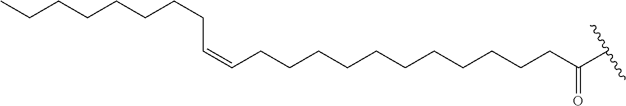 [SMILES: *C(=O)CCCCCCCCCCC/C=C\CCCCCCCC] | 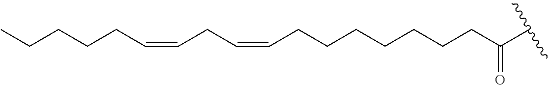 [SMILES: *C(=O)CCCCCCC/C=C\C/C=C\CCCCC] |
| 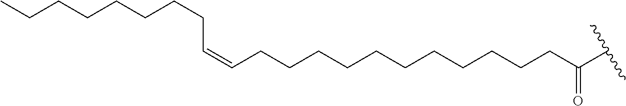 [SMILES: *C(=O)CCCCCCCCCCC/C=C\CCCCCCCC] | 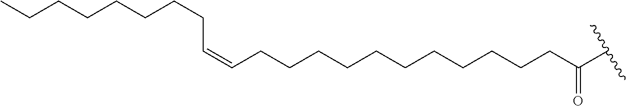 [SMILES: *C(=O)CCCCCCCCCCC/C=C\CCCCCCCC] |
| 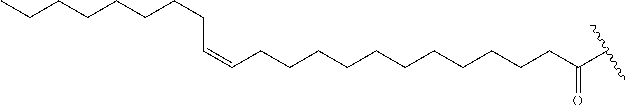 [SMILES: *C(=O)CCCCCCCCCCC/C=C\CCCCCCCC] | 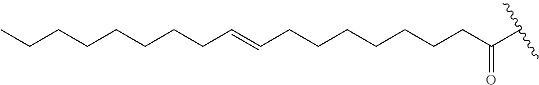 [SMILES: *C(=O)CCCCCCC/C=C/CCCCCCCC] |
| 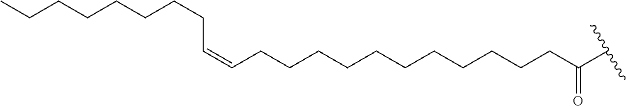 [SMILES: *C(=O)CCCCCCCCCCC/C=C\CCCCCCCC] | 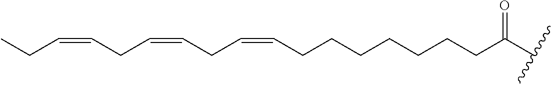 [SMILES: *C(=O)CCCCCCC/C=C\C/C=C\C/C=C\CC] |
| 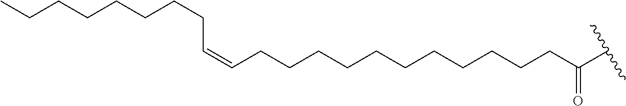 [SMILES: *C(=O)CCCCCCCCCCC/C=C\CCCCCCCC] | tBu |
| 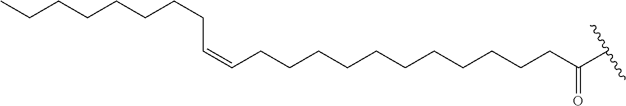 [SMILES: *C(=O)CCCCCCCCCCC/C=C\CCCCCCCC] | phenyl |
| 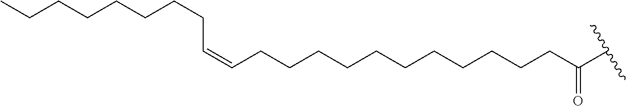 [SMILES: *C(=O)CCCCCCCCCCC/C=C\CCCCCCCC] | 4-chlorophenyl |
| 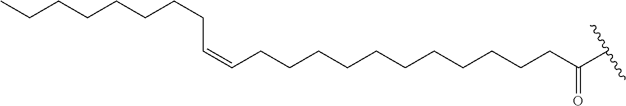 [SMILES: *C(=O)CCCCCCCCCCC/C=C\CCCCCCCC] | 3,4-dichlorophenyl |
| 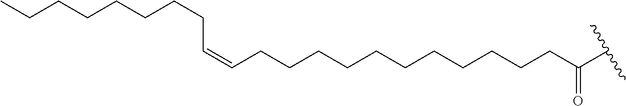 [SMILES: *C(=O)CCCCCCCCCCC/C=C\CCCCCCCC] | 4-methylphenyl |
| 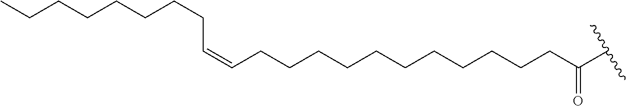 [SMILES: *C(=O)CCCCCCCCCCC/C=C\CCCCCCCC] | 4-methoxyphenyl |
| 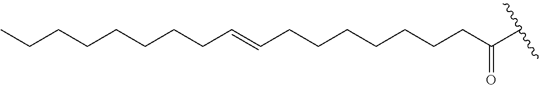 [SMILES: *C(=O)CCCCCCC/C=C/CCCCCCCC] | Me |
| 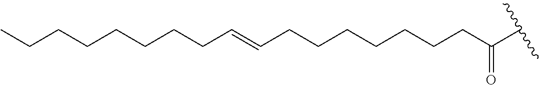 [SMILES: *C(=O)CCCCCCC/C=C/CCCCCCCC] | —C₇H₁₅ |
| 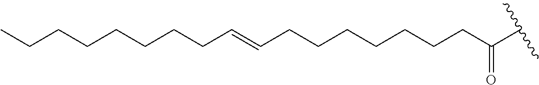 [SMILES: *C(=O)CCCCCCC/C=C/CCCCCCCC] | —C₉H₁₉ |
| 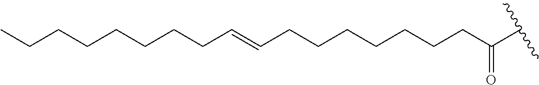 [SMILES: *C(=O)CCCCCCC/C=C/CCCCCCCC] | —C₁₁H₂₃ |
| 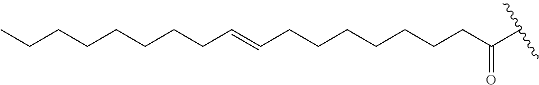 [SMILES: *C(=O)CCCCCCC/C=C/CCCCCCCC] | —C₁₃H₂₇ |
| 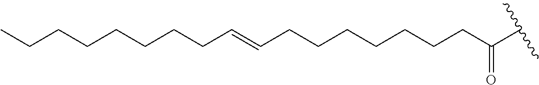 [SMILES: *C(=O)CCCCCCC/C=C/CCCCCCCC] | —C₁₅H₃₁ |
| 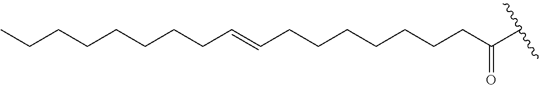 [SMILES: *C(=O)CCCCCCC/C=C/CCCCCCCC] | —C₁₇H₃₅ |
| 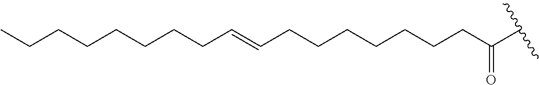 [SMILES: *C(=O)CCCCCCC/C=C/CCCCCCCC] | 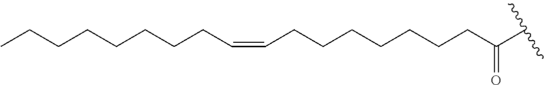 [SMILES: *C(=O)CCCCCCC/C=C\CCCCCCCC] |
| 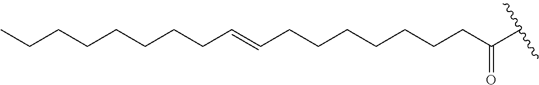 [SMILES: *C(=O)CCCCCCC/C=C/CCCCCCCC] | 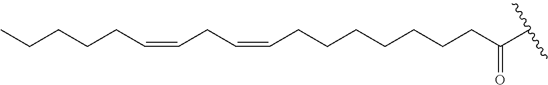 [SMILES: *C(=O)CCCCCCC/C=C\C/C=C\CCCCC] |
| 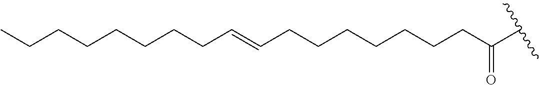 [SMILES: *C(=O)CCCCCCC/C=C/CCCCCCCC] | 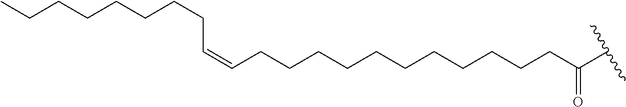 [SMILES: *C(=O)CCCCCCCCCCC/C=C\CCCCCCCC] |
| 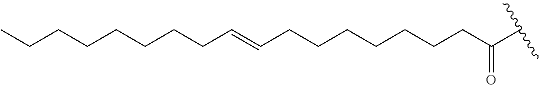 [SMILES: *C(=O)CCCCCCC/C=C/CCCCCCCC] | 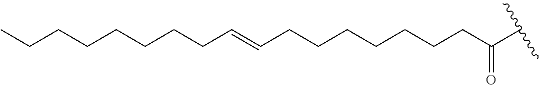 [SMILES: *C(=O)CCCCCCC/C=C/CCCCCCCC] |
| 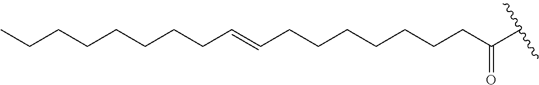 [SMILES: *C(=O)CCCCCCC/C=C/CCCCCCCC] | 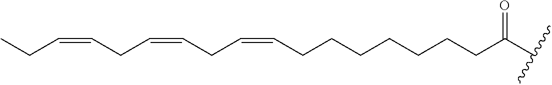 [SMILES: *C(=O)CCCCCCC/C=C\C/C=C\C/C=C\CC] |
| 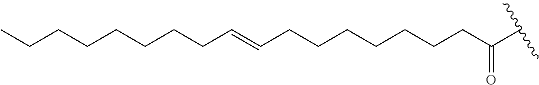 [SMILES: *C(=O)CCCCCCC/C=C/CCCCCCCC] | tBu |
| 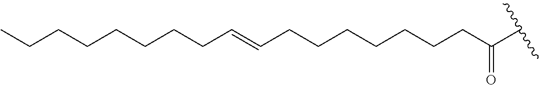 [SMILES: *C(=O)CCCCCCC/C=C/CCCCCCCC] | phenyl |
| 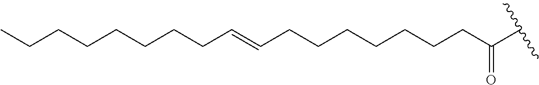 [SMILES: *C(=O)CCCCCCC/C=C/CCCCCCCC] | 4-chlorophenyl |
| 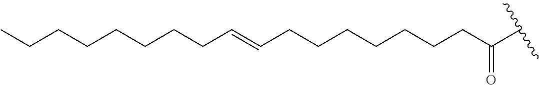 [SMILES: *C(=O)CCCCCCC/C=C/CCCCCCCC] | 3,4-dichlorophenyl |
| 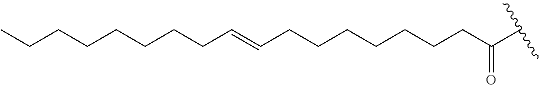 [SMILES: *C(=O)CCCCCCC/C=C/CCCCCCCC] | 4-methylphenyl |
| 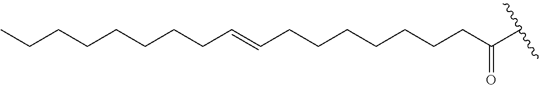 [SMILES: *C(=O)CCCCCCC/C=C/CCCCCCCC] | 4-methoxyphenyl |
| 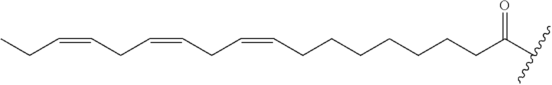 [SMILES: *C(=O)CCCCCCC/C=C\C/C=C\C/C=C\CC] | Me |
| 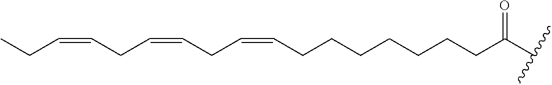 [SMILES: *C(=O)CCCCCCC/C=C\C/C=C\C/C=C\CC] | —C₇H₁₅ |
| 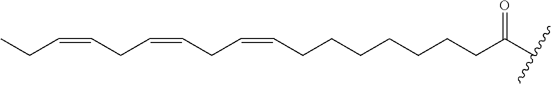 [SMILES: *C(=O)CCCCCCC/C=C\C/C=C\C/C=C\CC] | —C₉H₁₉ |
| 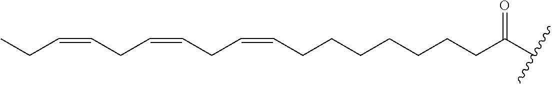 [SMILES: *C(=O)CCCCCCC/C=C\C/C=C\C/C=C\CC] | —C₁₁H₂₃ |
| 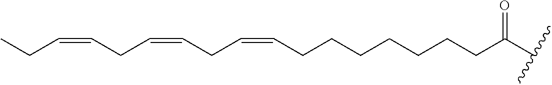 [SMILES: *C(=O)CCCCCCC/C=C\C/C=C\C/C=C\CC] | —C₁₃H₂₇ |
| 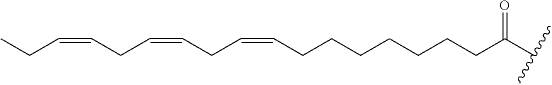 [SMILES: *C(=O)CCCCCCC/C=C\C/C=C\C/C=C\CC] | —C₁₅H₃₁ |
| 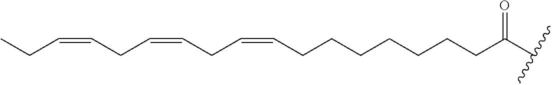 [SMILES: *C(=O)CCCCCCC/C=C\C/C=C\C/C=C\CC] | —C₁₇H₃₅ |
| 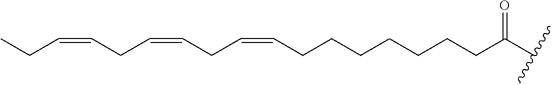 [SMILES: *C(=O)CCCCCCC/C=C\C/C=C\C/C=C\CC] | 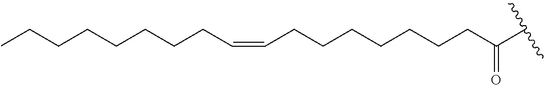 [SMILES: *C(=O)CCCCCCC/C=C\CCCCCCCC] |
| 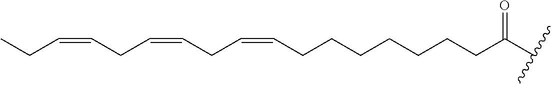 [SMILES: *C(=O)CCCCCCC/C=C\C/C=C\C/C=C\CC] | 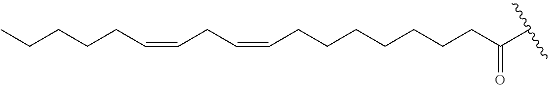 [SMILES: *C(=O)CCCCCCC/C=C\C/C=C\CCCCC] |
| 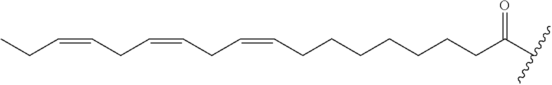 [SMILES: *C(=O)CCCCCCC/C=C\C/C=C\C/C=C\CC] | 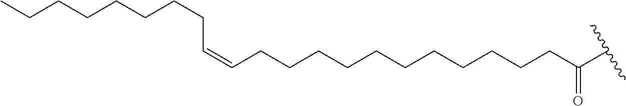 [SMILES: *C(=O)CCCCCCCCCCC/C=C\CCCCCCCC] |
| 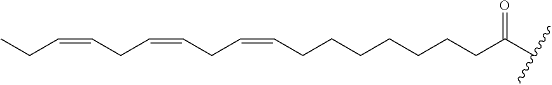 [SMILES: *C(=O)CCCCCCC/C=C\C/C=C\C/C=C\CC] | 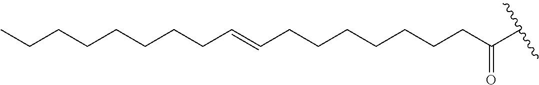 [SMILES: *C(=O)CCCCCCC/C=C/CCCCCCCC] |
| 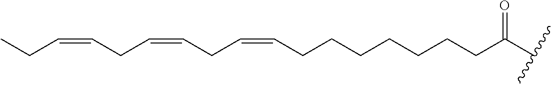 [SMILES: *C(=O)CCCCCCC/C=C\C/C=C\C/C=C\CC] | 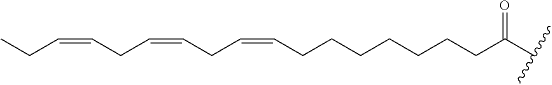 [SMILES: *C(=O)CCCCCCC/C=C\C/C=C\C/C=C\CC] |
| 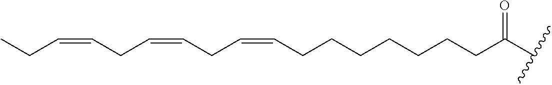 [SMILES: *C(=O)CCCCCCC/C=C\C/C=C\C/C=C\CC] | tBu |
| 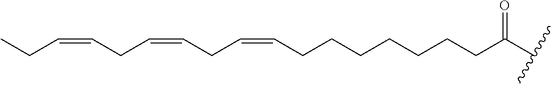 [SMILES: *C(=O)CCCCCCC/C=C\C/C=C\C/C=C\CC] | phenyl |
| 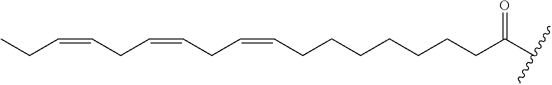 [SMILES: *C(=O)CCCCCCC/C=C\C/C=C\C/C=C\CC] | 4-chlorophenyl |
| 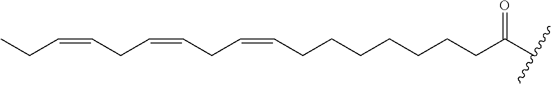 [SMILES: *C(=O)CCCCCCC/C=C\C/C=C\C/C=C\CC] | 3,4-dichlorophenyl |
| 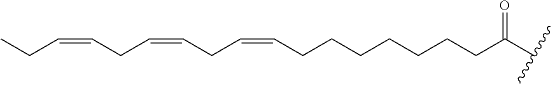 [SMILES: *C(=O)CCCCCCC/C=C\C/C=C\C/C=C\CC] | 4-methylphenyl |
| 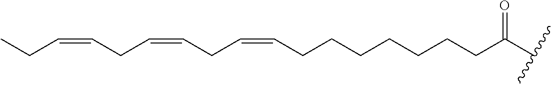 [SMILES: *C(=O)CCCCCCC/C=C\C/C=C\C/C=C\CC] | 4-methoxyphenyl |
| H | 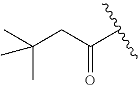 [SMILES: *C(=O)CC(C)(C)C] |
| H | 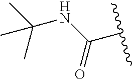 [SMILES: *C(=O)NC(C)(C)C] |
| H | 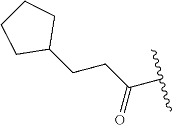 [SMILES: *C(=O)CCC1CCCC1] |
| H | 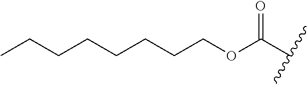 [SMILES: *C(=O)OCCCCCCCC] |
| H | 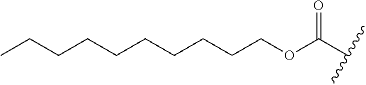 [SMILES: *C(=O)OCCCCCCCCCC] |
| H | 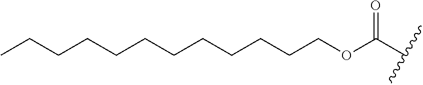 [SMILES: *C(=O)OCCCCCCCCCCCC] |
| H | 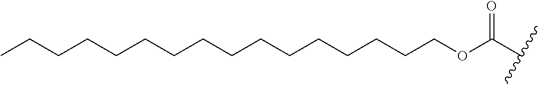 [SMILES: *C(=O)OCCCCCCCCCCCCCCCC] |
| H | 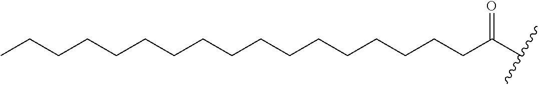 [SMILES: *C(=O)CCCCCCCCCCCCCCCCC] |
| H | 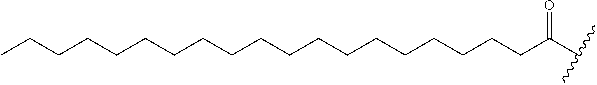 [SMILES: *C(=O)CCCCCCCCCCCCCCCCCCC] |
| H | 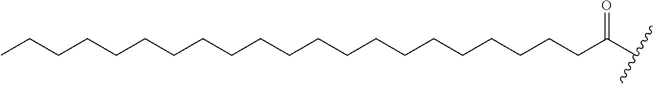 [SMILES: *C(=O)CCCCCCCCCCCCCCCCCCCCC] |
| H | 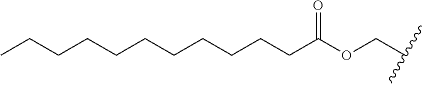 [SMILES: *COC(=O)CCCCCCCCCCC] |
| H | 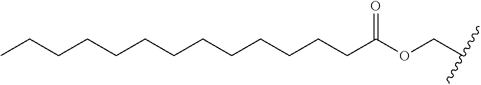 [SMILES: *COC(=O)CCCCCCCCCCCCC] |
| H | 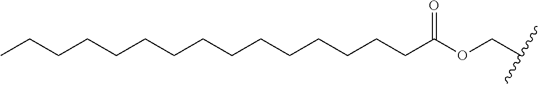 [SMILES: *COC(=O)CCCCCCCCCCCCCCC] |
| H | 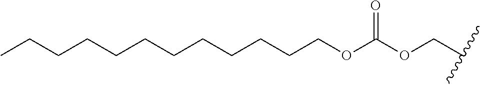 [SMILES: *COC(=O)OCCCCCCCCCCCC] |
| H | 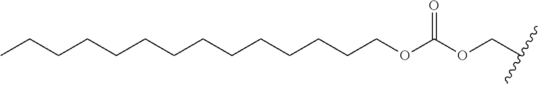 [SMILES: *COC(=O)OCCCCCCCCCCCCCC] |
| H | 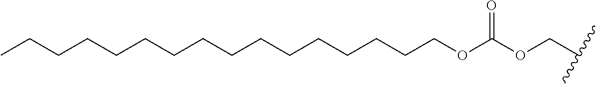 [SMILES: *COC(=O)OCCCCCCCCCCCCCCCC] |
| Me | 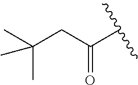 [SMILES: *C(=O)CC(C)(C)C] |
| Me | 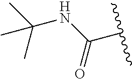 [SMILES: *C(=O)NC(C)(C)C] |
| Me | 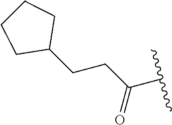 [SMILES: *C(=O)CCC1CCCC1] |
| Me | 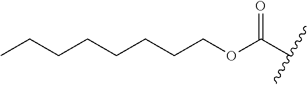 [SMILES: *C(=O)OCCCCCCCC] |
| Me | 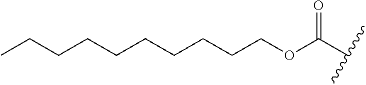 [SMILES: *C(=O)OCCCCCCCCCC] |
| Me | 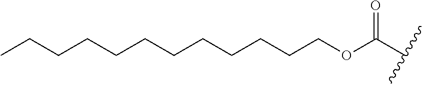 [SMILES: *C(=O)OCCCCCCCCCCCC] |
| Me | 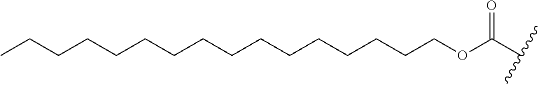 [SMILES: *C(=O)OCCCCCCCCCCCCCCCC] |
| Me | 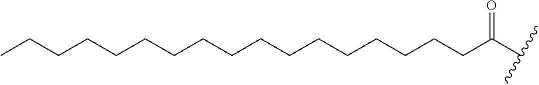 [SMILES: *C(=O)CCCCCCCCCCCCCCCCC] |
| Me | 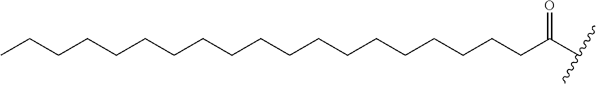 [SMILES: *C(=O)CCCCCCCCCCCCCCCCCCC] |
| Me | 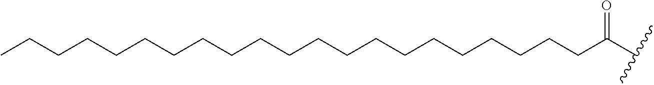 [SMILES: *C(=O)CCCCCCCCCCCCCCCCCCCCC] |
| Me | 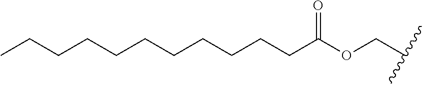 [SMILES: *COC(=O)CCCCCCCCCCC] |
| Me | 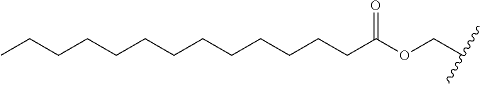 [SMILES: *COC(=O)CCCCCCCCCCCCC] |
| Me | 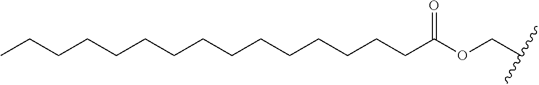 [SMILES: *COC(=O)CCCCCCCCCCCCCCC] |
| Me | 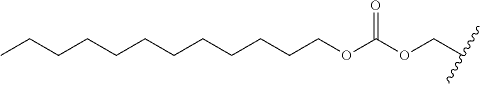 [SMILES: *COC(=O)OCCCCCCCCCCCC] |
| Me | 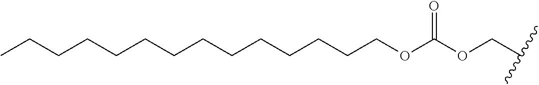 [SMILES: *COC(=O)OCCCCCCCCCCCCCC] |
| Me | 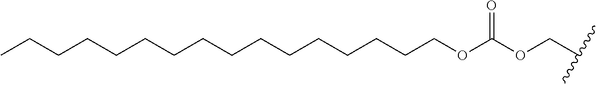 [SMILES: *COC(=O)OCCCCCCCCCCCCCCCC] |
| —C₇H₁₅ | 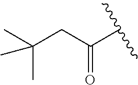 [SMILES: *C(=O)CC(C)(C)C] |
| —C₇H₁₅ | 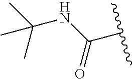 [SMILES: *C(=O)NC(C)(C)C] |
| —C₇H₁₅ | 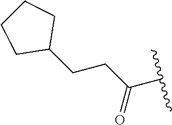 [SMILES: *C(=O)CCC1CCCC1] |
| —C₇H₁₅ | 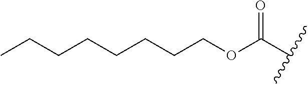 [SMILES: *C(=O)OCCCCCCCC] |
| —C₇H₁₅ | 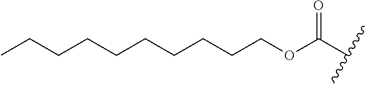 [SMILES: *C(=O)OCCCCCCCCCC] |
| —C₇H₁₅ | 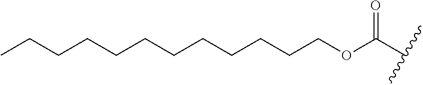 [SMILES: *C(=O)OCCCCCCCCCCCC] |
| —C₇H₁₅ | 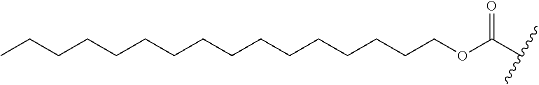 [SMILES: *C(=O)OCCCCCCCCCCCCCCCC] |
| —C₇H₁₅ | 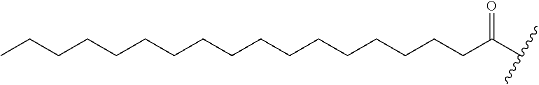 [SMILES: *C(=O)CCCCCCCCCCCCCCCCC] |
| —C₇H₁₅ | 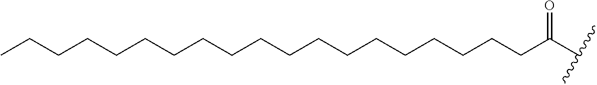 [SMILES: *C(=O)CCCCCCCCCCCCCCCCCCC] |
| —C₇H₁₅ | 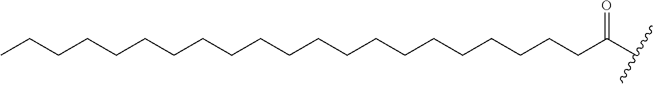 [SMILES: *C(=O)CCCCCCCCCCCCCCCCCCCCC] |
| —C₇H₁₅ | 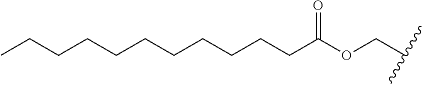 [SMILES: *COC(=O)CCCCCCCCCCC] |
| —C₇H₁₅ | 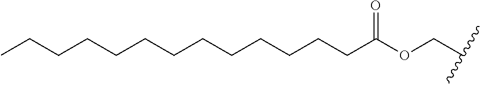 [SMILES: *COC(=O)CCCCCCCCCCCCC] |
| —C₇H₁₅ | 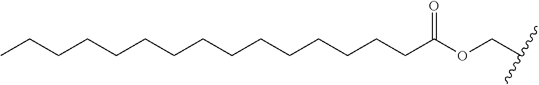 [SMILES: *COC(=O)CCCCCCCCCCCCCCC] |
| —C₇H₁₅ | 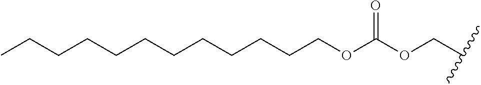 [SMILES: *COC(=O)OCCCCCCCCCCCC] |
| —C₇H₁₅ | 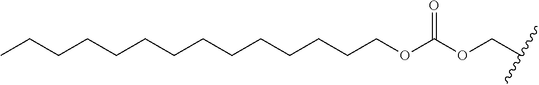 [SMILES: *COC(=O)OCCCCCCCCCCCCCC] |
| —C₇H₁₅ | 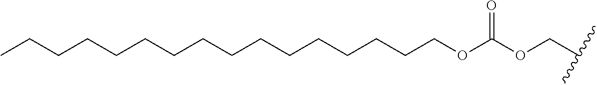 [SMILES: *COC(=O)OCCCCCCCCCCCCCCCC] |
| —C₉H₁₉ | 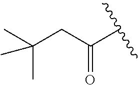 [SMILES: *C(=O)CC(C)(C)C] |
| —C₉H₁₉ | 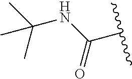 [SMILES: *C(=O)NC(C)(C)C] |
| —C₉H₁₉ | 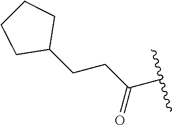 [SMILES: *C(=O)CCC1CCCC1] |
| —C₉H₁₉ | 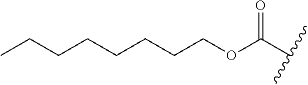 [SMILES: *C(=O)OCCCCCCCC] |
| —C₉H₁₉ | 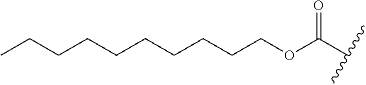 [SMILES: *C(=O)OCCCCCCCCCC] |
| —C₉H₁₉ | 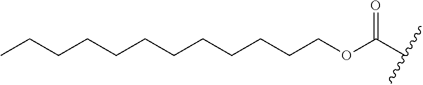 [SMILES: *C(=O)OCCCCCCCCCCCC] |
| —C₉H₁₉ | 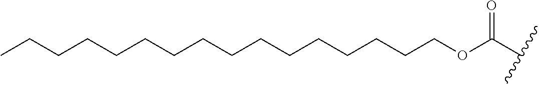 [SMILES: *C(=O)OCCCCCCCCCCCCCCCC] |
| —C₉H₁₉ | 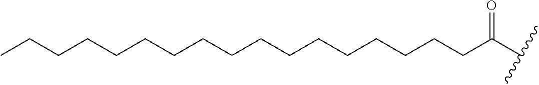 [SMILES: *C(=O)CCCCCCCCCCCCCCCCC] |
| —C₉H₁₉ | 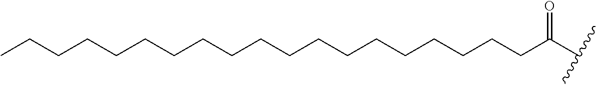 [SMILES: *C(=O)CCCCCCCCCCCCCCCCCCC] |
| —C₉H₁₉ | 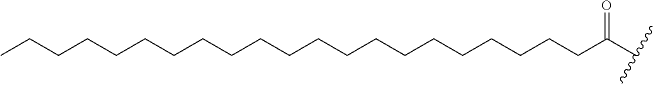 [SMILES: *C(=O)CCCCCCCCCCCCCCCCCCCCC] |
| —C₉H₁₉ | 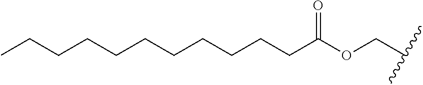 [SMILES: *COC(=O)CCCCCCCCCCC] |
| —C₉H₁₉ | 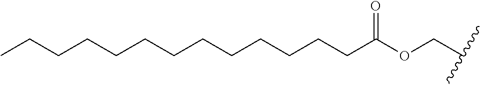 [SMILES: *COC(=O)CCCCCCCCCCCCC] |
| —C₉H₁₉ | 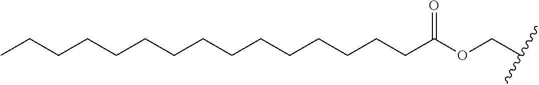 [SMILES: *COC(=O)CCCCCCCCCCCCCCC] |
| —C₉H₁₉ | 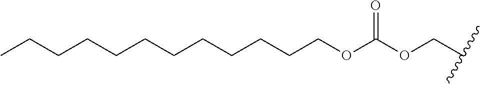 [SMILES: *COC(=O)OCCCCCCCCCCCC] |
| —C₉H₁₉ | 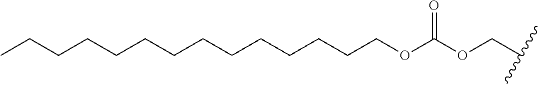 [SMILES: *COC(=O)OCCCCCCCCCCCCCC] |
| —C₉H₁₉ | 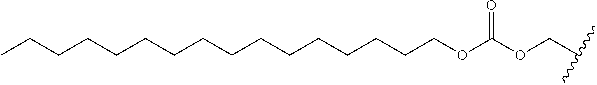 [SMILES: *COC(=O)OCCCCCCCCCCCCCCCC] |
| —C₁₁H₂₃ | 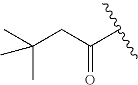 [SMILES: *C(=O)CC(C)(C)C] |
| —C₁₁H₂₃ | 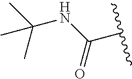 [SMILES: *C(=O)NC(C)(C)C] |
| —C₁₁H₂₃ | 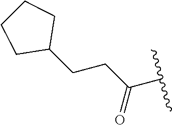 [SMILES: *C(=O)CCC1CCCC1] |
| —C₁₁H₂₃ | 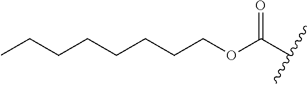 [SMILES: *C(=O)OCCCCCCCC] |
| —C₁₁H₂₃ | 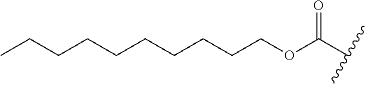 [SMILES: *C(=O)OCCCCCCCCCC] |
| —C₁₁H₂₃ | 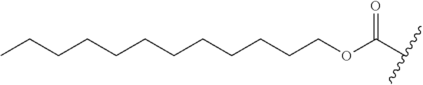 [SMILES: *C(=O)OCCCCCCCCCCCC] |
| —C₁₁H₂₃ | 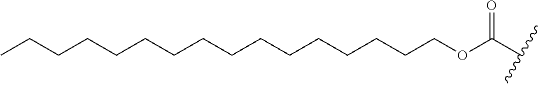 [SMILES: *C(=O)OCCCCCCCCCCCCCCCC] |
| —C₁₁H₂₃ | 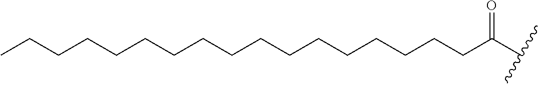 [SMILES: *C(=O)CCCCCCCCCCCCCCCCC] |
| —C₁₁H₂₃ | 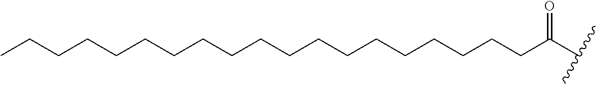 [SMILES: *C(=O)CCCCCCCCCCCCCCCCCCC] |
| —C₁₁H₂₃ | 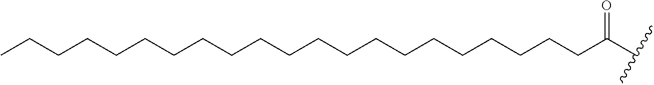 [SMILES: *C(=O)CCCCCCCCCCCCCCCCCCCCC] |
| —C₁₁H₂₃ | 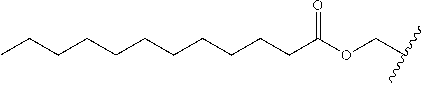 [SMILES: *COC(=O)CCCCCCCCCCC] |
| —C₁₁H₂₃ | 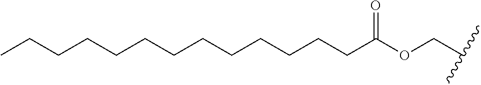 [SMILES: *COC(=O)CCCCCCCCCCCCC] |
| —C₁₁H₂₃ | 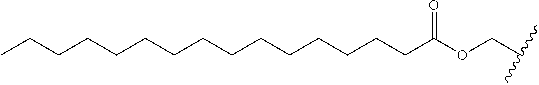 [SMILES: *COC(=O)CCCCCCCCCCCCCCC] |
| —C₁₁H₂₃ | 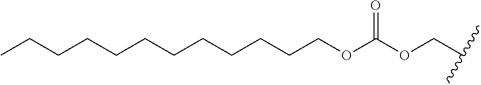 [SMILES: *COC(=O)OCCCCCCCCCCCC] |
| —C₁₁H₂₃ | 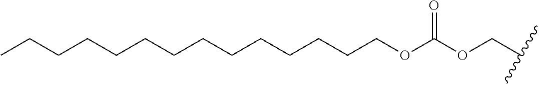 [SMILES: *COC(=O)OCCCCCCCCCCCCCC] |
| —C₁₁H₂₃ | 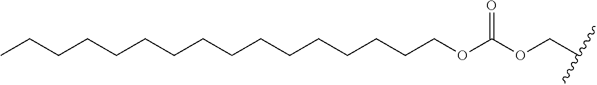 [SMILES: *COC(=O)OCCCCCCCCCCCCCCCC] |
| —C₁₃H₂₇ | 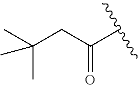 [SMILES: *C(=O)CC(C)(C)C] |
| —C₁₃H₂₇ | 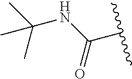 [SMILES: *C(=O)NC(C)(C)C] |
| —C₁₃H₂₇ | 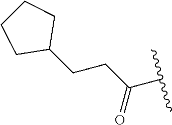 [SMILES: *C(=O)CCC1CCCC1] |
| —C₁₃H₂₇ | 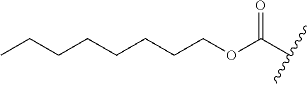 [SMILES: *C(=O)OCCCCCCCC] |
| —C₁₃H₂₇ | 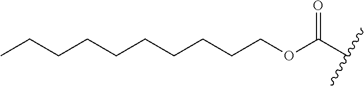 [SMILES: *C(=O)OCCCCCCCCCC] |
| —C₁₃H₂₇ | 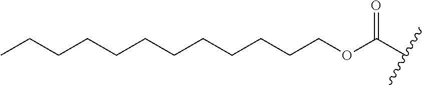 [SMILES: *C(=O)OCCCCCCCCCCCC] |
| —C₁₃H₂₇ | 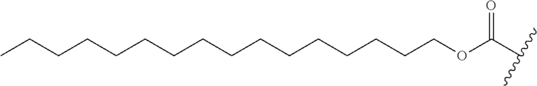 [SMILES: *C(=O)OCCCCCCCCCCCCCCCC] |
| —C₁₃H₂₇ | 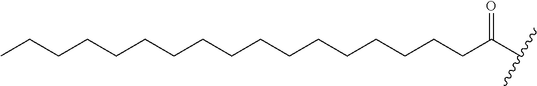 [SMILES: *C(=O)CCCCCCCCCCCCCCCCC] |
| —C₁₃H₂₇ | 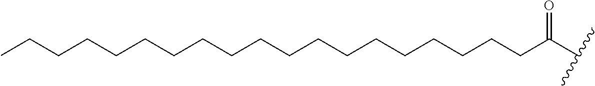 [SMILES: *C(=O)CCCCCCCCCCCCCCCCCCC] |
| —C₁₃H₂₇ | 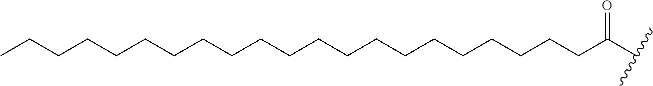 [SMILES: *C(=O)CCCCCCCCCCCCCCCCCCCCC] |
| —C₁₃H₂₇ | 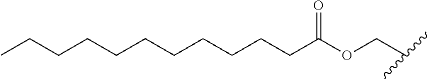 [SMILES: *COC(=O)CCCCCCCCCCC] |
| —C₁₃H₂₇ | 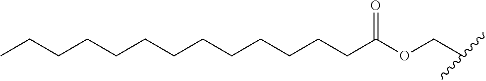 [SMILES: *COC(=O)CCCCCCCCCCCCC] |
| —C₁₃H₂₇ | 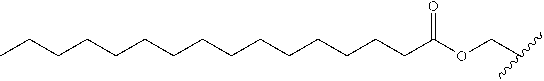 [SMILES: *COC(=O)CCCCCCCCCCCCCCC] |
| —C₁₃H₂₇ | 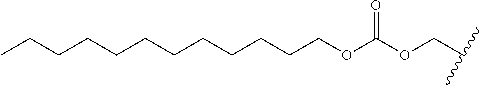 [SMILES: *COC(=O)OCCCCCCCCCCCC] |
| —C₁₃H₂₇ | 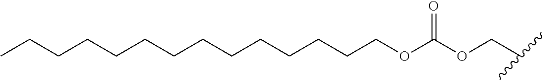 [SMILES: *COC(=O)OCCCCCCCCCCCCCC] |
| —C₁₃H₂₇ | 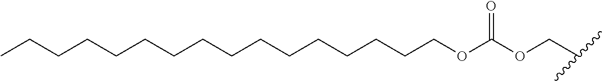 [SMILES: *COC(=O)OCCCCCCCCCCCCCCCC] |
| —C₁₅H₃₁ | 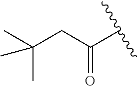 [SMILES: *C(=O)CC(C)(C)C] |
| —C₁₅H₃₁ | 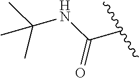 [SMILES: *C(=O)NC(C)(C)C] |
| —C₁₅H₃₁ | 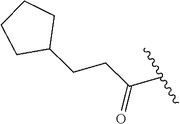 [SMILES: *C(=O)CCC1CCCC1] |
| —C₁₅H₃₁ | 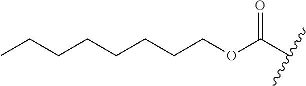 [SMILES: *C(=O)OCCCCCCCC] |
| —C₁₅H₃₁ | 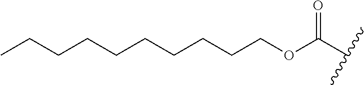 [SMILES: *C(=O)OCCCCCCCCCC] |
| —C₁₅H₃₁ | 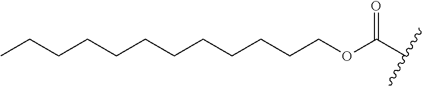 [SMILES: *C(=O)OCCCCCCCCCCCC] |
| —C₁₅H₃₁ | 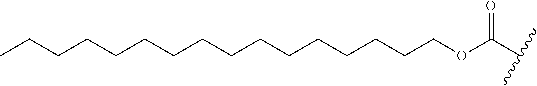 [SMILES: *C(=O)OCCCCCCCCCCCCCCCC] |
| —C₁₅H₃₁ | 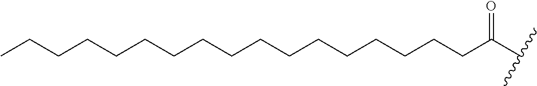 [SMILES: *C(=O)CCCCCCCCCCCCCCCCC] |
| —C₁₅H₃₁ | 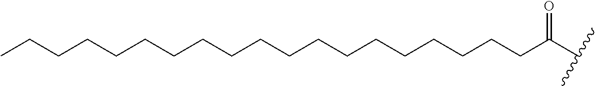 [SMILES: *C(=O)CCCCCCCCCCCCCCCCCCC] |
| —C₁₅H₃₁ | 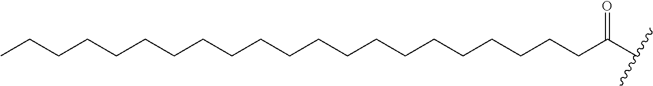 [SMILES: *C(=O)CCCCCCCCCCCCCCCCCCCCC] |
| —C₁₅H₃₁ | 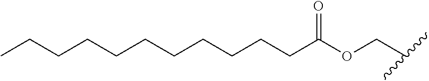 [SMILES: *COC(=O)CCCCCCCCCCC] |
| —C₁₅H₃₁ | 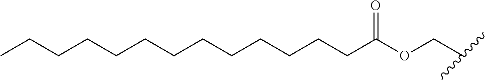 [SMILES: *COC(=O)CCCCCCCCCCCCC] |
| —C₁₅H₃₁ | 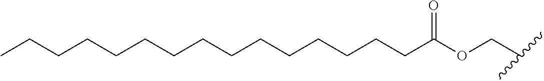 [SMILES: *COC(=O)CCCCCCCCCCCCCCC] |
| —C₁₅H₃₁ | 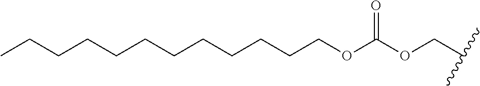 [SMILES: *COC(=O)OCCCCCCCCCCCC] |
| —C₁₅H₃₁ | 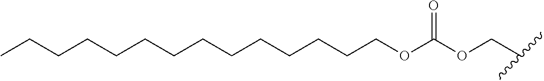 [SMILES: *COC(=O)OCCCCCCCCCCCCCC] |
| —C₁₅H₃₁ | 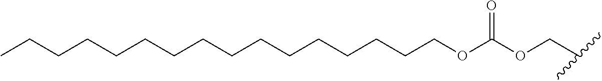 [SMILES: *COC(=O)OCCCCCCCCCCCCCCCC] |
| —C₁₇H₃₅ | 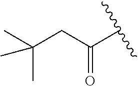 [SMILES: *C(=O)CC(C)(C)C] |
| —C₁₇H₃₅ | 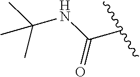 [SMILES: *C(=O)NC(C)(C)C] |
| —C₁₇H₃₅ | 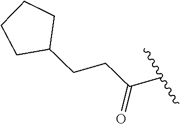 [SMILES: *C(=O)CCC1CCCC1] |
| —C₁₇H₃₅ | 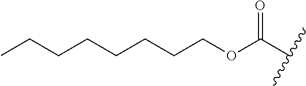 [SMILES: *C(=O)OCCCCCCCC] |
| —C₁₇H₃₅ | 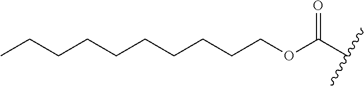 [SMILES: *C(=O)OCCCCCCCCCC] |
| —C₁₇H₃₅ | 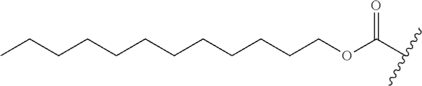 [SMILES: *C(=O)OCCCCCCCCCCCC] |
| —C₁₇H₃₅ | 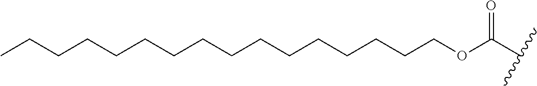 [SMILES: *C(=O)OCCCCCCCCCCCCCCCC] |
| —C₁₇H₃₅ | 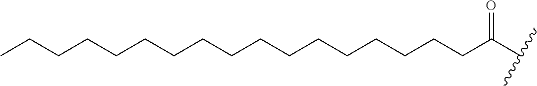 [SMILES: *C(=O)CCCCCCCCCCCCCCCCC] |
| —C₁₇H₃₅ | 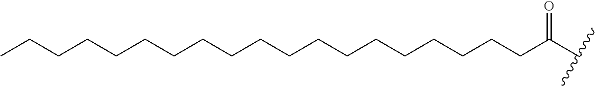 [SMILES: *C(=O)CCCCCCCCCCCCCCCCCCC] |
| —C₁₇H₃₅ | 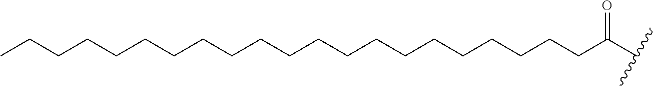 [SMILES: *C(=O)CCCCCCCCCCCCCCCCCCCCC] |
| —C₁₇H₃₅ | 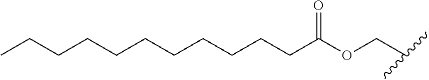 [SMILES: *COC(=O)CCCCCCCCCCC] |
| —C₁₇H₃₅ | 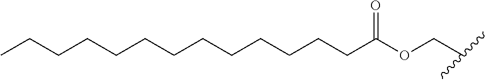 [SMILES: *COC(=O)CCCCCCCCCCCCC] |
| —C₁₇H₃₅ | 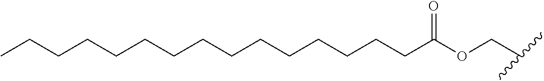 [SMILES: *COC(=O)CCCCCCCCCCCCCCC] |
| —C₁₇H₃₅ | 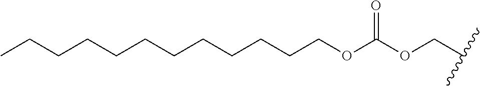 [SMILES: *COC(=O)OCCCCCCCCCCCC] |
| —C₁₇H₃₅ | 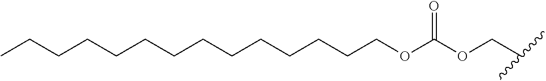 [SMILES: *COC(=O)OCCCCCCCCCCCCCC] |
| —C₁₇H₃₅ | 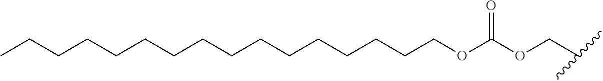 [SMILES: *COC(=O)OCCCCCCCCCCCCCCCC] |
| 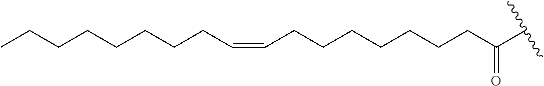 [SMILES: *C(=O)CCCCCCC/C=C\CCCCCCCC] | 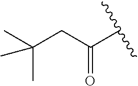 [SMILES: *C(=O)CC(C)(C)C] |
| 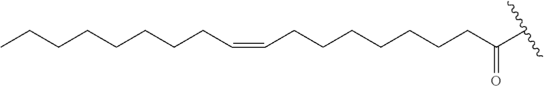 [SMILES: *C(=O)CCCCCCC/C=C\CCCCCCCC] | 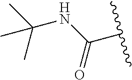 [SMILES: *C(=O)NC(C)(C)C] |
| 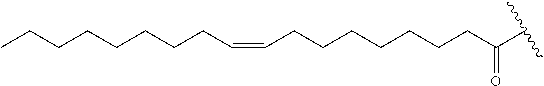 [SMILES: *C(=O)CCCCCCC/C=C\CCCCCCCC] | 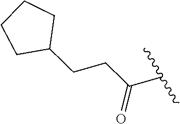 [SMILES: *C(=O)CCC1CCCC1] |
| 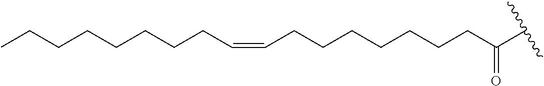 [SMILES: *C(=O)CCCCCCC/C=C\CCCCCCCC] | 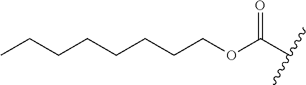 [SMILES: *C(=O)OCCCCCCCC] |
| 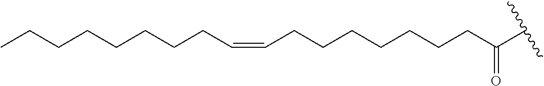 [SMILES: *C(=O)CCCCCCC/C=C\CCCCCCCC] | 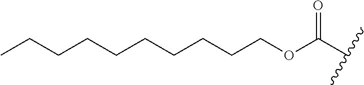 [SMILES: *C(=O)OCCCCCCCCCC] |
| 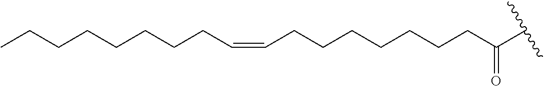 [SMILES: *C(=O)CCCCCCC/C=C\CCCCCCCC] | 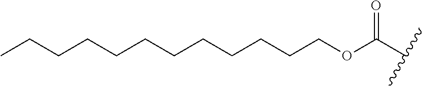 [SMILES: *C(=O)OCCCCCCCCCCCC] |
| 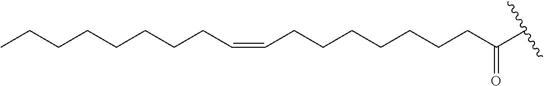 [SMILES: *C(=O)CCCCCCC/C=C\CCCCCCCC] | 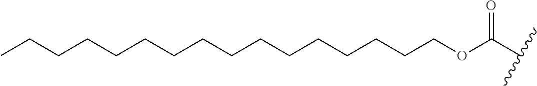 [SMILES: *C(=O)OCCCCCCCCCCCCCCCC] |
| 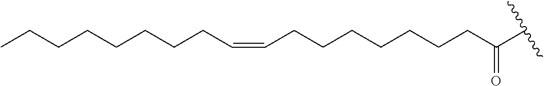 [SMILES: *C(=O)CCCCCCC/C=C\CCCCCCCC] | 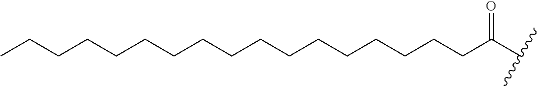 [SMILES: *C(=O)CCCCCCCCCCCCCCCCC] |
| 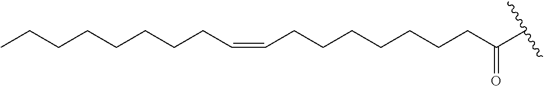 [SMILES: *C(=O)CCCCCCC/C=C\CCCCCCCC] | 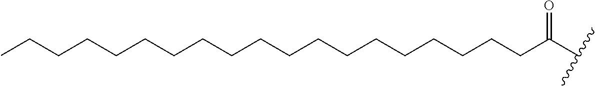 [SMILES: *C(=O)CCCCCCCCCCCCCCCCCCC] |
| 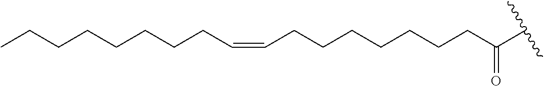 [SMILES: *C(=O)CCCCCCC/C=C\CCCCCCCC] | 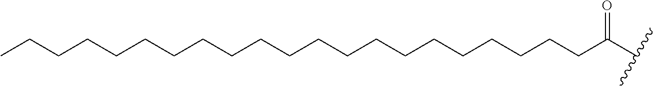 [SMILES: *C(=O)CCCCCCCCCCCCCCCCCCCCC] |
| 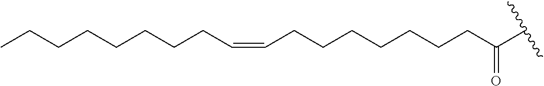 [SMILES: *C(=O)CCCCCCC/C=C\CCCCCCCC] | 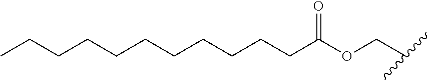 [SMILES: *COC(=O)CCCCCCCCCCC] |
| 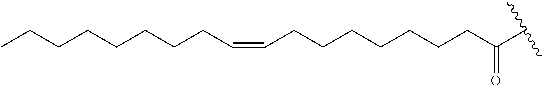 [SMILES: *C(=O)CCCCCCC/C=C\CCCCCCCC] | 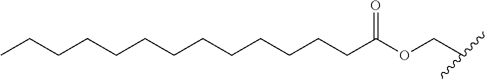 [SMILES: *COC(=O)CCCCCCCCCCCCC] |
| 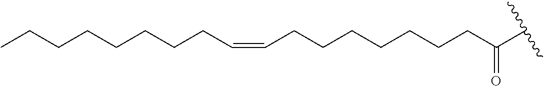 [SMILES: *C(=O)CCCCCCC/C=C\CCCCCCCC] | 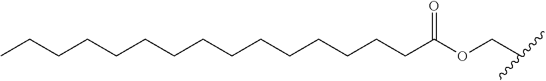 [SMILES: *COC(=O)CCCCCCCCCCCCCCC] |
| 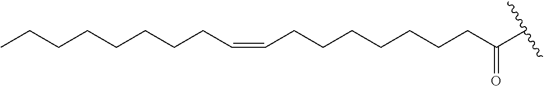 [SMILES: *C(=O)CCCCCCC/C=C\CCCCCCCC] | 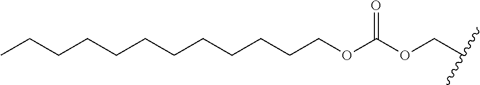 [SMILES: *COC(=O)OCCCCCCCCCCCC] |
| 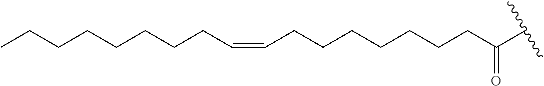 [SMILES: *C(=O)CCCCCCC/C=C\CCCCCCCC] | 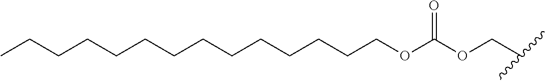 [SMILES: *COC(=O)OCCCCCCCCCCCCCC] |
| 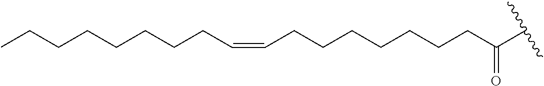 [SMILES: *C(=O)CCCCCCC/C=C\CCCCCCCC] | 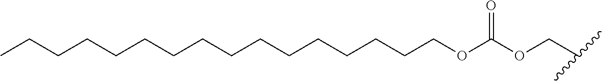 [SMILES: *COC(=O)OCCCCCCCCCCCCCCCC] |
| 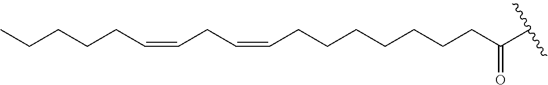 [SMILES: *C(=O)CCCCCCC/C=C\C/C=C\CCCCC] | 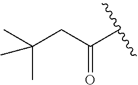 [SMILES: *C(=O)CC(C)(C)C] |
| 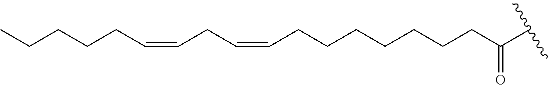 [SMILES: *C(=O)CCCCCCC/C=C\C/C=C\CCCCC] | 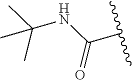 [SMILES: *C(=O)NC(C)(C)C] |
| 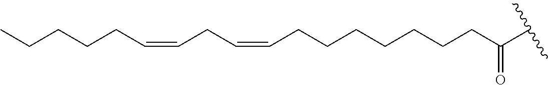 [SMILES: *C(=O)CCCCCCC/C=C\C/C=C\CCCCC] | 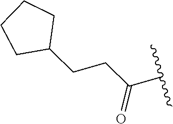 [SMILES: *C(=O)CCC1CCCC1] |
| 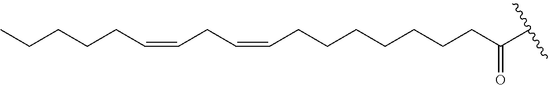 [SMILES: *C(=O)CCCCCCC/C=C\C/C=C\CCCCC] | 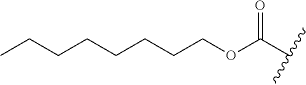 [SMILES: *C(=O)OCCCCCCCC] |
| 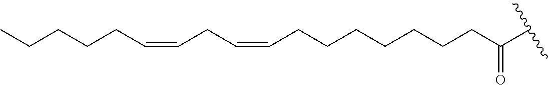 [SMILES: *C(=O)CCCCCCC/C=C\C/C=C\CCCCC] | 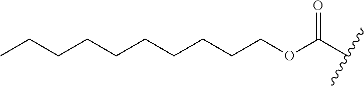 [SMILES: *C(=O)OCCCCCCCCCC] |
| 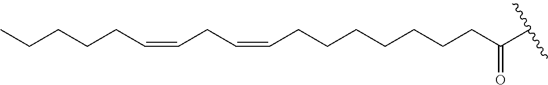 [SMILES: *C(=O)CCCCCCC/C=C\C/C=C\CCCCC] | 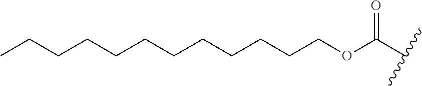 [SMILES: *C(=O)OCCCCCCCCCCCC] |
| 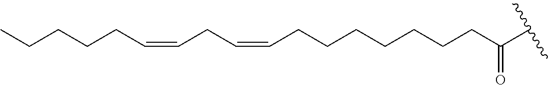 [SMILES: *C(=O)CCCCCCC/C=C\C/C=C\CCCCC] | 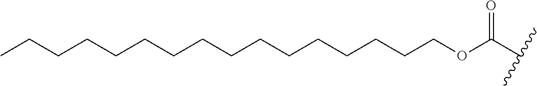 [SMILES: *C(=O)OCCCCCCCCCCCCCCCC] |
| 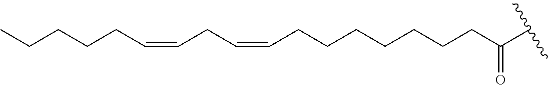 [SMILES: *C(=O)CCCCCCC/C=C\C/C=C\CCCCC] | 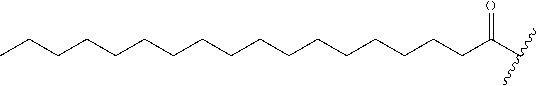 [SMILES: *C(=O)CCCCCCCCCCCCCCCCC] |
| 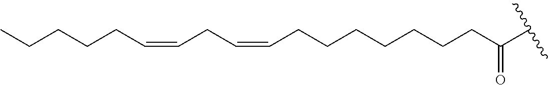 [SMILES: *C(=O)CCCCCCC/C=C\C/C=C\CCCCC] | 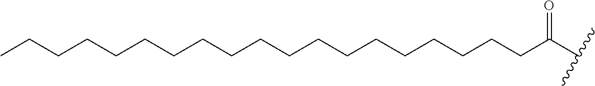 [SMILES: *C(=O)CCCCCCCCCCCCCCCCCCC] |
| 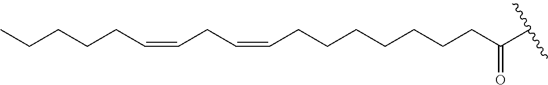 [SMILES: *C(=O)CCCCCCC/C=C\C/C=C\CCCCC] | 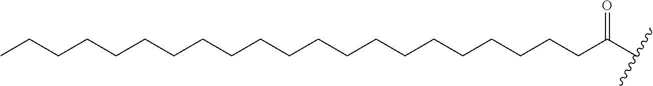 [SMILES: *C(=O)CCCCCCCCCCCCCCCCCCCCC] |
| 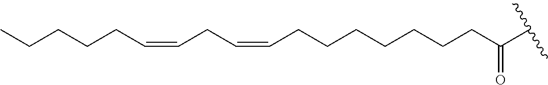 [SMILES: *C(=O)CCCCCCC/C=C\C/C=C\CCCCC] | 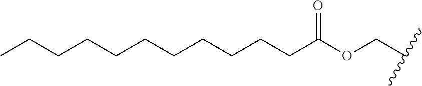 [SMILES: *COC(=O)CCCCCCCCCCC] |
| 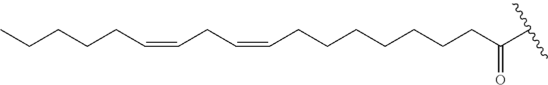 [SMILES: *C(=O)CCCCCCC/C=C\C/C=C\CCCCC] | 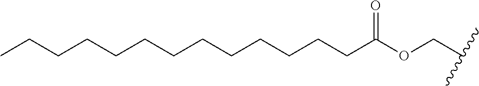 [SMILES: *COC(=O)CCCCCCCCCCCCC] |
| 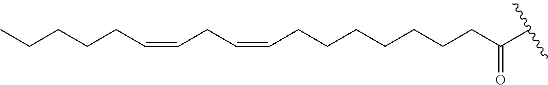 [SMILES: *C(=O)CCCCCCC/C=C\C/C=C\CCCCC] | 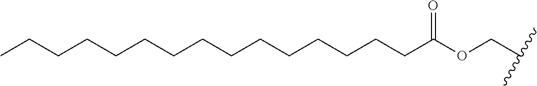 [SMILES: *COC(=O)CCCCCCCCCCCCCCC] |
| 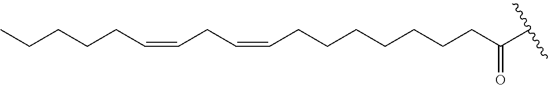 [SMILES: *C(=O)CCCCCCC/C=C\C/C=C\CCCCC] | 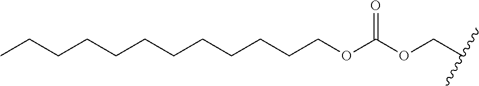 [SMILES: *COC(=O)OCCCCCCCCCCCC] |
| 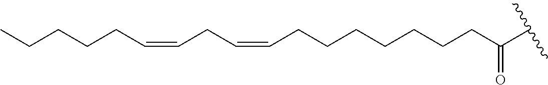 [SMILES: *C(=O)CCCCCCC/C=C\C/C=C\CCCCC] | 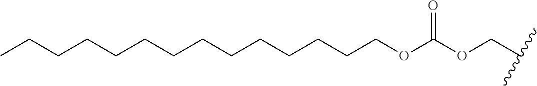 [SMILES: *COC(=O)OCCCCCCCCCCCCCC] |
| 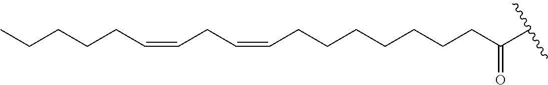 [SMILES: *C(=O)CCCCCCC/C=C\C/C=C\CCCCC] | 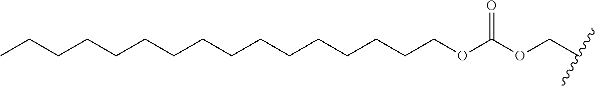 [SMILES: *COC(=O)OCCCCCCCCCCCCCCCC] |
| 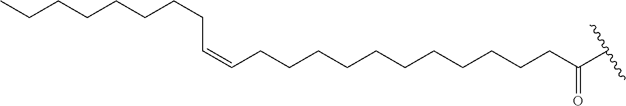 [SMILES: *C(=O)CCCCCCCCCCC/C=C\CCCCCCCC] | 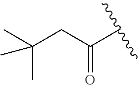 [SMILES: *C(=O)CC(C)(C)C] |
| 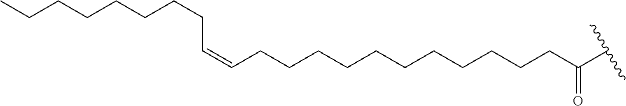 [SMILES: *C(=O)CCCCCCCCCCC/C=C\CCCCCCCC] | 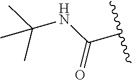 [SMILES: *C(=O)NC(C)(C)C] |
| 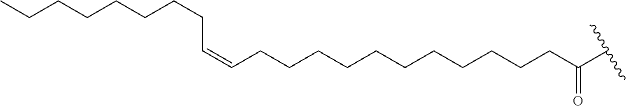 [SMILES: *C(=O)CCCCCCCCCCC/C=C\CCCCCCCC] | 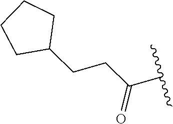 [SMILES: *C(=O)CCC1CCCC1] |
| 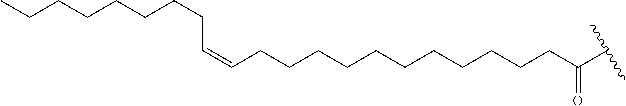 [SMILES: *C(=O)CCCCCCCCCCC/C=C\CCCCCCCC] | 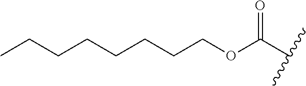 [SMILES: *C(=O)OCCCCCCCC] |
| 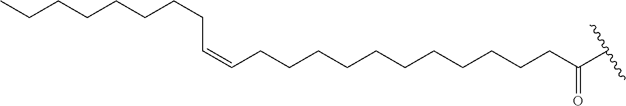 [SMILES: *C(=O)CCCCCCCCCCC/C=C\CCCCCCCC] | 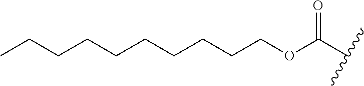 [SMILES: *C(=O)OCCCCCCCCCC] |
| 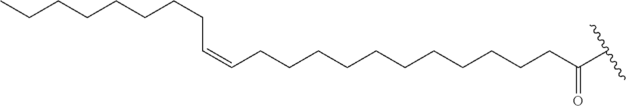 [SMILES: *C(=O)CCCCCCCCCCC/C=C\CCCCCCCC] | 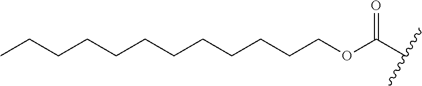 [SMILES: *C(=O)OCCCCCCCCCCCC] |
| 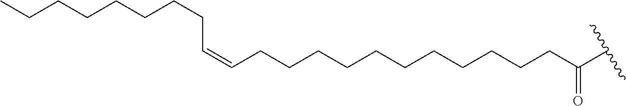 [SMILES: *C(=O)CCCCCCCCCCC/C=C\CCCCCCCC] | 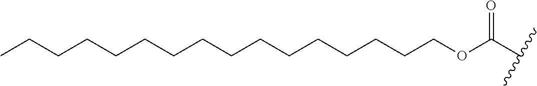 [SMILES: *C(=O)OCCCCCCCCCCCCCCCC] |
| 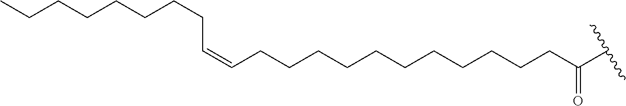 [SMILES: *C(=O)CCCCCCCCCCC/C=C\CCCCCCCC] | 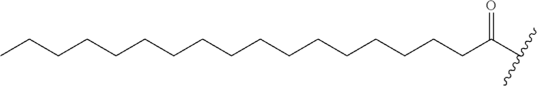 [SMILES: *C(=O)CCCCCCCCCCCCCCCCC] |
| 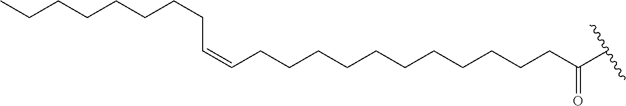 [SMILES: *C(=O)CCCCCCCCCCC/C=C\CCCCCCCC] | 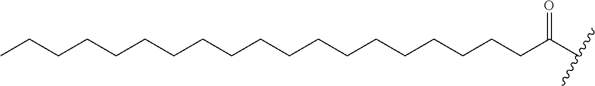 [SMILES: *C(=O)CCCCCCCCCCCCCCCCCCC] |
| 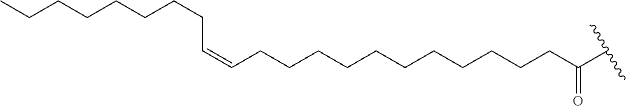 [SMILES: *C(=O)CCCCCCCCCCC/C=C\CCCCCCCC] | 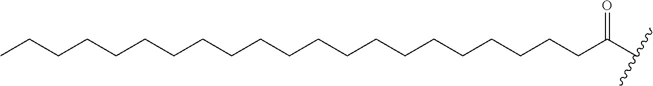 [SMILES: *C(=O)CCCCCCCCCCCCCCCCCCCCC] |
| 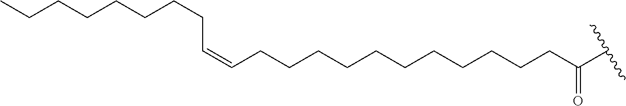 [SMILES: *C(=O)CCCCCCCCCCC/C=C\CCCCCCCC] | 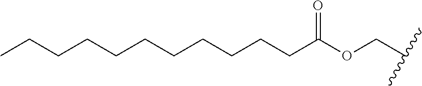 [SMILES: *COC(=O)CCCCCCCCCCC] |
| 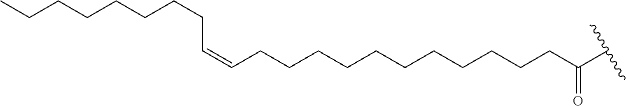 [SMILES: *C(=O)CCCCCCCCCCC/C=C\CCCCCCCC] | 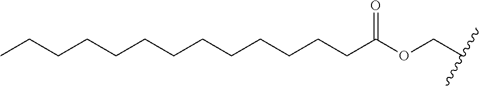 [SMILES: *COC(=O)CCCCCCCCCCCCC] |
| 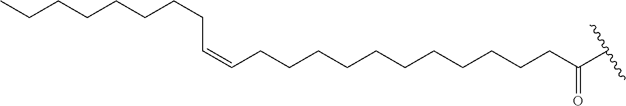 [SMILES: *C(=O)CCCCCCCCCCC/C=C\CCCCCCCC] | 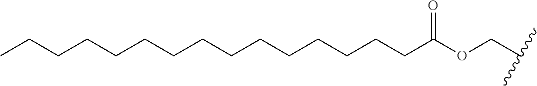 [SMILES: *COC(=O)CCCCCCCCCCCCCCC] |
| 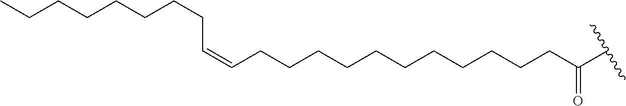 [SMILES: *C(=O)CCCCCCCCCCC/C=C\CCCCCCCC] | 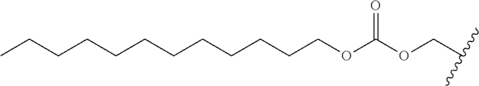 [SMILES: *COC(=O)OCCCCCCCCCCCC] |
| 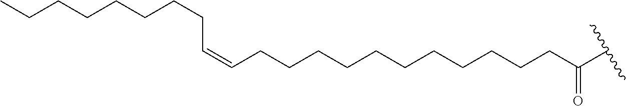 [SMILES: *C(=O)CCCCCCCCCCC/C=C\CCCCCCCC] | 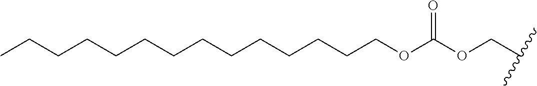 [SMILES: *COC(=O)OCCCCCCCCCCCCCC] |
| 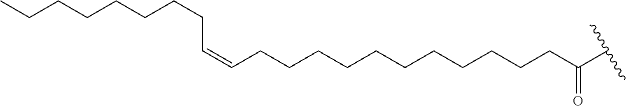 [SMILES: *C(=O)CCCCCCCCCCC/C=C\CCCCCCCC] | 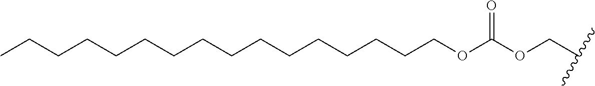 [SMILES: *COC(=O)OCCCCCCCCCCCCCCCC] |
| 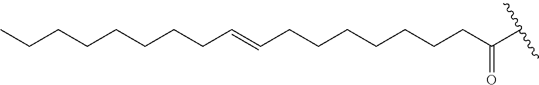 [SMILES: *C(=O)CCCCCCC/C=C/CCCCCCCC] | 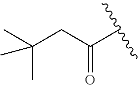 [SMILES: *C(=O)CC(C)(C)C] |
| 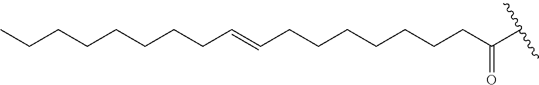 [SMILES: *C(=O)CCCCCCC/C=C/CCCCCCCC] | 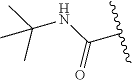 [SMILES: *C(=O)NC(C)(C)C] |
| 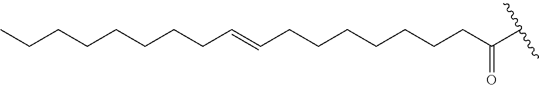 [SMILES: *C(=O)CCCCCCC/C=C/CCCCCCCC] | 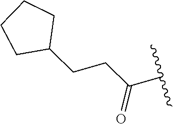 [SMILES: *C(=O)CCC1CCCC1] |
| 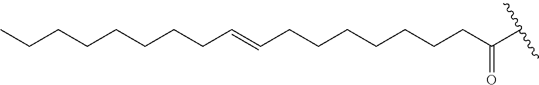 [SMILES: *C(=O)CCCCCCC/C=C/CCCCCCCC] | 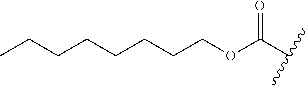 [SMILES: *C(=O)OCCCCCCCC] |
| 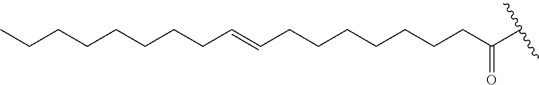 [SMILES: *C(=O)CCCCCCC/C=C/CCCCCCCC] | 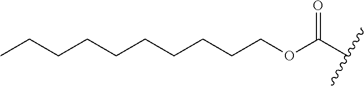 [SMILES: *C(=O)OCCCCCCCCCC] |
| 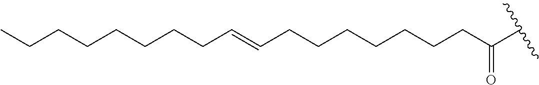 [SMILES: *C(=O)CCCCCCC/C=C/CCCCCCCC] | 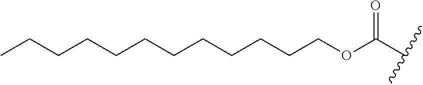 [SMILES: *C(=O)OCCCCCCCCCCCC] |
| 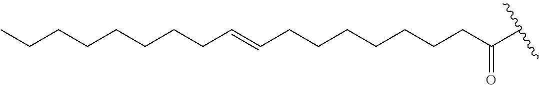 [SMILES: *C(=O)CCCCCCC/C=C/CCCCCCCC] | 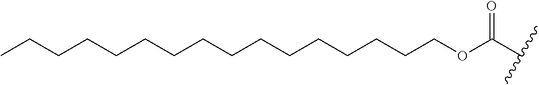 [SMILES: *C(=O)OCCCCCCCCCCCCCCCC] |
| 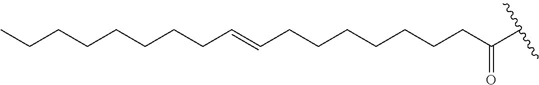 [SMILES: *C(=O)CCCCCCC/C=C/CCCCCCCC] | 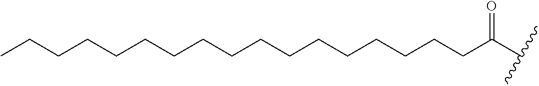 [SMILES: *C(=O)CCCCCCCCCCCCCCCCC] |
| 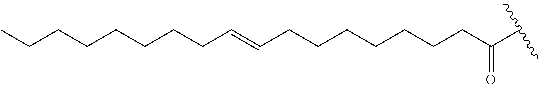 [SMILES: *C(=O)CCCCCCC/C=C/CCCCCCCC] | 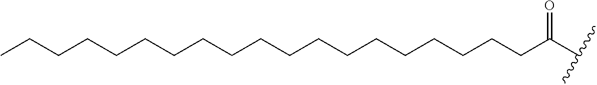 [SMILES: *C(=O)CCCCCCCCCCCCCCCCCCC] |
| 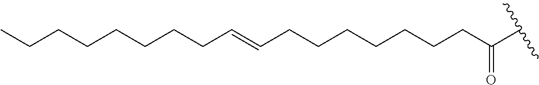 [SMILES: *C(=O)CCCCCCC/C=C/CCCCCCCC] | 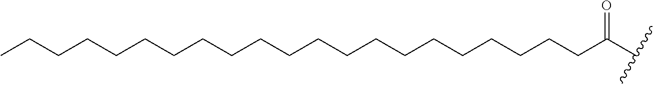 [SMILES: *C(=O)CCCCCCCCCCCCCCCCCCCCC] |
| 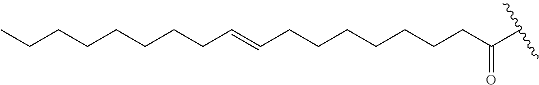 [SMILES: *C(=O)CCCCCCC/C=C/CCCCCCCC] | 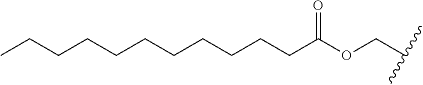 [SMILES: *COC(=O)CCCCCCCCCCC] |
| 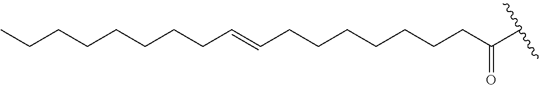 [SMILES: *C(=O)CCCCCCC/C=C/CCCCCCCC] | 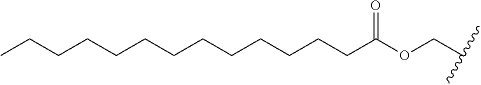 [SMILES: *COC(=O)CCCCCCCCCCCCC] |
| 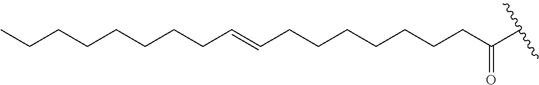 [SMILES: *C(=O)CCCCCCC/C=C/CCCCCCCC] | 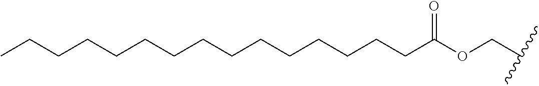 [SMILES: *COC(=O)CCCCCCCCCCCCCCC] |
| 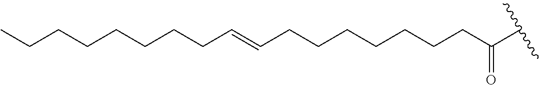 [SMILES: *C(=O)CCCCCCC/C=C/CCCCCCCC] | 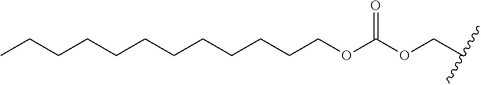 [SMILES: *COC(=O)OCCCCCCCCCCCC] |
| 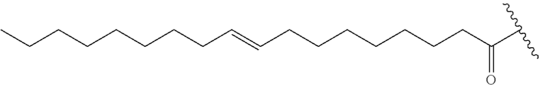 [SMILES: *C(=O)CCCCCCC/C=C/CCCCCCCC] | 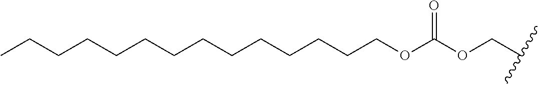 [SMILES: *COC(=O)OCCCCCCCCCCCCCC] |
| 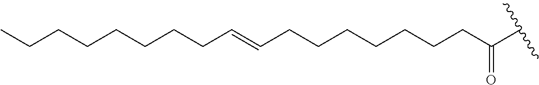 [SMILES: *C(=O)CCCCCCC/C=C/CCCCCCCC] | 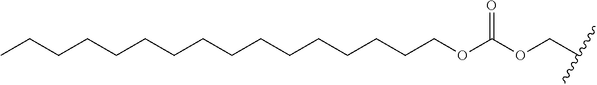 [SMILES: *COC(=O)OCCCCCCCCCCCCCCCC] |
| 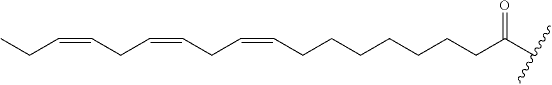 [SMILES: *C(=O)CCCCCCC/C=C\C/C=C\C/C=C\CC] | 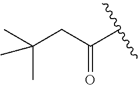 [SMILES: *C(=O)CC(C)(C)C] |
| 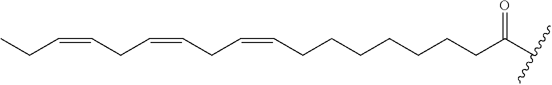 [SMILES: *C(=O)CCCCCCC/C=C\C/C=C\C/C=C\CC] | 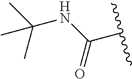 [SMILES: *C(=O)NC(C)(C)C] |
| 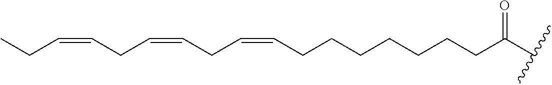 [SMILES: *C(=O)CCCCCCC/C=C\C/C=C\C/C=C\CC] | 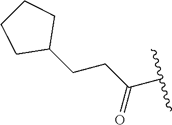 [SMILES: *C(=O)CCC1CCCC1] |
| 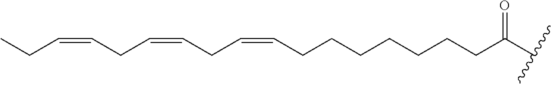 [SMILES: *C(=O)CCCCCCC/C=C\C/C=C\C/C=C\CC] | 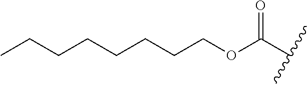 [SMILES: *C(=O)OCCCCCCCC] |
| 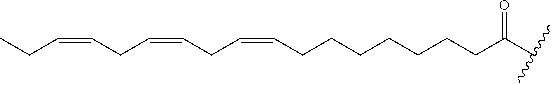 [SMILES: *C(=O)CCCCCCC/C=C\C/C=C\C/C=C\CC] | 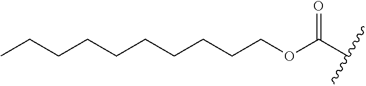 [SMILES: *C(=O)OCCCCCCCCCC] |
| 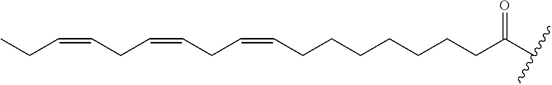 [SMILES: *C(=O)CCCCCCC/C=C\C/C=C\C/C=C\CC] | 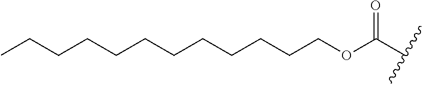 [SMILES: *C(=O)OCCCCCCCCCCCC] |
| 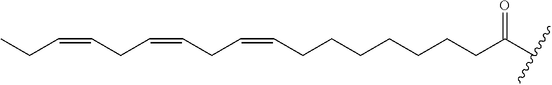 [SMILES: *C(=O)CCCCCCC/C=C\C/C=C\C/C=C\CC] | 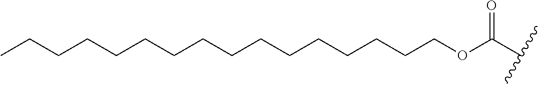 [SMILES: *C(=O)OCCCCCCCCCCCCCCCC] |
| 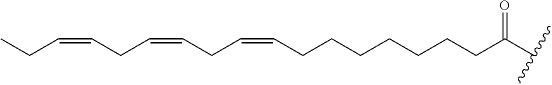 [SMILES: *C(=O)CCCCCCC/C=C\C/C=C\C/C=C\CC] | 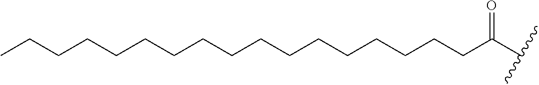 [SMILES: *C(=O)CCCCCCCCCCCCCCCCC] |
| 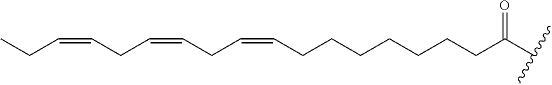 [SMILES: *C(=O)CCCCCCC/C=C\C/C=C\C/C=C\CC] | 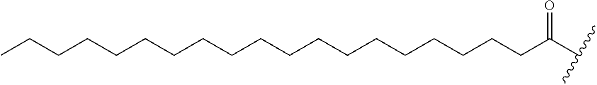 [SMILES: *C(=O)CCCCCCCCCCCCCCCCCCC] |
| 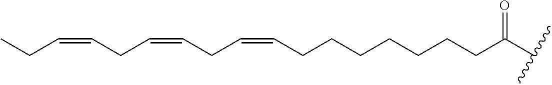 [SMILES: *C(=O)CCCCCCC/C=C\C/C=C\C/C=C\CC] | 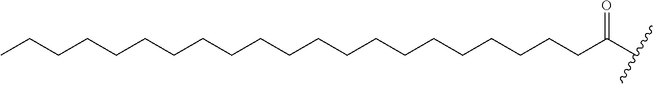 [SMILES: *C(=O)CCCCCCCCCCCCCCCCCCCCC] |
| 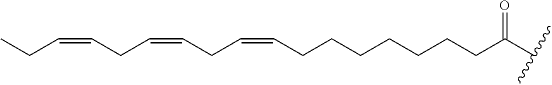 [SMILES: *C(=O)CCCCCCC/C=C\C/C=C\C/C=C\CC] | 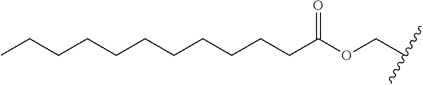 [SMILES: *COC(=O)CCCCCCCCCCC] |
| 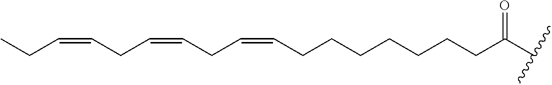 [SMILES: *C(=O)CCCCCCC/C=C\C/C=C\C/C=C\CC] | 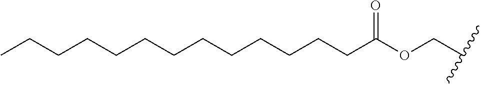 [SMILES: *COC(=O)CCCCCCCCCCCCC] |
| 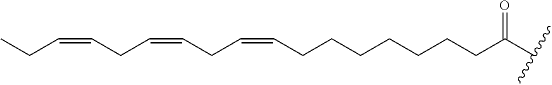 [SMILES: *C(=O)CCCCCCC/C=C\C/C=C\C/C=C\CC] | 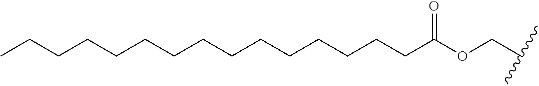 [SMILES: *COC(=O)CCCCCCCCCCCCCCC] |
| 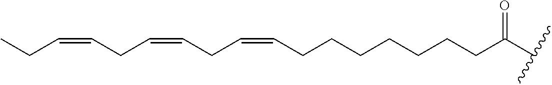 [SMILES: *C(=O)CCCCCCC/C=C\C/C=C\C/C=C\CC] | 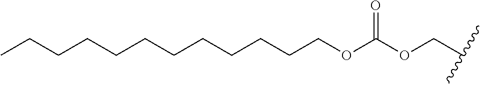 [SMILES: *COC(=O)OCCCCCCCCCCCC] |
| 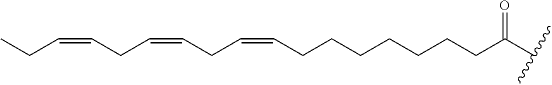 [SMILES: *C(=O)CCCCCCC/C=C\C/C=C\C/C=C\CC] | 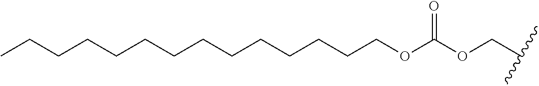 [SMILES: *COC(=O)OCCCCCCCCCCCCCC] |
| 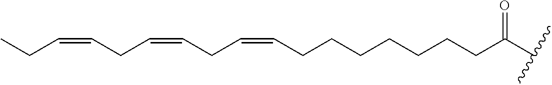 [SMILES: *C(=O)CCCCCCC/C=C\C/C=C\C/C=C\CC] | 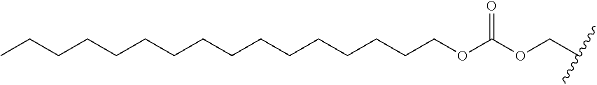 [SMILES: *COC(=O)OCCCCCCCCCCCCCCCC] |
| tBu | 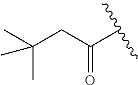 [SMILES: *C(=O)CC(C)(C)C] |
| tBu | 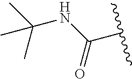 [SMILES: *C(=O)NC(C)(C)C] |
| tBu | 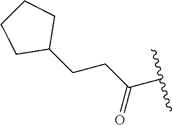 [SMILES: *C(=O)CCC1CCCC1] |
| tBu | 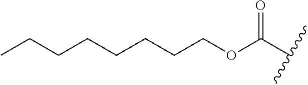 [SMILES: *C(=O)OCCCCCCCC] |
| tBu | 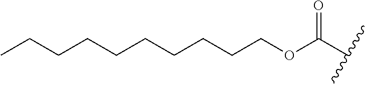 [SMILES: *C(=O)OCCCCCCCCCC] |
| tBu | 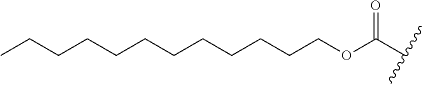 [SMILES: *C(=O)OCCCCCCCCCCCC] |
| tBu | 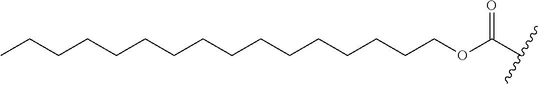 [SMILES: *C(=O)OCCCCCCCCCCCCCCCC] |
| tBu | 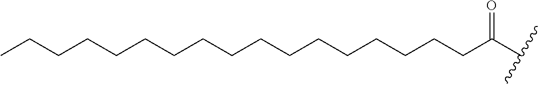 [SMILES: *C(=O)CCCCCCCCCCCCCCCCC] |
| tBu | 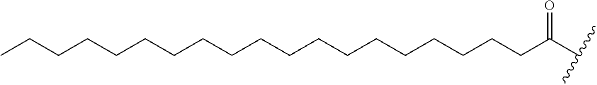 [SMILES: *C(=O)CCCCCCCCCCCCCCCCCCC] |
| tBu | 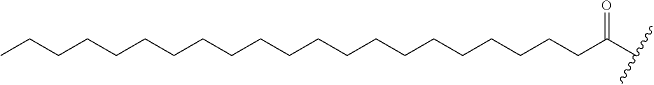 [SMILES: *C(=O)CCCCCCCCCCCCCCCCCCCCC] |
| tBu | 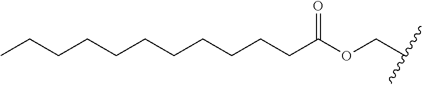 [SMILES: *COC(=O)CCCCCCCCCCC] |
| tBu | 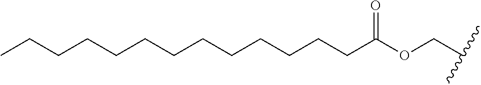 [SMILES: *COC(=O)CCCCCCCCCCCCC] |
| tBu | 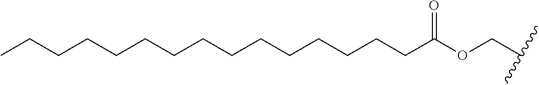 [SMILES: *COC(=O)CCCCCCCCCCCCCCC] |
| tBu | 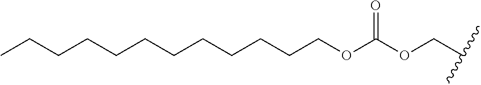 [SMILES: *COC(=O)OCCCCCCCCCCCC] |
| tBu | 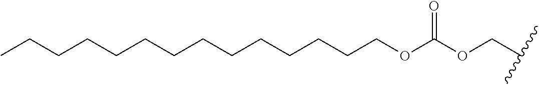 [SMILES: *COC(=O)OCCCCCCCCCCCCCC] |
| tBu | 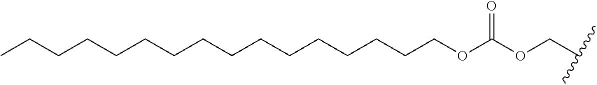 [SMILES: *COC(=O)OCCCCCCCCCCCCCCCC] |
| phenyl | 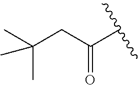 [SMILES: *C(=O)CC(C)(C)C] |
| phenyl | 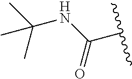 [SMILES: *C(=O)NC(C)(C)C] |
| phenyl | 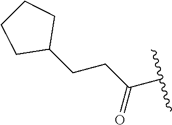 [SMILES: *C(=O)CCC1CCCC1] |
| phenyl | 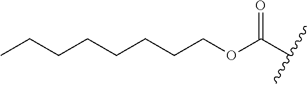 [SMILES: *C(=O)OCCCCCCCC] |
| phenyl | 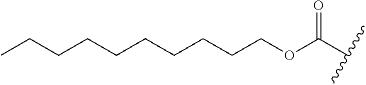 [SMILES: *C(=O)OCCCCCCCCCC] |
| phenyl | 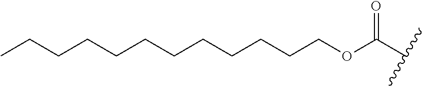 [SMILES: *C(=O)OCCCCCCCCCCCC] |
| phenyl | 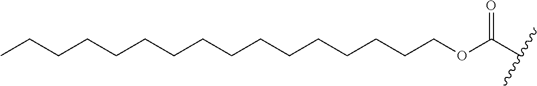 [SMILES: *C(=O)OCCCCCCCCCCCCCCCC] |
| phenyl | 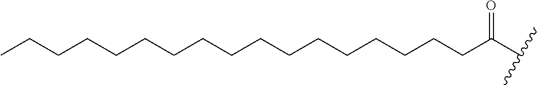 [SMILES: *C(=O)CCCCCCCCCCCCCCCCC] |
| phenyl | 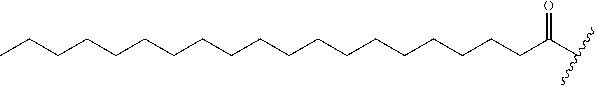 [SMILES: *C(=O)CCCCCCCCCCCCCCCCCCC] |
| phenyl | 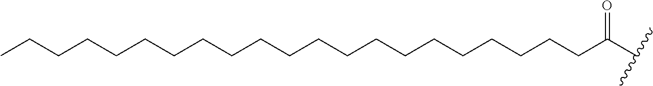 [SMILES: *C(=O)CCCCCCCCCCCCCCCCCCCCC] |
| phenyl | 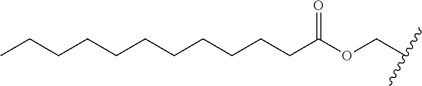 [SMILES: *COC(=O)CCCCCCCCCCC] |
| phenyl | 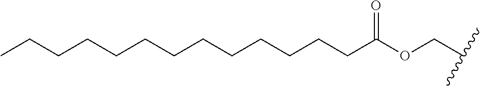 [SMILES: *COC(=O)CCCCCCCCCCCCC] |
| phenyl | 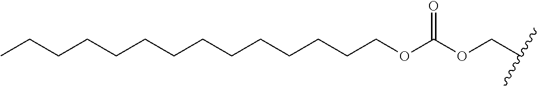 [SMILES: *COC(=O)OCCCCCCCCCCCCCC] |
| phenyl | 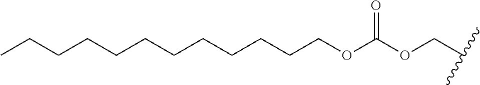 [SMILES: *COC(=O)OCCCCCCCCCCCC] |
| phenyl | 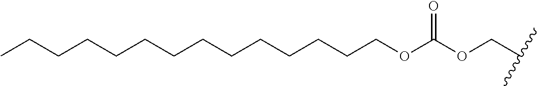 [SMILES: *COC(=O)OCCCCCCCCCCCCCC] |
| phenyl | 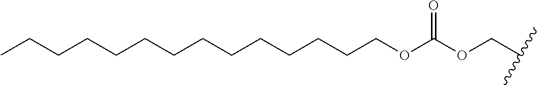 [SMILES: *COC(=O)OCCCCCCCCCCCCCC] |
| 4-chlorophenyl | 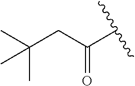 [SMILES: *C(=O)CC(C)(C)C] |
| 4-chlorophenyl | 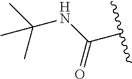 [SMILES: *C(=O)NC(C)(C)C] |
| 4-chlorophenyl | 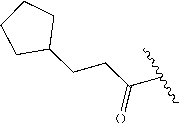 [SMILES: *C(=O)CCC1CCCC1] |
| 4-chlorophenyl | 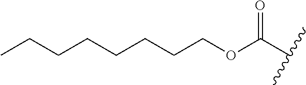 [SMILES: *C(=O)OCCCCCCCC] |
| 4-chlorophenyl | 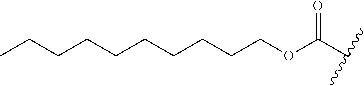 [SMILES: *C(=O)OCCCCCCCCCC] |
| 4-chlorophenyl | 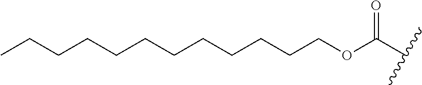 [SMILES: *C(=O)OCCCCCCCCCCCC] |
| 4-chlorophenyl | 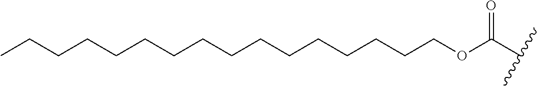 [SMILES: *C(=O)OCCCCCCCCCCCCCCCC] |
| 4-chlorophenyl | 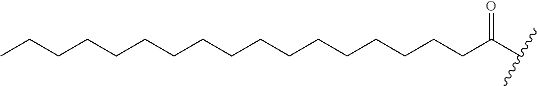 [SMILES: *C(=O)CCCCCCCCCCCCCCCCC] |
| 4-chlorophenyl | 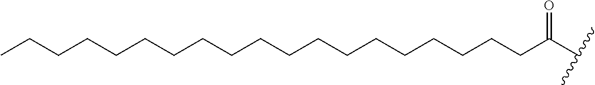 [SMILES: *C(=O)CCCCCCCCCCCCCCCCCCC] |
| 4-chlorophenyl | 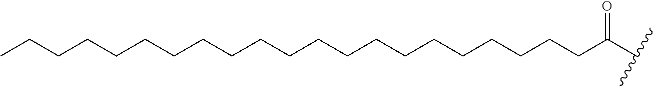 [SMILES: *C(=O)CCCCCCCCCCCCCCCCCCCCC] |
| 4-chlorophenyl | 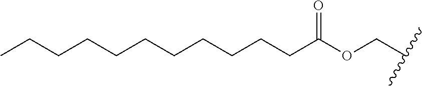 [SMILES: *COC(=O)CCCCCCCCCCC] |
| 4-chlorophenyl | 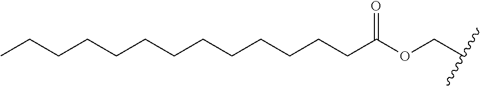 [SMILES: *COC(=O)CCCCCCCCCCCCC] |
| 4-chlorophenyl | 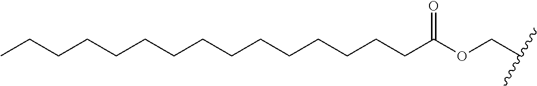 [SMILES: *COC(=O)CCCCCCCCCCCCCCC] |
| 4-chlorophenyl | 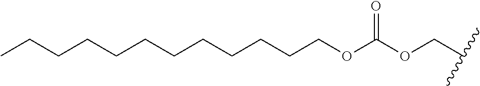 [SMILES: *COC(=O)OCCCCCCCCCCCC] |
| 4-chlorophenyl | 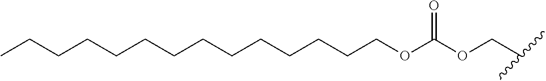 [SMILES: *COC(=O)OCCCCCCCCCCCCCC] |
| 4-chlorophenyl | 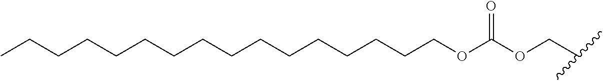 [SMILES: *COC(=O)OCCCCCCCCCCCCCCCC] |
| 3,4-dichlorophenyl | 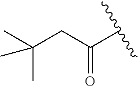 [SMILES: *C(=O)CC(C)(C)C] |
| 3,4-dichlorophenyl | 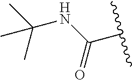 [SMILES: *C(=O)NC(C)(C)C] |
| 3,4-dichlorophenyl | 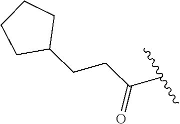 [SMILES: *C(=O)CCC1CCCC1] |
| 3,4-dichlorophenyl | 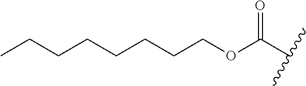 [SMILES: *C(=O)OCCCCCCCC] |
| 3,4-dichlorophenyl | 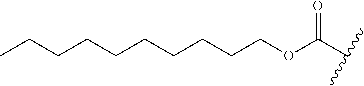 [SMILES: *C(=O)OCCCCCCCCCC] |
| 3,4-dichlorophenyl | 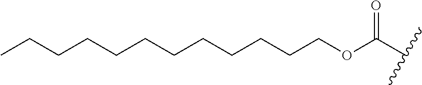 [SMILES: *C(=O)OCCCCCCCCCCCC] |
| 3,4-dichlorophenyl | 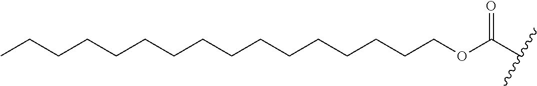 [SMILES: *C(=O)OCCCCCCCCCCCCCCCC] |
| 3,4-dichlorophenyl | 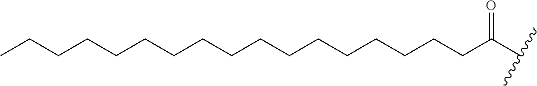 [SMILES: *C(=O)CCCCCCCCCCCCCCCCC] |
| 3,4-dichlorophenyl | 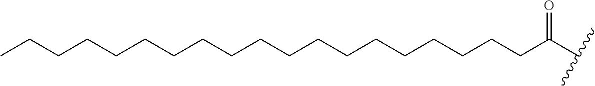 [SMILES: *C(=O)CCCCCCCCCCCCCCCCCCC] |
| 3,4-dichlorophenyl | 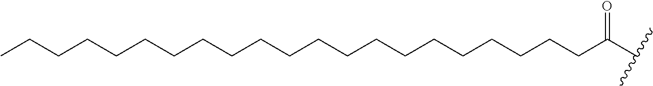 [SMILES: *C(=O)CCCCCCCCCCCCCCCCCCCCC] |
| 3,4-dichlorophenyl | 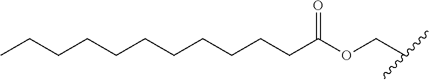 [SMILES: *COC(=O)CCCCCCCCCCC] |
| 3,4-dichlorophenyl | 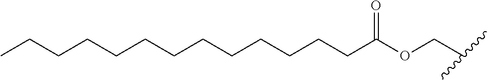 [SMILES: *COC(=O)CCCCCCCCCCCCC] |
| 3,4-dichlorophenyl | 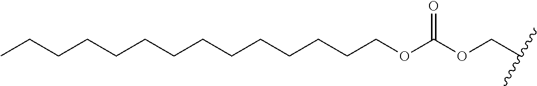 [SMILES: *COC(=O)OCCCCCCCCCCCCCC] |
| 3,4-dichlorophenyl | 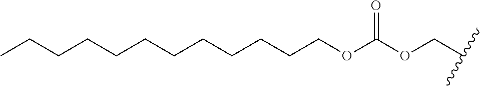 [SMILES: *COC(=O)OCCCCCCCCCCCC] |
| 3,4-dichlorophenyl | 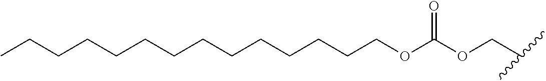 [SMILES: *COC(=O)OCCCCCCCCCCCCCC] |
| 3,4-dichlorophenyl | 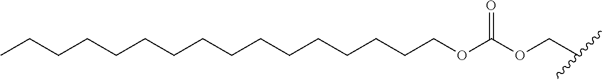 [SMILES: *COC(=O)OCCCCCCCCCCCCCCCC] |
| 4-methylphenyl | 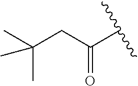 [SMILES: *C(=O)CC(C)(C)C] |
| 4-methylphenyl | 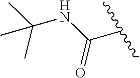 [SMILES: *C(=O)NC(C)(C)C] |
| 4-methylphenyl | 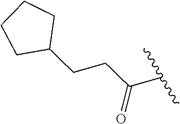 [SMILES: *C(=O)CCC1CCCC1] |
| 4-methylphenyl | 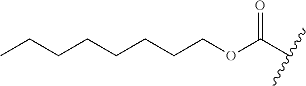 [SMILES: *C(=O)OCCCCCCCC] |
| 4-methylphenyl | 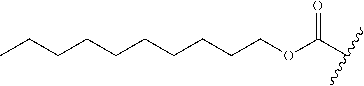 [SMILES: *C(=O)OCCCCCCCCCC] |
| 4-methylphenyl | 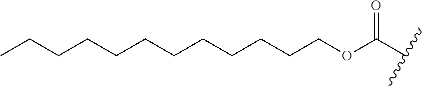 [SMILES: *C(=O)OCCCCCCCCCCCC] |
| 4-methylphenyl | 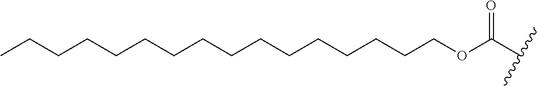 [SMILES: *C(=O)OCCCCCCCCCCCCCCCC] |
| 4-methylphenyl | 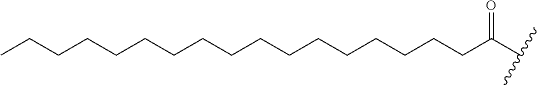 [SMILES: *C(=O)CCCCCCCCCCCCCCCCC] |
| 4-methylphenyl | 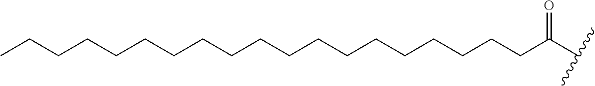 [SMILES: *C(=O)CCCCCCCCCCCCCCCCCCC] |
| 4-methylphenyl | 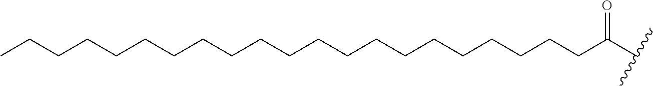 [SMILES: *C(=O)CCCCCCCCCCCCCCCCCCCCC] |
| 4-methylphenyl | 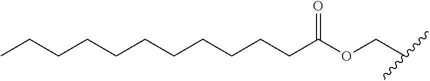 [SMILES: *COC(=O)CCCCCCCCCCC] |
| 4-methylphenyl | 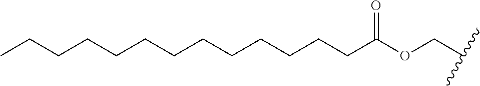 [SMILES: *COC(=O)CCCCCCCCCCCCC] |
| 4-methylphenyl | 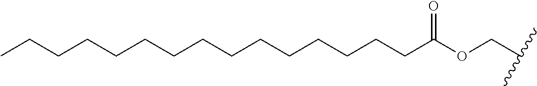 [SMILES: *COC(=O)CCCCCCCCCCCCCCC] |
| 4-methylphenyl | 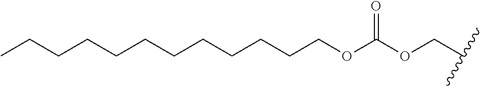 [SMILES: *COC(=O)OCCCCCCCCCCCC] |
| 4-methylphenyl | 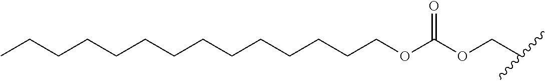 [SMILES: *COC(=O)OCCCCCCCCCCCCCC] |
| 4-methylphenyl | 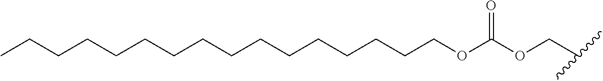 [SMILES: *COC(=O)OCCCCCCCCCCCCCCCC] |
| 4-methoxyphenyl | 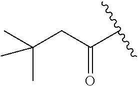 [SMILES: *C(=O)CC(C)(C)C] |
| 4-methoxyphenyl | 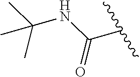 [SMILES: *C(=O)NC(C)(C)C] |
| 4-methoxyphenyl | 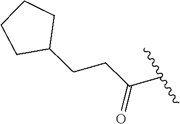 [SMILES: *C(=O)CCC1CCCC1] |
| 4-methoxyphenyl | 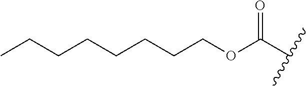 [SMILES: *C(=O)OCCCCCCCC] |
| 4-methoxyphenyl | 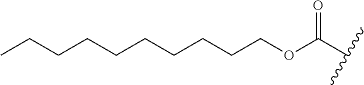 [SMILES: *C(=O)OCCCCCCCCCC] |
| 4-methoxyphenyl | 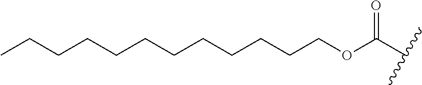 [SMILES: *C(=O)OCCCCCCCCCCCC] |
| 4-methoxyphenyl | 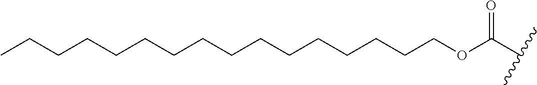 [SMILES: *C(=O)OCCCCCCCCCCCCCCCC] |
| 4-methoxyphenyl | 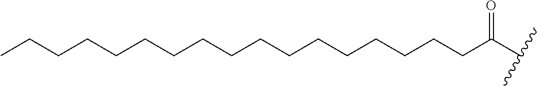 [SMILES: *C(=O)CCCCCCCCCCCCCCCCC] |
| 4-methoxyphenyl | 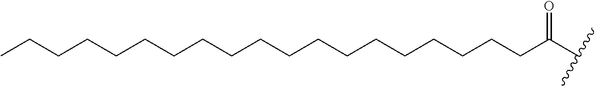 [SMILES: *C(=O)CCCCCCCCCCCCCCCCCCC] |
| 4-methoxyphenyl | 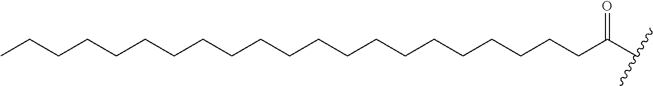 [SMILES: *C(=O)CCCCCCCCCCCCCCCCCCCCC] |
| 4-methoxyphenyl | 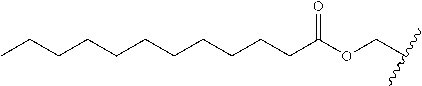 [SMILES: *COC(=O)CCCCCCCCCCC] |
| 4-methoxyphenyl | 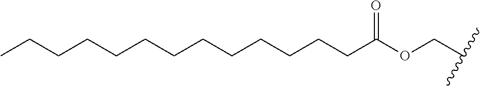 [SMILES: *COC(=O)CCCCCCCCCCCCC] |
| 4-methoxyphenyl | 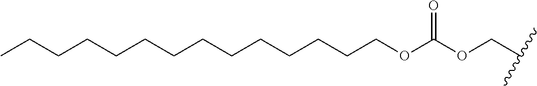 [SMILES: *COC(=O)OCCCCCCCCCCCCCC] |
| 4-methoxyphenyl | 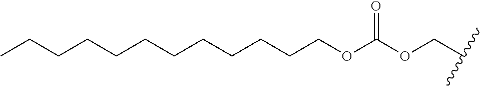 [SMILES: *COC(=O)OCCCCCCCCCCCC] |
| 4-methoxyphenyl | 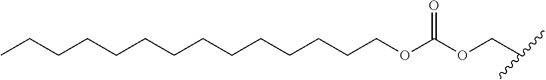 [SMILES: *COC(=O)OCCCCCCCCCCCCCC] |
| 4-methoxyphenyl | 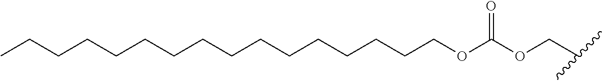 [SMILES: *COC(=O)OCCCCCCCCCCCCCCCC] |

In a 55^{th} aspect provided herein is a compound, or pharmaceutically acceptable salt thereof, having a structure provided in Formula (I-18),

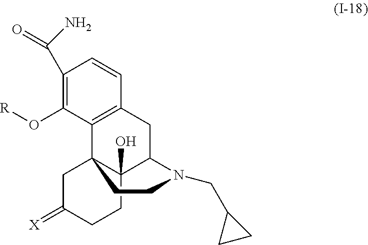

wherein X is ═O, R is R⁴NHC(O)—O—CH(R³)— and R³ and R⁴ are any of the pair of R³/R⁴ groups recited in Table 8. Alternatively, X is ═CH₂, R is R⁴NHC(O)—O—CH(R³)— and R³ and R⁴ are any of the pair of R³/R⁴ groups recited in Table 8.

In a 56^{th} aspect, provided herein is a compound, or pharmaceutically acceptable salt thereof, having a structure provided in Formula (II-18),

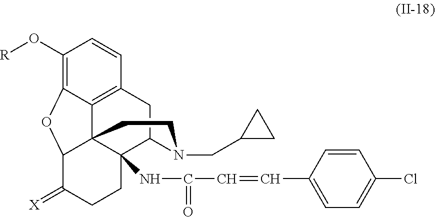

wherein X is ═O, R is R⁴NHC(O)—O—CH(R³)— and R³ and R⁴ are any of the pair of R³/R⁴ groups recited in Table 8. Alternatively, X is ═CH₂, R is R⁴NHC(O)—O—CH(R³)— and R³ and R⁴ are any of the pair of R³/R⁴ groups recited in Table 8.

In a 57^{th} aspect, provided herein is a compound, or pharmaceutically acceptable salt thereof, having a structure provided in Formula (III-18),

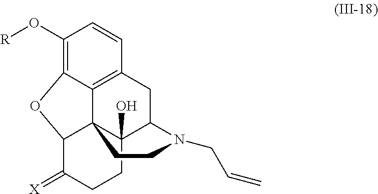

wherein X is ═O, R is R⁴HNC(O)—O—CH(R³)— and R³ and R⁴ are any of the pair of R³/R⁴ groups recited in Table 8. Alternatively, X is ═CH₂, R is R⁴NHC(O)—O—CH(R³)— and R³ and R⁴ are any of the pair of R³/R⁴ groups recited in Table 8.

TABLE 8

| R³ | R⁴ |
|---|---|
| H | Me |
| H | —C₇H₁₅ |
| H | —C₉H₁₉ |
| H | —C₁₁H₂₃ |
| H | —C₁₃H₂₇ |
| H | —C₁₅H₃₁ |
| H | —C₁₇H₃₅ |
| H | 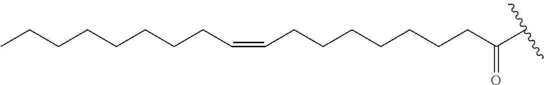 [SMILES: *C(=O)CCCCCCC/C=C\CCCCCCCC] |
| H | 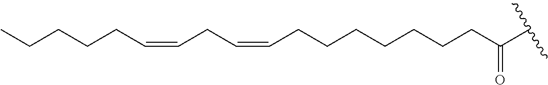 [SMILES: *C(=O)CCCCCCC/C=C\C/C=C\CCCCC] |
| H | 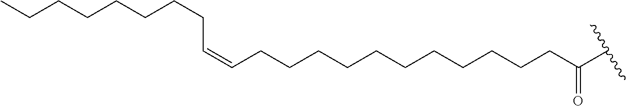 [SMILES: *C(=O)CCCCCCCCCCC/C=C\CCCCCCCC] |
| H | 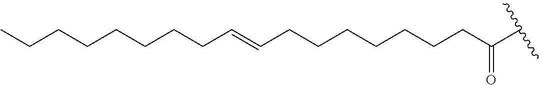 [SMILES: *C(=O)CCCCCCC/C=C/CCCCCCCC] |
| H | 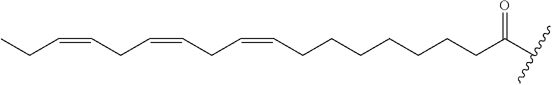 [SMILES: *C(=O)CCCCCCC/C=C\C/C=C\C/C=C\CC] |
| H | tBu |
| H | phenyl |
| H | 4-chlorophenyl |
| H | 3,4-dichlorophenyl |
| H | 4-methylphenyl |
| H | 4-methoxyphenyl |
| Me | Me |
| Me | —C₇H₁₅ |
| Me | —C₉H₁₉ |
| Me | —C₁₁H₂₃ |
| Me | —C₁₃H₂₇ |
| Me | —C₁₅H₃₁ |
| Me | —C₁₇H₃₅ |
| Me | 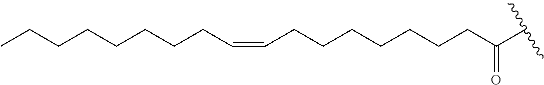 [SMILES: *C(=O)CCCCCCC/C=C\CCCCCCCC] |
| Me | 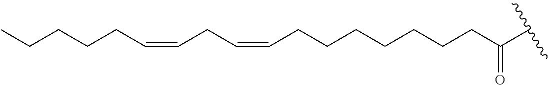 [SMILES: *C(=O)CCCCCCC/C=C\C/C=C\CCCCC] |
| Me | 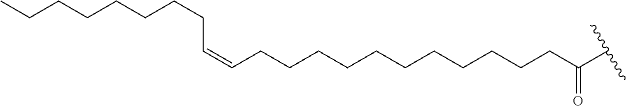 [SMILES: *C(=O)CCCCCCCCCCC/C=C\CCCCCCCC] |
| Me | 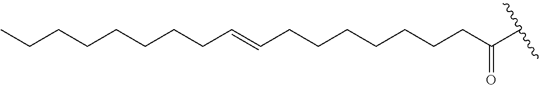 [SMILES: *C(=O)CCCCCCC/C=C/CCCCCCCC] |
| Me | 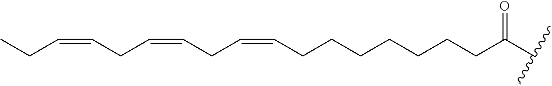 [SMILES: *C(=O)CCCCCCC/C=C\C/C=C\C/C=C\CC] |
| Me | tBu |
| Me | phenyl |
| Me | 4-chlorophenyl |
| Me | 3,4-dichlorophenyl |
| Me | 4-methylphenyl |
| Me | 4-methoxyphenyl |
| tBu | Me |
| tBu | —C₇H₁₅ |
| tBu | —C₉H₁₉ |
| tBu | —C₁₁H₂₃ |
| tBu | —C₁₃H₂₇ |
| tBu | —C₁₅H₃₁ |
| tBu | —C₁₇H₃₅ |
| tBu | 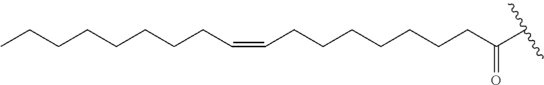 [SMILES: *C(=O)CCCCCCC/C=C\CCCCCCCC] |
| tBu | 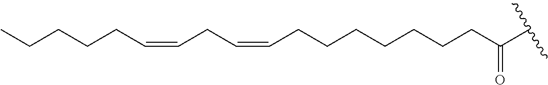 [SMILES: *C(=O)CCCCCCC/C=C\C/C=C\CCCCC] |
| tBu | 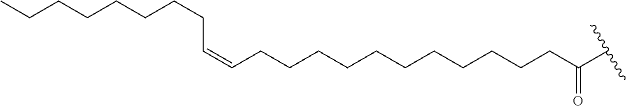 [SMILES: *C(=O)CCCCCCCCCCC/C=C\CCCCCCCC] |
| tBu | 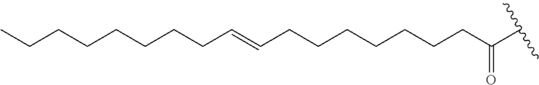 [SMILES: *C(=O)CCCCCCC/C=C/CCCCCCCC] |
| tBu | 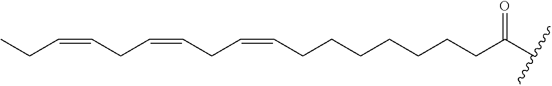 [SMILES: *C(=O)CCCCCCC/C=C\C/C=C\C/C=C\CC] |
| tBu | phenyl |
| tBu | tBu |
| tBu | 4-chlorophenyl |
| tBu | 3,4-dichlorophenyl |
| tBu | 4-methylphenyl |
| tBu | 4-methoxyphenyl |
| —C₇H₁₅ | Me |
| —C₇H₁₅ | —C₇H₁₅ |
| —C₇H₁₅ | —C₉H₁₉ |
| —C₇H₁₅ | —C₁₁H₂₃ |
| —C₇H₁₅ | —C₁₃H₂₇ |
| —C₇H₁₅ | —C₁₅H₃₁ |
| —C₇H₁₅ | —C₁₇H₃₅ |
| —C₇H₁₅ | 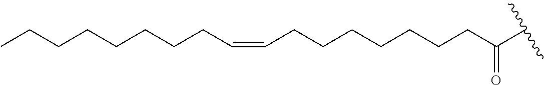 [SMILES: *C(=O)CCCCCCC/C=C\CCCCCCCC] |
| —C₇H₁₅ | 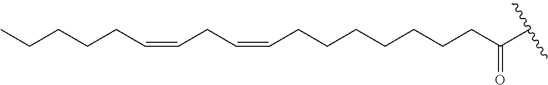 [SMILES: *C(=O)CCCCCCC/C=C\C/C=C\CCCCC] |
| —C₇H₁₅ | 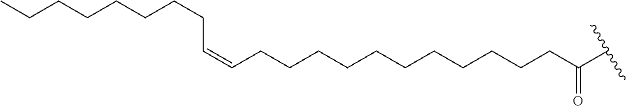 [SMILES: *C(=O)CCCCCCCCCCC/C=C\CCCCCCCC] |
| —C₇H₁₅ | 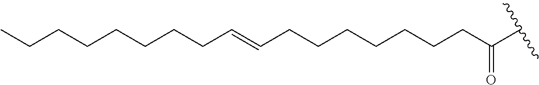 [SMILES: *C(=O)CCCCCCC/C=C/CCCCCCCC] |
| —C₇H₁₅ | 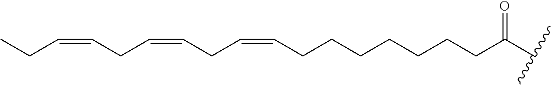 [SMILES: *C(=O)CCCCCCC/C=C\C/C=C\C/C=C\CC] |
| —C₇H₁₅ | tBu |
| —C₇H₁₅ | phenyl |
| —C₇H₁₅ | 4-chlorophenyl |
| —C₇H₁₅ | 3,4-dichlorophenyl |
| —C₇H₁₅ | 4-methylphenyl |
| —C₇H₁₅ | 4-methoxyphenyl |
| —C₉H₁₉ | Me |
| —C₉H₁₉ | —C₇H₁₅ |
| —C₉H₁₉ | —C₉H₁₉ |
| —C₉H₁₉ | —C₁₁H₂₃ |
| —C₉H₁₉ | —C₁₃H₂₇ |
| —C₉H₁₉ | —C₁₅H₃₁ |
| —C₉H₁₉ | —C₁₇H₃₅ |
| —C₉H₁₉ | 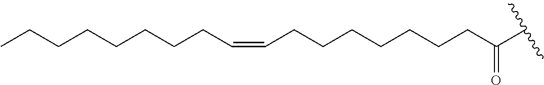 [SMILES: *C(=O)CCCCCCC/C=C\CCCCCCCC] |
| —C₉H₁₉ | 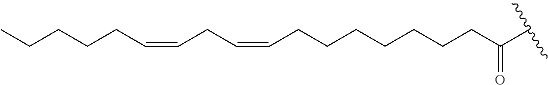 [SMILES: *C(=O)CCCCCCC/C=C\C/C=C\CCCCC] |
| —C₉H₁₉ | 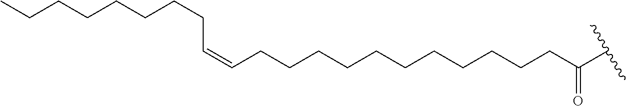 [SMILES: *C(=O)CCCCCCCCCCC/C=C\CCCCCCCC] |
| —C₉H₁₉ | 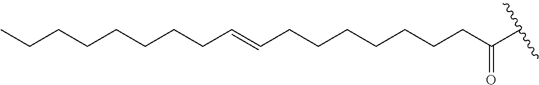 [SMILES: *C(=O)CCCCCCC/C=C/CCCCCCCC] |
| —C₉H₁₉ | 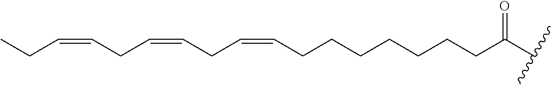 [SMILES: *C(=O)CCCCCCC/C=C\C/C=C\C/C=C\CC] |
| —C₉H₁₉ | tBu |
| —C₉H₁₉ | phenyl |
| —C₉H₁₉ | 4-chlorophenyl |
| —C₉H₁₉ | 3,4-dichlorophenyl |
| —C₉H₁₉ | 4-methylphenyl |
| —C₉H₁₉ | 4-methoxyphenyl |
| —C₁₁H₂₃ | Me |
| —C₁₁H₂₃ | —C₇H₁₅ |
| —C₁₁H₂₃ | —C₉H₁₉ |
| —C₁₁H₂₃ | —C₁₁H₂₃ |
| —C₁₁H₂₃ | —C₁₃H₂₇ |
| —C₁₁H₂₃ | —C₁₅H₃₁ |
| —C₁₁H₂₃ | —C₁₇H₃₅ |
| —C₁₁H₂₃ | 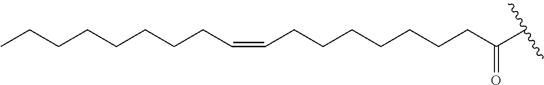 [SMILES: *C(=O)CCCCCCC/C=C\CCCCCCCC] |
| —C₁₁H₂₃ | 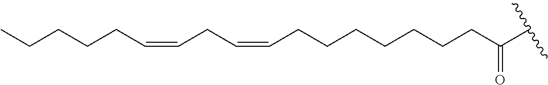 [SMILES: *C(=O)CCCCCCC/C=C\C/C=C\CCCCC] |
| —C₁₁H₂₃ | 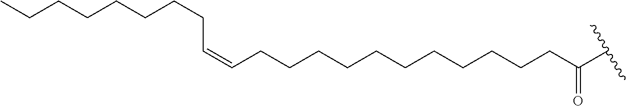 [SMILES: *C(=O)CCCCCCCCCCC/C=C\CCCCCCCC] |
| —C₁₁H₂₃ | 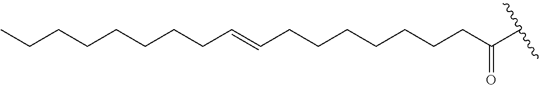 [SMILES: *C(=O)CCCCCCC/C=C/CCCCCCCC] |
| —C₁₁H₂₃ | 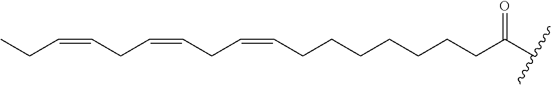 [SMILES: *C(=O)CCCCCCC/C=C\C/C=C\C/C=C\CC] |
| —C₁₁H₂₃ | tBu |
| —C₁₁H₂₃ | phenyl |
| —C₁₁H₂₃ | 4-chlorophenyl |
| —C₁₁H₂₃ | 3,4-dichlorophenyl |
| —C₁₁H₂₃ | 4-methylphenyl |
| —C₁₁H₂₃ | 4-methoxyphenyl |
| —C₁₃H₂₇ | Me |
| —C₁₃H₂₇ | —C₇H₁₅ |
| —C₁₃H₂₇ | —C₉H₁₉ |
| —C₁₃H₂₇ | —C₁₁H₂₃ |
| —C₁₃H₂₇ | —C₁₃H₂₇ |
| —C₁₃H₂₇ | —C₁₅H₃₁ |
| —C₁₃H₂₇ | —C₁₇H₃₅ |
| —C₁₃H₂₇ | 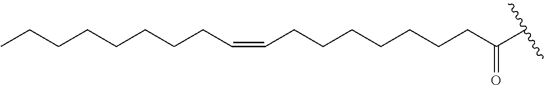 [SMILES: *C(=O)CCCCCCC/C=C\CCCCCCCC] |
| —C₁₃H₂₇ | 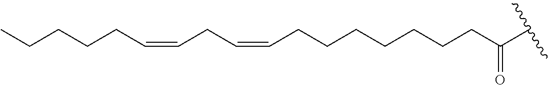 [SMILES: *C(=O)CCCCCCC/C=C\C/C=C\CCCCC] |
| —C₁₃H₂₇ | 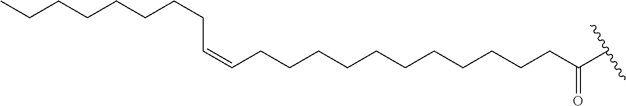 [SMILES: *C(=O)CCCCCCCCCCC/C=C\CCCCCCCC] |
| —C₁₃H₂₇ | 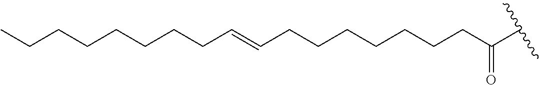 [SMILES: *C(=O)CCCCCCC/C=C/CCCCCCCC] |
| —C₁₃H₂₇ | 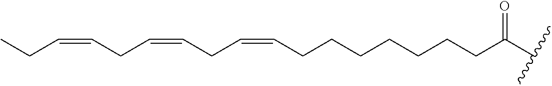 [SMILES: *C(=O)CCCCCCC/C=C\C/C=C\C/C=C\CC] |
| —C₁₃H₂₇ | tBu |
| —C₁₃H₂₇ | phenyl |
| —C₁₃H₂₇ | 4-chlorophenyl |
| —C₁₃H₂₇ | 3,4-dichlorophenyl |
| —C₁₃H₂₇ | 4-methylphenyl |
| —C₁₃H₂₇ | 4-methoxyphenyl |
| —C₁₅H₃₁ | Me |
| —C₁₅H₃₁ | —C₇H₁₅ |
| —C₁₅H₃₁ | —C₉H₁₉ |
| —C₁₅H₃₁ | —C₁₁H₂₃ |
| —C₁₅H₃₁ | —C₁₃H₂₇ |
| —C₁₅H₃₁ | —C₁₅H₃₁ |
| —C₁₅H₃₁ | —C₁₇H₃₅ |
| —C₁₅H₃₁ | 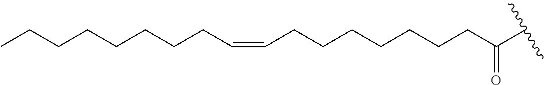 [SMILES: *C(=O)CCCCCCC/C=C\CCCCCCCC] |
| —C₁₅H₃₁ | 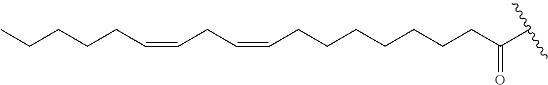 [SMILES: *C(=O)CCCCCCC/C=C\C/C=C\CCCCC] |
| —C₁₅H₃₁ | 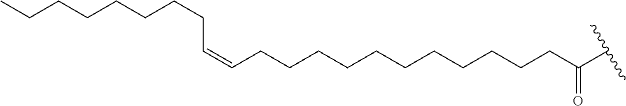 [SMILES: *C(=O)CCCCCCCCCCC/C=C\CCCCCCCC] |
| —C₁₅H₃₁ | 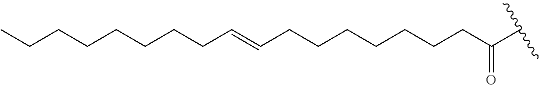 [SMILES: *C(=O)CCCCCCC/C=C/CCCCCCCC] |
| —C₁₅H₃₁ | 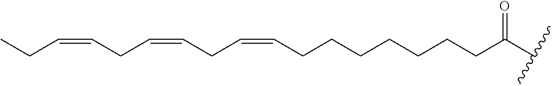 [SMILES: *C(=O)CCCCCCC/C=C\C/C=C\C/C=C\CC] |
| —C₁₅H₃₁ | tBu |
| —C₁₅H₃₁ | phenyl |
| —C₁₅H₃₁ | 4-chlorophenyl |
| —C₁₅H₃₁ | 3,4-dichlorophenyl |
| —C₁₅H₃₁ | 4-methylphenyl |
| —C₁₅H₃₁ | 4-methoxyphenyl |
| —C₁₇H₃₅ | Me |
| —C₁₇H₃₅ | —C₇H₁₅ |
| —C₁₇H₃₅ | —C₉H₁₉ |
| —C₁₇H₃₅ | —C₁₁H₂₃ |
| —C₁₇H₃₅ | —C₁₃H₂₇ |
| —C₁₇H₃₅ | —C₁₅H₃₁ |
| —C₁₇H₃₅ | —C₁₇H₃₅ |
| —C₁₇H₃₅ | 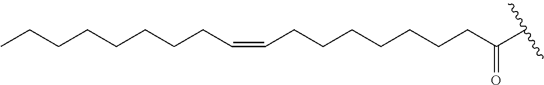 [SMILES: *C(=O)CCCCCCC/C=C\CCCCCCCC] |
| —C₁₇H₃₅ | 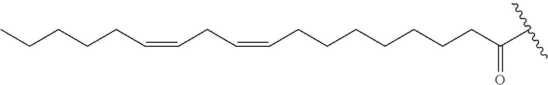 [SMILES: *C(=O)CCCCCCC/C=C\C/C=C\CCCCC] |
| —C₁₇H₃₅ | 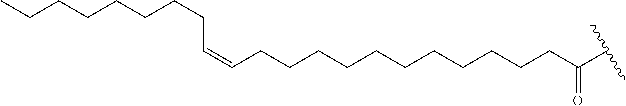 [SMILES: *C(=O)CCCCCCCCCCC/C=C\CCCCCCCC] |
| —C₁₇H₃₅ | 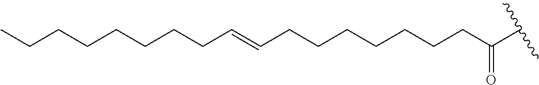 [SMILES: *C(=O)CCCCCCC/C=C/CCCCCCCC] |
| —C₁₇H₃₅ | 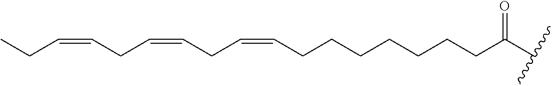 [SMILES: *C(=O)CCCCCCC/C=C\C/C=C\C/C=C\CC] |
| —C₁₇H₃₅ | tBu |
| —C₁₇H₃₅ | phenyl |
| —C₁₇H₃₅ | 4-chlorophenyl |
| —C₁₇H₃₅ | 3,4-dichlorophenyl |
| —C₁₇H₃₅ | 4-methylphenyl |
| —C₁₇H₃₅ | 4-methoxyphenyl |
| Phenyl | Me |
| Phenyl | —C₇H₁₅ |
| Phenyl | —C₉H₁₉ |
| Phenyl | —C₁₁H₂₃ |
| Phenyl | —C₁₃H₂₇ |
| Phenyl | —C₁₅H₃₁ |
| Phenyl | —C₁₇H₃₅ |
| Phenyl | 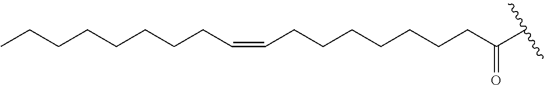 [SMILES: *C(=O)CCCCCCC/C=C\CCCCCCCC] |
| Phenyl | 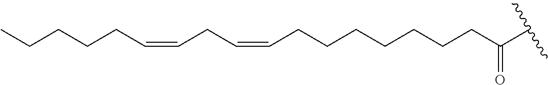 [SMILES: *C(=O)CCCCCCC/C=C\C/C=C\CCCCC] |
| Phenyl | 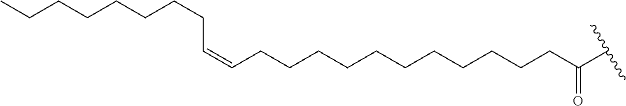 [SMILES: *C(=O)CCCCCCCCCCC/C=C\CCCCCCCC] |
| Phenyl | 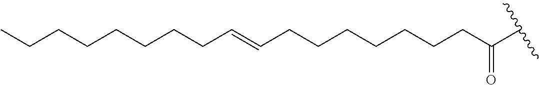 [SMILES: *C(=O)CCCCCCC/C=C/CCCCCCCC] |
| Phenyl | 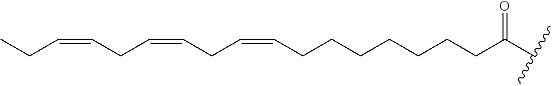 [SMILES: *C(=O)CCCCCCC/C=C\C/C=C\C/C=C\CC] |
| Phenyl | tBu |
| Phenyl | phenyl |
| Phenyl | 4-chlorophenyl |
| Phenyl | 3,4-dichlorophenyl |
| Phenyl | 4-methylphenyl |
| Phenyl | 4-methoxyphenyl |
| 4-chlorophenyl | Me |
| 4-chlorophenyl | —C₇H₁₅ |
| 4-chlorophenyl | —C₉H₁₉ |
| 4-chlorophenyl | —C₁₁H₂₃ |
| 4-chlorophenyl | —C₁₃H₂₇ |
| 4-chlorophenyl | —C₁₅H₃₁ |
| 4-chlorophenyl | —C₁₇H₃₅ |
| 4-chlorophenyl | 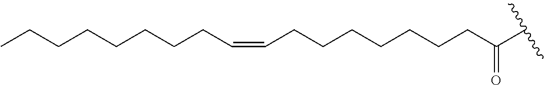 [SMILES: *C(=O)CCCCCCC/C=C\CCCCCCCC] |
| 4-chlorophenyl | 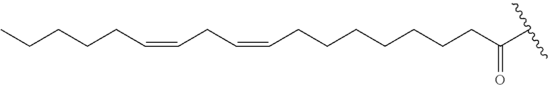 [SMILES: *C(=O)CCCCCCC/C=C\C/C=C\CCCCC] |
| 4-chlorophenyl | 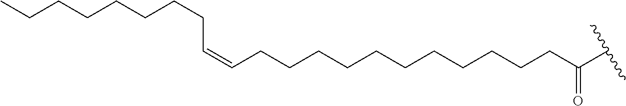 [SMILES: *C(=O)CCCCCCCCCCC/C=C\CCCCCCCC] |
| 4-chlorophenyl | 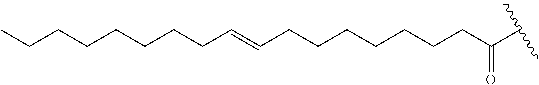 [SMILES: *C(=O)CCCCCCC/C=C/CCCCCCCC] |
| 4-chlorophenyl | 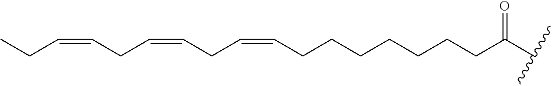 [SMILES: *C(=O)CCCCCCC/C=C\C/C=C\C/C=C\CC] |
| 4-chlorophenyl | tBu |
| 4-chlorophenyl | phenyl |
| 4-chlorophenyl | 4-chlorophenyl |
| 4-chlorophenyl | 3,4-dichlorophenyl |
| 4-chlorophenyl | 4-methylphenyl |
| 4-chlorophenyl | 4-methoxyphenyl |
| 3,4-dichlorophenyl | Me |
| 3,4-dichlorophenyl | —C₇H₁₅ |
| 3,4-dichlorophenyl | —C₉H₁₉ |
| 3,4-dichlorophenyl | —C₁₁H₂₃ |
| 3,4-dichlorophenyl | —C₁₃H₂₇ |
| 3,4-dichlorophenyl | —C₁₅H₃₁ |
| 3,4-dichlorophenyl | —C₁₇H₃₅ |
| 3,4-dichlorophenyl | 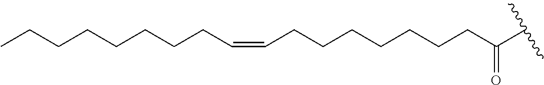 [SMILES: *C(=O)CCCCCCC/C=C\CCCCCCCC] |
| 3,4-dichlorophenyl | 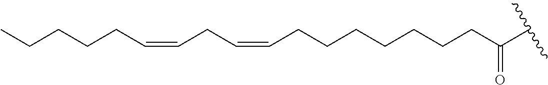 [SMILES: *C(=O)CCCCCCC/C=C\C/C=C\CCCCC] |
| 3,4-dichlorophenyl | 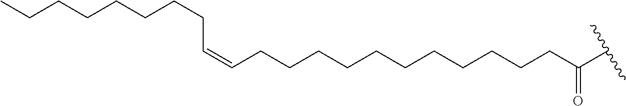 [SMILES: *C(=O)CCCCCCCCCCC/C=C\CCCCCCCC] |
| 3,4-dichlorophenyl | 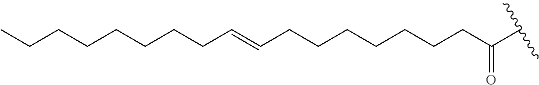 [SMILES: *C(=O)CCCCCCC/C=C/CCCCCCCC] |
| 3,4-dichlorophenyl | 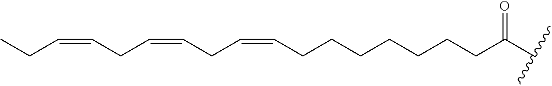 [SMILES: *C(=O)CCCCCCC/C=C\C/C=C\C/C=C\CC] |
| 3,4-dichlorophenyl | tBu |
| 3,4-dichlorophenyl | phenyl |
| 3,4-dichlorophenyl | 4-chlorophenyl |
| 3,4-dichlorophenyl | 3,4-dichlorophenyl |
| 3,4-dichlorophenyl | 4-methylphenyl |
| 3,4-dichlorophenyl | 4-methoxyphenyl |
| 4-methylphenyl | Me |
| 4-methylphenyl | —C₇H₁₅ |
| 4-methylphenyl | —C₉H₁₉ |
| 4-methylphenyl | —C₁₁H₂₃ |
| 4-methylphenyl | —C₁₃H₂₇ |
| 4-methylphenyl | —C₁₅H₃₁ |
| 4-methylphenyl | —C₁₇H₃₅ |
| 4-methylphenyl | 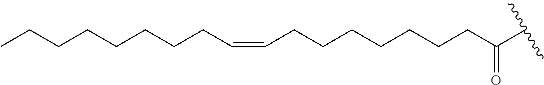 [SMILES: *C(=O)CCCCCCC/C=C\CCCCCCCC] |
| 4-methylphenyl | 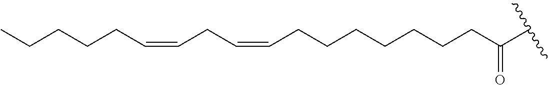 [SMILES: *C(=O)CCCCCCC/C=C\C/C=C\CCCCC] |
| 4-methylphenyl | 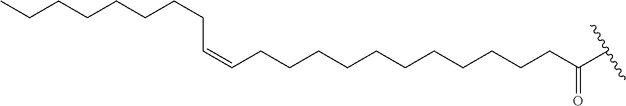 [SMILES: *C(=O)CCCCCCCCCCC/C=C\CCCCCCCC] |
| 4-methylphenyl | 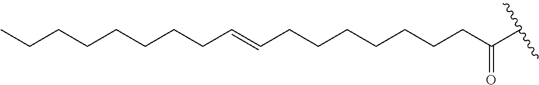 [SMILES: *C(=O)CCCCCCC/C=C/CCCCCCCC] |
| 4-methylphenyl | 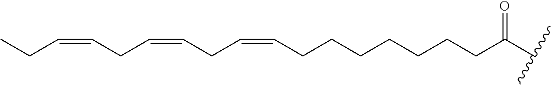 [SMILES: *C(=O)CCCCCCC/C=C\C/C=C\C/C=C\CC] |
| 4-methylphenyl | tBu |
| 4-methylphenyl | phenyl |
| 4-methylphenyl | 4-chlorophenyl |
| 4-methylphenyl | 3,4-dichlorophenyl |
| 4-methylphenyl | 4-methylphenyl |
| 4-methylphenyl | 4-methoxyphenyl |
| 4-methoxyphenyl | Me |
| 4-methoxyphenyl | —C₇H₁₅ |
| 4-methoxyphenyl | —C₉H₁₉ |
| 4-methoxyphenyl | —C₁₁H₂₃ |
| 4-methoxyphenyl | —C₁₃H₂₇ |
| 4-methoxyphenyl | —C₁₅H₃₁ |
| 4-methoxyphenyl | —C₁₇H₃₅ |
| 4-methoxyphenyl | 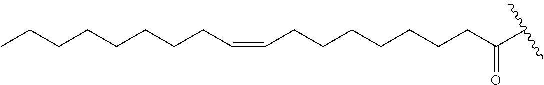 [SMILES: *C(=O)CCCCCCC/C=C\CCCCCCCC] |
| 4-methoxyphenyl | 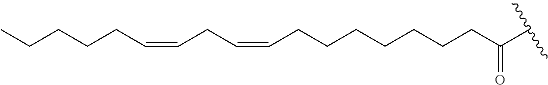 [SMILES: *C(=O)CCCCCCC/C=C\C/C=C\CCCCC] |
| 4-methoxyphenyl | 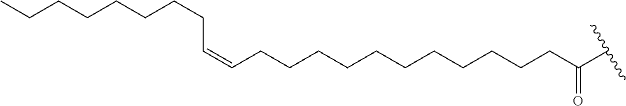 [SMILES: *C(=O)CCCCCCCCCCC/C=C\CCCCCCCC] |
| 4-methoxyphenyl | 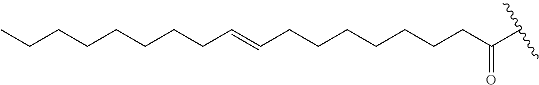 [SMILES: *C(=O)CCCCCCC/C=C/CCCCCCCC] |
| 4-methoxyphenyl | 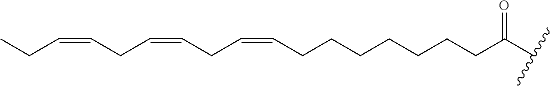 [SMILES: *C(=O)CCCCCCC/C=C\C/C=C\C/C=C\CC] |
| 4-methoxyphenyl | tBu |
| 4-methoxyphenyl | phenyl |
| 4-methoxyphenyl | 4-chlorophenyl |
| 4-methoxyphenyl | 3,4-dichlorophenyl |
| 4-methoxyphenyl | 4-methylphenyl |
| 4-methoxyphenyl | 4-methoxyphenyl |
| 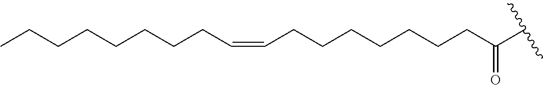 [SMILES: *C(=O)CCCCCCC/C=C\CCCCCCCC] | Me |
| 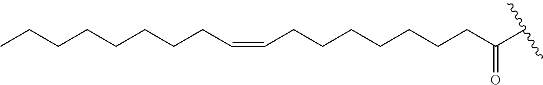 [SMILES: *C(=O)CCCCCCC/C=C\CCCCCCCC] | —C₇H₁₅ |
| 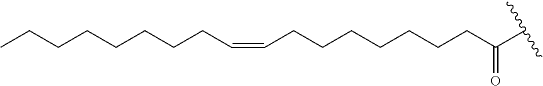 [SMILES: *C(=O)CCCCCCC/C=C\CCCCCCCC] | —C₉H₁₉ |
| 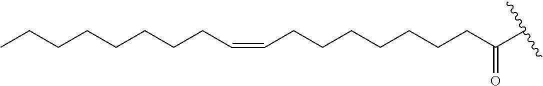 [SMILES: *C(=O)CCCCCCC/C=C\CCCCCCCC] | —C₁₁H₂₃ |
| 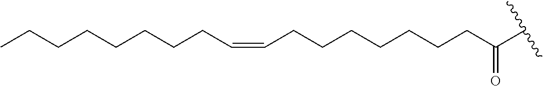 [SMILES: *C(=O)CCCCCCC/C=C\CCCCCCCC] | —C₁₃H₂₇ |
| 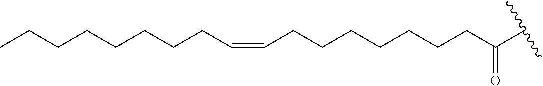 [SMILES: *C(=O)CCCCCCC/C=C\CCCCCCCC] | —C₁₅H₃₁ |
| 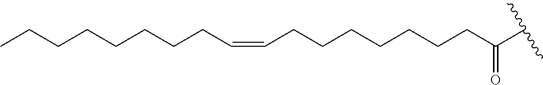 [SMILES: *C(=O)CCCCCCC/C=C\CCCCCCCC] | —C₁₇H₃₅ |
| 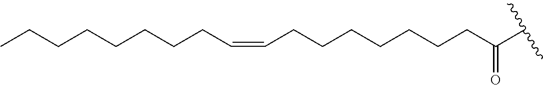 [SMILES: *C(=O)CCCCCCC/C=C\CCCCCCCC] | 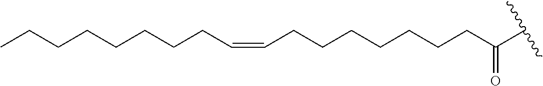 [SMILES: *C(=O)CCCCCCC/C=C\CCCCCCCC] |
| 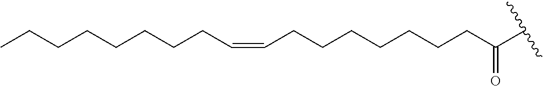 [SMILES: *C(=O)CCCCCCC/C=C\CCCCCCCC] | 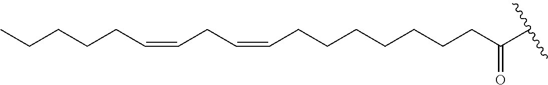 [SMILES: *C(=O)CCCCCCC/C=C\C/C=C\CCCCC] |
| 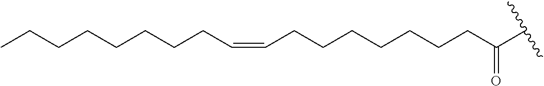 [SMILES: *C(=O)CCCCCCC/C=C\CCCCCCCC] | 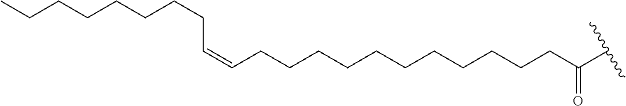 [SMILES: *C(=O)CCCCCCCCCCC/C=C\CCCCCCCC] |
| 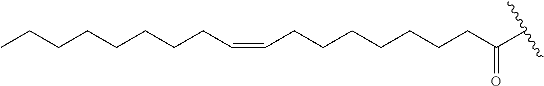 [SMILES: *C(=O)CCCCCCC/C=C\CCCCCCCC] | 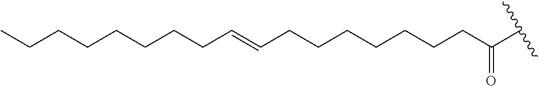 [SMILES: *C(=O)CCCCCCC/C=C/CCCCCCCC] |
| 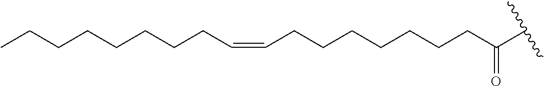 [SMILES: *C(=O)CCCCCCC/C=C\CCCCCCCC] | 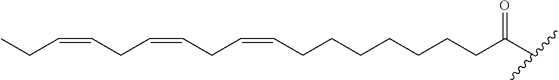 [SMILES: *C(=O)CCCCCCC/C=C\C/C=C\C/C=C\CC] |
| 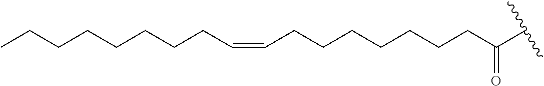 [SMILES: *C(=O)CCCCCCC/C=C\CCCCCCCC] | tBu |
| 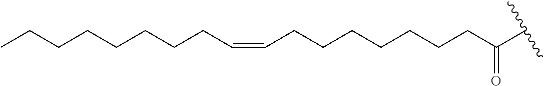 [SMILES: *C(=O)CCCCCCC/C=C\CCCCCCCC] | phenyl |
| 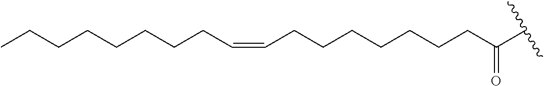 [SMILES: *C(=O)CCCCCCC/C=C\CCCCCCCC] | 4-chlorophenyl |
| 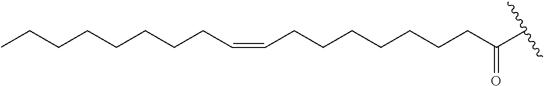 [SMILES: *C(=O)CCCCCCC/C=C\CCCCCCCC] | 3,4-dichlorophenyl |
| 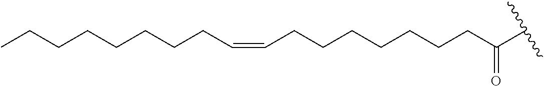 [SMILES: *C(=O)CCCCCCC/C=C\CCCCCCCC] | 4-methylphenyl |
| 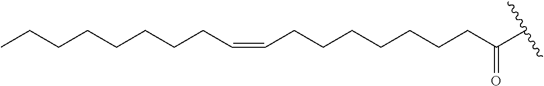 [SMILES: *C(=O)CCCCCCC/C=C\CCCCCCCC] | 4-methoxyphenyl |
| 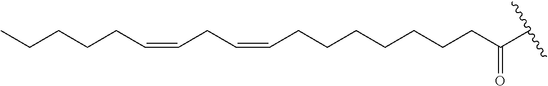 [SMILES: *C(=O)CCCCCCC/C=C\C/C=C\CCCCC] | Me |
| 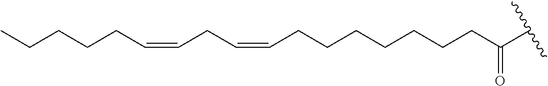 [SMILES: *C(=O)CCCCCCC/C=C\C/C=C\CCCCC] | —C₇H₁₅ |
| 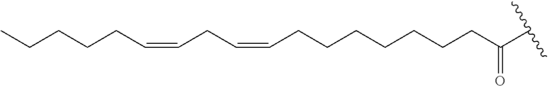 [SMILES: *C(=O)CCCCCCC/C=C\C/C=C\CCCCC] | —C₉H₁₉ |
| 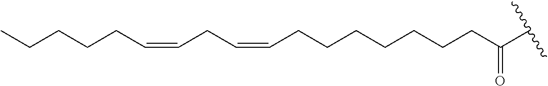 [SMILES: *C(=O)CCCCCCC/C=C\C/C=C\CCCCC] | —C₁₁H₂₃ |
| 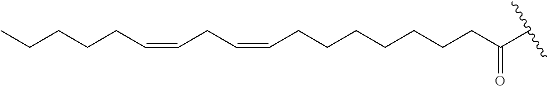 [SMILES: *C(=O)CCCCCCC/C=C\C/C=C\CCCCC] | —C₁₃H₂₇ |
| 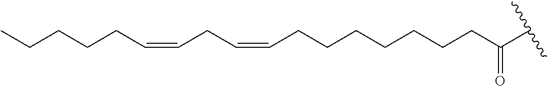 [SMILES: *C(=O)CCCCCCC/C=C\C/C=C\CCCCC] | —C₁₅H₃₁ |
| 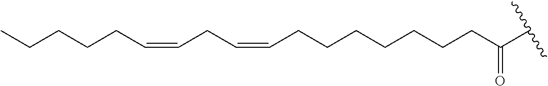 [SMILES: *C(=O)CCCCCCC/C=C\C/C=C\CCCCC] | —C₁₇H₃₅ |
| 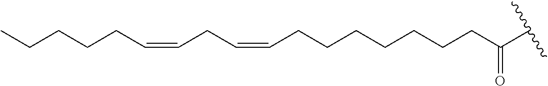 [SMILES: *C(=O)CCCCCCC/C=C\C/C=C\CCCCC] | 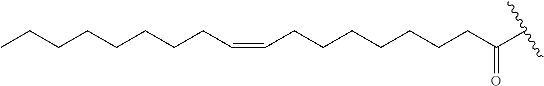 [SMILES: *C(=O)CCCCCCC/C=C\CCCCCCCC] |
| 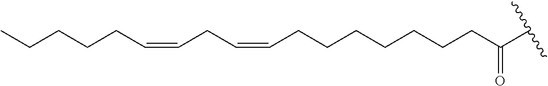 [SMILES: *C(=O)CCCCCCC/C=C\C/C=C\CCCCC] | 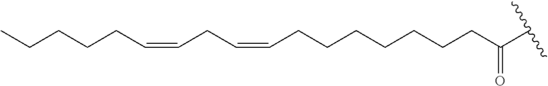 [SMILES: *C(=O)CCCCCCC/C=C\C/C=C\CCCCC] |
| 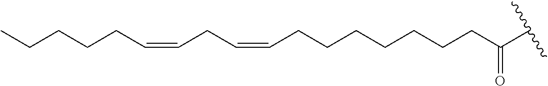 [SMILES: *C(=O)CCCCCCC/C=C\C/C=C\CCCCC] | 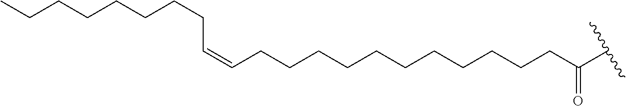 [SMILES: *C(=O)CCCCCCCCCCC/C=C\CCCCCCCC] |
| 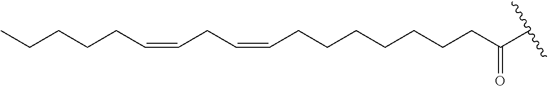 [SMILES: *C(=O)CCCCCCC/C=C\C/C=C\CCCCC] | 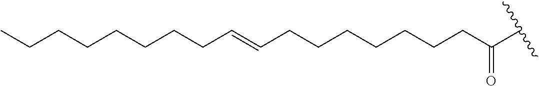 [SMILES: *C(=O)CCCCCCC/C=C/CCCCCCCC] |
| 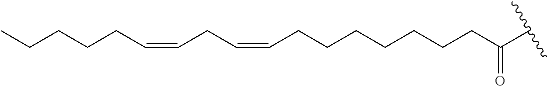 [SMILES: *C(=O)CCCCCCC/C=C\C/C=C\CCCCC] | 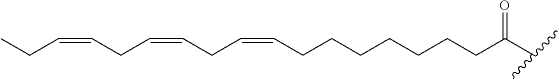 [SMILES: *C(=O)CCCCCCC/C=C\C/C=C\C/C=C\CC] |
| 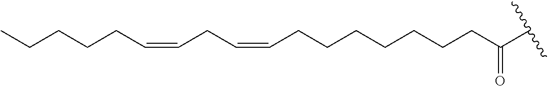 [SMILES: *C(=O)CCCCCCC/C=C\C/C=C\CCCCC] | tBu |
| 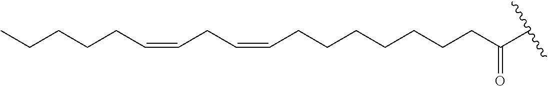 [SMILES: *C(=O)CCCCCCC/C=C\C/C=C\CCCCC] | phenyl |
| 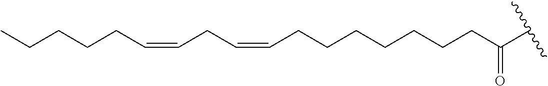 [SMILES: *C(=O)CCCCCCC/C=C\C/C=C\CCCCC] | 4-chlorophenyl |
| 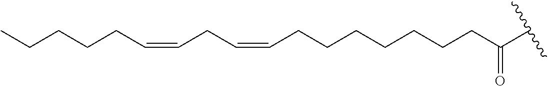 [SMILES: *C(=O)CCCCCCC/C=C\C/C=C\CCCCC] | 3,4-dichlorophenyl |
| 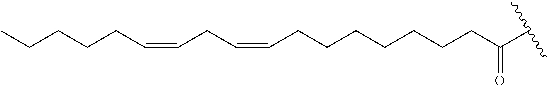 [SMILES: *C(=O)CCCCCCC/C=C\C/C=C\CCCCC] | 4-methylphenyl |
| 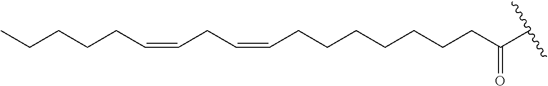 [SMILES: *C(=O)CCCCCCC/C=C\C/C=C\CCCCC] | 4-methoxyphenyl |
| 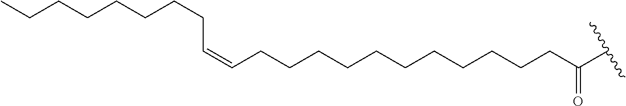 [SMILES: *C(=O)CCCCCCCCCCC/C=C\CCCCCCCC] | Me |
| 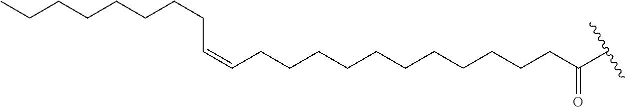 [SMILES: *C(=O)CCCCCCCCCCC/C=C\CCCCCCCC] | —C₇H₁₅ |
| 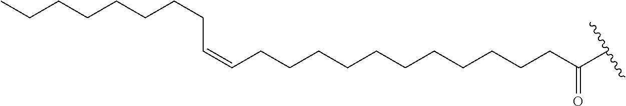 [SMILES: *C(=O)CCCCCCCCCCC/C=C\CCCCCCCC] | —C₉H₁₉ |
| 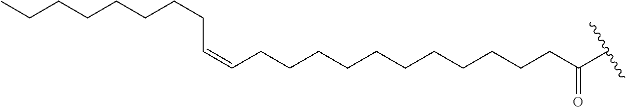 [SMILES: *C(=O)CCCCCCCCCCC/C=C\CCCCCCCC] | —C₁₁H₂₃ |
| 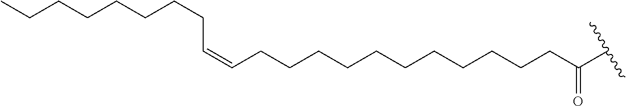 [SMILES: *C(=O)CCCCCCCCCCC/C=C\CCCCCCCC] | —C₁₃H₂₇ |
| 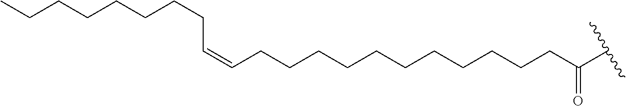 [SMILES: *C(=O)CCCCCCCCCCC/C=C\CCCCCCCC] | —C₁₅H₃₁ |
| 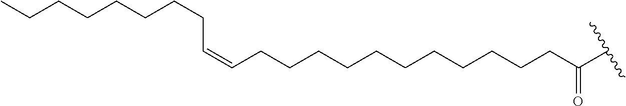 [SMILES: *C(=O)CCCCCCCCCCC/C=C\CCCCCCCC] | —C₁₇H₃₅ |
| 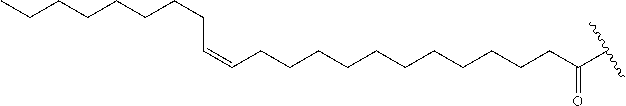 [SMILES: *C(=O)CCCCCCCCCCC/C=C\CCCCCCCC] | 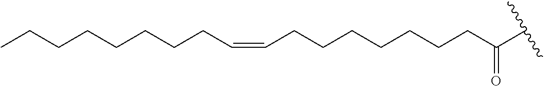 [SMILES: *C(=O)CCCCCCC/C=C\CCCCCCCC] |
| 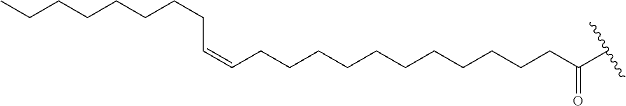 [SMILES: *C(=O)CCCCCCCCCCC/C=C\CCCCCCCC] | 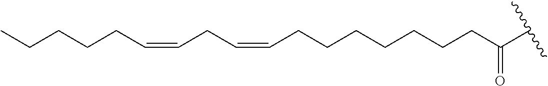 [SMILES: *C(=O)CCCCCCC/C=C\C/C=C\CCCCC] |
| 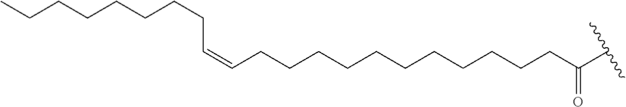 [SMILES: *C(=O)CCCCCCCCCCC/C=C\CCCCCCCC] | 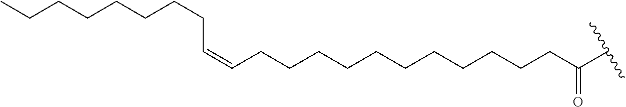 [SMILES: *C(=O)CCCCCCCCCCC/C=C\CCCCCCCC] |
| 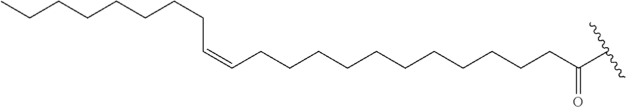 [SMILES: *C(=O)CCCCCCCCCCC/C=C\CCCCCCCC] | 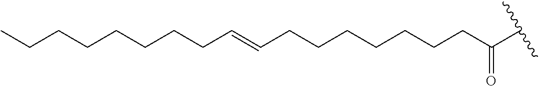 [SMILES: *C(=O)CCCCCCC/C=C/CCCCCCCC] |
| 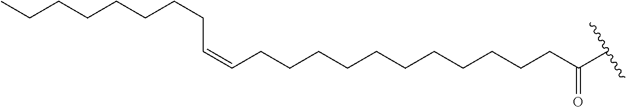 [SMILES: *C(=O)CCCCCCCCCCC/C=C\CCCCCCCC] | 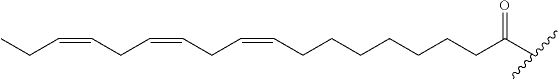 [SMILES: *C(=O)CCCCCCC/C=C\C/C=C\C/C=C\CC] |
| 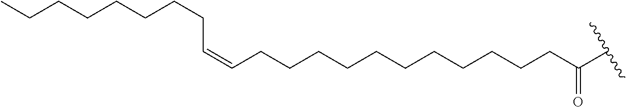 [SMILES: *C(=O)CCCCCCCCCCC/C=C\CCCCCCCC] | tBu |
| 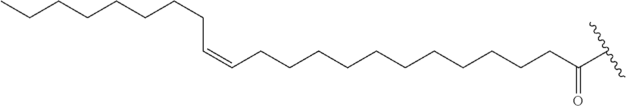 [SMILES: *C(=O)CCCCCCCCCCC/C=C\CCCCCCCC] | phenyl |
| 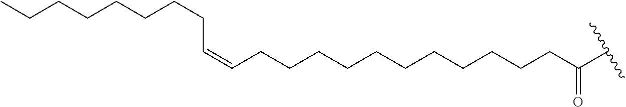 [SMILES: *C(=O)CCCCCCCCCCC/C=C\CCCCCCCC] | 4-chlorophenyl |
| 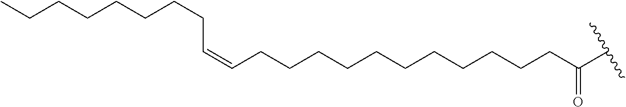 [SMILES: *C(=O)CCCCCCCCCCC/C=C\CCCCCCCC] | 3,4-dichlorophenyl |
| 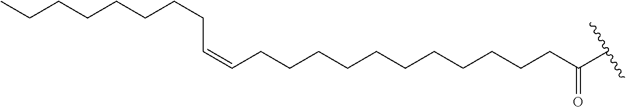 [SMILES: *C(=O)CCCCCCCCCCC/C=C\CCCCCCCC] | 4-methylphenyl |
| 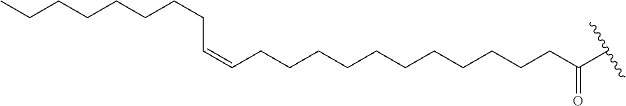 [SMILES: *C(=O)CCCCCCCCCCC/C=C\CCCCCCCC] | 4-methoxyphenyl |
| 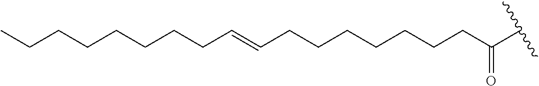 [SMILES: *C(=O)CCCCCCC/C=C/CCCCCCCC] | Me |
| 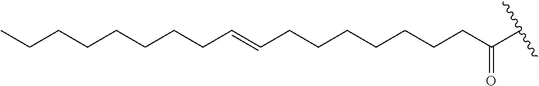 [SMILES: *C(=O)CCCCCCC/C=C/CCCCCCCC] | —C₇H₁₅ |
| 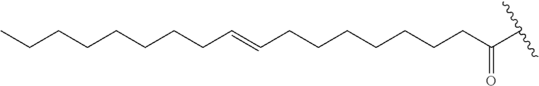 [SMILES: *C(=O)CCCCCCC/C=C/CCCCCCCC] | —C₉H₁₉ |
| 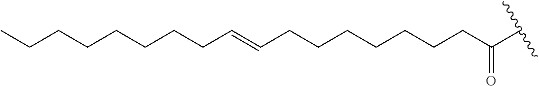 [SMILES: *C(=O)CCCCCCC/C=C/CCCCCCCC] | —C₁₁H₂₃ |
| 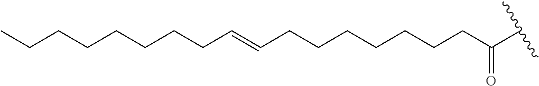 [SMILES: *C(=O)CCCCCCC/C=C/CCCCCCCC] | —C₁₃H₂₇ |
| 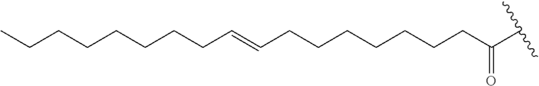 [SMILES: *C(=O)CCCCCCC/C=C/CCCCCCCC] | —C₁₅H₃₁ |
| 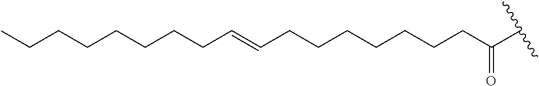 [SMILES: *C(=O)CCCCCCC/C=C/CCCCCCCC] | —C₁₇H₃₅ |
| 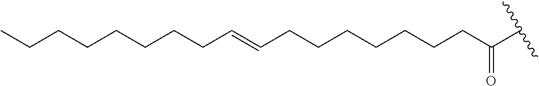 [SMILES: *C(=O)CCCCCCC/C=C/CCCCCCCC] | 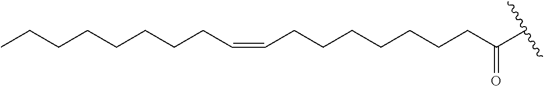 [SMILES: *C(=O)CCCCCCC/C=C\CCCCCCCC] |
| 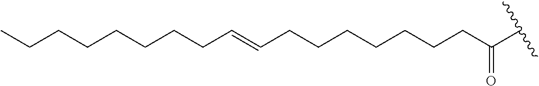 [SMILES: *C(=O)CCCCCCC/C=C/CCCCCCCC] | 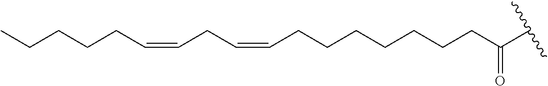 [SMILES: *C(=O)CCCCCCC/C=C\C/C=C\CCCCC] |
| 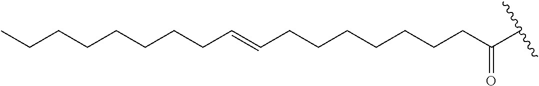 [SMILES: *C(=O)CCCCCCC/C=C/CCCCCCCC] | 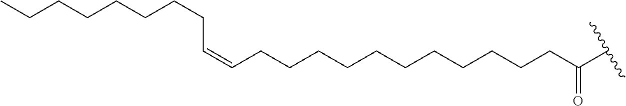 [SMILES: *C(=O)CCCCCCCCCCC/C=C\CCCCCCCC] |
| 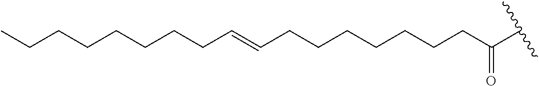 [SMILES: *C(=O)CCCCCCC/C=C/CCCCCCCC] | 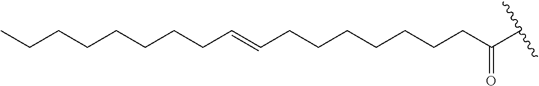 [SMILES: *C(=O)CCCCCCC/C=C/CCCCCCCC] |
| 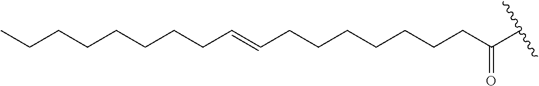 [SMILES: *C(=O)CCCCCCC/C=C/CCCCCCCC] | 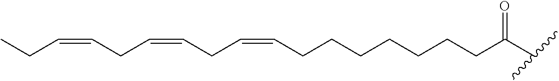 [SMILES: *C(=O)CCCCCCC/C=C\C/C=C\C/C=C\CC] |
| 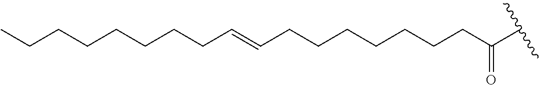 [SMILES: *C(=O)CCCCCCC/C=C/CCCCCCCC] | tBu |
| 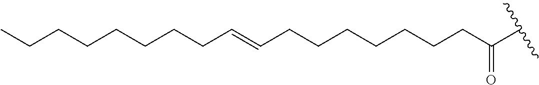 [SMILES: *C(=O)CCCCCCC/C=C/CCCCCCCC] | phenyl |
| 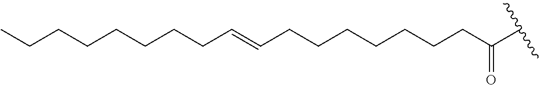 [SMILES: *C(=O)CCCCCCC/C=C/CCCCCCCC] | 4-chlorophenyl |
| 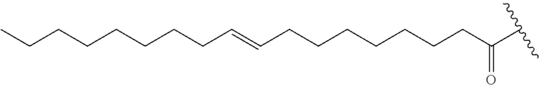 [SMILES: *C(=O)CCCCCCC/C=C/CCCCCCCC] | 3,4-dichlorophenyl |
| 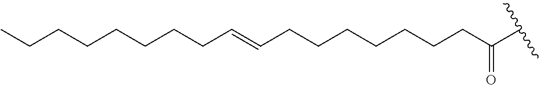 [SMILES: *C(=O)CCCCCCC/C=C/CCCCCCCC] | 4-methylphenyl |
| 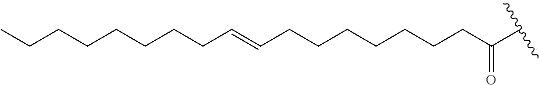 [SMILES: *C(=O)CCCCCCC/C=C/CCCCCCCC] | 4-methoxyphenyl |
| 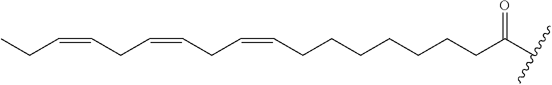 [SMILES: *C(=O)CCCCCCC/C=C\C/C=C\C/C=C\CC] | Me |
| 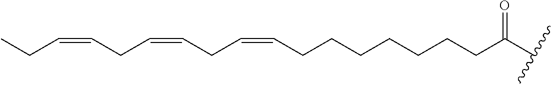 [SMILES: *C(=O)CCCCCCC/C=C\C/C=C\C/C=C\CC] | —C₇H₁₅ |
| 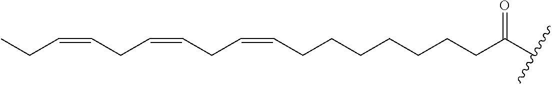 [SMILES: *C(=O)CCCCCCC/C=C\C/C=C\C/C=C\CC] | —C₉H₁₉ |
| 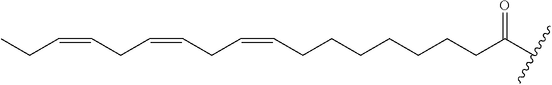 [SMILES: *C(=O)CCCCCCC/C=C\C/C=C\C/C=C\CC] | —C₁₁H₂₃ |
| 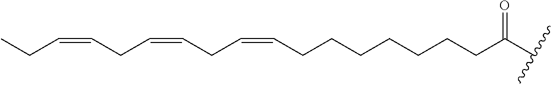 [SMILES: *C(=O)CCCCCCC/C=C\C/C=C\C/C=C\CC] | —C₁₃H₂₇ |
| 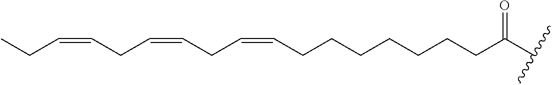 [SMILES: *C(=O)CCCCCCC/C=C\C/C=C\C/C=C\CC] | —C₁₅H₃₁ |
| 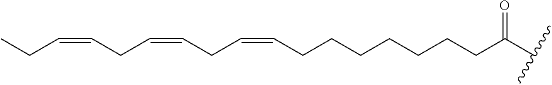 [SMILES: *C(=O)CCCCCCC/C=C\C/C=C\C/C=C\CC] | —C₁₇H₃₅ |
| 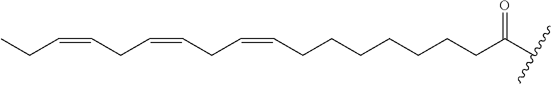 [SMILES: *C(=O)CCCCCCC/C=C\C/C=C\C/C=C\CC] | 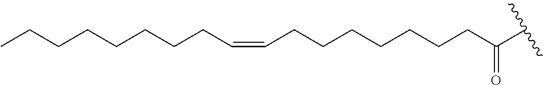 [SMILES: *C(=O)CCCCCCC/C=C\CCCCCCCC] |
| 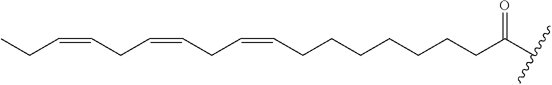 [SMILES: *C(=O)CCCCCCC/C=C\C/C=C\C/C=C\CC] | 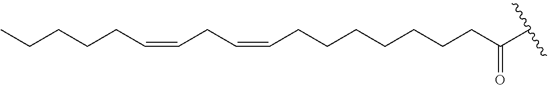 [SMILES: *C(=O)CCCCCCC/C=C\C/C=C\CCCCC] |
| 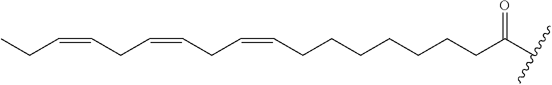 [SMILES: *C(=O)CCCCCCC/C=C\C/C=C\C/C=C\CC] | 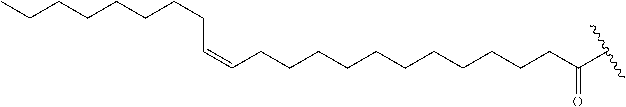 [SMILES: *C(=O)CCCCCCCCCCC/C=C\CCCCCCCC] |
| 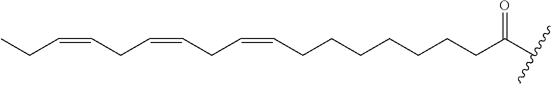 [SMILES: *C(=O)CCCCCCC/C=C\C/C=C\C/C=C\CC] | 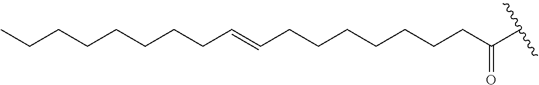 [SMILES: *C(=O)CCCCCCC/C=C/CCCCCCCC] |
| 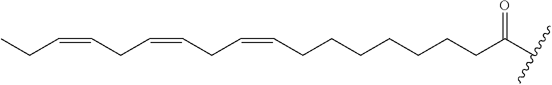 [SMILES: *C(=O)CCCCCCC/C=C\C/C=C\C/C=C\CC] | 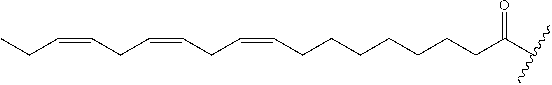 [SMILES: *C(=O)CCCCCCC/C=C\C/C=C\C/C=C\CC] |
| 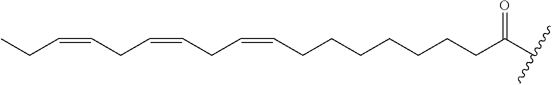 [SMILES: *C(=O)CCCCCCC/C=C\C/C=C\C/C=C\CC] | tBu |
| 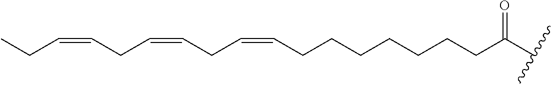 [SMILES: *C(=O)CCCCCCC/C=C\C/C=C\C/C=C\CC] | phenyl |
| 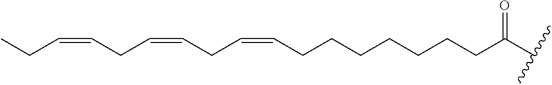 [SMILES: *C(=O)CCCCCCC/C=C\C/C=C\C/C=C\CC] | 4-chlorophenyl |
| 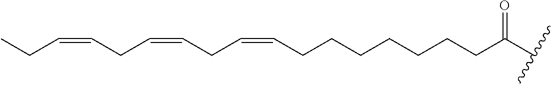 [SMILES: *C(=O)CCCCCCC/C=C\C/C=C\C/C=C\CC] | 3,4-dichlorophenyl |
| 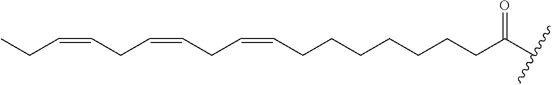 [SMILES: *C(=O)CCCCCCC/C=C\C/C=C\C/C=C\CC] | 4-methylphenyl |
| 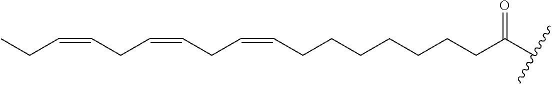 [SMILES: *C(=O)CCCCCCC/C=C\C/C=C\C/C=C\CC] | 4-methoxyphenyl |
| H | 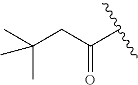 [SMILES: *C(=O)CC(C)(C)C] |
| H | 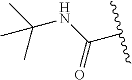 [SMILES: *C(=O)NC(C)(C)C] |
| H | 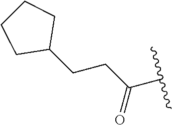 [SMILES: *C(=O)CCC1CCCC1] |
| H | 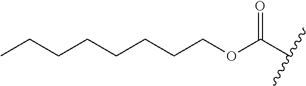 [SMILES: *C(=O)OCCCCCCCC] |
| H | 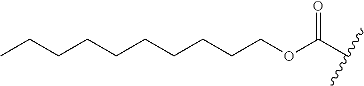 [SMILES: *C(=O)OCCCCCCCCCC] |
| H | 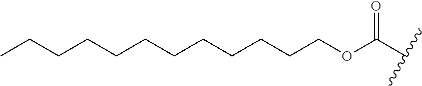 [SMILES: *C(=O)OCCCCCCCCCCCC] |
| H | 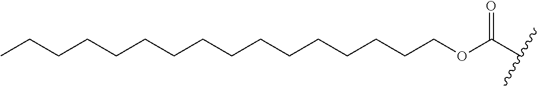 [SMILES: *C(=O)OCCCCCCCCCCCCCCCC] |
| H | 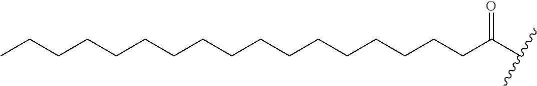 [SMILES: *C(=O)CCCCCCCCCCCCCCCCC] |
| H | 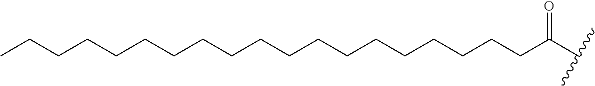 [SMILES: *C(=O)CCCCCCCCCCCCCCCCCCC] |
| H | 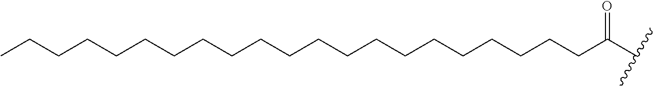 [SMILES: *C(=O)CCCCCCCCCCCCCCCCCCCCC] |
| H | 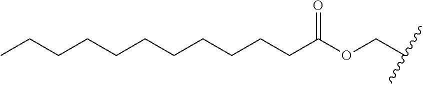 [SMILES: *COC(=O)CCCCCCCCCCC] |
| H | 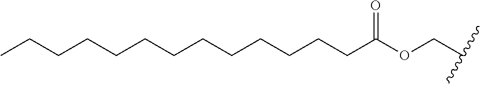 [SMILES: *COC(=O)CCCCCCCCCCCCC] |
| H | 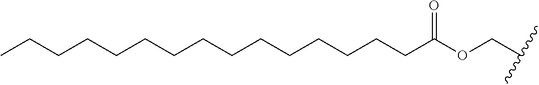 [SMILES: *COC(=O)CCCCCCCCCCCCCCC] |
| H | 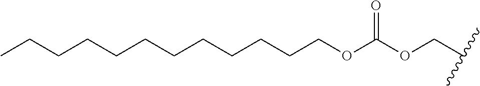 [SMILES: *COC(=O)OCCCCCCCCCCCC] |
| H | 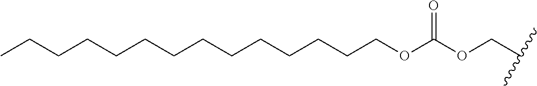 [SMILES: *COC(=O)OCCCCCCCCCCCCCC] |
| H | 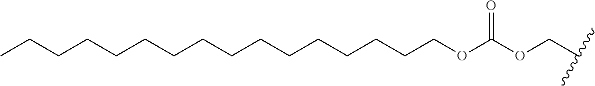 [SMILES: *COC(=O)OCCCCCCCCCCCCCCCC] |
| Me | 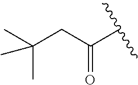 [SMILES: *C(=O)CC(C)(C)C] |
| Me | 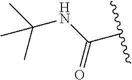 [SMILES: *C(=O)NC(C)(C)C] |
| Me | 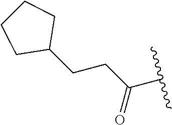 [SMILES: *C(=O)CCC1CCCC1] |
| Me | 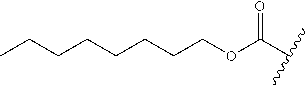 [SMILES: *C(=O)OCCCCCCCC] |
| Me | 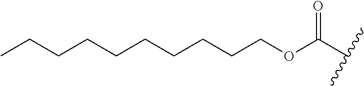 [SMILES: *C(=O)OCCCCCCCCCC] |
| Me | 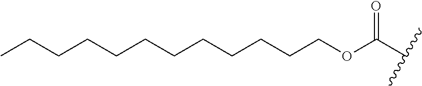 [SMILES: *C(=O)OCCCCCCCCCCCC] |
| Me | 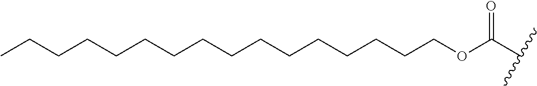 [SMILES: *C(=O)OCCCCCCCCCCCCCCCC] |
| Me | 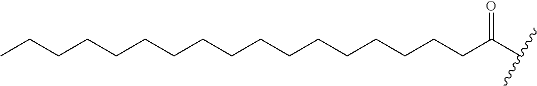 [SMILES: *C(=O)CCCCCCCCCCCCCCCCC] |
| Me | 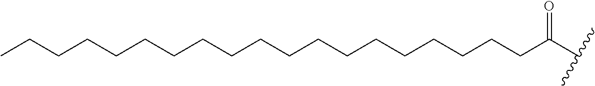 [SMILES: *C(=O)CCCCCCCCCCCCCCCCCCC] |
| Me | 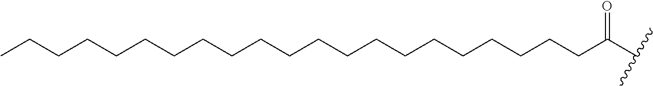 [SMILES: *C(=O)CCCCCCCCCCCCCCCCCCCCC] |
| Me | 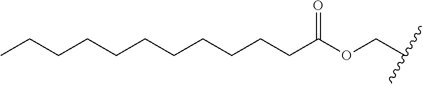 [SMILES: *COC(=O)CCCCCCCCCCC] |
| Me | 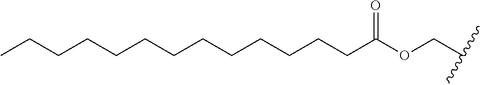 [SMILES: *COC(=O)CCCCCCCCCCCCC] |
| Me | 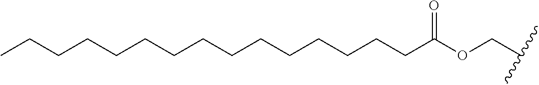 [SMILES: *COC(=O)CCCCCCCCCCCCCCC] |
| Me | 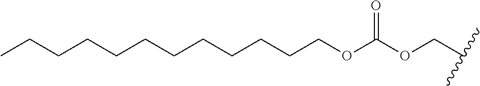 [SMILES: *COC(=O)OCCCCCCCCCCCC] |
| Me | 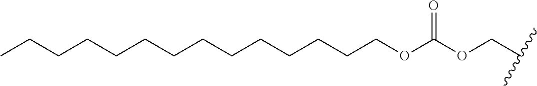 [SMILES: *COC(=O)OCCCCCCCCCCCCCC] |
| Me | 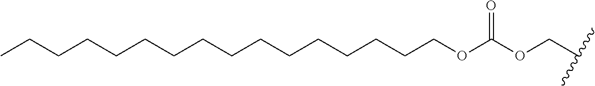 [SMILES: *COC(=O)OCCCCCCCCCCCCCCCC] |
| —C₇H₁₅ | 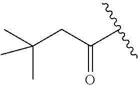 [SMILES: *C(=O)CC(C)(C)C] |
| —C₇H₁₅ | 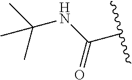 [SMILES: *C(=O)NC(C)(C)C] |
| —C₇H₁₅ | 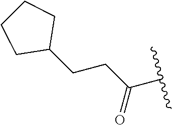 [SMILES: *C(=O)CCC1CCCC1] |
| —C₇H₁₅ | 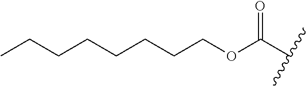 [SMILES: *C(=O)OCCCCCCCC] |
| —C₇H₁₅ | 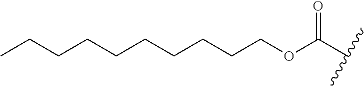 [SMILES: *C(=O)OCCCCCCCCCC] |
| —C₇H₁₅ | 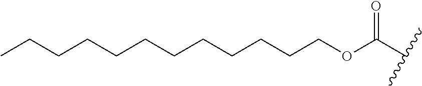 [SMILES: *C(=O)OCCCCCCCCCCCC] |
| —C₇H₁₅ | 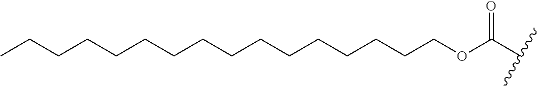 [SMILES: *C(=O)OCCCCCCCCCCCCCCCC] |
| —C₇H₁₅ | 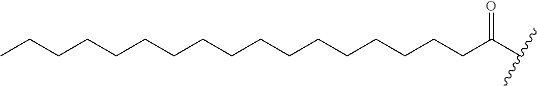 [SMILES: *C(=O)CCCCCCCCCCCCCCCCC] |
| —C₇H₁₅ | 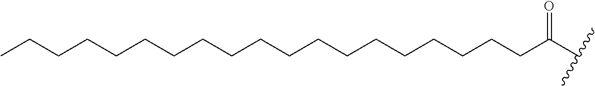 [SMILES: *C(=O)CCCCCCCCCCCCCCCCCCC] |
| —C₇H₁₅ | 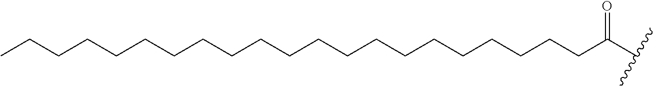 [SMILES: *C(=O)CCCCCCCCCCCCCCCCCCCCC] |
| —C₇H₁₅ | 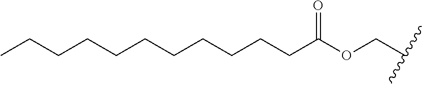 [SMILES: *COC(=O)CCCCCCCCCCC] |
| —C₇H₁₅ | 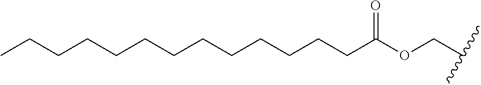 [SMILES: *COC(=O)CCCCCCCCCCCCC] |
| —C₇H₁₅ | 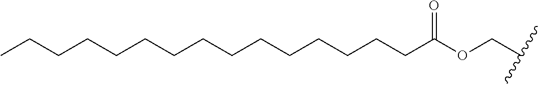 [SMILES: *COC(=O)CCCCCCCCCCCCCCC] |
| —C₇H₁₅ | 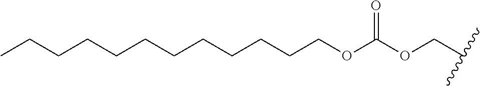 [SMILES: *COC(=O)OCCCCCCCCCCCC] |
| —C₇H₁₅ | 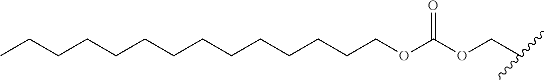 [SMILES: *COC(=O)OCCCCCCCCCCCCCC] |
| —C₇H₁₅ | 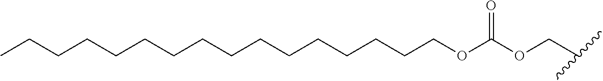 [SMILES: *COC(=O)OCCCCCCCCCCCCCCCC] |
| —C₉H₁₉ | 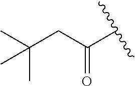 [SMILES: *C(=O)CC(C)(C)C] |
| —C₉H₁₉ | 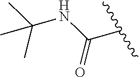 [SMILES: *C(=O)NC(C)(C)C] |
| —C₉H₁₉ | 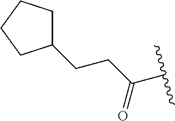 [SMILES: *C(=O)CCC1CCCC1] |
| —C₉H₁₉ | 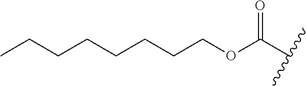 [SMILES: *C(=O)OCCCCCCCC] |
| —C₉H₁₉ | 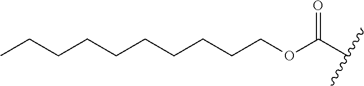 [SMILES: *C(=O)OCCCCCCCCCC] |
| —C₉H₁₉ | 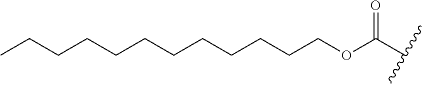 [SMILES: *C(=O)OCCCCCCCCCCCC] |
| —C₉H₁₉ | 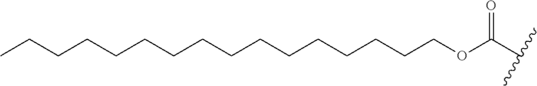 [SMILES: *C(=O)OCCCCCCCCCCCCCCCC] |
| —C₉H₁₉ | 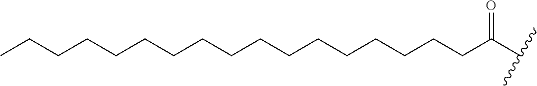 [SMILES: *C(=O)CCCCCCCCCCCCCCCCC] |
| —C₉H₁₉ | 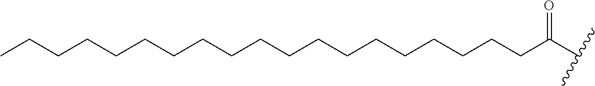 [SMILES: *C(=O)CCCCCCCCCCCCCCCCCCC] |
| —C₉H₁₉ | 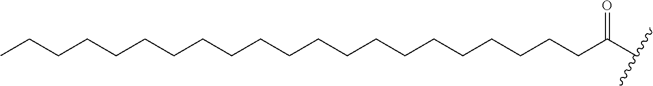 [SMILES: *C(=O)CCCCCCCCCCCCCCCCCCCCC] |
| —C₉H₁₉ | 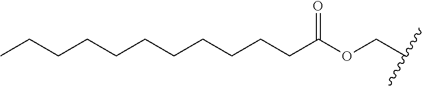 [SMILES: *COC(=O)CCCCCCCCCCC] |
| —C₉H₁₉ | 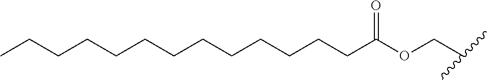 [SMILES: *COC(=O)CCCCCCCCCCCCC] |
| —C₉H₁₉ | 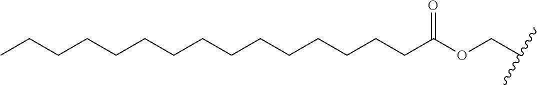 [SMILES: *COC(=O)CCCCCCCCCCCCCCC] |
| —C₉H₁₉ | 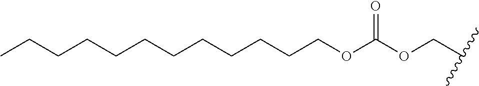 [SMILES: *COC(=O)OCCCCCCCCCCCC] |
| —C₉H₁₉ | 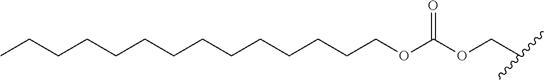 [SMILES: *COC(=O)OCCCCCCCCCCCCCC] |
| —C₉H₁₉ | 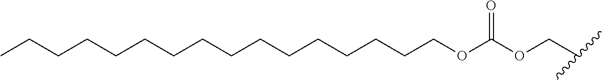 [SMILES: *COC(=O)OCCCCCCCCCCCCCCCC] |
| —C₁₁H₂₃ | 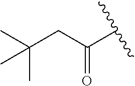 [SMILES: *C(=O)CC(C)(C)C] |
| —C₁₁H₂₃ | 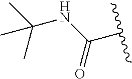 [SMILES: *C(=O)NC(C)(C)C] |
| —C₁₁H₂₃ | 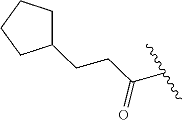 [SMILES: *C(=O)CCC1CCCC1] |
| —C₁₁H₂₃ | 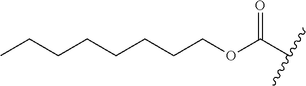 [SMILES: *C(=O)OCCCCCCCC] |
| —C₁₁H₂₃ | 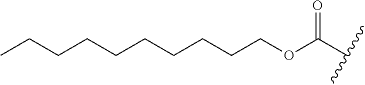 [SMILES: *C(=O)OCCCCCCCCCC] |
| —C₁₁H₂₃ | 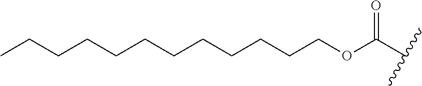 [SMILES: *C(=O)OCCCCCCCCCCCC] |
| —C₁₁H₂₃ | 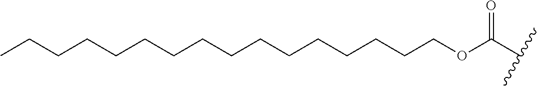 [SMILES: *C(=O)OCCCCCCCCCCCCCCCC] |
| —C₁₁H₂₃ | 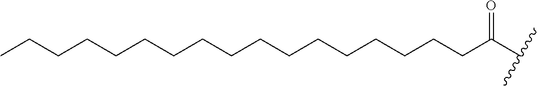 [SMILES: *C(=O)CCCCCCCCCCCCCCCCC] |
| —C₁₁H₂₃ | 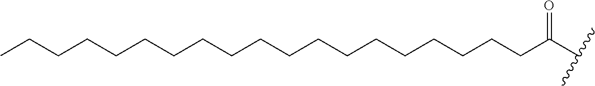 [SMILES: *C(=O)CCCCCCCCCCCCCCCCCCC] |
| —C₁₁H₂₃ | 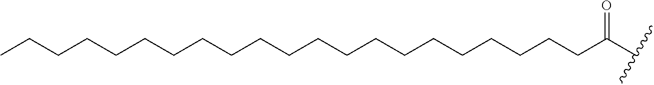 [SMILES: *C(=O)CCCCCCCCCCCCCCCCCCCCC] |
| —C₁₁H₂₃ | 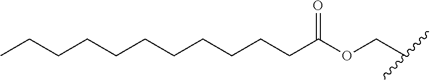 [SMILES: *COC(=O)CCCCCCCCCCC] |
| —C₁₁H₂₃ | 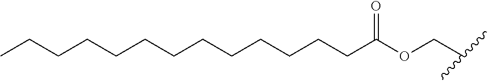 [SMILES: *COC(=O)CCCCCCCCCCCCC] |
| —C₁₁H₂₃ | 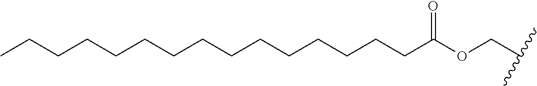 [SMILES: *COC(=O)CCCCCCCCCCCCCCC] |
| —C₁₁H₂₃ | 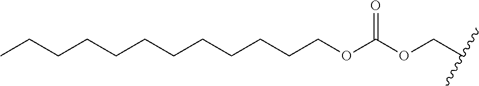 [SMILES: *COC(=O)OCCCCCCCCCCCC] |
| —C₁₁H₂₃ | 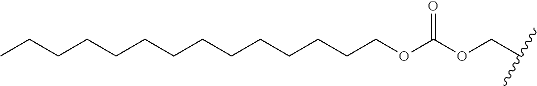 [SMILES: *COC(=O)OCCCCCCCCCCCCCC] |
| —C₁₁H₂₃ | 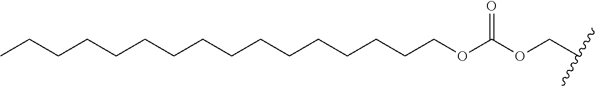 [SMILES: *COC(=O)OCCCCCCCCCCCCCCCC] |
| —C₁₃H₂₇ | 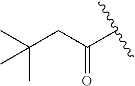 [SMILES: *C(=O)CC(C)(C)C] |
| —C₁₃H₂₇ | 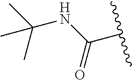 [SMILES: *C(=O)NC(C)(C)C] |
| —C₁₃H₂₇ | 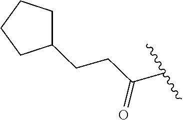 [SMILES: *C(=O)CCC1CCCC1] |
| —C₁₃H₂₇ | 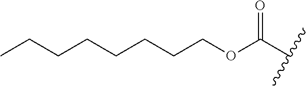 [SMILES: *C(=O)OCCCCCCCC] |
| —C₁₃H₂₇ | 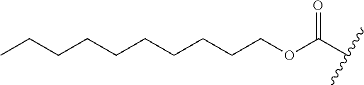 [SMILES: *C(=O)OCCCCCCCCCC] |
| —C₁₃H₂₇ | 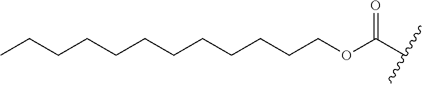 [SMILES: *C(=O)OCCCCCCCCCCCC] |
| —C₁₃H₂₇ | 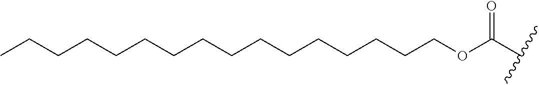 [SMILES: *C(=O)OCCCCCCCCCCCCCCCC] |
| —C₁₃H₂₇ | 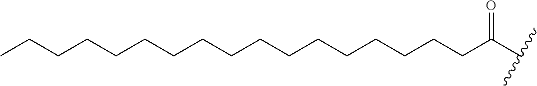 [SMILES: *C(=O)CCCCCCCCCCCCCCCCC] |
| —C₁₃H₂₇ | 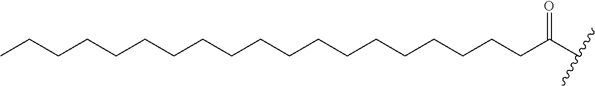 [SMILES: *C(=O)CCCCCCCCCCCCCCCCCCC] |
| —C₁₃H₂₇ | 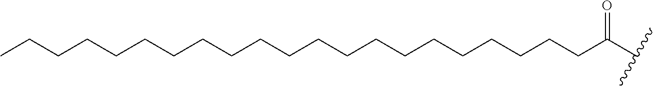 [SMILES: *C(=O)CCCCCCCCCCCCCCCCCCCCC] |
| —C₁₃H₂₇ | 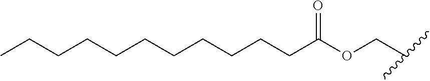 [SMILES: *COC(=O)CCCCCCCCCCC] |
| —C₁₃H₂₇ | 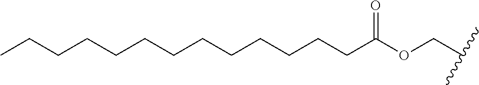 [SMILES: *COC(=O)CCCCCCCCCCCCC] |
| —C₁₃H₂₇ | 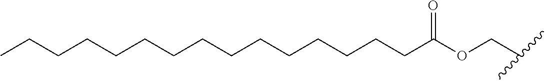 [SMILES: *COC(=O)CCCCCCCCCCCCCCC] |
| —C₁₃H₂₇ | 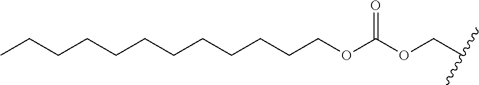 [SMILES: *COC(=O)OCCCCCCCCCCCC] |
| —C₁₃H₂₇ | 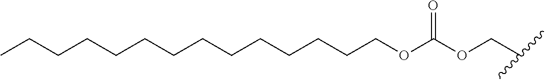 [SMILES: *COC(=O)OCCCCCCCCCCCCCC] |
| —C₁₃H₂₇ | 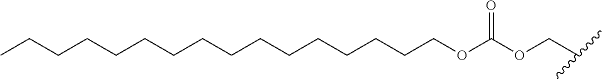 [SMILES: *COC(=O)OCCCCCCCCCCCCCCCC] |
| —C₁₅H₃₁ | 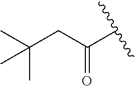 [SMILES: *C(=O)CC(C)(C)C] |
| —C₁₅H₃₁ | 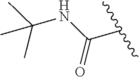 [SMILES: *C(=O)NC(C)(C)C] |
| —C₁₅H₃₁ | 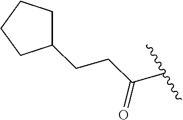 [SMILES: *C(=O)CCC1CCCC1] |
| —C₁₅H₃₁ | 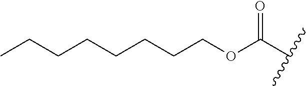 [SMILES: *C(=O)OCCCCCCCC] |
| —C₁₅H₃₁ | 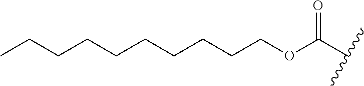 [SMILES: *C(=O)OCCCCCCCCCC] |
| —C₁₅H₃₁ | 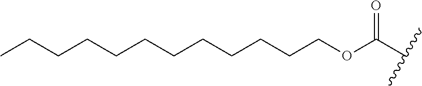 [SMILES: *C(=O)OCCCCCCCCCCCC] |
| —C₁₅H₃₁ | 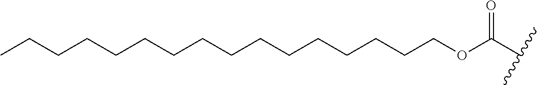 [SMILES: *C(=O)OCCCCCCCCCCCCCCCC] |
| —C₁₅H₃₁ | 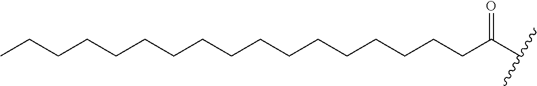 [SMILES: *C(=O)CCCCCCCCCCCCCCCCC] |
| —C₁₅H₃₁ | 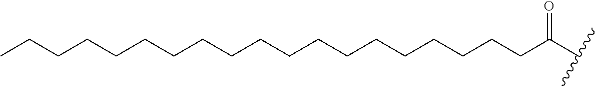 [SMILES: *C(=O)CCCCCCCCCCCCCCCCCCC] |
| —C₁₅H₃₁ | 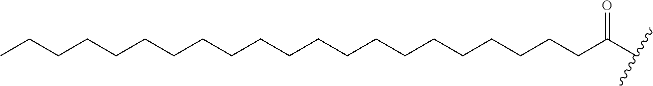 [SMILES: *C(=O)CCCCCCCCCCCCCCCCCCCCC] |
| —C₁₅H₃₁ | 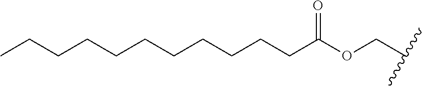 [SMILES: *COC(=O)CCCCCCCCCCC] |
| —C₁₅H₃₁ | 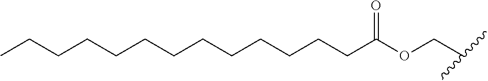 [SMILES: *COC(=O)CCCCCCCCCCCCC] |
| —C₁₅H₃₁ | 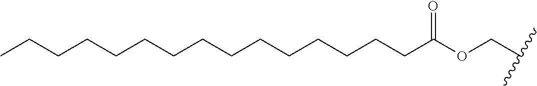 [SMILES: *COC(=O)CCCCCCCCCCCCCCC] |
| —C₁₅H₃₁ | 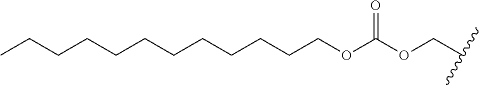 [SMILES: *COC(=O)OCCCCCCCCCCCC] |
| —C₁₅H₃₁ | 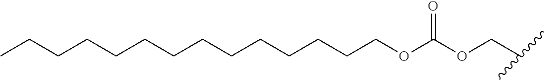 [SMILES: *COC(=O)OCCCCCCCCCCCCCC] |
| —C₁₅H₃₁ | 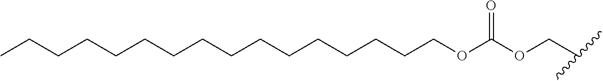 [SMILES: *COC(=O)OCCCCCCCCCCCCCCCC] |
| —C₁₇H₃₅ | 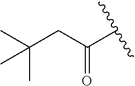 [SMILES: *C(=O)CC(C)(C)C] |
| —C₁₇H₃₅ | 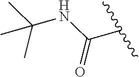 [SMILES: *C(=O)NC(C)(C)C] |
| —C₁₇H₃₅ | 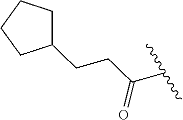 [SMILES: *C(=O)CCC1CCCC1] |
| —C₁₇H₃₅ | 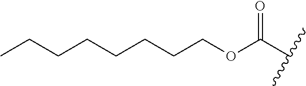 [SMILES: *C(=O)OCCCCCCCC] |
| —C₁₇H₃₅ | 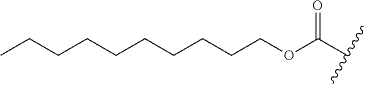 [SMILES: *C(=O)OCCCCCCCCCC] |
| —C₁₇H₃₅ | 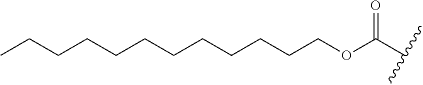 [SMILES: *C(=O)OCCCCCCCCCCCC] |
| —C₁₇H₃₅ | 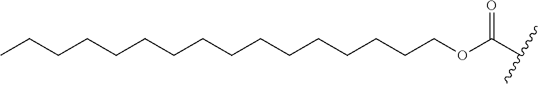 [SMILES: *C(=O)OCCCCCCCCCCCCCCCC] |
| —C₁₇H₃₅ | 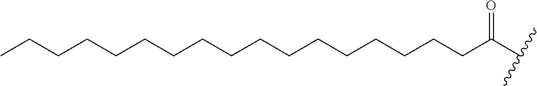 [SMILES: *C(=O)CCCCCCCCCCCCCCCCC] |
| —C₁₇H₃₅ | 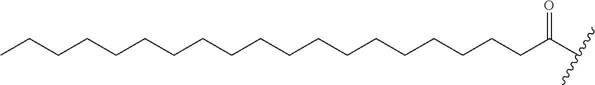 [SMILES: *C(=O)CCCCCCCCCCCCCCCCCCC] |
| —C₁₇H₃₅ | 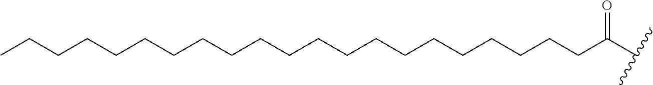 [SMILES: *C(=O)CCCCCCCCCCCCCCCCCCCCC] |
| —C₁₇H₃₅ | 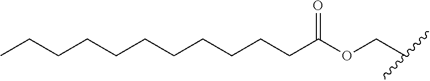 [SMILES: *COC(=O)CCCCCCCCCCC] |
| —C₁₇H₃₅ | 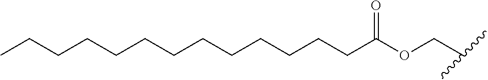 [SMILES: *COC(=O)CCCCCCCCCCCCC] |
| —C₁₇H₃₅ | 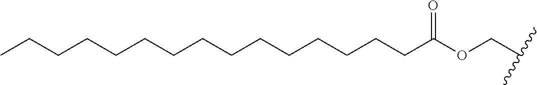 [SMILES: *COC(=O)CCCCCCCCCCCCCCC] |
| —C₁₇H₃₅ | 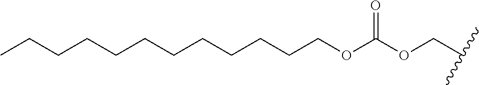 [SMILES: *COC(=O)OCCCCCCCCCCCC] |
| —C₁₇H₃₅ | 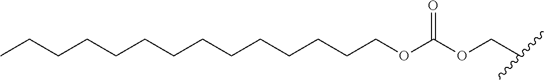 [SMILES: *COC(=O)OCCCCCCCCCCCCCC] |
| —C₁₇H₃₅ | 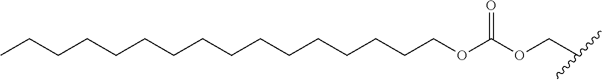 [SMILES: *COC(=O)OCCCCCCCCCCCCCCCC] |
| 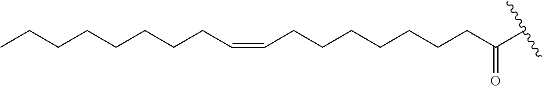 [SMILES: *C(=O)CCCCCCC/C=C\CCCCCCCC] | 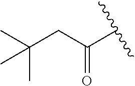 [SMILES: *C(=O)CC(C)(C)C] |
| 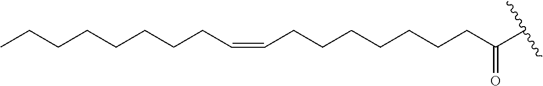 [SMILES: *C(=O)CCCCCCC/C=C\CCCCCCCC] | 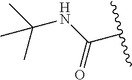 [SMILES: *C(=O)NC(C)(C)C] |
| 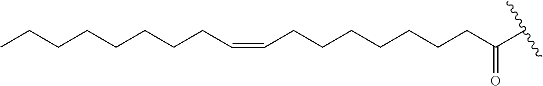 [SMILES: *C(=O)CCCCCCC/C=C\CCCCCCCC] | 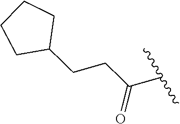 [SMILES: *C(=O)CCC1CCCC1] |
| 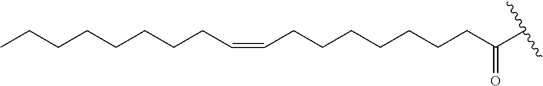 [SMILES: *C(=O)CCCCCCC/C=C\CCCCCCCC] | 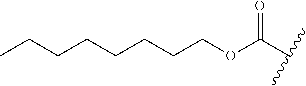 [SMILES: *C(=O)OCCCCCCCC] |
| 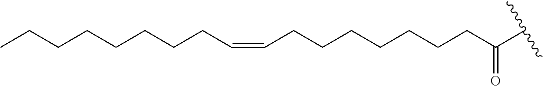 [SMILES: *C(=O)CCCCCCC/C=C\CCCCCCCC] | 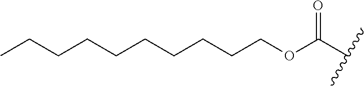 [SMILES: *C(=O)OCCCCCCCCCC] |
| 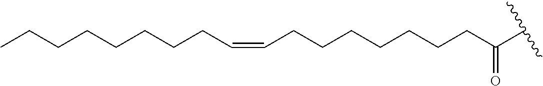 [SMILES: *C(=O)CCCCCCC/C=C\CCCCCCCC] | 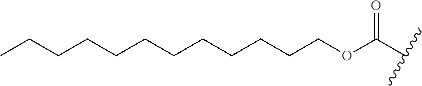 [SMILES: *C(=O)OCCCCCCCCCCCC] |
| 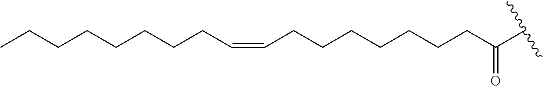 [SMILES: *C(=O)CCCCCCC/C=C\CCCCCCCC] | 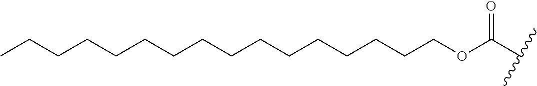 [SMILES: *C(=O)OCCCCCCCCCCCCCCCC] |
| 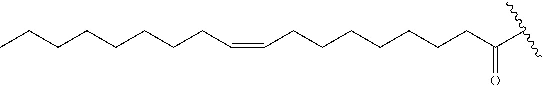 [SMILES: *C(=O)CCCCCCC/C=C\CCCCCCCC] | 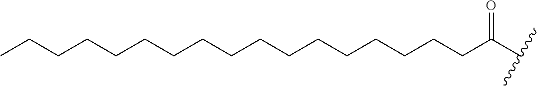 [SMILES: *C(=O)CCCCCCCCCCCCCCCCC] |
| 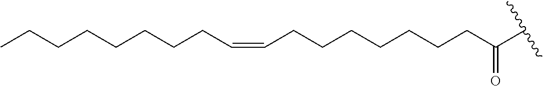 [SMILES: *C(=O)CCCCCCC/C=C\CCCCCCCC] | 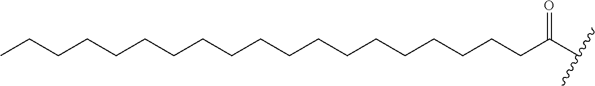 [SMILES: *C(=O)CCCCCCCCCCCCCCCCCCC] |
| 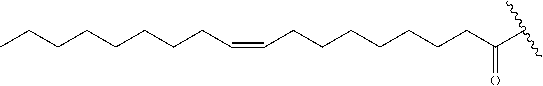 [SMILES: *C(=O)CCCCCCC/C=C\CCCCCCCC] | 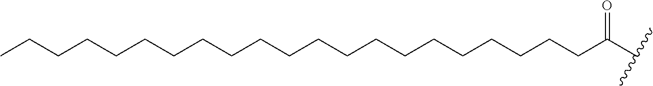 [SMILES: *C(=O)CCCCCCCCCCCCCCCCCCCCC] |
| 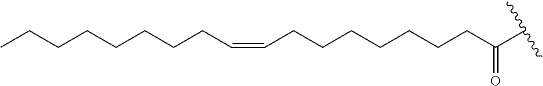 [SMILES: *C(=O)CCCCCCC/C=C\CCCCCCCC] | 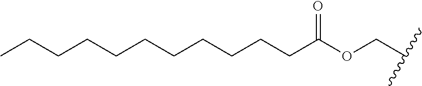 [SMILES: *COC(=O)CCCCCCCCCCC] |
| 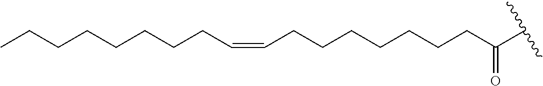 [SMILES: *C(=O)CCCCCCC/C=C\CCCCCCCC] | 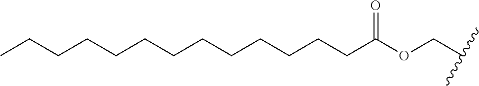 [SMILES: *COC(=O)CCCCCCCCCCCCC] |
| 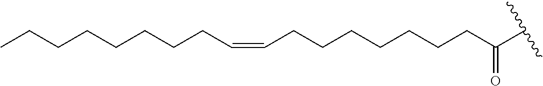 [SMILES: *C(=O)CCCCCCC/C=C\CCCCCCCC] | 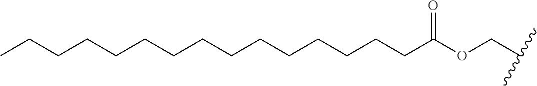 [SMILES: *COC(=O)CCCCCCCCCCCCCCC] |
| 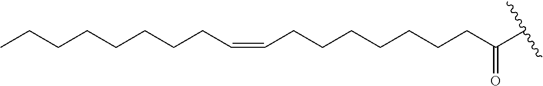 [SMILES: *C(=O)CCCCCCC/C=C\CCCCCCCC] | 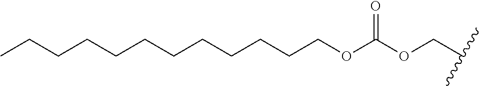 [SMILES: *COC(=O)OCCCCCCCCCCCC] |
| 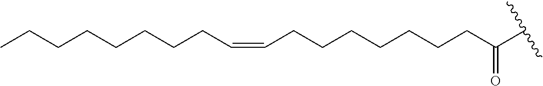 [SMILES: *C(=O)CCCCCCC/C=C\CCCCCCCC] | 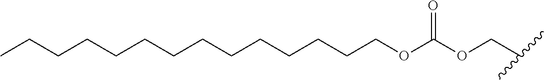 [SMILES: *COC(=O)OCCCCCCCCCCCCCC] |
| 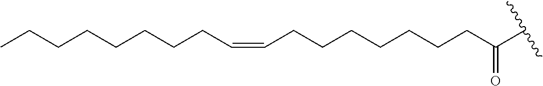 [SMILES: *C(=O)CCCCCCC/C=C\CCCCCCCC] | 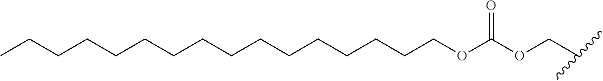 [SMILES: *COC(=O)OCCCCCCCCCCCCCCCC] |
| 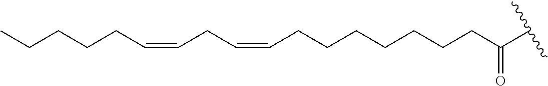 [SMILES: *C(=O)CCCCCCC/C=C\C/C=C\CCCCC] | 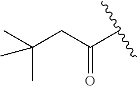 [SMILES: *C(=O)CC(C)(C)C] |
| 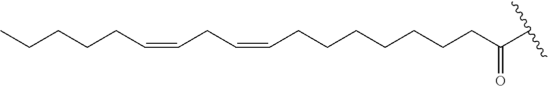 [SMILES: *C(=O)CCCCCCC/C=C\C/C=C\CCCCC] | 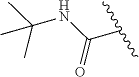 [SMILES: *C(=O)NC(C)(C)C] |
| 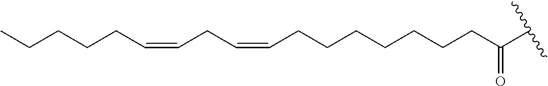 [SMILES: *C(=O)CCCCCCC/C=C\C/C=C\CCCCC] | 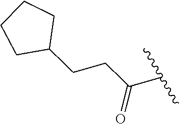 [SMILES: *C(=O)CCC1CCCC1] |
| 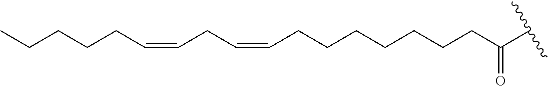 [SMILES: *C(=O)CCCCCCC/C=C\C/C=C\CCCCC] | 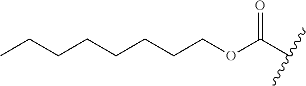 [SMILES: *C(=O)OCCCCCCCC] |
| 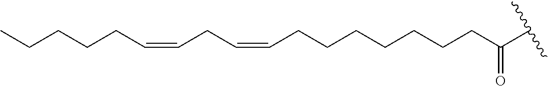 [SMILES: *C(=O)CCCCCCC/C=C\C/C=C\CCCCC] | 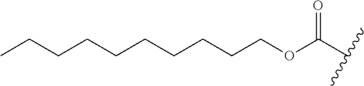 [SMILES: *C(=O)OCCCCCCCCCC] |
| 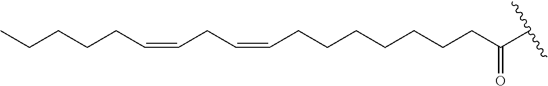 [SMILES: *C(=O)CCCCCCC/C=C\C/C=C\CCCCC] | 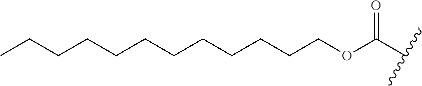 [SMILES: *C(=O)OCCCCCCCCCCCC] |
| 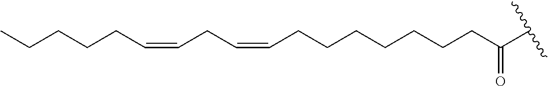 [SMILES: *C(=O)CCCCCCC/C=C\C/C=C\CCCCC] | 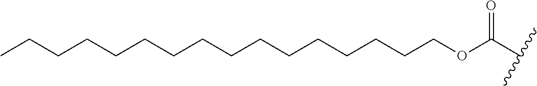 [SMILES: *C(=O)OCCCCCCCCCCCCCCCC] |
| 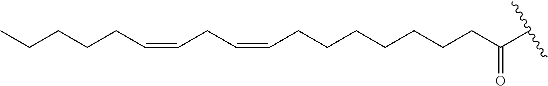 [SMILES: *C(=O)CCCCCCC/C=C\C/C=C\CCCCC] | 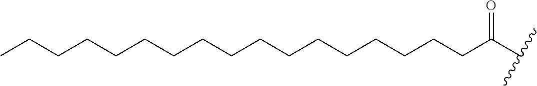 [SMILES: *C(=O)CCCCCCCCCCCCCCCCC] |
| 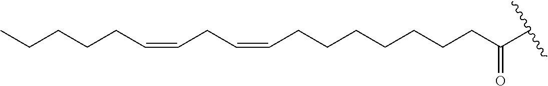 [SMILES: *C(=O)CCCCCCC/C=C\C/C=C\CCCCC] | 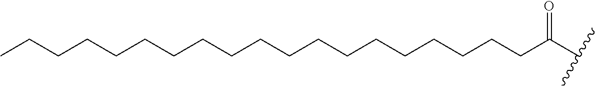 [SMILES: *C(=O)CCCCCCCCCCCCCCCCCCC] |
| 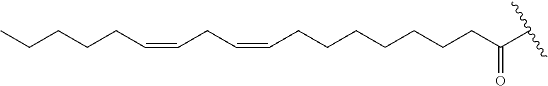 [SMILES: *C(=O)CCCCCCC/C=C\C/C=C\CCCCC] | 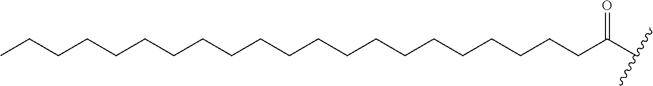 [SMILES: *C(=O)CCCCCCCCCCCCCCCCCCCCC] |
| 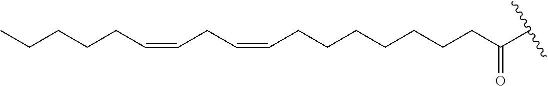 [SMILES: *C(=O)CCCCCCC/C=C\C/C=C\CCCCC] | 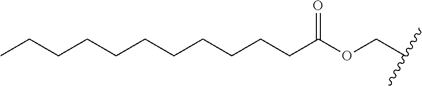 [SMILES: *COC(=O)CCCCCCCCCCC] |
| 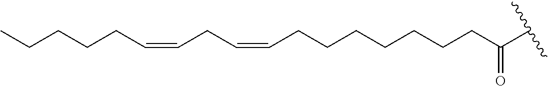 [SMILES: *C(=O)CCCCCCC/C=C\C/C=C\CCCCC] | 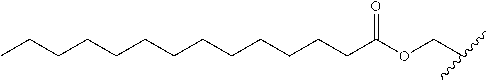 [SMILES: *COC(=O)CCCCCCCCCCCCC] |
| 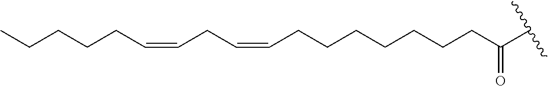 [SMILES: *C(=O)CCCCCCC/C=C\C/C=C\CCCCC] | 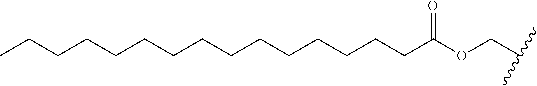 [SMILES: *COC(=O)CCCCCCCCCCCCCCC] |
| 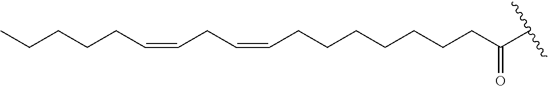 [SMILES: *C(=O)CCCCCCC/C=C\C/C=C\CCCCC] | 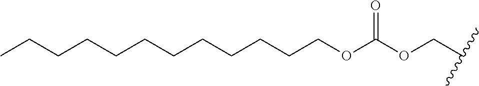 [SMILES: *COC(=O)OCCCCCCCCCCCC] |
| 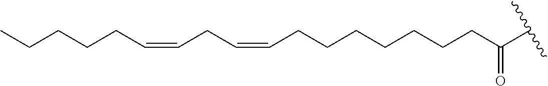 [SMILES: *C(=O)CCCCCCC/C=C\C/C=C\CCCCC] | 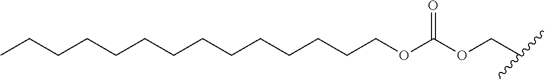 [SMILES: *COC(=O)OCCCCCCCCCCCCCC] |
| 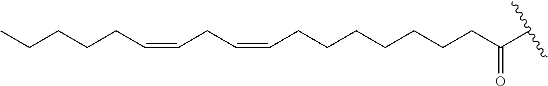 [SMILES: *C(=O)CCCCCCC/C=C\C/C=C\CCCCC] | 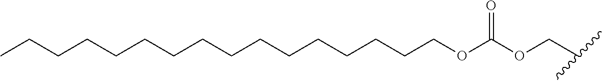 [SMILES: *COC(=O)OCCCCCCCCCCCCCCCC] |
| 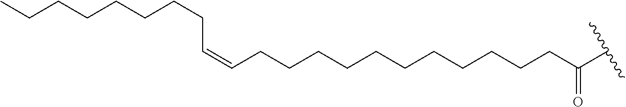 [SMILES: *C(=O)CCCCCCCCCCC/C=C\CCCCCCCC] | 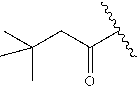 [SMILES: *C(=O)CC(C)(C)C] |
| 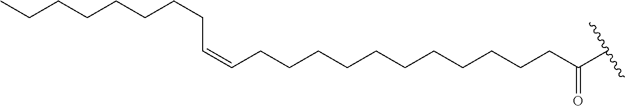 [SMILES: *C(=O)CCCCCCCCCCC/C=C\CCCCCCCC] | 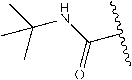 [SMILES: *C(=O)NC(C)(C)C] |
| 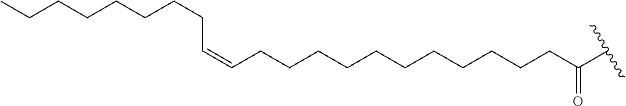 [SMILES: *C(=O)CCCCCCCCCCC/C=C\CCCCCCCC] | 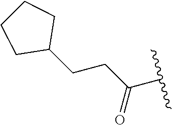 [SMILES: *C(=O)CCC1CCCC1] |
| 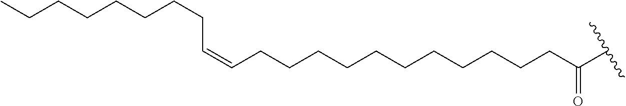 [SMILES: *C(=O)CCCCCCCCCCC/C=C\CCCCCCCC] | 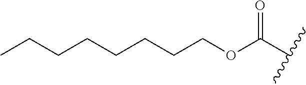 [SMILES: *C(=O)OCCCCCCCC] |
| 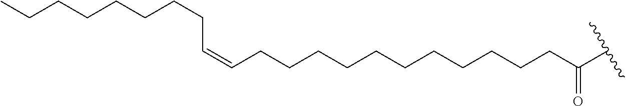 [SMILES: *C(=O)CCCCCCCCCCC/C=C\CCCCCCCC] | 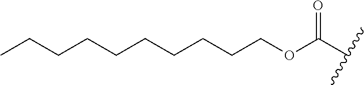 [SMILES: *C(=O)OCCCCCCCCCC] |
| 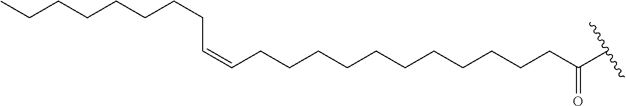 [SMILES: *C(=O)CCCCCCCCCCC/C=C\CCCCCCCC] | 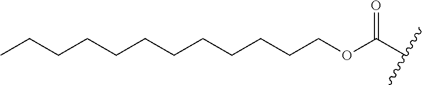 [SMILES: *C(=O)OCCCCCCCCCCCC] |
| 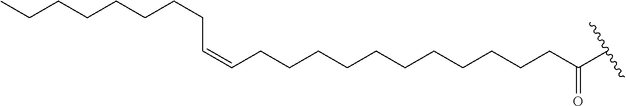 [SMILES: *C(=O)CCCCCCCCCCC/C=C\CCCCCCCC] | 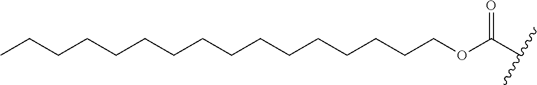 [SMILES: *C(=O)OCCCCCCCCCCCCCCCC] |
| 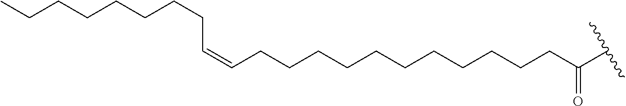 [SMILES: *C(=O)CCCCCCCCCCC/C=C\CCCCCCCC] | 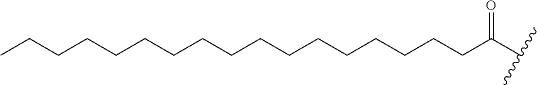 [SMILES: *C(=O)CCCCCCCCCCCCCCCCC] |
| 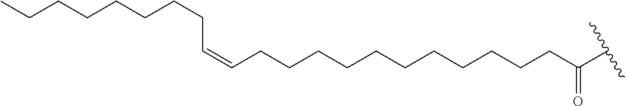 [SMILES: *C(=O)CCCCCCCCCCC/C=C\CCCCCCCC] | 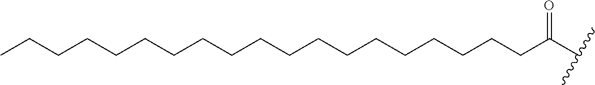 [SMILES: *C(=O)CCCCCCCCCCCCCCCCCCC] |
| 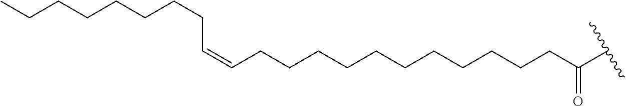 [SMILES: *C(=O)CCCCCCCCCCC/C=C\CCCCCCCC] | 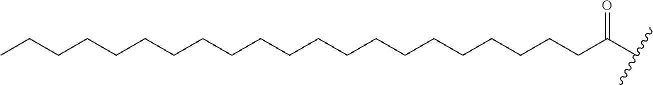 [SMILES: *C(=O)CCCCCCCCCCCCCCCCCCCCC] |
| 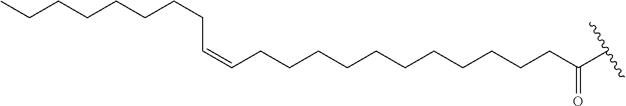 [SMILES: *C(=O)CCCCCCCCCCC/C=C\CCCCCCCC] | 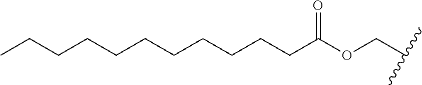 [SMILES: *COC(=O)CCCCCCCCCCC] |
| 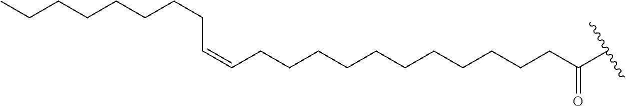 [SMILES: *C(=O)CCCCCCCCCCC/C=C\CCCCCCCC] | 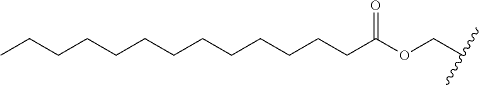 [SMILES: *COC(=O)CCCCCCCCCCCCC] |
| 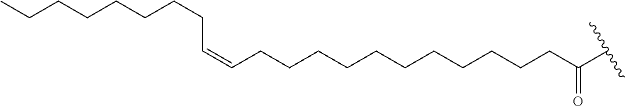 [SMILES: *C(=O)CCCCCCCCCCC/C=C\CCCCCCCC] | 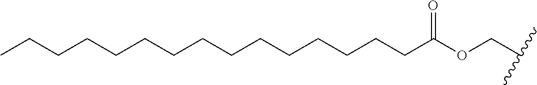 [SMILES: *COC(=O)CCCCCCCCCCCCCCC] |
| 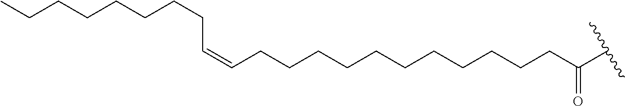 [SMILES: *C(=O)CCCCCCCCCCC/C=C\CCCCCCCC] | 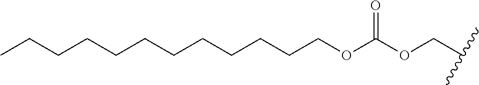 [SMILES: *COC(=O)OCCCCCCCCCCCC] |
| 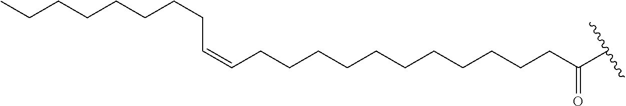 [SMILES: *C(=O)CCCCCCCCCCC/C=C\CCCCCCCC] | 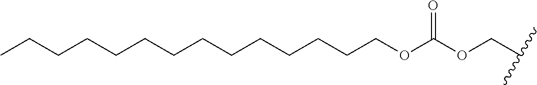 [SMILES: *COC(=O)OCCCCCCCCCCCCCC] |
| 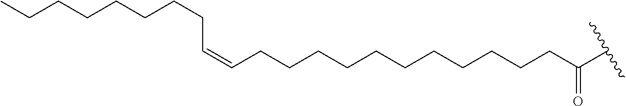 [SMILES: *C(=O)CCCCCCCCCCC/C=C\CCCCCCCC] | 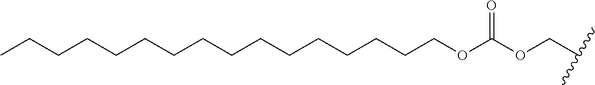 [SMILES: *COC(=O)OCCCCCCCCCCCCCCCC] |
| 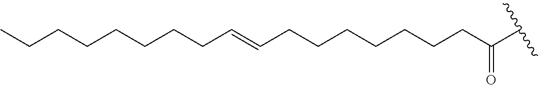 [SMILES: *C(=O)CCCCCCC/C=C/CCCCCCCC] | 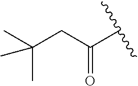 [SMILES: *C(=O)CC(C)(C)C] |
| 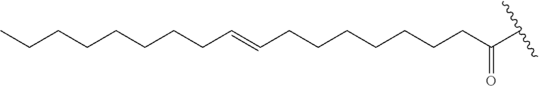 [SMILES: *C(=O)CCCCCCC/C=C/CCCCCCCC] | 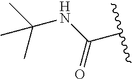 [SMILES: *C(=O)NC(C)(C)C] |
| 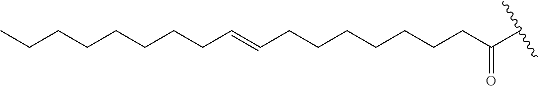 [SMILES: *C(=O)CCCCCCC/C=C/CCCCCCCC] | 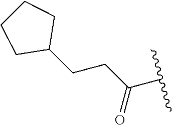 [SMILES: *C(=O)CCC1CCCC1] |
| 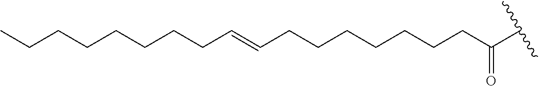 [SMILES: *C(=O)CCCCCCC/C=C/CCCCCCCC] | 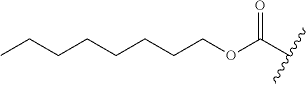 [SMILES: *C(=O)OCCCCCCCC] |
| 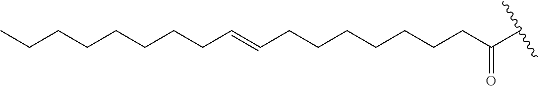 [SMILES: *C(=O)CCCCCCC/C=C/CCCCCCCC] | 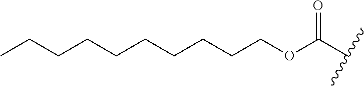 [SMILES: *C(=O)OCCCCCCCCCC] |
| 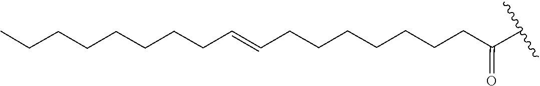 [SMILES: *C(=O)CCCCCCC/C=C/CCCCCCCC] | 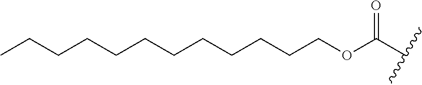 [SMILES: *C(=O)OCCCCCCCCCCCC] |
| 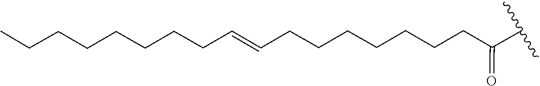 [SMILES: *C(=O)CCCCCCC/C=C/CCCCCCCC] | 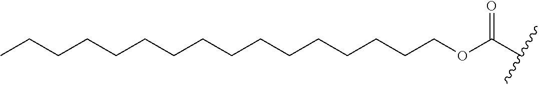 [SMILES: *C(=O)OCCCCCCCCCCCCCCCC] |
| 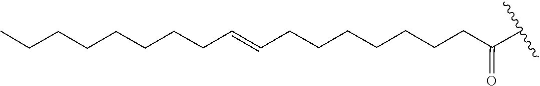 [SMILES: *C(=O)CCCCCCC/C=C/CCCCCCCC] | 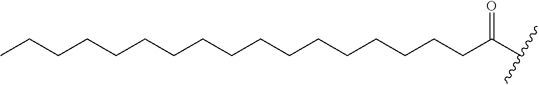 [SMILES: *C(=O)CCCCCCCCCCCCCCCCC] |
| 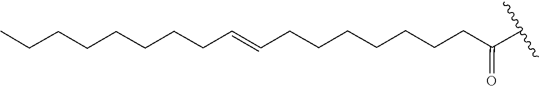 [SMILES: *C(=O)CCCCCCC/C=C/CCCCCCCC] | 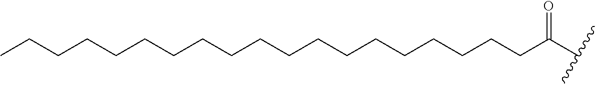 [SMILES: *C(=O)CCCCCCCCCCCCCCCCCCC] |
| 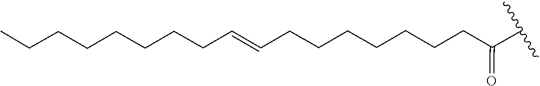 [SMILES: *C(=O)CCCCCCC/C=C/CCCCCCCC] | 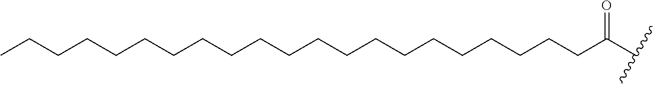 [SMILES: *C(=O)CCCCCCCCCCCCCCCCCCCCC] |
| 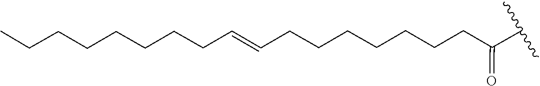 [SMILES: *C(=O)CCCCCCC/C=C/CCCCCCCC] | 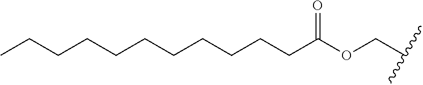 [SMILES: *COC(=O)CCCCCCCCCCC] |
| 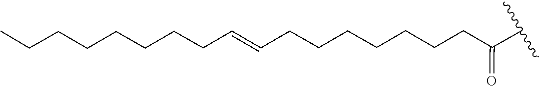 [SMILES: *C(=O)CCCCCCC/C=C/CCCCCCCC] | 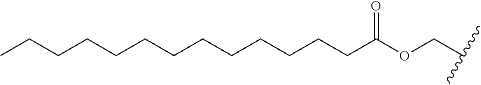 [SMILES: *COC(=O)CCCCCCCCCCCCC] |
| 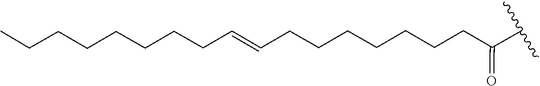 [SMILES: *C(=O)CCCCCCC/C=C/CCCCCCCC] | 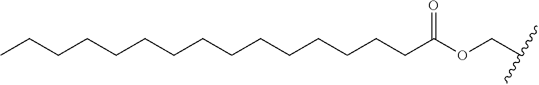 [SMILES: *COC(=O)CCCCCCCCCCCCCCC] |
| 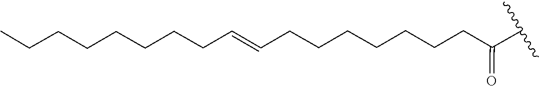 [SMILES: *C(=O)CCCCCCC/C=C/CCCCCCCC] | 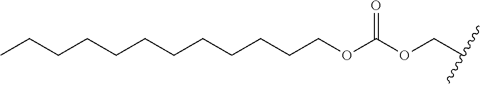 [SMILES: *COC(=O)OCCCCCCCCCCCC] |
| 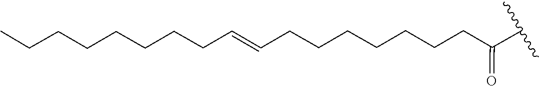 [SMILES: *C(=O)CCCCCCC/C=C/CCCCCCCC] | 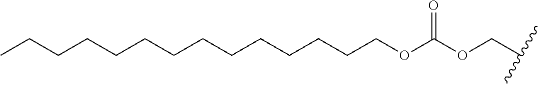 [SMILES: *COC(=O)OCCCCCCCCCCCCCC] |
| 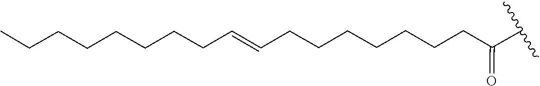 [SMILES: *C(=O)CCCCCCC/C=C/CCCCCCCC] | 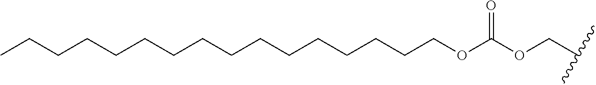 [SMILES: *COC(=O)OCCCCCCCCCCCCCCCC] |
| 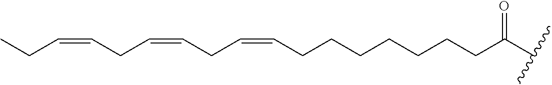 [SMILES: *C(=O)CCCCCCC/C=C\C/C=C\C/C=C\CC] | 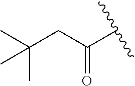 [SMILES: *C(=O)CC(C)(C)C] |
| 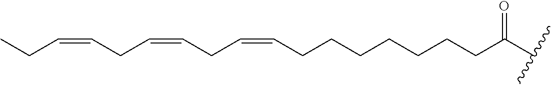 [SMILES: *C(=O)CCCCCCC/C=C\C/C=C\C/C=C\CC] | 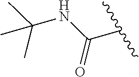 [SMILES: *C(=O)NC(C)(C)C] |
| 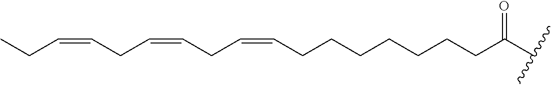 [SMILES: *C(=O)CCCCCCC/C=C\C/C=C\C/C=C\CC] | 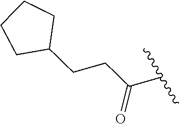 [SMILES: *C(=O)CCC1CCCC1] |
| 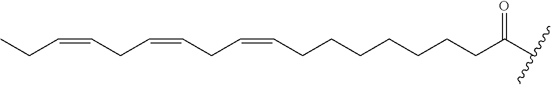 [SMILES: *C(=O)CCCCCCC/C=C\C/C=C\C/C=C\CC] | 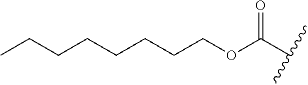 [SMILES: *C(=O)OCCCCCCCC] |
| 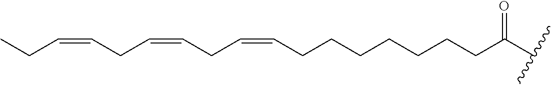 [SMILES: *C(=O)CCCCCCC/C=C\C/C=C\C/C=C\CC] | 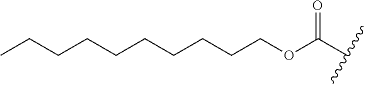 [SMILES: *C(=O)OCCCCCCCCCC] |
| 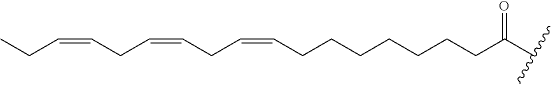 [SMILES: *C(=O)CCCCCCC/C=C\C/C=C\C/C=C\CC] | 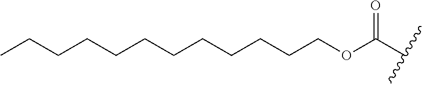 [SMILES: *C(=O)OCCCCCCCCCCCC] |
| 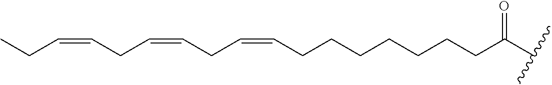 [SMILES: *C(=O)CCCCCCC/C=C\C/C=C\C/C=C\CC] | 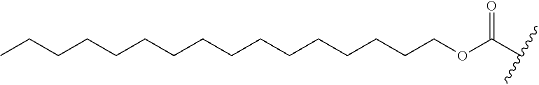 [SMILES: *C(=O)OCCCCCCCCCCCCCCCC] |
| 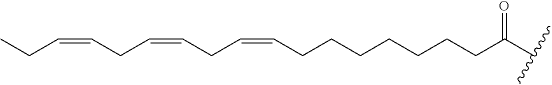 [SMILES: *C(=O)CCCCCCC/C=C\C/C=C\C/C=C\CC] | 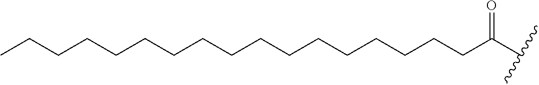 [SMILES: *C(=O)CCCCCCCCCCCCCCCCC] |
| 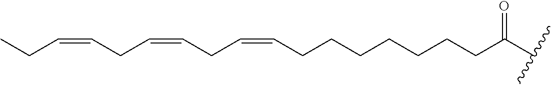 [SMILES: *C(=O)CCCCCCC/C=C\C/C=C\C/C=C\CC] | 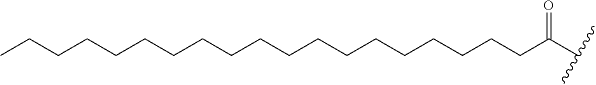 [SMILES: *C(=O)CCCCCCCCCCCCCCCCCCC] |
| 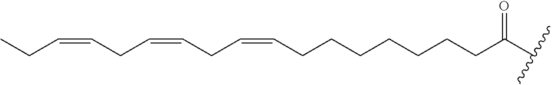 [SMILES: *C(=O)CCCCCCC/C=C\C/C=C\C/C=C\CC] | 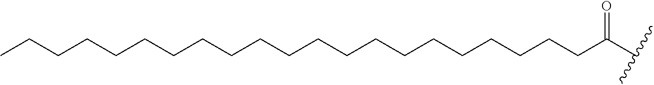 [SMILES: *C(=O)CCCCCCCCCCCCCCCCCCCCC] |
| 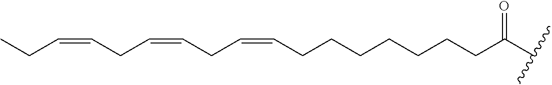 [SMILES: *C(=O)CCCCCCC/C=C\C/C=C\C/C=C\CC] | 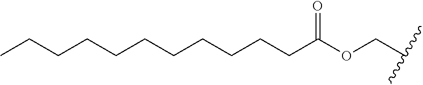 [SMILES: *COC(=O)CCCCCCCCCCC] |
| 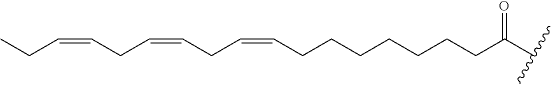 [SMILES: *C(=O)CCCCCCC/C=C\C/C=C\C/C=C\CC] | 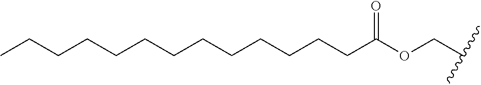 [SMILES: *COC(=O)CCCCCCCCCCCCC] |
| 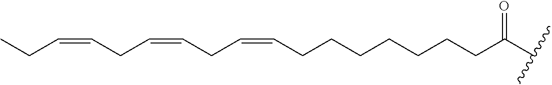 [SMILES: *C(=O)CCCCCCC/C=C\C/C=C\C/C=C\CC] | 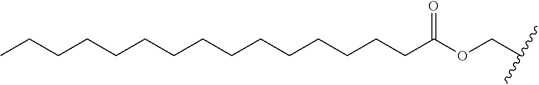 [SMILES: *COC(=O)CCCCCCCCCCCCCCC] |
| 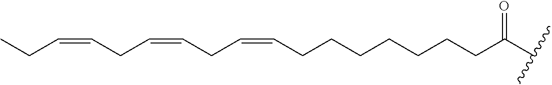 [SMILES: *C(=O)CCCCCCC/C=C\C/C=C\C/C=C\CC] | 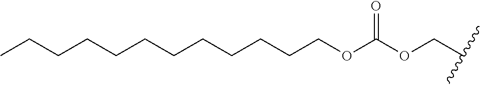 [SMILES: *COC(=O)OCCCCCCCCCCCC] |
| 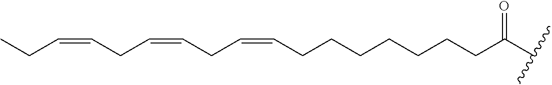 [SMILES: *C(=O)CCCCCCC/C=C\C/C=C\C/C=C\CC] | 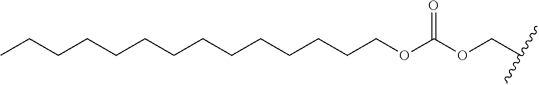 [SMILES: *COC(=O)OCCCCCCCCCCCCCC] |
| 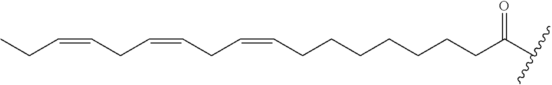 [SMILES: *C(=O)CCCCCCC/C=C\C/C=C\C/C=C\CC] | 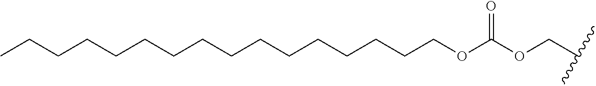 [SMILES: *COC(=O)OCCCCCCCCCCCCCCCC] |
| tBu | 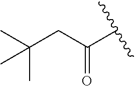 [SMILES: *C(=O)CC(C)(C)C] |
| tBu | 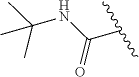 [SMILES: *C(=O)NC(C)(C)C] |
| tBu | 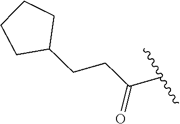 [SMILES: *C(=O)CCC1CCCC1] |
| tBu | 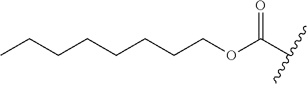 [SMILES: *C(=O)OCCCCCCCC] |
| tBu | 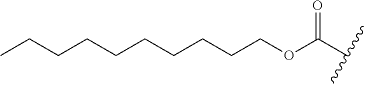 [SMILES: *C(=O)OCCCCCCCCCC] |
| tBu | 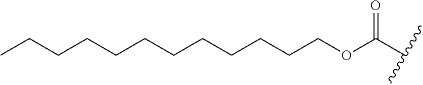 [SMILES: *C(=O)OCCCCCCCCCCCC] |
| tBu | 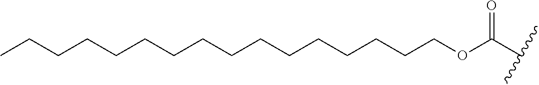 [SMILES: *C(=O)OCCCCCCCCCCCCCCCC] |
| tBu | 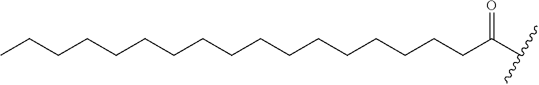 [SMILES: *C(=O)CCCCCCCCCCCCCCCCC] |
| tBu | 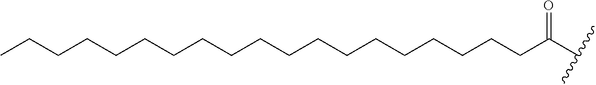 [SMILES: *C(=O)CCCCCCCCCCCCCCCCCCC] |
| tBu | 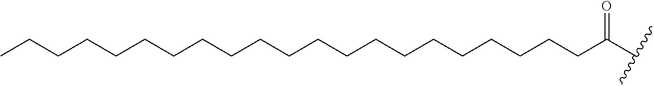 [SMILES: *C(=O)CCCCCCCCCCCCCCCCCCCCC] |
| tBu | 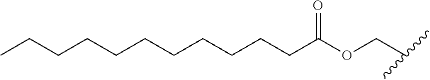 [SMILES: *COC(=O)CCCCCCCCCCC] |
| tBu | 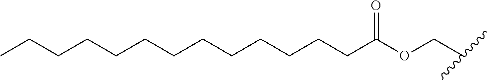 [SMILES: *COC(=O)CCCCCCCCCCCCC] |
| tBu | 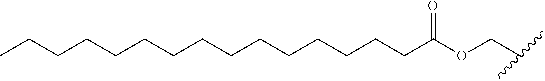 [SMILES: *COC(=O)CCCCCCCCCCCCCCC] |
| tBu | 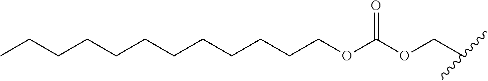 [SMILES: *COC(=O)OCCCCCCCCCCCC] |
| tBu | 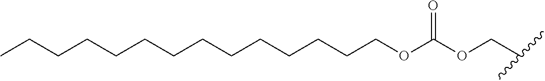 [SMILES: *COC(=O)OCCCCCCCCCCCCCC] |
| tBu | 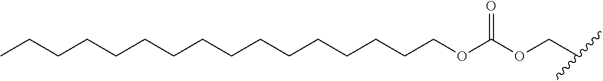 [SMILES: *COC(=O)OCCCCCCCCCCCCCCCC] |
| phenyl | 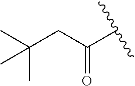 [SMILES: *C(=O)CC(C)(C)C] |
| phenyl | 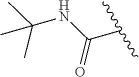 [SMILES: *C(=O)NC(C)(C)C] |
| phenyl | 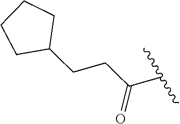 [SMILES: *C(=O)CCC1CCCC1] |
| phenyl | 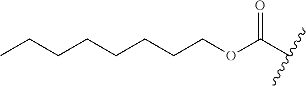 [SMILES: *C(=O)OCCCCCCCC] |
| phenyl | 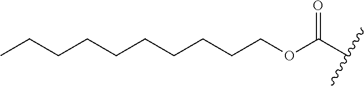 [SMILES: *C(=O)OCCCCCCCCCC] |
| phenyl | 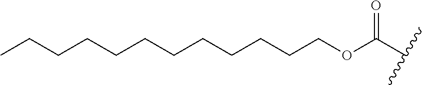 [SMILES: *C(=O)OCCCCCCCCCCCC] |
| phenyl | 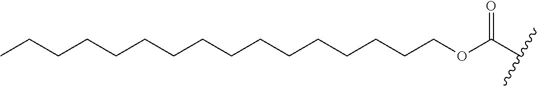 [SMILES: *C(=O)OCCCCCCCCCCCCCCCC] |
| phenyl | 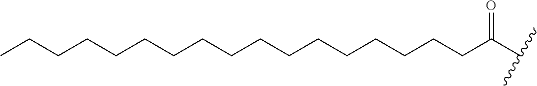 [SMILES: *C(=O)CCCCCCCCCCCCCCCCC] |
| phenyl | 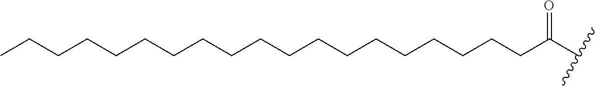 [SMILES: *C(=O)CCCCCCCCCCCCCCCCCCC] |
| phenyl | 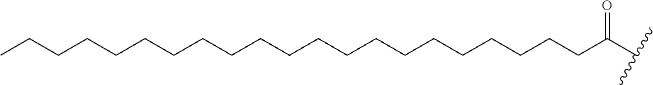 [SMILES: *C(=O)CCCCCCCCCCCCCCCCCCCCC] |
| phenyl | 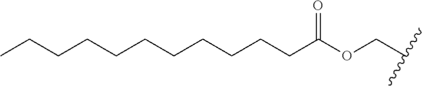 [SMILES: *COC(=O)CCCCCCCCCCC] |
| phenyl | 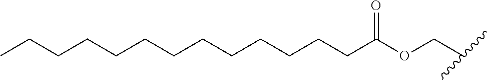 [SMILES: *COC(=O)CCCCCCCCCCCCC] |
| phenyl | 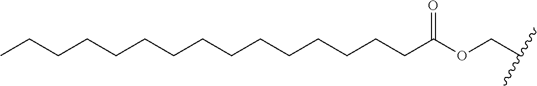 [SMILES: *COC(=O)CCCCCCCCCCCCCCC] |
| phenyl | 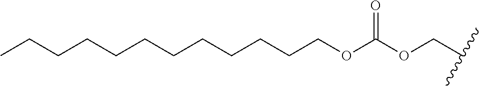 [SMILES: *COC(=O)OCCCCCCCCCCCC] |
| phenyl | 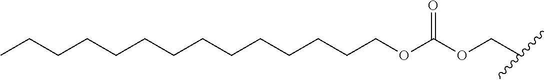 [SMILES: *COC(=O)OCCCCCCCCCCCCCC] |
| phenyl | 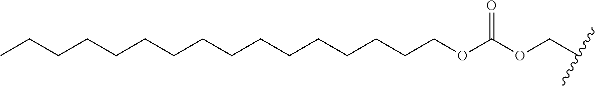 [SMILES: *COC(=O)OCCCCCCCCCCCCCCCC] |
| 4-chlorophenyl | 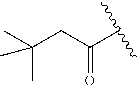 [SMILES: *C(=O)CC(C)(C)C] |
| 4-chlorophenyl | 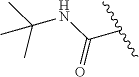 [SMILES: *C(=O)NC(C)(C)C] |
| 4-chlorophenyl | 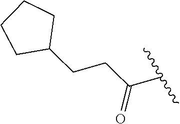 [SMILES: *C(=O)CCC1CCCC1] |
| 4-chlorophenyl | 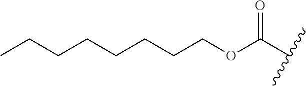 [SMILES: *C(=O)OCCCCCCCC] |
| 4-chlorophenyl | 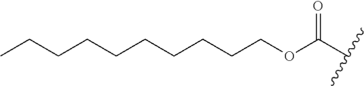 [SMILES: *C(=O)OCCCCCCCCCC] |
| 4-chlorophenyl | 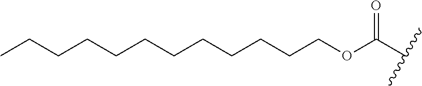 [SMILES: *C(=O)OCCCCCCCCCCCC] |
| 4-chlorophenyl | 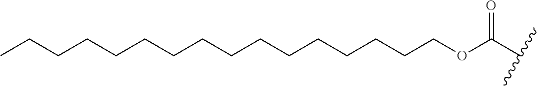 [SMILES: *C(=O)OCCCCCCCCCCCCCCCC] |
| 4-chlorophenyl | 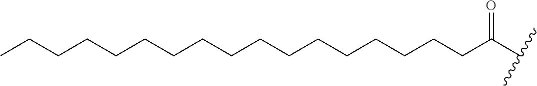 [SMILES: *C(=O)CCCCCCCCCCCCCCCCC] |
| 4-chlorophenyl | 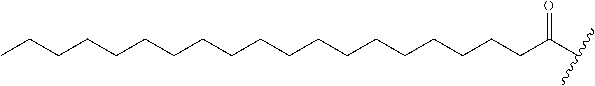 [SMILES: *C(=O)CCCCCCCCCCCCCCCCCCC] |
| 4-chlorophenyl | 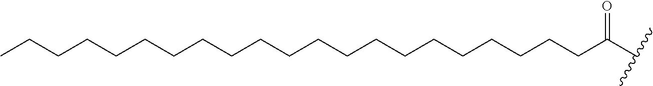 [SMILES: *C(=O)CCCCCCCCCCCCCCCCCCCCC] |
| 4-chlorophenyl | 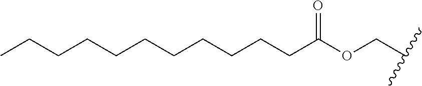 [SMILES: *COC(=O)CCCCCCCCCCC] |
| 4-chlorophenyl | 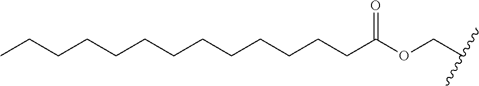 [SMILES: *COC(=O)CCCCCCCCCCCCC] |
| 4-chlorophenyl | 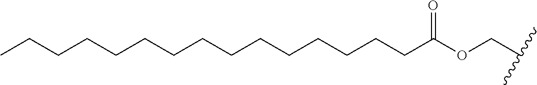 [SMILES: *COC(=O)CCCCCCCCCCCCCCC] |
| 4-chlorophenyl | 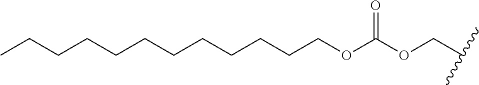 [SMILES: *COC(=O)OCCCCCCCCCCCC] |
| 4-chlorophenyl | 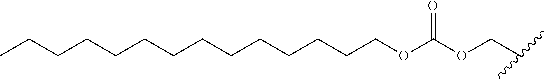 [SMILES: *COC(=O)OCCCCCCCCCCCCCC] |
| 4-chlorophenyl | 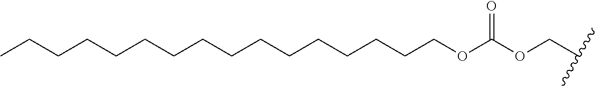 [SMILES: *COC(=O)OCCCCCCCCCCCCCCCC] |
| 3,4-dichlorophenyl | 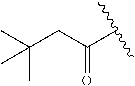 [SMILES: *C(=O)CC(C)(C)C] |
| 3,4-dichlorophenyl | 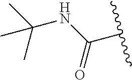 [SMILES: *C(=O)NC(C)(C)C] |
| 3,4-dichlorophenyl | 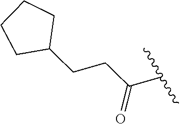 [SMILES: *C(=O)CCC1CCCC1] |
| 3,4-dichlorophenyl | 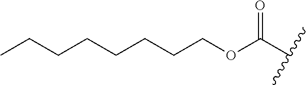 [SMILES: *C(=O)OCCCCCCCC] |
| 3,4-dichlorophenyl | 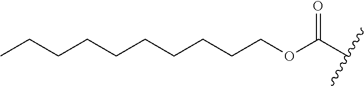 [SMILES: *C(=O)OCCCCCCCCCC] |
| 3,4-dichlorophenyl | 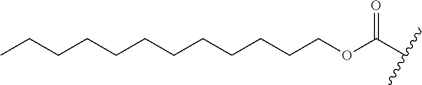 [SMILES: *C(=O)OCCCCCCCCCCCC] |
| 3,4-dichlorophenyl | 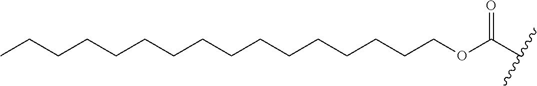 [SMILES: *C(=O)OCCCCCCCCCCCCCCCC] |
| 3,4-dichlorophenyl | 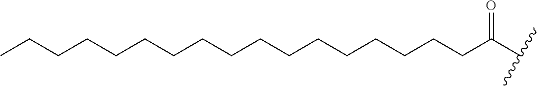 [SMILES: *C(=O)CCCCCCCCCCCCCCCCC] |
| 3,4-dichlorophenyl | 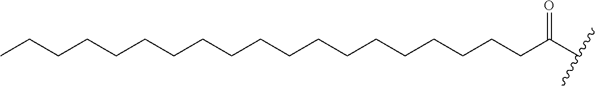 [SMILES: *C(=O)CCCCCCCCCCCCCCCCCCC] |
| 3,4-dichlorophenyl | 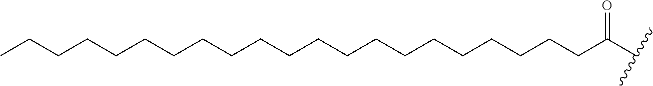 [SMILES: *C(=O)CCCCCCCCCCCCCCCCCCCCC] |
| 3,4-dichlorophenyl | 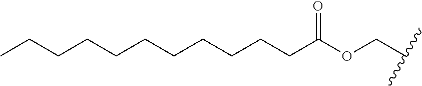 [SMILES: *COC(=O)CCCCCCCCCCC] |
| 3,4-dichlorophenyl | 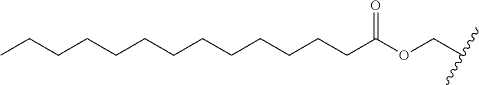 [SMILES: *COC(=O)CCCCCCCCCCCCC] |
| 3,4-dichlorophenyl | 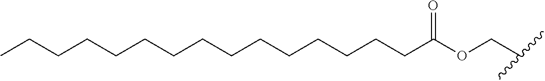 [SMILES: *COC(=O)CCCCCCCCCCCCCCC] |
| 3,4-dichlorophenyl | 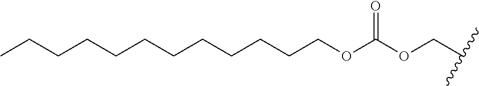 [SMILES: *COC(=O)OCCCCCCCCCCCC] |
| 3,4-dichlorophenyl | 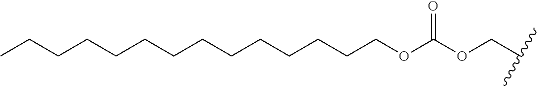 [SMILES: *COC(=O)OCCCCCCCCCCCCCC] |
| 3,4-dichlorophenyl | 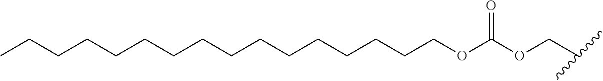 [SMILES: *COC(=O)OCCCCCCCCCCCCCCCC] |
| 4-methylphenyl | 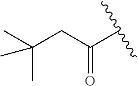 [SMILES: *C(=O)CC(C)(C)C] |
| 4-methylphenyl | 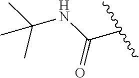 [SMILES: *C(=O)NC(C)(C)C] |
| 4-methylphenyl | 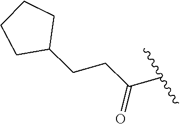 [SMILES: *C(=O)CCC1CCCC1] |
| 4-methylphenyl | 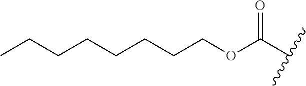 [SMILES: *C(=O)OCCCCCCCC] |
| 4-methylphenyl | 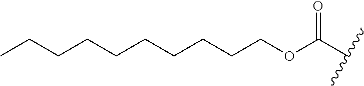 [SMILES: *C(=O)OCCCCCCCCCC] |
| 4-methylphenyl | 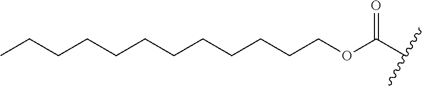 [SMILES: *C(=O)OCCCCCCCCCCCC] |
| 4-methylphenyl | 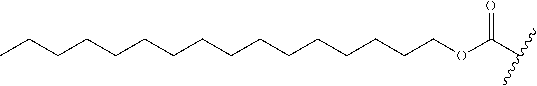 [SMILES: *C(=O)OCCCCCCCCCCCCCCCC] |
| 4-methylphenyl | 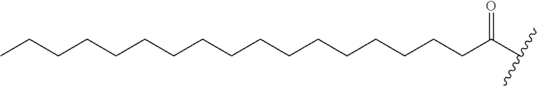 [SMILES: *C(=O)CCCCCCCCCCCCCCCCC] |
| 4-methylphenyl | 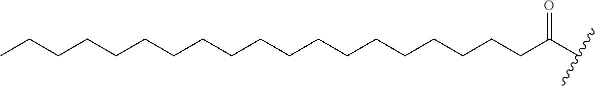 [SMILES: *C(=O)CCCCCCCCCCCCCCCCCCC] |
| 4-methylphenyl | 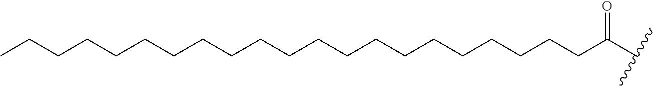 [SMILES: *C(=O)CCCCCCCCCCCCCCCCCCCCC] |
| 4-methylphenyl | 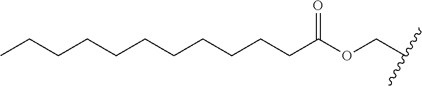 [SMILES: *COC(=O)CCCCCCCCCCC] |
| 4-methylphenyl | 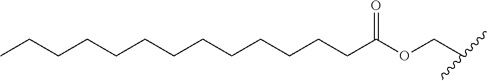 [SMILES: *COC(=O)CCCCCCCCCCCCC] |
| 4-methylphenyl | 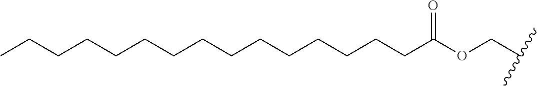 [SMILES: *COC(=O)CCCCCCCCCCCCCCC] |
| 4-methylphenyl | 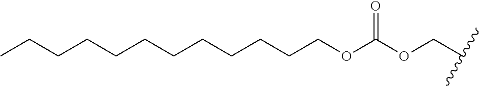 [SMILES: *COC(=O)OCCCCCCCCCCCC] |
| 4-methylphenyl | 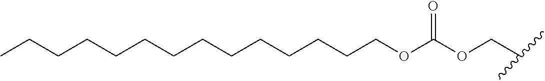 [SMILES: *COC(=O)OCCCCCCCCCCCCCC] |
| 4-methylphenyl | 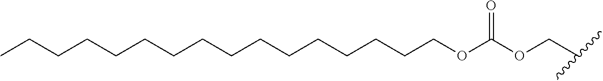 [SMILES: *COC(=O)OCCCCCCCCCCCCCCCC] |
| 4-methoxyphenyl | 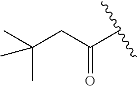 [SMILES: *C(=O)CC(C)(C)C] |
| 4-methoxyphenyl | 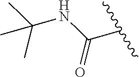 [SMILES: *C(=O)NC(C)(C)C] |
| 4-methoxyphenyl | 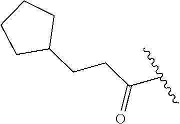 [SMILES: *C(=O)CCC1CCCC1] |
| 4-methoxyphenyl | 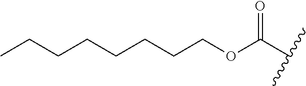 [SMILES: *C(=O)OCCCCCCCC] |
| 4-methoxyphenyl | 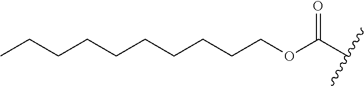 [SMILES: *C(=O)OCCCCCCCCCC] |
| 4-methoxyphenyl | 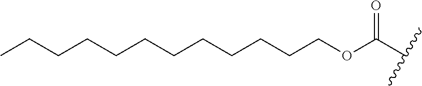 [SMILES: *C(=O)OCCCCCCCCCCCC] |
| 4-methoxyphenyl | 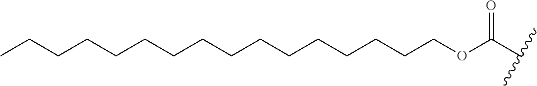 [SMILES: *C(=O)OCCCCCCCCCCCCCCCC] |
| 4-methoxyphenyl | 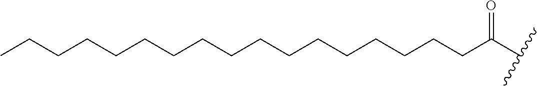 [SMILES: *C(=O)CCCCCCCCCCCCCCCCC] |
| 4-methoxyphenyl | 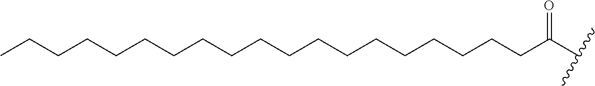 [SMILES: *C(=O)CCCCCCCCCCCCCCCCCCC] |
| 4-methoxyphenyl | 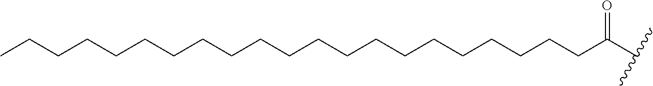 [SMILES: *C(=O)CCCCCCCCCCCCCCCCCCCCC] |
| 4-methoxyphenyl | 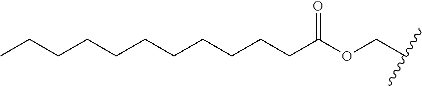 [SMILES: *COC(=O)CCCCCCCCCCC] |
| 4-methoxyphenyl | 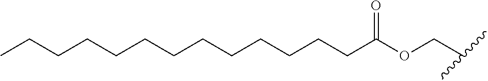 [SMILES: *COC(=O)CCCCCCCCCCCCC] |
| 4-methoxyphenyl | 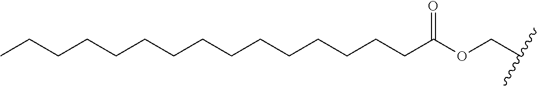 [SMILES: *COC(=O)CCCCCCCCCCCCCCC] |
| 4-methoxyphenyl | 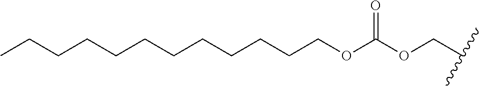 [SMILES: *COC(=O)OCCCCCCCCCCCC] |
| 4-methoxyphenyl | 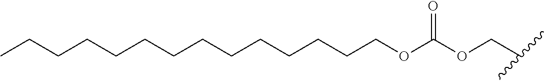 [SMILES: *COC(=O)OCCCCCCCCCCCCCC] |
| 4-methoxyphenyl | 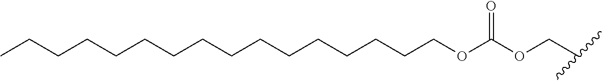 [SMILES: *COC(=O)OCCCCCCCCCCCCCCCC] |

In a 58^{th} to aspect provided herein is a compound, or pharmaceutically acceptable salt thereof, having a structure provided in Formula (I-19),

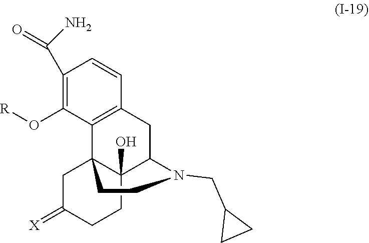

wherein X is ═O and R is R⁴OP(OH)(O)—O—CH(R³)—, R³ and R⁴ are any of the pair of R³/R⁴ groups recited in Table 9. Alternatively, X is ═CH₂, R is R⁴OP(OH)(O)—O—CH(R³)— and R³ and R⁴ are any of the pair of R³/R⁴ groups recited in Table 9.

In a 59^{th} aspect, provided herein is a compound, or pharmaceutically acceptable salt thereof, having a structure provided in Formula (II-19),

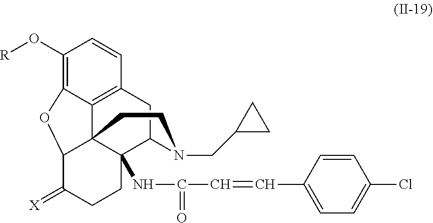

wherein X is ═O, R is R⁴OP(OH)(O)—O—CH(R³)— and R³ and R⁴ are any of the pair of R³/R⁴ groups recited in Table 9. Alternatively, X is ═CH₂, R is R⁴OP(OH)(O)—O—CH(R³)— and R³ and R⁴ are any of the pair of R³/R⁴ groups recited in Table 9.

In a 60^{th} aspect, provided herein is a compound, or pharmaceutically acceptable salt thereof, having a structure provided in Formula (III-19),

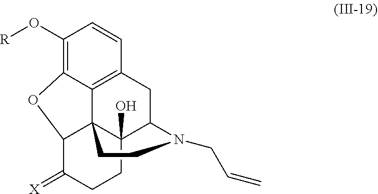

wherein X is ═O, R is R⁴OP(OH)(O)—O—CH(R³)— and R³ and R⁴ are any of the pair of R³/R⁴ groups recited in Table 9. Alternatively, X is ═CH₂, R is
R⁴OP(OH)(O)—O—CH(R³)— and R³ and R⁴ are any of the pair of R³/R⁴ groups recited in Table 9.

TABLE 9

| R³ | R⁴ |
|---|---|
| H | Me |
| H | —C₇H₁₅ |
| H | —C₉H₁₉ |
| H | —C₁₁H₂₃ |
| H | —C₁₃H₂₇ |
| H | —C₁₅H₃₁ |
| H | —C₁₇H₃₅ |
| H | 2816 |
| H | 2817 |
| H | 2818 |
| H | 2819 |
| H | 2820 |
| H | tBu |
| H | phenyl |
| H | 4-chlorophenyl |
| H | 3,4-dichlorophenyl |
| H | 4-methylphenyl |
| H | 4-methoxyphenyl |
| Me | Me |
| Me | —C₇H₁₅ |
| Me | —C₉H₁₉ |
| Me | —C₁₁H₂₃ |
| Me | —C₁₃H₂₇ |
| Me | —C₁₅H₃₁ |
| Me | —C₁₇H₃₅ |
| Me | 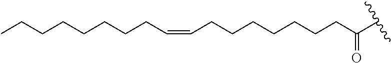 [SMILES: *C(=O)CCCCCCC/C=C\CCCCCCCC] |
| Me | 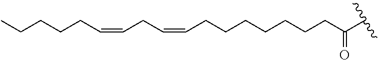 [SMILES: *C(=O)CCCCCCC/C=C\C/C=C\CCCCC] |
| Me | 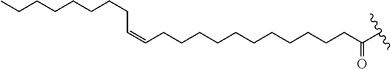 [SMILES: *C(=O)CCCCCCCCCCC/C=C\CCCCCCCC] |
| Me | 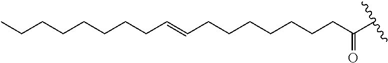 [SMILES: *C(=O)CCCCCCC/C=C/CCCCCCCC] |
| Me | 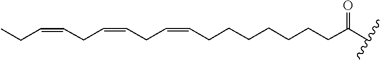 [SMILES: *C(=O)CCCCCCC/C=C\C/C=C\C/C=C\CC] |
| Me | tBu |
| Me | phenyl |
| Me | 4-chlorophenyl |
| Me | 3,4-dichlorophenyl |
| Me | 4-methylphenyl |
| Me | 4-methoxyphenyl |
| tBu | Me |
| tBu | —C₇H₁₅ |
| tBu | —C₉H₁₉ |
| tBu | —C₁₁H₂₃ |
| tBu | —C₁₃H₂₇ |
| tBu | —C₁₅H₃₁ |
| tBu | —C₁₇H₃₅ |
| tBu | 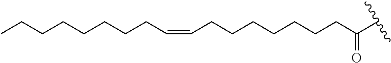 [SMILES: *C(=O)CCCCCCC/C=C\CCCCCCCC] |
| tBu | 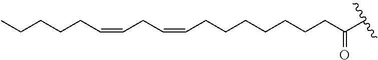 [SMILES: *C(=O)CCCCCCC/C=C\C/C=C\CCCCC] |
| tBu | 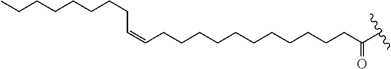 [SMILES: *C(=O)CCCCCCCCCCC/C=C\CCCCCCCC] |
| tBu | 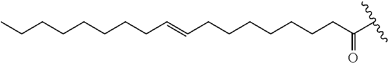 [SMILES: *C(=O)CCCCCCC/C=C/CCCCCCCC] |
| tBu | 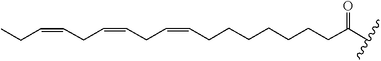 [SMILES: *C(=O)CCCCCCC/C=C\C/C=C\C/C=C\CC] |
| tBu | phenyl |
| tBu | tBu |
| tBu | 4-chlorophenyl |
| tBu | 3,4-dichlorophenyl |
| tBu | 4-methylphenyl |
| tBu | 4-methoxyphenyl |
| —C₇H₁₅ | Me |
| —C₇H₁₅ | —C₇H₁₅ |
| —C₇H₁₅ | —C₉H₁₉ |
| —C₇H₁₅ | —C₁₁H₂₃ |
| —C₇H₁₅ | —C₁₃H₂₇ |
| —C₇H₁₅ | —C₁₅H₃₁ |
| —C₇H₁₅ | —C₁₇H₃₅ |
| —C₇H₁₅ | 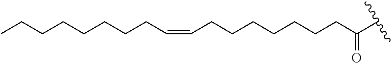 [SMILES: *C(=O)CCCCCCC/C=C\CCCCCCCC] |
| —C₇H₁₅ | 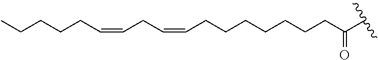 [SMILES: *C(=O)CCCCCCC/C=C\C/C=C\CCCCC] |
| —C₇H₁₅ | 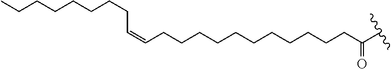 [SMILES: *C(=O)CCCCCCCCCCC/C=C\CCCCCCCC] |
| —C₇H₁₅ | 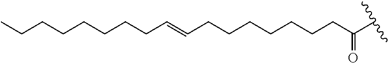 [SMILES: *C(=O)CCCCCCC/C=C/CCCCCCCC] |
| —C₇H₁₅ | 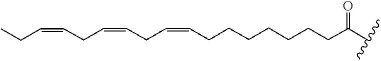 [SMILES: *C(=O)CCCCCCC/C=C\C/C=C\C/C=C\CC] |
| —C₇H₁₅ | tBu |
| —C₇H₁₅ | phenyl |
| —C₇H₁₅ | 4-chlorophenyl |
| —C₇H₁₅ | 3,4-dichlorophenyl |
| —C₇H₁₅ | 4-methylphenyl |
| —C₇H₁₅ | 4-methoxyphenyl |
| —C₉H₁₉ | Me |
| —C₉H₁₉ | —C₇H₁₅ |
| —C₉H₁₉ | —C₉H₁₉ |
| —C₉H₁₉ | —C₁₁H₂₃ |
| —C₉H₁₉ | —C₁₃H₂₇ |
| —C₉H₁₉ | —C₁₅H₃₁ |
| —C₉H₁₉ | —C₁₇H₃₅ |
| —C₉H₁₉ | 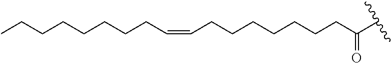 [SMILES: *C(=O)CCCCCCC/C=C\CCCCCCCC] |
| —C₉H₁₉ | 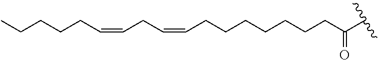 [SMILES: *C(=O)CCCCCCC/C=C\C/C=C\CCCCC] |
| —C₉H₁₉ | 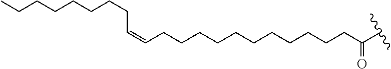 [SMILES: *C(=O)CCCCCCCCCCC/C=C\CCCCCCCC] |
| —C₉H₁₉ | 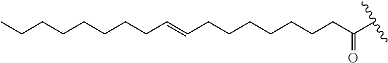 [SMILES: *C(=O)CCCCCCC/C=C/CCCCCCCC] |
| —C₉H₁₉ | 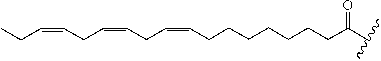 [SMILES: *C(=O)CCCCCCC/C=C\C/C=C\C/C=C\CC] |
| —C₉H₁₉ | tBu |
| —C₉H₁₉ | phenyl |
| —C₉H₁₉ | 4-chlorophenyl |
| —C₉H₁₉ | 3,4-dichlorophenyl |
| —C₉H₁₉ | 4-methylphenyl |
| —C₉H₁₉ | 4-methoxyphenyl |
| —C₁₁H₂₃ | Me |
| —C₁₁H₂₃ | —C₇H₁₅ |
| —C₁₁H₂₃ | —C₉H₁₉ |
| —C₁₁H₂₃ | —C₁₁H₂₃ |
| —C₁₁H₂₃ | —C₁₃H₂₇ |
| —C₁₁H₂₃ | —C₁₅H₃₁ |
| —C₁₁H₂₃ | —C₁₇H₃₅ |
| —C₁₁H₂₃ | 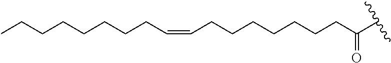 [SMILES: *C(=O)CCCCCCC/C=C\CCCCCCCC] |
| —C₁₁H₂₃ | 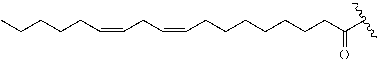 [SMILES: *C(=O)CCCCCCC/C=C\C/C=C\CCCCC] |
| —C₁₁H₂₃ | 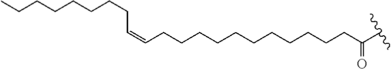 [SMILES: *C(=O)CCCCCCCCCCC/C=C\CCCCCCCC] |
| —C₁₁H₂₃ | 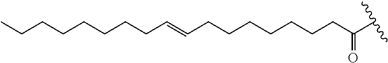 [SMILES: *C(=O)CCCCCCC/C=C/CCCCCCCC] |
| —C₁₁H₂₃ | 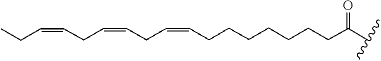 [SMILES: *C(=O)CCCCCCC/C=C\C/C=C\C/C=C\CC] |
| —C₁₁H₂₃ | tBu |
| —C₁₁H₂₃ | phenyl |
| —C₁₁H₂₃ | 4-chlorophenyl |
| —C₁₁H₂₃ | 3,4-dichlorophenyl |
| —C₁₁H₂₃ | 4-methylphenyl |
| —C₁₁H₂₃ | 4-methoxyphenyl |
| —C₁₃H₂₇ | Me |
| —C₁₃H₂₇ | —C₇H₁₅ |
| —C₁₃H₂₇ | —C₉H₁₉ |
| —C₁₃H₂₇ | —C₁₁H₂₃ |
| —C₁₃H₂₇ | —C₁₃H₂₇ |
| —C₁₃H₂₇ | —C₁₅H₃₁ |
| —C₁₃H₂₇ | —C₁₇H₃₅ |
| —C₁₃H₂₇ | 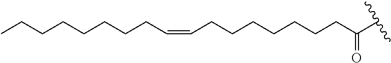 [SMILES: *C(=O)CCCCCCC/C=C\CCCCCCCC] |
| —C₁₃H₂₇ | 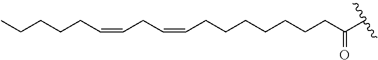 [SMILES: *C(=O)CCCCCCC/C=C\C/C=C\CCCCC] |
| —C₁₃H₂₇ | 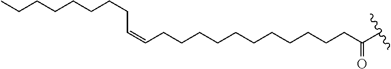 [SMILES: *C(=O)CCCCCCCCCCC/C=C\CCCCCCCC] |
| —C₁₃H₂₇ | 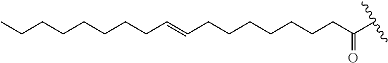 [SMILES: *C(=O)CCCCCCC/C=C/CCCCCCCC] |
| —C₁₃H₂₇ | 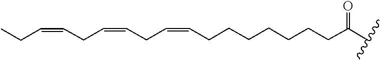 [SMILES: *C(=O)CCCCCCC/C=C\C/C=C\C/C=C\CC] |
| —C₁₃H₂₇ | tBu |
| —C₁₃H₂₇ | phenyl |
| —C₁₃H₂₇ | 4-chlorophenyl |
| —C₁₃H₂₇ | 3,4-dichlorophenyl |
| —C₁₃H₂₇ | 4-methylphenyl |
| —C₁₃H₂₇ | 4-methoxyphenyl |
| —C₁₅H₃₁ | Me |
| —C₁₅H₃₁ | —C₇H₁₅ |
| —C₁₅H₃₁ | —C₉H₁₉ |
| —C₁₅H₃₁ | —C₁₁H₂₃ |
| —C₁₅H₃₁ | —C₁₃H₂₇ |
| —C₁₅H₃₁ | —C₁₅H₃₁ |
| —C₁₅H₃₁ | —C₁₇H₃₅ |
| —C₁₅H₃₁ | 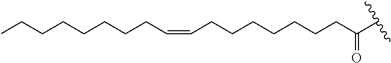 [SMILES: *C(=O)CCCCCCC/C=C\CCCCCCCC] |
| —C₁₅H₃₁ | 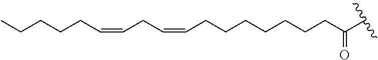 [SMILES: *C(=O)CCCCCCC/C=C\C/C=C\CCCCC] |
| —C₁₅H₃₁ | 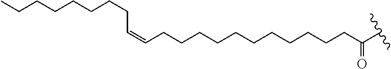 [SMILES: *C(=O)CCCCCCCCCCC/C=C\CCCCCCCC] |
| —C₁₅H₃₁ | 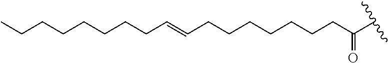 [SMILES: *C(=O)CCCCCCC/C=C/CCCCCCCC] |
| —C₁₅H₃₁ | 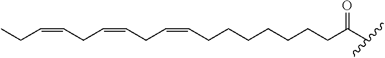 [SMILES: *C(=O)CCCCCCC/C=C\C/C=C\C/C=C\CC] |
| —C₁₅H₃₁ | tBu |
| —C₁₅H₃₁ | phenyl |
| —C₁₅H₃₁ | 4-chlorophenyl |
| —C₁₅H₃₁ | 3,4-dichlorophenyl |
| —C₁₅H₃₁ | 4-methylphenyl |
| —C₁₅H₃₁ | 4-methoxyphenyl |
| —C₁₇H₃₅ | Me |
| —C₁₇H₃₅ | —C₇H₁₅ |
| —C₁₇H₃₅ | —C₉H₁₉ |
| —C₁₇H₃₅ | —C₁₁H₂₃ |
| —C₁₇H₃₅ | —C₁₃H₂₇ |
| —C₁₇H₃₅ | —C₁₅H₃₁ |
| —C₁₇H₃₅ | —C₁₇H₃₅ |
| —C₁₇H₃₅ | 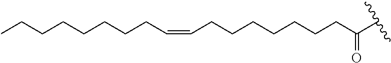 [SMILES: *C(=O)CCCCCCC/C=C\CCCCCCCC] |
| —C₁₇H₃₅ | 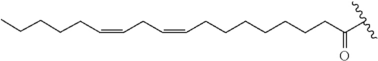 [SMILES: *C(=O)CCCCCCC/C=C\C/C=C\CCCCC] |
| —C₁₇H₃₅ | 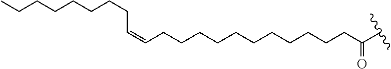 [SMILES: *C(=O)CCCCCCCCCCC/C=C\CCCCCCCC] |
| —C₁₇H₃₅ | 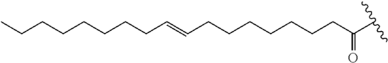 [SMILES: *C(=O)CCCCCCC/C=C/CCCCCCCC] |
| —C₁₇H₃₅ | 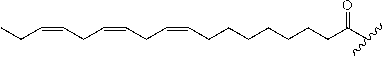 [SMILES: *C(=O)CCCCCCC/C=C\C/C=C\C/C=C\CC] |
| —C₁₇H₃₅ | tBu |
| —C₁₇H₃₅ | phenyl |
| —C₁₇H₃₅ | 4-chlorophenyl |
| —C₁₇H₃₅ | 3,4-dichlorophenyl |
| —C₁₇H₃₅ | 4-methylphenyl |
| —C₁₇H₃₅ | 4-methoxyphenyl |
| Phenyl | Me |
| Phenyl | —C₇H₁₅ |
| Phenyl | —C₉H₁₉ |
| Phenyl | —C₁₁H₂₃ |
| Phenyl | —C₁₃H₂₇ |
| Phenyl | —C₁₅H₃₁ |
| Phenyl | —C₁₇H₃₅ |
| Phenyl | 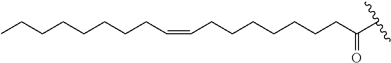 [SMILES: *C(=O)CCCCCCC/C=C\CCCCCCCC] |
| Phenyl | 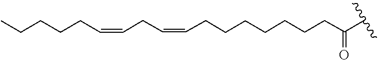 [SMILES: *C(=O)CCCCCCC/C=C\C/C=C\CCCCC] |
| Phenyl | 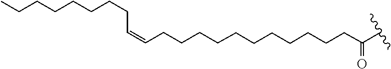 [SMILES: *C(=O)CCCCCCCCCCC/C=C\CCCCCCCC] |
| Phenyl | 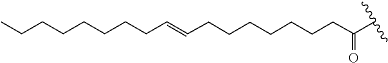 [SMILES: *C(=O)CCCCCCC/C=C/CCCCCCCC] |
| Phenyl | 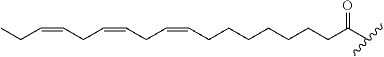 [SMILES: *C(=O)CCCCCCC/C=C\C/C=C\C/C=C\CC] |
| Phenyl | tBu |
| Phenyl | phenyl |
| Phenyl | 4-chlorophenyl |
| Phenyl | 3,4-dichlorophenyl |
| Phenyl | 4-methylphenyl |
| Phenyl | 4-methoxyphenyl |
| 4-chlorophenyl | Me |
| 4-chlorophenyl | —C₇H₁₅ |
| 4-chlorophenyl | —C₉H₁₉ |
| 4-chlorophenyl | —C₁₁H₂₃ |
| 4-chlorophenyl | —C₁₃H₂₇ |
| 4-chlorophenyl | —C₁₅H₃₁ |
| 4-chlorophenyl | —C₁₇H₃₅ |
| 4-chlorophenyl | 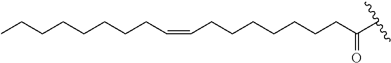 [SMILES: *C(=O)CCCCCCC/C=C\CCCCCCCC] |
| 4-chlorophenyl | 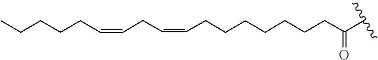 [SMILES: *C(=O)CCCCCCC/C=C\C/C=C\CCCCC] |
| 4-chlorophenyl | 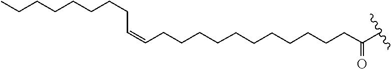 [SMILES: *C(=O)CCCCCCCCCCC/C=C\CCCCCCCC] |
| 4-chlorophenyl | 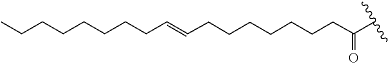 [SMILES: *C(=O)CCCCCCC/C=C/CCCCCCCC] |
| 4-chlorophenyl | 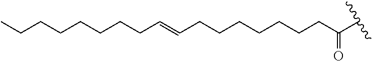 [SMILES: *C(=O)CCCCCCC/C=C/CCCCCCCC] |
| 4-chlorophenyl | tBu |
| 4-chlorophenyl | phenyl |
| 4-chlorophenyl | 4-chlorophenyl |
| 4-chlorophenyl | 3,4-dichlorophenyl |
| 4-chlorophenyl | 4-methylphenyl |
| 4-chlorophenyl | 4-methoxyphenyl |
| 3,4-dichlorophenyl | Me |
| 3,4-dichlorophenyl | —C₇H₁₅ |
| 3,4-dichlorophenyl | —C₉H₁₉ |
| 3,4-dichlorophenyl | —C₁₁H₂₃ |
| 3,4-dichlorophenyl | —C₁₃H₂₇ |
| 3,4-dichlorophenyl | —C₁₅H₃₁ |
| 3,4-dichlorophenyl | —C₁₇H₃₅ |
| 3,4-dichlorophenyl | 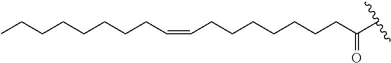 [SMILES: *C(=O)CCCCCCC/C=C\CCCCCCCC] |
| 3,4-dichlorophenyl | 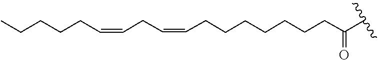 [SMILES: *C(=O)CCCCCCC/C=C\C/C=C\CCCCC] |
| 3,4-dichlorophenyl | 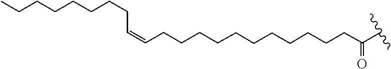 [SMILES: *C(=O)CCCCCCCCCCC/C=C\CCCCCCCC] |
| 3,4-dichlorophenyl | 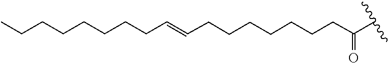 [SMILES: *C(=O)CCCCCCC/C=C/CCCCCCCC] |
| 3,4-dichlorophenyl | 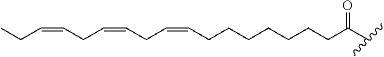 [SMILES: *C(=O)CCCCCCC/C=C\C/C=C\C/C=C\CC] |
| 3,4-dichlorophenyl | tBu |
| 3,4-dichlorophenyl | phenyl |
| 3,4-dichlorophenyl | 4-chlorophenyl |
| 3,4-dichlorophenyl | 3,4-dichlorophenyl |
| 3,4-dichlorophenyl | 4-methylphenyl |
| 3,4-dichlorophenyl | 4-methoxyphenyl |
| 4-methylphenyl | Me |
| 4-methylphenyl | —C₇H₁₅ |
| 4-methylphenyl | —C₉H₁₉ |
| 4-methylphenyl | —C₁₁H₂₃ |
| 4-methylphenyl | —C₁₃H₂₇ |
| 4-methylphenyl | —C₁₅H₃₁ |
| 4-methylphenyl | —C₁₇H₃₅ |
| 4-methylphenyl | 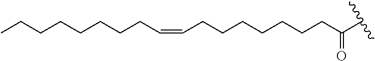 [SMILES: *C(=O)CCCCCCC/C=C\CCCCCCCC] |
| 4-methylphenyl | 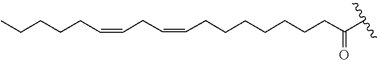 [SMILES: *C(=O)CCCCCCC/C=C\C/C=C\CCCCC] |
| 4-methylphenyl | 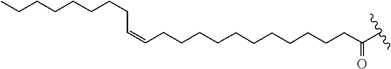 [SMILES: *C(=O)CCCCCCCCCCC/C=C\CCCCCCCC] |
| 4-methylphenyl | 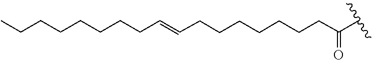 [SMILES: *C(=O)CCCCCCC/C=C/CCCCCCCC] |
| 4-methylphenyl | 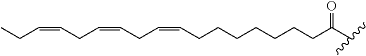 [SMILES: *C(=O)CCCCCCC/C=C\C/C=C\C/C=C\CC] |
| 4-methylphenyl | tBu |
| 4-methylphenyl | phenyl |
| 4-methylphenyl | 4-chlorophenyl |
| 4-methylphenyl | 3,4-dichlorophenyl |
| 4-methylphenyl | 4-methylphenyl |
| 4-methylphenyl | 4-methoxyphenyl |
| 4-methoxyphenyl | Me |
| 4-methoxyphenyl | —C₇H₁₅ |
| 4-methoxyphenyl | —C₉H₁₉ |
| 4-methoxyphenyl | —C₁₁H₂₃ |
| 4-methoxyphenyl | —C₁₃H₂₇ |
| 4-methoxyphenyl | —C₁₅H₃₁ |
| 4-methoxyphenyl | —C₁₇H₃₅ |
| 4-methoxyphenyl | 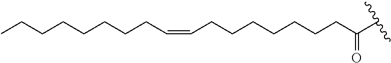 [SMILES: *C(=O)CCCCCCC/C=C\CCCCCCCC] |
| 4-methoxyphenyl | 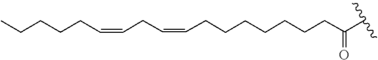 [SMILES: *C(=O)CCCCCCC/C=C\C/C=C\CCCCC] |
| 4-methoxyphenyl | 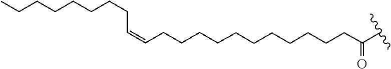 [SMILES: *C(=O)CCCCCCCCCCC/C=C\CCCCCCCC] |
| 4-methoxyphenyl | 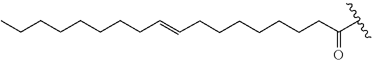 [SMILES: *C(=O)CCCCCCC/C=C/CCCCCCCC] |
| 4-methoxyphenyl | 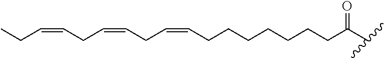 [SMILES: *C(=O)CCCCCCC/C=C\C/C=C\C/C=C\CC] |
| 4-methoxyphenyl | tBu |
| 4-methoxyphenyl | phenyl |
| 4-methoxyphenyl | 4-chlorophenyl |
| 4-methoxyphenyl | 3,4-dichlorophenyl |
| 4-methoxyphenyl | 4-methylphenyl |
| 4-methoxyphenyl | 4-methoxyphenyl |
| 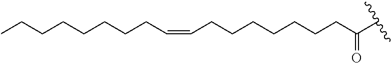 [SMILES: *C(=O)CCCCCCC/C=C\CCCCCCCC] | Me |
| 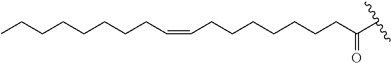 [SMILES: *C(=O)CCCCCCC/C=C\CCCCCCCC] | —C₇H₁₅ |
| 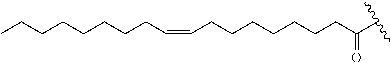 [SMILES: *C(=O)CCCCCCC/C=C\CCCCCCCC] | —C₉H₁₉ |
| 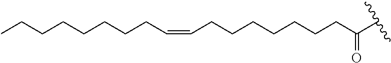 [SMILES: *C(=O)CCCCCCC/C=C\CCCCCCCC] | —C₁₁H₂₃ |
| 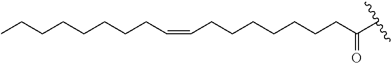 [SMILES: *C(=O)CCCCCCC/C=C\CCCCCCCC] | —C₁₃H₂₇ |
| 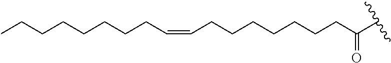 [SMILES: *C(=O)CCCCCCC/C=C\CCCCCCCC] | —C₁₅H₃₁ |
| 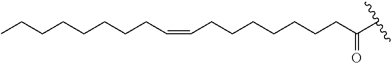 [SMILES: *C(=O)CCCCCCC/C=C\CCCCCCCC] | —C₁₇H₃₅ |
| 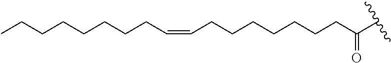 [SMILES: *C(=O)CCCCCCC/C=C\CCCCCCCC] | 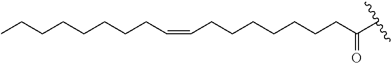 [SMILES: *C(=O)CCCCCCC/C=C\CCCCCCCC] |
| 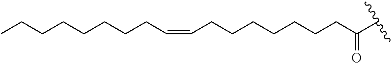 [SMILES: *C(=O)CCCCCCC/C=C\CCCCCCCC] | 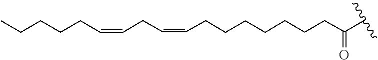 [SMILES: *C(=O)CCCCCCC/C=C\C/C=C\CCCCC] |
| 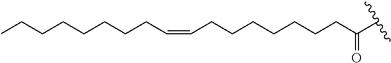 [SMILES: *C(=O)CCCCCCC/C=C\CCCCCCCC] | 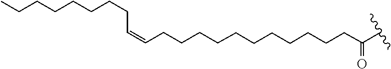 [SMILES: *C(=O)CCCCCCCCCCC/C=C\CCCCCCCC] |
| 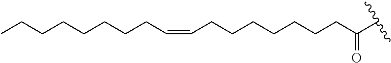 [SMILES: *C(=O)CCCCCCC/C=C\CCCCCCCC] | 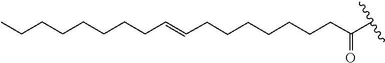 [SMILES: *C(=O)CCCCCCC/C=C/CCCCCCCC] |
| 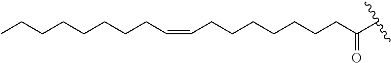 [SMILES: *C(=O)CCCCCCC/C=C\CCCCCCCC] | 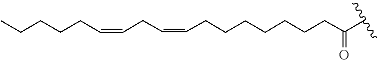 [SMILES: *C(=O)CCCCCCC/C=C\C/C=C\CCCCC] |
| 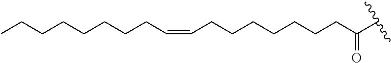 [SMILES: *C(=O)CCCCCCC/C=C\CCCCCCCC] | tBu |
| 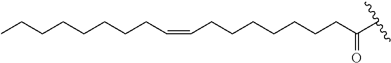 [SMILES: *C(=O)CCCCCCC/C=C\CCCCCCCC] | phenyl |
| 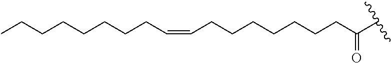 [SMILES: *C(=O)CCCCCCC/C=C\CCCCCCCC] | 4-chlorophenyl |
| 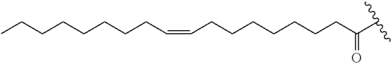 [SMILES: *C(=O)CCCCCCC/C=C\CCCCCCCC] | 3,4-dichlorophenyl |
| 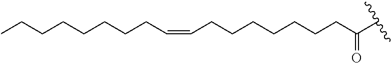 [SMILES: *C(=O)CCCCCCC/C=C\CCCCCCCC] | 4-methylphenyl |
| 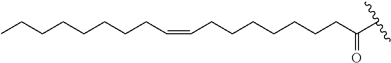 [SMILES: *C(=O)CCCCCCC/C=C\CCCCCCCC] | 4-methoxyphenyl |
| 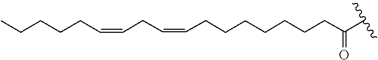 [SMILES: *C(=O)CCCCCCC/C=C\C/C=C\CCCCC] | Me |
| 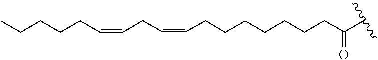 [SMILES: *C(=O)CCCCCCC/C=C\C/C=C\CCCCC] | —C₇H₁₅ |
| 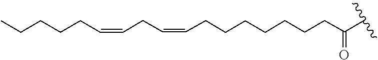 [SMILES: *C(=O)CCCCCCC/C=C\C/C=C\CCCCC] | —C₉H₁₉ |
| 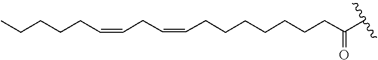 [SMILES: *C(=O)CCCCCCC/C=C\C/C=C\CCCCC] | —C₁₁H₂₃ |
| 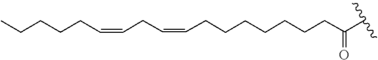 [SMILES: *C(=O)CCCCCCC/C=C\C/C=C\CCCCC] | —C₁₃H₂₇ |
| 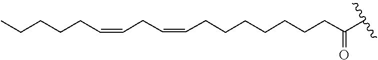 [SMILES: *C(=O)CCCCCCC/C=C\C/C=C\CCCCC] | —C₁₅H₃₁ |
| 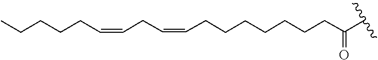 [SMILES: *C(=O)CCCCCCC/C=C\C/C=C\CCCCC] | —C₁₇H₃₅ |
| 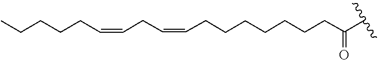 [SMILES: *C(=O)CCCCCCC/C=C\C/C=C\CCCCC] | 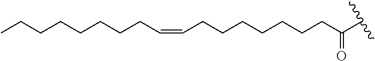 [SMILES: *C(=O)CCCCCCC/C=C\CCCCCCCC] |
| 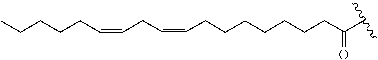 [SMILES: *C(=O)CCCCCCC/C=C\C/C=C\CCCCC] | 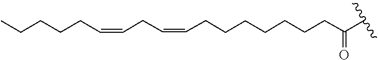 [SMILES: *C(=O)CCCCCCC/C=C\C/C=C\CCCCC] |
| 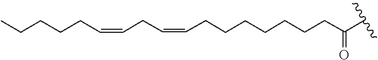 [SMILES: *C(=O)CCCCCCC/C=C\C/C=C\CCCCC] | 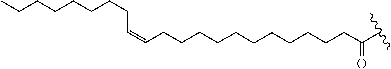 [SMILES: *C(=O)CCCCCCCCCCC/C=C\CCCCCCCC] |
| 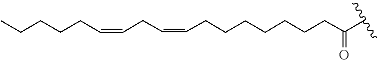 [SMILES: *C(=O)CCCCCCC/C=C\C/C=C\CCCCC] | 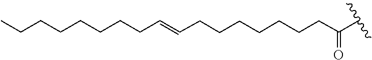 [SMILES: *C(=O)CCCCCCC/C=C/CCCCCCCC] |
| 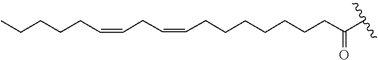 [SMILES: *C(=O)CCCCCCC/C=C\C/C=C\CCCCC] | 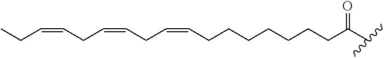 [SMILES: *C(=O)CCCCCCC/C=C\C/C=C\C/C=C\CC] |
| 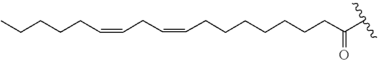 [SMILES: *C(=O)CCCCCCC/C=C\C/C=C\CCCCC] | tBu |
| 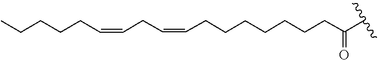 [SMILES: *C(=O)CCCCCCC/C=C\C/C=C\CCCCC] | phenyl |
| 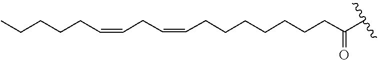 [SMILES: *C(=O)CCCCCCC/C=C\C/C=C\CCCCC] | 4-chlorophenyl |
| 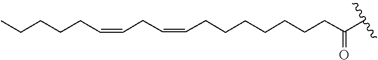 [SMILES: *C(=O)CCCCCCC/C=C\C/C=C\CCCCC] | 3,4-dichlorophenyl |
| 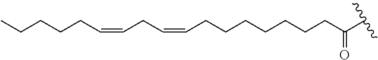 [SMILES: *C(=O)CCCCCCC/C=C\C/C=C\CCCCC] | 4-methylphenyl |
| 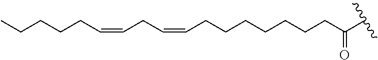 [SMILES: *C(=O)CCCCCCC/C=C\C/C=C\CCCCC] | 4-methoxyphenyl |
| 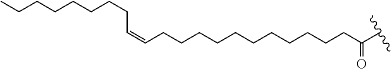 [SMILES: *C(=O)CCCCCCCCCCC/C=C\CCCCCCCC] | Me |
| 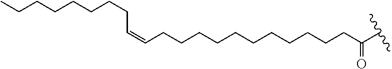 [SMILES: *C(=O)CCCCCCCCCCC/C=C\CCCCCCCC] | —C₇H₁₅ |
| 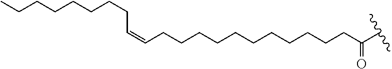 [SMILES: *C(=O)CCCCCCCCCCC/C=C\CCCCCCCC] | —C₉H₁₉ |
| 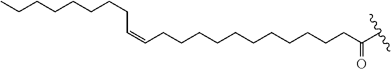 [SMILES: *C(=O)CCCCCCCCCCC/C=C\CCCCCCCC] | —C₁₁H₂₃ |
| 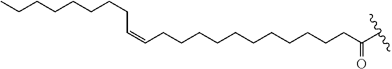 [SMILES: *C(=O)CCCCCCCCCCC/C=C\CCCCCCCC] | —C₁₃H₂₇ |
| 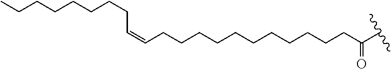 [SMILES: *C(=O)CCCCCCCCCCC/C=C\CCCCCCCC] | —C₁₅H₃₁ |
| 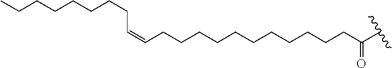 [SMILES: *C(=O)CCCCCCCCCCC/C=C\CCCCCCCC] | —C₁₇H₃₅ |
| 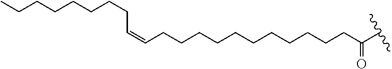 [SMILES: *C(=O)CCCCCCCCCCC/C=C\CCCCCCCC] | 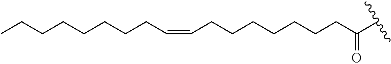 |
| 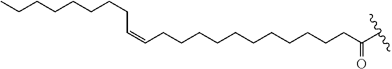 [SMILES: *C(=O)CCCCCCCCCCC/C=C\CCCCCCCC] | 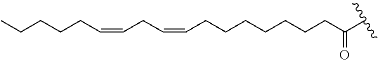 [SMILES: *C(=O)CCCCCCC/C=C\C/C=C\CCCCC] |
| 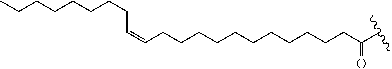 [SMILES: *C(=O)CCCCCCCCCCC/C=C\CCCCCCCC] | 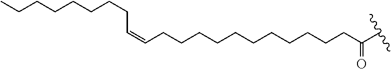 [SMILES: *C(=O)CCCCCCCCCCC/C=C\CCCCCCCC] |
| 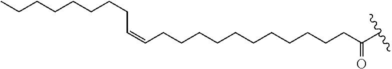 [SMILES: *C(=O)CCCCCCCCCCC/C=C\CCCCCCCC] | 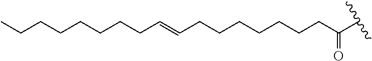 [SMILES: *C(=O)CCCCCCC/C=C/CCCCCCCC] |
| 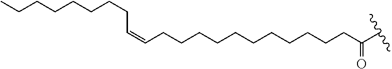 [SMILES: *C(=O)CCCCCCCCCCC/C=C\CCCCCCCC] | 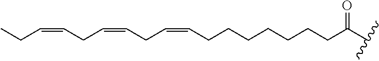 [SMILES: *C(=O)CCCCCCC/C=C\C/C=C\C/C=C\CC] |
| 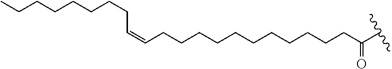 [SMILES: *C(=O)CCCCCCCCCCC/C=C\CCCCCCCC] | tBu |
| 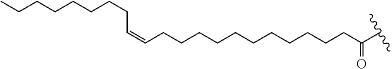 [SMILES: *C(=O)CCCCCCCCCCC/C=C\CCCCCCCC] | phenyl |
| 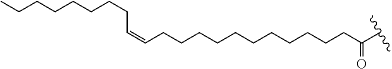 [SMILES: *C(=O)CCCCCCCCCCC/C=C\CCCCCCCC] | 4-chlorophenyl |
| 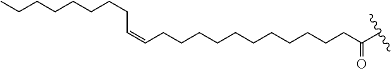 [SMILES: *C(=O)CCCCCCCCCCC/C=C\CCCCCCCC] | 3,4-dichlorophenyl |
| 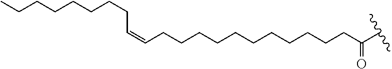 [SMILES: *C(=O)CCCCCCCCCCC/C=C\CCCCCCCC] | 4-methylphenyl |
| 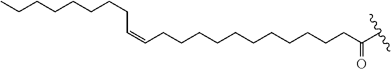 [SMILES: *C(=O)CCCCCCCCCCC/C=C\CCCCCCCC] | 4-methoxyphenyl |
| 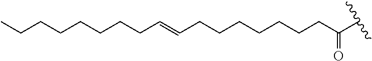 [SMILES: *C(=O)CCCCCCC/C=C/CCCCCCCC] | Me |
| 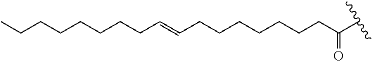 [SMILES: *C(=O)CCCCCCC/C=C/CCCCCCCC] | —C₇H₁₅ |
| 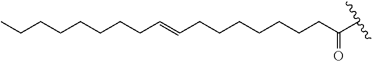 [SMILES: *C(=O)CCCCCCC/C=C/CCCCCCCC] | —C₉H₁₉ |
| 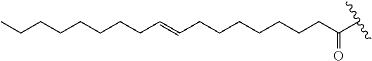 [SMILES: *C(=O)CCCCCCC/C=C/CCCCCCCC] | —C₁₁H₂₃ |
| 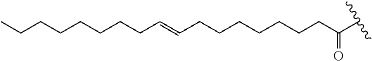 [SMILES: *C(=O)CCCCCCC/C=C/CCCCCCCC] | —C₁₃H₂₇ |
| 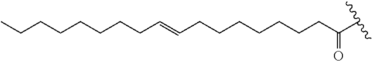 [SMILES: *C(=O)CCCCCCC/C=C/CCCCCCCC] | —C₁₅H₃₁ |
| 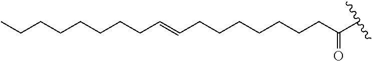 [SMILES: *C(=O)CCCCCCC/C=C/CCCCCCCC] | —C₁₇H₃₅ |
| 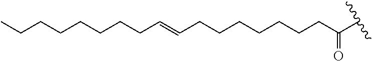 [SMILES: *C(=O)CCCCCCC/C=C/CCCCCCCC] | 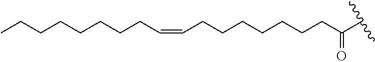 |
| 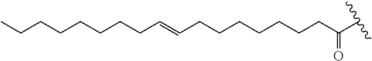 [SMILES: *C(=O)CCCCCCC/C=C/CCCCCCCC] | 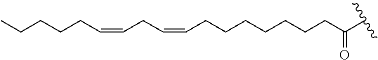 [SMILES: *C(=O)CCCCCCC/C=C\C/C=C\CCCCC] |
| 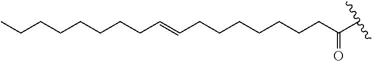 [SMILES: *C(=O)CCCCCCC/C=C/CCCCCCCC] | 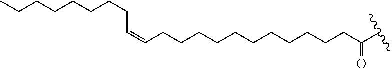 [SMILES: *C(=O)CCCCCCCCCCC/C=C\CCCCCCCC] |
| 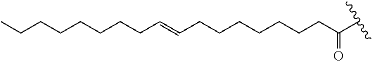 [SMILES: *C(=O)CCCCCCC/C=C/CCCCCCCC] | 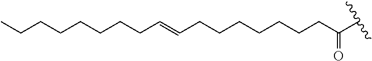 [SMILES: *C(=O)CCCCCCC/C=C/CCCCCCCC] |
| 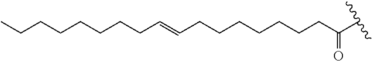 [SMILES: *C(=O)CCCCCCC/C=C/CCCCCCCC] | 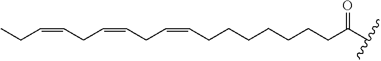 [SMILES: *C(=O)CCCCCCC/C=C\C/C=C\C/C=C\CC] |
| 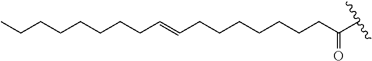 [SMILES: *C(=O)CCCCCCC/C=C/CCCCCCCC] | tBu |
| 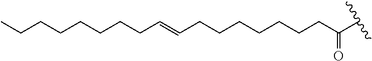 [SMILES: *C(=O)CCCCCCC/C=C/CCCCCCCC] | phenyl |
| 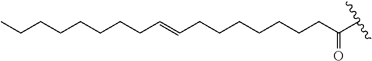 [SMILES: *C(=O)CCCCCCC/C=C/CCCCCCCC] | 4-chlorophenyl |
| 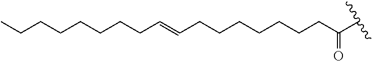 [SMILES: *C(=O)CCCCCCC/C=C/CCCCCCCC] | 3,4-dichlorophenyl |
| 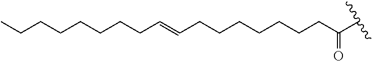 [SMILES: *C(=O)CCCCCCC/C=C/CCCCCCCC] | 4-methylphenyl |
| 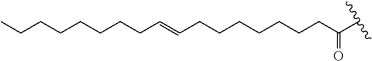 [SMILES: *C(=O)CCCCCCC/C=C/CCCCCCCC] | 4-methoxyphenyl |
| 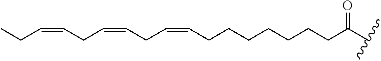 [SMILES: *C(=O)CCCCCCC/C=C\C/C=C\C/C=C\CC] | Me |
| 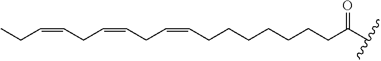 [SMILES: *C(=O)CCCCCCC/C=C\C/C=C\C/C=C\CC] | —C₇H₁₅ |
| 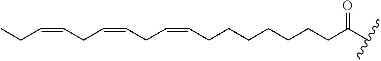 [SMILES: *C(=O)CCCCCCC/C=C\C/C=C\C/C=C\CC] | —C₉H₁₉ |
| 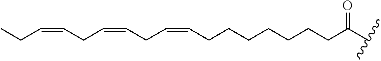 [SMILES: *C(=O)CCCCCCC/C=C\C/C=C\C/C=C\CC] | —C₁₁H₂₃ |
| 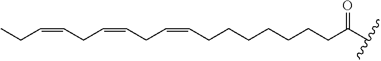 [SMILES: *C(=O)CCCCCCC/C=C\C/C=C\C/C=C\CC] | —C₁₃H₂₇ |
| 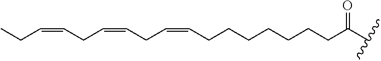 [SMILES: *C(=O)CCCCCCC/C=C\C/C=C\C/C=C\CC] | —C₁₅H₃₁ |
| 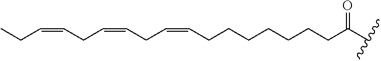 [SMILES: *C(=O)CCCCCCC/C=C\C/C=C\C/C=C\CC] | —C₁₇H₃₅ |
| 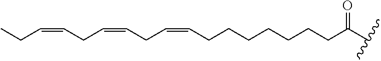 [SMILES: *C(=O)CCCCCCC/C=C\C/C=C\C/C=C\CC] | 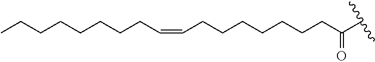 |
| 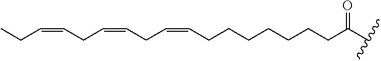 [SMILES: *C(=O)CCCCCCC/C=C\C/C=C\C/C=C\CC] | 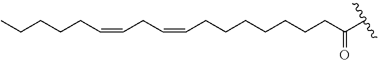 [SMILES: *C(=O)CCCCCCC/C=C\C/C=C\CCCCC] |
| 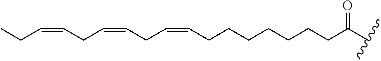 [SMILES: *C(=O)CCCCCCC/C=C\C/C=C\C/C=C\CC] | 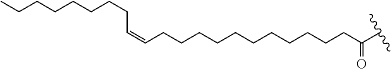 [SMILES: *C(=O)CCCCCCCCCCC/C=C\CCCCCCCC] |
| 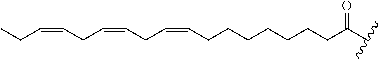 [SMILES: *C(=O)CCCCCCC/C=C\C/C=C\C/C=C\CC] | 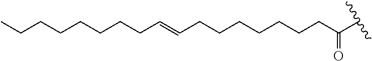 [SMILES: *C(=O)CCCCCCC/C=C/CCCCCCCC] |
| 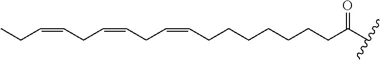 [SMILES: *C(=O)CCCCCCC/C=C\C/C=C\C/C=C\CC] | 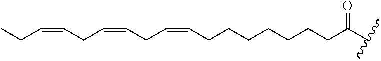 [SMILES: *C(=O)CCCCCCC/C=C\C/C=C\C/C=C\CC] |
| 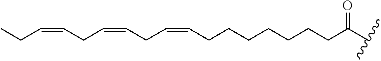 [SMILES: *C(=O)CCCCCCC/C=C\C/C=C\C/C=C\CC] | tBu |
| 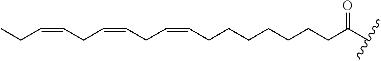 [SMILES: *C(=O)CCCCCCC/C=C\C/C=C\C/C=C\CC] | phenyl |
| 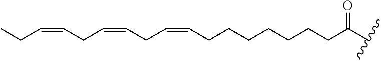 [SMILES: *C(=O)CCCCCCC/C=C\C/C=C\C/C=C\CC] | 4-chlorophenyl |
| 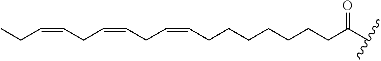 [SMILES: *C(=O)CCCCCCC/C=C\C/C=C\C/C=C\CC] | 3,4-dichlorophenyl |
| 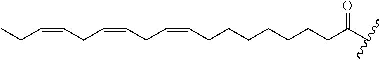 [SMILES: *C(=O)CCCCCCC/C=C\C/C=C\C/C=C\CC] | 4-methylphenyl |
| 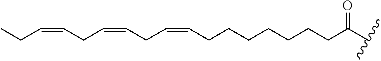 [SMILES: *C(=O)CCCCCCC/C=C\C/C=C\C/C=C\CC] | 4-methoxyphenyl |
| H | 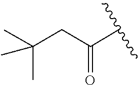 [SMILES: *C(=O)CC(C)(C)C] |
| H | 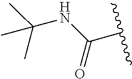 [SMILES: *C(=O)NC(C)(C)C] |
| H | 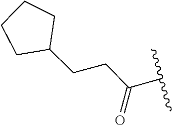 [SMILES: *C(=O)CCC1CCCC1] |
| H | 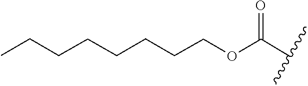 [SMILES: *C(=O)OCCCCCCCC] |
| H | 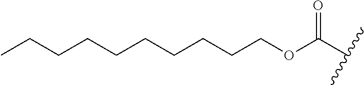 [SMILES: *C(=O)OCCCCCCCCCC] |
| H | 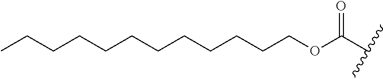 [SMILES: *C(=O)OCCCCCCCCCCCC] |
| H | 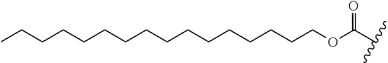 [SMILES: *C(=O)OCCCCCCCCCCCCCCCC] |
| H | 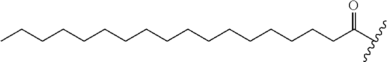 [SMILES: *C(=O)CCCCCCCCCCCCCCCCC] |
| H | 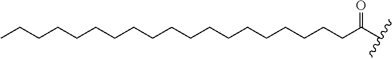 [SMILES: *C(=O)CCCCCCCCCCCCCCCCCCC] |
| H | 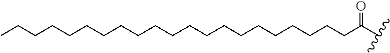 [SMILES: *C(=O)CCCCCCCCCCCCCCCCCCCCC] |
| H | 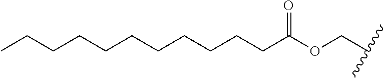 [SMILES: *COC(=O)CCCCCCCCCCC] |
| H | 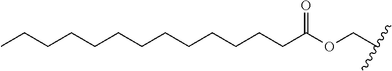 [SMILES: *COC(=O)CCCCCCCCCCCCC] |
| H | 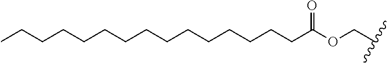 [SMILES: *COC(=O)CCCCCCCCCCCCCCC] |
| H | 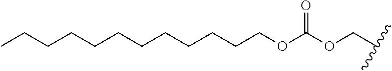 [SMILES: *COC(=O)OCCCCCCCCCCCC] |
| H | 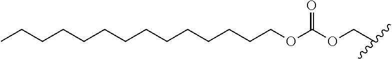 [SMILES: *COC(=O)OCCCCCCCCCCCCCC] |
| H | 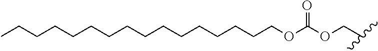 [SMILES: *COC(=O)OCCCCCCCCCCCCCCCC] |
| Me | 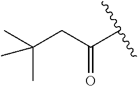 [SMILES: *C(=O)CC(C)(C)C] |
| Me | 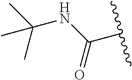 [SMILES: *C(=O)NC(C)(C)C] |
| Me | 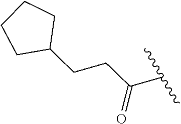 [SMILES: *C(=O)CCC1CCCC1] |
| Me | 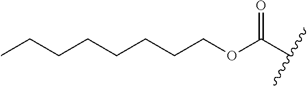 [SMILES: *C(=O)OCCCCCCCC] |
| Me | 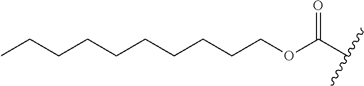 [SMILES: *C(=O)OCCCCCCCCCC] |
| Me | 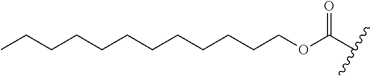 [SMILES: *C(=O)OCCCCCCCCCCCC] |
| Me | 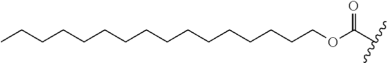 [SMILES: *C(=O)OCCCCCCCCCCCCCCCC] |
| Me | 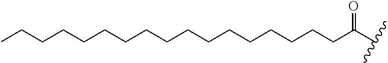 [SMILES: *C(=O)CCCCCCCCCCCCCCCCC] |
| Me | 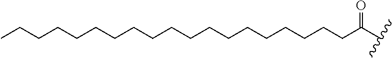 [SMILES: *C(=O)CCCCCCCCCCCCCCCCCCC] |
| Me | 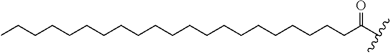 [SMILES: *C(=O)CCCCCCCCCCCCCCCCCCCCC] |
| Me | 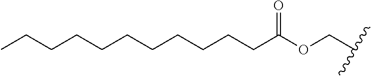 [SMILES: *COC(=O)CCCCCCCCCCC] |
| Me | 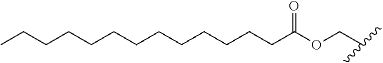 [SMILES: *COC(=O)CCCCCCCCCCCCC] |
| Me | 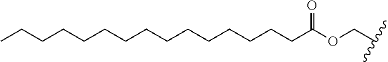 [SMILES: *COC(=O)CCCCCCCCCCCCCCC] |
| Me | 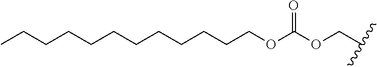 [SMILES: *COC(=O)OCCCCCCCCCCCC] |
| Me | 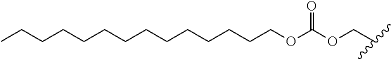 [SMILES: *COC(=O)OCCCCCCCCCCCCCC] |
| Me | 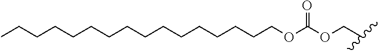 [SMILES: *COC(=O)OCCCCCCCCCCCCCCCC] |
| —C₇H₁₅ | 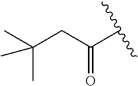 [SMILES: *C(=O)CC(C)(C)C] |
| —C₇H₁₅ | 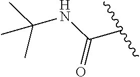 [SMILES: *C(=O)NC(C)(C)C] |
| —C₇H₁₅ | 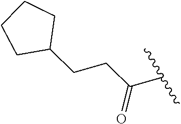 [SMILES: *C(=O)CCC1CCCC1] |
| —C₇H₁₅ | 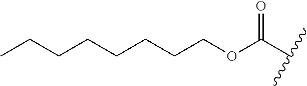 [SMILES: *C(=O)OCCCCCCCC] |
| —C₇H₁₅ | 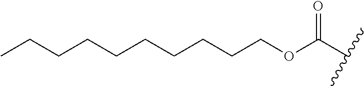 [SMILES: *C(=O)OCCCCCCCCCC] |
| —C₇H₁₅ | 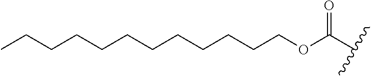 [SMILES: *C(=O)OCCCCCCCCCCCC] |
| —C₇H₁₅ | 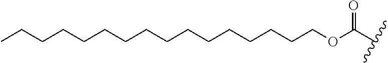 [SMILES: *C(=O)OCCCCCCCCCCCCCCCC] |
| —C₇H₁₅ | 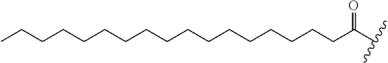 [SMILES: *C(=O)CCCCCCCCCCCCCCCCC] |
| —C₇H₁₅ | 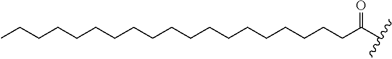 [SMILES: *C(=O)CCCCCCCCCCCCCCCCCCC] |
| —C₇H₁₅ | 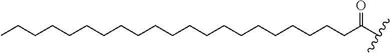 [SMILES: *C(=O)CCCCCCCCCCCCCCCCCCCCC] |
| —C₇H₁₅ | 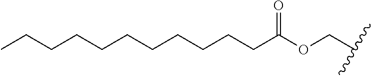 [SMILES: *COC(=O)CCCCCCCCCCC] |
| —C₇H₁₅ | 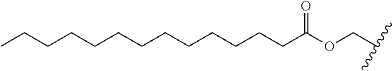 [SMILES: *COC(=O)CCCCCCCCCCCCC] |
| —C₇H₁₅ | 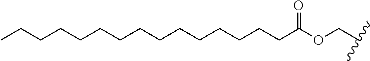 [SMILES: *COC(=O)CCCCCCCCCCCCCCC] |
| —C₇H₁₅ | 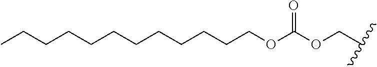 [SMILES: *COC(=O)OCCCCCCCCCCCC] |
| —C₇H₁₅ | 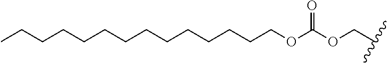 [SMILES: *COC(=O)OCCCCCCCCCCCCCC] |
| —C₇H₁₅ | 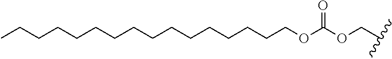 [SMILES: *COC(=O)OCCCCCCCCCCCCCCCC] |
| —C₉H₁₉ | 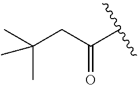 [SMILES: *C(=O)CC(C)(C)C] |
| —C₉H₁₉ | 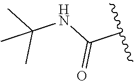 [SMILES: *C(=O)NC(C)(C)C] |
| —C₉H₁₉ | 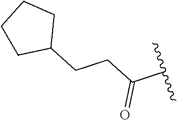 [SMILES: *C(=O)CCC1CCCC1] |
| —C₉H₁₉ | 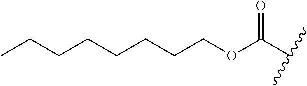 [SMILES: *C(=O)OCCCCCCCC] |
| —C₉H₁₉ | 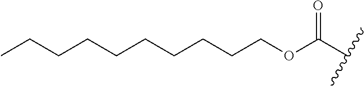 [SMILES: *C(=O)OCCCCCCCCCC] |
| —C₉H₁₉ | 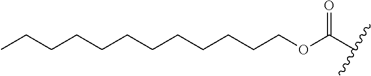 [SMILES: *C(=O)OCCCCCCCCCCCC] |
| —C₉H₁₉ | 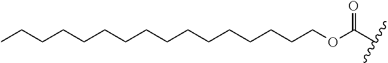 [SMILES: *C(=O)OCCCCCCCCCCCCCCCC] |
| —C₉H₁₉ | 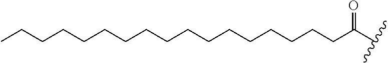 [SMILES: *C(=O)CCCCCCCCCCCCCCCCC] |
| —C₉H₁₉ | 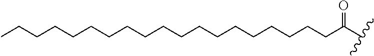 [SMILES: *C(=O)CCCCCCCCCCCCCCCCCCC] |
| —C₉H₁₉ | 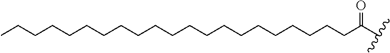 [SMILES: *C(=O)CCCCCCCCCCCCCCCCCCCCC] |
| —C₉H₁₉ | 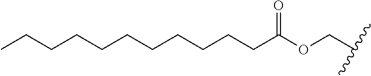 [SMILES: *COC(=O)CCCCCCCCCCC] |
| —C₉H₁₉ | 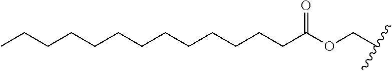 [SMILES: *COC(=O)CCCCCCCCCCCCC] |
| —C₉H₁₉ | 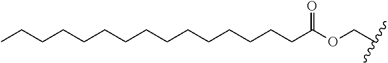 [SMILES: *COC(=O)CCCCCCCCCCCCCCC] |
| —C₉H₁₉ | 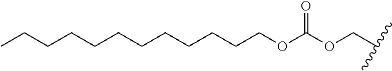 [SMILES: *COC(=O)OCCCCCCCCCCCC] |
| —C₉H₁₉ | 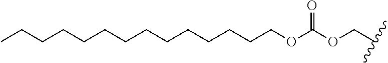 [SMILES: *COC(=O)OCCCCCCCCCCCCCC] |
| —C₉H₁₉ | 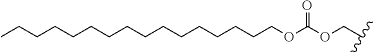 [SMILES: *COC(=O)OCCCCCCCCCCCCCCCC] |
| —C₁₁H₂₃ | 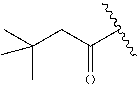 [SMILES: *C(=O)CC(C)(C)C] |
| —C₁₁H₂₃ | 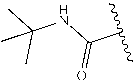 [SMILES: *C(=O)NC(C)(C)C] |
| —C₁₁H₂₃ | 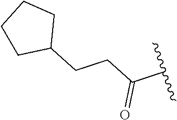 [SMILES: *C(=O)CCC1CCCC1] |
| —C₁₁H₂₃ | 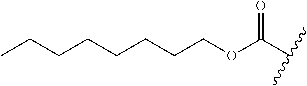 [SMILES: *C(=O)OCCCCCCCC] |
| —C₁₁H₂₃ | 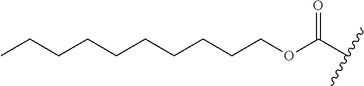 [SMILES: *C(=O)OCCCCCCCCCC] |
| —C₁₁H₂₃ | 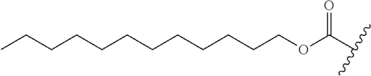 [SMILES: *C(=O)OCCCCCCCCCCCC] |
| —C₁₁H₂₃ | 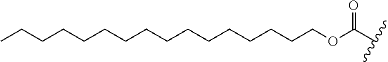 [SMILES: *C(=O)OCCCCCCCCCCCCCCCC] |
| —C₁₁H₂₃ | 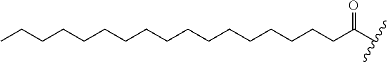 [SMILES: *C(=O)CCCCCCCCCCCCCCCCC] |
| —C₁₁H₂₃ | 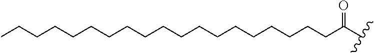 [SMILES: *C(=O)CCCCCCCCCCCCCCCCCCC] |
| —C₁₁H₂₃ | 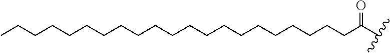 [SMILES: *C(=O)CCCCCCCCCCCCCCCCCCCCC] |
| —C₁₁H₂₃ | 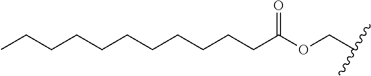 [SMILES: *COC(=O)CCCCCCCCCCC] |
| —C₁₁H₂₃ | 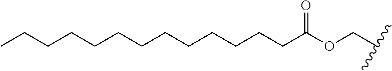 [SMILES: *COC(=O)CCCCCCCCCCCCC] |
| —C₁₁H₂₃ | 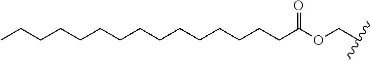 [SMILES: *COC(=O)CCCCCCCCCCCCCCC] |
| —C₁₁H₂₃ | 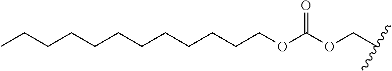 [SMILES: *COC(=O)OCCCCCCCCCCCC] |
| —C₁₁H₂₃ | 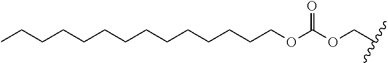 [SMILES: *COC(=O)OCCCCCCCCCCCCCC] |
| —C₁₁H₂₃ | 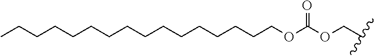 [SMILES: *COC(=O)OCCCCCCCCCCCCCCCC] |
| —C₁₃H₂₇ | 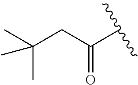 [SMILES: *C(=O)CC(C)(C)C] |
| —C₁₃H₂₇ | 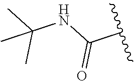 [SMILES: *C(=O)NC(C)(C)C] |
| —C₁₃H₂₇ | 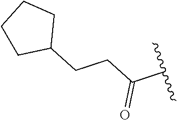 [SMILES: *C(=O)CCC1CCCC1] |
| —C₁₃H₂₇ | 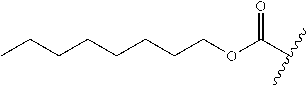 [SMILES: *C(=O)OCCCCCCCC] |
| —C₁₃H₂₇ | 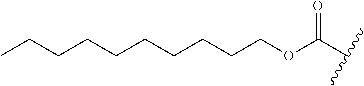 [SMILES: *C(=O)OCCCCCCCCCC] |
| —C₁₃H₂₇ | 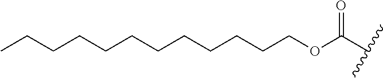 [SMILES: *C(=O)OCCCCCCCCCCCC] |
| —C₁₃H₂₇ | 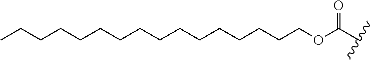 [SMILES: *C(=O)OCCCCCCCCCCCCCCCC] |
| —C₁₃H₂₇ | 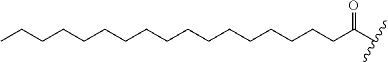 [SMILES: *C(=O)CCCCCCCCCCCCCCCCC] |
| —C₁₃H₂₇ | 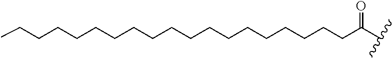 [SMILES: *C(=O)CCCCCCCCCCCCCCCCCCC] |
| —C₁₃H₂₇ | 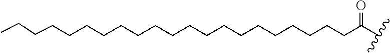 [SMILES: *C(=O)CCCCCCCCCCCCCCCCCCCCC] |
| —C₁₃H₂₇ | 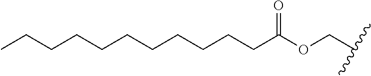 [SMILES: *COC(=O)CCCCCCCCCCC] |
| —C₁₃H₂₇ | 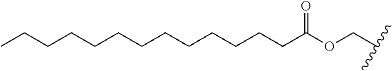 [SMILES: *COC(=O)CCCCCCCCCCCCC] |
| —C₁₃H₂₇ | 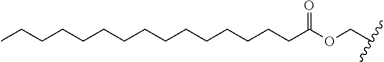 [SMILES: *COC(=O)CCCCCCCCCCCCCCC] |
| —C₁₃H₂₇ | 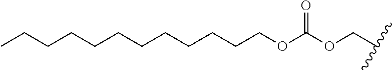 [SMILES: *COC(=O)OCCCCCCCCCCCC] |
| —C₁₃H₂₇ | 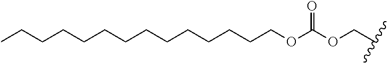 [SMILES: *COC(=O)OCCCCCCCCCCCCCC] |
| —C₁₃H₂₇ | 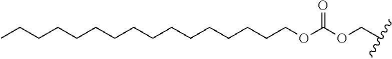 [SMILES: *COC(=O)OCCCCCCCCCCCCCCCC] |
| —C₁₅H₃₁ | 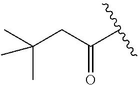 [SMILES: *C(=O)CC(C)(C)C] |
| —C₁₅H₃₁ | 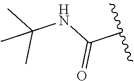 [SMILES: *C(=O)NC(C)(C)C] |
| —C₁₅H₃₁ | 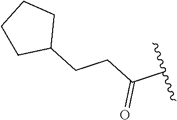 [SMILES: *C(=O)CCC1CCCC1] |
| —C₁₅H₃₁ | 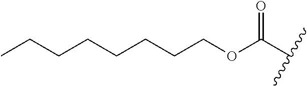 [SMILES: *C(=O)OCCCCCCCC] |
| —C₁₅H₃₁ | 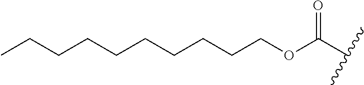 [SMILES: *C(=O)OCCCCCCCCCC] |
| —C₁₅H₃₁ | 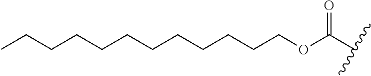 [SMILES: *C(=O)OCCCCCCCCCCCC] |
| —C₁₅H₃₁ | 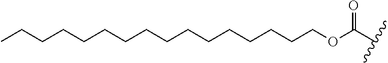 [SMILES: *C(=O)OCCCCCCCCCCCCCCCC] |
| —C₁₅H₃₁ | 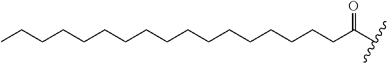 [SMILES: *C(=O)CCCCCCCCCCCCCCCCC] |
| —C₁₅H₃₁ | 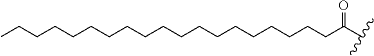 [SMILES: *C(=O)CCCCCCCCCCCCCCCCCCC] |
| —C₁₅H₃₁ | 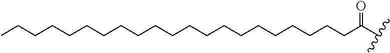 [SMILES: *C(=O)CCCCCCCCCCCCCCCCCCCCC] |
| —C₁₅H₃₁ | 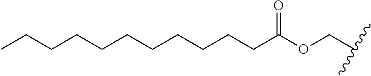 [SMILES: *COC(=O)CCCCCCCCCCC] |
| —C₁₅H₃₁ | 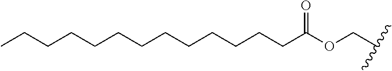 [SMILES: *COC(=O)CCCCCCCCCCCCC] |
| —C₁₅H₃₁ | 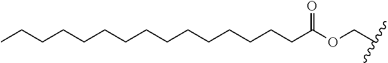 [SMILES: *COC(=O)CCCCCCCCCCCCCCC] |
| —C₁₅H₃₁ | 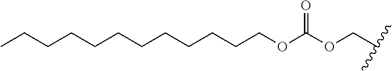 [SMILES: *COC(=O)OCCCCCCCCCCCC] |
| —C₁₅H₃₁ | 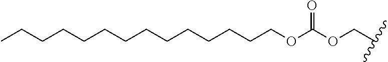 [SMILES: *COC(=O)OCCCCCCCCCCCCCC] |
| —C₁₅H₃₁ | 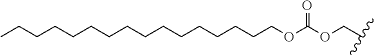 [SMILES: *COC(=O)OCCCCCCCCCCCCCCCC] |
| —C₁₇H₃₅ | 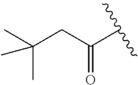 [SMILES: *C(=O)CC(C)(C)C] |
| —C₁₇H₃₅ | 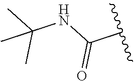 [SMILES: *C(=O)NC(C)(C)C] |
| —C₁₇H₃₅ | 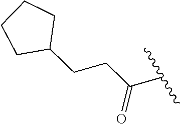 [SMILES: *C(=O)CCC1CCCC1] |
| —C₁₇H₃₅ | 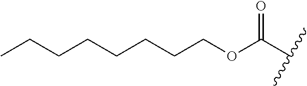 [SMILES: *C(=O)OCCCCCCCC] |
| —C₁₇H₃₅ | 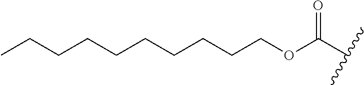 [SMILES: *C(=O)OCCCCCCCCCC] |
| —C₁₇H₃₅ | 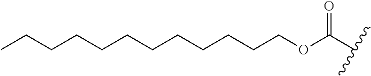 [SMILES: *C(=O)OCCCCCCCCCCCC] |
| —C₁₇H₃₅ | 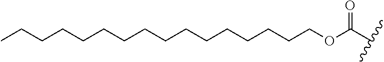 [SMILES: *C(=O)OCCCCCCCCCCCCCCCC] |
| —C₁₇H₃₅ | 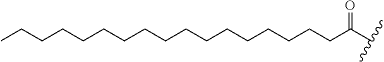 [SMILES: *C(=O)CCCCCCCCCCCCCCCCC] |
| —C₁₇H₃₅ | 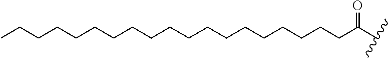 [SMILES: *C(=O)CCCCCCCCCCCCCCCCCCC] |
| —C₁₇H₃₅ | 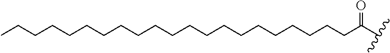 [SMILES: *C(=O)CCCCCCCCCCCCCCCCCCCCC] |
| —C₁₇H₃₅ | 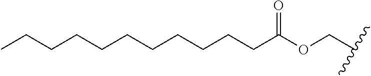 [SMILES: *COC(=O)CCCCCCCCCCC] |
| —C₁₇H₃₅ | 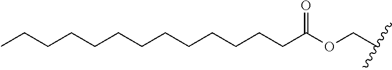 [SMILES: *COC(=O)CCCCCCCCCCCCC] |
| —C₁₇H₃₅ | 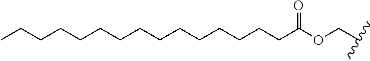 [SMILES: *COC(=O)CCCCCCCCCCCCCCC] |
| —C₁₇H₃₅ | 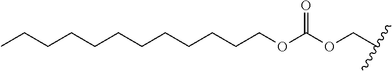 [SMILES: *COC(=O)OCCCCCCCCCCCC] |
| —C₁₇H₃₅ | 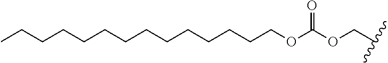 [SMILES: *COC(=O)OCCCCCCCCCCCCCC] |
| —C₁₇H₃₅ | 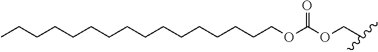 [SMILES: *COC(=O)OCCCCCCCCCCCCCCCC] |
| 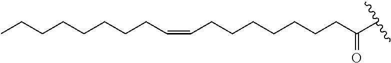 [SMILES: *C(=O)CCCCCCC/C=C\CCCCCCCC] | 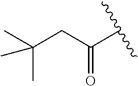 [SMILES: *C(=O)CC(C)(C)C] |
| 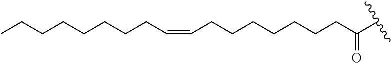 [SMILES: *C(=O)CCCCCCC/C=C\CCCCCCCC] | 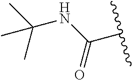 [SMILES: *C(=O)NC(C)(C)C] |
| 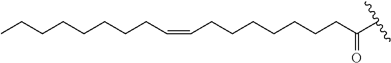 [SMILES: *C(=O)CCCCCCC/C=C\CCCCCCCC] | 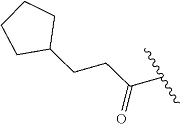 [SMILES: *C(=O)CCC1CCCC1] |
| 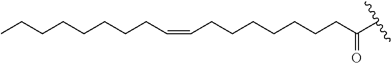 [SMILES: *C(=O)CCCCCCC/C=C\CCCCCCCC] | 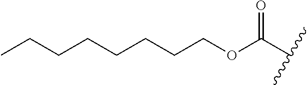 [SMILES: *C(=O)OCCCCCCCC] |
| 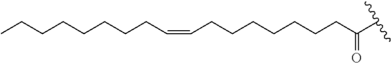 [SMILES: *C(=O)CCCCCCC/C=C\CCCCCCCC] | 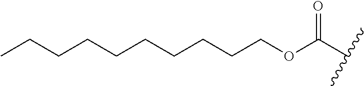 [SMILES: *C(=O)OCCCCCCCCCC] |
| 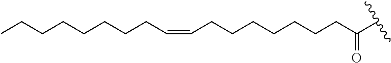 [SMILES: *C(=O)CCCCCCC/C=C\CCCCCCCC] | 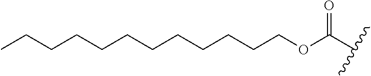 [SMILES: *C(=O)OCCCCCCCCCCCC] |
| 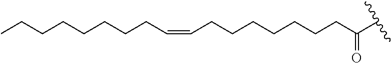 [SMILES: *C(=O)CCCCCCC/C=C\CCCCCCCC] | 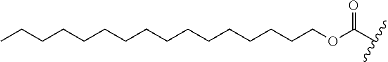 [SMILES: *C(=O)OCCCCCCCCCCCCCCCC] |
| 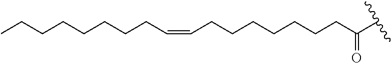 [SMILES: *C(=O)CCCCCCC/C=C\CCCCCCCC] | 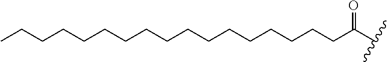 [SMILES: *C(=O)CCCCCCCCCCCCCCCCC] |
| 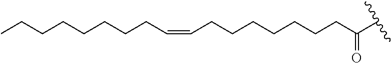 [SMILES: *C(=O)CCCCCCC/C=C\CCCCCCCC] | 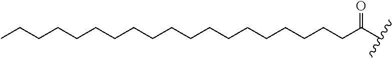 [SMILES: *C(=O)CCCCCCCCCCCCCCCCCCC] |
| 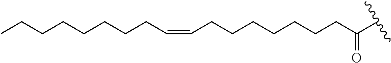 [SMILES: *C(=O)CCCCCCC/C=C\CCCCCCCC] | 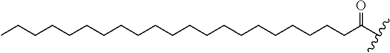 [SMILES: *C(=O)CCCCCCCCCCCCCCCCCCCCC] |
| 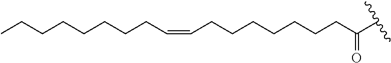 [SMILES: *C(=O)CCCCCCC/C=C\CCCCCCCC] | 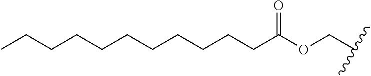 [SMILES: *COC(=O)CCCCCCCCCCC] |
| 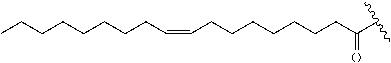 [SMILES: *C(=O)CCCCCCC/C=C\CCCCCCCC] | 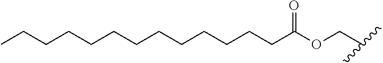 [SMILES: *COC(=O)CCCCCCCCCCCCC] |
| 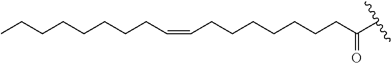 [SMILES: *C(=O)CCCCCCC/C=C\CCCCCCCC] | 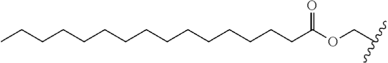 [SMILES: *COC(=O)CCCCCCCCCCCCCCC] |
| 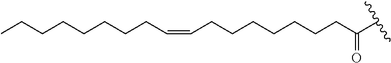 [SMILES: *C(=O)CCCCCCC/C=C\CCCCCCCC] | 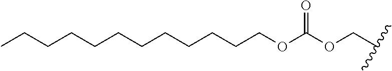 [SMILES: *COC(=O)OCCCCCCCCCCCC] |
| 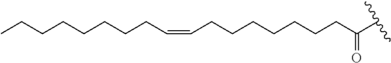 [SMILES: *C(=O)CCCCCCC/C=C\CCCCCCCC] | 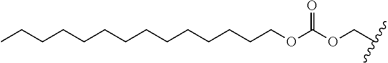 [SMILES: *COC(=O)OCCCCCCCCCCCCCC] |
| 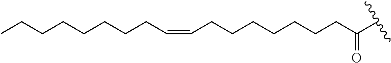 [SMILES: *C(=O)CCCCCCC/C=C\CCCCCCCC] | 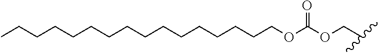 [SMILES: *COC(=O)OCCCCCCCCCCCCCCCC] |
| 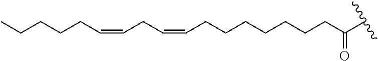 [SMILES: *C(=O)CCCCCCC/C=C\C/C=C\CCCCC] | 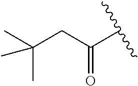 [SMILES: *C(=O)CC(C)(C)C] |
| 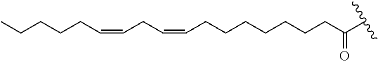 [SMILES: *C(=O)CCCCCCC/C=C\C/C=C\CCCCC] | 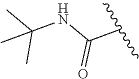 [SMILES: *C(=O)NC(C)(C)C] |
| 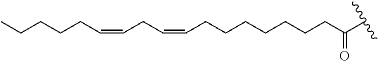 [SMILES: *C(=O)CCCCCCC/C=C\C/C=C\CCCCC] | 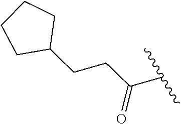 [SMILES: *C(=O)CCC1CCCC1] |
| 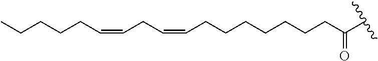 [SMILES: *C(=O)CCCCCCC/C=C\C/C=C\CCCCC] | 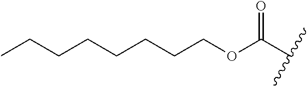 [SMILES: *C(=O)OCCCCCCCC] |
| 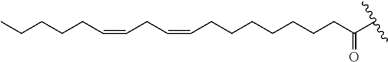 [SMILES: *C(=O)CCCCCCC/C=C\C/C=C\CCCCC] | 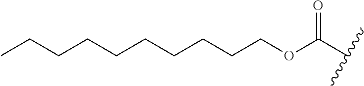 [SMILES: *C(=O)OCCCCCCCCCC] |
| 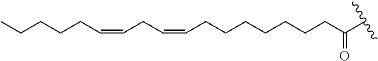 [SMILES: *C(=O)CCCCCCC/C=C\C/C=C\CCCCC] | 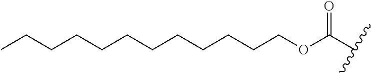 [SMILES: *C(=O)OCCCCCCCCCCCC] |
| 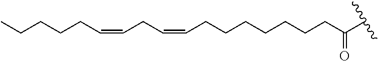 [SMILES: *C(=O)CCCCCCC/C=C\C/C=C\CCCCC] | 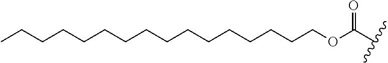 [SMILES: *C(=O)OCCCCCCCCCCCCCCCC] |
| 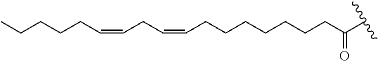 [SMILES: *C(=O)CCCCCCC/C=C\C/C=C\CCCCC] | 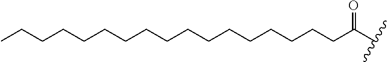 [SMILES: *C(=O)CCCCCCCCCCCCCCCCC] |
| 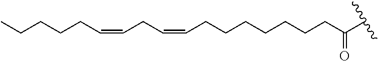 [SMILES: *C(=O)CCCCCCC/C=C\C/C=C\CCCCC] | 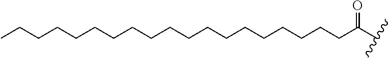 [SMILES: *C(=O)CCCCCCCCCCCCCCCCCCC] |
| 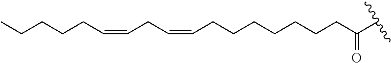 [SMILES: *C(=O)CCCCCCC/C=C\C/C=C\CCCCC] | 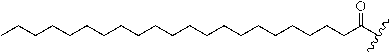 [SMILES: *C(=O)CCCCCCCCCCCCCCCCCCCCC] |
| 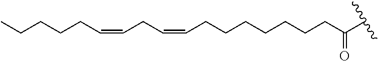 [SMILES: *C(=O)CCCCCCC/C=C\C/C=C\CCCCC] | 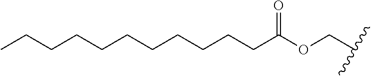 [SMILES: *COC(=O)CCCCCCCCCCC] |
| 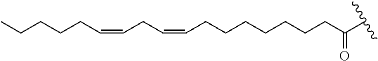 [SMILES: *C(=O)CCCCCCC/C=C\C/C=C\CCCCC] | 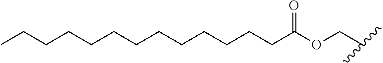 [SMILES: *COC(=O)CCCCCCCCCCCCC] |
| 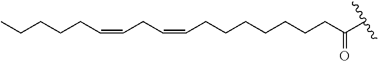 [SMILES: *C(=O)CCCCCCC/C=C\C/C=C\CCCCC] | 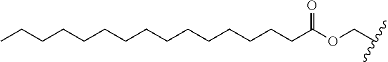 [SMILES: *COC(=O)CCCCCCCCCCCCCCC] |
| 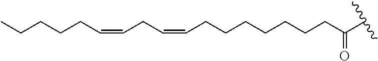 [SMILES: *C(=O)CCCCCCC/C=C\C/C=C\CCCCC] | 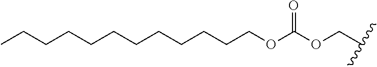 [SMILES: *COC(=O)OCCCCCCCCCCCC] |
| 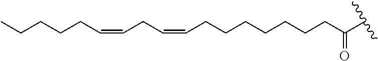 [SMILES: *C(=O)CCCCCCC/C=C\C/C=C\CCCCC] | 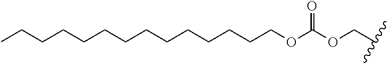 [SMILES: *COC(=O)OCCCCCCCCCCCCCC] |
| 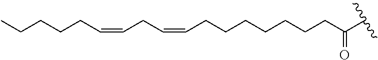 [SMILES: *C(=O)CCCCCCC/C=C\C/C=C\CCCCC] | 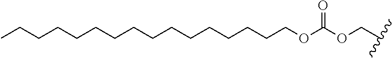 [SMILES: *COC(=O)OCCCCCCCCCCCCCCCC] |
| 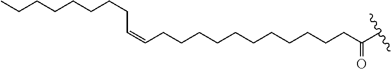 [SMILES: *C(=O)CCCCCCCCCCC/C=C\CCCCCCCC] | 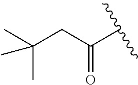 [SMILES: *C(=O)CC(C)(C)C] |
| 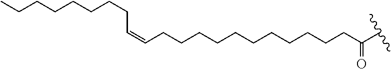 [SMILES: *C(=O)CCCCCCCCCCC/C=C\CCCCCCCC] | 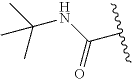 [SMILES: *C(=O)NC(C)(C)C] |
| 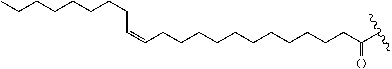 [SMILES: *C(=O)CCCCCCCCCCC/C=C\CCCCCCCC] | 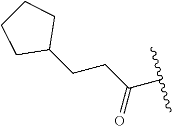 [SMILES: *C(=O)CCC1CCCC1] |
| 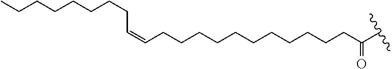 [SMILES: *C(=O)CCCCCCCCCCC/C=C\CCCCCCCC] | 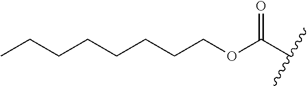 [SMILES: *C(=O)OCCCCCCCC] |
| 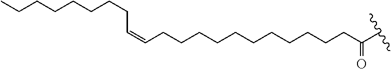 [SMILES: *C(=O)CCCCCCCCCCC/C=C\CCCCCCCC] | 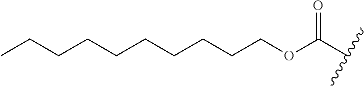 [SMILES: *C(=O)OCCCCCCCCCC] |
| 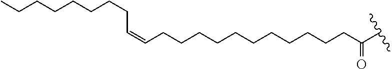 [SMILES: *C(=O)CCCCCCCCCCC/C=C\CCCCCCCC] | 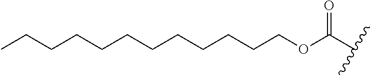 [SMILES: *C(=O)OCCCCCCCCCCCC] |
| 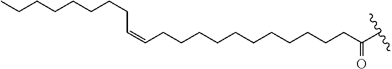 [SMILES: *C(=O)CCCCCCCCCCC/C=C\CCCCCCCC] | 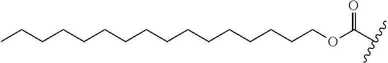 [SMILES: *C(=O)OCCCCCCCCCCCCCCCC] |
| 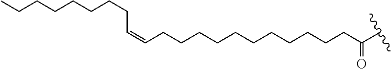 [SMILES: *C(=O)CCCCCCCCCCC/C=C\CCCCCCCC] | 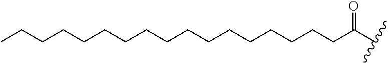 [SMILES: *C(=O)CCCCCCCCCCCCCCCCC] |
| 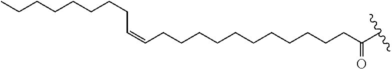 [SMILES: *C(=O)CCCCCCCCCCC/C=C\CCCCCCCC] | 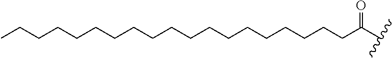 [SMILES: *C(=O)CCCCCCCCCCCCCCCCCCC] |
| 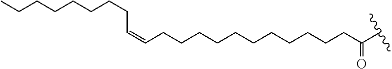 [SMILES: *C(=O)CCCCCCCCCCC/C=C\CCCCCCCC] | 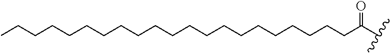 [SMILES: *C(=O)CCCCCCCCCCCCCCCCCCCCC] |
| 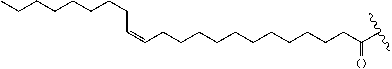 [SMILES: *C(=O)CCCCCCCCCCC/C=C\CCCCCCCC] | 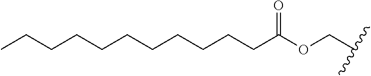 [SMILES: *COC(=O)CCCCCCCCCCC] |
| 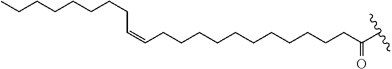 [SMILES: *C(=O)CCCCCCCCCCC/C=C\CCCCCCCC] | 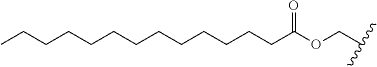 [SMILES: *COC(=O)CCCCCCCCCCCCC] |
| 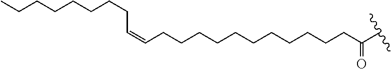 [SMILES: *C(=O)CCCCCCCCCCC/C=C\CCCCCCCC] | 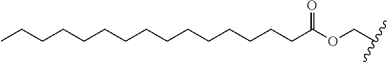 [SMILES: *COC(=O)CCCCCCCCCCCCCCC] |
| 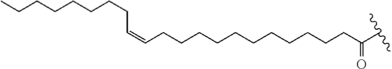 [SMILES: *C(=O)CCCCCCCCCCC/C=C\CCCCCCCC] | 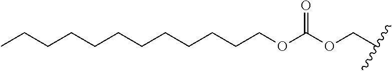 [SMILES: *COC(=O)OCCCCCCCCCCCC] |
| 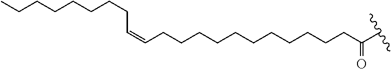 [SMILES: *C(=O)CCCCCCCCCCC/C=C\CCCCCCCC] | 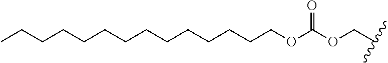 [SMILES: *COC(=O)OCCCCCCCCCCCCCC] |
| 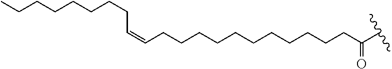 [SMILES: *C(=O)CCCCCCCCCCC/C=C\CCCCCCCC] | 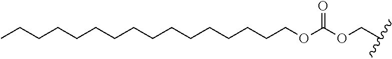 [SMILES: *COC(=O)OCCCCCCCCCCCCCCCC] |
| 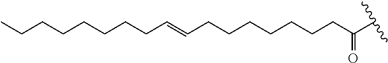 [SMILES: *C(=O)CCCCCCC/C=C/CCCCCCCC] | 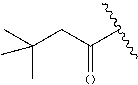 [SMILES: *C(=O)CC(C)(C)C] |
| 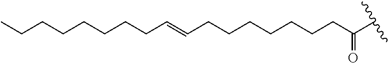 [SMILES: *C(=O)CCCCCCC/C=C/CCCCCCCC] | 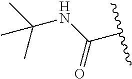 [SMILES: *C(=O)NC(C)(C)C] |
| 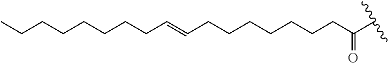 [SMILES: *C(=O)CCCCCCC/C=C/CCCCCCCC] | 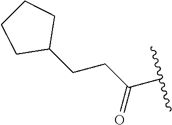 [SMILES: *C(=O)CCC1CCCC1] |
| 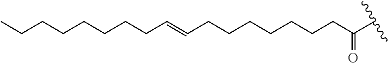 [SMILES: *C(=O)CCCCCCC/C=C/CCCCCCCC] | 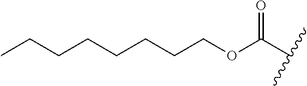 [SMILES: *C(=O)OCCCCCCCC] |
| 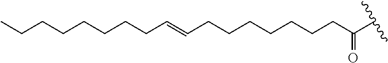 [SMILES: *C(=O)CCCCCCC/C=C/CCCCCCCC] | 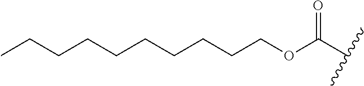 [SMILES: *C(=O)OCCCCCCCCCC] |
| 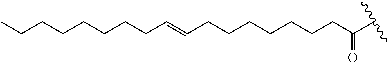 [SMILES: *C(=O)CCCCCCC/C=C/CCCCCCCC] | 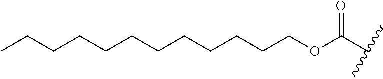 [SMILES: *C(=O)OCCCCCCCCCCCC] |
| 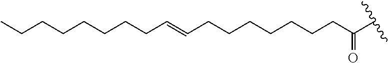 [SMILES: *C(=O)CCCCCCC/C=C/CCCCCCCC] | 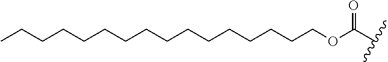 [SMILES: *C(=O)OCCCCCCCCCCCCCCCC] |
| 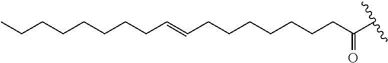 [SMILES: *C(=O)CCCCCCC/C=C/CCCCCCCC] | 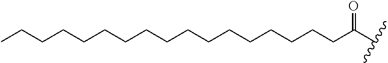 [SMILES: *C(=O)CCCCCCCCCCCCCCCCC] |
| 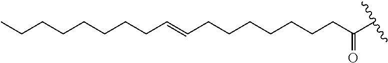 [SMILES: *C(=O)CCCCCCC/C=C/CCCCCCCC] | 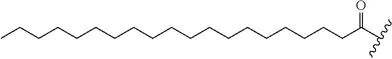 [SMILES: *C(=O)CCCCCCCCCCCCCCCCCCC] |
| 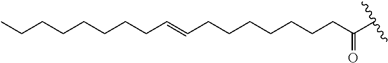 [SMILES: *C(=O)CCCCCCC/C=C/CCCCCCCC] | 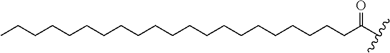 [SMILES: *C(=O)CCCCCCCCCCCCCCCCCCCCC] |
| 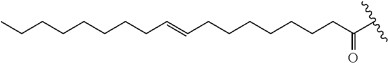 [SMILES: *C(=O)CCCCCCC/C=C/CCCCCCCC] | 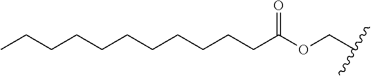 [SMILES: *COC(=O)CCCCCCCCCCC] |
| 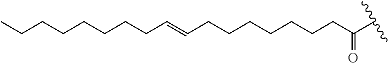 [SMILES: *C(=O)CCCCCCC/C=C/CCCCCCCC] | 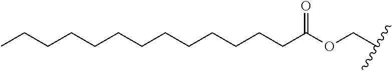 [SMILES: *COC(=O)CCCCCCCCCCCCC] |
| 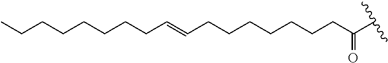 [SMILES: *C(=O)CCCCCCC/C=C/CCCCCCCC] | 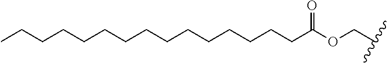 [SMILES: *COC(=O)CCCCCCCCCCCCCCC] |
| 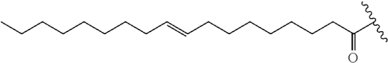 [SMILES: *C(=O)CCCCCCC/C=C/CCCCCCCC] | 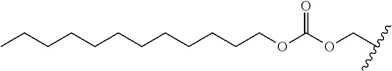 [SMILES: *COC(=O)OCCCCCCCCCCCC] |
| 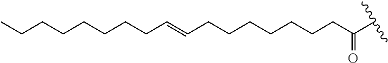 [SMILES: *C(=O)CCCCCCC/C=C/CCCCCCCC] | 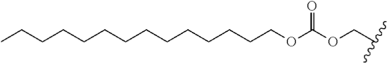 [SMILES: *COC(=O)OCCCCCCCCCCCCCC] |
| 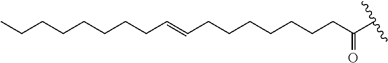 [SMILES: *C(=O)CCCCCCC/C=C/CCCCCCCC] | 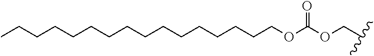 [SMILES: *COC(=O)OCCCCCCCCCCCCCCCC] |
| 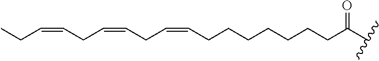 [SMILES: *C(=O)CCCCCCC/C=C\C/C=C\C/C=C\CC] | 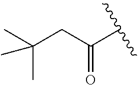 [SMILES: *C(=O)CC(C)(C)C] |
| 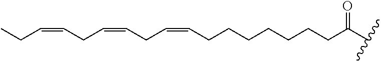 [SMILES: *C(=O)CCCCCCC/C=C\C/C=C\C/C=C\CC] | 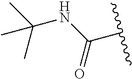 [SMILES: *C(=O)NC(C)(C)C] |
| 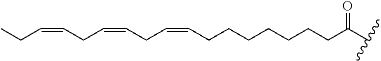 [SMILES: *C(=O)CCCCCCC/C=C\C/C=C\C/C=C\CC] | 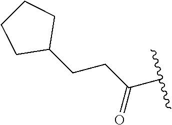 [SMILES: *C(=O)CCC1CCCC1] |
| 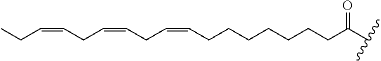 [SMILES: *C(=O)CCCCCCC/C=C\C/C=C\C/C=C\CC] | 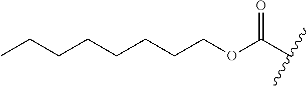 [SMILES: *C(=O)OCCCCCCCC] |
| 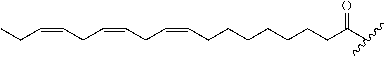 [SMILES: *C(=O)CCCCCCC/C=C\C/C=C\C/C=C\CC] | 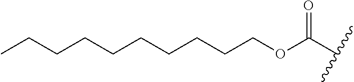 [SMILES: *C(=O)OCCCCCCCCCC] |
| 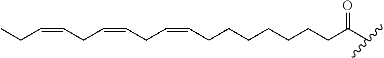 [SMILES: *C(=O)CCCCCCC/C=C\C/C=C\C/C=C\CC] | 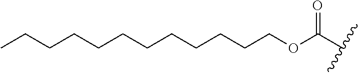 [SMILES: *C(=O)OCCCCCCCCCCCC] |
| 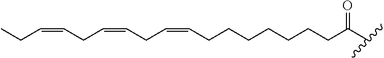 [SMILES: *C(=O)CCCCCCC/C=C\C/C=C\C/C=C\CC] | 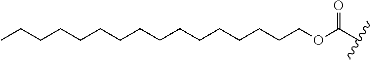 [SMILES: *C(=O)OCCCCCCCCCCCCCCCC] |
| 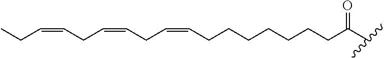 [SMILES: *C(=O)CCCCCCC/C=C\C/C=C\C/C=C\CC] | 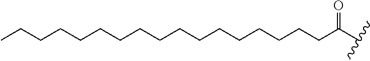 [SMILES: *C(=O)CCCCCCCCCCCCCCCCC] |
| 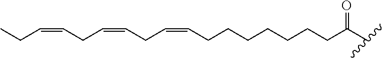 [SMILES: *C(=O)CCCCCCC/C=C\C/C=C\C/C=C\CC] | 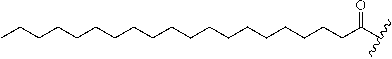 [SMILES: *C(=O)CCCCCCCCCCCCCCCCCCC] |
| 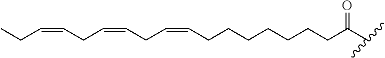 [SMILES: *C(=O)CCCCCCC/C=C\C/C=C\C/C=C\CC] | 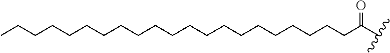 [SMILES: *C(=O)CCCCCCCCCCCCCCCCCCCCC] |
| 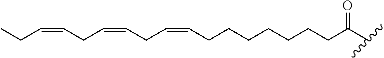 [SMILES: *C(=O)CCCCCCC/C=C\C/C=C\C/C=C\CC] | 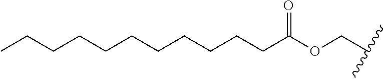 [SMILES: *COC(=O)CCCCCCCCCCC] |
| 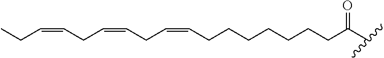 [SMILES: *C(=O)CCCCCCC/C=C\C/C=C\C/C=C\CC] | 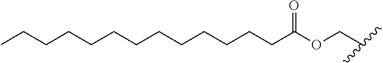 [SMILES: *COC(=O)CCCCCCCCCCCCC] |
| 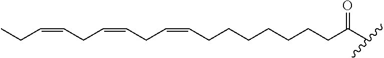 [SMILES: *C(=O)CCCCCCC/C=C\C/C=C\C/C=C\CC] | 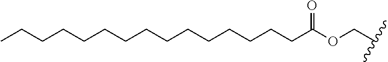 [SMILES: *COC(=O)CCCCCCCCCCCCCCC] |
| 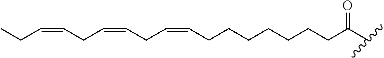 [SMILES: *C(=O)CCCCCCC/C=C\C/C=C\C/C=C\CC] | 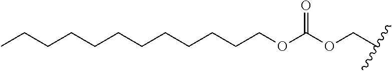 [SMILES: *COC(=O)OCCCCCCCCCCCC] |
| 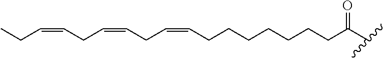 [SMILES: *C(=O)CCCCCCC/C=C\C/C=C\C/C=C\CC] | 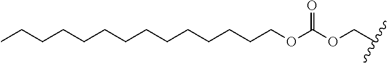 [SMILES: *COC(=O)OCCCCCCCCCCCCCC] |
| 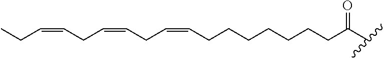 [SMILES: *C(=O)CCCCCCC/C=C\C/C=C\C/C=C\CC] | 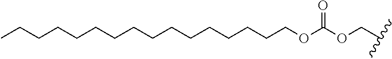 [SMILES: *COC(=O)OCCCCCCCCCCCCCCCC] |
| tBu | 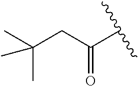 [SMILES: *C(=O)CC(C)(C)C] |
| tBu | 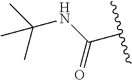 [SMILES: *C(=O)NC(C)(C)C] |
| tBu | 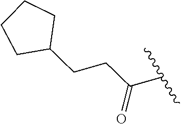 [SMILES: *C(=O)CCC1CCCC1] |
| tBu | 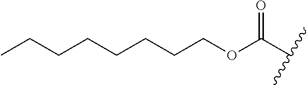 [SMILES: *C(=O)OCCCCCCCC] |
| tBu | 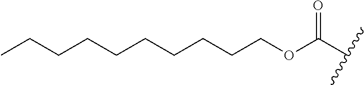 [SMILES: *C(=O)OCCCCCCCCCC] |
| tBu | 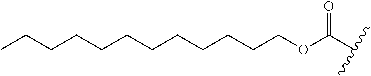 [SMILES: *C(=O)OCCCCCCCCCCCC] |
| tBu | 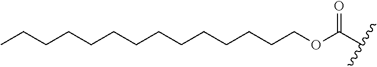 [SMILES: *C(=O)OCCCCCCCCCCCCCC] |
| tBu | 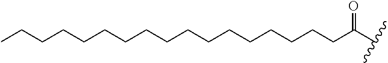 [SMILES: *C(=O)CCCCCCCCCCCCCCCCC] |
| tBu | 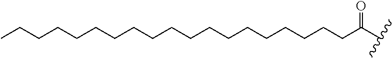 [SMILES: *C(=O)CCCCCCCCCCCCCCCCCCC] |
| tBu | 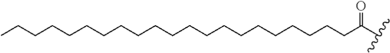 [SMILES: *C(=O)CCCCCCCCCCCCCCCCCCCCC] |
| tBu | 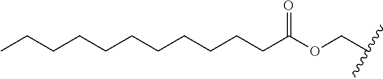 [SMILES: *COC(=O)CCCCCCCCCCC] |
| tBu | 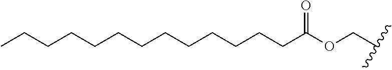 [SMILES: *COC(=O)CCCCCCCCCCCCC] |
| tBu | 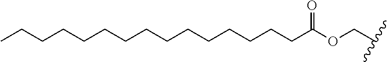 [SMILES: *COC(=O)CCCCCCCCCCCCCCC] |
| tBu | 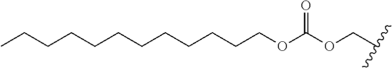 [SMILES: *COC(=O)OCCCCCCCCCCCC] |
| tBu | 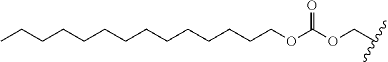 [SMILES: *COC(=O)OCCCCCCCCCCCCCC] |
| tBu | 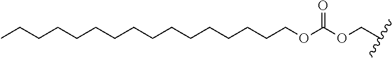 [SMILES: *COC(=O)OCCCCCCCCCCCCCCCC] |
| phenyl | 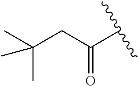 [SMILES: *C(=O)CC(C)(C)C] |
| phenyl | 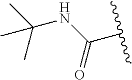 [SMILES: *C(=O)NC(C)(C)C] |
| phenyl | 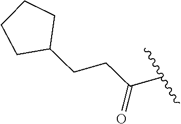 [SMILES: *C(=O)CCC1CCCC1] |
| phenyl | 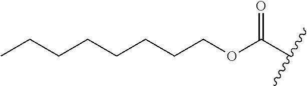 [SMILES: *C(=O)OCCCCCCCC] |
| phenyl | 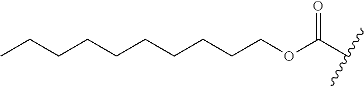 [SMILES: *C(=O)OCCCCCCCCCC] |
| phenyl | 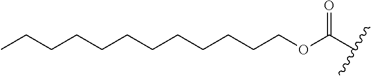 [SMILES: *C(=O)OCCCCCCCCCCCC] |
| phenyl | 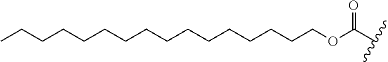 [SMILES: *C(=O)OCCCCCCCCCCCCCCCC] |
| phenyl | 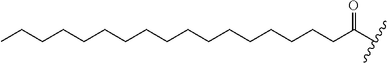 [SMILES: *C(=O)CCCCCCCCCCCCCCCCC] |
| phenyl | 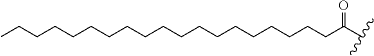 [SMILES: *C(=O)CCCCCCCCCCCCCCCCCCC] |
| phenyl | 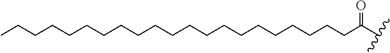 [SMILES: *C(=O)CCCCCCCCCCCCCCCCCCCCC] |
| phenyl | 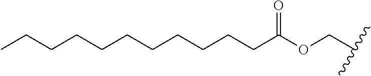 [SMILES: *COC(=O)CCCCCCCCCCC] |
| phenyl | 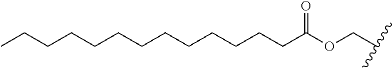 [SMILES: *COC(=O)CCCCCCCCCCCCC] |
| phenyl | 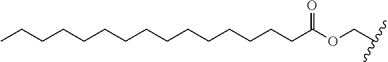 [SMILES: *COC(=O)CCCCCCCCCCCCCCC] |
| phenyl | 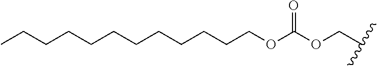 [SMILES: *COC(=O)OCCCCCCCCCCCC] |
| phenyl | 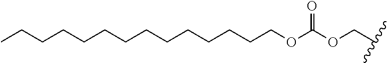 [SMILES: *COC(=O)OCCCCCCCCCCCCCC] |
| phenyl | 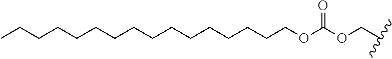 [SMILES: *COC(=O)OCCCCCCCCCCCCCCCC] |
| 4-chlorophenyl | 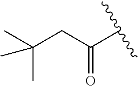 [SMILES: *C(=O)CC(C)(C)C] |
| 4-chlorophenyl | 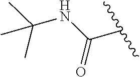 [SMILES: *C(=O)NC(C)(C)C] |
| 4-chlorophenyl | 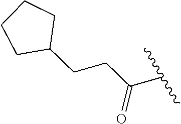 [SMILES: *C(=O)CCC1CCCC1] |
| 4-chlorophenyl | 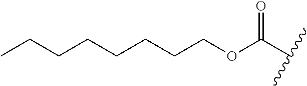 [SMILES: *C(=O)OCCCCCCCC] |
| 4-chlorophenyl | 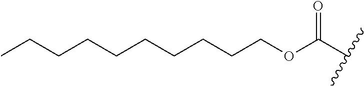 [SMILES: *C(=O)OCCCCCCCCCC] |
| 4-chlorophenyl | 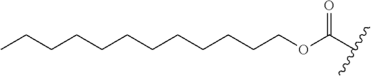 [SMILES: *C(=O)OCCCCCCCCCCCC] |
| 4-chlorophenyl | 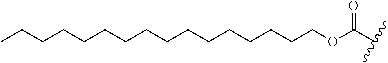 [SMILES: *C(=O)OCCCCCCCCCCCCCCCC] |
| 4-chlorophenyl | 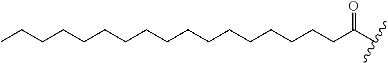 [SMILES: *C(=O)CCCCCCCCCCCCCCCCC] |
| 4-chlorophenyl | 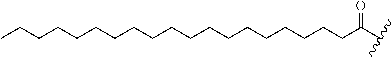 [SMILES: *C(=O)CCCCCCCCCCCCCCCCCCC] |
| 4-chlorophenyl | 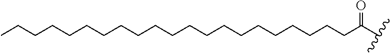 [SMILES: *C(=O)CCCCCCCCCCCCCCCCCCCCC] |
| 4-chlorophenyl | 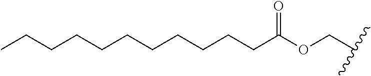 [SMILES: *COC(=O)CCCCCCCCCCC] |
| 4-chlorophenyl | 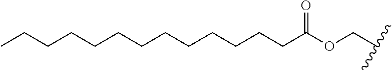 [SMILES: *COC(=O)CCCCCCCCCCCCC] |
| 4-chlorophenyl | 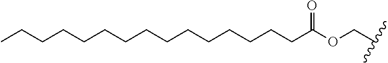 [SMILES: *COC(=O)CCCCCCCCCCCCCCC] |
| 4-chlorophenyl | 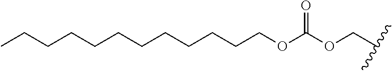 [SMILES: *COC(=O)OCCCCCCCCCCCC] |
| 4-chlorophenyl | 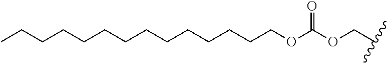 [SMILES: *COC(=O)OCCCCCCCCCCCCCC] |
| 4-chlorophenyl | 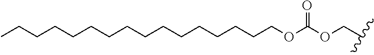 [SMILES: *COC(=O)OCCCCCCCCCCCCCCCC] |
| 3,4-dichlorophenyl | 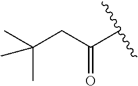 [SMILES: *C(=O)CC(C)(C)C] |
| 3,4-dichlorophenyl | 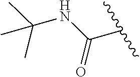 [SMILES: *C(=O)NC(C)(C)C] |
| 3,4-dichlorophenyl | 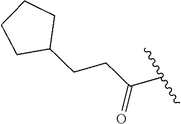 [SMILES: *C(=O)CCC1CCCC1] |
| 3,4-dichlorophenyl | 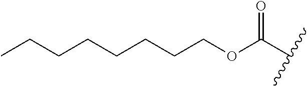 [SMILES: *C(=O)OCCCCCCCC] |
| 3,4-dichlorophenyl | 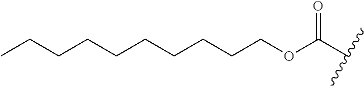 [SMILES: *C(=O)OCCCCCCCCCC] |
| 3,4-dichlorophenyl | 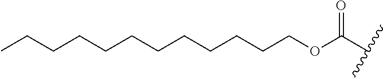 [SMILES: *C(=O)OCCCCCCCCCCCC] |
| 3,4-dichlorophenyl | 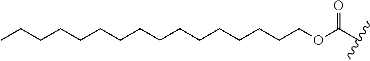 [SMILES: *C(=O)OCCCCCCCCCCCCCCCC] |
| 3,4-dichlorophenyl | 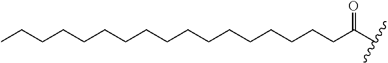 [SMILES: *C(=O)CCCCCCCCCCCCCCCCC] |
| 3,4-dichlorophenyl | 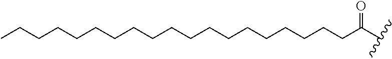 [SMILES: *C(=O)CCCCCCCCCCCCCCCCCCC] |
| 3,4-dichlorophenyl | 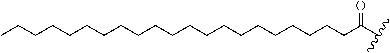 [SMILES: *C(=O)CCCCCCCCCCCCCCCCCCCCC] |
| 3,4-dichlorophenyl | 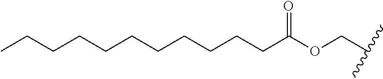 [SMILES: *COC(=O)CCCCCCCCCCC] |
| 3,4-dichlorophenyl | 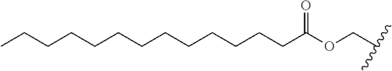 [SMILES: *COC(=O)CCCCCCCCCCCCC] |
| 3,4-dichlorophenyl | 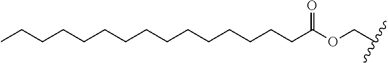 [SMILES: *COC(=O)CCCCCCCCCCCCCCC] |
| 3,4-dichlorophenyl | 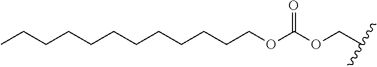 [SMILES: *COC(=O)OCCCCCCCCCCCC] |
| 3,4-dichlorophenyl | 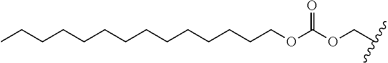 [SMILES: *COC(=O)OCCCCCCCCCCCCCC] |
| 3,4-dichlorophenyl | 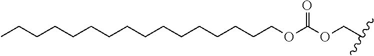 [SMILES: *COC(=O)OCCCCCCCCCCCCCCCC] |
| 4-methylphenyl | 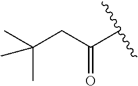 [SMILES: *C(=O)CC(C)(C)C] |
| 4-methylphenyl | 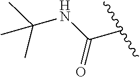 [SMILES: *C(=O)NC(C)(C)C] |
| 4-methylphenyl | 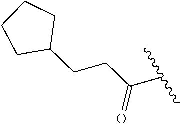 [SMILES: *C(=O)CCC1CCCC1] |
| 4-methylphenyl | 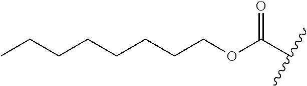 [SMILES: *C(=O)OCCCCCCCC] |
| 4-methylphenyl | 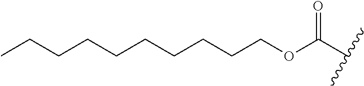 [SMILES: *C(=O)OCCCCCCCCCC] |
| 4-methylphenyl | 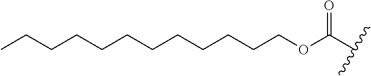 [SMILES: *C(=O)OCCCCCCCCCCCC] |
| 4-methylphenyl | 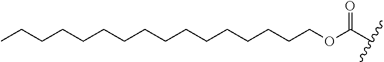 [SMILES: *C(=O)OCCCCCCCCCCCCCCCC] |
| 4-methylphenyl | 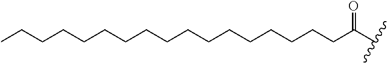 [SMILES: *C(=O)CCCCCCCCCCCCCCCCC] |
| 4-methylphenyl | 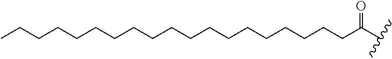 [SMILES: *C(=O)CCCCCCCCCCCCCCCCCCC] |
| 4-methylphenyl | 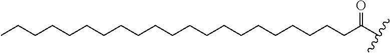 [SMILES: *C(=O)CCCCCCCCCCCCCCCCCCCCC] |
| 4-methylphenyl | 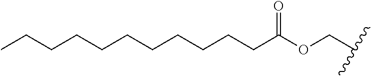 [SMILES: *COC(=O)CCCCCCCCCCC] |
| 4-methylphenyl | 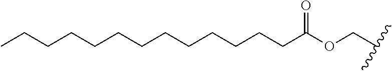 [SMILES: *COC(=O)CCCCCCCCCCCCC] |
| 4-methylphenyl | 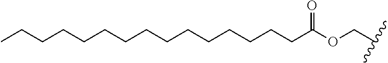 [SMILES: *COC(=O)CCCCCCCCCCCCCCC] |
| 4-methylphenyl | 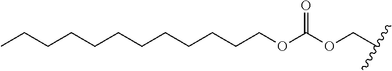 [SMILES: *COC(=O)OCCCCCCCCCCCC] |
| 4-methylphenyl | 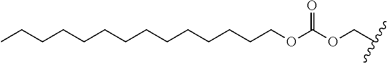 [SMILES: *COC(=O)OCCCCCCCCCCCCCC] |
| 4-methylphenyl | 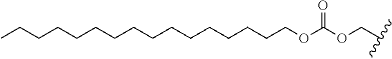 [SMILES: *COC(=O)OCCCCCCCCCCCCCCCC] |
| 4-methoxyphenyl | 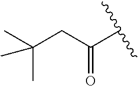 [SMILES: *C(=O)CC(C)(C)C] |
| 4-methoxyphenyl | 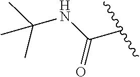 [SMILES: *C(=O)NC(C)(C)C] |
| 4-methoxyphenyl | 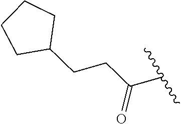 [SMILES: *C(=O)CCC1CCCC1] |
| 4-methoxyphenyl | 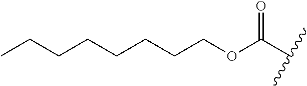 [SMILES: *C(=O)OCCCCCCCC] |
| 4-methoxyphenyl | 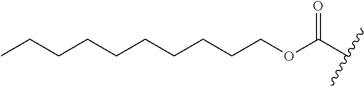 [SMILES: *C(=O)OCCCCCCCCCC] |
| 4-methoxyphenyl | 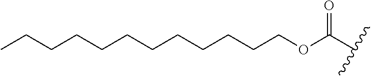 [SMILES: *C(=O)OCCCCCCCCCCCC] |
| 4-methoxyphenyl | 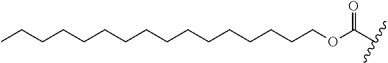 [SMILES: *C(=O)OCCCCCCCCCCCCCCCC] |
| 4-methoxyphenyl | 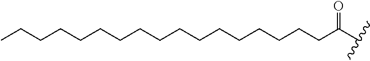 [SMILES: *C(=O)CCCCCCCCCCCCCCCCC] |
| 4-methoxyphenyl | 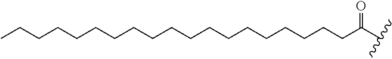 [SMILES: *C(=O)CCCCCCCCCCCCCCCCCCC] |
| 4-methoxyphenyl | 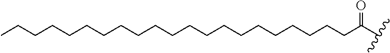 [SMILES: *C(=O)CCCCCCCCCCCCCCCCCCCCC] |
| 4-methoxyphenyl | 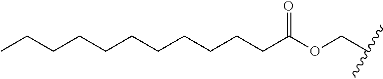 [SMILES: *COC(=O)CCCCCCCCCCC] |
| 4-methoxyphenyl | 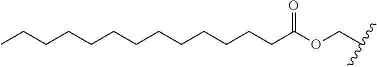 [SMILES: *COC(=O)CCCCCCCCCCCCC] |
| 4-methoxyphenyl | 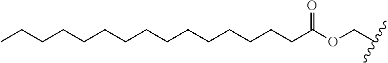 [SMILES: *COC(=O)CCCCCCCCCCCCCCC] |
| 4-methoxyphenyl | 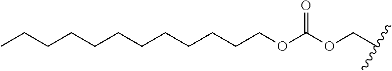 [SMILES: *COC(=O)OCCCCCCCCCCCC] |
| 4-methoxyphenyl | 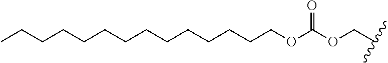 [SMILES: *COC(=O)OCCCCCCCCCCCCCC] |
| 4-methoxyphenyl | 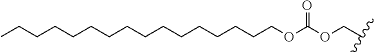 [SMILES: *COC(=O)OCCCCCCCCCCCCCCCC] |

In a 61^{st} aspect provided herein is a compound, or pharmaceutically acceptable salt thereof, having a structure provided in Formula (I-20),

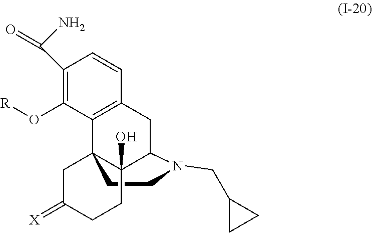

wherein X is ═O, R is R⁴OP(OCH₃)(O)—O—CH(R³)— and R³ and R⁴ are any of the pair of R³/R⁴ groups recited in Table 10. Alternatively, X is ═CH₂, R is
R⁴OP(OCH₃)(O)—O—CH(R³)— and R³ and R⁴ are any of the pair of R³/R⁴ groups recited in Table 10.

In a 62^{nd} aspect, provided herein is a compound, or pharmaceutically acceptable salt thereof, having a structure provided in Formula (II-20),

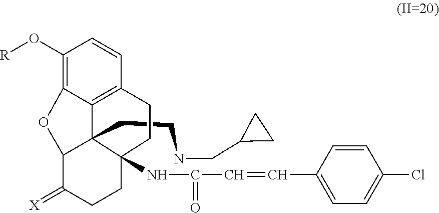

wherein X is ═O, R is R⁴OP(OCH)(O)—O—CH(R³)— and R³ and R⁴ are any of the pair of R³/R⁴ groups recited in Table 10. Alternatively, X is ═CH₂, R is
R⁴OP(OCH₃)(O)—O—CH(R³)— and R³ and R⁴ are any of the pair of R³/R⁴ groups recited in Table 10.

In a 63^{rd} aspect, provided herein is a compound, or pharmaceutically acceptable salt thereof, having a structure provided in Formula (III-20),

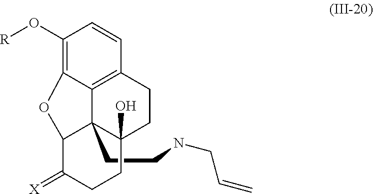

wherein X is ═O, R is R⁴OP(OCH)(O)—O—CH(R³)— and R³ and R⁴ are any of the pair of R³/R⁴ groups recited in Table 10. Alternatively, X is ═CH₂, R is
R⁴OP(OCH₃)(O)—O—CH(R³)— and R³ and R⁴ are any of the pair of R³/R⁴ groups recited in Table 10.

TABLE 10

| R³ | R⁴ |
|---|---|
| H | Me |
| H | —C₇ H₁₅ |
| H | —C₉H₁₉ |
| H | —C₁₁H₂₃ |
| H | —C₁₃H₂₇ |
| H | —C₁₅H₃₁ |
| H | —C₁₇H₃₅ |
| H | 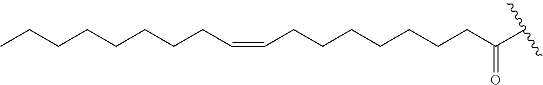 [SMILES: *C(=O)CCCCCCC/C=C\CCCCCCCC] |
| H | 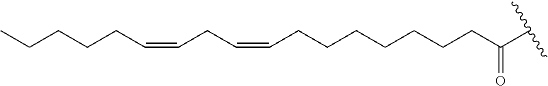 [SMILES: *C(=O)CCCCCCC/C=C\C/C=C\CCCCC] |
| H | 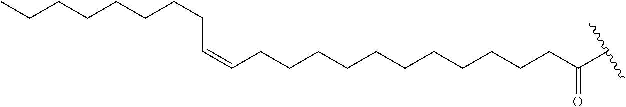 [SMILES: *C(=O)CCCCCCCCCCC/C=C\CCCCCCCC] |
| H | 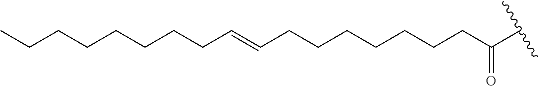 [SMILES: *C(=O)CCCCCCC/C=C/CCCCCCCC] |
| H | 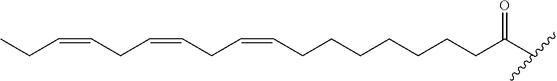 [SMILES: *C(=O)CCCCCCC/C=C\C/C=C\C/C=C\CC] |
| H | tBu |
| H | phenyl |
| H | 4-chlorophenyl |
| H | 3,4-dichlorophenyl |
| H | 4-methylphenyl |
| H | 4-methoxyphenyl |
| Me | Me |
| Me | —C₇H₁₅ |
| Me | —C₉H₁₉ |
| Me | —C₁₁H₂₃ |
| Me | —C₁₃H₂₇ |
| Me | —C₁₅H₃₁ |
| Me | —C₁₇H₃₅ |
| Me | 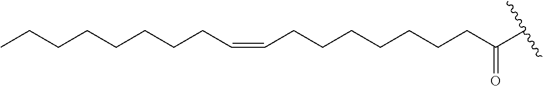 [SMILES: *C(=O)CCCCCCC/C=C\CCCCCCCC] |
| Me | 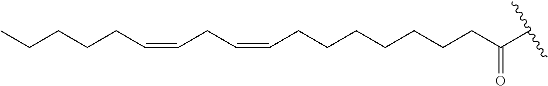 [SMILES: *C(=O)CCCCCCC/C=C\C/C=C\CCCCC] |
| Me | 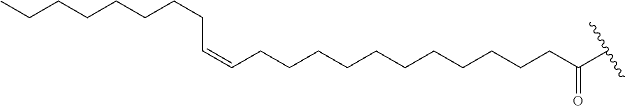 [SMILES: *C(=O)CCCCCCCCCCC/C=C\CCCCCCCC] |
| Me | 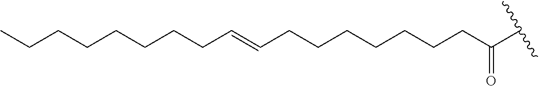 [SMILES: *C(=O)CCCCCCC/C=C/CCCCCCCC] |
| Me | 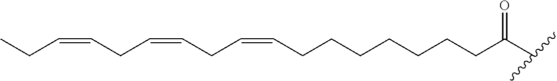 [SMILES: *C(=O)CCCCCCC/C=C\C/C=C\C/C=C\CC] |
| Me | tBu |
| Me | phenyl |
| Me | 4-chlorophenyl |
| Me | 3,4-dichlorophenyl |
| Me | 4-methylphenyl |
| Me | 4-methoxyphenyl |
| tBu | Me |
| tBu | —C₇ H₁₅ |
| tBu | —C₉H₁₉ |
| tBu | —C₁₁H₂₃ |
| tBu | —C₁₃H₂₇ |
| tBu | —C₁₅H₃₁ |
| tBu | —C₁₇H₃₅ |
| tBu | 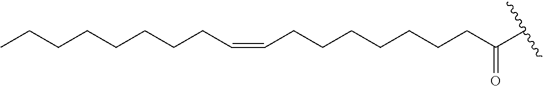 [SMILES: *C(=O)CCCCCCC/C=C\CCCCCCCC] |
| tBu | 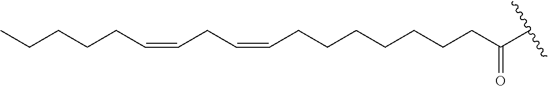 [SMILES: *C(=O)CCCCCCC/C=C\C/C=C\CCCCC] |
| tBu | 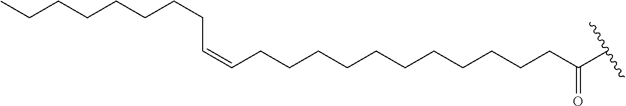 [SMILES: *C(=O)CCCCCCCCCCC/C=C\CCCCCCCC] |
| tBu | 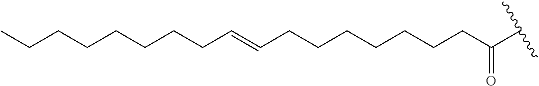 [SMILES: *C(=O)CCCCCCC/C=C/CCCCCCCC] |
| tBu | 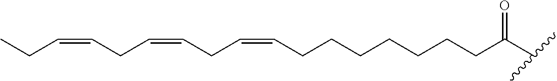 [SMILES: *C(=O)CCCCCCC/C=C\C/C=C\C/C=C\CC] |
| tBu | phenyl |
| tBu | tBu |
| tBu | 4-chlorophenyl |
| tBu | 3,4-dichlorophenyl |
| tBu | 4-methylphenyl |
| tBu | 4-methoxyphenyl |
| —C₇H₁₅ | Me |
| —C₇H₁₅ | —C₇H₁₅ |
| —C₇H₁₅ | —C₉H₁₉ |
| —C₇H₁₅ | —C₁₁H₂₃ |
| —C₇H₁₅ | —C₁₃H₂₇ |
| —C₇H₁₅ | —C₁₅H₃₁ |
| —C₇H₁₅ | —CH₁₇H₃₅ |
| —C₇H₁₅ | 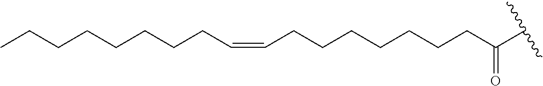 [SMILES: *C(=O)CCCCCCC/C=C\CCCCCCCC] |
| —C₇H₁₅ | 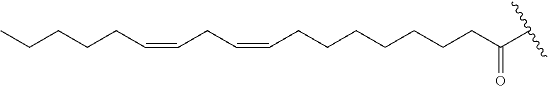 [SMILES: *C(=O)CCCCCCC/C=C\C/C=C\CCCCC] |
| —C₇H₁₅ | 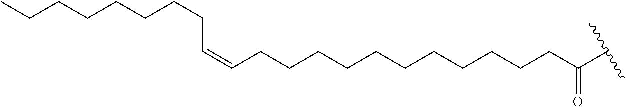 [SMILES: *C(=O)CCCCCCCCCCC/C=C\CCCCCCCC] |
| —C₇H₁₅ | 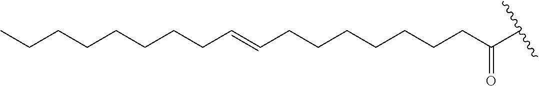 [SMILES: *C(=O)CCCCCCC/C=C/CCCCCCCC] |
| —C₇H₁₅ | 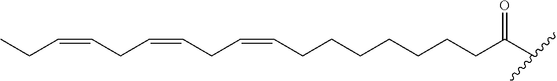 [SMILES: *C(=O)CCCCCCC/C=C\C/C=C\C/C=C\CC] |
| —C₇H₁₅ | tBu |
| —C₇H₁₅ | phenyl |
| —C₇H₁₅ | 4-chlorophenyl |
| —C₇H₁₅ | 3,4-dichlorophenyl |
| —C₇H₁₅ | 4-methylphenyl |
| —C₇H₁₅ | 4-methoxyphenyl |
| —C₉ H₁₉ | Me |
| —C₉H₁₉ | —C₇H₁₅ |
| —C₉H₁₉ | —C₉H₁₉ |
| —C₉H₁₉ | —C₁₁H₂₃ |
| —C₉H₁₉ | —C₁₃H₂₇ |
| —C₉H₁₉ | —C₁₅H₃₁ |
| —C₉H₁₉ | —C₁₇H₃₅ |
| —C₉H₁₉ | 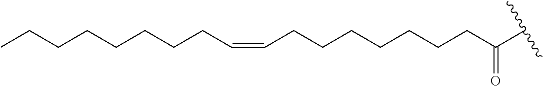 [SMILES: *C(=O)CCCCCCC/C=C\CCCCCCCC] |
| —C₉H₁₉ | 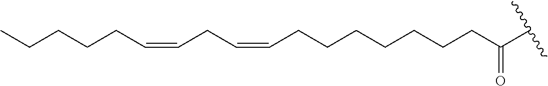 [SMILES: *C(=O)CCCCCCC/C=C\C/C=C\CCCCC] |
| —C₉H₁₉ | 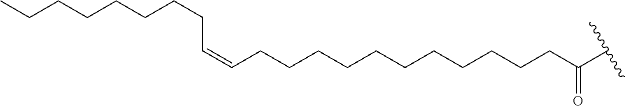 [SMILES: *C(=O)CCCCCCCCCCC/C=C\CCCCCCCC] |
| —C₉H₁₉ | 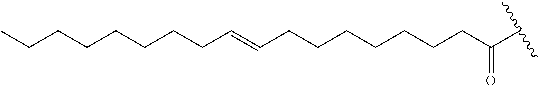 [SMILES: *C(=O)CCCCCCC/C=C/CCCCCCCC] |
| —C₉H₁₉ | 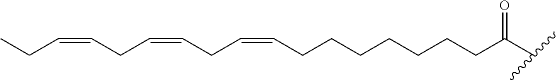 [SMILES: *C(=O)CCCCCCC/C=C\C/C=C\C/C=C\CC] |
| —C₉H₁₉ | tBu |
| —C₉H₁₉ | phenyl |
| —C₉H₁₉ | 4-chlorophenyl |
| —C₉H₁₉ | 3,4-dichlorophenyl |
| —C₉H₁₉ | 4-methylphenyl |
| —C₉H₁₉ | 4-methoxyphenyl |
| —C₁₁H₂₃ | Me |
| —C₁₁H₂₃ | —C₇H₁₅ |
| —C₁₁H₂₃ | —C₉H₁₉ |
| —C₁₁H₂₃ | —C₁₁H₂₃ |
| —C₁₁H₂₃ | —C₁₃H₂₇ |
| —C₁₁H₂₃ | —C₁₅H₃₁ |
| —C₁₁H₂₃ | —C₁₇H₃₅ |
| —C₁₁H₂₃ | 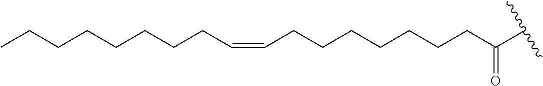 [SMILES: *C(=O)CCCCCCC/C=C\CCCCCCCC] |
| —C₁₁H₂₃ | 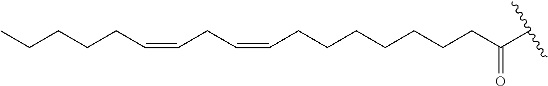 [SMILES: *C(=O)CCCCCCC/C=C\C/C=C\CCCCC] |
| —C₁₁H₂₃ | 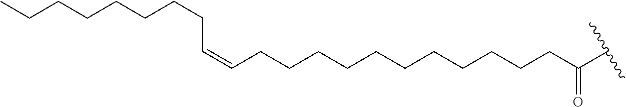 [SMILES: *C(=O)CCCCCCCCCCC/C=C\CCCCCCCC] |
| —C₁₁H₂₃ | 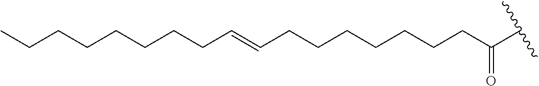 [SMILES: *C(=O)CCCCCCC/C=C/CCCCCCCC] |
| —C₁₁H₂₃ | 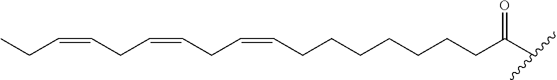 [SMILES: *C(=O)CCCCCCC/C=C\C/C=C\C/C=C\CC] |
| —C₁₁H₂₃ | tBu |
| —C₁₁H₂₃ | phenyl |
| —C₁₁H₂₃ | 4-chlorophenyl |
| —C₁₁H₂₃ | 3,4-dichlorophenyl |
| —C₁₁H₂₃ | 4-methylphenyl |
| —C₁₁H₂₃ | 4-methoxyphenyl |
| —C₁₃H₂₇ | Me |
| —C₁₃H₂₇ | —C₇H₁₅ |
| —C₁₃H₂₇ | —C₉H₁₉ |
| —C₁₃H₂₇ | —C₁₁H₂₃ |
| —C₁₃H₂₇ | —C₁₃H₂₇ |
| —C₁₃H₂₇ | —C₁₅H₃₁ |
| —C₁₃H₂₇ | —C₁₇H₃₅ |
| —C₁₃H₂₇ | 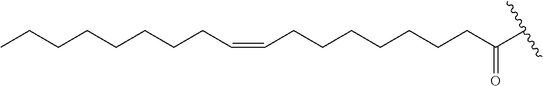 [SMILES: *C(=O)CCCCCCC/C=C\CCCCCCCC] |
| —C₁₃H₂₇ | 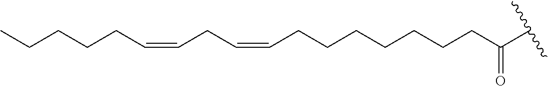 [SMILES: *C(=O)CCCCCCC/C=C\C/C=C\CCCCC] |
| —C₁₃H₂₇ | 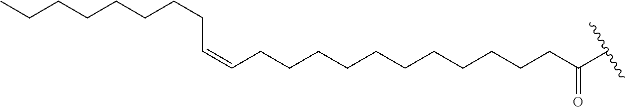 [SMILES: *C(=O)CCCCCCCCCCC/C=C\CCCCCCCC] |
| —C₁₃H₂₇ | 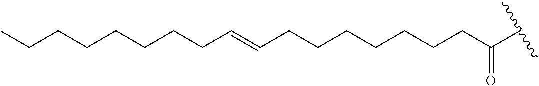 [SMILES: *C(=O)CCCCCCC/C=C/CCCCCCCC] |
| —C₁₃H₂₇ | 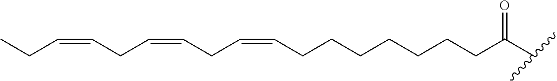 [SMILES: *C(=O)CCCCCCC/C=C\C/C=C\C/C=C\CC] |
| —C₁₃H₂₇ | tBu |
| —C₁₃H₂₇ | phenyl |
| —C₁₃H₂₇ | 4-chlorophenyl |
| —C₁₃H₂₇ | 3,4-dichlorophenyl |
| —C₁₃H₂₇ | 4-methylphenyl |
| —C₁₃H₂₇ | 4-methoxyphenyl |
| —C₁₅H₃₁ | Me |
| —C₁₅H₃₁ | —C₇H₁₅ |
| —C₁₅H₃₁ | —C₉H₁₉ |
| —C₁₅H₃₁ | —C₁₁H₂₃ |
| —C₁₅H₃₁ | —C₁₃H₂₇ |
| —C₁₅H₃₁ | —C₁₅H₃₁ |
| —C₁₅H₃₁ | —C₁₇H₃₅ |
| —C₁₅H₃₁ | 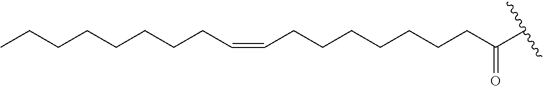 [SMILES: *C(=O)CCCCCCC/C=C\CCCCCCCC] |
| —C₁₅H₃₁ | 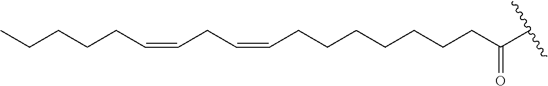 [SMILES: *C(=O)CCCCCCC/C=C\C/C=C\CCCCC] |
| —C₁₅H₃₁ | 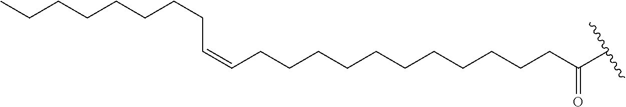 [SMILES: *C(=O)CCCCCCCCCCC/C=C\CCCCCCCC] |
| —C₁₅H₃₁ | 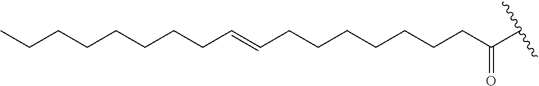 [SMILES: *C(=O)CCCCCCC/C=C/CCCCCCCC] |
| —C₁₅H₃₁ | 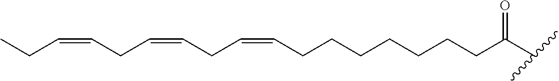 [SMILES: *C(=O)CCCCCCC/C=C\C/C=C\C/C=C\CC] |
| —C₁₅H₃₁ | tBu |
| —C₁₅H₃₁ | phenyl |
| —C₁₅H₃₁ | 4-chlorophenyl |
| —C₁₅H₃₁ | 3,4-dichlorophenyl |
| —C₁₅H₃₁ | 4-methylphenyl |
| —C₁₅H₃₁ | 4-methoxyphenyl |
| —C₁₇H₃₅ | Me |
| —C₁₇H₃₅ | —C₇H₁₅ |
| —C₁₇H₃₅ | —C₉H₁₉ |
| —C₁₇H₃₅ | —C₁₁H₂₃ |
| —C₁₇H₃₅ | —C₁₃H₂₇ |
| —C₁₇H₃₅ | —C₁₅H₃₁ |
| —C₁₇H₃₅ | —C₁₇H₃₅ |
| —C₁₇H₃₅ | 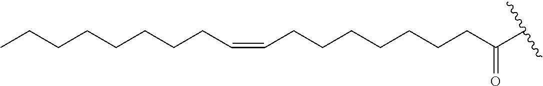 [SMILES: *C(=O)CCCCCCC/C=C\CCCCCCCC] |
| —C₁₇H₃₅ | 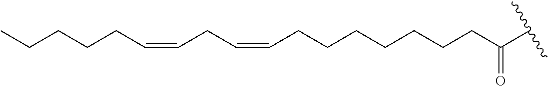 [SMILES: *C(=O)CCCCCCC/C=C\C/C=C\CCCCC] |
| —C₁₇H₃₅ | 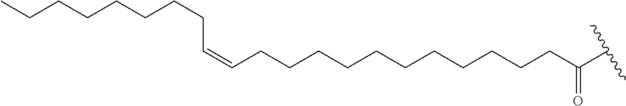 [SMILES: *C(=O)CCCCCCCCCCC/C=C\CCCCCCCC] |
| —C₁₇H₃₅ | 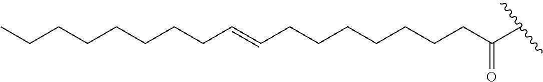 [SMILES: *C(=O)CCCCCCC/C=C/CCCCCCCC] |
| —C₁₇H₃₅ | 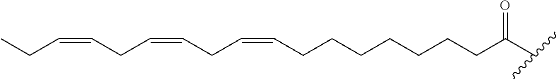 [SMILES: *C(=O)CCCCCCC/C=C\C/C=C\C/C=C\CC] |
| —C₁₇H₃₅ | tBu |
| —C₁₇H₃₅ | phenyl |
| —C₁₇H₃₅ | 4-chlorophenyl |
| —C₁₇H₃₅ | 3,4-dichlorophenyl |
| —C₁₇H₃₅ | 4-methylphenyl |
| —C₁₇H₃₅ | 4-methoxyphenyl |
| Phenyl | Me |
| Phenyl | —C₇H₁₅ |
| Phenyl | —C₉H₁₉ |
| Phenyl | —C₁₁H₂₃ |
| Phenyl | —C₁₃H₂₇ |
| Phenyl | —C₁₅H₃₁ |
| Phenyl | —C₁₇H₃₅ |
| Phenyl | 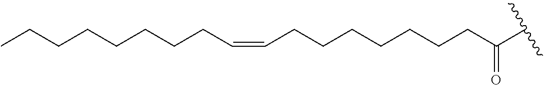 [SMILES: *C(=O)CCCCCCC/C=C\CCCCCCCC] |
| Phenyl | 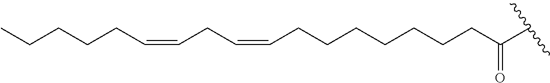 [SMILES: *C(=O)CCCCCCC/C=C\C/C=C\CCCCC] |
| Phenyl | 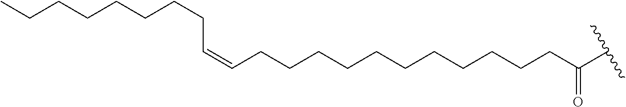 [SMILES: *C(=O)CCCCCCCCCCC/C=C\CCCCCCCC] |
| Phenyl | 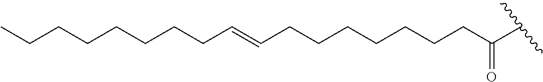 [SMILES: *C(=O)CCCCCCC/C=C/CCCCCCCC] |
| Phenyl | 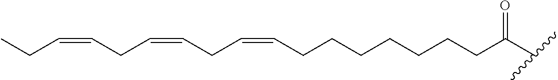 [SMILES: *C(=O)CCCCCCC/C=C\C/C=C\C/C=C\CC] |
| Phenyl | tBu |
| Phenyl | phenyl |
| Phenyl | 4-chlorophenyl |
| Phenyl | 3,4-dichlorophenyl |
| Phenyl | 4-methylphenyl |
| Phenyl | 4-methoxyphenyl |
| 4-chlorophenyl | Me |
| 4-chlorophenyl | —C₇H₁₅ |
| 4-chlorophenyl | —C₉H₁₉ |
| 4-chlorophenyl | —C₁₁H₂₃ |
| 4-chlorophenyl | —C₁₃H₂₇ |
| 4-chlorophenyl | —C₁₅H₃₁ |
| 4-chlorophenyl | —C₁₇H₃₅ |
| 4-chlorophenyl | 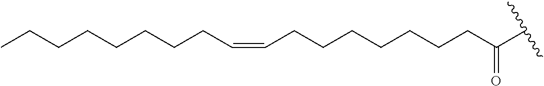 [SMILES: *C(=O)CCCCCCC/C=C\CCCCCCCC] |
| 4-chlorophenyl | 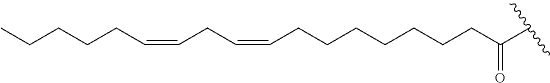 [SMILES: *C(=O)CCCCCCC/C=C\C/C=C\CCCCC] |
| 4-chlorophenyl | 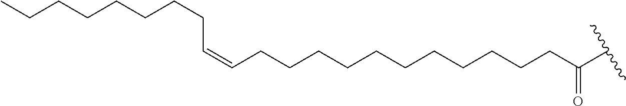 [SMILES: *C(=O)CCCCCCCCCCC/C=C\CCCCCCCC] |
| 4-chlorophenyl | 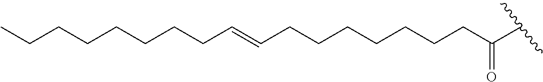 [SMILES: *C(=O)CCCCCCC/C=C/CCCCCCCC] |
| 4-chlorophenyl | 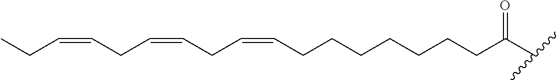 [SMILES: *C(=O)CCCCCCC/C=C\C/C=C\C/C=C\CC] |
| 4-chlorophenyl | tBu |
| 4-chlorophenyl | phenyl |
| 4-chlorophenyl | 4-chlorophenyl |
| 4-chlorophenyl | 3,4-dichlorophenyl |
| 4-chlorophenyl | 4-methylphenyl |
| 4-chlorophenyl | 4-methoxyphenyl |
| 3,4-dichlorophenyl | Me |
| 3,4-dichlorophenyl | —C₇H₁₅ |
| 3,4-dichlorophenyl | —C₉H₁₉ |
| 3,4-dichlorophenyl | —C₁₁H₂₃ |
| 3,4-dichlorophenyl | —C₁₃H₂₇ |
| 3,4-dichlorophenyl | —C₁₅H₃₁ |
| 3,4-dichlorophenyl | —C₁₇H₃₅ |
| 3,4-dichlorophenyl | 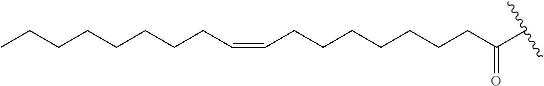 [SMILES: *C(=O)CCCCCCC/C=C\CCCCCCCC] |
| 3,4-dichlorophenyl | 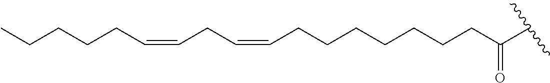 [SMILES: *C(=O)CCCCCCC/C=C\C/C=C\CCCCC] |
| 3,4-dichlorophenyl | 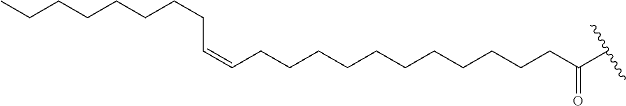 [SMILES: *C(=O)CCCCCCCCCCC/C=C\CCCCCCCC] |
| 3,4-dichlorophenyl | 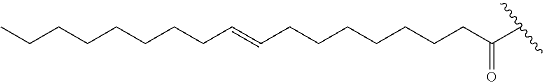 [SMILES: *C(=O)CCCCCCC/C=C/CCCCCCCC] |
| 3,4-dichlorophenyl | 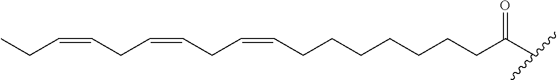 [SMILES: *C(=O)CCCCCCC/C=C\C/C=C\C/C=C\CC] |
| 3,4-dichlorophenyl | tBu |
| 3,4-dichlorophenyl | phenyl |
| 3,4-dichlorophenyl | 4-chlorophenyl |
| 3,4-dichlorophenyl | 3,4-dichlorophenyl |
| 3,4-dichlorophenyl | 4-methylphenyl |
| 3,4-dichlorophenyl | 4-methoxyphenyl |
| 4-methylphenyl | Me |
| 4-methylphenyl | —C₇H₁₅ |
| 4-methylphenyl | —C₉H₁₉ |
| 4-methylphenyl | —C₁₁H₂₃ |
| 4-methylphenyl | —C₁₃H₂₇ |
| 4-methylphenyl | —C₁₅H₃₁ |
| 4-methylphenyl | —C₁₇H₃₅ |
| 4-methylphenyl | 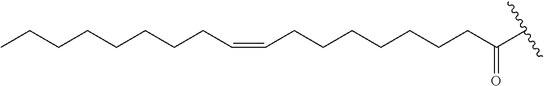 [SMILES: *C(=O)CCCCCCC/C=C\CCCCCCCC] |
| 4-methylphenyl | 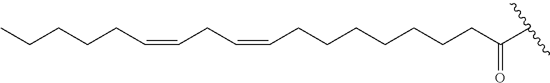 [SMILES: *C(=O)CCCCCCC/C=C\C/C=C\CCCCC] |
| 4-methylphenyl | 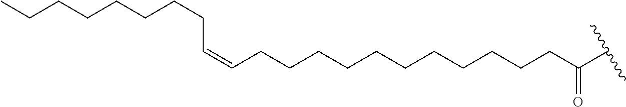 [SMILES: *C(=O)CCCCCCCCCCC/C=C\CCCCCCCC] |
| 4-methylphenyl | 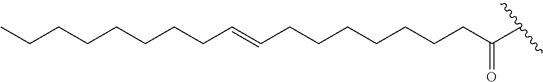 [SMILES: *C(=O)CCCCCCC/C=C/CCCCCCCC] |
| 4-methylphenyl | 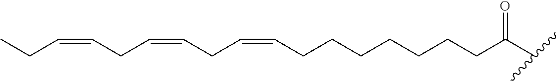 [SMILES: *C(=O)CCCCCCC/C=C\C/C=C\C/C=C\CC] |
| 4-methylphenyl | tBu |
| 4-methylphenyl | phenyl |
| 4-methylphenyl | 4-chlorophenyl |
| 4-methylphenyl | 3,4-dichlorophenyl |
| 4-methylphenyl | 4-methylphenyl |
| 4-methylphenyl | 4-methoxyphenyl |
| 4-methoxyphenyl | Me |
| 4-methoxyphenyl | —C₇H₁₅ |
| 4-methoxyphenyl | —C₉H₁₉ |
| 4-methoxyphenyl | —C₁₁H₂₃ |
| 4-methoxyphenyl | —C₁₃H₂₇ |
| 4-methoxyphenyl | —C₁₅H₃₁ |
| 4-methoxyphenyl | —C₁₇H₃₅ |
| 4-methoxyphenyl | 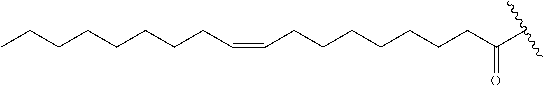 [SMILES: *C(=O)CCCCCCC/C=C\CCCCCCCC] |
| 4-methoxyphenyl | 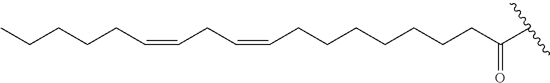 [SMILES: *C(=O)CCCCCCC/C=C\C/C=C\CCCCC] |
| 4-methoxyphenyl | 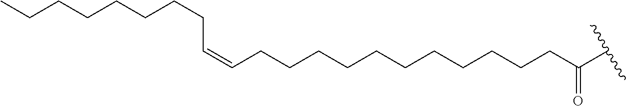 [SMILES: *C(=O)CCCCCCCCCCC/C=C\CCCCCCCC] |
| 4-methoxyphenyl | 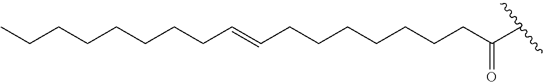 [SMILES: *C(=O)CCCCCCC/C=C/CCCCCCCC] |
| 4-methoxyphenyl | 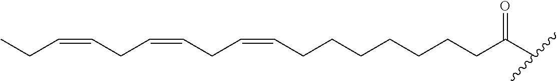 [SMILES: *C(=O)CCCCCCC/C=C\C/C=C\C/C=C\CC] |
| 4-methoxyphenyl | tBu |
| 4-methoxyphenyl | phenyl |
| 4-methoxyphenyl | 4-chlorophenyl |
| 4-methoxyphenyl | 3,4-dichlorophenyl |
| 4-methoxyphenyl | 4-methylphenyl |
| 4-methoxyphenyl | 4-methoxyphenyl |
| 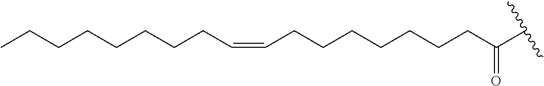 [SMILES: *C(=O)CCCCCCC/C=C\CCCCCCCC] | Me |
| 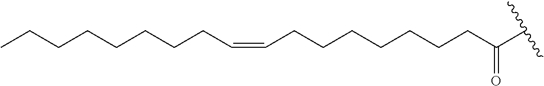 [SMILES: *C(=O)CCCCCCC/C=C\CCCCCCCC] | —C₇H₁₅ |
| 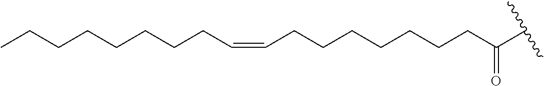 [SMILES: *C(=O)CCCCCCC/C=C\CCCCCCCC] | —C₉ H₁₉ |
| 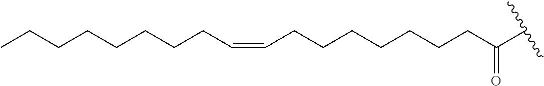 [SMILES: *C(=O)CCCCCCC/C=C\CCCCCCCC] | —C₁₁H₂₃ |
| 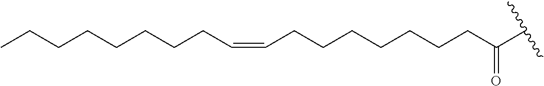 [SMILES: *C(=O)CCCCCCC/C=C\CCCCCCCC] | —C₁₃H₂₇ |
| 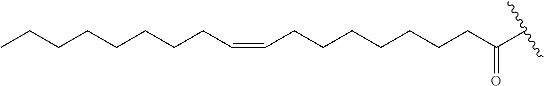 [SMILES: *C(=O)CCCCCCC/C=C\CCCCCCCC] | —C₁₅H₃₁ |
| 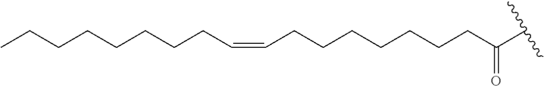 [SMILES: *C(=O)CCCCCCC/C=C\CCCCCCCC] | —C₁₇H₃₅ |
| 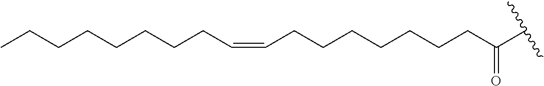 [SMILES: *C(=O)CCCCCCC/C=C\CCCCCCCC] | 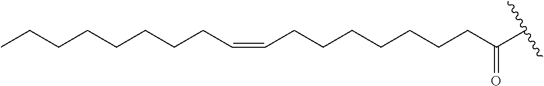 [SMILES: *C(=O)CCCCCCC/C=C\CCCCCCCC] |
| 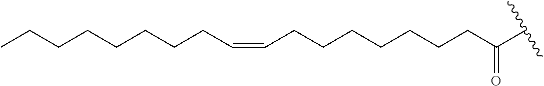 [SMILES: *C(=O)CCCCCCC/C=C\CCCCCCCC] | 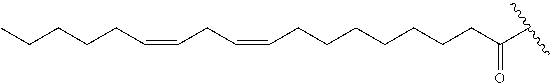 [SMILES: *C(=O)CCCCCCC/C=C\C/C=C\CCCCC] |
| 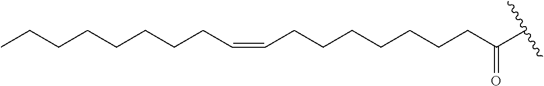 [SMILES: *C(=O)CCCCCCC/C=C\CCCCCCCC] | 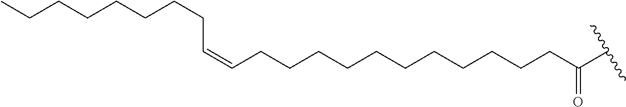 [SMILES: *C(=O)CCCCCCCCCCC/C=C\CCCCCCCC] |
| 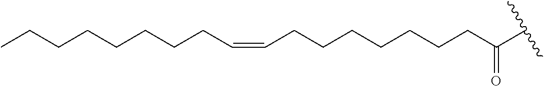 [SMILES: *C(=O)CCCCCCC/C=C\CCCCCCCC] | 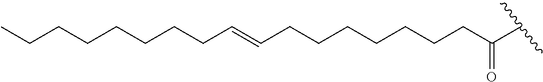 [SMILES: *C(=O)CCCCCCC/C=C/CCCCCCCC] |
| 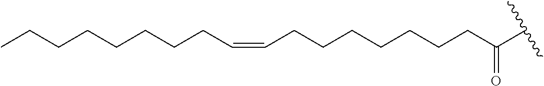 [SMILES: *C(=O)CCCCCCC/C=C\CCCCCCCC] | 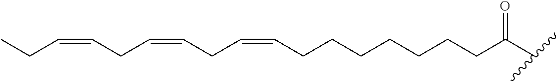 [SMILES: *C(=O)CCCCCCC/C=C\C/C=C\C/C=C\CC] |
| 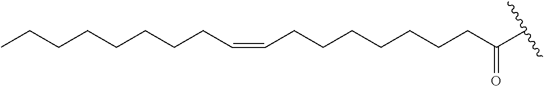 [SMILES: *C(=O)CCCCCCC/C=C\CCCCCCCC] | tBu |
| 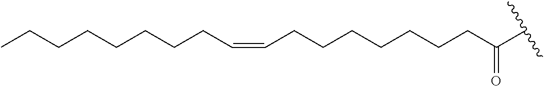 [SMILES: *C(=O)CCCCCCC/C=C\CCCCCCCC] | phenyl |
| 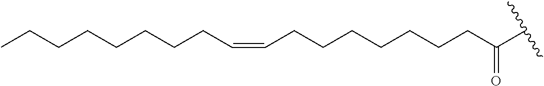 [SMILES: *C(=O)CCCCCCC/C=C\CCCCCCCC] | 4-chlorophenyl |
| 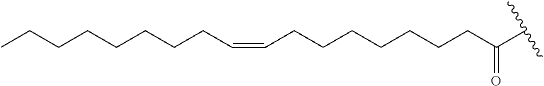 [SMILES: *C(=O)CCCCCCC/C=C\CCCCCCCC] | 3,4-dichlorophenyl |
| 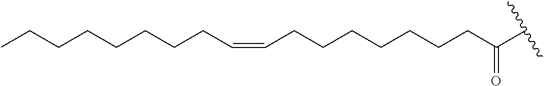 [SMILES: *C(=O)CCCCCCC/C=C\CCCCCCCC] | 4-methylphenyl |
| 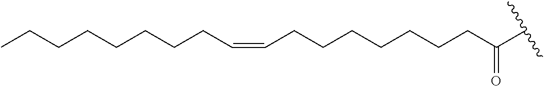 [SMILES: *C(=O)CCCCCCC/C=C\CCCCCCCC] | 4-methoxyphenyl |
| 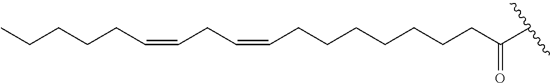 [SMILES: *C(=O)CCCCCCC/C=C\C/C=C\CCCCC] | Me |
| 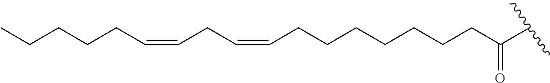 [SMILES: *C(=O)CCCCCCC/C=C\C/C=C\CCCCC] | —C₇H₁₅ |
| 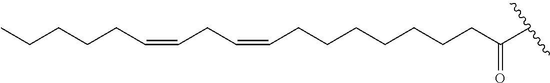 [SMILES: *C(=O)CCCCCCC/C=C\C/C=C\CCCCC] | —C₉H₁₉ |
| 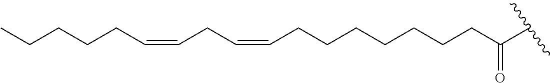 [SMILES: *C(=O)CCCCCCC/C=C\C/C=C\CCCCC] | —C₁₁H₂₃ |
| 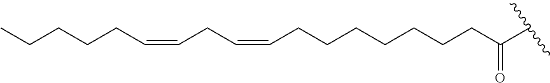 [SMILES: *C(=O)CCCCCCC/C=C\C/C=C\CCCCC] | —C₁₃H₂₇ |
| 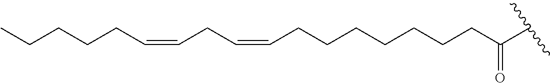 [SMILES: *C(=O)CCCCCCC/C=C\C/C=C\CCCCC] | —C₁₅H₃₁ |
| 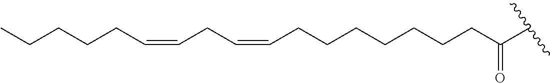 [SMILES: *C(=O)CCCCCCC/C=C\C/C=C\CCCCC] | —C₁₇H₃₅ |
| 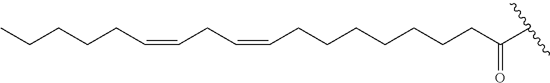 [SMILES: *C(=O)CCCCCCC/C=C\C/C=C\CCCCC] | 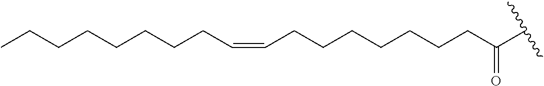 [SMILES: *C(=O)CCCCCCC/C=C\CCCCCCCC] |
| 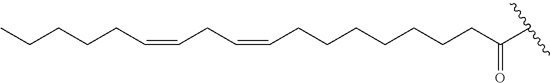 [SMILES: *C(=O)CCCCCCC/C=C\C/C=C\CCCCC] | 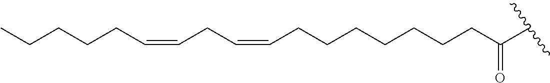 [SMILES: *C(=O)CCCCCCC/C=C\C/C=C\CCCCC] |
| 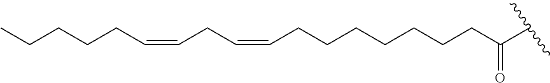 [SMILES: *C(=O)CCCCCCC/C=C\C/C=C\CCCCC] | 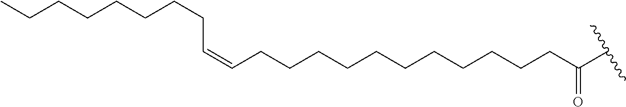 [SMILES: *C(=O)CCCCCCCCCCC/C=C\CCCCCCCC] |
| 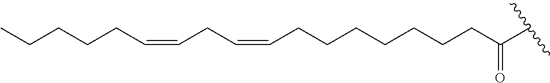 [SMILES: *C(=O)CCCCCCC/C=C\C/C=C\CCCCC] | 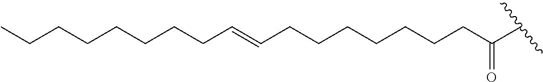 [SMILES: *C(=O)CCCCCCC/C=C/CCCCCCCC] |
| 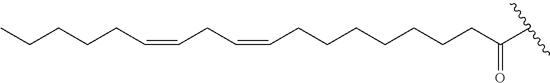 [SMILES: *C(=O)CCCCCCC/C=C\C/C=C\CCCCC] | 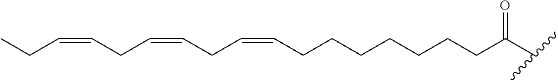 [SMILES: *C(=O)CCCCCCC/C=C\C/C=C\C/C=C\CC] |
| 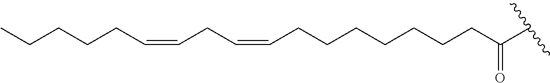 [SMILES: *C(=O)CCCCCCC/C=C\C/C=C\CCCCC] | tBu |
| 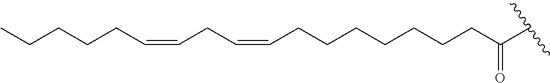 [SMILES: *C(=O)CCCCCCC/C=C\C/C=C\CCCCC] | phenyl |
| 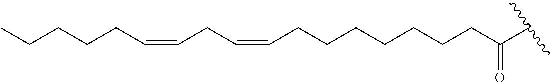 [SMILES: *C(=O)CCCCCCC/C=C\C/C=C\CCCCC] | 4-chlorophenyl |
| 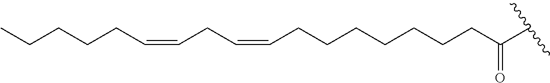 [SMILES: *C(=O)CCCCCCC/C=C\C/C=C\CCCCC] | 3,4-dichlorophenyl |
| 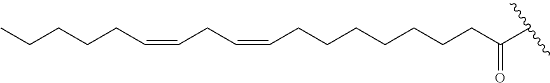 [SMILES: *C(=O)CCCCCCC/C=C\C/C=C\CCCCC] | 4-methylphenyl |
| 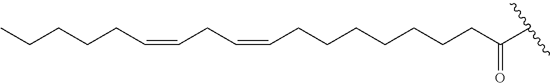 [SMILES: *C(=O)CCCCCCC/C=C\C/C=C\CCCCC] | 4-methoxyphenyl |
| 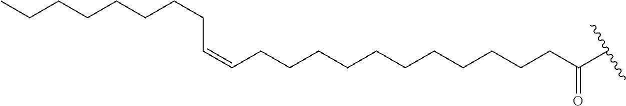 [SMILES: *C(=O)CCCCCCCCCCC/C=C\CCCCCCCC] | Me |
| 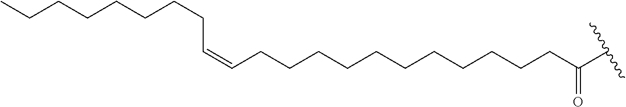 [SMILES: *C(=O)CCCCCCCCCCC/C=C\CCCCCCCC] | —C₇H₁₅ |
| 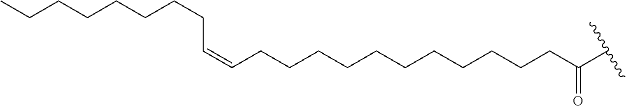 [SMILES: *C(=O)CCCCCCCCCCC/C=C\CCCCCCCC] | —C₉H₁₉ |
| 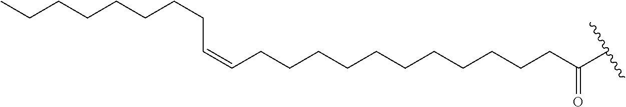 [SMILES: *C(=O)CCCCCCCCCCC/C=C\CCCCCCCC] | —C₁₁H₂₃ |
| 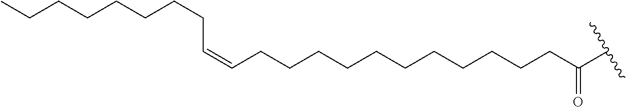 [SMILES: *C(=O)CCCCCCCCCCC/C=C\CCCCCCCC] | —C₁₃H₂₇ |
| 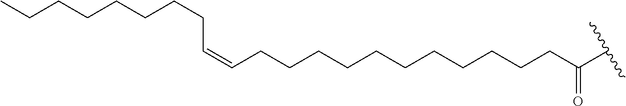 [SMILES: *C(=O)CCCCCCCCCCC/C=C\CCCCCCCC] | —C₁₅H₃₁ |
| 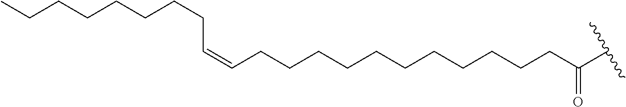 [SMILES: *C(=O)CCCCCCCCCCC/C=C\CCCCCCCC] | —C₁₇H₃₅ |
| 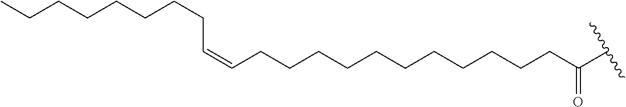 [SMILES: *C(=O)CCCCCCCCCCC/C=C\CCCCCCCC] | 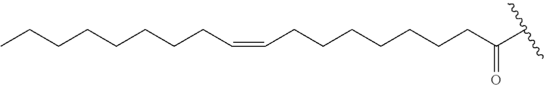 [SMILES: *C(=O)CCCCCCC/C=C\CCCCCCCC] |
| 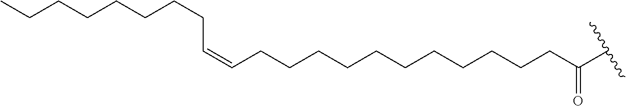 [SMILES: *C(=O)CCCCCCCCCCC/C=C\CCCCCCCC] | 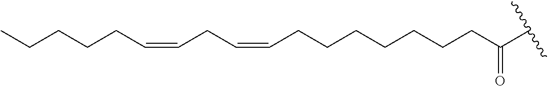 [SMILES: *C(=O)CCCCCCC/C=C\C/C=C\CCCCC] |
| 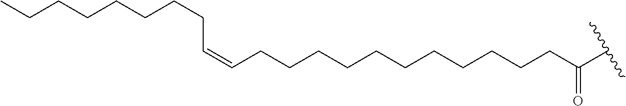 [SMILES: *C(=O)CCCCCCCCCCC/C=C\CCCCCCCC] | 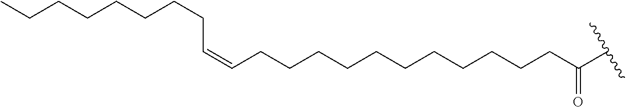 [SMILES: *C(=O)CCCCCCCCCCC/C=C\CCCCCCCC] |
| 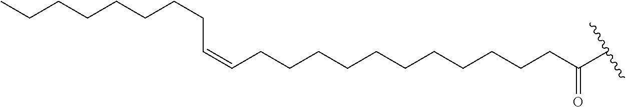 [SMILES: *C(=O)CCCCCCCCCCC/C=C\CCCCCCCC] | 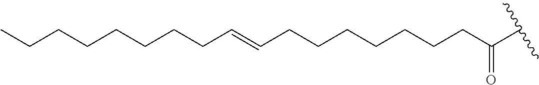 [SMILES: *C(=O)CCCCCCC/C=C/CCCCCCCC] |
| 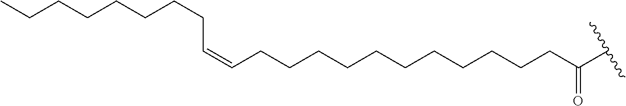 [SMILES: *C(=O)CCCCCCCCCCC/C=C\CCCCCCCC] | 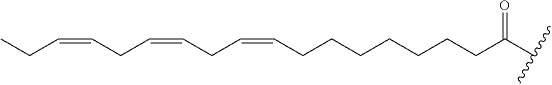 [SMILES: *C(=O)CCCCCCC/C=C\C/C=C\C/C=C\CC] |
| 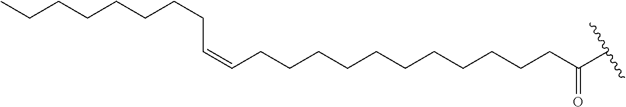 [SMILES: *C(=O)CCCCCCCCCCC/C=C\CCCCCCCC] | tBu |
| 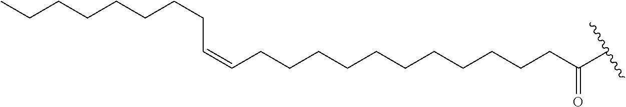 [SMILES: *C(=O)CCCCCCCCCCC/C=C\CCCCCCCC] | phenyl |
| 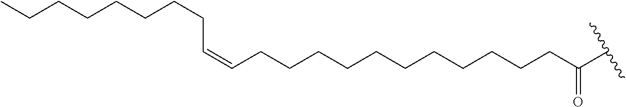 [SMILES: *C(=O)CCCCCCCCCCC/C=C\CCCCCCCC] | 4-chlorophenyl |
| 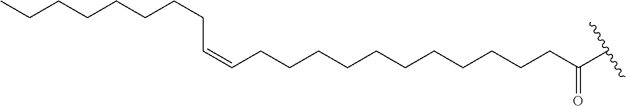 [SMILES: *C(=O)CCCCCCCCCCC/C=C\CCCCCCCC] | 3,4-dichlorophenyl |
| 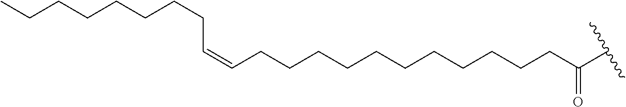 [SMILES: *C(=O)CCCCCCCCCCC/C=C\CCCCCCCC] | 4-methylphenyl |
| 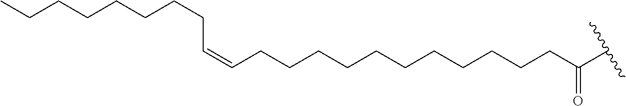 [SMILES: *C(=O)CCCCCCCCCCC/C=C\CCCCCCCC] | 4-methoxyphenyl |
| 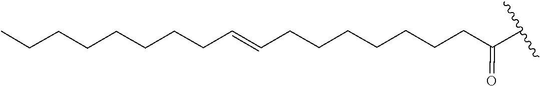 [SMILES: *C(=O)CCCCCCC/C=C/CCCCCCCC] | Me |
| 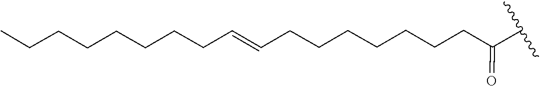 [SMILES: *C(=O)CCCCCCC/C=C/CCCCCCCC] | —C₇H₁₅ |
| 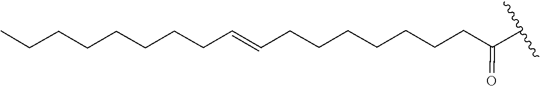 [SMILES: *C(=O)CCCCCCC/C=C/CCCCCCCC] | —C₉H₁₉ |
| 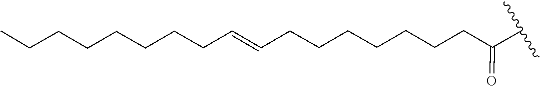 [SMILES: *C(=O)CCCCCCC/C=C/CCCCCCCC] | —C₁₁H₂₃ |
| 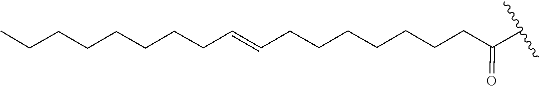 [SMILES: *C(=O)CCCCCCC/C=C/CCCCCCCC] | —C₁₃H₂₇ |
| 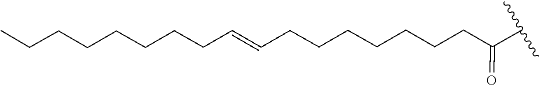 [SMILES: *C(=O)CCCCCCC/C=C/CCCCCCCC] | —C₁₅H₃₁ |
| 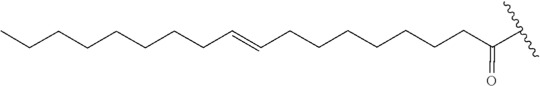 [SMILES: *C(=O)CCCCCCC/C=C/CCCCCCCC] | —C₁₇H₃₅ |
| 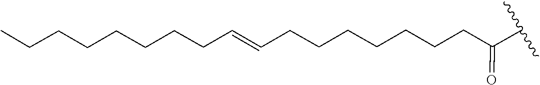 [SMILES: *C(=O)CCCCCCC/C=C/CCCCCCCC] | 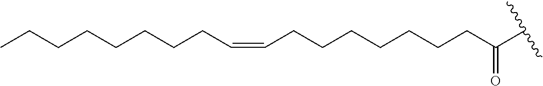 [SMILES: *C(=O)CCCCCCC/C=C\CCCCCCCC] |
| 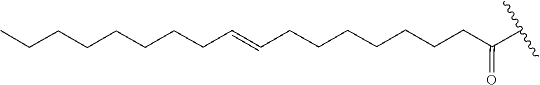 [SMILES: *C(=O)CCCCCCC/C=C/CCCCCCCC] | 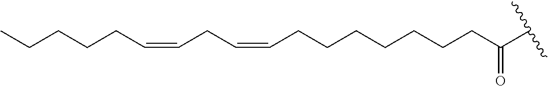 [SMILES: *C(=O)CCCCCCC/C=C\C/C=C\CCCCC] |
| 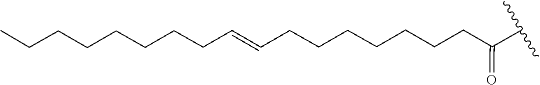 [SMILES: *C(=O)CCCCCCC/C=C/CCCCCCCC] | 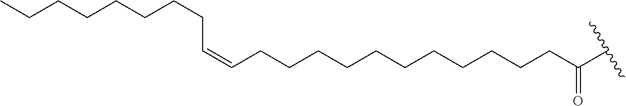 [SMILES: *C(=O)CCCCCCCCCCC/C=C\CCCCCCCC] |
| 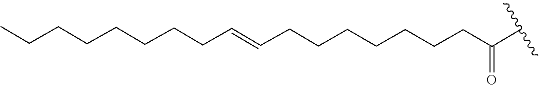 [SMILES: *C(=O)CCCCCCC/C=C/CCCCCCCC] | 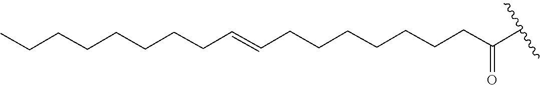 [SMILES: *C(=O)CCCCCCC/C=C/CCCCCCCC] |
| 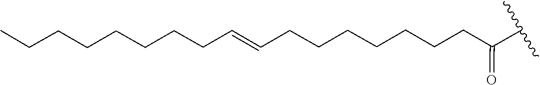 [SMILES: *C(=O)CCCCCCC/C=C/CCCCCCCC] | 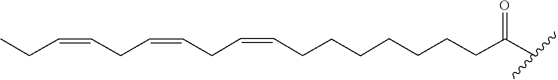 [SMILES: *C(=O)CCCCCCC/C=C\C/C=C\C/C=C\CC] |
| 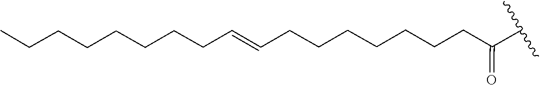 [SMILES: *C(=O)CCCCCCC/C=C/CCCCCCCC] | tBu |
| 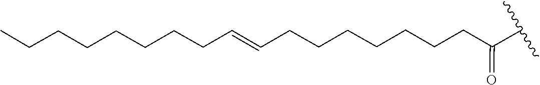 [SMILES: *C(=O)CCCCCCC/C=C/CCCCCCCC] | phenyl |
| 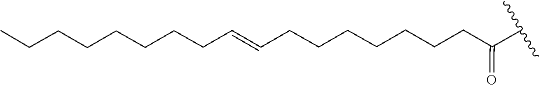 [SMILES: *C(=O)CCCCCCC/C=C/CCCCCCCC] | 4-chlorophenyl |
| 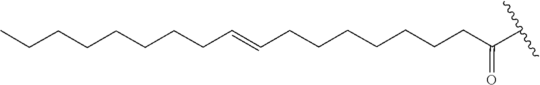 [SMILES: *C(=O)CCCCCCC/C=C/CCCCCCCC] | 3,4-dichlorophenyl |
| 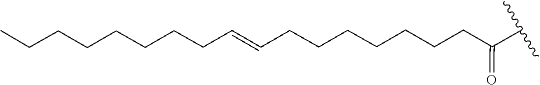 [SMILES: *C(=O)CCCCCCC/C=C/CCCCCCCC] | 4-methylphenyl |
| 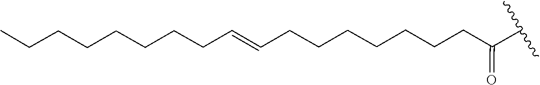 [SMILES: *C(=O)CCCCCCC/C=C/CCCCCCCC] | 4-methoxyphenyl |
| 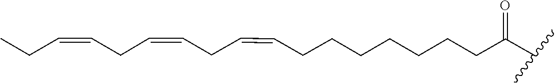 [SMILES: *C(=O)CCCCCCC/C=C\C/C=C\C/C=C\CC] | Me |
| 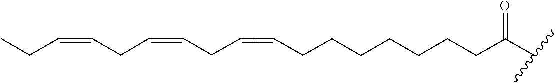 [SMILES: *C(=O)CCCCCCC/C=C\C/C=C\C/C=C\CC] | —C₇H₁₅ |
| 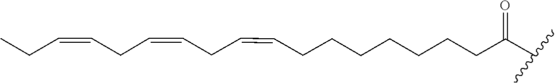 [SMILES: *C(=O)CCCCCCC/C=C\C/C=C\C/C=C\CC] | —C₉H₁₉ |
| 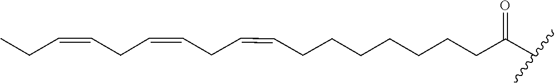 [SMILES: *C(=O)CCCCCCC/C=C\C/C=C\C/C=C\CC] | —C₁₁H₂₃ |
| 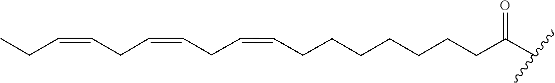 [SMILES: *C(=O)CCCCCCC/C=C\C/C=C\C/C=C\CC] | —C₁₃H₂₇ |
| 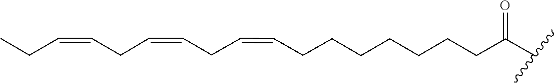 [SMILES: *C(=O)CCCCCCC/C=C\C/C=C\C/C=C\CC] | —C₁₅H₃₁ |
| 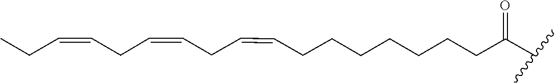 [SMILES: *C(=O)CCCCCCC/C=C\C/C=C\C/C=C\CC] | —C₁₇H₃₅ |
| 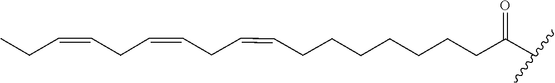 [SMILES: *C(=O)CCCCCCC/C=C\C/C=C\C/C=C\CC] | 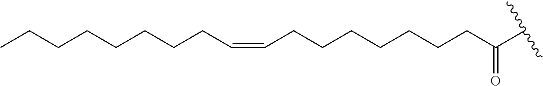 [SMILES: *C(=O)CCCCCCC/C=C\CCCCCCCC] |
| 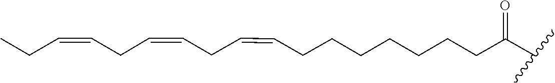 [SMILES: *C(=O)CCCCCCC/C=C\C/C=C\C/C=C\CC] | 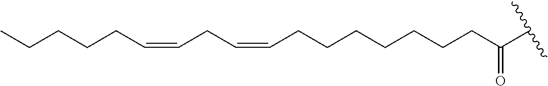 [SMILES: *C(=O)CCCCCCC/C=C\C/C=C\CCCCC] |
| 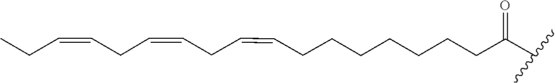 [SMILES: *C(=O)CCCCCCC/C=C\C/C=C\C/C=C\CC] | 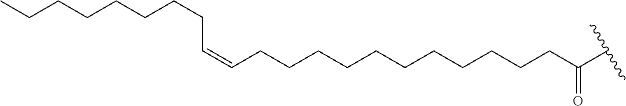 [SMILES: *C(=O)CCCCCCCCCCC/C=C\CCCCCCCC] |
| 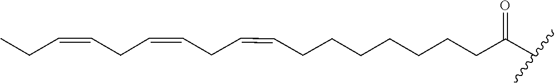 [SMILES: *C(=O)CCCCCCC/C=C\C/C=C\C/C=C\CC] | 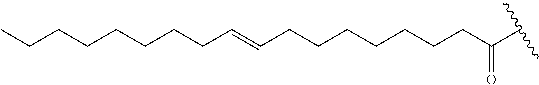 [SMILES: *C(=O)CCCCCCC/C=C/CCCCCCCC] |
| 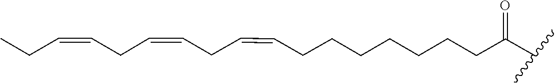 [SMILES: *C(=O)CCCCCCC/C=C\C/C=C\C/C=C\CC] | 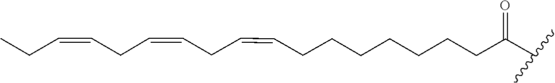 [SMILES: *C(=O)CCCCCCC/C=C\C/C=C\C/C=C\CC] |
| 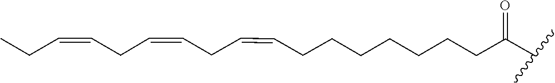 [SMILES: *C(=O)CCCCCCC/C=C\C/C=C\C/C=C\CC] | tBu |
| 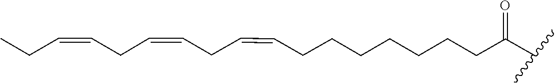 [SMILES: *C(=O)CCCCCCC/C=C\C/C=C\C/C=C\CC] | phenyl |
| 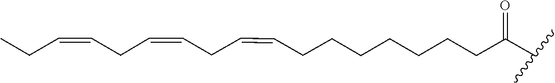 [SMILES: *C(=O)CCCCCCC/C=C\C/C=C\C/C=C\CC] | 4-chlorophenyl |
| 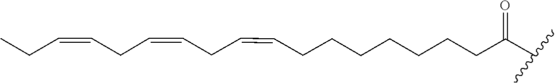 [SMILES: *C(=O)CCCCCCC/C=C\C/C=C\C/C=C\CC] | 3,4-dichlorophenyl |
| 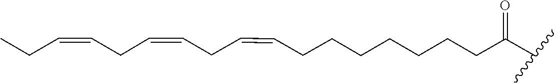 [SMILES: *C(=O)CCCCCCC/C=C\C/C=C\C/C=C\CC] | 4-methylphenyl |
| 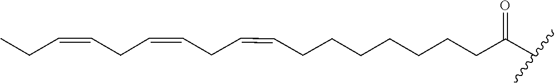 [SMILES: *C(=O)CCCCCCC/C=C\C/C=C\C/C=C\CC] | 4-methoxyphenyl |
| H | 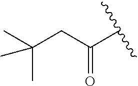 [SMILES: *C(=O)CC(C)(C)C] |
| H | 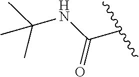 [SMILES: *C(=O)NC(C)(C)C] |
| H | 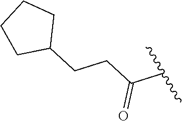 [SMILES: *C(=O)CCC1CCCC1] |
| H | 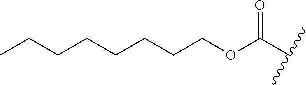 [SMILES: *C(=O)OCCCCCCCC] |
| H | 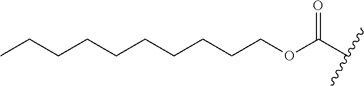 [SMILES: *C(=O)OCCCCCCCCCC] |
| H | 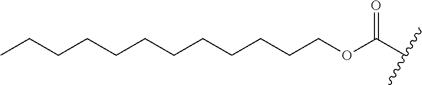 [SMILES: *C(=O)OCCCCCCCCCCCC] |
| H | 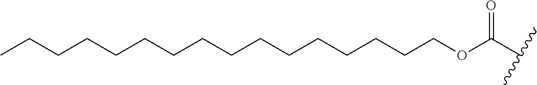 [SMILES: *C(=O)OCCCCCCCCCCCCCCCC] |
| H | 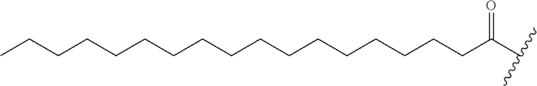 [SMILES: *C(=O)CCCCCCCCCCCCCCCCC] |
| H | 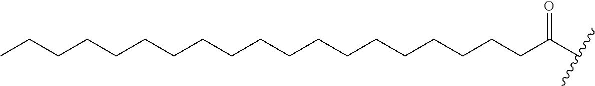 [SMILES: *C(=O)CCCCCCCCCCCCCCCCCCC] |
| H | 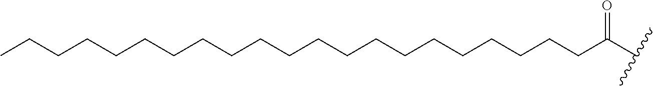 [SMILES: *C(=O)CCCCCCCCCCCCCCCCCCCCC] |
| H | 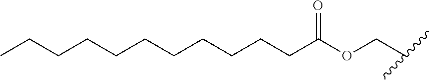 [SMILES: *COC(=O)CCCCCCCCCCC] |
| H | 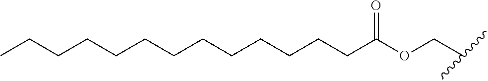 [SMILES: *COC(=O)CCCCCCCCCCCCC] |
| H | 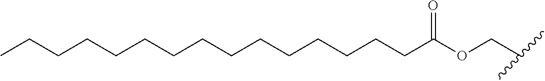 [SMILES: *COC(=O)CCCCCCCCCCCCCCC] |
| H | 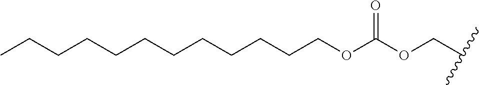 [SMILES: *COC(=O)OCCCCCCCCCCCC] |
| H | 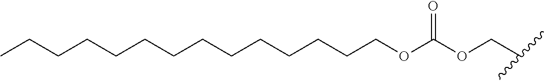 [SMILES: *COC(=O)OCCCCCCCCCCCCCC] |
| H | 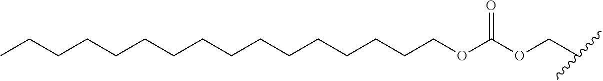 [SMILES: *COC(=O)OCCCCCCCCCCCCCCCC] |
| Me | 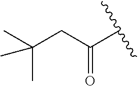 [SMILES: *C(=O)CC(C)(C)C] |
| Me | 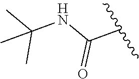 [SMILES: *C(=O)NC(C)(C)C] |
| Me | 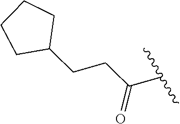 [SMILES: *C(=O)CCC1CCCC1] |
| Me | 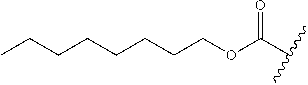 [SMILES: *C(=O)OCCCCCCCC] |
| Me | 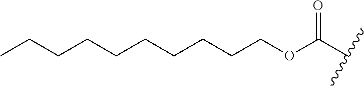 [SMILES: *C(=O)OCCCCCCCCCC] |
| Me | 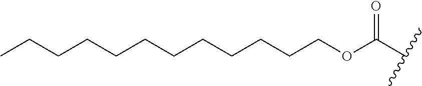 [SMILES: *C(=O)OCCCCCCCCCCCC] |
| Me | 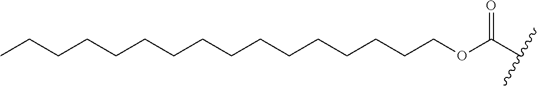 [SMILES: *C(=O)OCCCCCCCCCCCCCCCC] |
| Me | 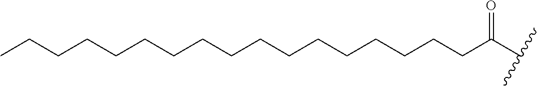 [SMILES: *C(=O)CCCCCCCCCCCCCCCCC] |
| Me | 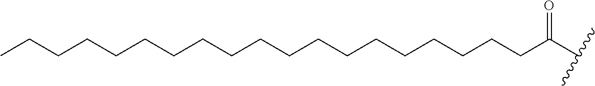 [SMILES: *C(=O)CCCCCCCCCCCCCCCCCCC] |
| Me | 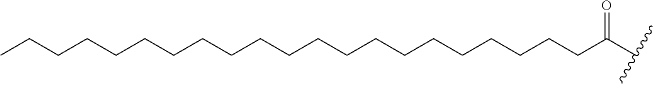 [SMILES: *C(=O)CCCCCCCCCCCCCCCCCCCCC] |
| Me | 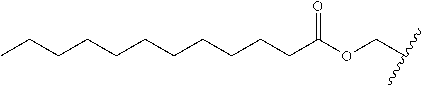 [SMILES: *COC(=O)CCCCCCCCCCC] |
| Me | 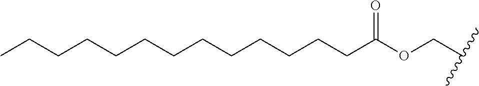 [SMILES: *COC(=O)CCCCCCCCCCCCC] |
| Me | 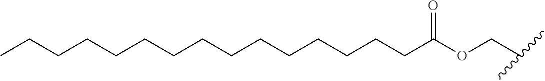 [SMILES: *COC(=O)CCCCCCCCCCCCCCC] |
| Me | 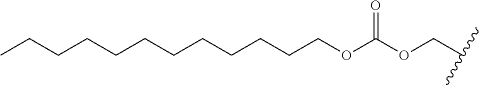 [SMILES: *COC(=O)OCCCCCCCCCCCC] |
| Me | 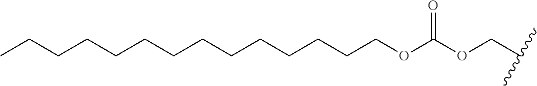 [SMILES: *COC(=O)OCCCCCCCCCCCCCC] |
| Me | 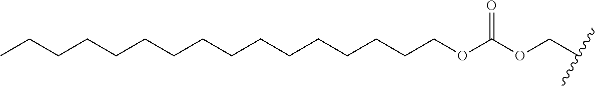 [SMILES: *COC(=O)OCCCCCCCCCCCCCCCC] |
| —C₇H₁₅ | 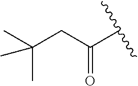 [SMILES: *C(=O)CC(C)(C)C] |
| —C₇H₁₅ | 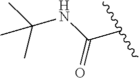 [SMILES: *C(=O)NC(C)(C)C] |
| —C₇H₁₅ | 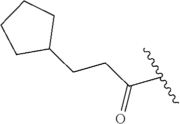 [SMILES: *C(=O)CCC1CCCC1] |
| —C₇H₁₅ | 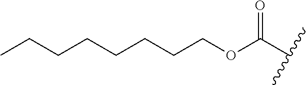 [SMILES: *C(=O)OCCCCCCCC] |
| —C₇H₁₅ | 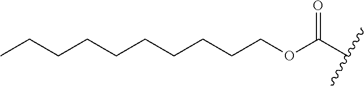 [SMILES: *C(=O)OCCCCCCCCCC] |
| —C₇H₁₅ | 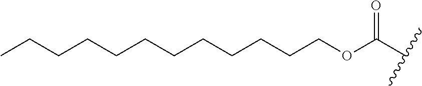 [SMILES: *C(=O)OCCCCCCCCCCCC] |
| —C₇H₁₅ | 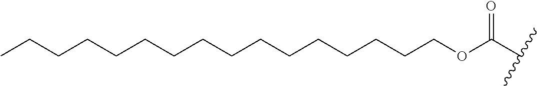 [SMILES: *C(=O)OCCCCCCCCCCCCCCCC] |
| —C₇H₁₅ | 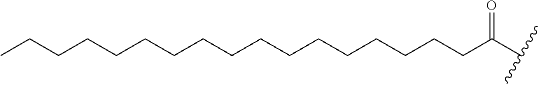 [SMILES: *C(=O)CCCCCCCCCCCCCCCCC] |
| —C₇H₁₅ | 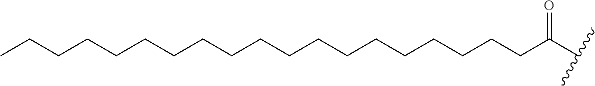 [SMILES: *C(=O)CCCCCCCCCCCCCCCCCCC] |
| —C₇H₁₅ | 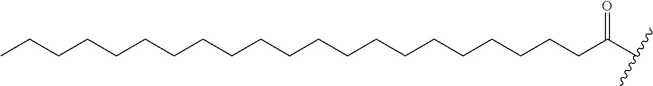 [SMILES: *C(=O)CCCCCCCCCCCCCCCCCCCCC] |
| —C₇H₁₅ | 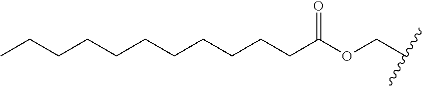 [SMILES: *COC(=O)CCCCCCCCCCC] |
| —C₇H₁₅ | 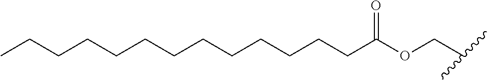 [SMILES: *COC(=O)CCCCCCCCCCCCC] |
| —C₇H₁₅ | 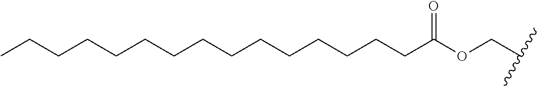 [SMILES: *COC(=O)CCCCCCCCCCCCCCC] |
| —C₇H₁₅ | 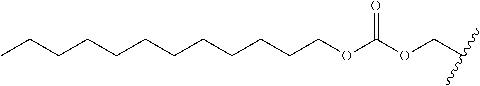 [SMILES: *COC(=O)OCCCCCCCCCCCC] |
| —C₇H₁₅ | 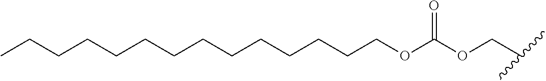 [SMILES: *COC(=O)OCCCCCCCCCCCCCC] |
| —C₇H₁₅ | 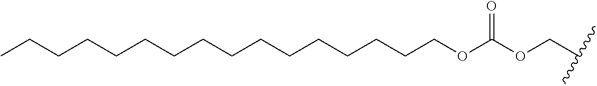 [SMILES: *COC(=O)OCCCCCCCCCCCCCCCC] |
| —C₉H₁₉ | 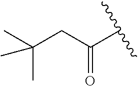 [SMILES: *C(=O)CC(C)(C)C] |
| —C₉H₁₉ | 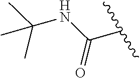 [SMILES: *C(=O)NC(C)(C)C] |
| —C₉H₁₉ | 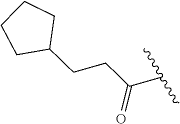 [SMILES: *C(=O)CCC1CCCC1] |
| —C₉H₁₉ | 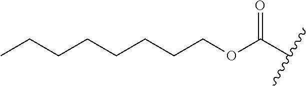 [SMILES: *C(=O)OCCCCCCCC] |
| —C₉H₁₉ | 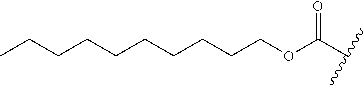 [SMILES: *C(=O)OCCCCCCCCCC] |
| —C₉H₁₉ | 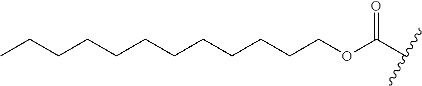 [SMILES: *C(=O)OCCCCCCCCCCCC] |
| —C₉H₁₉ | 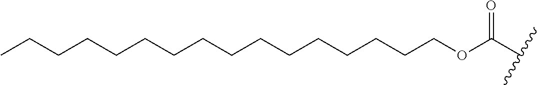 [SMILES: *C(=O)OCCCCCCCCCCCCCCCC] |
| —C₉H₁₉ | 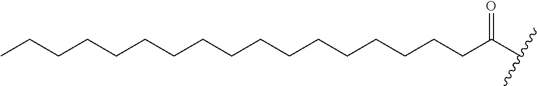 [SMILES: *C(=O)CCCCCCCCCCCCCCCCC] |
| —C₉H₁₉ | 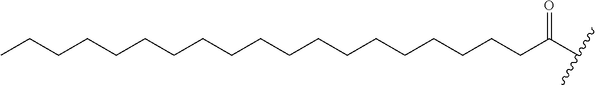 [SMILES: *C(=O)CCCCCCCCCCCCCCCCCCC] |
| —C₉H₁₉ | 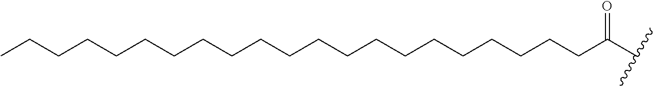 [SMILES: *C(=O)CCCCCCCCCCCCCCCCCCCCC] |
| —C₉H₁₉ | 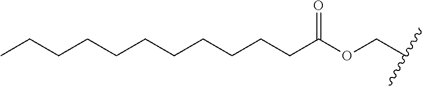 [SMILES: *COC(=O)CCCCCCCCCCC] |
| —C₉H₁₉ | 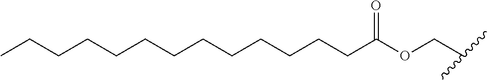 [SMILES: *COC(=O)CCCCCCCCCCCCC] |
| —C₉H₁₉ | 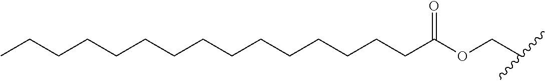 [SMILES: *COC(=O)CCCCCCCCCCCCCCC] |
| —C₉H₁₉ | 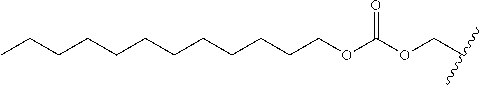 [SMILES: *COC(=O)OCCCCCCCCCCCC] |
| —C₉H₁₉ | 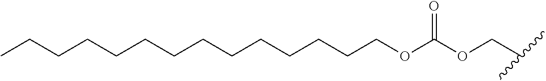 [SMILES: *COC(=O)OCCCCCCCCCCCCCC] |
| —C₉H₁₉ | 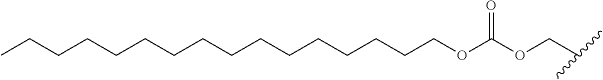 [SMILES: *COC(=O)OCCCCCCCCCCCCCCCC] |
| —C₁₁H₂₃ | 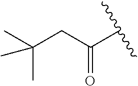 [SMILES: *C(=O)CC(C)(C)C] |
| —C₁₁H₂₃ | 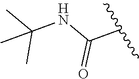 [SMILES: *C(=O)NC(C)(C)C] |
| —C₁₁H₂₃ | 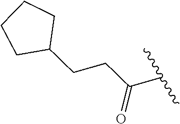 [SMILES: *C(=O)CCC1CCCC1] |
| —C₁₁H₂₃ | 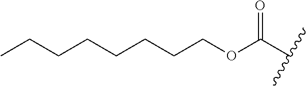 [SMILES: *C(=O)OCCCCCCCC] |
| —C₁₁H₂₃ | 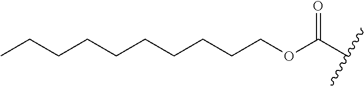 [SMILES: *C(=O)OCCCCCCCCCC] |
| —C₁₁H₂₃ | 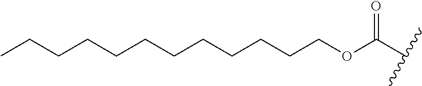 [SMILES: *C(=O)OCCCCCCCCCCCC] |
| —C₁₁H₂₃ | 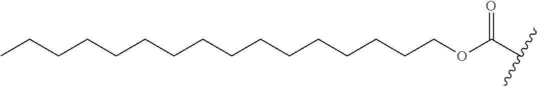 [SMILES: *C(=O)OCCCCCCCCCCCCCCCC] |
| —C₁₁H₂₃ | 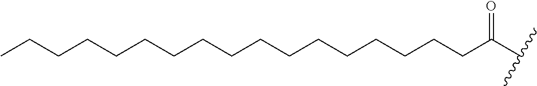 [SMILES: *C(=O)CCCCCCCCCCCCCCCCC] |
| —C₁₁H₂₃ | 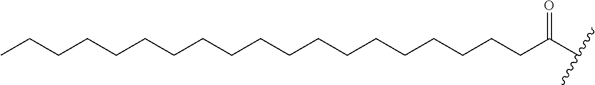 [SMILES: *C(=O)CCCCCCCCCCCCCCCCCCC] |
| —C₁₁H₂₃ | 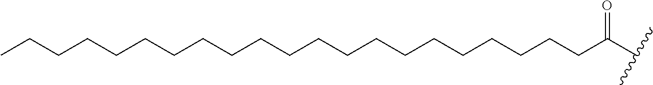 [SMILES: *C(=O)CCCCCCCCCCCCCCCCCCCCC] |
| —C₁₁H₂₃ | 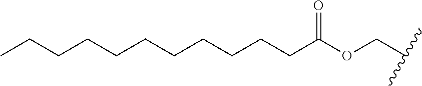 [SMILES: *COC(=O)CCCCCCCCCCC] |
| —C₁₁H₂₃ | 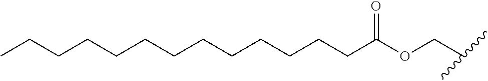 [SMILES: *COC(=O)CCCCCCCCCCCCC] |
| —C₁₁H₂₃ | 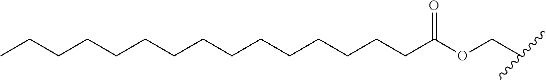 [SMILES: *COC(=O)CCCCCCCCCCCCCCC] |
| —C₁₁H₂₃ | 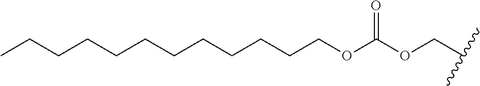 [SMILES: *COC(=O)OCCCCCCCCCCCC] |
| —C₁₁H₂₃ | 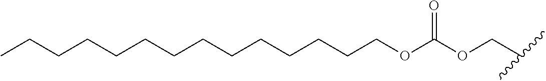 [SMILES: *COC(=O)OCCCCCCCCCCCCCC] |
| —C₁₁H₂₃ | 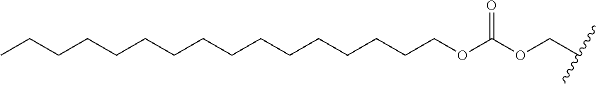 [SMILES: *COC(=O)OCCCCCCCCCCCCCCCC] |
| —C₁₃H₂₇ | 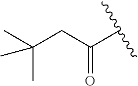 [SMILES: *C(=O)CC(C)(C)C] |
| —C₁₃H₂₇ | 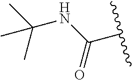 [SMILES: *C(=O)NC(C)(C)C] |
| —C₁₃H₂₇ | 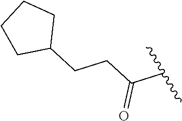 [SMILES: *C(=O)CCC1CCCC1] |
| —C₁₃H₂₇ | 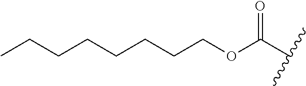 [SMILES: *C(=O)OCCCCCCCC] |
| —C₁₃H₂₇ | 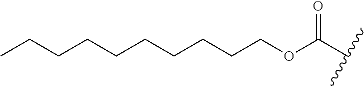 [SMILES: *C(=O)OCCCCCCCCCC] |
| —C₁₃H₂₇ | 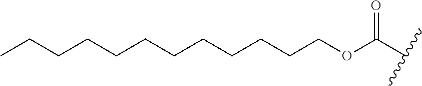 [SMILES: *C(=O)OCCCCCCCCCCCC] |
| —C₁₃H₂₇ | 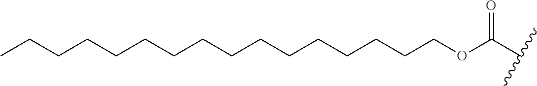 [SMILES: *C(=O)OCCCCCCCCCCCCCCCC] |
| —C₁₃H₂₇ | 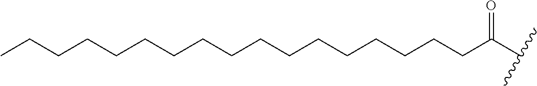 [SMILES: *C(=O)CCCCCCCCCCCCCCCCC] |
| —C₁₃H₂₇ | 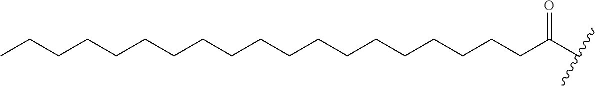 [SMILES: *C(=O)CCCCCCCCCCCCCCCCCCC] |
| —C₁₃H₂₇ | 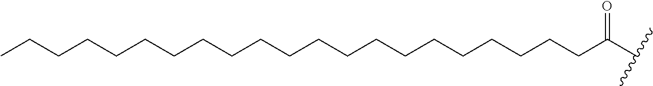 [SMILES: *C(=O)CCCCCCCCCCCCCCCCCCCCC] |
| —C₁₃H₂₇ | 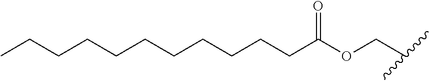 [SMILES: *COC(=O)CCCCCCCCCCC] |
| —C₁₃H₂₇ | 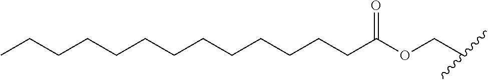 [SMILES: *COC(=O)CCCCCCCCCCCCC] |
| —C₁₃H₂₇ | 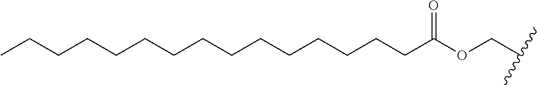 [SMILES: *COC(=O)CCCCCCCCCCCCCCC] |
| —C₁₃H₂₇ | 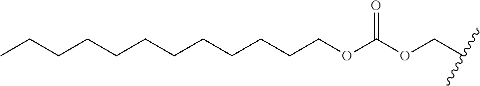 [SMILES: *COC(=O)OCCCCCCCCCCCC] |
| —C₁₃H₂₇ | 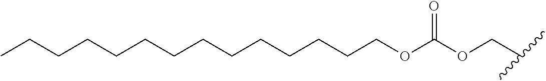 [SMILES: *COC(=O)OCCCCCCCCCCCCCC] |
| —C₁₃H₂₇ | 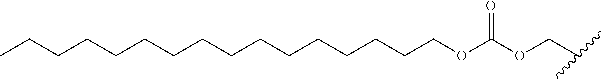 [SMILES: *COC(=O)OCCCCCCCCCCCCCCCC] |
| —C₁₅H₃₁ | 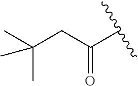 [SMILES: *C(=O)CC(C)(C)C] |
| —C₁₅H₃₁ | 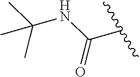 [SMILES: *C(=O)NC(C)(C)C] |
| —C₁₅H₃₁ | 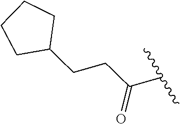 [SMILES: *C(=O)CCC1CCCC1] |
| —C₁₅H₃₁ | 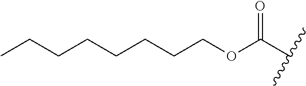 [SMILES: *C(=O)OCCCCCCCC] |
| —C₁₅H₃₁ | 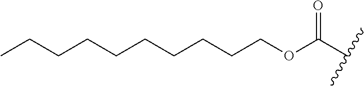 [SMILES: *C(=O)OCCCCCCCCCC] |
| —C₁₅H₃₁ | 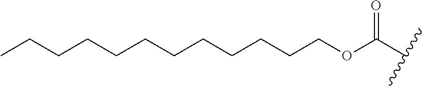 [SMILES: *C(=O)OCCCCCCCCCCCC] |
| —C₁₅H₃₁ | 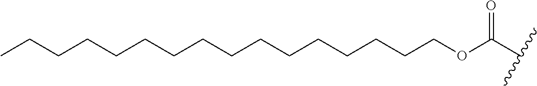 [SMILES: *C(=O)OCCCCCCCCCCCCCCCC] |
| —C₁₅H₃₁ | 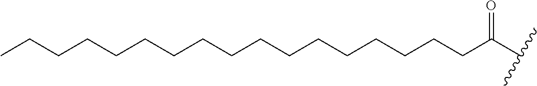 [SMILES: *C(=O)CCCCCCCCCCCCCCCCC] |
| —C₁₅H₃₁ | 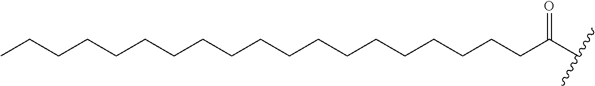 [SMILES: *C(=O)CCCCCCCCCCCCCCCCCCC] |
| —C₁₅H₃₁ | 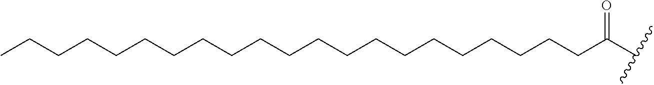 [SMILES: *C(=O)CCCCCCCCCCCCCCCCCCCCC] |
| —C₁₅H₃₁ | 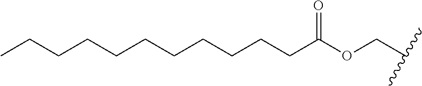 [SMILES: *COC(=O)CCCCCCCCCCC] |
| —C₁₅H₃₁ | 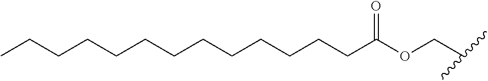 [SMILES: *COC(=O)CCCCCCCCCCCCC] |
| —C₁₅H₃₁ | 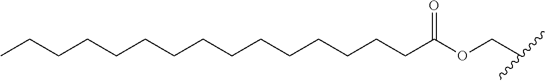 [SMILES: *COC(=O)CCCCCCCCCCCCCCC] |
| —C₁₅H₃₁ | 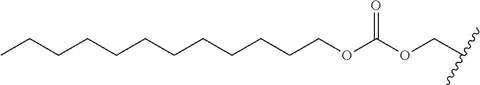 [SMILES: *COC(=O)OCCCCCCCCCCCC] |
| —C₁₅H₃₁ | 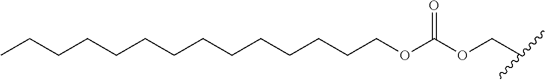 [SMILES: *COC(=O)OCCCCCCCCCCCCCC] |
| —C₁₅H₃₁ | 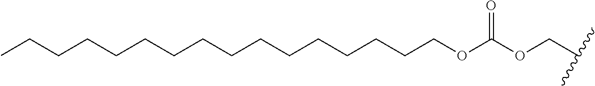 [SMILES: *COC(=O)OCCCCCCCCCCCCCCCC] |
| —C₁₇H₃₅ | 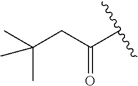 [SMILES: *C(=O)CC(C)(C)C] |
| —C₁₇H₃₅ | 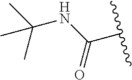 [SMILES: *C(=O)NC(C)(C)C] |
| —C₁₇H₃₅ | 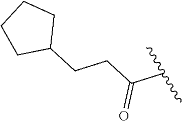 [SMILES: *C(=O)CCC1CCCC1] |
| —C₁₇H₃₅ | 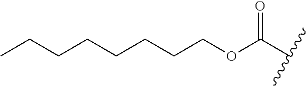 [SMILES: *C(=O)OCCCCCCCC] |
| —C₁₇H₃₅ | 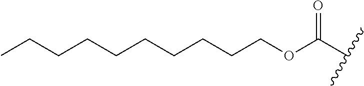 [SMILES: *C(=O)OCCCCCCCCCC] |
| —C₁₇H₃₅ | 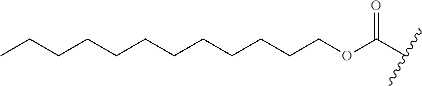 [SMILES: *C(=O)OCCCCCCCCCCCC] |
| —C₁₇H₃₅ | 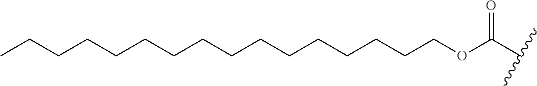 [SMILES: *C(=O)OCCCCCCCCCCCCCCCC] |
| —C₁₇H₃₅ | 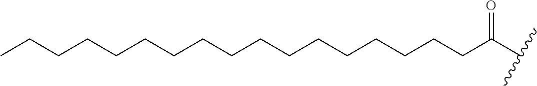 [SMILES: *C(=O)CCCCCCCCCCCCCCCCC] |
| —C₁₇H₃₅ | 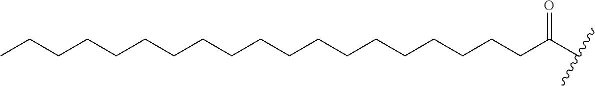 [SMILES: *C(=O)CCCCCCCCCCCCCCCCCCC] |
| —C₁₇H₃₅ | 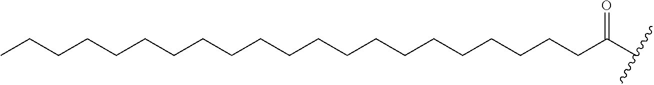 [SMILES: *C(=O)CCCCCCCCCCCCCCCCCCCCC] |
| —C₁₇H₃₅ | 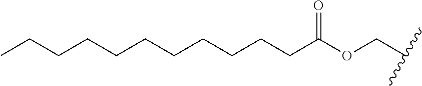 [SMILES: *COC(=O)CCCCCCCCCCC] |
| —C₁₇H₃₅ | 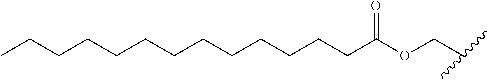 [SMILES: *COC(=O)CCCCCCCCCCCCC] |
| —C₁₇H₃₅ | 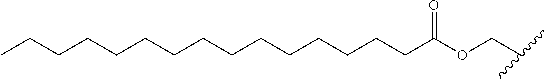 [SMILES: *COC(=O)CCCCCCCCCCCCCCC] |
| —C₁₇H₃₅ | 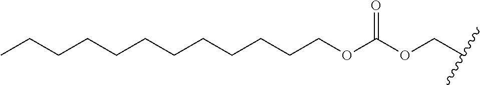 [SMILES: *COC(=O)OCCCCCCCCCCCC] |
| —C₁₇H₃₅ | 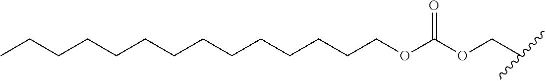 [SMILES: *COC(=O)OCCCCCCCCCCCCCC] |
| —C₁₇H₃₅ | 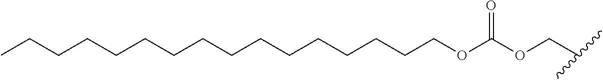 [SMILES: *COC(=O)OCCCCCCCCCCCCCCCC] |
| 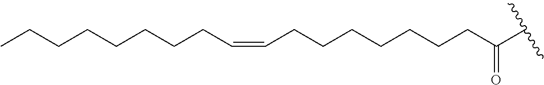 [SMILES: *C(=O)CCCCCCC/C=C\CCCCCCCC] | 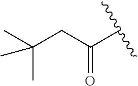 [SMILES: *C(=O)CC(C)(C)C] |
| 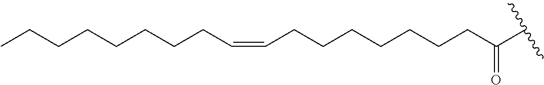 [SMILES: *C(=O)CCCCCCC/C=C\CCCCCCCC] | 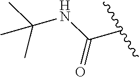 [SMILES: *C(=O)NC(C)(C)C] |
| 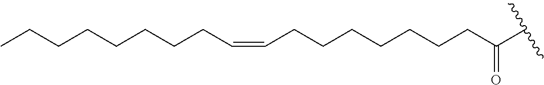 [SMILES: *C(=O)CCCCCCC/C=C\CCCCCCCC] | 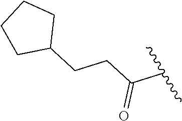 [SMILES: *C(=O)CCC1CCCC1] |
| 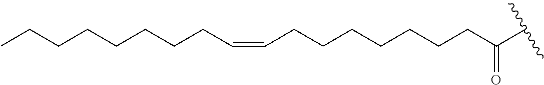 [SMILES: *C(=O)CCCCCCC/C=C\CCCCCCCC] | 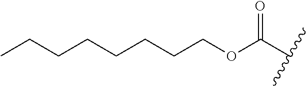 [SMILES: *C(=O)OCCCCCCCC] |
| 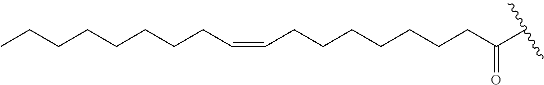 [SMILES: *C(=O)CCCCCCC/C=C\CCCCCCCC] | 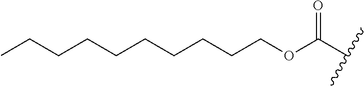 [SMILES: *C(=O)OCCCCCCCCCC] |
| 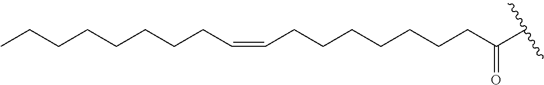 [SMILES: *C(=O)CCCCCCC/C=C\CCCCCCCC] | 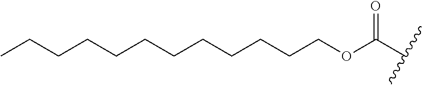 [SMILES: *C(=O)OCCCCCCCCCCCC] |
| 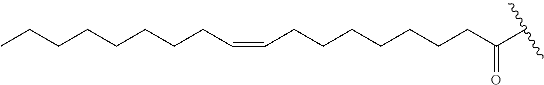 [SMILES: *C(=O)CCCCCCC/C=C\CCCCCCCC] | 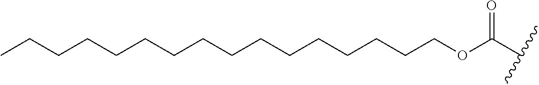 [SMILES: *C(=O)OCCCCCCCCCCCCCCCC] |
| 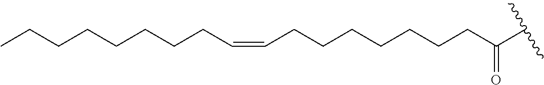 [SMILES: *C(=O)CCCCCCC/C=C\CCCCCCCC] | 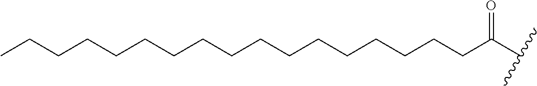 [SMILES: *C(=O)CCCCCCCCCCCCCCCCC] |
| 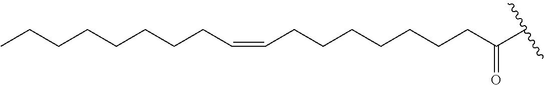 [SMILES: *C(=O)CCCCCCC/C=C\CCCCCCCC] | 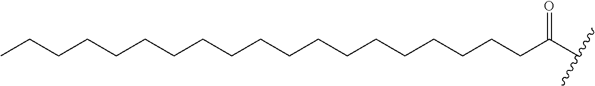 [SMILES: *C(=O)CCCCCCCCCCCCCCCCCCC] |
| 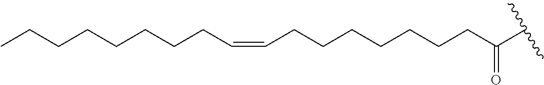 [SMILES: *C(=O)CCCCCCC/C=C\CCCCCCCC] | 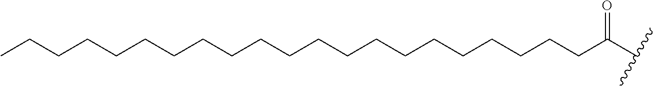 [SMILES: *C(=O)CCCCCCCCCCCCCCCCCCCCC] |
| 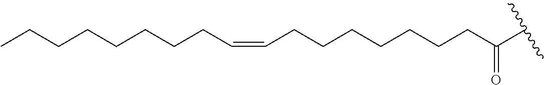 [SMILES: *C(=O)CCCCCCC/C=C\CCCCCCCC] | 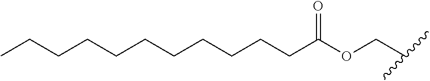 [SMILES: *COC(=O)CCCCCCCCCCC] |
| 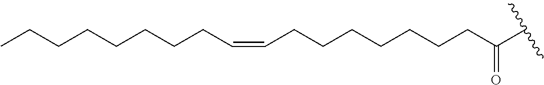 [SMILES: *C(=O)CCCCCCC/C=C\CCCCCCCC] | 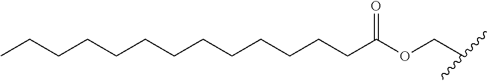 [SMILES: *COC(=O)CCCCCCCCCCCCC] |
| 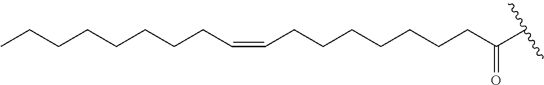 [SMILES: *C(=O)CCCCCCC/C=C\CCCCCCCC] | 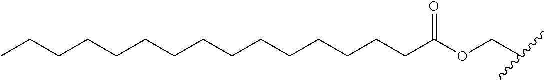 [SMILES: *COC(=O)CCCCCCCCCCCCCCC] |
| 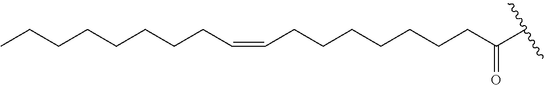 [SMILES: *C(=O)CCCCCCC/C=C\CCCCCCCC] | 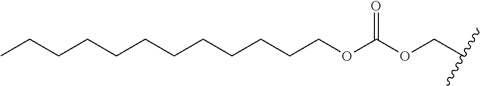 [SMILES: *COC(=O)OCCCCCCCCCCCC] |
| 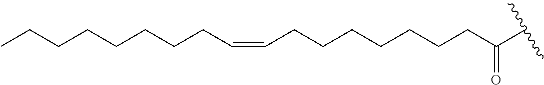 [SMILES: *C(=O)CCCCCCC/C=C\CCCCCCCC] | 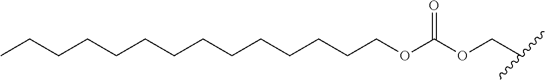 [SMILES: *COC(=O)OCCCCCCCCCCCCCC] |
| 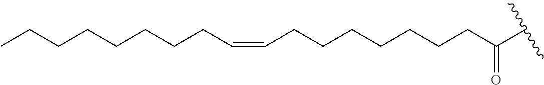 [SMILES: *C(=O)CCCCCCC/C=C\CCCCCCCC] | 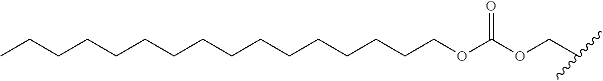 [SMILES: *COC(=O)OCCCCCCCCCCCCCCCC] |
| 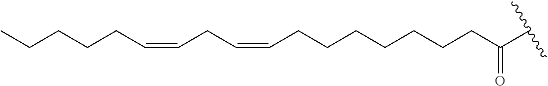 [SMILES: *C(=O)CCCCCCC/C=C\C/C=C\CCCCC] | 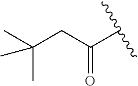 [SMILES: *C(=O)CC(C)(C)C] |
| 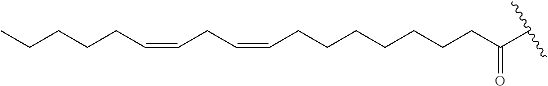 [SMILES: *C(=O)CCCCCCC/C=C\C/C=C\CCCCC] | 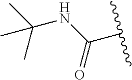 [SMILES: *C(=O)NC(C)(C)C] |
| 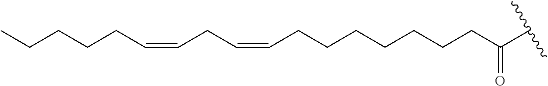 [SMILES: *C(=O)CCCCCCC/C=C\C/C=C\CCCCC] | 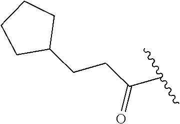 [SMILES: *C(=O)CCC1CCCC1] |
| 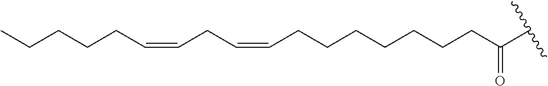 [SMILES: *C(=O)CCCCCCC/C=C\C/C=C\CCCCC] | 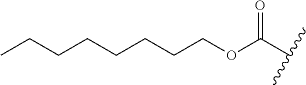 [SMILES: *C(=O)OCCCCCCCC] |
| 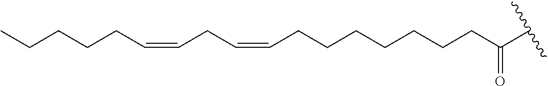 [SMILES: *C(=O)CCCCCCC/C=C\C/C=C\CCCCC] | 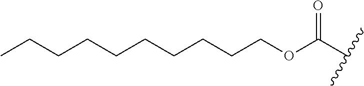 [SMILES: *C(=O)OCCCCCCCCCC] |
| 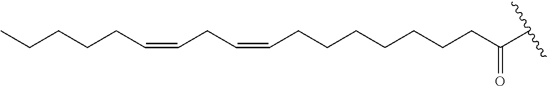 [SMILES: *C(=O)CCCCCCC/C=C\C/C=C\CCCCC] | 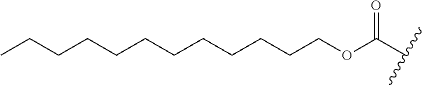 [SMILES: *C(=O)OCCCCCCCCCCCC] |
| 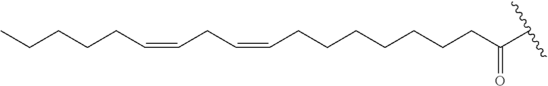 [SMILES: *C(=O)CCCCCCC/C=C\C/C=C\CCCCC] | 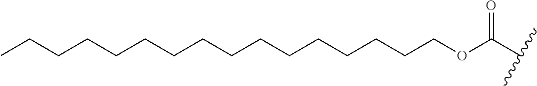 [SMILES: *C(=O)OCCCCCCCCCCCCCCCC] |
| 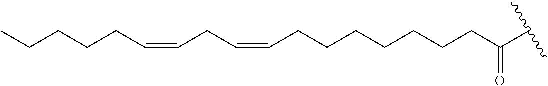 [SMILES: *C(=O)CCCCCCC/C=C\C/C=C\CCCCC] | 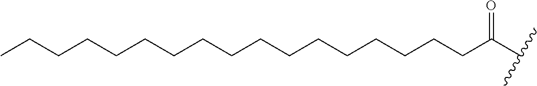 [SMILES: *C(=O)CCCCCCCCCCCCCCCCC] |
| 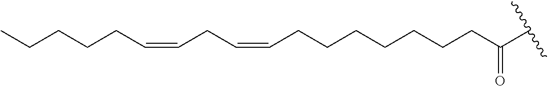 [SMILES: *C(=O)CCCCCCC/C=C\C/C=C\CCCCC] | 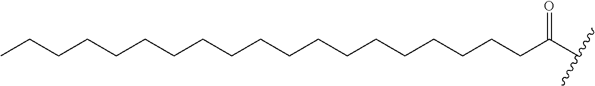 [SMILES: *C(=O)CCCCCCCCCCCCCCCCCCC] |
| 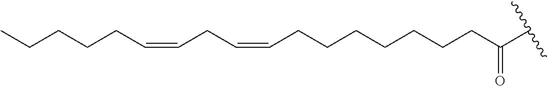 [SMILES: *C(=O)CCCCCCC/C=C\C/C=C\CCCCC] | 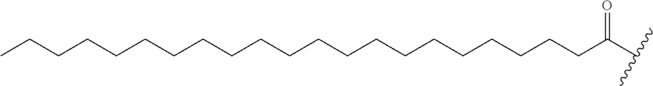 [SMILES: *C(=O)CCCCCCCCCCCCCCCCCCCCC] |
| 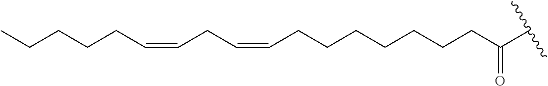 [SMILES: *C(=O)CCCCCCC/C=C\C/C=C\CCCCC] | 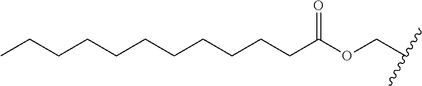 [SMILES: *COC(=O)CCCCCCCCCCC] |
| 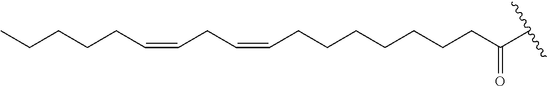 [SMILES: *C(=O)CCCCCCC/C=C\C/C=C\CCCCC] | 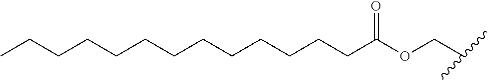 [SMILES: *COC(=O)CCCCCCCCCCCCC] |
| 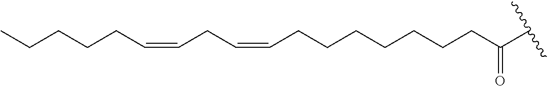 [SMILES: *C(=O)CCCCCCC/C=C\C/C=C\CCCCC] | 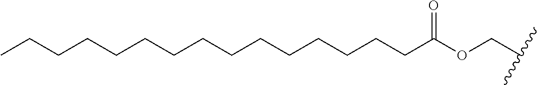 [SMILES: *COC(=O)CCCCCCCCCCCCCCC] |
| 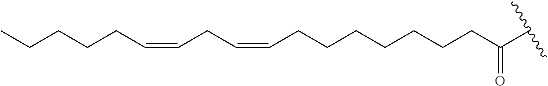 [SMILES: *C(=O)CCCCCCC/C=C\C/C=C\CCCCC] | 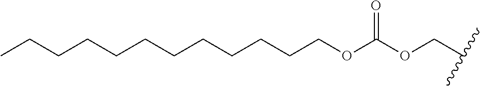 [SMILES: *COC(=O)OCCCCCCCCCCCC] |
| 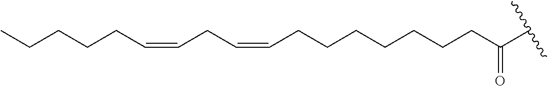 [SMILES: *C(=O)CCCCCCC/C=C\C/C=C\CCCCC] | 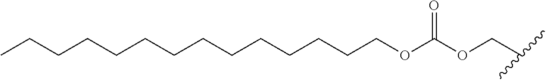 [SMILES: *COC(=O)OCCCCCCCCCCCCCC] |
| 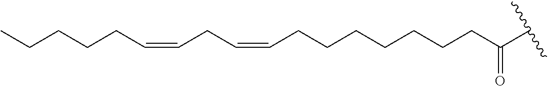 [SMILES: *C(=O)CCCCCCC/C=C\C/C=C\CCCCC] | 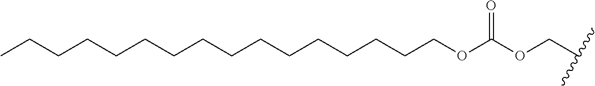 [SMILES: *COC(=O)OCCCCCCCCCCCCCCCC] |
| 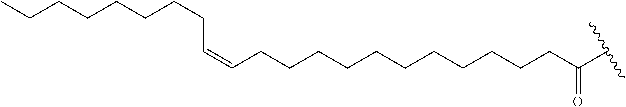 [SMILES: *C(=O)CCCCCCCCCCC/C=C\CCCCCCCC] | 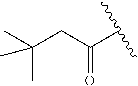 [SMILES: *C(=O)CC(C)(C)C] |
| 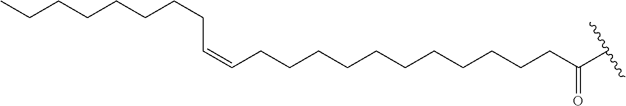 [SMILES: *C(=O)CCCCCCCCCCC/C=C\CCCCCCCC] | 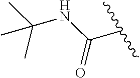 [SMILES: *C(=O)NC(C)(C)C] |
| 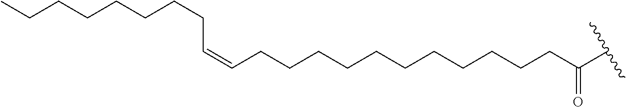 [SMILES: *C(=O)CCCCCCCCCCC/C=C\CCCCCCCC] | 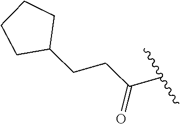 [SMILES: *C(=O)CCC1CCCC1] |
| 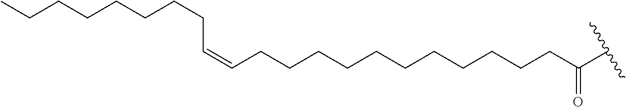 [SMILES: *C(=O)CCCCCCCCCCC/C=C\CCCCCCCC] | 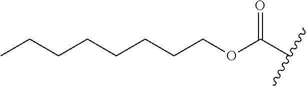 [SMILES: *C(=O)OCCCCCCCC] |
| 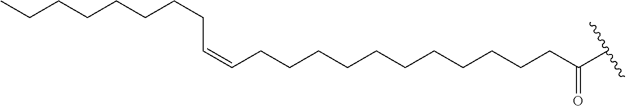 [SMILES: *C(=O)CCCCCCCCCCC/C=C\CCCCCCCC] | 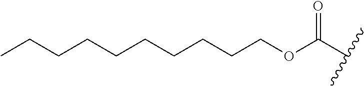 [SMILES: *C(=O)OCCCCCCCCCC] |
| 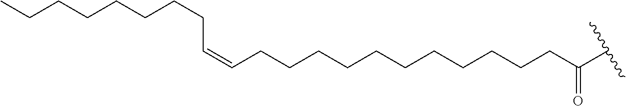 [SMILES: *C(=O)CCCCCCCCCCC/C=C\CCCCCCCC] | 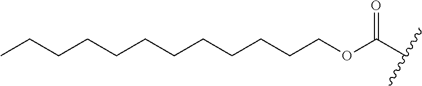 [SMILES: *C(=O)OCCCCCCCCCCCC] |
| 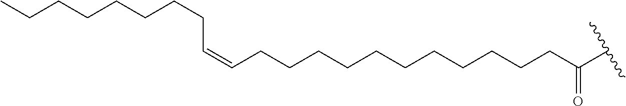 [SMILES: *C(=O)CCCCCCCCCCC/C=C\CCCCCCCC] | 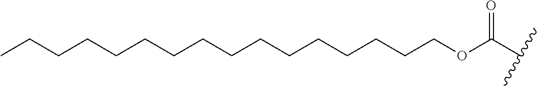 [SMILES: *C(=O)OCCCCCCCCCCCCCCCC] |
| 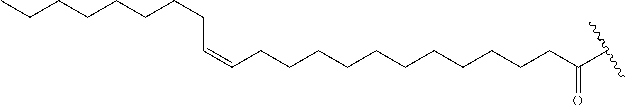 [SMILES: *C(=O)CCCCCCCCCCC/C=C\CCCCCCCC] | 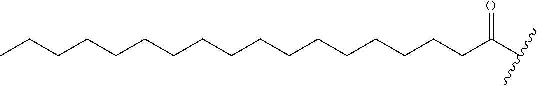 [SMILES: *C(=O)CCCCCCCCCCCCCCCCC] |
| 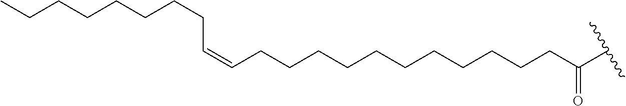 [SMILES: *C(=O)CCCCCCCCCCC/C=C\CCCCCCCC] | 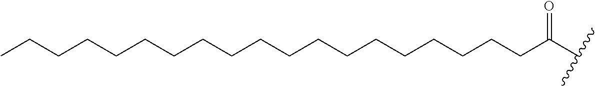 [SMILES: *C(=O)CCCCCCCCCCCCCCCCCCC] |
| 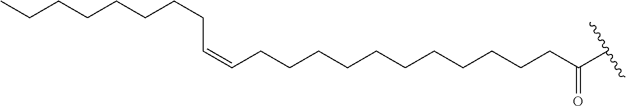 [SMILES: *C(=O)CCCCCCCCCCC/C=C\CCCCCCCC] | 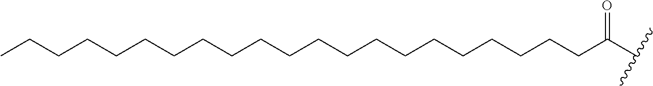 [SMILES: *C(=O)CCCCCCCCCCCCCCCCCCCCC] |
| 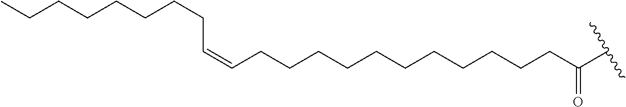 [SMILES: *C(=O)CCCCCCCCCCC/C=C\CCCCCCCC] | 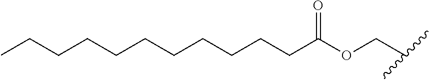 [SMILES: *COC(=O)CCCCCCCCCCC] |
| 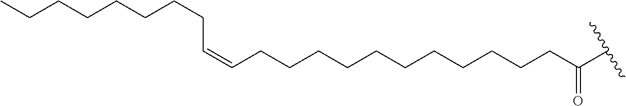 [SMILES: *C(=O)CCCCCCCCCCC/C=C\CCCCCCCC] | 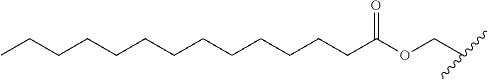 [SMILES: *COC(=O)CCCCCCCCCCCCC] |
| 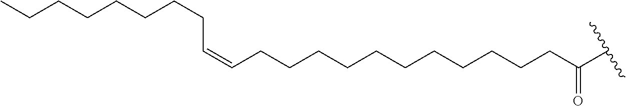 [SMILES: *C(=O)CCCCCCCCCCC/C=C\CCCCCCCC] | 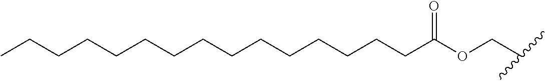 [SMILES: *COC(=O)CCCCCCCCCCCCCCC] |
| 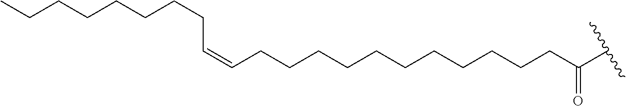 [SMILES: *C(=O)CCCCCCCCCCC/C=C\CCCCCCCC] | 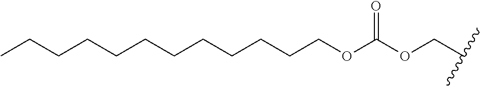 [SMILES: *COC(=O)OCCCCCCCCCCCC] |
| 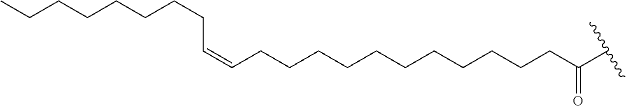 [SMILES: *C(=O)CCCCCCCCCCC/C=C\CCCCCCCC] | 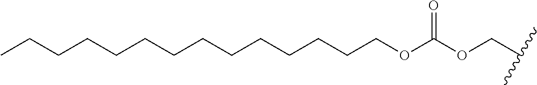 [SMILES: *COC(=O)OCCCCCCCCCCCCCC] |
| 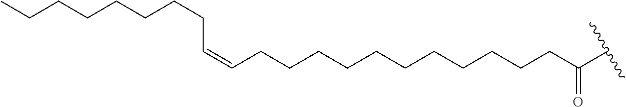 [SMILES: *C(=O)CCCCCCCCCCC/C=C\CCCCCCCC] | 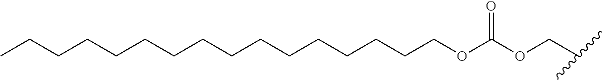 [SMILES: *COC(=O)OCCCCCCCCCCCCCCCC] |
| 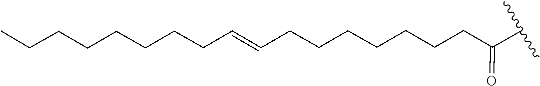 [SMILES: *C(=O)CCCCCCC/C=C/CCCCCCCC] | 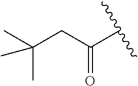 [SMILES: *C(=O)CC(C)(C)C] |
| 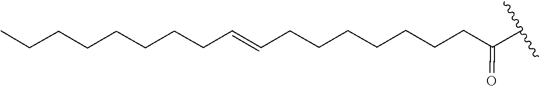 [SMILES: *C(=O)CCCCCCC/C=C/CCCCCCCC] | 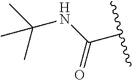 [SMILES: *C(=O)NC(C)(C)C] |
| 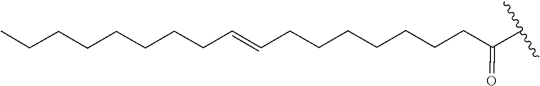 [SMILES: *C(=O)CCCCCCC/C=C/CCCCCCCC] | 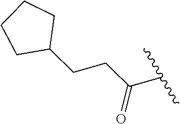 [SMILES: *C(=O)CCC1CCCC1] |
| 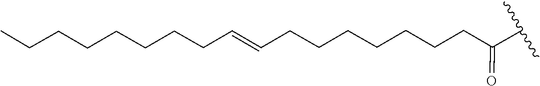 [SMILES: *C(=O)CCCCCCC/C=C/CCCCCCCC] | 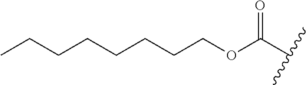 [SMILES: *C(=O)OCCCCCCCC] |
| 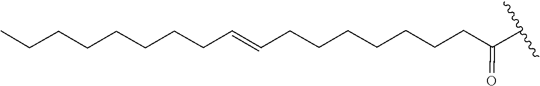 [SMILES: *C(=O)CCCCCCC/C=C/CCCCCCCC] | 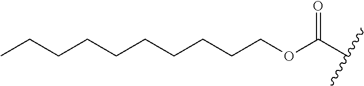 [SMILES: *C(=O)OCCCCCCCCCC] |
| 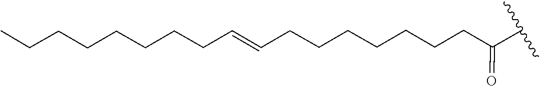 [SMILES: *C(=O)CCCCCCC/C=C/CCCCCCCC] | 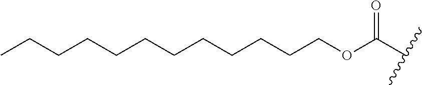 [SMILES: *C(=O)OCCCCCCCCCCCC] |
| 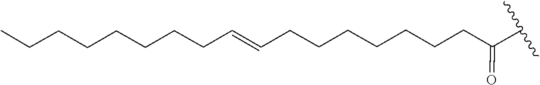 [SMILES: *C(=O)CCCCCCC/C=C/CCCCCCCC] | 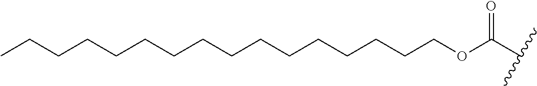 [SMILES: *C(=O)OCCCCCCCCCCCCCCCC] |
| 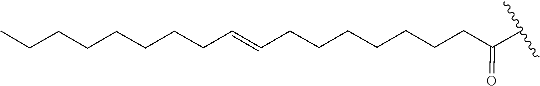 [SMILES: *C(=O)CCCCCCC/C=C/CCCCCCCC] | 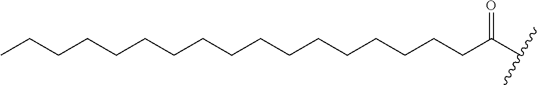 [SMILES: *C(=O)CCCCCCCCCCCCCCCCC] |
| 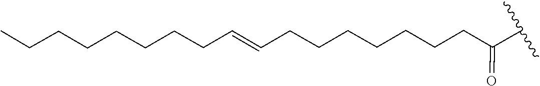 [SMILES: *C(=O)CCCCCCC/C=C/CCCCCCCC] | 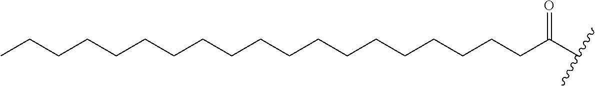 [SMILES: *C(=O)CCCCCCCCCCCCCCCCCCC] |
| 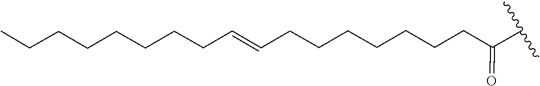 [SMILES: *C(=O)CCCCCCC/C=C/CCCCCCCC] | 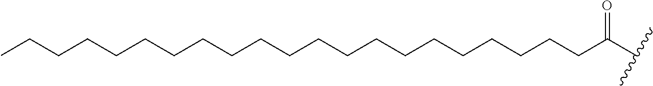 [SMILES: *C(=O)CCCCCCCCCCCCCCCCCCCCC] |
| 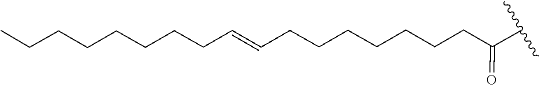 [SMILES: *C(=O)CCCCCCC/C=C/CCCCCCCC] | 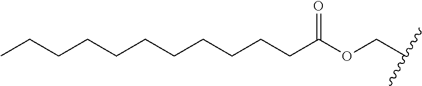 [SMILES: *COC(=O)CCCCCCCCCCC] |
| 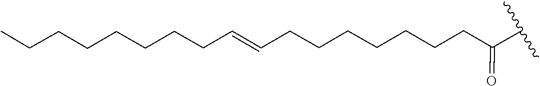 [SMILES: *C(=O)CCCCCCC/C=C/CCCCCCCC] | 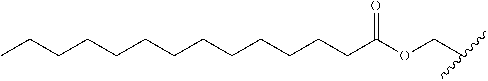 [SMILES: *COC(=O)CCCCCCCCCCCCC] |
| 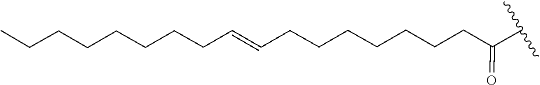 [SMILES: *C(=O)CCCCCCC/C=C/CCCCCCCC] | 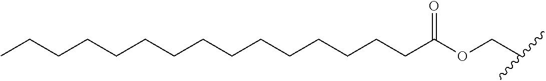 [SMILES: *COC(=O)CCCCCCCCCCCCCCC] |
| 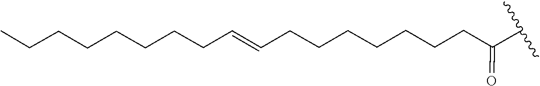 [SMILES: *C(=O)CCCCCCC/C=C/CCCCCCCC] | 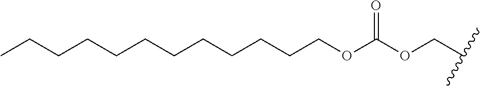 [SMILES: *COC(=O)OCCCCCCCCCCCC] |
| 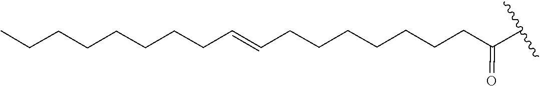 [SMILES: *C(=O)CCCCCCC/C=C/CCCCCCCC] | 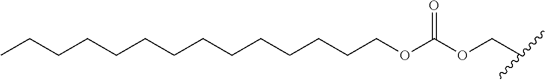 [SMILES: *COC(=O)OCCCCCCCCCCCCCC] |
| 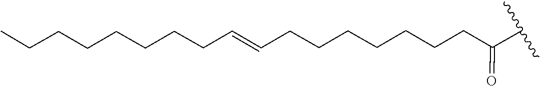 [SMILES: *C(=O)CCCCCCC/C=C/CCCCCCCC] | 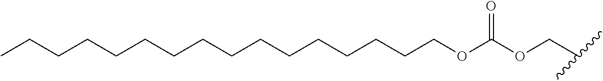 [SMILES: *COC(=O)OCCCCCCCCCCCCCCCC] |
| 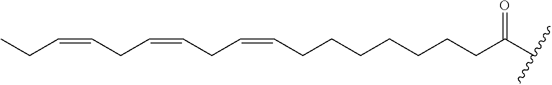 [SMILES: *C(=O)CCCCCCC/C=C\C/C=C\C/C=C\CC] | 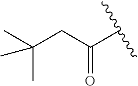 [SMILES: *C(=O)CC(C)(C)C] |
| 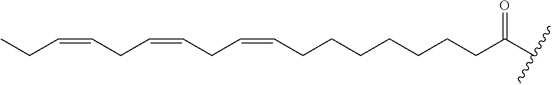 [SMILES: *C(=O)CCCCCCC/C=C\C/C=C\C/C=C\CC] | 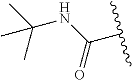 [SMILES: *C(=O)NC(C)(C)C] |
| 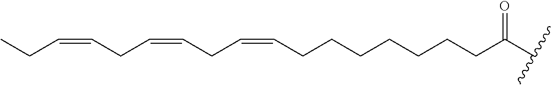 [SMILES: *C(=O)CCCCCCC/C=C\C/C=C\C/C=C\CC] | 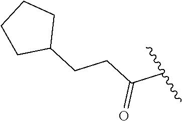 [SMILES: *C(=O)CCC1CCCC1] |
| 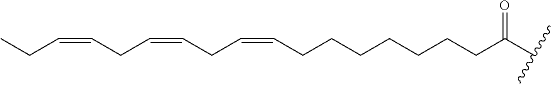 [SMILES: *C(=O)CCCCCCC/C=C\C/C=C\C/C=C\CC] | 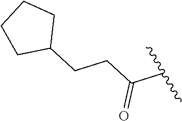 [SMILES: *C(=O)CCC1CCCC1] |
| 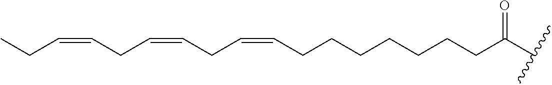 [SMILES: *C(=O)CCCCCCC/C=C\C/C=C\C/C=C\CC] | 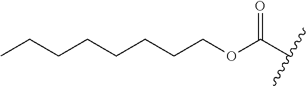 [SMILES: *C(=O)OCCCCCCCC] |
| 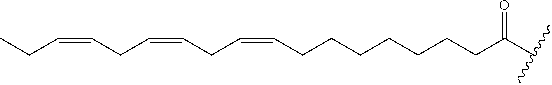 [SMILES: *C(=O)CCCCCCC/C=C\C/C=C\C/C=C\CC] | 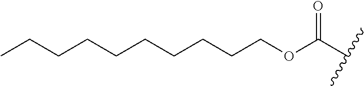 [SMILES: *C(=O)OCCCCCCCCCC] |
| 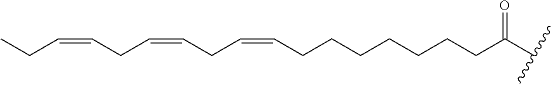 [SMILES: *C(=O)CCCCCCC/C=C\C/C=C\C/C=C\CC] | 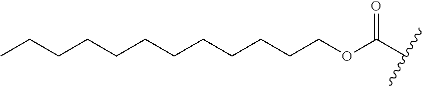 [SMILES: *C(=O)OCCCCCCCCCCCC] |
| 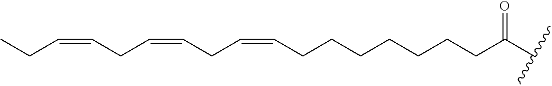 [SMILES: *C(=O)CCCCCCC/C=C\C/C=C\C/C=C\CC] | 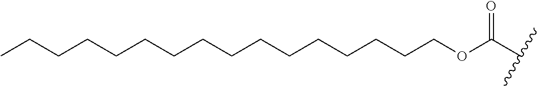 [SMILES: *C(=O)OCCCCCCCCCCCCCCCC] |
| 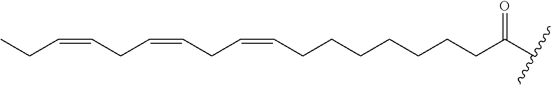 [SMILES: *C(=O)CCCCCCC/C=C\C/C=C\C/C=C\CC] | 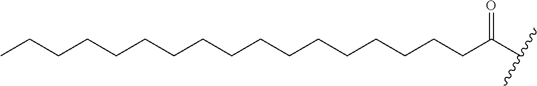 [SMILES: *C(=O)CCCCCCCCCCCCCCCCC] |
| 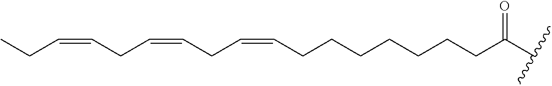 [SMILES: *C(=O)CCCCCCC/C=C\C/C=C\C/C=C\CC] | 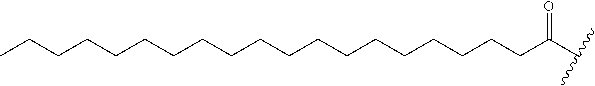 [SMILES: *C(=O)CCCCCCCCCCCCCCCCCCC] |
| 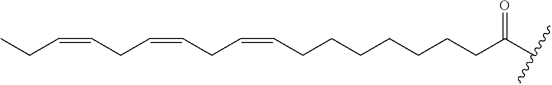 [SMILES: *C(=O)CCCCCCC/C=C\C/C=C\C/C=C\CC] | 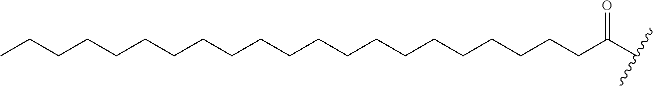 [SMILES: *C(=O)CCCCCCCCCCCCCCCCCCCCC] |
| 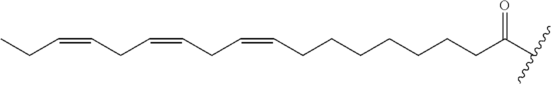 [SMILES: *C(=O)CCCCCCC/C=C\C/C=C\C/C=C\CC] | 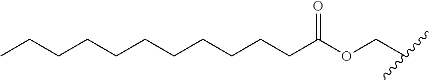 [SMILES: *COC(=O)CCCCCCCCCCC] |
| 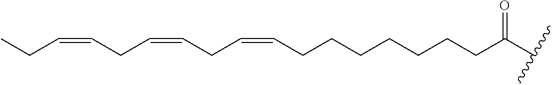 [SMILES: *C(=O)CCCCCCC/C=C\C/C=C\C/C=C\CC] | 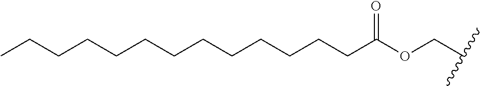 [SMILES: *COC(=O)CCCCCCCCCCCCC] |
| 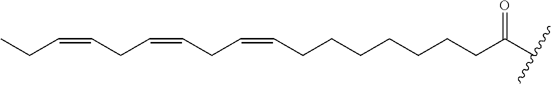 [SMILES: *C(=O)CCCCCCC/C=C\C/C=C\C/C=C\CC] | 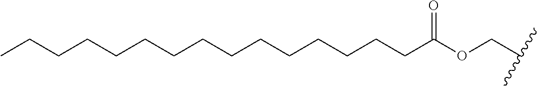 [SMILES: *COC(=O)CCCCCCCCCCCCCCC] |
| 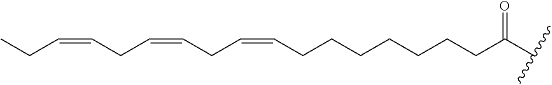 [SMILES: *C(=O)CCCCCCC/C=C\C/C=C\C/C=C\CC] | 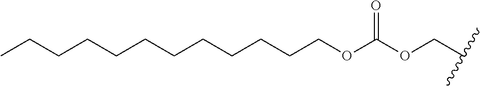 [SMILES: *COC(=O)OCCCCCCCCCCCC] |
| 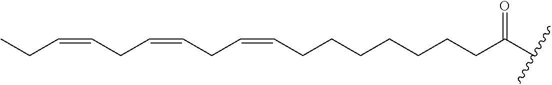 [SMILES: *C(=O)CCCCCCC/C=C\C/C=C\C/C=C\CC] | 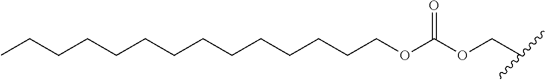 [SMILES: *COC(=O)OCCCCCCCCCCCCCC] |
| 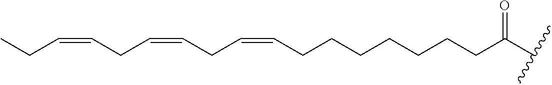 [SMILES: *C(=O)CCCCCCC/C=C\C/C=C\C/C=C\CC] | 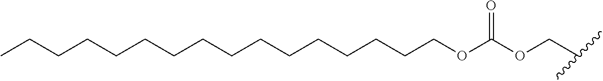 [SMILES: *COC(=O)OCCCCCCCCCCCCCCCC] |
| tBu | 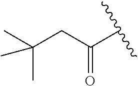 [SMILES: *C(=O)CC(C)(C)C] |
| tBu | 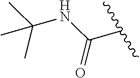 [SMILES: *C(=O)NC(C)(C)C] |
| tBu | 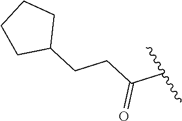 [SMILES: *C(=O)CCC1CCCC1] |
| tBu | 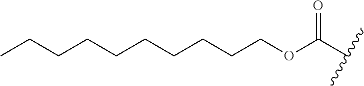 [SMILES: *C(=O)OCCCCCCCCCC] |
| tBu | 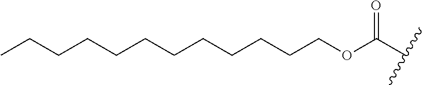 [SMILES: *C(=O)OCCCCCCCCCCCC] |
| tBu | 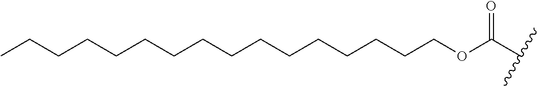 [SMILES: *C(=O)OCCCCCCCCCCCCCCCC] |
| tBu | 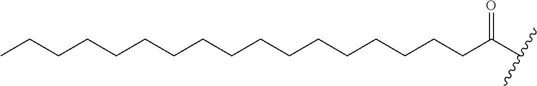 [SMILES: *C(=O)CCCCCCCCCCCCCCCCC] |
| tBu | 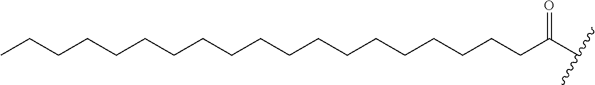 [SMILES: *C(=O)CCCCCCCCCCCCCCCCCCC] |
| tBu | 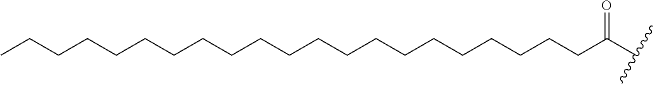 [SMILES: *C(=O)CCCCCCCCCCCCCCCCCCCCC] |
| tBu | 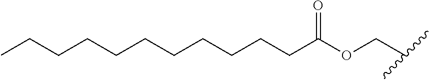 [SMILES: *COC(=O)CCCCCCCCCCC] |
| tBu | 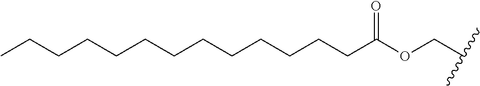 [SMILES: *COC(=O)CCCCCCCCCCCCC] |
| tBu | 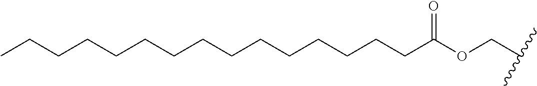 [SMILES: *COC(=O)CCCCCCCCCCCCCCC] |
| tBu | 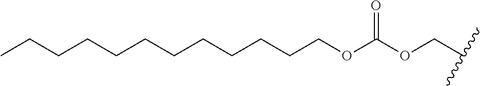 [SMILES: *COC(=O)OCCCCCCCCCCCC] |
| tBu | 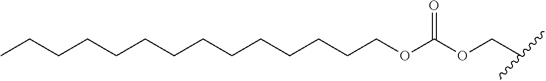 [SMILES: *COC(=O)OCCCCCCCCCCCCCC] |
| tBu | 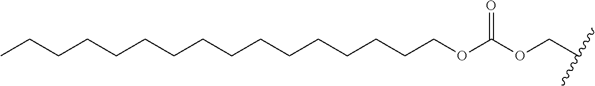 [SMILES: *COC(=O)OCCCCCCCCCCCCCCCC] |
| phenyl | 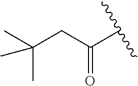 [SMILES: *C(=O)CC(C)(C)C] |
| phenyl | 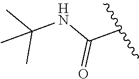 [SMILES: *C(=O)NC(C)(C)C] |
| phenyl | 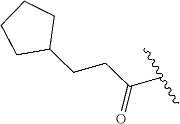 [SMILES: *C(=O)CCC1CCCC1] |
| phenyl | 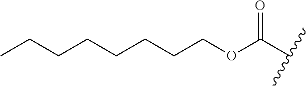 [SMILES: *C(=O)OCCCCCCCC] |
| phenyl | 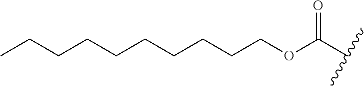 [SMILES: *C(=O)OCCCCCCCCCC] |
| phenyl | 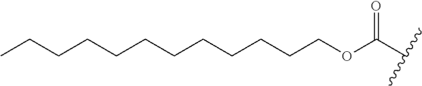 [SMILES: *C(=O)OCCCCCCCCCCCC] |
| phenyl | 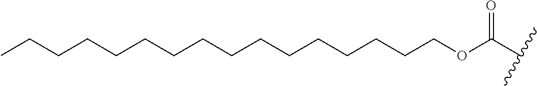 [SMILES: *C(=O)OCCCCCCCCCCCCCCCC] |
| phenyl | 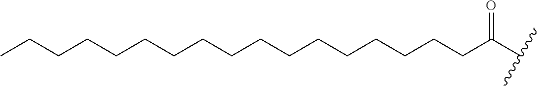 [SMILES: *C(=O)CCCCCCCCCCCCCCCCC] |
| phenyl | 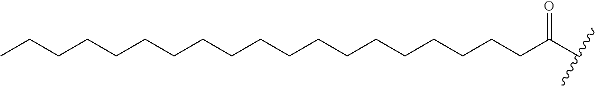 [SMILES: *C(=O)CCCCCCCCCCCCCCCCCCC] |
| phenyl | 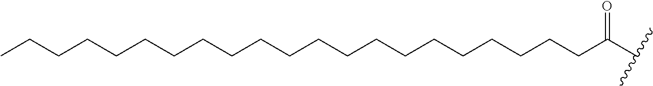 [SMILES: *C(=O)CCCCCCCCCCCCCCCCCCCCC] |
| phenyl | 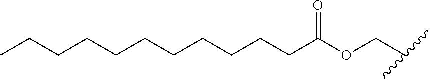 [SMILES: *COC(=O)CCCCCCCCCCC] |
| phenyl | 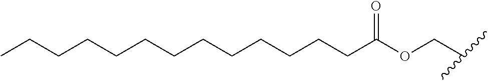 [SMILES: *COC(=O)CCCCCCCCCCCCC] |
| phenyl | 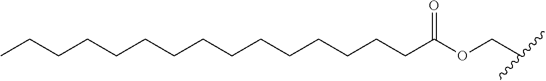 [SMILES: *COC(=O)CCCCCCCCCCCCCCC] |
| phenyl | 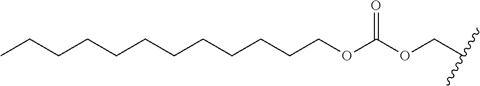 [SMILES: *COC(=O)OCCCCCCCCCCCC] |
| phenyl | 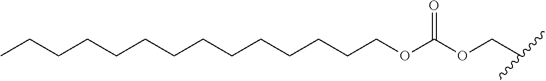 [SMILES: *COC(=O)OCCCCCCCCCCCCCC] |
| phenyl | 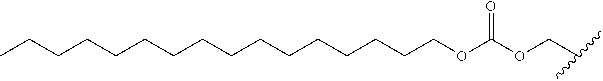 [SMILES: *COC(=O)OCCCCCCCCCCCCCCCC] |
| 4-chlorophenyl | 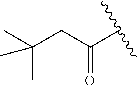 [SMILES: *C(=O)CC(C)(C)C] |
| 4-chlorophenyl | 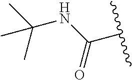 [SMILES: *C(=O)NC(C)(C)C] |
| 4-chlorophenyl | 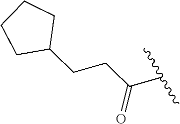 [SMILES: *C(=O)CCC1CCCC1] |
| 4-chlorophenyl | 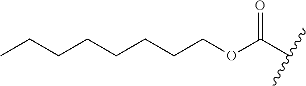 [SMILES: *C(=O)OCCCCCCCC] |
| 4-chlorophenyl | 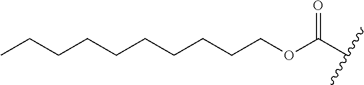 [SMILES: *C(=O)OCCCCCCCCCC] |
| 4-chlorophenyl | 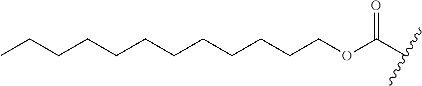 [SMILES: *C(=O)OCCCCCCCCCCCC] |
| 4-chlorophenyl | 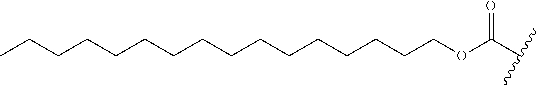 [SMILES: *C(=O)OCCCCCCCCCCCCCCCC] |
| 4-chlorophenyl | 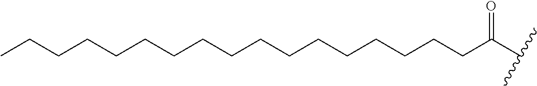 [SMILES: *C(=O)CCCCCCCCCCCCCCCCC] |
| 4-chlorophenyl | 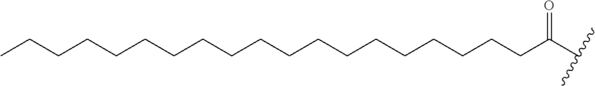 [SMILES: *C(=O)CCCCCCCCCCCCCCCCCCC] |
| 4-chlorophenyl | 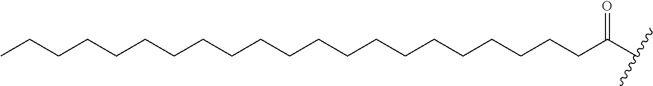 [SMILES: *C(=O)CCCCCCCCCCCCCCCCCCCCC] |
| 4-chlorophenyl | 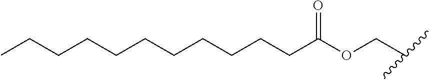 [SMILES: *COC(=O)CCCCCCCCCCC] |
| 4-chlorophenyl | 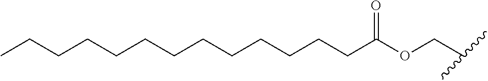 [SMILES: *COC(=O)CCCCCCCCCCCCC] |
| 4-chlroophenyl | 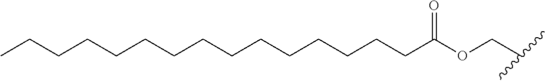 [SMILES: *COC(=O)CCCCCCCCCCCCCCC] |
| 4-chlorophenyl | 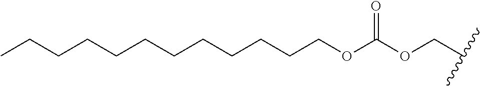 [SMILES: *COC(=O)OCCCCCCCCCCCC] |
| 4-chlorophenyl | 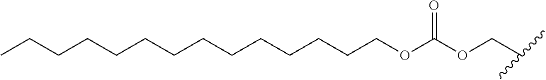 [SMILES: *COC(=O)OCCCCCCCCCCCCCC] |
| 4-chlorophenyl | 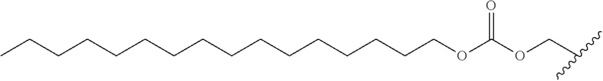 [SMILES: *COC(=O)OCCCCCCCCCCCCCCCC] |
| 3,4-dichlorophenyl | 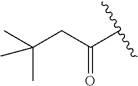 [SMILES: *C(=O)CC(C)(C)C] |
| 3,4-dichlorophenyl | 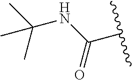 [SMILES: *C(=O)NC(C)(C)C] |
| 3,4-dichlorophenyl | 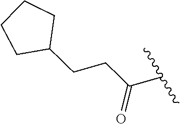 [SMILES: *C(=O)CCC1CCCC1] |
| 3,4-dichlorophenyl | 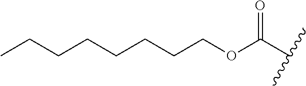 [SMILES: *C(=O)OCCCCCCCC] |
| 3,4-dichlorophenyl | 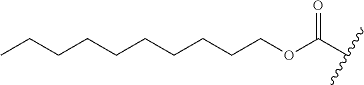 [SMILES: *C(=O)OCCCCCCCCCC] |
| 3,4-dichlorophenyl | 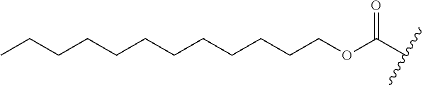 [SMILES: *C(=O)OCCCCCCCCCCCC] |
| 3,4-dichlorophenyl | 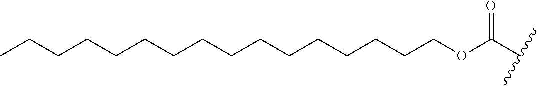 [SMILES: *C(=O)OCCCCCCCCCCCCCCCC] |
| 3,4-dichlorophenyl | 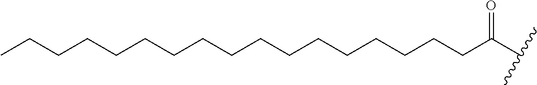 [SMILES: *C(=O)CCCCCCCCCCCCCCCCC] |
| 3,4-dichlorophenyl | 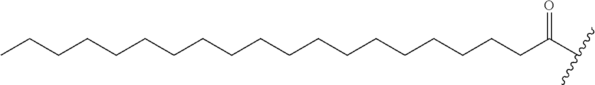 [SMILES: *C(=O)CCCCCCCCCCCCCCCCCCC] |
| 3,4-dichlorophenyl | 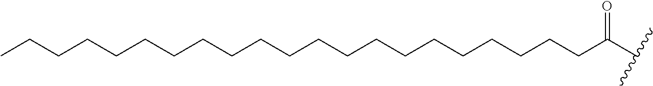 [SMILES: *C(=O)CCCCCCCCCCCCCCCCCCCCC] |
| 3,4-dichlorophenyl | 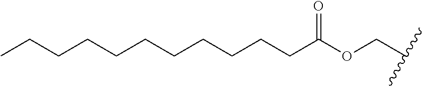 [SMILES: *COC(=O)CCCCCCCCCCC] |
| 3,4-dichlorophenyl | 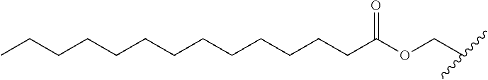 [SMILES: *COC(=O)CCCCCCCCCCCCC] |
| 3,4-dichlorophenyl | 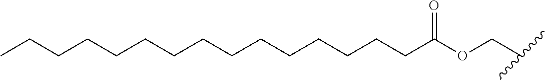 [SMILES: *COC(=O)CCCCCCCCCCCCCCC] |
| 3,4-dichlorophenyl | 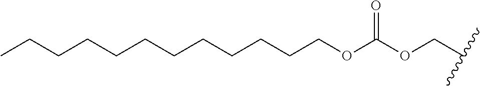 [SMILES: *COC(=O)OCCCCCCCCCCCC] |
| 3,4-dichlorophenyl | 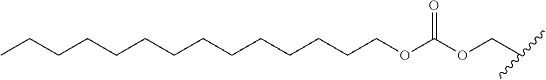 [SMILES: *COC(=O)OCCCCCCCCCCCCCC] |
| 3,4-dichlorophenyl | 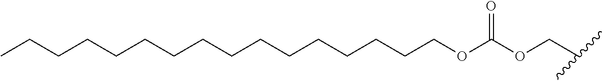 [SMILES: *COC(=O)OCCCCCCCCCCCCCCCC] |
| 4-methylphenyl | 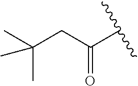 [SMILES: *C(=O)CC(C)(C)C] |
| 4-methylphenyl | 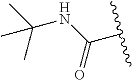 [SMILES: *C(=O)NC(C)(C)C] |
| 4-methylphenyl | 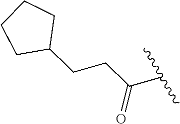 [SMILES: *C(=O)CCC1CCCC1] |
| 4-methylphenyl | 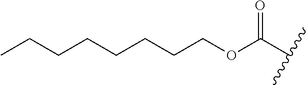 [SMILES: *C(=O)OCCCCCCCC] |
| 4-methylphenyl | 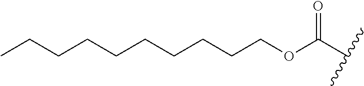 [SMILES: *C(=O)OCCCCCCCCCC] |
| 4-methylphenyl | 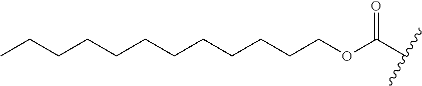 [SMILES: *C(=O)OCCCCCCCCCCCC] |
| 4-methylphenyl | 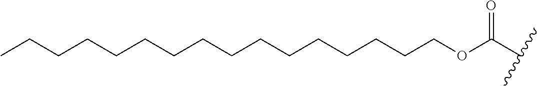 [SMILES: *C(=O)OCCCCCCCCCCCCCCCC] |
| 4-methylphenyl | 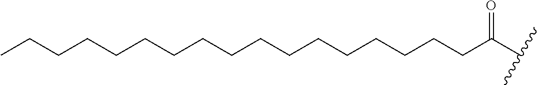 [SMILES: *C(=O)CCCCCCCCCCCCCCCCC] |
| 4-methylphenyl | 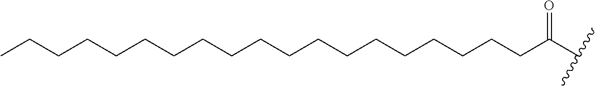 [SMILES: *C(=O)CCCCCCCCCCCCCCCCCCC] |
| 4-methylphenyl | 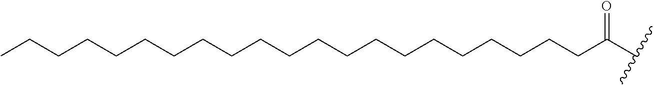 [SMILES: *C(=O)CCCCCCCCCCCCCCCCCCCCC] |
| 4-methylphenyl | 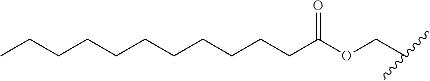 [SMILES: *COC(=O)CCCCCCCCCCC] |
| 4-methylphenyl | 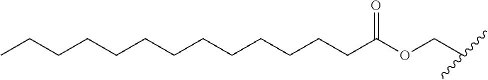 [SMILES: *COC(=O)CCCCCCCCCCCCC] |
| 4-methylphenyl | 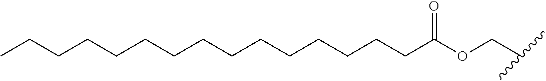 [SMILES: *COC(=O)CCCCCCCCCCCCCCC] |
| 4-methylphenyl | 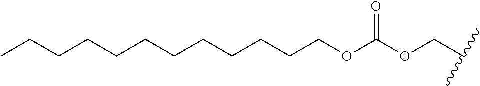 [SMILES: *COC(=O)OCCCCCCCCCCCC] |
| 4-methylphenyl | 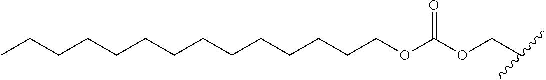 [SMILES: *COC(=O)OCCCCCCCCCCCCCC] |
| 4-methylphenyl | 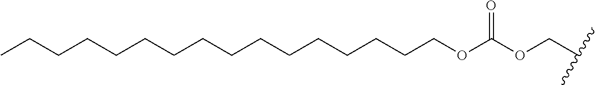 [SMILES: *COC(=O)OCCCCCCCCCCCCCCCC] |
| 4-methoxyphenyl | 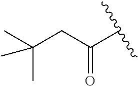 [SMILES: *C(=O)CC(C)(C)C] |
| 4-methoxyphenyl | 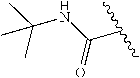 [SMILES: *C(=O)NC(C)(C)C] |
| 4-methoxyphenyl | 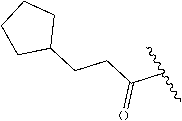 [SMILES: *C(=O)CCC1CCCC1] |
| 4-methoxyphenyl | 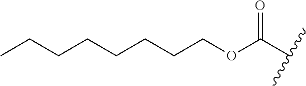 [SMILES: *C(=O)OCCCCCCCC] |
| 4-methoxyphenyl | 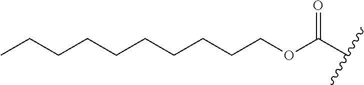 [SMILES: *C(=O)OCCCCCCCCCC] |
| 4-methoxyphenyl | 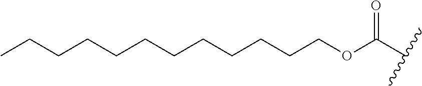 [SMILES: *C(=O)OCCCCCCCCCCCC] |
| 4-methoxyphenyl | 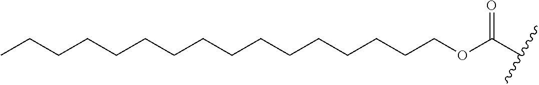 [SMILES: *C(=O)OCCCCCCCCCCCCCCCC] |
| 4-methoxyphenyl | 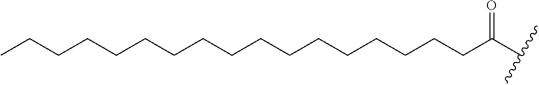 [SMILES: *C(=O)CCCCCCCCCCCCCCCCC] |
| 4-methoxyphenyl | 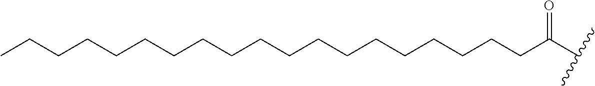 [SMILES: *C(=O)CCCCCCCCCCCCCCCCCCC] |
| 4-methoxyphenyl | 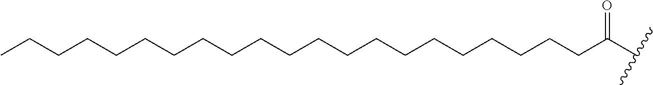 [SMILES: *C(=O)CCCCCCCCCCCCCCCCCCCCC] |
| 4-methoxyphenyl | 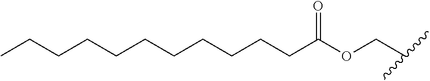 [SMILES: *COC(=O)CCCCCCCCCCC] |
| 4-methoxyphenyl | 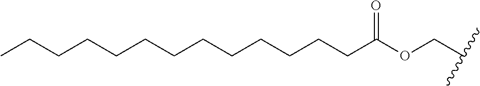 [SMILES: *COC(=O)CCCCCCCCCCCCC] |
| 4-methoxyphenyl | 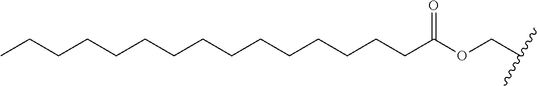 [SMILES: *COC(=O)CCCCCCCCCCCCCCC] |
| 4-methoxyphenyl | 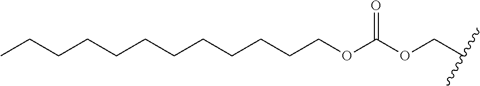 [SMILES: *COC(=O)OCCCCCCCCCCCC] |
| 4-methoxyphenyl | 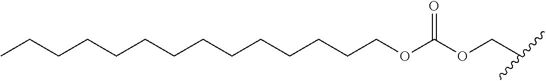 [SMILES: *COC(=O)OCCCCCCCCCCCCCC] |
| 4-methoxyphenyl | 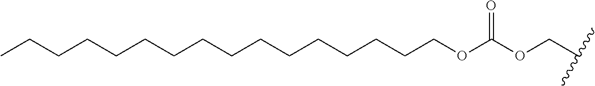 [SMILES: *COC(=O)OCCCCCCCCCCCCCCCC] |

In a 64^{th} aspect provided herein is a compound, or pharmaceutically acceptable salt thereof, having a structure provided in Formula (I-21),

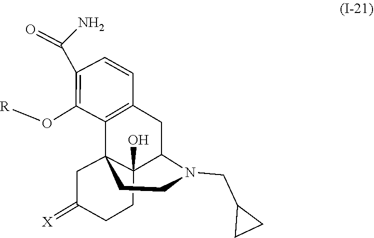

wherein X is ═O, R is R⁴OP(O)[OCH₂—OC(O)CH(CH₃)₂]—O—CH(R³)— and R³ and R⁴ are any of the pair of R³/R⁴ groups recited in Table 11. Alternatively, X is ═CH₂, R is
R⁴OP(O)[OCH₂—OC(O)CH(CH₃)₂]—O—CH(R³)— and R³ and R⁴ are any of the pair of R³/R⁴ groups recited in Table 11.

In a 65^{th} aspect, provided herein is a compound, or pharmaceutically acceptable salt thereof, having a structure provided in Formula (II-21),

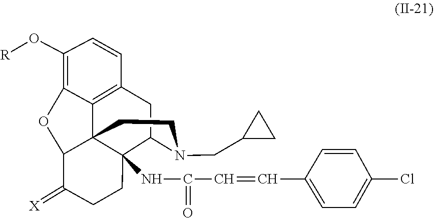

wherein X is ═O, R is R⁴OP(O)[OCH₂—OC(O)CH(CH₃)₂]—O—CH(R³)— and R³ and R⁴ are any of the pair of R³/R⁴ groups recited in Table 11. Alternatively, X is ═CH₂R is
R⁴OP(O)[OCH₂—OC(O)CH(CH₃)₂]—O—CH(R³)— and R³ and R⁴ are any of the pair of R³/R⁴ groups recited in Table 11.

In a 66^{th} aspect, provided herein is a compound, or pharmaceutically acceptable salt thereof, having a structure provided in Formula (III-21),

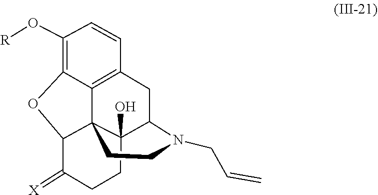

wherein X is ═O, R is R⁴OP(O)[OCH₂—OC(O)CH(CH₃)₂]—O—CH(R³)— and R³ and R⁴ are any of the pair of R³/R⁴ groups recited in Table 11. Alternatively, X is ═CH₂R is
R⁴OP(O)[OCH₂—OC(O)CH(CH₃)₂]—O—CH(R³)— and R³ and R⁴ are any of the pair of R³/R⁴ groups recited in Table 11.

TABLE 11

| R³ | R⁴ |
|---|---|
| H | Me |
| H | —C₇H₁₅ |
| H | —C₉H₁₉ |
| H | —C₁₁H₂₃ |
| H | —C₁₃H₂₇ |
| H | —C₁₅H₃₁ |
| H | —C₁₇H₃₅ |
| H | 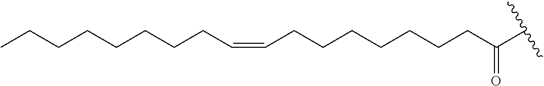 [SMILES: *C(=O)CCCCCCC/C=C\CCCCCCCC] |
| H | 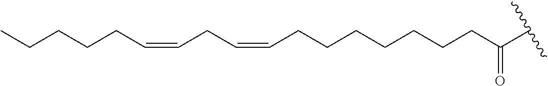 [SMILES: *C(=O)CCCCCCC/C=C\C/C=C\CCCCC] |
| H | 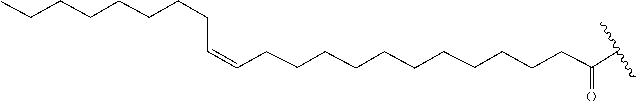 [SMILES: *C(=O)CCCCCCCCCCC/C=C\CCCCCCCC] |
| H | 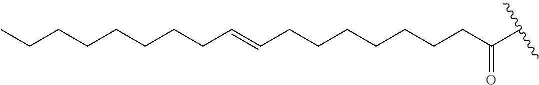 [SMILES: *C(=O)CCCCCCC/C=C/CCCCCCCC] |
| H | 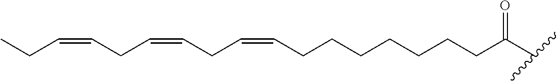 [SMILES: *C(=O)CCCCCCC/C=C\C/C=C\C/C=C\CC] |
| H | tBu |
| H | phenyl |
| H | 4-chlorophenyl |
| H | 3,4-dichlorophenyl |
| H | 4-methylphenyl |
| H | 4-methoxyphenyl |
| Me | Me |
| Me | —C₇H₁₅ |
| Me | —C₉H₁₉ |
| Me | —C₁₁H₂₃ |
| Me | —C₁₃H₂₇ |
| Me | —C₁₅H₃₁ |
| Me | —C₁₇H₃₅ |
| Me | 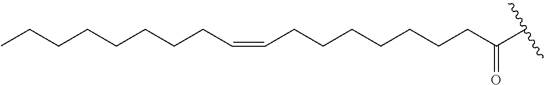 [SMILES: *C(=O)CCCCCCC/C=C\CCCCCCCC] |
| Me | 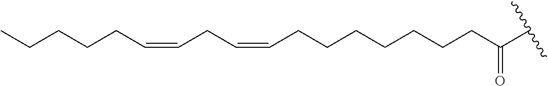 [SMILES: *C(=O)CCCCCCC/C=C\C/C=C\CCCCC] |
| Me | 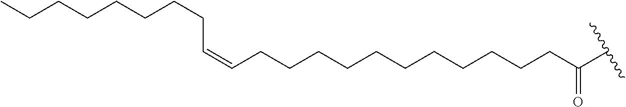 [SMILES: *C(=O)CCCCCCCCCCC/C=C\CCCCCCCC] |
| Me | 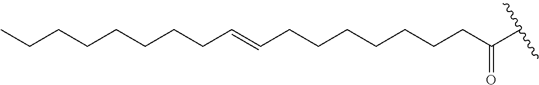 [SMILES: *C(=O)CCCCCCC/C=C/CCCCCCCC] |
| Me | 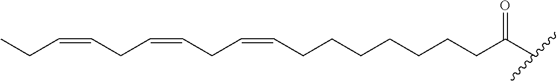 [SMILES: *C(=O)CCCCCCC/C=C\C/C=C\C/C=C\CC] |
| Me | tBu |
| Me | phenyl |
| Me | 4-chlorophenyl |
| Me | 3,4-dichlorophenyl |
| Me | 4-methylphenyl |
| Me | 4-methoxyphenyl |
| tBu | Me |
| tBu | —C₇H₁₅ |
| tBu | —C₉H₁₉ |
| tBu | —C₁₁H₂₃ |
| tBu | —C₁₃H₂₇ |
| tBu | —C₁₅H₃₁ |
| tBu | —C₁₇H₃₅ |
| tBu | 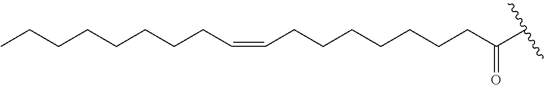 [SMILES: *C(=O)CCCCCCC/C=C\CCCCCCCC] |
| tBu | 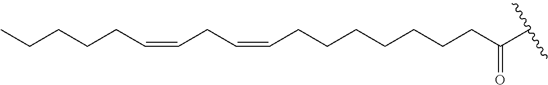 [SMILES: *C(=O)CCCCCCC/C=C\C/C=C\CCCCC] |
| tBu | 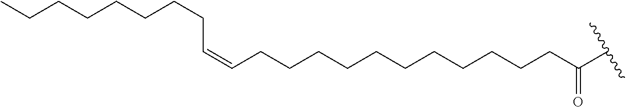 [SMILES: *C(=O)CCCCCCCCCCC/C=C\CCCCCCCC] |
| tBu | 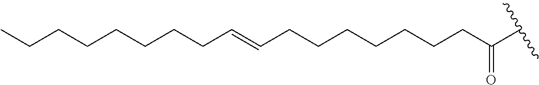 [SMILES: *C(=O)CCCCCCC/C=C/CCCCCCCC] |
| tBu | 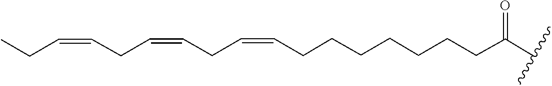 [SMILES: *C(=O)CCCCCCC/C=C\C/C=C\C/C=C\CC] |
| tBu | phenyl |
| tBu | tBu |
| tBu | 4-chlorophenyl |
| tBu | 3,4-dichlorophenyl |
| tBu | 4-methylphenyl |
| tBu | 4-methoxyphenyl |
| —C₇H₁₅ | Me |
| —C₇H₁₅ | —C₇H₁₅ |
| —C₇H₁₅ | —C₉H₁₉ |
| —C₇H₁₅ | —C₁₁H₂₃ |
| —C₇H₁₅ | —C₁₃H₂₇ |
| —C₇H₁₅ | —C₁₅H₃₁ |
| —C₇H₁₅ | —C₁₇H₃₅ |
| —C₇H₁₅ | 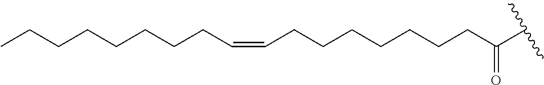 [SMILES: *C(=O)CCCCCCC/C=C\CCCCCCCC] |
| —C₇H₁₅ | 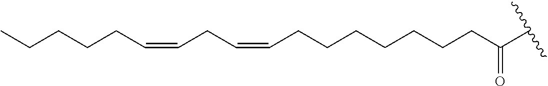 [SMILES: *C(=O)CCCCCCC/C=C\C/C=C\CCCCC] |
| —C₇H₁₅ | 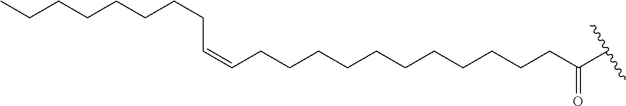 [SMILES: *C(=O)CCCCCCCCCCC/C=C\CCCCCCCC] |
| —C₇H₁₅ | 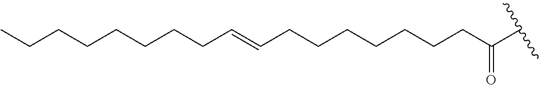 [SMILES: *C(=O)CCCCCCC/C=C/CCCCCCCC] |
| —C₇H₁₅ | 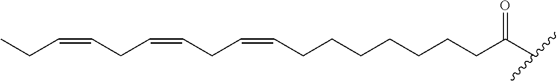 [SMILES: *C(=O)CCCCCCC/C=C\C/C=C\C/C=C\CC] |
| —C₇H₁₅ | tBu |
| —C₇H₁₅ | phenyl |
| —C₇H₁₅ | 4-chlorophenyl |
| —C₇H₁₅ | 3,4-dichlorophenyl |
| —C₇H₁₅ | 4-methylphenyl |
| —C₇H₁₅ | 4-methoxyphenyl |
| —C₉H₁₉ | Me |
| —C₉H₁₉ | —C₇H₁₅ |
| —C₉H₁₉ | —C₉H₁₉ |
| —C₉H₁₉ | —C₁₁H₂₃ |
| —C₉H₁₉ | —C₁₃H₂₇ |
| —C₉H₁₉ | —C₁₅H₃₁ |
| —C₉H₁₉ | —C₁₇H₃₅ |
| —C₉H₁₉ | 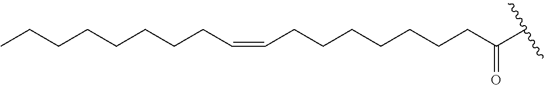 [SMILES: *C(=O)CCCCCCC/C=C\CCCCCCCC] |
| —C₉H₁₉ | 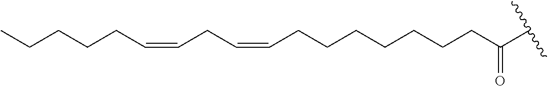 [SMILES: *C(=O)CCCCCCC/C=C\C/C=C\CCCCC] |
| —C₉H₁₉ | 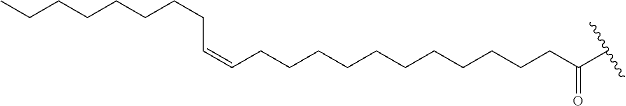 [SMILES: *C(=O)CCCCCCCCCCC/C=C\CCCCCCCC] |
| —C₉H₁₉ | 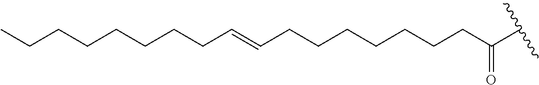 [SMILES: *C(=O)CCCCCCC/C=C/CCCCCCCC] |
| —C₉H₁₉ | 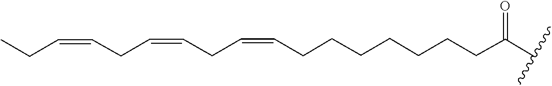 [SMILES: *C(=O)CCCCCCC/C=C\C/C=C\C/C=C\CC] |
| —C₉H₁₉ | tBu |
| —C₉H₁₉ | phenyl |
| —C₉H₁₉ | 4-chlorophenyl |
| —C₉H₁₉ | 3,4-dichlorophenyl |
| —C₉H₁₉ | 4-methylphenyl |
| —C₉H₁₉ | 4-methoxyphenyl |
| —C₁₁H₂₃ | Me |
| —C₁₁H₂₃ | —C₇H₁₅ |
| —C₁₁H₂₃ | —C₉H₁₉ |
| —C₁₁H₂₃ | —C₁₁H₂₃ |
| —C₁₁H₂₃ | —C₁₃H₂₇ |
| —C₁₁H₂₃ | —C₁₅H₃₁ |
| —C₁₁H₂₃ | —C₁₇H₃₅ |
| —C₁₁H₂₃ | 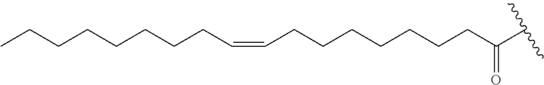 [SMILES: *C(=O)CCCCCCC/C=C\CCCCCCCC] |
| —C₁₁H₂₃ | 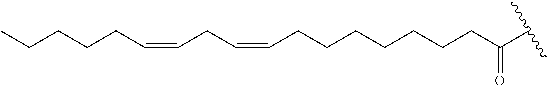 [SMILES: *C(=O)CCCCCCC/C=C\C/C=C\CCCCC] |
| —C₁₁H₂₃ | 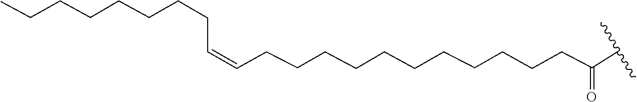 [SMILES: *C(=O)CCCCCCCCCCC/C=C\CCCCCCCC] |
| —C₁₁H₂₃ | 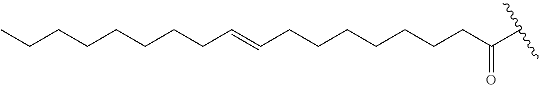 [SMILES: *C(=O)CCCCCCC/C=C/CCCCCCCC] |
| —C₁₁H₂₃ | 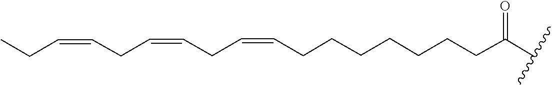 [SMILES: *C(=O)CCCCCCC/C=C\C/C=C\C/C=C\CC] |
| —C₁₁H₂₃ | tBu |
| —C₁₁H₂₃ | phenyl |
| —C₁₁H₂₃ | 4-chlorophenyl |
| —C₁₁H₂₃ | 3,4-dichlorophenyl |
| —C₁₁H₂₃ | 4-methylphenyl |
| —C₁₁H₂₃ | 4-methoxyphenyl |
| —C₁₃H₂₇ | Me |
| —C₁₃H₂₇ | —C₇H₁₅ |
| —C₁₃H₂₇ | —C₉H₁₉ |
| —C₁₃H₂₇ | —C₁₁H₂₃ |
| —C₁₃H₂₇ | —C₁₃H₂₇ |
| —C₁₃H₂₇ | —C₁₅H₃₁ |
| —C₁₃H₂₇ | —C₁₇H₃₅ |
| —C₁₃H₂₇ | 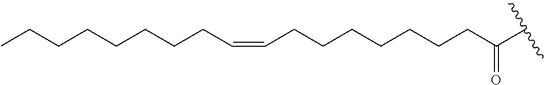 [SMILES: *C(=O)CCCCCCC/C=C\CCCCCCCC] |
| —C₁₃H₂₇ | 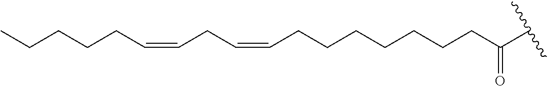 [SMILES: *C(=O)CCCCCCC/C=C\C/C=C\CCCCC] |
| —C₁₃H₂₇ | 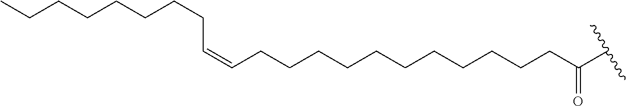 [SMILES: *C(=O)CCCCCCCCCCC/C=C\CCCCCCCC] |
| —C₁₃H₂₇ | 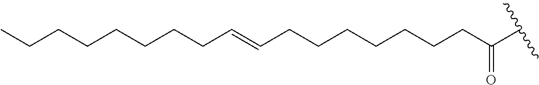 [SMILES: *C(=O)CCCCCCC/C=C/CCCCCCCC] |
| —C₁₃H₂₇ | 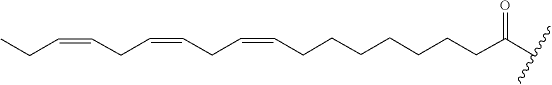 [SMILES: *C(=O)CCCCCCC/C=C\C/C=C\C/C=C\CC] |
| —C₁₃H₂₇ | tBu |
| —C₁₃H₂₇ | phenyl |
| —C₁₃H₂₇ | 4-chlorophenyl |
| —C₁₃H₂₇ | 3,4-dichlorophenyl |
| —C₁₃H₂₇ | 4-methylphenyl |
| —C₁₃H₂₇ | 4-methoxyphenyl |
| —C₁₅H₃₁ | Me |
| —C₁₅H₃₁ | —C₇H₁₅ |
| —C₁₅H₃₁ | —C₉H₁₉ |
| —C₁₅H₃₁ | —C₁₁H₂₃ |
| —C₁₅H₃₁ | —C₁₃H₂₇ |
| —C₁₅H₃₁ | —C₁₅H₃₁ |
| —C₁₅H₃₁ | —C₁₇H₃₅ |
| —C₁₅H₃₁ | 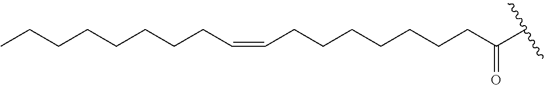 [SMILES: *C(=O)CCCCCCC/C=C\CCCCCCCC] |
| —C₁₅H₃₁ | 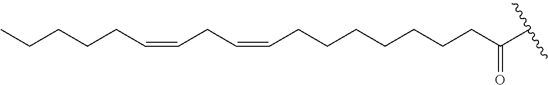 [SMILES: *C(=O)CCCCCCC/C=C\C/C=C\CCCCC] |
| —C₁₅H₃₁ | 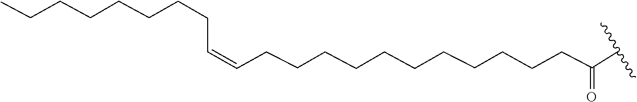 [SMILES: *C(=O)CCCCCCCCCCC/C=C\CCCCCCCC] |
| —C₁₅H₃₁ | 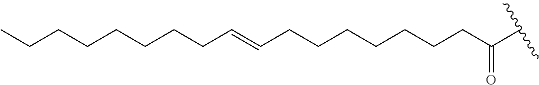 [SMILES: *C(=O)CCCCCCC/C=C/CCCCCCCC] |
| —C₁₅H₃₁ | 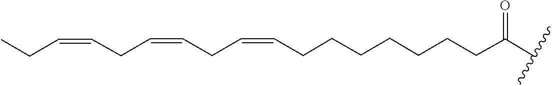 [SMILES: *C(=O)CCCCCCC/C=C\C/C=C\C/C=C\CC] |
| —C₁₅H₃₁ | tBu |
| —C₁₅H₃₁ | phenyl |
| —C₁₅H₃₁ | 4-chlorophenyl |
| —C₁₅H₃₁ | 3,4-dichlorophenyl |
| —C₁₅H₃₁ | 4-methylphenyl |
| —C₁₅H₃₁ | 4-methoxyphenyl |
| —C₁₇H₃₅ | Me |
| —C₁₇H₃₅ | —C₇H₁₅ |
| —C₁₇H₃₅ | —C₉H₁₉ |
| —C₁₇H₃₅ | —C₁₁H₂₃ |
| —C₁₇H₃₅ | —C₁₃H₂₇ |
| —C₁₇H₃₅ | —C₁₅H₃₁ |
| —C₁₇H₃₅ | —C₁₇H₃₅ |
| —C₁₇H₃₅ | 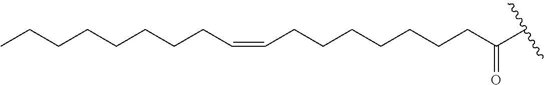 [SMILES: *C(=O)CCCCCCC/C=C\CCCCCCCC] |
| —C₁₇H₃₅ | 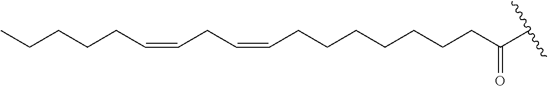 [SMILES: *C(=O)CCCCCCC/C=C\C/C=C\CCCCC] |
| —C₁₇H₃₅ | 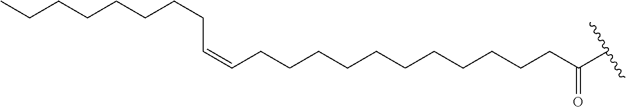 [SMILES: *C(=O)CCCCCCCCCCC/C=C\CCCCCCCC] |
| —C₁₇H₃₅ | 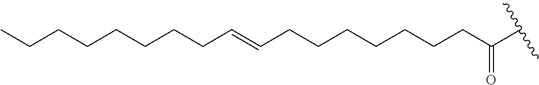 [SMILES: *C(=O)CCCCCCC/C=C/CCCCCCCC] |
| —C₁₇H₃₅ | 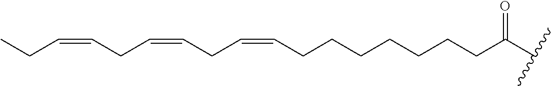 [SMILES: *C(=O)CCCCCCC/C=C\C/C=C\C/C=C\CC] |
| —C₁₇H₃₅ | tBu |
| —C₁₇H₃₅ | phenyl |
| —C₁₇H₃₅ | 4-chlorophenyl |
| —C₁₇H₃₅ | 3,4-dichlorophenyl |
| —C₁₇H₃₅ | 4-methylphenyl |
| —C₁₇H₃₅ | 4-methoxyphenyl |
| Phenyl | Me |
| Phenyl | —C₇H₁₅ |
| Phenyl | —C₉H₁₉ |
| Phenyl | —C₁₁H₂₃ |
| Phenyl | —C₁₃H₂₇ |
| Phenyl | —C₁₅H₃₁ |
| Phenyl | —C₁₇H₃₅ |
| Phenyl | 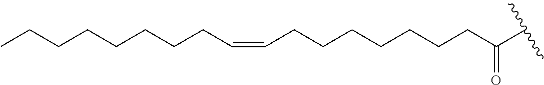 [SMILES: *C(=O)CCCCCCC/C=C\CCCCCCCC] |
| Phenyl | 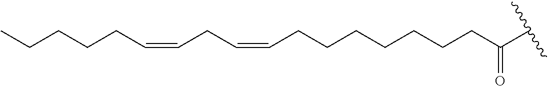 [SMILES: *C(=O)CCCCCCC/C=C\C/C=C\CCCCC] |
| Phenyl | 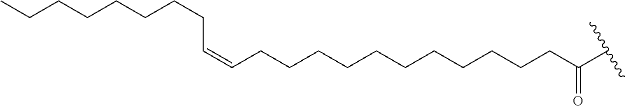 [SMILES: *C(=O)CCCCCCCCCCC/C=C\CCCCCCCC] |
| Phenyl | 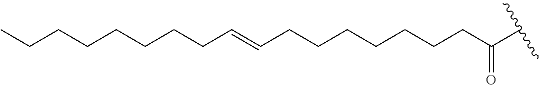 [SMILES: *C(=O)CCCCCCC/C=C/CCCCCCCC] |
| Phenyl | 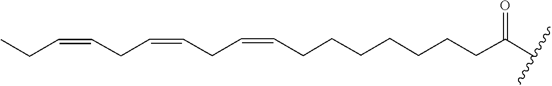 [SMILES: *C(=O)CCCCCCC/C=C\C/C=C\C/C=C\CC] |
| Phenyl | tBu |
| Phenyl | phenyl |
| Phenyl | 4-chlorophenyl |
| Phenyl | 3,4-dichlorophenyl |
| Phenyl | 4-methylphenyl |
| Phenyl | 4-methoxyphenyl |
| 4-chlorophenyl | Me |
| 4-chlorophenyl | —C₇H₁₅ |
| 4-chlorophenyl | —C₉H₁₉ |
| 4-chlorophenyl | —C₁₁H₂₃ |
| 4-chlorophenyl | —C₁₃H₂₇ |
| 4-chlorophenyl | —C₁₅H₃₁ |
| 4-chlorophenyl | —C₁₇H₃₅ |
| 4-chlorophenyl | 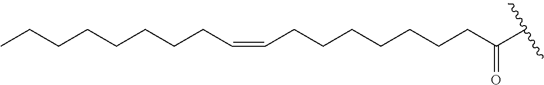 [SMILES: *C(=O)CCCCCCC/C=C\CCCCCCCC] |
| 4-chlorophenyl | 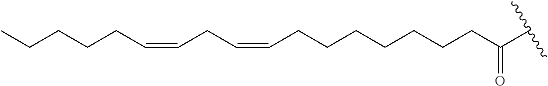 [SMILES: *C(=O)CCCCCCC/C=C\C/C=C\CCCCC] |
| 4-chlorophenyl | 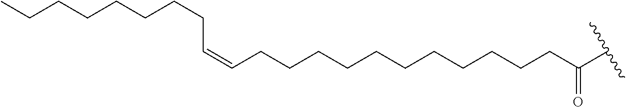 [SMILES: *C(=O)CCCCCCCCCCC/C=C\CCCCCCCC] |
| 4-chlorophenyl | 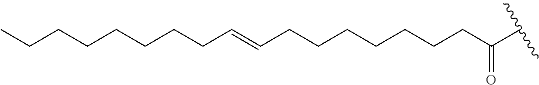 [SMILES: *C(=O)CCCCCCC/C=C/CCCCCCCC] |
| 4-chlorophenyl | 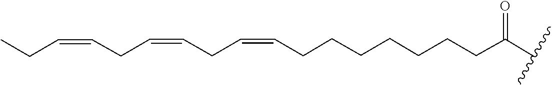 [SMILES: *C(=O)CCCCCCC/C=C\C/C=C\C/C=C\CC] |
| 4-chlorophenyl | tBu |
| 4-chlorophenyl | phenyl |
| 4-chlorophenyl | 4-chlorophenyl |
| 4-chlorophenyl | 3,4-dichlorophenyl |
| 4-chlorophenyl | 4-methylphenyl |
| 4-chlorophenyl | 4-methoxyphenyl |
| 3,4-dichlorophenyl | Me |
| 3,4-dichlorophenyl | —C₇H₁₅ |
| 3,4-dichlorophenyl | —C₉H₁₉ |
| 3,4-dichlorophenyl | —C₁₁H₂₃ |
| 3,4-dichlorophenyl | —C₁₃H₂₇ |
| 3,4-dichlorophenyl | —C₁₅H₃₁ |
| 3,4-dichlorophenyl | —C₁₇H₃₅ |
| 3,4-dichlorophenyl | 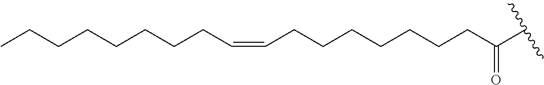 [SMILES: *C(=O)CCCCCCC/C=C\CCCCCCCC] |
| 3,4-dichlorophenyl | 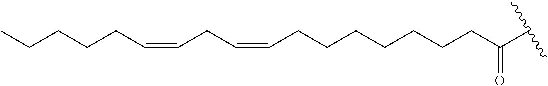 [SMILES: *C(=O)CCCCCCC/C=C\C/C=C\CCCCC] |
| 3,4-dichlorophenyl | 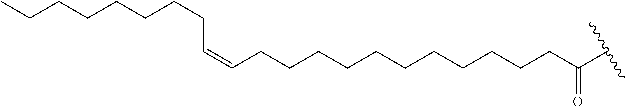 [SMILES: *C(=O)CCCCCCCCCCC/C=C\CCCCCCCC] |
| 3,4-dichlorophenyl | 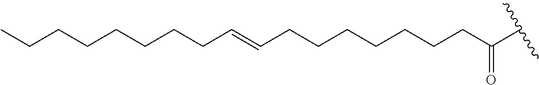 [SMILES: *C(=O)CCCCCCC/C=C/CCCCCCCC] |
| 3,4-dichlorophenyl | 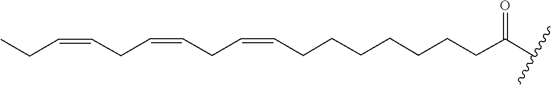 [SMILES: *C(=O)CCCCCCC/C=C\C/C=C\C/C=C\CC] |
| 3,4-dichlorophenyl | tBu |
| 3,4-dichlorophenyl | phenyl |
| 3,4-dichlorophenyl | 4-chlorophenyl |
| 3,4-dichlorophenyl | 3,4-dichlorophenyl |
| 3,4-dichlorophenyl | 4-methylphenyl |
| 3,4-dichlorophenyl | 4-methoxyphenyl |
| 4-methylphenyl | Me |
| 4-methylphenyl | —C₇H₁₅ |
| 4-methylphenyl | —C₉H₁₉ |
| 4-methylphenyl | —C₁₁H₂₃ |
| 4-methylphenyl | —C₁₃H₂₇ |
| 4-methylphenyl | —C₁₅H₃₁ |
| 4-methylphenyl | —C₁₇H₃₅ |
| 4-methylphenyl | 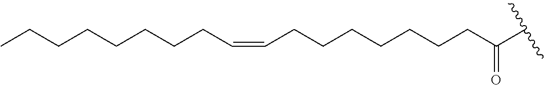 [SMILES: *C(=O)CCCCCCC/C=C\CCCCCCCC] |
| 4-methylphenyl | 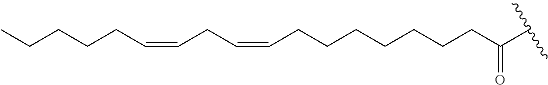 [SMILES: *C(=O)CCCCCCC/C=C\C/C=C\CCCCC] |
| 4-methylphenyl | 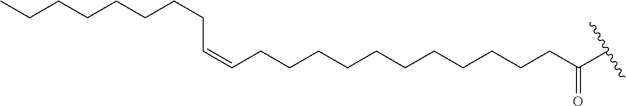 [SMILES: *C(=O)CCCCCCCCCCC/C=C\CCCCCCCC] |
| 4-methylphenyl | 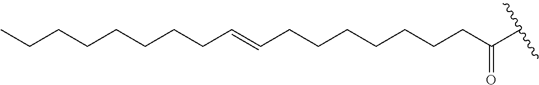 [SMILES: *C(=O)CCCCCCC/C=C/CCCCCCCC] |
| 4-methylphenyl | 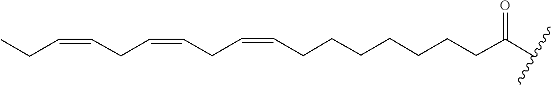 [SMILES: *C(=O)CCCCCCC/C=C\C/C=C\C/C=C\CC] |
| 4-methylphenyl | tBu |
| 4-methylphenyl | phenyl |
| 4-methylphenyl | 4-chlorophenyl |
| 4-methylphenyl | 3,4-dichlorophenyl |
| 4-methylphenyl | 4-methylphenyl |
| 4-methylphenyl | 4-methoxyphenyl |
| 4-methoxyphenyl | Me |
| 4-methoxyphenyl | —C₇H₁₅ |
| 4-methoxyphenyl | —C₉H₁₉ |
| 4-methoxyphenyl | —C₁₁H₂₃ |
| 4-methoxyphenyl | —C₁₃H₂₇ |
| 4-methoxyphenyl | —C₁₅H₃₁ |
| 4-methoxyphenyl | —C₁₇H₃₅ |
| 4-methoxyphenyl | 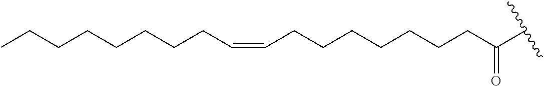 [SMILES: *C(=O)CCCCCCC/C=C\CCCCCCCC] |
| 4-methoxyphenyl | 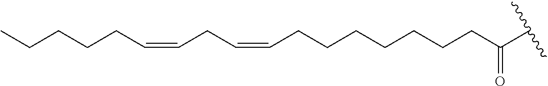 [SMILES: *C(=O)CCCCCCC/C=C\C/C=C\CCCCC] |
| 4-methoxyphenyl | 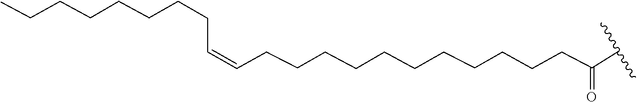 [SMILES: *C(=O)CCCCCCCCCCC/C=C\CCCCCCCC] |
| 4-methoxyphenyl | 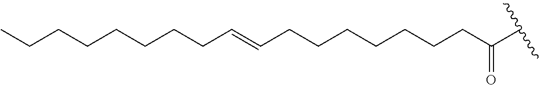 [SMILES: *C(=O)CCCCCCC/C=C/CCCCCCCC] |
| 4-methoxyphenyl | 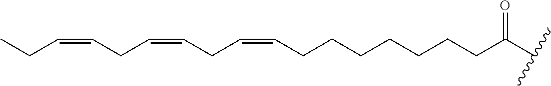 [SMILES: *C(=O)CCCCCCC/C=C\C/C=C\C/C=C\CC] |
| 4-methoxyphenyl | tBu |
| 4-methoxyphenyl | phenyl |
| 4-methoxyphenyl | 4-chlorophenyl |
| 4-methoxyphenyl | 3,4-dichlorophenyl |
| 4-methoxyphenyl | 4-methylphenyl |
| 4-methoxyphenyl | 4-methoxyphenyl |
| 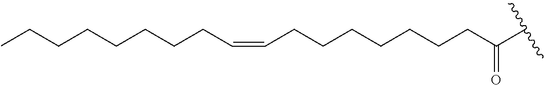 [SMILES: *C(=O)CCCCCCC/C=C\CCCCCCCC] | Me |
| 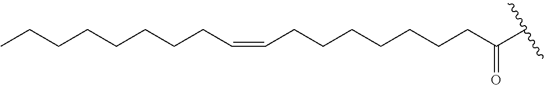 [SMILES: *C(=O)CCCCCCC/C=C\CCCCCCCC] | —C₇H₁₅ |
| 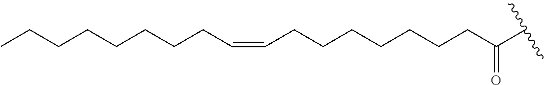 [SMILES: *C(=O)CCCCCCC/C=C\CCCCCCCC] | —C₉H₁₉ |
| 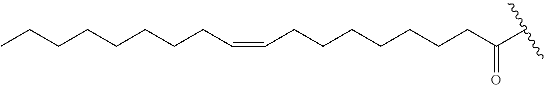 [SMILES: *C(=O)CCCCCCC/C=C\CCCCCCCC] | —C₁₁H₂₃ |
| 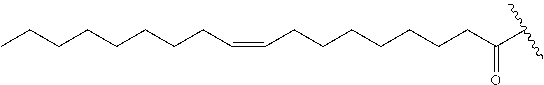 [SMILES: *C(=O)CCCCCCC/C=C\CCCCCCCC] | —C₁₃H₂₇ |
| 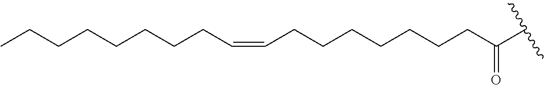 [SMILES: *C(=O)CCCCCCC/C=C\CCCCCCCC] | —C₁₅H₃₁ |
| 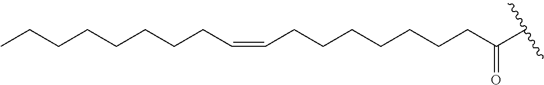 [SMILES: *C(=O)CCCCCCC/C=C\CCCCCCCC] | —C₁₇H₃₅ |
| 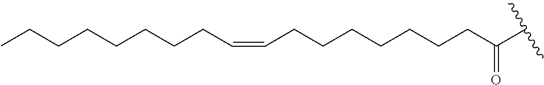 [SMILES: *C(=O)CCCCCCC/C=C\CCCCCCCC] | 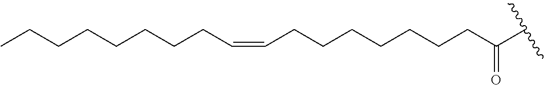 [SMILES: *C(=O)CCCCCCC/C=C\CCCCCCCC] |
| 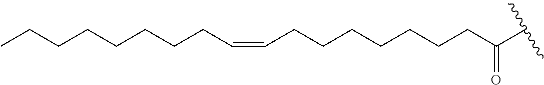 [SMILES: *C(=O)CCCCCCC/C=C\CCCCCCCC] | 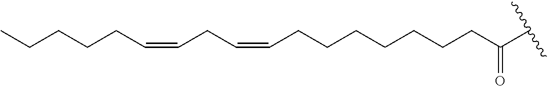 [SMILES: *C(=O)CCCCCCC/C=C\C/C=C\CCCCC] |
| 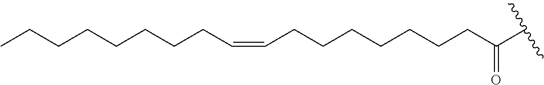 [SMILES: *C(=O)CCCCCCC/C=C\CCCCCCCC] | 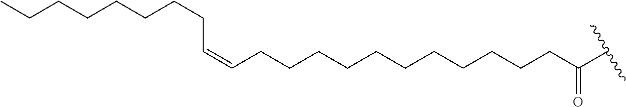 [SMILES: *C(=O)CCCCCCCCCCC/C=C\CCCCCCCC] |
| 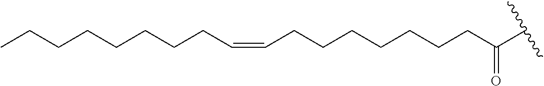 [SMILES: *C(=O)CCCCCCC/C=C\CCCCCCCC] | 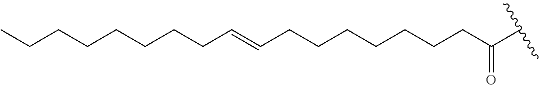 [SMILES: *C(=O)CCCCCCC/C=C/CCCCCCCC] |
| 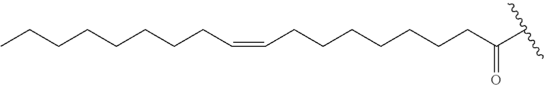 [SMILES: *C(=O)CCCCCCC/C=C\CCCCCCCC] | 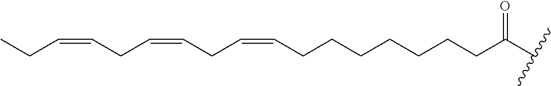 [SMILES: *C(=O)CCCCCCC/C=C\C/C=C\C/C=C\CC] |
| 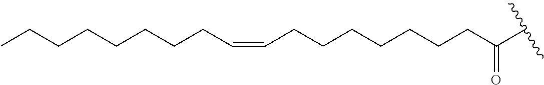 [SMILES: *C(=O)CCCCCCC/C=C\CCCCCCCC] | tBu |
| 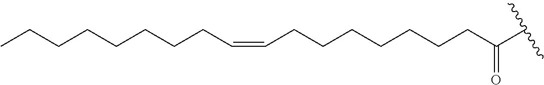 [SMILES: *C(=O)CCCCCCC/C=C\CCCCCCCC] | phenyl |
| 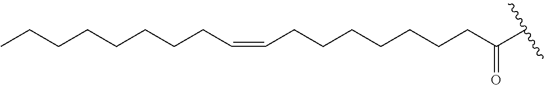 [SMILES: *C(=O)CCCCCCC/C=C\CCCCCCCC] | 4-chlorophenyl |
| 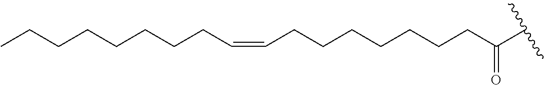 [SMILES: *C(=O)CCCCCCC/C=C\CCCCCCCC] | 3,4-dichlorophenyl |
| 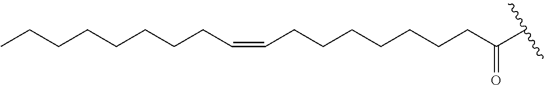 [SMILES: *C(=O)CCCCCCC/C=C\CCCCCCCC] | 4-methylphenyl |
| 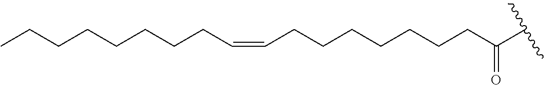 [SMILES: *C(=O)CCCCCCC/C=C\CCCCCCCC] | 4-methoxyphenyl |
| 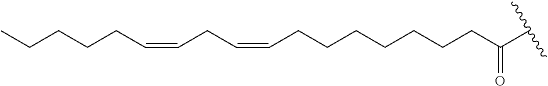 [SMILES: *C(=O)CCCCCCC/C=C\C/C=C\CCCCC] | Me |
| 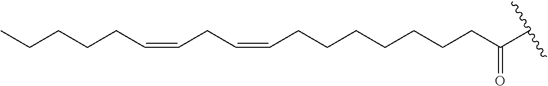 [SMILES: *C(=O)CCCCCCC/C=C\C/C=C\CCCCC] | —C₇H₁₅ |
| 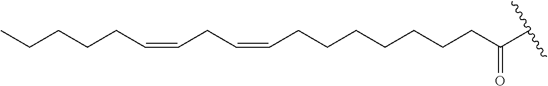 [SMILES: *C(=O)CCCCCCC/C=C\C/C=C\CCCCC] | —C₉H₁₉ |
| 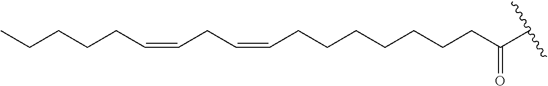 [SMILES: *C(=O)CCCCCCC/C=C\C/C=C\CCCCC] | —C₁₁H₂₃ |
| 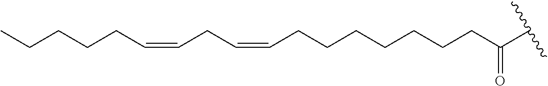 [SMILES: *C(=O)CCCCCCC/C=C\C/C=C\CCCCC] | —C₁₃H₂₇ |
| 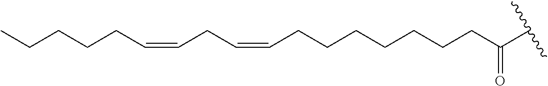 [SMILES: *C(=O)CCCCCCC/C=C\C/C=C\CCCCC] | —C₁₅H₃₁ |
| 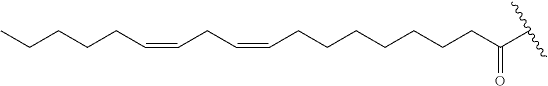 [SMILES: *C(=O)CCCCCCC/C=C\C/C=C\CCCCC] | —C₁₇H₃₅ |
| 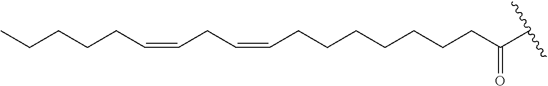 [SMILES: *C(=O)CCCCCCC/C=C\C/C=C\CCCCC] | 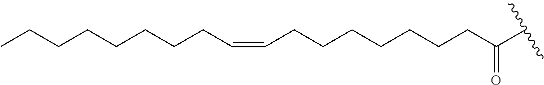 [SMILES: *C(=O)CCCCCCC/C=C\CCCCCCCC] |
| 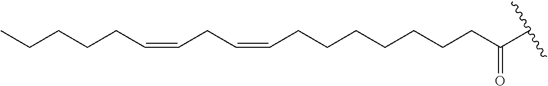 [SMILES: *C(=O)CCCCCCC/C=C\C/C=C\CCCCC] | 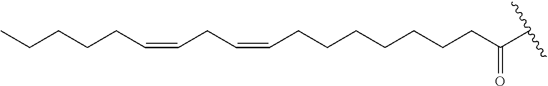 [SMILES: *C(=O)CCCCCCC/C=C\C/C=C\CCCCC] |
| 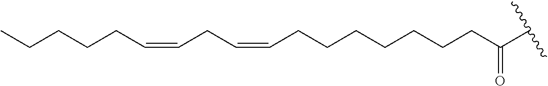 [SMILES: *C(=O)CCCCCCC/C=C\C/C=C\CCCCC] | 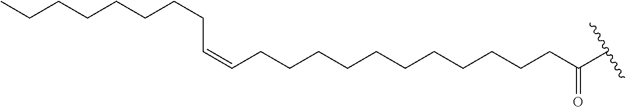 [SMILES: *C(=O)CCCCCCCCCCC/C=C\CCCCCCCC] |
| 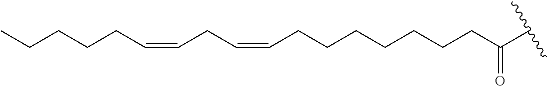 [SMILES: *C(=O)CCCCCCC/C=C\C/C=C\CCCCC] | 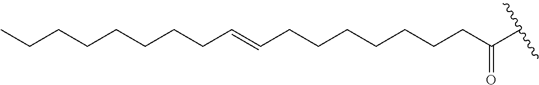 [SMILES: *C(=O)CCCCCCC/C=C/CCCCCCCC] |
| 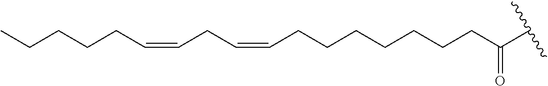 [SMILES: *C(=O)CCCCCCC/C=C\C/C=C\CCCCC] | 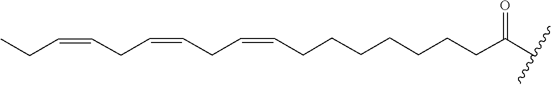 [SMILES: *C(=O)CCCCCCC/C=C\C/C=C\C/C=C\CC] |
| 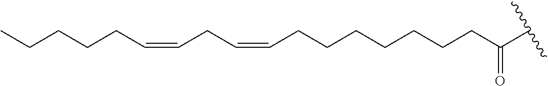 [SMILES: *C(=O)CCCCCCC/C=C\C/C=C\CCCCC] | tBu |
| 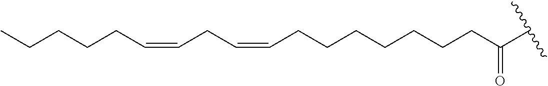 [SMILES: *C(=O)CCCCCCC/C=C\C/C=C\CCCCC] | phenyl |
| 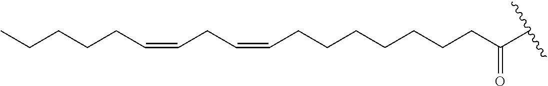 [SMILES: *C(=O)CCCCCCC/C=C\C/C=C\CCCCC] | 4-chlorophenyl |
| 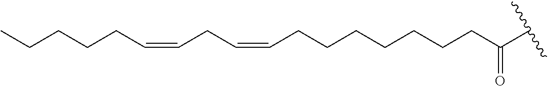 [SMILES: *C(=O)CCCCCCC/C=C\C/C=C\CCCCC] | 3,4-dichlorophenyl |
| 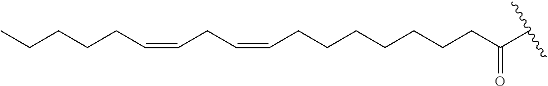 [SMILES: *C(=O)CCCCCCC/C=C\C/C=C\CCCCC] | 4-methylphenyl |
| 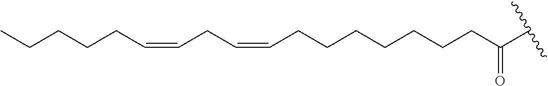 [SMILES: *C(=O)CCCCCCC/C=C\C/C=C\CCCCC] | 4-methoxyphenyl |
| 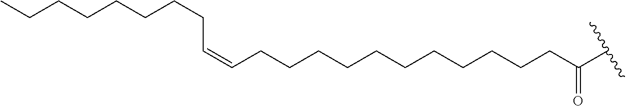 [SMILES: *C(=O)CCCCCCCCCCC/C=C\CCCCCCCC] | Me |
| 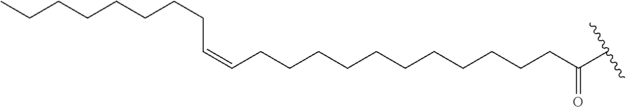 [SMILES: *C(=O)CCCCCCCCCCC/C=C\CCCCCCCC] | —C₇H₁₅ |
| 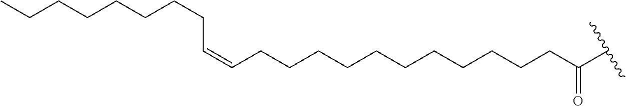 [SMILES: *C(=O)CCCCCCCCCCC/C=C\CCCCCCCC] | —C₉H₁₉ |
| 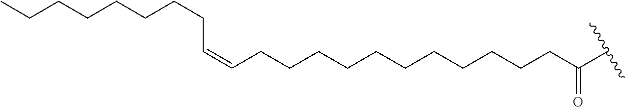 [SMILES: *C(=O)CCCCCCCCCCC/C=C\CCCCCCCC] | —C₁₁H₂₃ |
| 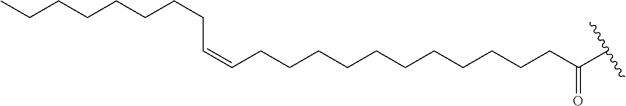 [SMILES: *C(=O)CCCCCCCCCCC/C=C\CCCCCCCC] | —C₁₃H₂₇ |
| 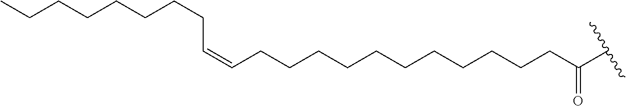 [SMILES: *C(=O)CCCCCCCCCCC/C=C\CCCCCCCC] | —C₁₅H₃₁ |
| 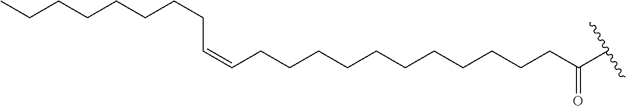 [SMILES: *C(=O)CCCCCCCCCCC/C=C\CCCCCCCC] | —C₁₇H₃₅ |
| 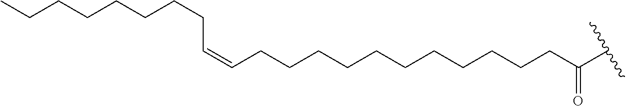 [SMILES: *C(=O)CCCCCCCCCCC/C=C\CCCCCCCC] | 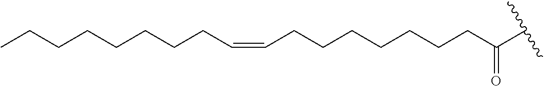 [SMILES: *C(=O)CCCCCCC/C=C\CCCCCCCC] |
| 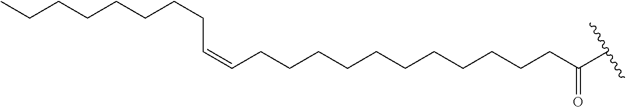 [SMILES: *C(=O)CCCCCCCCCCC/C=C\CCCCCCCC] | 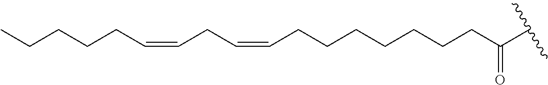 [SMILES: *C(=O)CCCCCCC/C=C\C/C=C\CCCCC] |
| 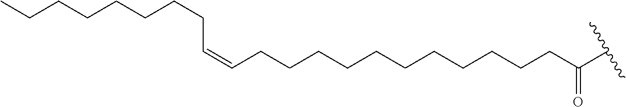 [SMILES: *C(=O)CCCCCCCCCCC/C=C\CCCCCCCC] | 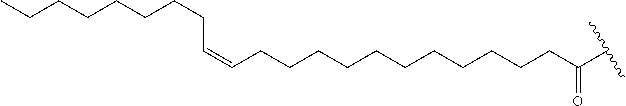 [SMILES: *C(=O)CCCCCCCCCCC/C=C\CCCCCCCC] |
| 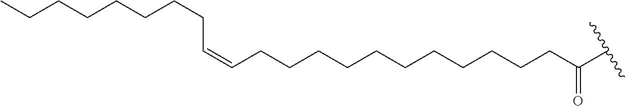 [SMILES: *C(=O)CCCCCCCCCCC/C=C\CCCCCCCC] | 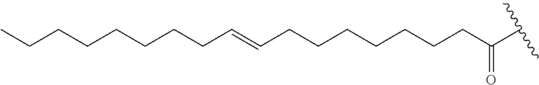 [SMILES: *C(=O)CCCCCCC/C=C/CCCCCCCC] |
| 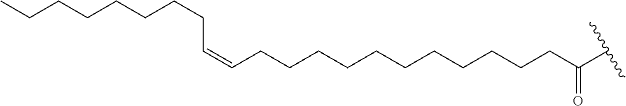 [SMILES: *C(=O)CCCCCCCCCCC/C=C\CCCCCCCC] | 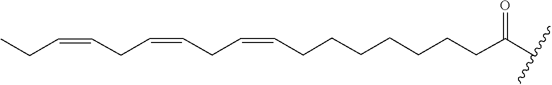 [SMILES: *C(=O)CCCCCCC/C=C\C/C=C\C/C=C\CC] |
| 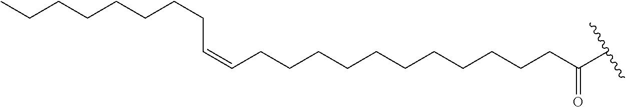 [SMILES: *C(=O)CCCCCCCCCCC/C=C\CCCCCCCC] | tBu |
| 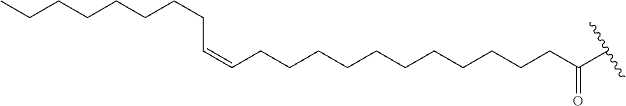 [SMILES: *C(=O)CCCCCCCCCCC/C=C\CCCCCCCC] | phenyl |
| 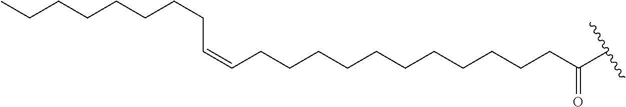 [SMILES: *C(=O)CCCCCCCCCCC/C=C\CCCCCCCC] | 4-chlorophenyl |
| 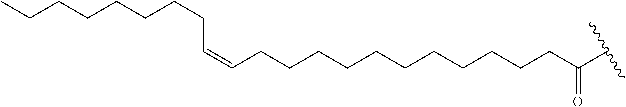 [SMILES: *C(=O)CCCCCCCCCCC/C=C\CCCCCCCC] | 3,4-dichlorophenyl |
| 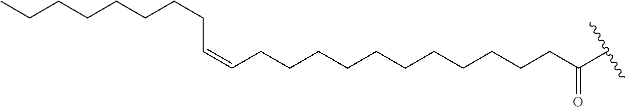 [SMILES: *C(=O)CCCCCCCCCCC/C=C\CCCCCCCC] | 4-methylphenyl |
| 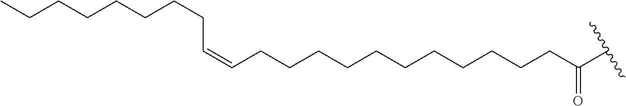 [SMILES: *C(=O)CCCCCCCCCCC/C=C\CCCCCCCC] | 4-methoxyphenyl |
| 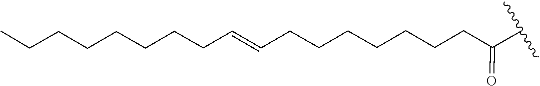 [SMILES: *C(=O)CCCCCCC/C=C/CCCCCCCC] | Me |
| 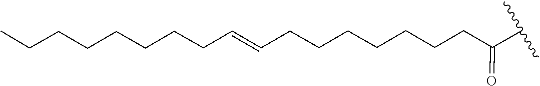 [SMILES: *C(=O)CCCCCCC/C=C/CCCCCCCC] | —C₇H₁₅ |
| 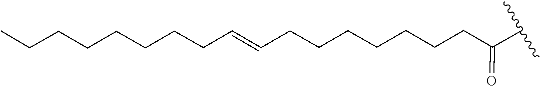 [SMILES: *C(=O)CCCCCCC/C=C/CCCCCCCC] | —C₉H₁₉ |
| 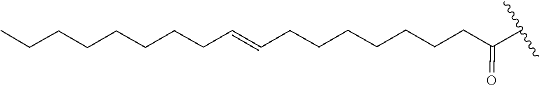 [SMILES: *C(=O)CCCCCCC/C=C/CCCCCCCC] | —C₁₁H₂₃ |
| 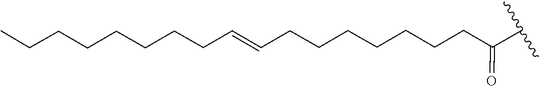 [SMILES: *C(=O)CCCCCCC/C=C/CCCCCCCC] | —C₁₃H₂₇ |
| 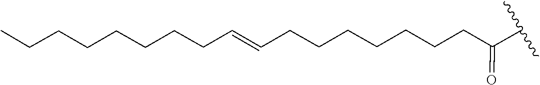 [SMILES: *C(=O)CCCCCCC/C=C/CCCCCCCC] | —C₁₅H₃₁ |
| 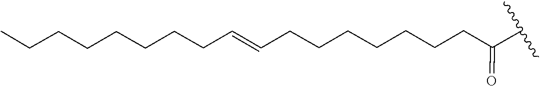 [SMILES: *C(=O)CCCCCCC/C=C/CCCCCCCC] | —C₁₇H₃₅ |
| 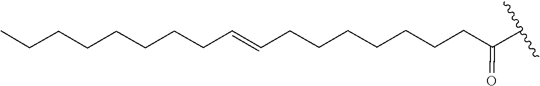 [SMILES: *C(=O)CCCCCCC/C=C/CCCCCCCC] | 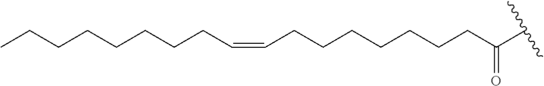 [SMILES: *C(=O)CCCCCCC/C=C\CCCCCCCC] |
| 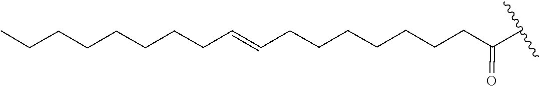 [SMILES: *C(=O)CCCCCCC/C=C/CCCCCCCC] | 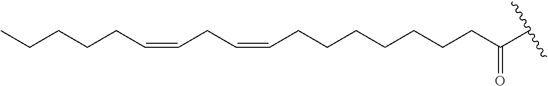 [SMILES: *C(=O)CCCCCCC/C=C\C/C=C\CCCCC] |
| 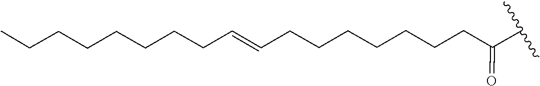 [SMILES: *C(=O)CCCCCCC/C=C/CCCCCCCC] | 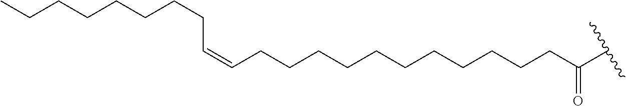 [SMILES: *C(=O)CCCCCCCCCCC/C=C\CCCCCCCC] |
| 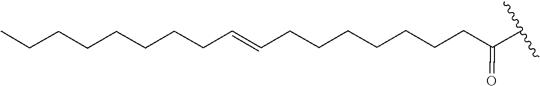 [SMILES: *C(=O)CCCCCCC/C=C/CCCCCCCC] | 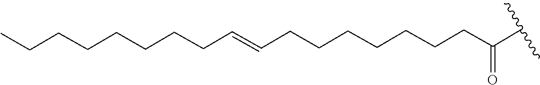 [SMILES: *C(=O)CCCCCCC/C=C/CCCCCCCC] |
| 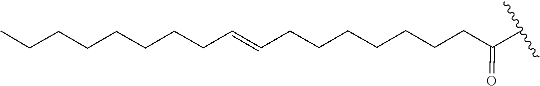 [SMILES: *C(=O)CCCCCCC/C=C/CCCCCCCC] | 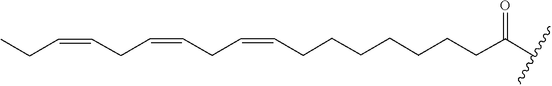 [SMILES: *C(=O)CCCCCCC/C=C\C/C=C\C/C=C\CC] |
| 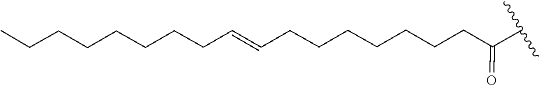 [SMILES: *C(=O)CCCCCCC/C=C/CCCCCCCC] | tBu |
| 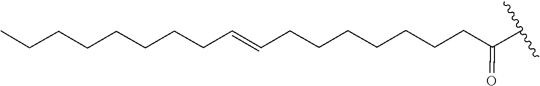 [SMILES: *C(=O)CCCCCCC/C=C/CCCCCCCC] | phenyl |
| 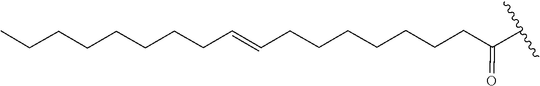 [SMILES: *C(=O)CCCCCCC/C=C/CCCCCCCC] | 4-chlorophenyl |
| 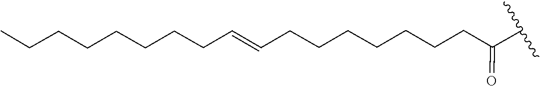 [SMILES: *C(=O)CCCCCCC/C=C/CCCCCCCC] | 3,4-dichlorophenyl |
| 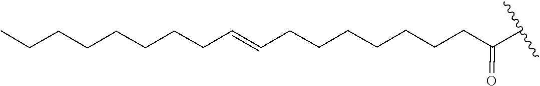 [SMILES: *C(=O)CCCCCCC/C=C/CCCCCCCC] | 4-methylphenyl |
| 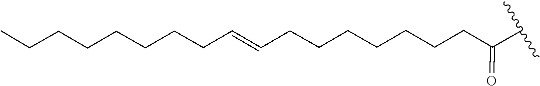 [SMILES: *C(=O)CCCCCCC/C=C/CCCCCCCC] | 4-methoxyphenyl |
| 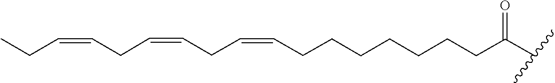 [SMILES: *C(=O)CCCCCCC/C=C\C/C=C\C/C=C\CC] | Me |
| 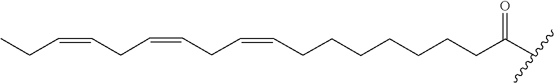 [SMILES: *C(=O)CCCCCCC/C=C\C/C=C\C/C=C\CC] | —C₇H₁₅ |
| 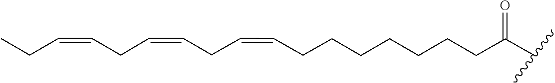 [SMILES: *C(=O)CCCCCCC/C=C\C/C=C\C/C=C\CC] | —C₉H₁₉ |
| 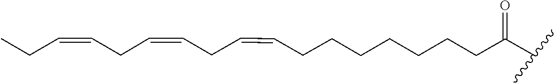 [SMILES: *C(=O)CCCCCCC/C=C\C/C=C\C/C=C\CC] | —C₁₁H₂₃ |
| 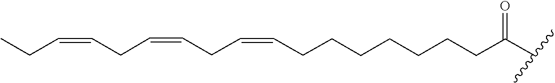 [SMILES: *C(=O)CCCCCCC/C=C\C/C=C\C/C=C\CC] | —C₁₃H₂₇ |
| 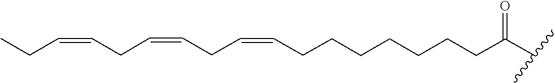 [SMILES: *C(=O)CCCCCCC/C=C\C/C=C\C/C=C\CC] | —C₁₅H₃₁ |
| 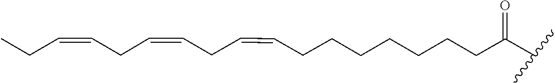 [SMILES: *C(=O)CCCCCCC/C=C\C/C=C\C/C=C\CC] | —C₁₇H₃₅ |
| 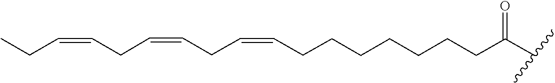 [SMILES: *C(=O)CCCCCCC/C=C\C/C=C\C/C=C\CC] | 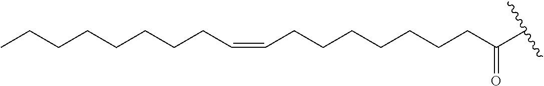 [SMILES: *C(=O)CCCCCCC/C=C\CCCCCCCC] |
| 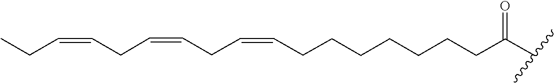 [SMILES: *C(=O)CCCCCCC/C=C\C/C=C\C/C=C\CC] | 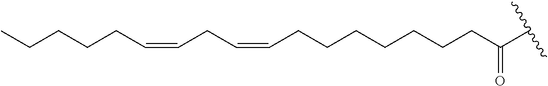 [SMILES: *C(=O)CCCCCCC/C=C\C/C=C\CCCCC] |
| 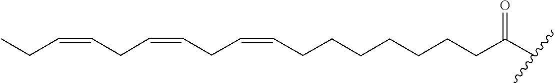 [SMILES: *C(=O)CCCCCCC/C=C\C/C=C\C/C=C\CC] | 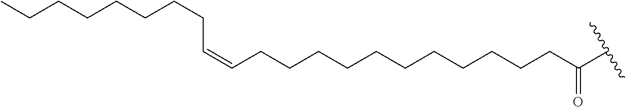 [SMILES: *C(=O)CCCCCCCCCCC/C=C\CCCCCCCC] |
| 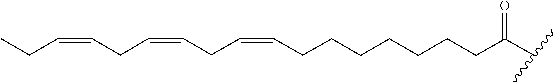 [SMILES: *C(=O)CCCCCCC/C=C\C/C=C\C/C=C\CC] | 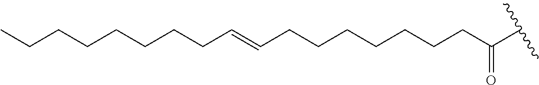 [SMILES: *C(=O)CCCCCCC/C=C/CCCCCCCC] |
| 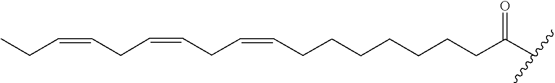 [SMILES: *C(=O)CCCCCCC/C=C\C/C=C\C/C=C\CC] | 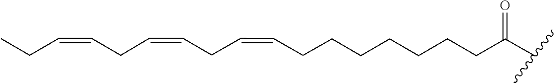 [SMILES: *C(=O)CCCCCCC/C=C\C/C=C\C/C=C\CC] |
| 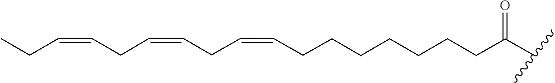 [SMILES: *C(=O)CCCCCCC/C=C\C/C=C\C/C=C\CC] | tBu |
| 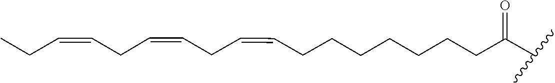 [SMILES: *C(=O)CCCCCCC/C=C\C/C=C\C/C=C\CC] | phenyl |
| 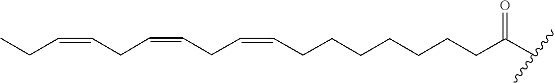 [SMILES: *C(=O)CCCCCCC/C=C\C/C=C\C/C=C\CC] | 4-chlorophenyl |
| 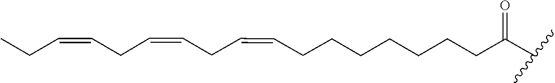 [SMILES: *C(=O)CCCCCCC/C=C\C/C=C\C/C=C\CC] | 3,4-dichlorophenyl |
| 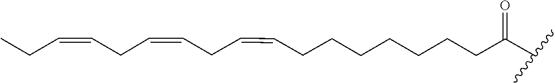 [SMILES: *C(=O)CCCCCCC/C=C\C/C=C\C/C=C\CC] | 4-methylphenyl |
| 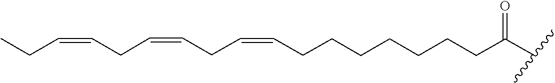 [SMILES: *C(=O)CCCCCCC/C=C\C/C=C\C/C=C\CC] | 4-methoxyphenyl |
| H | 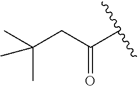 [SMILES: *C(=O)CC(C)(C)C] |
| H | 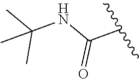 [SMILES: *C(=O)NC(C)(C)C] |
| H | 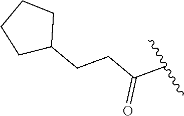 [SMILES: *C(=O)CCC1CCCC1] |
| H | 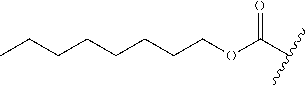 [SMILES: *C(=O)OCCCCCCCC] |
| H | 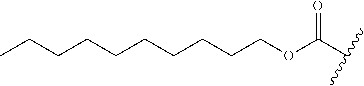 [SMILES: *C(=O)OCCCCCCCCCC] |
| H | 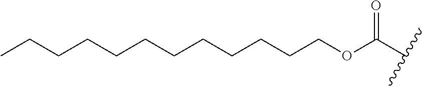 [SMILES: *C(=O)OCCCCCCCCCCCC] |
| H | 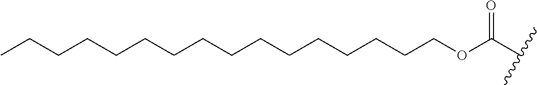 [SMILES: *C(=O)OCCCCCCCCCCCCCCCC] |
| H | 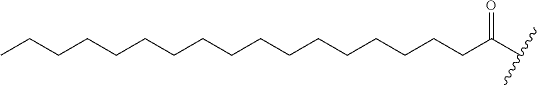 [SMILES: *C(=O)CCCCCCCCCCCCCCCCC] |
| H | 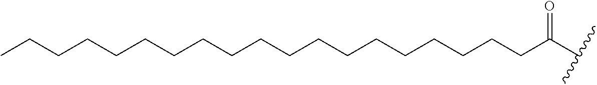 [SMILES: *C(=O)CCCCCCCCCCCCCCCCCCC] |
| H | 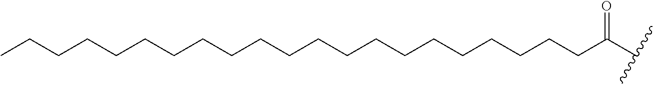 [SMILES: *C(=O)CCCCCCCCCCCCCCCCCCCCC] |
| H | 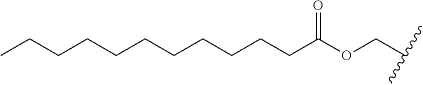 [SMILES: *COC(=O)CCCCCCCCCCC] |
| H | 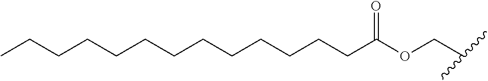 [SMILES: *COC(=O)CCCCCCCCCCCCC] |
| H | 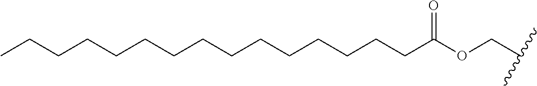 [SMILES: *COC(=O)CCCCCCCCCCCCCCC] |
| H | 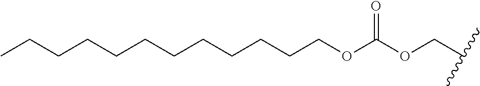 [SMILES: *COC(=O)OCCCCCCCCCCCC] |
| H | 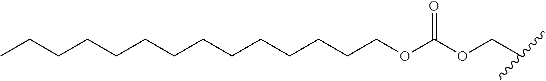 [SMILES: *COC(=O)OCCCCCCCCCCCCCC] |
| H | 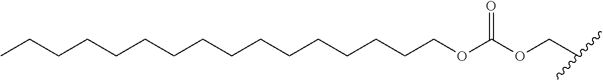 [SMILES: *COC(=O)OCCCCCCCCCCCCCCCC] |
| Me | 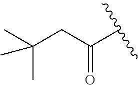 [SMILES: *C(=O)CC(C)(C)C] |
| Me | 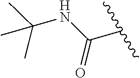 [SMILES: *C(=O)NC(C)(C)C] |
| Me | 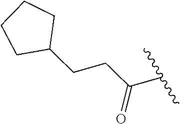 [SMILES: *C(=O)CCC1CCCC1] |
| Me | 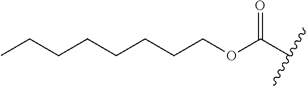 [SMILES: *C(=O)OCCCCCCCC] |
| Me | 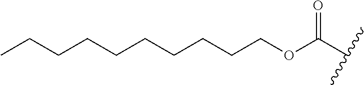 [SMILES: *C(=O)OCCCCCCCCCC] |
| Me | 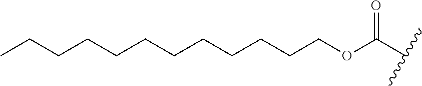 [SMILES: *C(=O)OCCCCCCCCCCCC] |
| Me | 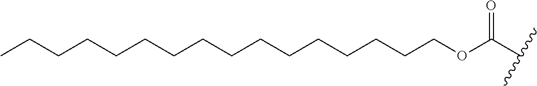 [SMILES: *C(=O)OCCCCCCCCCCCCCCCC] |
| Me | 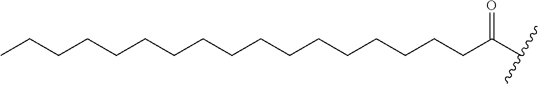 [SMILES: *C(=O)CCCCCCCCCCCCCCCCC] |
| Me | 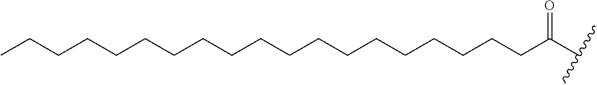 [SMILES: *C(=O)CCCCCCCCCCCCCCCCCCC] |
| Me | 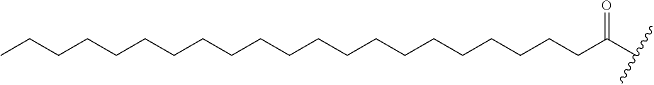 [SMILES: *C(=O)CCCCCCCCCCCCCCCCCCCCC] |
| Me | 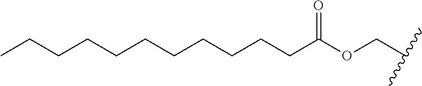 [SMILES: *COC(=O)CCCCCCCCCCC] |
| Me | 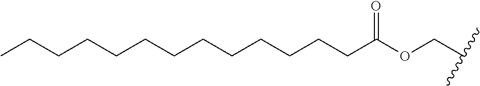 [SMILES: *COC(=O)CCCCCCCCCCCCC] |
| Me | 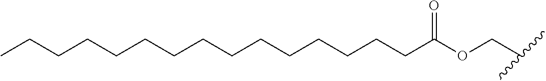 [SMILES: *COC(=O)CCCCCCCCCCCCCCC] |
| Me | 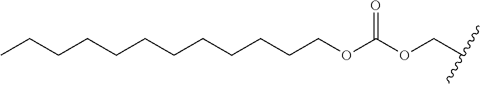 [SMILES: *COC(=O)OCCCCCCCCCCCC] |
| Me | 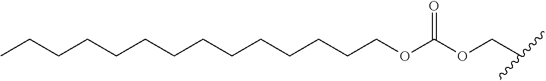 [SMILES: *COC(=O)OCCCCCCCCCCCCCC] |
| Me | 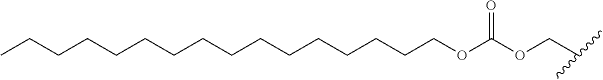 [SMILES: *COC(=O)OCCCCCCCCCCCCCCCC] |
| —C₇H₁₅ | 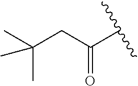 [SMILES: *C(=O)CC(C)(C)C] |
| —C₇H₁₅ | 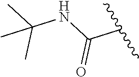 [SMILES: *C(=O)NC(C)(C)C] |
| —C₇H₁₅ | 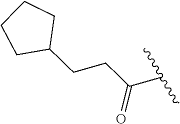 [SMILES: *C(=O)CCC1CCCC1] |
| —C₇H₁₅ | 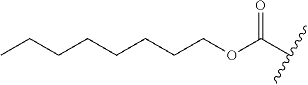 [SMILES: *C(=O)OCCCCCCCC] |
| —C₇H₁₅ | 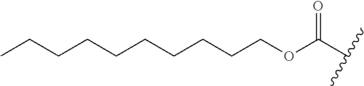 [SMILES: *C(=O)OCCCCCCCCCC] |
| —C₇H₁₅ | 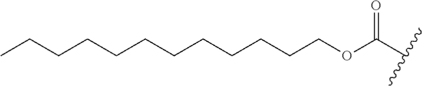 [SMILES: *C(=O)OCCCCCCCCCCCC] |
| —C₇H₁₅ | 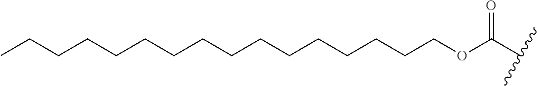 [SMILES: *C(=O)OCCCCCCCCCCCCCCCC] |
| —C₇H₁₅ | 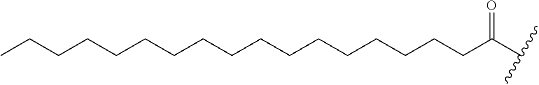 [SMILES: *C(=O)CCCCCCCCCCCCCCCCC] |
| —C₇H₁₅ | 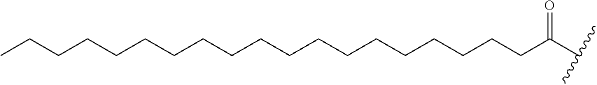 [SMILES: *C(=O)CCCCCCCCCCCCCCCCCCC] |
| —C₇H₁₅ | 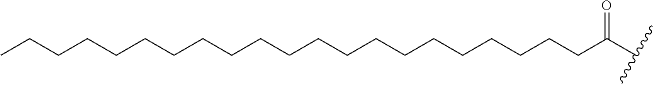 [SMILES: *C(=O)CCCCCCCCCCCCCCCCCCCCC] |
| —C₇H₁₅ | 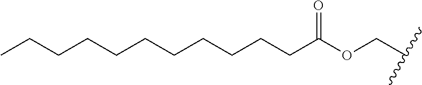 [SMILES: *COC(=O)CCCCCCCCCCC] |
| —C₇H₁₅ | 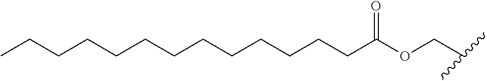 [SMILES: *COC(=O)CCCCCCCCCCCCC] |
| —C₇H₁₅ | 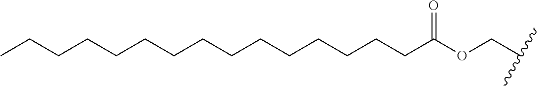 [SMILES: *COC(=O)CCCCCCCCCCCCCCC] |
| —C₇H₁₅ | 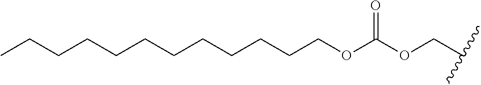 [SMILES: *COC(=O)OCCCCCCCCCCCC] |
| —C₇H₁₅ | 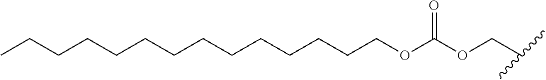 [SMILES: *COC(=O)OCCCCCCCCCCCCCC] |
| —C₇H₁₅ | 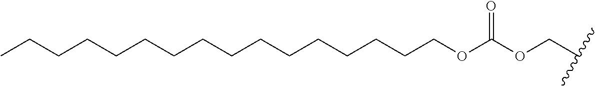 [SMILES: *COC(=O)OCCCCCCCCCCCCCCCC] |
| —C₉H₁₉ | 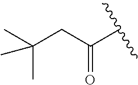 [SMILES: *C(=O)CC(C)(C)C] |
| —C₉H₁₉ | 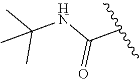 [SMILES: *C(=O)NC(C)(C)C] |
| —C₉H₁₉ | 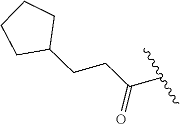 [SMILES: *C(=O)CCC1CCCC1] |
| —C₉H₁₉ | 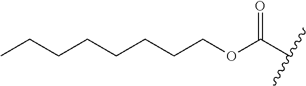 [SMILES: *C(=O)OCCCCCCCC] |
| —C₉H₁₉ | 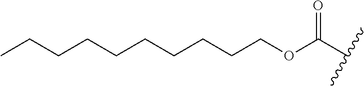 [SMILES: *C(=O)OCCCCCCCCCC] |
| —C₉H₁₉ | 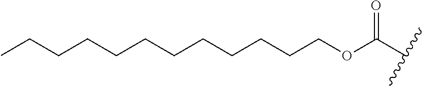 [SMILES: *C(=O)OCCCCCCCCCCCC] |
| —C₉H₁₉ | 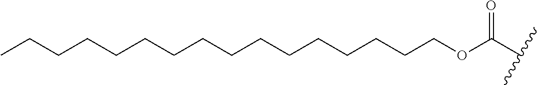 [SMILES: *C(=O)OCCCCCCCCCCCCCCCC] |
| —C₉H₁₉ | 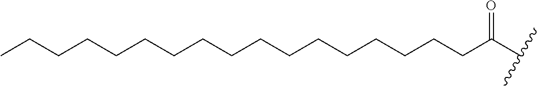 [SMILES: *C(=O)CCCCCCCCCCCCCCCCC] |
| —C₉H₁₉ | 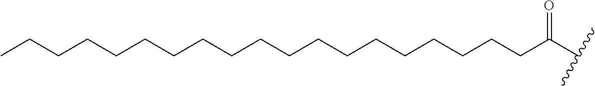 [SMILES: *C(=O)CCCCCCCCCCCCCCCCCCC] |
| —C₉H₁₉ | 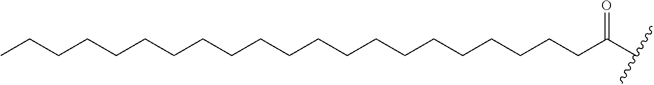 [SMILES: *C(=O)CCCCCCCCCCCCCCCCCCCCC] |
| —C₉H₁₉ | 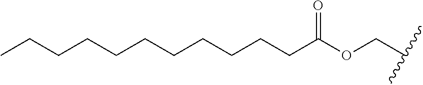 [SMILES: *COC(=O)CCCCCCCCCCC] |
| —C₉H₁₉ | 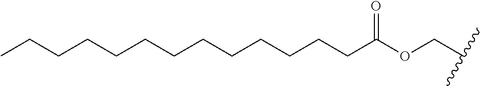 [SMILES: *COC(=O)CCCCCCCCCCCCC] |
| —C₉H₁₉ | 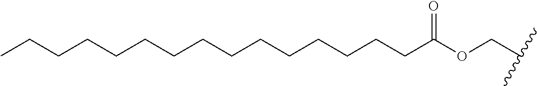 [SMILES: *COC(=O)CCCCCCCCCCCCCCC] |
| —C₉H₁₉ | 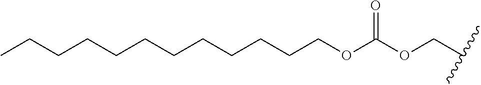 [SMILES: *COC(=O)OCCCCCCCCCCCC] |
| —C₉H₁₉ | 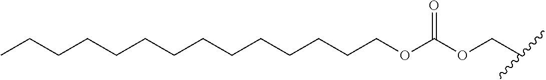 [SMILES: *COC(=O)OCCCCCCCCCCCCCC] |
| —C₉H₁₉ | 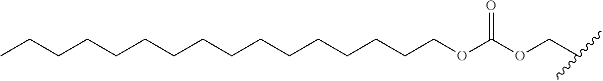 [SMILES: *COC(=O)OCCCCCCCCCCCCCCCC] |
| —C₁₁H₂₃ | 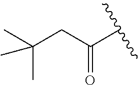 [SMILES: *C(=O)CC(C)(C)C] |
| —C₁₁H₂₃ | 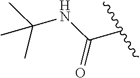 [SMILES: *C(=O)NC(C)(C)C] |
| —C₁₁H₂₃ | 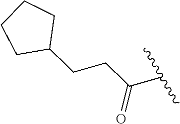 [SMILES: *C(=O)CCC1CCCC1] |
| —C₁₁H₂₃ | 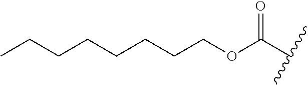 [SMILES: *C(=O)OCCCCCCCC] |
| —C₁₁H₂₃ | 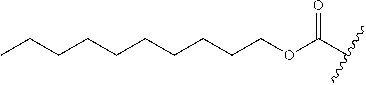 [SMILES: *C(=O)OCCCCCCCCCC] |
| —C₁₁H₂₃ | 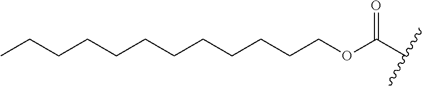 [SMILES: *C(=O)OCCCCCCCCCCCC] |
| —C₁₁H₂₃ | 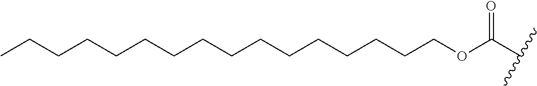 [SMILES: *C(=O)OCCCCCCCCCCCCCCCC] |
| —C₁₁H₂₃ | 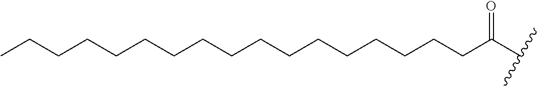 [SMILES: *C(=O)CCCCCCCCCCCCCCCCC] |
| —C₁₁H₂₃ | 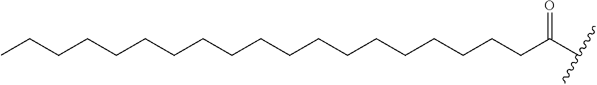 [SMILES: *C(=O)CCCCCCCCCCCCCCCCCCC] |
| —C₁₁H₂₃ | 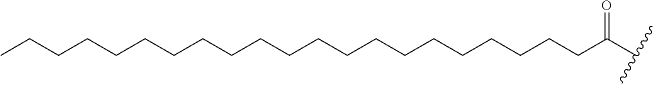 [SMILES: *C(=O)CCCCCCCCCCCCCCCCCCCCC] |
| —C₁₁H₂₃ | 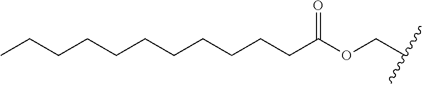 [SMILES: *COC(=O)CCCCCCCCCCC] |
| —C₁₁H₂₃ | 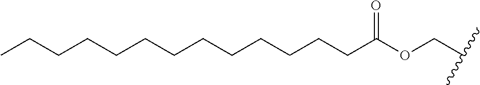 [SMILES: *COC(=O)CCCCCCCCCCCCC] |
| —C₁₁H₂₃ | 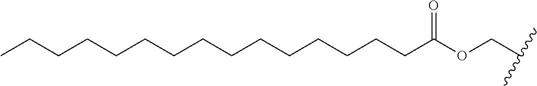 [SMILES: *COC(=O)CCCCCCCCCCCCCCC] |
| —C₁₁H₂₃ | 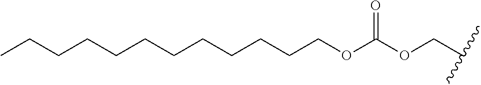 [SMILES: *COC(=O)OCCCCCCCCCCCC] |
| —C₁₁H₂₃ | 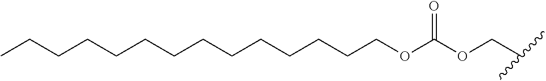 [SMILES: *COC(=O)OCCCCCCCCCCCCCC] |
| —C₁₁H₂₃ | 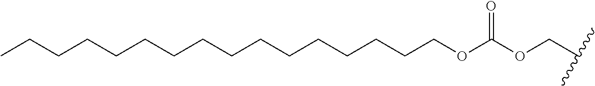 [SMILES: *COC(=O)OCCCCCCCCCCCCCCCC] |
| —C₁₃H₂₇ | 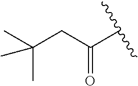 [SMILES: *C(=O)CC(C)(C)C] |
| —C₁₃H₂₇ | 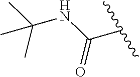 [SMILES: *C(=O)NC(C)(C)C] |
| —C₁₃H₂₇ | 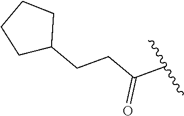 [SMILES: *C(=O)CCC1CCCC1] |
| —C₁₃H₂₇ | 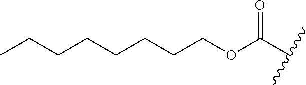 [SMILES: *C(=O)OCCCCCCCC] |
| —C₁₃H₂₇ | 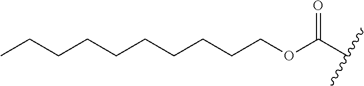 [SMILES: *C(=O)OCCCCCCCCCC] |
| —C₁₃H₂₇ | 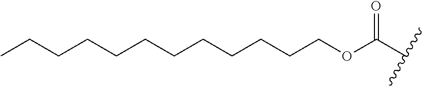 [SMILES: *C(=O)OCCCCCCCCCCCC] |
| —C₁₃H₂₇ | 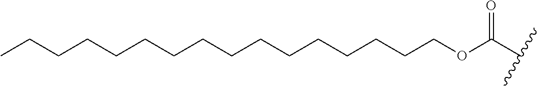 [SMILES: *C(=O)OCCCCCCCCCCCCCCCC] |
| —C₁₃H₂₇ | 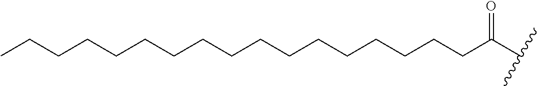 [SMILES: *C(=O)CCCCCCCCCCCCCCCCC] |
| —C₁₃H₂₇ | 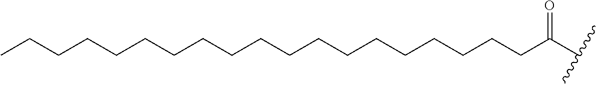 [SMILES: *C(=O)CCCCCCCCCCCCCCCCCCC] |
| —C₁₃H₂₇ | 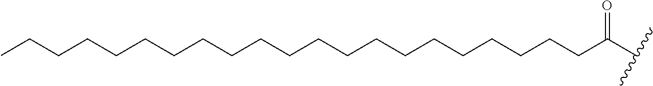 [SMILES: *C(=O)CCCCCCCCCCCCCCCCCCCCC] |
| —C₁₃H₂₇ | 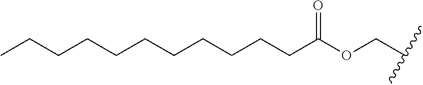 [SMILES: *COC(=O)CCCCCCCCCCC] |
| —C₁₃H₂₇ | 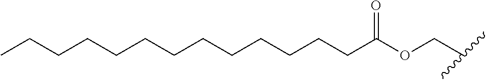 [SMILES: *COC(=O)CCCCCCCCCCCCC] |
| —C₁₃H₂₇ | 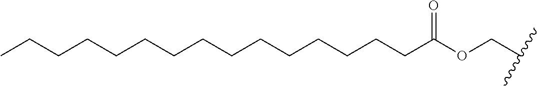 [SMILES: *COC(=O)CCCCCCCCCCCCCCC] |
| —C₁₃H₂₇ | 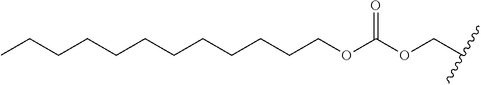 [SMILES: *COC(=O)OCCCCCCCCCCCC] |
| —C₁₃H₂₇ | 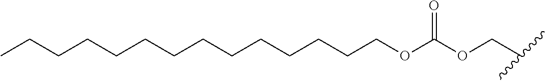 [SMILES: *COC(=O)OCCCCCCCCCCCCCC] |
| —C₁₃H₂₇ | 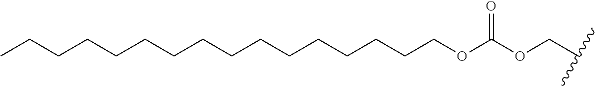 [SMILES: *COC(=O)OCCCCCCCCCCCCCCCC] |
| —C₁₅H₃₁ | 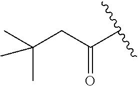 [SMILES: *C(=O)CC(C)(C)C] |
| —C₁₅H₃₁ | 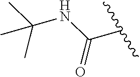 [SMILES: *C(=O)NC(C)(C)C] |
| —C₁₅H₃₁ | 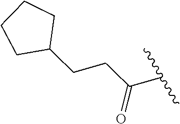 [SMILES: *C(=O)CCC1CCCC1] |
| —C₁₅H₃₁ | 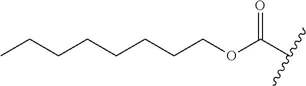 [SMILES: *C(=O)OCCCCCCCC] |
| —C₁₅H₃₁ | 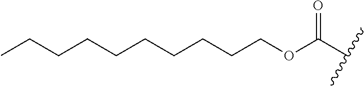 [SMILES: *C(=O)OCCCCCCCCCC] |
| —C₁₅H₃₁ | 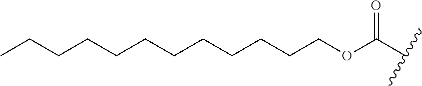 [SMILES: *C(=O)OCCCCCCCCCCCC] |
| —C₁₅H₃₁ | 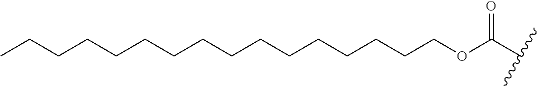 [SMILES: *C(=O)OCCCCCCCCCCCCCCCC] |
| —C₁₅H₃₁ | 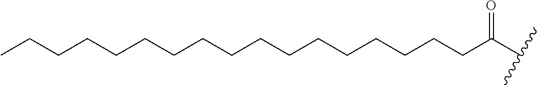 [SMILES: *C(=O)CCCCCCCCCCCCCCCCC] |
| —C₁₅H₃₁ | 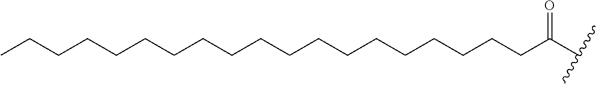 [SMILES: *C(=O)CCCCCCCCCCCCCCCCCCC] |
| —C₁₅H₃₁ | 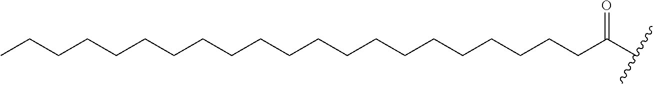 [SMILES: *C(=O)CCCCCCCCCCCCCCCCCCCCC] |
| —C₁₅H₃₁ | 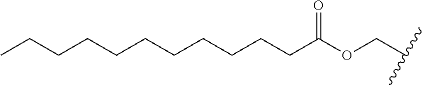 [SMILES: *COC(=O)CCCCCCCCCCC] |
| —C₁₅H₃₁ | 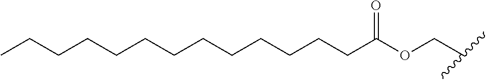 [SMILES: *COC(=O)CCCCCCCCCCCCC] |
| —C₁₅H₃₁ | 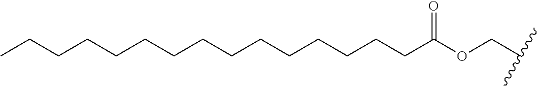 [SMILES: *COC(=O)CCCCCCCCCCCCCCC] |
| —C₁₅H₃₁ | 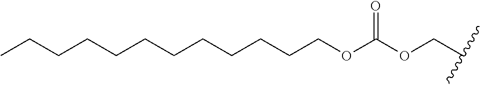 [SMILES: *COC(=O)OCCCCCCCCCCCC] |
| —C₁₅H₃₁ | 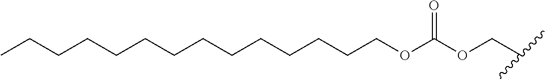 [SMILES: *COC(=O)OCCCCCCCCCCCCCC] |
| —C₁₅H₃₁ | 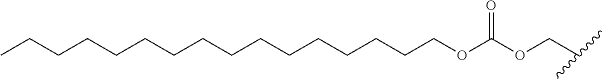 [SMILES: *COC(=O)OCCCCCCCCCCCCCCCC] |
| —C₁₇H₃₅ | 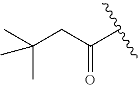 [SMILES: *C(=O)CC(C)(C)C] |
| —C₁₇H₃₅ | 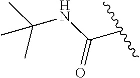 [SMILES: *C(=O)NC(C)(C)C] |
| —C₁₇H₃₅ | 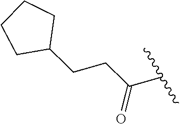 [SMILES: *C(=O)CCC1CCCC1] |
| —C₁₇H₃₅ | 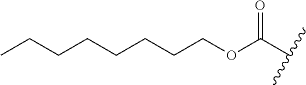 [SMILES: *C(=O)OCCCCCCCC] |
| —C₁₇H₃₅ | 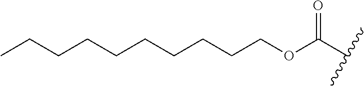 [SMILES: *C(=O)OCCCCCCCCCC] |
| —C₁₇H₃₅ | 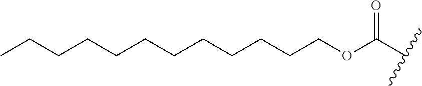 [SMILES: *C(=O)OCCCCCCCCCCCC] |
| —C₁₇H₃₅ | 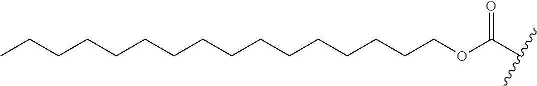 [SMILES: *C(=O)OCCCCCCCCCCCCCCCC] |
| —C₁₇H₃₅ | 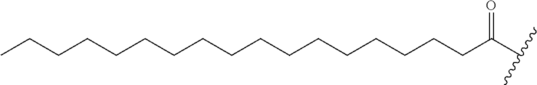 [SMILES: *C(=O)CCCCCCCCCCCCCCCCC] |
| —C₁₇H₃₅ | 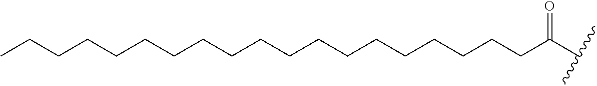 [SMILES: *C(=O)CCCCCCCCCCCCCCCCCCC] |
| —C₁₇H₃₅ | 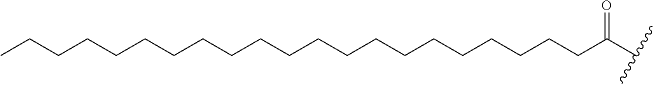 [SMILES: *C(=O)CCCCCCCCCCCCCCCCCCCCC] |
| —C₁₇H₃₅ | 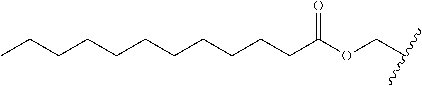 [SMILES: *COC(=O)CCCCCCCCCCC] |
| —C₁₇H₃₅ | 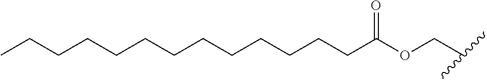 [SMILES: *COC(=O)CCCCCCCCCCCCC] |
| —C₁₇H₃₅ | 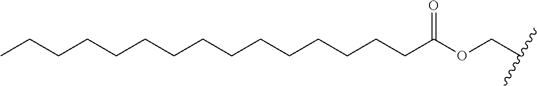 [SMILES: *COC(=O)CCCCCCCCCCCCCCC] |
| —C₁₇H₃₅ | 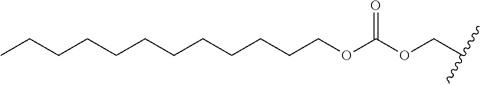 [SMILES: *COC(=O)OCCCCCCCCCCCC] |
| —C₁₇H₃₅ | 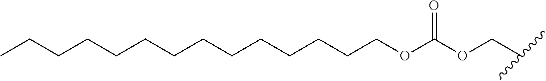 [SMILES: *COC(=O)OCCCCCCCCCCCCCC] |
| —C₁₇H₃₅ | 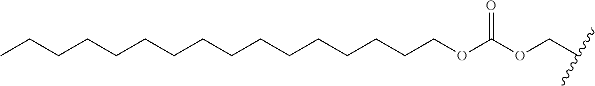 [SMILES: *COC(=O)OCCCCCCCCCCCCCCCC] |
| 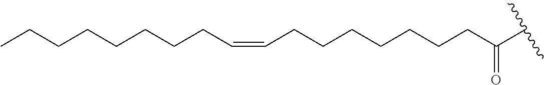 [SMILES: *C(=O)CCCCCCC/C=C\CCCCCCCC] | 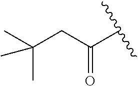 [SMILES: *C(=O)CC(C)(C)C] |
| 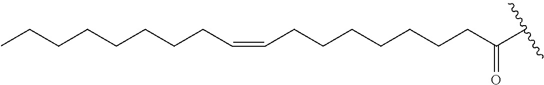 [SMILES: *C(=O)CCCCCCC/C=C\CCCCCCCC] | 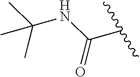 [SMILES: *C(=O)NC(C)(C)C] |
| 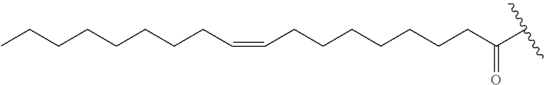 [SMILES: *C(=O)CCCCCCC/C=C\CCCCCCCC] | 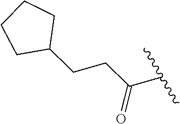 [SMILES: *C(=O)CCC1CCCC1] |
| 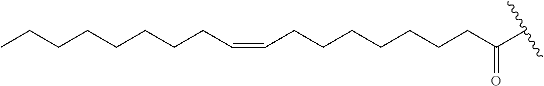 [SMILES: *C(=O)CCCCCCC/C=C\CCCCCCCC] | 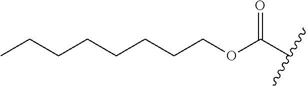 [SMILES: *C(=O)OCCCCCCCC] |
| 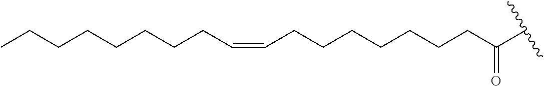 [SMILES: *C(=O)CCCCCCC/C=C\CCCCCCCC] | 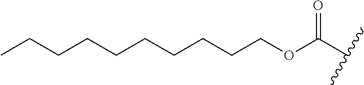 [SMILES: *C(=O)OCCCCCCCCCC] |
| 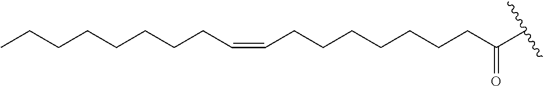 [SMILES: *C(=O)CCCCCCC/C=C\CCCCCCCC] | 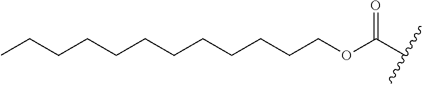 [SMILES: *C(=O)OCCCCCCCCCCCC] |
| 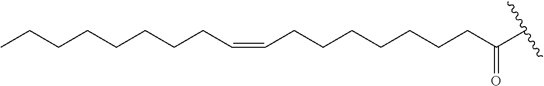 [SMILES: *C(=O)CCCCCCC/C=C\CCCCCCCC] | 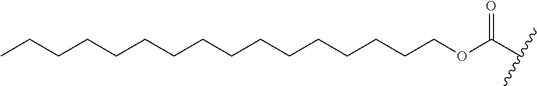 [SMILES: *C(=O)OCCCCCCCCCCCCCCCC] |
| 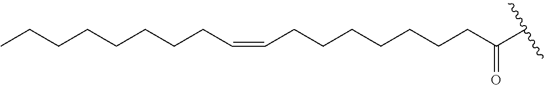 [SMILES: *C(=O)CCCCCCC/C=C\CCCCCCCC] | 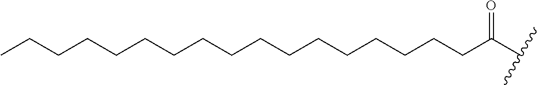 [SMILES: *C(=O)CCCCCCCCCCCCCCCCC] |
| 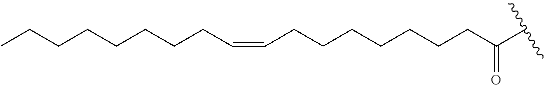 [SMILES: *C(=O)CCCCCCC/C=C\CCCCCCCC] | 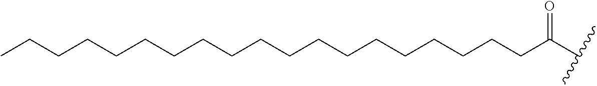 [SMILES: *C(=O)CCCCCCCCCCCCCCCCCCC] |
| 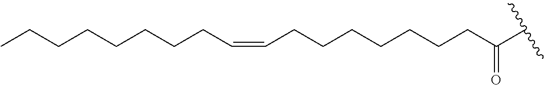 [SMILES: *C(=O)CCCCCCC/C=C\CCCCCCCC] | 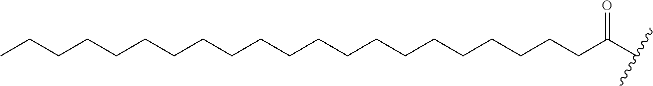 [SMILES: *C(=O)CCCCCCCCCCCCCCCCCCCCC] |
| 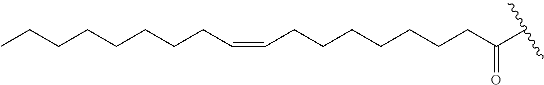 [SMILES: *C(=O)CCCCCCC/C=C\CCCCCCCC] | 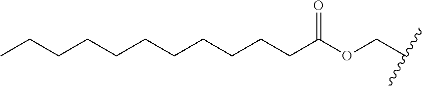 [SMILES: *COC(=O)CCCCCCCCCCC] |
| 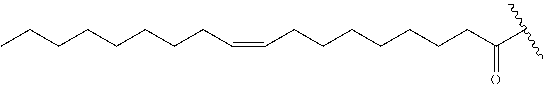 [SMILES: *C(=O)CCCCCCC/C=C\CCCCCCCC] | 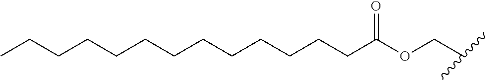 [SMILES: *COC(=O)CCCCCCCCCCCCC] |
| 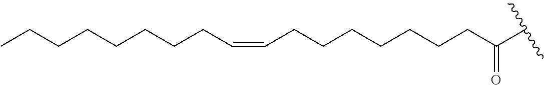 [SMILES: *C(=O)CCCCCCC/C=C\CCCCCCCC] | 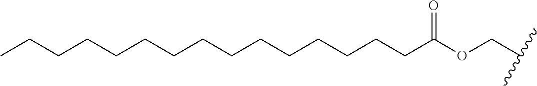 [SMILES: *COC(=O)CCCCCCCCCCCCCCC] |
| 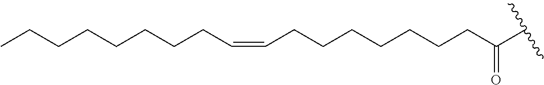 [SMILES: *C(=O)CCCCCCC/C=C\CCCCCCCC] | 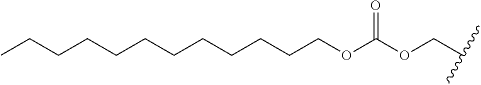 [SMILES: *COC(=O)OCCCCCCCCCCCC] |
| 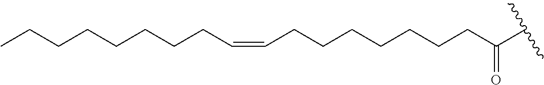 [SMILES: *C(=O)CCCCCCC/C=C\CCCCCCCC] | 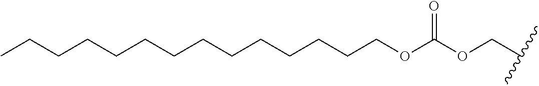 [SMILES: *COC(=O)OCCCCCCCCCCCCCC] |
| 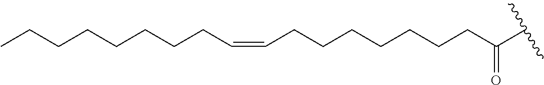 [SMILES: *C(=O)CCCCCCC/C=C\CCCCCCCC] | 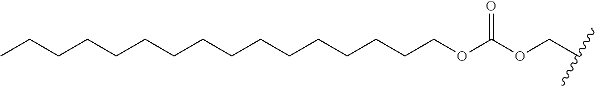 [SMILES: *COC(=O)OCCCCCCCCCCCCCCCC] |
| 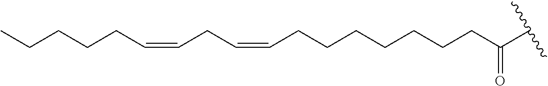 [SMILES: *C(=O)CCCCCCC/C=C\C/C=C\CCCCC] | 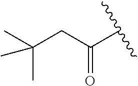 [SMILES: *C(=O)CC(C)(C)C] |
| 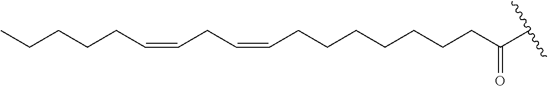 [SMILES: *C(=O)CCCCCCC/C=C\C/C=C\CCCCC] | 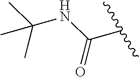 [SMILES: *C(=O)NC(C)(C)C] |
| 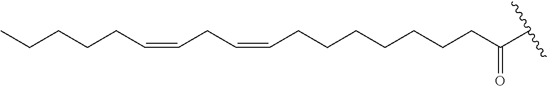 [SMILES: *C(=O)CCCCCCC/C=C\C/C=C\CCCCC] | 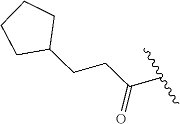 [SMILES: *C(=O)CCC1CCCC1] |
| 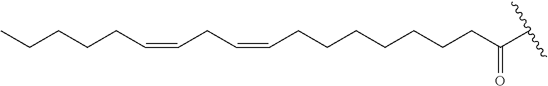 [SMILES: *C(=O)CCCCCCC/C=C\C/C=C\CCCCC] | 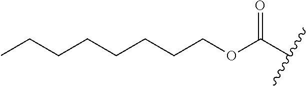 [SMILES: *C(=O)OCCCCCCCC] |
| 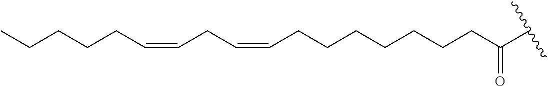 [SMILES: *C(=O)CCCCCCC/C=C\C/C=C\CCCCC] | 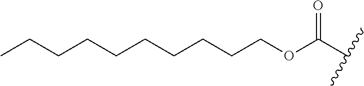 [SMILES: *C(=O)OCCCCCCCCCC] |
| 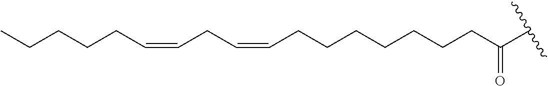 [SMILES: *C(=O)CCCCCCC/C=C\C/C=C\CCCCC] | 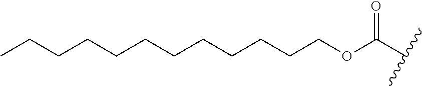 [SMILES: *C(=O)OCCCCCCCCCCCC] |
| 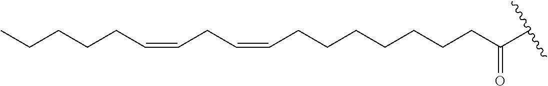 [SMILES: *C(=O)CCCCCCC/C=C\C/C=C\CCCCC] | 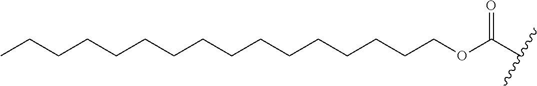 [SMILES: *C(=O)OCCCCCCCCCCCCCCCC] |
| 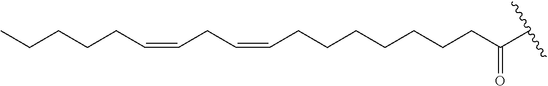 [SMILES: *C(=O)CCCCCCC/C=C\C/C=C\CCCCC] | 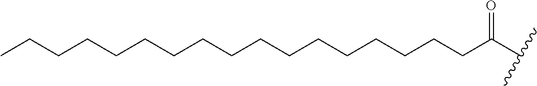 [SMILES: *C(=O)CCCCCCCCCCCCCCCCC] |
| 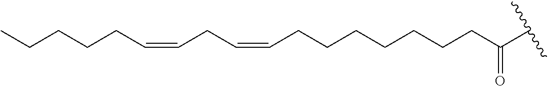 [SMILES: *C(=O)CCCCCCC/C=C\C/C=C\CCCCC] | 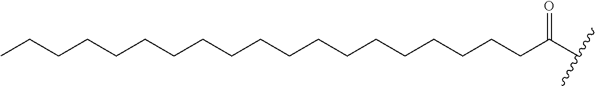 [SMILES: *C(=O)CCCCCCCCCCCCCCCCCCC] |
| 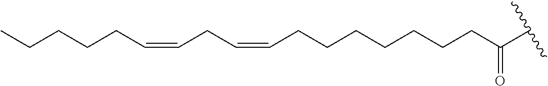 [SMILES: *C(=O)CCCCCCC/C=C\C/C=C\CCCCC] | 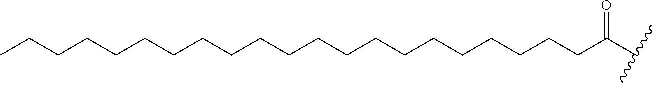 [SMILES: *C(=O)CCCCCCCCCCCCCCCCCCCCC] |
| 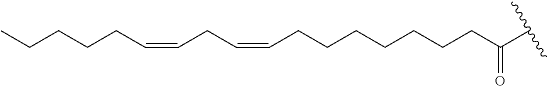 [SMILES: *C(=O)CCCCCCC/C=C\C/C=C\CCCCC] | 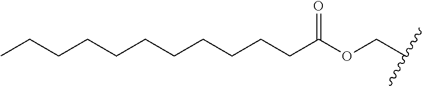 [SMILES: *COC(=O)CCCCCCCCCCC] |
| 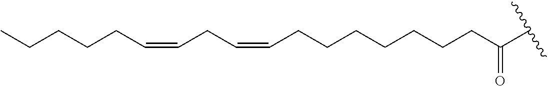 [SMILES: *C(=O)CCCCCCC/C=C\C/C=C\CCCCC] | 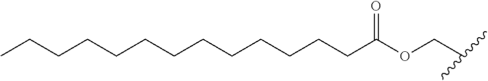 [SMILES: *COC(=O)CCCCCCCCCCCCC] |
| 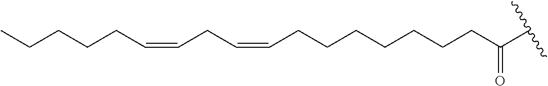 [SMILES: *C(=O)CCCCCCC/C=C\C/C=C\CCCCC] | 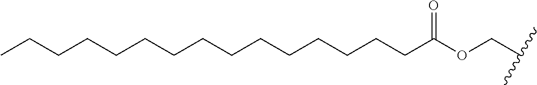 [SMILES: *COC(=O)CCCCCCCCCCCCCCC] |
| 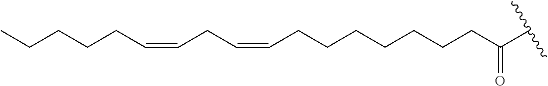 [SMILES: *C(=O)CCCCCCC/C=C\C/C=C\CCCCC] | 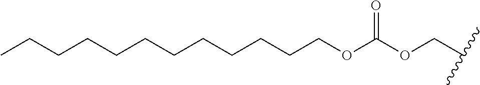 [SMILES: *COC(=O)OCCCCCCCCCCCC] |
| 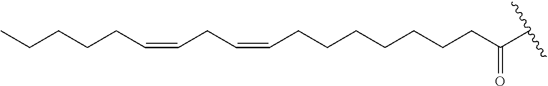 [SMILES: *C(=O)CCCCCCC/C=C\C/C=C\CCCCC] | 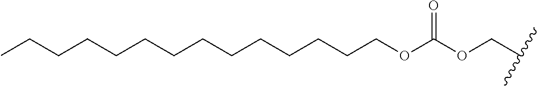 [SMILES: *COC(=O)OCCCCCCCCCCCCCC] |
| 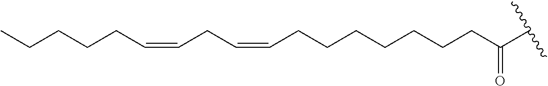 [SMILES: *C(=O)CCCCCCC/C=C\C/C=C\CCCCC] | 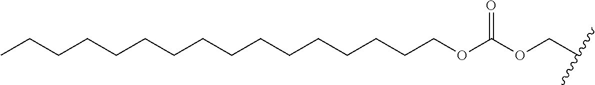 [SMILES: *COC(=O)OCCCCCCCCCCCCCCCC] |
| 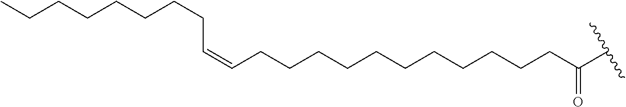 [SMILES: *C(=O)CCCCCCCCCCC/C=C\CCCCCCCC] | 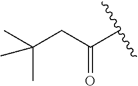 [SMILES: *C(=O)CC(C)(C)C] |
| 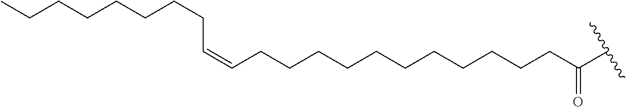 [SMILES: *C(=O)CCCCCCCCCCC/C=C\CCCCCCCC] | 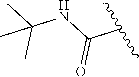 [SMILES: *C(=O)NC(C)(C)C] |
| 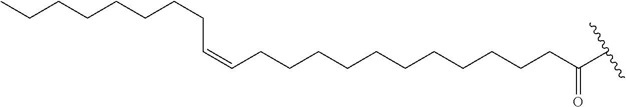 [SMILES: *C(=O)CCCCCCCCCCC/C=C\CCCCCCCC] | 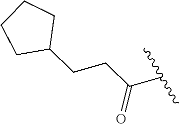 [SMILES: *C(=O)CCC1CCCC1] |
| 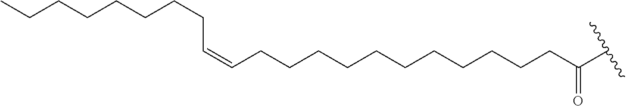 [SMILES: *C(=O)CCCCCCCCCCC/C=C\CCCCCCCC] | 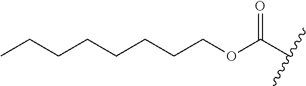 [SMILES: *C(=O)OCCCCCCCC] |
| 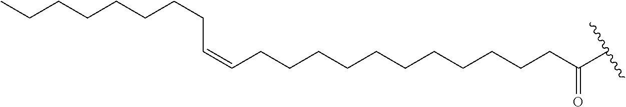 [SMILES: *C(=O)CCCCCCCCCCC/C=C\CCCCCCCC] | 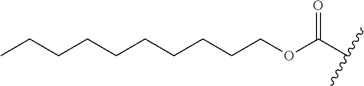 [SMILES: *C(=O)OCCCCCCCCCC] |
| 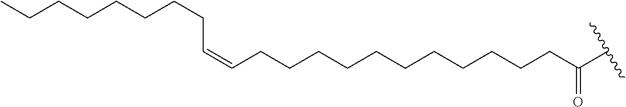 [SMILES: *C(=O)CCCCCCCCCCC/C=C\CCCCCCCC] | 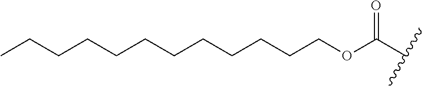 [SMILES: *C(=O)OCCCCCCCCCCCC] |
| 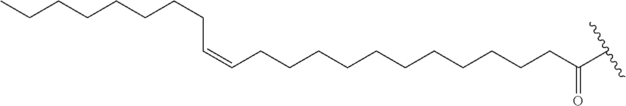 [SMILES: *C(=O)CCCCCCCCCCC/C=C\CCCCCCCC] | 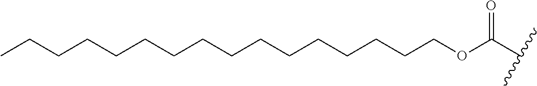 [SMILES: *C(=O)OCCCCCCCCCCCCCCCC] |
| 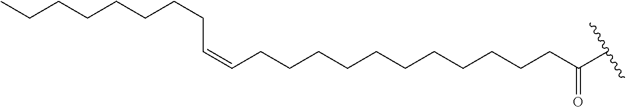 [SMILES: *C(=O)CCCCCCCCCCC/C=C\CCCCCCCC] | 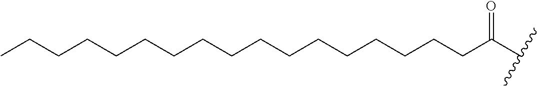 [SMILES: *C(=O)CCCCCCCCCCCCCCCCC] |
| 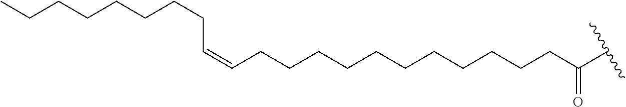 [SMILES: *C(=O)CCCCCCCCCCC/C=C\CCCCCCCC] | 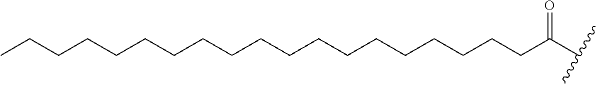 [SMILES: *C(=O)CCCCCCCCCCCCCCCCCCC] |
| 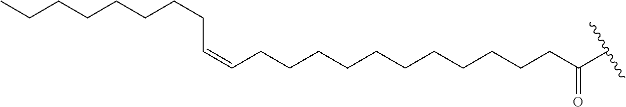 [SMILES: *C(=O)CCCCCCCCCCC/C=C\CCCCCCCC] | 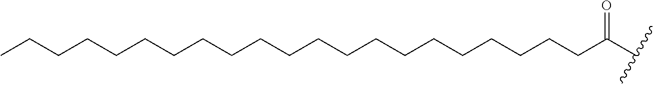 [SMILES: *C(=O)CCCCCCCCCCCCCCCCCCCCC] |
| 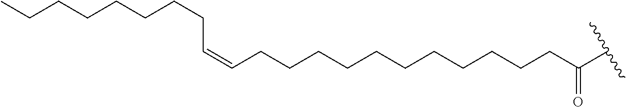 [SMILES: *C(=O)CCCCCCCCCCC/C=C\CCCCCCCC] | 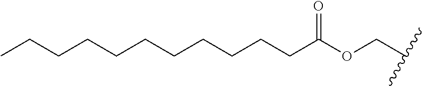 [SMILES: *COC(=O)CCCCCCCCCCC] |
| 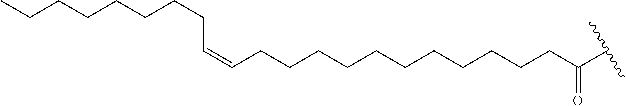 [SMILES: *C(=O)CCCCCCCCCCC/C=C\CCCCCCCC] | 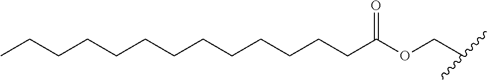 [SMILES: *COC(=O)CCCCCCCCCCCCC] |
| 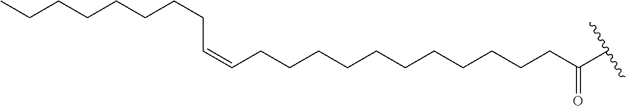 [SMILES: *C(=O)CCCCCCCCCCC/C=C\CCCCCCCC] | 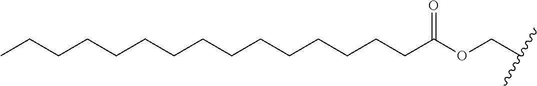 [SMILES: *COC(=O)CCCCCCCCCCCCCCC] |
| 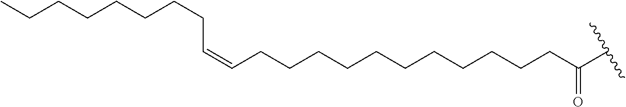 [SMILES: *C(=O)CCCCCCCCCCC/C=C\CCCCCCCC] | 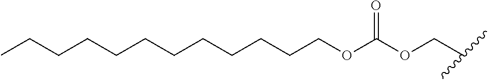 [SMILES: *COC(=O)OCCCCCCCCCCCC] |
| 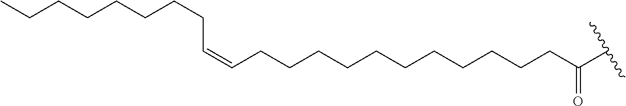 [SMILES: *C(=O)CCCCCCCCCCC/C=C\CCCCCCCC] | 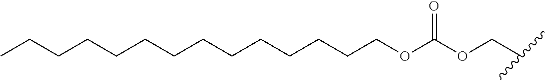 [SMILES: *COC(=O)OCCCCCCCCCCCCCC] |
| 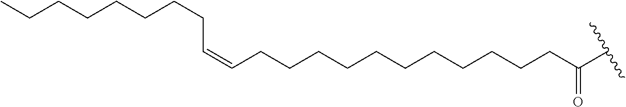 [SMILES: *C(=O)CCCCCCCCCCC/C=C\CCCCCCCC] | 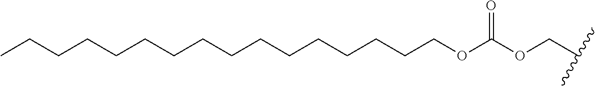 [SMILES: *COC(=O)OCCCCCCCCCCCCCCCC] |
| 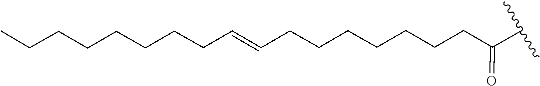 [SMILES: *C(=O)CCCCCCC/C=C/CCCCCCCC] | 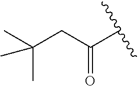 [SMILES: *C(=O)CC(C)(C)C] |
| 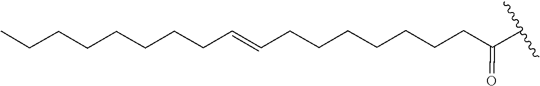 [SMILES: *C(=O)CCCCCCC/C=C/CCCCCCCC] | 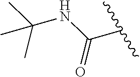 [SMILES: *C(=O)NC(C)(C)C] |
| 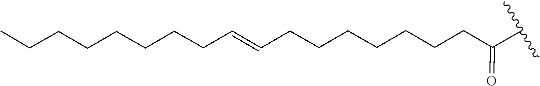 [SMILES: *C(=O)CCCCCCC/C=C/CCCCCCCC] | 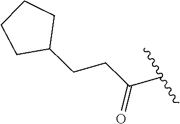 [SMILES: *C(=O)CCC1CCCC1] |
| 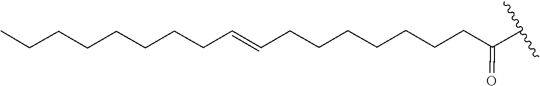 [SMILES: *C(=O)CCCCCCC/C=C/CCCCCCCC] | 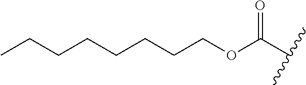 [SMILES: *C(=O)OCCCCCCCC] |
| 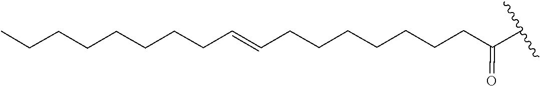 [SMILES: *C(=O)CCCCCCC/C=C/CCCCCCCC] | 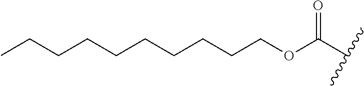 [SMILES: *C(=O)OCCCCCCCCCC] |
| 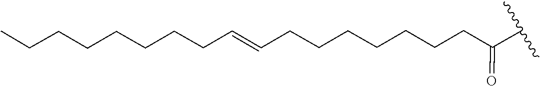 [SMILES: *C(=O)CCCCCCC/C=C/CCCCCCCC] | 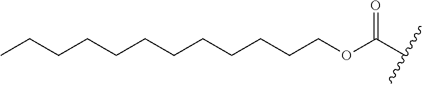 [SMILES: *C(=O)OCCCCCCCCCCCC] |
| 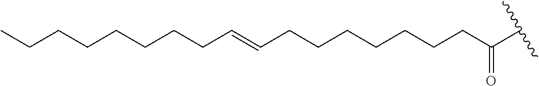 [SMILES: *C(=O)CCCCCCC/C=C/CCCCCCCC] | 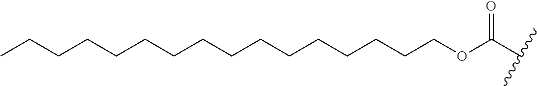 [SMILES: *C(=O)OCCCCCCCCCCCCCCCC] |
| 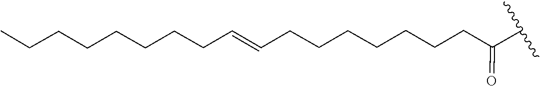 [SMILES: *C(=O)CCCCCCC/C=C/CCCCCCCC] | 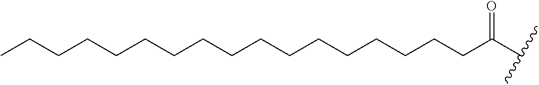 [SMILES: *C(=O)CCCCCCCCCCCCCCCCC] |
| 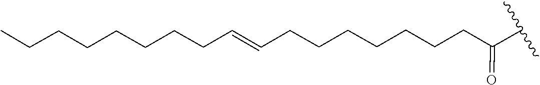 [SMILES: *C(=O)CCCCCCC/C=C/CCCCCCCC] | 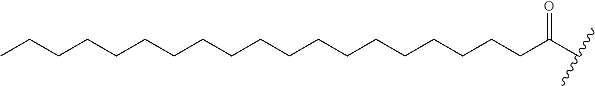 [SMILES: *C(=O)CCCCCCCCCCCCCCCCCCC] |
| 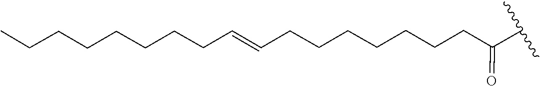 [SMILES: *C(=O)CCCCCCC/C=C/CCCCCCCC] | 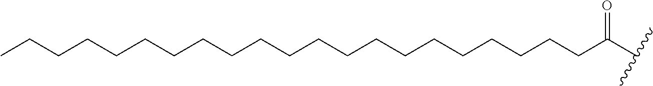 [SMILES: *C(=O)CCCCCCCCCCCCCCCCCCCCC] |
| 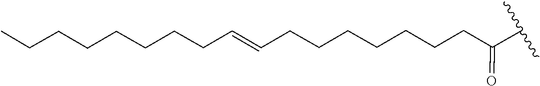 [SMILES: *C(=O)CCCCCCC/C=C/CCCCCCCC] | 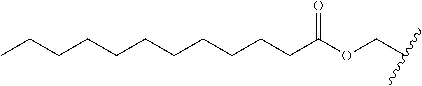 [SMILES: *COC(=O)CCCCCCCCCCC] |
| 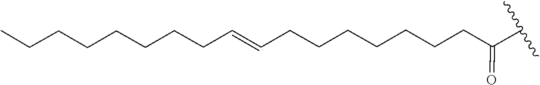 [SMILES: *C(=O)CCCCCCC/C=C/CCCCCCCC] | 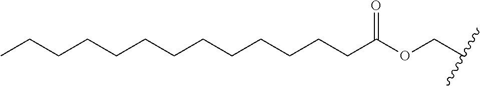 [SMILES: *COC(=O)CCCCCCCCCCCCC] |
| 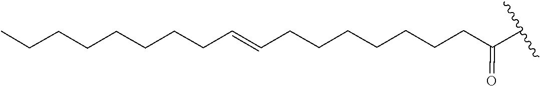 [SMILES: *C(=O)CCCCCCC/C=C/CCCCCCCC] | 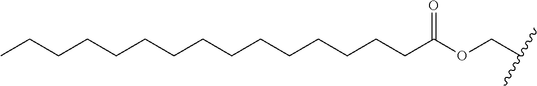 [SMILES: *COC(=O)CCCCCCCCCCCCCCC] |
| 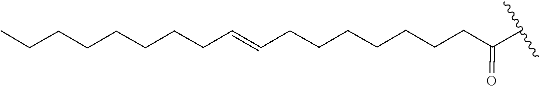 [SMILES: *C(=O)CCCCCCC/C=C/CCCCCCCC] | 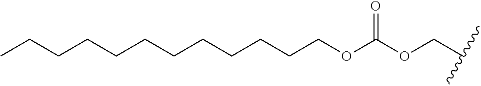 [SMILES: *COC(=O)OCCCCCCCCCCCC] |
| 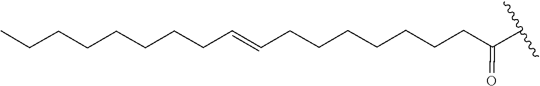 [SMILES: *C(=O)CCCCCCC/C=C/CCCCCCCC] | 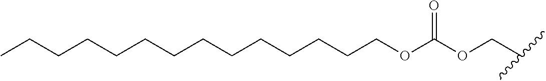 [SMILES: *COC(=O)OCCCCCCCCCCCCCC] |
| 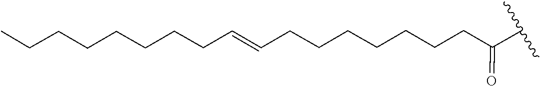 [SMILES: *C(=O)CCCCCCC/C=C/CCCCCCCC] | 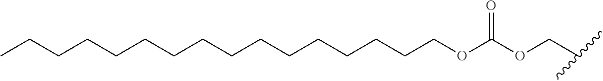 [SMILES: *COC(=O)OCCCCCCCCCCCCCCCC] |
| 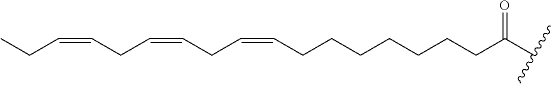 [SMILES: *C(=O)CCCCCCC/C=C\C/C=C\C/C=C\CC] | 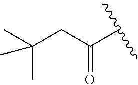 [SMILES: *C(=O)CC(C)(C)C] |
| 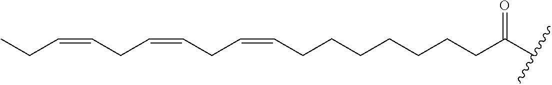 [SMILES: *C(=O)CCCCCCC/C=C\C/C=C\C/C=C\CC] | 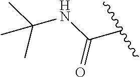 [SMILES: *C(=O)NC(C)(C)C] |
| 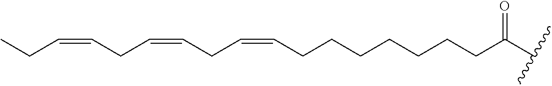 [SMILES: *C(=O)CCCCCCC/C=C\C/C=C\C/C=C\CC] | 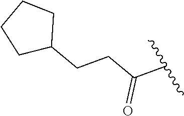 [SMILES: *C(=O)CCC1CCCC1] |
| 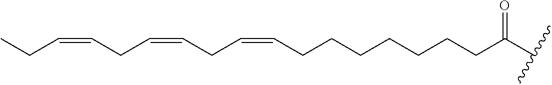 [SMILES: *C(=O)CCCCCCC/C=C\C/C=C\C/C=C\CC] | 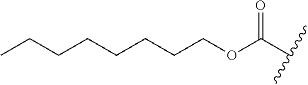 [SMILES: *C(=O)OCCCCCCCC] |
| 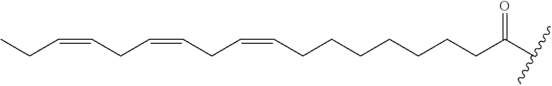 [SMILES: *C(=O)CCCCCCC/C=C\C/C=C\C/C=C\CC] | 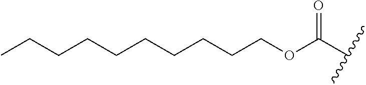 [SMILES: *C(=O)OCCCCCCCCCC] |
| 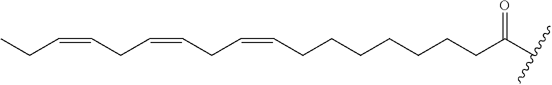 [SMILES: *C(=O)CCCCCCC/C=C\C/C=C\C/C=C\CC] | 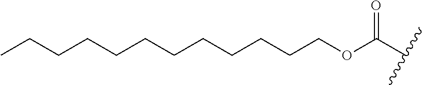 [SMILES: *C(=O)OCCCCCCCCCCCC] |
| 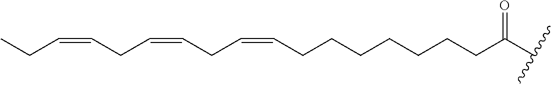 [SMILES: *C(=O)CCCCCCC/C=C\C/C=C\C/C=C\CC] | 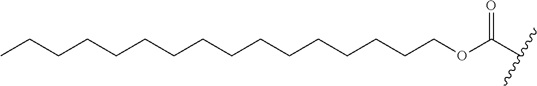 [SMILES: *C(=O)OCCCCCCCCCCCCCCCC] |
| 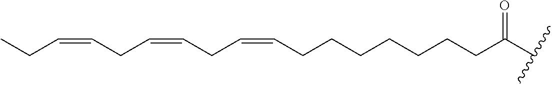 [SMILES: *C(=O)CCCCCCC/C=C\C/C=C\C/C=C\CC] | 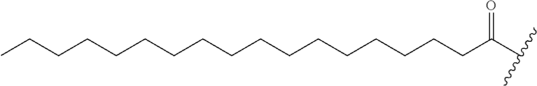 [SMILES: *C(=O)CCCCCCCCCCCCCCCCC] |
| 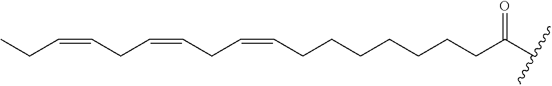 [SMILES: *C(=O)CCCCCCC/C=C\C/C=C\C/C=C\CC] | 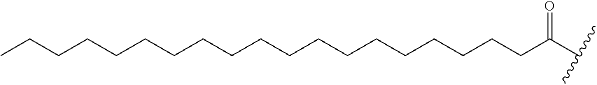 [SMILES: *C(=O)CCCCCCCCCCCCCCCCCCC] |
| 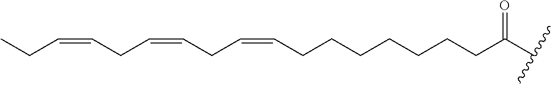 [SMILES: *C(=O)CCCCCCC/C=C\C/C=C\C/C=C\CC] | 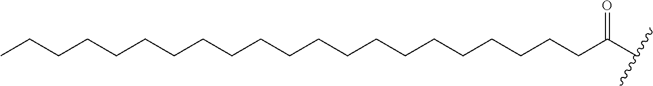 [SMILES: *C(=O)CCCCCCCCCCCCCCCCCCCCC] |
| 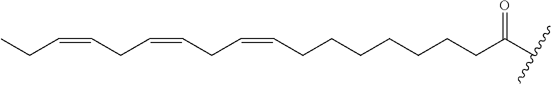 [SMILES: *C(=O)CCCCCCC/C=C\C/C=C\C/C=C\CC] | 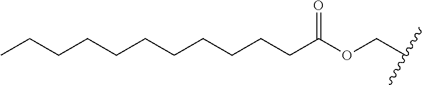 [SMILES: *COC(=O)CCCCCCCCCCC] |
| 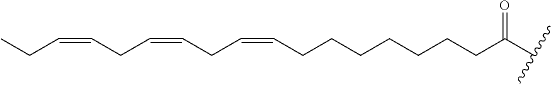 [SMILES: *C(=O)CCCCCCC/C=C\C/C=C\C/C=C\CC] | 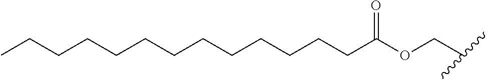 [SMILES: *COC(=O)CCCCCCCCCCCCC] |
| 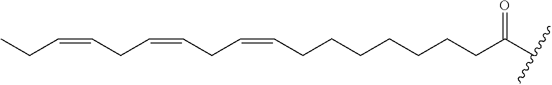 [SMILES: *C(=O)CCCCCCC/C=C\C/C=C\C/C=C\CC] | 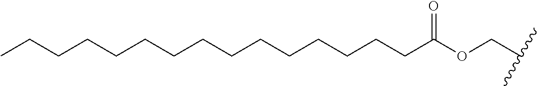 [SMILES: *COC(=O)CCCCCCCCCCCCCCC] |
| 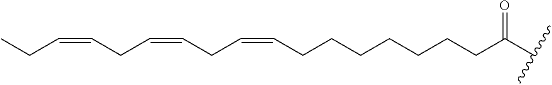 [SMILES: *C(=O)CCCCCCC/C=C\C/C=C\C/C=C\CC] | 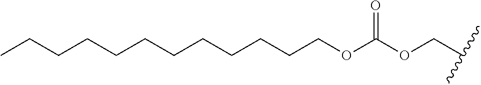 [SMILES: *COC(=O)OCCCCCCCCCCCC] |
| 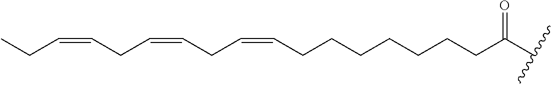 [SMILES: *C(=O)CCCCCCC/C=C\C/C=C\C/C=C\CC] | 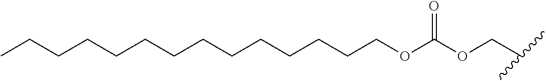 [SMILES: *COC(=O)OCCCCCCCCCCCCCC] |
| 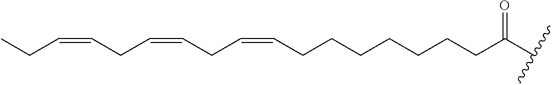 [SMILES: *C(=O)CCCCCCC/C=C\C/C=C\C/C=C\CC] | 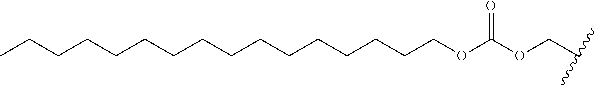 [SMILES: *COC(=O)OCCCCCCCCCCCCCCCC] |
| tBu | 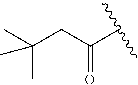 [SMILES: *C(=O)CC(C)(C)C] |
| tBu | 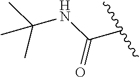 [SMILES: *C(=O)NC(C)(C)C] |
| tBu | 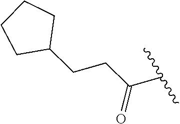 [SMILES: *C(=O)CCC1CCCC1] |
| tBu | 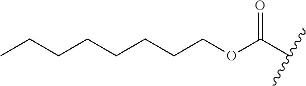 [SMILES: *C(=O)OCCCCCCCC] |
| tBu | 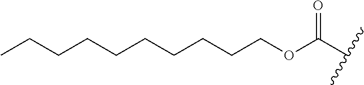 [SMILES: *C(=O)OCCCCCCCCCC] |
| tBu | 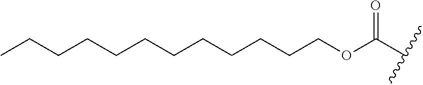 [SMILES: *C(=O)OCCCCCCCCCCCC] |
| tBu | 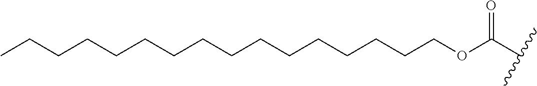 [SMILES: *C(=O)OCCCCCCCCCCCCCCCC] |
| tBu | 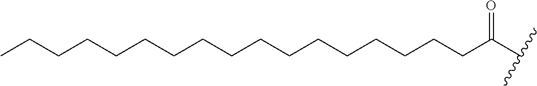 [SMILES: *C(=O)CCCCCCCCCCCCCCCCC] |
| tBu | 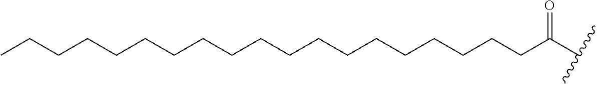 [SMILES: *C(=O)CCCCCCCCCCCCCCCCCCC] |
| tBu | 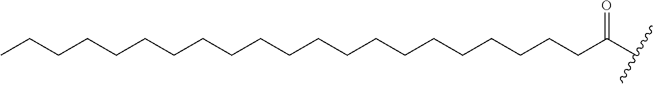 [SMILES: *C(=O)CCCCCCCCCCCCCCCCCCCCC] |
| tBu | 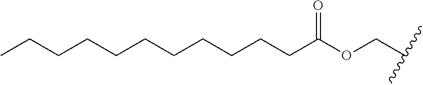 [SMILES: *COC(=O)CCCCCCCCCCC] |
| tBu | 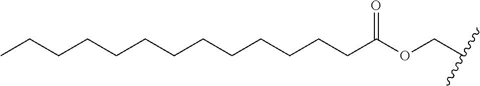 [SMILES: *COC(=O)CCCCCCCCCCCCC] |
| tBu | 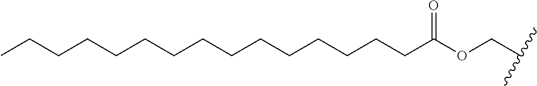 [SMILES: *COC(=O)CCCCCCCCCCCCCCC] |
| tBu | 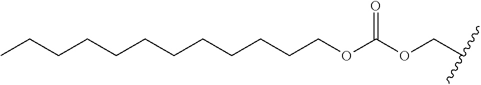 [SMILES: *COC(=O)OCCCCCCCCCCCC] |
| tBu | 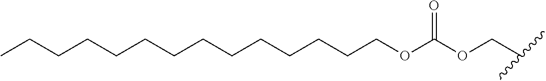 [SMILES: *COC(=O)OCCCCCCCCCCCCCC] |
| tBu | 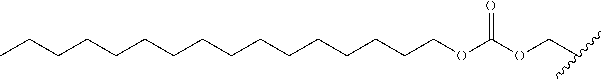 [SMILES: *COC(=O)OCCCCCCCCCCCCCCCC] |
| phenyl | 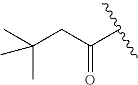 [SMILES: *C(=O)CC(C)(C)C] |
| phenyl | 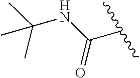 [SMILES: *C(=O)NC(C)(C)C] |
| phenyl | 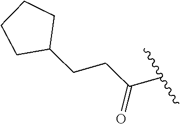 [SMILES: *C(=O)CCC1CCCC1] |
| phenyl | 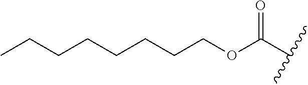 [SMILES: *C(=O)OCCCCCCCC] |
| phenyl | 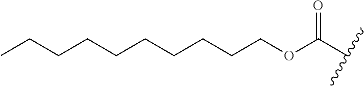 [SMILES: *C(=O)OCCCCCCCCCC] |
| phenyl | 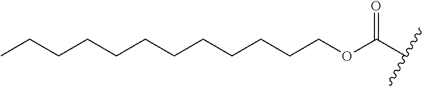 [SMILES: *C(=O)OCCCCCCCCCCCC] |
| phenyl | 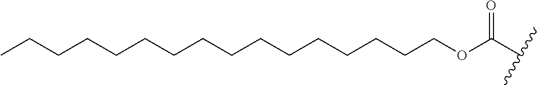 [SMILES: *C(=O)OCCCCCCCCCCCCCCCC] |
| phenyl | 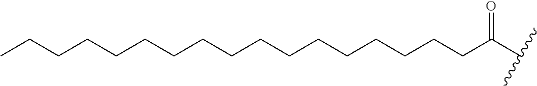 [SMILES: *C(=O)CCCCCCCCCCCCCCCCC] |
| phenyl | 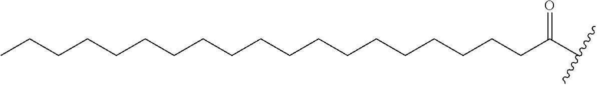 [SMILES: *C(=O)CCCCCCCCCCCCCCCCCCC] |
| phenyl | 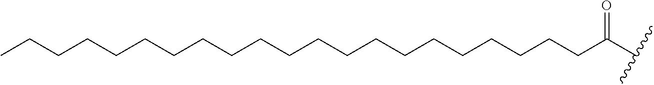 [SMILES: *C(=O)CCCCCCCCCCCCCCCCCCCCC] |
| phenyl | 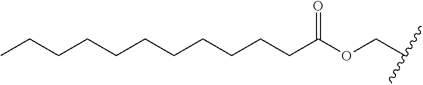 [SMILES: *COC(=O)CCCCCCCCCCC] |
| phenyl | 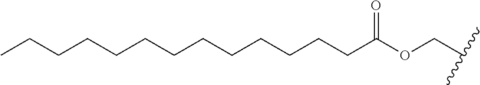 [SMILES: *COC(=O)CCCCCCCCCCCCC] |
| phenyl | 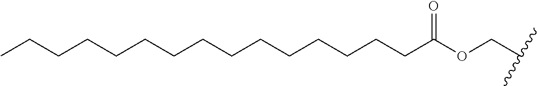 [SMILES: *COC(=O)CCCCCCCCCCCCCCC] |
| phenyl | 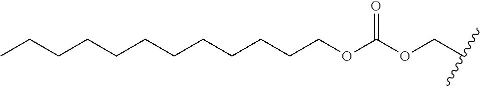 [SMILES: *COC(=O)OCCCCCCCCCCCC] |
| phenyl | 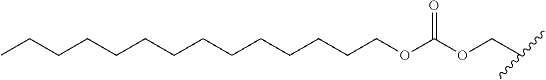 [SMILES: *COC(=O)OCCCCCCCCCCCCCC] |
| phenyl | 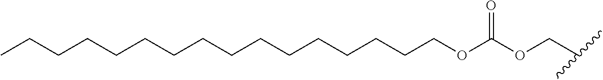 [SMILES: *COC(=O)OCCCCCCCCCCCCCCCC] |
| 4-chlorophenyl | 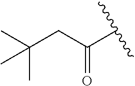 [SMILES: *C(=O)CC(C)(C)C] |
| 4-chlorophenyl | 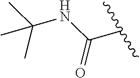 [SMILES: *C(=O)NC(C)(C)C] |
| 4-chlorophenyl | 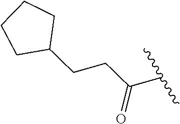 [SMILES: *C(=O)CCC1CCCC1] |
| 4-chlorophenyl | 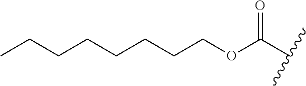 [SMILES: *C(=O)OCCCCCCCC] |
| 4-chlorophenyl | 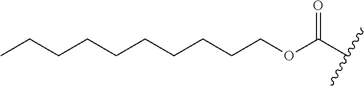 [SMILES: *C(=O)OCCCCCCCCCC] |
| 4-chlorophenyl | 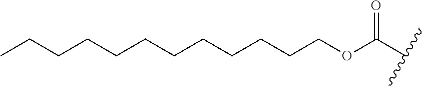 [SMILES: *C(=O)OCCCCCCCCCCCC] |
| 4-chlorophenyl | 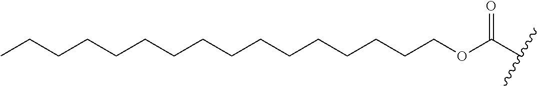 [SMILES: *C(=O)OCCCCCCCCCCCCCCCC] |
| 4-chlorophenyl | 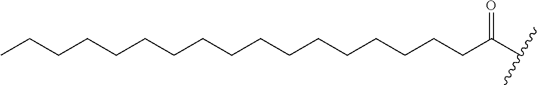 [SMILES: *C(=O)CCCCCCCCCCCCCCCCC] |
| 4-chlorophenyl | 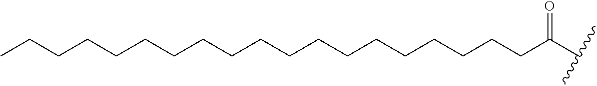 [SMILES: *C(=O)CCCCCCCCCCCCCCCCCCC] |
| 4-chlorophenyl | 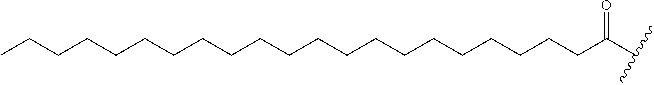 [SMILES: *C(=O)CCCCCCCCCCCCCCCCCCCCC] |
| 4-chlorophenyl | 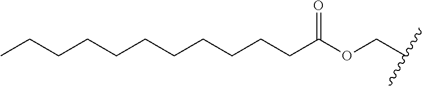 [SMILES: *COC(=O)CCCCCCCCCCC] |
| 4-chlorophenyl | 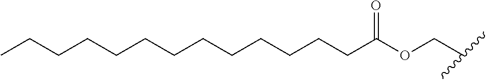 [SMILES: *COC(=O)CCCCCCCCCCCCC] |
| 4-chlorophenyl | 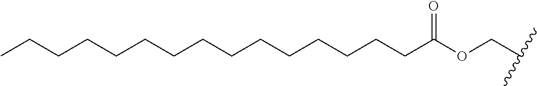 [SMILES: *COC(=O)CCCCCCCCCCCCCCC] |
| 4-chlorophenyl | 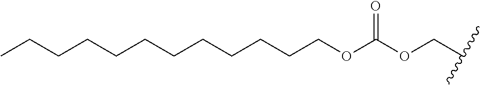 [SMILES: *COC(=O)OCCCCCCCCCCCC] |
| 4-chlorophenyl | 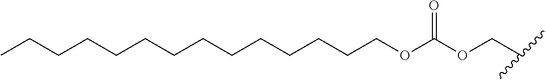 [SMILES: *COC(=O)OCCCCCCCCCCCCCC] |
| 4-chlorophenyl | 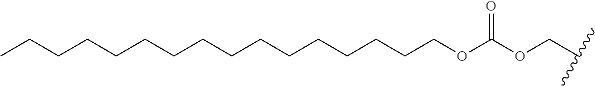 [SMILES: *COC(=O)OCCCCCCCCCCCCCCCC] |
| 3,4-dichlorophenyl | 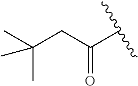 [SMILES: *C(=O)CC(C)(C)C] |
| 3,4-dichlorophenyl | 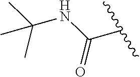 [SMILES: *C(=O)NC(C)(C)C] |
| 3,4-dichlorophenyl | 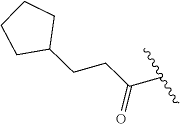 [SMILES: *C(=O)CCC1CCCC1] |
| 3,4-dichlorophenyl | 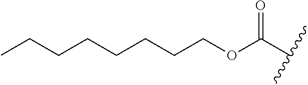 [SMILES: *C(=O)OCCCCCCCC] |
| 3,4-dichlorophenyl | 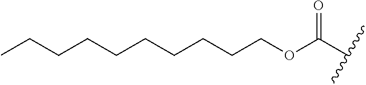 [SMILES: *C(=O)OCCCCCCCCCC] |
| 3,4-dichlorophenyl | 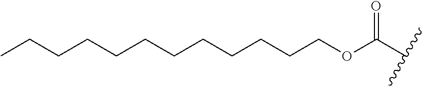 [SMILES: *C(=O)OCCCCCCCCCCCC] |
| 3,4-dichlorophenyl | 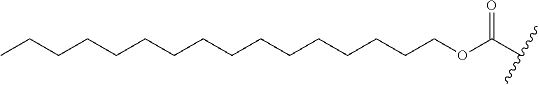 [SMILES: *C(=O)OCCCCCCCCCCCCCCCC] |
| 3,4-dichlorophenyl | 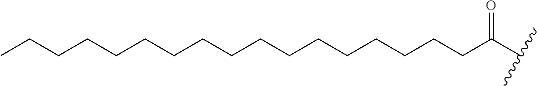 [SMILES: *C(=O)CCCCCCCCCCCCCCCCC] |
| 3,4-dichlorophenyl | 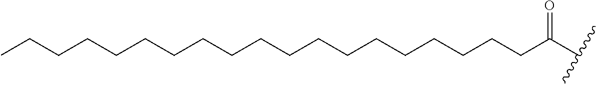 [SMILES: *C(=O)CCCCCCCCCCCCCCCCCCC] |
| 3,4-dichlorophenyl | 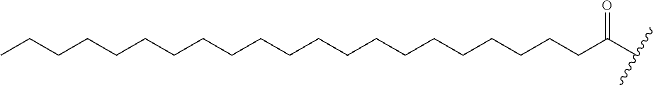 [SMILES: *C(=O)CCCCCCCCCCCCCCCCCCCCC] |
| 3,4-dichlorophenyl | 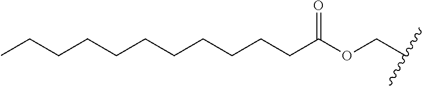 [SMILES: *COC(=O)CCCCCCCCCCC] |
| 3,4-dichlorophenyl | 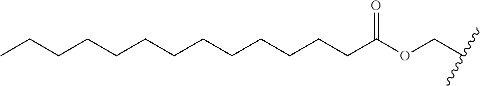 [SMILES: *COC(=O)CCCCCCCCCCCCC] |
| 3,4-dichlorophenyl | 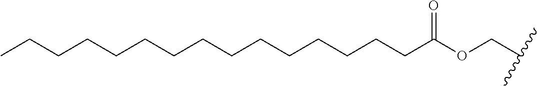 [SMILES: *COC(=O)CCCCCCCCCCCCCCC] |
| 3,4-dichlorophenyl | 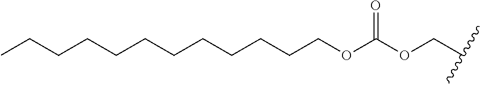 [SMILES: *COC(=O)OCCCCCCCCCCCC] |
| 3,4-dichlorophenyl | 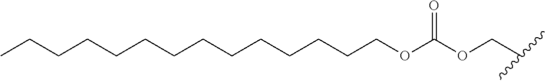 [SMILES: *COC(=O)OCCCCCCCCCCCCCC] |
| 3,4-dichlorophenyl | 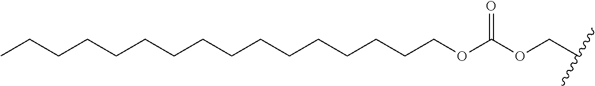 [SMILES: *COC(=O)OCCCCCCCCCCCCCCCC] |
| 4-methylphenyl | 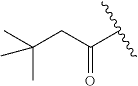 [SMILES: *C(=O)CC(C)(C)C] |
| 4-methylphenyl | 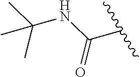 [SMILES: *C(=O)NC(C)(C)C] |
| 4-methylphenyl | 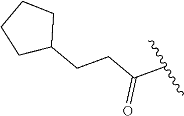 [SMILES: *C(=O)CCC1CCCC1] |
| 4-methylphenyl | 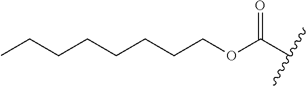 [SMILES: *C(=O)OCCCCCCCC] |
| 4-methylphenyl | 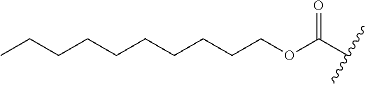 [SMILES: *C(=O)OCCCCCCCCCC] |
| 4-methylphenyl | 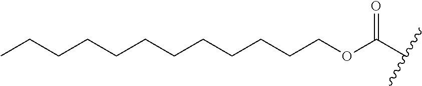 [SMILES: *C(=O)OCCCCCCCCCCCC] |
| 4-methylphenyl | 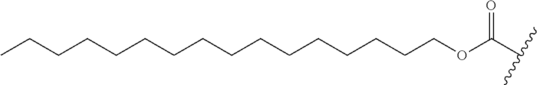 [SMILES: *C(=O)OCCCCCCCCCCCCCCCC] |
| 4-methylphenyl | 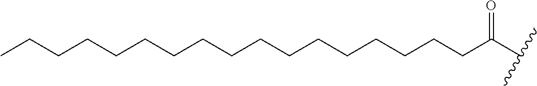 [SMILES: *C(=O)CCCCCCCCCCCCCCCCC] |
| 4-methylphenyl | 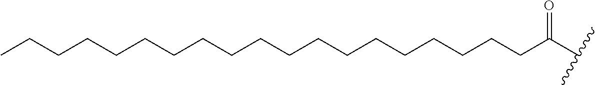 [SMILES: *C(=O)CCCCCCCCCCCCCCCCCCC] |
| 4-methylphenyl | 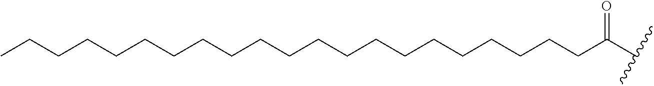 [SMILES: *C(=O)CCCCCCCCCCCCCCCCCCCCC] |
| 4-methylphenyl | 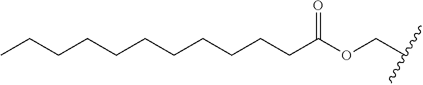 [SMILES: *COC(=O)CCCCCCCCCCC] |
| 4-methylphenyl | 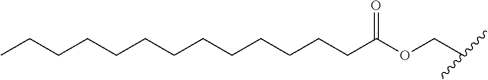 [SMILES: *COC(=O)CCCCCCCCCCCCC] |
| 4-methylphenyl | 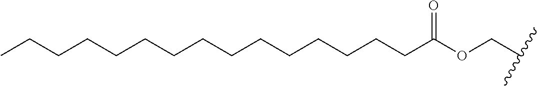 [SMILES: *COC(=O)CCCCCCCCCCCCCCC] |
| 4-methylphenyl | 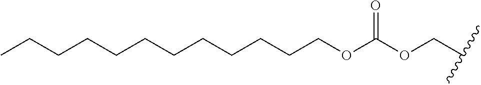 [SMILES: *COC(=O)OCCCCCCCCCCCC] |
| 4-methylphenyl | 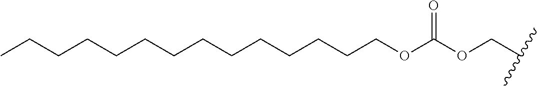 [SMILES: *COC(=O)OCCCCCCCCCCCCCC] |
| 4-methylphenyl | 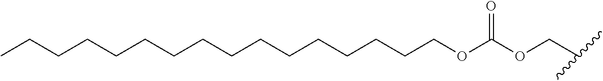 [SMILES: *COC(=O)OCCCCCCCCCCCCCCCC] |
| 4-methoxyphenyl | 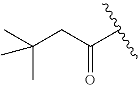 [SMILES: *C(=O)CC(C)(C)C] |
| 4-methoxyphenyl | 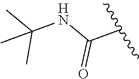 [SMILES: *C(=O)NC(C)(C)C] |
| 4-methoxyphenyl | 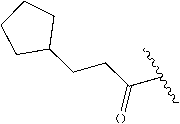 [SMILES: *C(=O)CCC1CCCC1] |
| 4-methoxyphenyl | 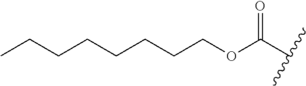 [SMILES: *C(=O)OCCCCCCCC] |
| 4-methoxyphenyl | 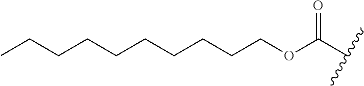 [SMILES: *C(=O)OCCCCCCCCCC] |
| 4-methoxyphenyl | 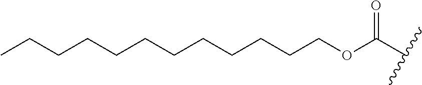 [SMILES: *C(=O)OCCCCCCCCCCCC] |
| 4-methoxyphenyl | 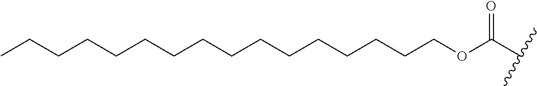 [SMILES: *C(=O)OCCCCCCCCCCCCCCCC] |
| 4-methoxyphenyl | 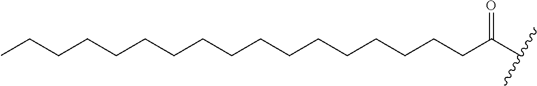 [SMILES: *C(=O)CCCCCCCCCCCCCCCCC] |
| 4-methoxyphenyl | 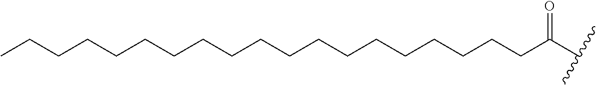 [SMILES: *C(=O)CCCCCCCCCCCCCCCCCCC] |
| 4-methoxyphenyl | 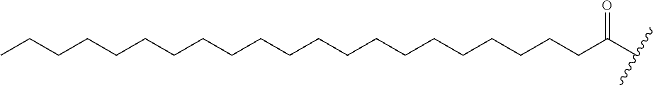 [SMILES: *C(=O)CCCCCCCCCCCCCCCCCCCCC] |
| 4-methoxyphenyl | 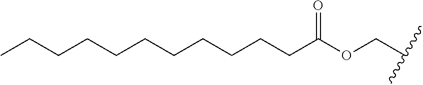 [SMILES: *COC(=O)CCCCCCCCCCC] |
| 4-methoxyphenyl | 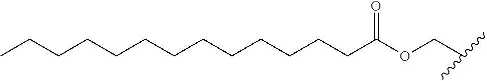 [SMILES: *COC(=O)CCCCCCCCCCCCC] |
| 4-methoxyphenyl | 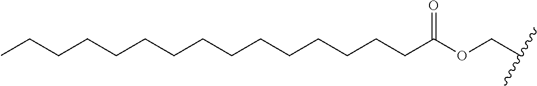 [SMILES: *COC(=O)CCCCCCCCCCCCCCC] |
| 4-methoxyphenyl | 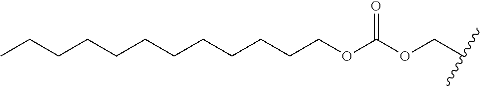 [SMILES: *COC(=O)OCCCCCCCCCCCC] |
| 4-methoxyphenyl | 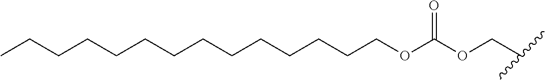 [SMILES: *COC(=O)OCCCCCCCCCCCCCC] |
| 4-methoxyphenyl | 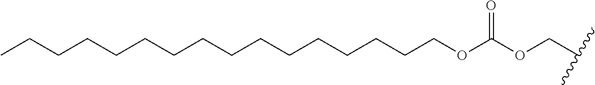 [SMILES: *COC(=O)OCCCCCCCCCCCCCCCC] |

In another embodiment, the invention is a compound represented by a structure selected from:

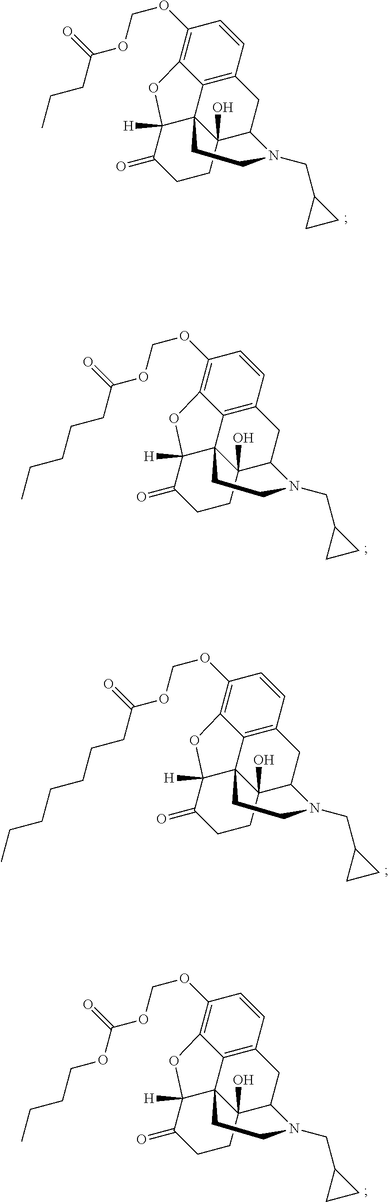

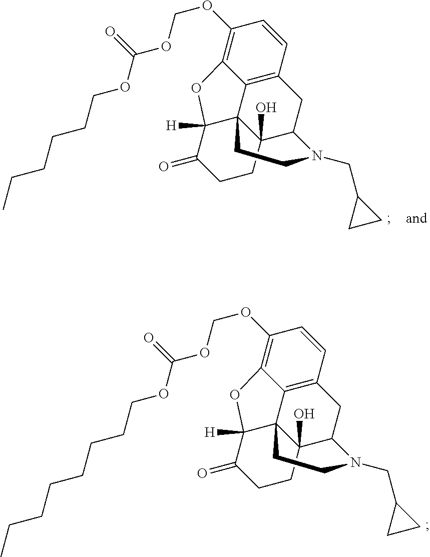

or a pharmaceutically acceptable salt thereof.

In another embodiment, the invention is a compound represented by a structure selected from:

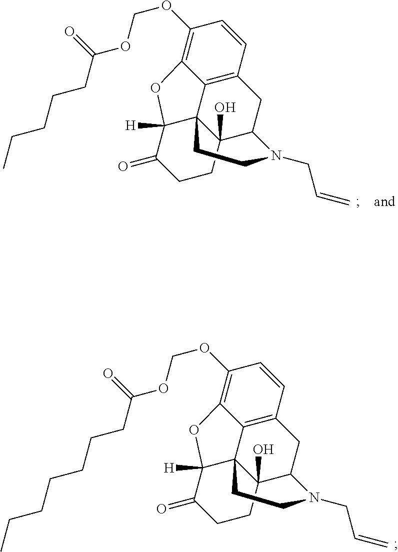

or a pharmaceutically acceptable salt thereof.

In a 67^{th} aspect, provided herein is a compound, or pharmaceutically acceptable salt thereof, having a structure provided in Formula (IV-1),

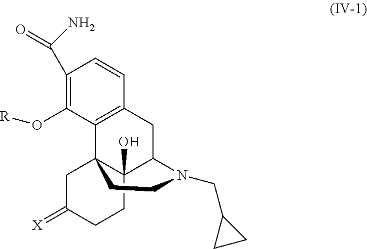

wherein:
X is O or CH₂;
R is R¹OCH₂;
wherein R¹ is —(CH₂)_{q}CH₃; and
wherein q is an integer between 0 and 25.

In one specific embodiment, q is 0 and X is; in another specific embodiment, q is 0 and X is CH₂; in yet another specific embodiment, q is 1 and X is 0; in yet another specific embodiment, q is 1 and X is CH₂; in yet another specific embodiment, q is 2 and X is 0; in yet another specific embodiment, q is 2 and X is CH₂; in yet another specific embodiment, q is 3 and X is 0; in yet another specific embodiment, q is 3 and X is CH₂; in yet another specific embodiment, q is 4 and X is 0; in yet another specific embodiment, q is 4 and X is CH₂; in yet another specific embodiment, q is 5 and X is 0; in yet another specific embodiment, q is 5 and X is CH₂; in yet another specific embodiment, q is 6 and X is 0; in yet another specific embodiment, q is 6 and X is CH₂; in yet another specific embodiment, q is 7 and X is 0; in yet another specific embodiment, q is 7 and X is CH₂; in yet another specific embodiment, q is 8 and X is 0; in yet another specific embodiment, q is 8 and X is CH₂; in yet another specific embodiment, q is 9 and X is 0; in yet another specific embodiment, q is 9 and X is CH₂; in yet another specific embodiment, q is 10 and X is 0; in yet another specific embodiment, q is 10 and X is CH₂; in yet another specific embodiment, q is 11 and X is O; in yet another specific embodiment, q is 11 and X is CH₂; in yet another specific embodiment, q is 12 and X is 0; in yet another specific embodiment, q is 12 and X is CH₂; in yet another specific embodiment, q is 13 and X is 0; in yet another specific embodiment, q is 13 and X is CH₂; in yet another specific embodiment, q is 14 and X is 0; in yet another specific embodiment, q is 14 and X is CH₂; in yet another specific embodiment, q is 15 and X is 0; in yet another specific embodiment, q is 15 and X is CH₂; in yet another specific embodiment, q is 16 and X is 0; in yet another specific embodiment, q is 16 and X is CH₂; in yet another specific embodiment, q is 17 and X is 0; in yet another specific embodiment, q is 17 and X is CH₂; in yet another specific embodiment, q is 18 and X is O; in yet another specific embodiment, q is 18 and X is CH₂; in yet another specific embodiment, q is 19 and X is 0; in yet another specific embodiment, q is 19 and X is CH₂; in yet another specific embodiment, q is 20 and X is 0; in yet another specific embodiment, q is 20 and X is CH₂; in yet another specific embodiment, q is 21 and X is 0; in yet another specific embodiment, q is 21 and X is CH₂; in yet another specific embodiment, q is 22 and X is 0; in yet another specific embodiment, q is 22 and X is CH₂; in yet another specific embodiment, q is 23 and X is 0; in yet another specific embodiment, q is 23 and X is CH₂; in yet another specific embodiment, q is 24 and X is 0; in yet another specific embodiment, q is 24 and X is CH₂; in yet another specific embodiment, q is 25 and X is O; and in yet another specific embodiment, q is 25 and X is CH₂. In all of the specific embodiments described in this paragraph, R and R¹ are as described above for Formula (IV-1).

Pharmaceutically acceptable salts of any one of the foregoing embodiments described in the 67^{th} aspect are also included.

In a 68^{th} aspect, provided herein is a compound, or pharmaceutically acceptable salt thereof, having a structure provided in Formula (IV-2),

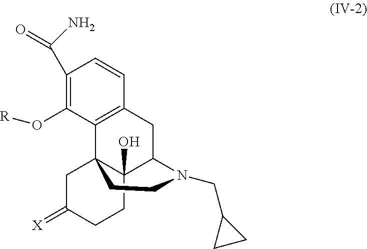

wherein:
X is O or CH₂;
R is R¹OCH₂;
wherein R¹ is

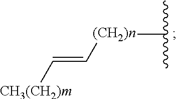

and
wherein m and n are integers between 0 and 23, and the sum of m and n is an integer between 6 and 23.

In another specific aspect, X, m and n in Formula (IV-2) are as described in the First Specific Embodiment for Formula (I-5); and R and R¹ are as described above for Formula (IV-2).

In another specific aspect, X, m and n in Formula (IV-2) are as described in the Second Specific Embodiment for Formula (I-5); and R and R¹ are as described above for Formula (IV-2).

In another specific aspect, X, m and n in Formula (IV-2) are as described in the Third Specific Embodiment for Formula (I-5); and R and R¹ are as described above for Formula (IV-2).

In another specific aspect, X, m and n in Formula (IV-2) are as described in the Fourth Specific Embodiment for Formula (I-5); and R and R¹ are as described above for Formula (IV-2).

In another specific aspect, X, m and n in Formula (IV-2) are as described in the Fifth Specific Embodiment for Formula (I-5); and R and R¹ are as described above for Formula (IV-2).

In another specific aspect, X, m and n in Formula (IV-2) are as described in the Sixth Specific Embodiment for Formula (I-5); and R and R¹ are as described above for Formula (IV-2).

Pharmaceutically acceptable salts of any one of the foregoing specific aspects for the 68^{th} aspect are also included.

In a 69^{th} aspect, provided herein is a compound, or pharmaceutically acceptable salt thereof, having a structure provided in Formula (IV-3),

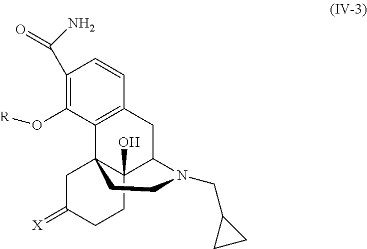

wherein:
X is O or CH₂;
R is R¹OCH₂;
wherein R¹ is

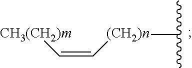

and
wherein m and n are integers between 0 and 23, and the sum of m and n is an integer between 6 and 23.

In another specific aspect, X, m and n in Formula (IV-3) are as described in the First Specific Embodiment for Formula (I-5); and R and R¹ are as described above for Formula (IV-3).

In another specific aspect, X, m and n in Formula (IV-3) are as described in the Second Specific Embodiment for Formula (I-5); and R and R¹ are as described above for Formula (IV-3).

In another specific aspect, X, m and n in Formula (IV-3) are as described in the Third Specific Embodiment for Formula (I-5); and R and R¹ are as described above for Formula (IV-3).

In another specific aspect, X, m and n in Formula (IV-3) are as described in the Fourth Specific Embodiment for Formula (I-5); and R and R¹ are as described above for Formula (IV-3).

In another specific aspect, X, m and n in Formula (IV-3) are as described in the Fifth Specific Embodiment for Formula (I-5); and R and R¹ are as described above for Formula (IV-3).

In another specific aspect, X, m and n in Formula (IV-3) are as described in the Sixth Specific Embodiment for Formula (I-5); and R and R¹ are as described above for Formula (IV-3).

Pharmaceutically acceptable salts of any one of the foregoing specific aspects for the 69^{th} aspect are also included.

In a 70^{th} aspect, provided herein is a compound, or pharmaceutically acceptable salt thereof, having a structure provided in Formula (V-1),

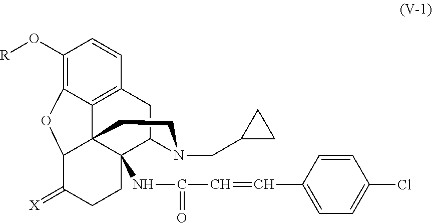

wherein:
X is O or CH₂;
R is R¹OCH₂;
wherein R¹ is —(CH₂)_{q}CH₃; and
wherein q is an integer between 0 and 25.

In one specific embodiment, q is 0 and X is O; in another specific embodiment, q is 0 and X is CH₂; in yet another specific embodiment, q is 1 and X is O; in yet another specific embodiment, q is 1 and X is CH₂; in yet another specific embodiment, q is 2 and X is O; in yet another specific embodiment, q is 2 and X is CH₂; in yet another specific embodiment, q is 3 and X is O; in yet another specific embodiment, q is 3 and X is CH₂; in yet another specific embodiment, q is 4 and X is O; in yet another specific embodiment, q is 4 and X is CH₂; in yet another specific embodiment, q is 5 and X is O; in yet another specific embodiment, q is 5 and X is CH₂; in yet another specific embodiment, q is 6 and X is O; in yet another specific embodiment, q is 6 and X is CH₂; in yet another specific embodiment, q is 7 and X is O; in yet another specific embodiment, q is 7 and X is CH₂; in yet another specific embodiment, q is 8 and X is O; in yet another specific embodiment, q is 8 and X is CH₂; in yet another specific embodiment, q is 9 and X is O; in yet another specific embodiment, q is 9 and X is CH₂; in yet another specific embodiment, q is 10 and X is O; in yet another specific embodiment, q is 10 and X is CH₂; in yet another specific embodiment, q is 11 and X is O; in yet another specific embodiment, q is 11 and X is CH₂; in yet another specific embodiment, q is 12 and X is O; in yet another specific embodiment, q is 12 and X is CH₂; in yet another specific embodiment, q is 13 and X is O; in yet another specific embodiment, q is 13 and X is CH₂; in yet another specific embodiment, q is 14 and X is O; in yet another specific embodiment, q is 14 and X is CH₂; in yet another specific embodiment, q is 15 and X is O; in yet another specific embodiment, q is 15 and X is CH₂; in yet another specific embodiment, q is 16 and X is O; in yet another specific embodiment, q is 16 and X is CH₂; in yet another specific embodiment, q is 17 and X is O; in yet another specific embodiment, q is 17 and X is CH₂; in yet another specific embodiment, q is 18 and X is O; in yet another specific embodiment, q is 18 and X is CH₂; in yet another specific embodiment, q is 19 and X is O; in yet another specific embodiment, q is 19 and X is CH₂; in yet another specific embodiment, q is 20 and X is O; in yet another specific embodiment, q is 20 and X is CH₂; in yet another specific embodiment, q is 21 and X is O; in yet another specific embodiment, q is 21 and X is CH₂; in yet another specific embodiment, q is 22 and X is O; in yet another specific embodiment, q is 22 and X is CH₂; in yet another specific embodiment, q is 23 and X is O; in yet another specific embodiment, q is 23 and X is CH₂; in yet another specific embodiment, q is 24 and X is O; in yet another specific embodiment, q is 24 and X is CH₂; in yet another specific embodiment, q is 25 and X is O; and in yet another specific embodiment, q is 25 and X is CH₂. In all of the specific aspects described in this paragraph, R and R¹ are as described above for Formula (V-1).

Pharmaceutically acceptable salts of any one of the foregoing embodiments described in the 70^{th} aspect are also included.

In a 71^{th} aspect, provided herein is a compound, or pharmaceutically acceptable salt thereof, having a structure provided in Formula (V-2),

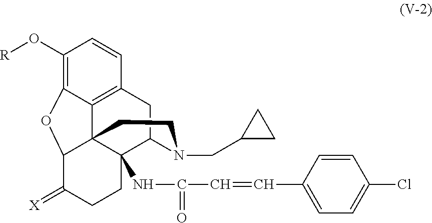

wherein:
X is O or CH₂;
R is R¹OCH₂;
wherein R¹ is

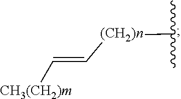

and
wherein m and n are integers between 0 and 23, and the sum of m and n is an integer between 6 and 23.

In another specific aspect, X, m and n in Formula (V-2) are as described in the First Specific Embodiment for Formula (I-5); and R and R¹ are as described above for Formula (V-2).

In another specific aspect, X, m and n in Formula (V-2) are as described in the Second Specific Embodiment for Formula (I-5); and R and R¹ are as described above for Formula (V-2).

In another specific aspect, X, m and n in Formula (V-2) are as described in the Third Specific Embodiment for Formula (I-5); and R and R¹ are as described above for Formula (V-2).

In another specific aspect, X, m and n in Formula (V-2) are as described in the Fourth Specific Embodiment for Formula (I-5); and R and R¹ are as described above for Formula (V-2).

In another specific aspect, X, m and n in Formula (V-2) are as described in the Fifth Specific Embodiment for Formula (I-5); and R and R¹ are as described above for Formula (V-2).

In another specific aspect, X, m and n in Formula (V-2) are as described in the Sixth Specific Embodiment for Formula (I-5); and R and R¹ are as described above for Formula (V-2).

Pharmaceutically acceptable salts of any one of the foregoing specific aspects for the 71^{th} aspect are also included.

In a 72^{th} aspect, provided herein is a compound, or pharmaceutically acceptable salt thereof, having a structure provided in Formula (V-3),

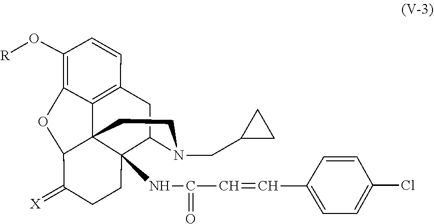

wherein:
X is O or CH₂;
R is R¹OCH₂;
wherein R¹ is

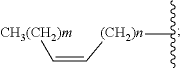

and
wherein m and n are integers between 0 and 23, and the sum of m and n is an integer between 6 and 23.

In another specific aspect, X, m and n in Formula (V-3) are as described in the First Specific Embodiment for Formula (I-5); and R and R¹ are as described above for Formula (V-3).

In another specific aspect, X, m and n in Formula (V-3) are as described in the Second Specific Embodiment for Formula (I-5); and R and R¹ are as described above for Formula (V-3).

In another specific aspect, X, m and n in Formula (V-3) are as described in the Third Specific Embodiment for Formula (I-5); and R and R¹ are as described above for Formula (V-3).

In another specific aspect, X, m and n in Formula (V-3) are as described in the Fourth Specific Embodiment for Formula (I-5); and R and R¹ are as described above for Formula (V-3).

In another specific aspect, X, m and n in Formula (V-3) are as described in the Fifth Specific Embodiment for Formula (I-5); and R and R¹ are as described above for Formula (V-3).

In another specific aspect, X, m and n in Formula (V-3) are as described in the Sixth Specific Embodiment for Formula (I-5); and R and R¹ are as described above for Formula (V-3).

Pharmaceutically acceptable salts of any one of the foregoing specific aspects for the 72^{th} aspect are also included.

In a 73^{th} aspect, provided herein is a compound, or pharmaceutically acceptable salt thereof, having a structure provided in Formula (VI-1),

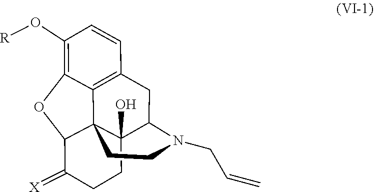

wherein:
X is O or CH₂;
R is R¹OCH₂;
wherein R¹ is —(CH₂)_{q}CH₃; and
wherein q is an integer between 0 and 25.

In one specific embodiment, q is 0 and X is O; in another specific embodiment, q is 0 and X is CH₂; in yet another specific embodiment, q is 1 and X is O; in yet another specific embodiment, q is 1 and X is CH₂; in yet another specific embodiment, q is 2 and X is O; in yet another specific embodiment, q is 2 and X is CH₂; in yet another specific embodiment, q is 3 and X is O; in yet another specific embodiment, q is 3 and X is CH₂; in yet another specific embodiment, q is 4 and X is O; in yet another specific embodiment, q is 4 and X is CH₂; in yet another specific embodiment, q is 5 and X is O; in yet another specific embodiment, q is 5 and X is CH₂; in yet another specific embodiment, q is 6 and X is O; in yet another specific embodiment, q is 6 and X is CH₂; in yet another specific embodiment, q is 7 and X is O; in yet another specific embodiment, q is 7 and X is CH₂; in yet another specific embodiment, q is 8 and X is O; in yet another specific embodiment, q is 8 and X is CH₂; in yet another specific embodiment, q is 9 and X is O; in yet another specific embodiment, q is 9 and X is CH₂; in yet another specific embodiment, q is 10 and X is O; in yet another specific embodiment, q is 10 and X is CH₂; in yet another specific embodiment, q is 11 and X is O; in yet another specific embodiment, q is 11 and X is CH₂; in yet another specific embodiment, q is 12 and X is O; in yet another specific embodiment, q is 12 and X is CH₂; in yet another specific embodiment, q is 13 and X is O; in yet another specific embodiment, q is 13 and X is CH₂; in yet another specific embodiment, q is 14 and X is O; in yet another specific embodiment, q is 14 and X is CH₂; in yet another specific embodiment, q is 15 and X is O; in yet another specific embodiment, q is 15 and X is CH₂; in yet another specific embodiment, q is 16 and X is O; in yet another specific embodiment, q is 16 and X is CH₂; in yet another specific embodiment, q is 17 and X is O; in yet another specific embodiment, q is 17 and X is CH₂; in yet another specific embodiment, q is 18 and X is O; in yet another specific embodiment, q is 18 and X is CH₂; in yet another specific embodiment, q is 19 and X is O; in yet another specific embodiment, q is 19 and X is CH₂; in yet another specific embodiment, q is 20 and X is O; in yet another specific embodiment, q is 20 and X is CH₂; in yet another specific embodiment, q is 21 and X is O; in yet another specific embodiment, q is 21 and X is CH₂; in yet another specific embodiment, q is 22 and X is O; in yet another specific embodiment, q is 22 and X is CH₂; in yet another specific embodiment, q is 23 and X is O; in yet another specific embodiment, q is 23 and X is CH₂; in yet another specific embodiment, q is 24 and X is O; in yet another specific embodiment, q is 24 and X is CH₂; in yet another specific embodiment, q is 25 and X is O; and in yet another specific embodiment, q is 25 and X is CH₂. In all of the specific aspects described in this paragraph, R and R¹ are as described above for Formula (VI-1).

Pharmaceutically acceptable salts of any one of the foregoing embodiments described in the 73^{th} aspect are also included.

In a 74^{th} aspect, provided herein is a compound, or pharmaceutically acceptable salt thereof, having a structure provided in Formula (VI-2),

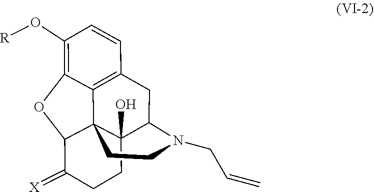

wherein:
X is O or CH₂;
R is R¹OCH₂;
wherein R¹ is

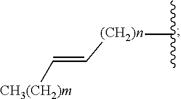

and
wherein m and n are integers between 0 and 23, and the sum of m and n is an integer between 6 and 23.

In another specific aspect, X, m and n in Formula (VI-2) are as described in the First Specific Embodiment for Formula (I-5); and R and R¹ are as described above for Formula (VI-2).

In another specific aspect, X, m and n in Formula (VI-2) are as described in the Second Specific Embodiment for Formula (I-5); and R and R¹ are as described above for Formula (VI-2).

In another specific aspect, X, m and n in Formula (VI-2) are as described in the Third Specific Embodiment for Formula (I-5); and R and R¹ are as described above for Formula (VI-2).

In another specific aspect, X, m and n in Formula (VI-2) are as described in the Fourth Specific Embodiment for Formula (I-5); and R and R¹ are as described above for Formula (VI-2).

In another specific aspect, X, m and n in Formula (VI-2) are as described in the Fifth Specific Embodiment for Formula (I-5); and R and R¹ are as described above for Formula (VI-2).

In another specific aspect, X, m and n in Formula (VI-2) are as described in the Sixth Specific Embodiment for Formula (I-5); and R and R¹ are as described above for Formula (VI-2).

Pharmaceutically acceptable salts of any one of the foregoing specific aspects for the 74^{th} aspect are also included.

In a 75^{th} aspect, provided herein is a compound, or pharmaceutically acceptable salt thereof, having a structure provided in Formula (VI-3),

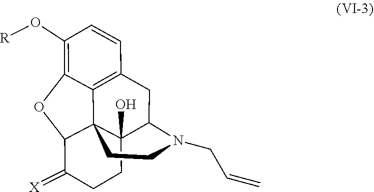

wherein:
X is O or CH₂;
R is R¹OCH₂;
wherein R¹ is

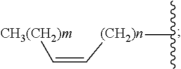

and
wherein m and n are integers between 0 and 23, and the sum of m and n is an integer between 6 and 23.

In another specific aspect, X, m and n in Formula (VI-3) are as described in the First Specific Embodiment for Formula (I-5); and R and R¹ are as described above for Formula (VI-3).

In another specific aspect, X, m and n in Formula (VI-3) are as described in the Second Specific Embodiment for Formula (I-5); and R and R¹ are as described above for Formula (VI-3).

In another specific aspect, X, m and n in Formula (VI-3) are as described in the Third Specific Embodiment for Formula (I-5); and R and R¹ are as described above for Formula (VI-3).

In another specific aspect, X, m and n in Formula (VI-3) are as described in the Fourth Specific Embodiment for Formula (I-5); and R and R¹ are as described above for Formula (VI-3).

In another specific aspect, X, m and n in Formula (VI-3) are as described in the Fifth Specific Embodiment for Formula (I-5); and R and R¹ are as described above for Formula (VI-3).

In another specific aspect, X, m and n in Formula (VI-3) are as described in the Sixth Specific Embodiment for Formula (I-5); and R and R¹ are as described above for Formula (VI-3).

Pharmaceutically acceptable salts of any one of the foregoing specific aspects for the 75^{th} aspect are also included.

These compounds can be used for treating substance abuse, e.g., alcohol or opiate abuse, by administering to a subject in need thereof, an effective or therapeutically effective amount of the compound or a pharmaceutically acceptable amount thereof.

When the configuration of two or more substituents about a double bond is indicated by structure; by “E” or “Z” designations; by “cis” or “trans”; it is to be understood that the depicted geometric isomer purity by weight is at least 85%, at least 90%, at least 95%, at least 97%, at least 98% or at least 99% by weight. Geometric isomer purity by weight means the percent by weight of the compound in a composition having the indicated stereochemistry about the double bond over the total weight of the compound and its corresponding cis/trans geometric isomer.

When the configuration of two or more substituents about a double bond is not indicated by structure or name, it is to be understood that either the “cis” configuration, the “trans” configuration, or any mixture of “cis” or “trans” is intended.

When a particular stereoisomer is indicated by structure or name, it is to be understood that the stereochemical purity is at least 85%, at least 90%, at least 95%, at least 97%, at least 98% or at least 99% by weight. Stereochemical isomer purity by weight means the percent by weight of the stereoisomer in a composition over the total weight of all the corresponding stereoisomers.

Pharmaceutically acceptable salts of the disclosed compounds are included in the invention. “Pharmaceutically acceptable salt” includes both acid and base addition salts. A pharmaceutically acceptable salt of any one of the opioid receptor antagonist prodrug compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

As used herein, “treatment” or “treating,” or “palliating” or “ameliorating” are used interchangeably. These terms refer to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are, in some embodiments, administered to a patient at risk of developing a particular disease, or to a patient reporting one or more of the physiological symptoms of a disease, even though a diagnosis of this disease has not been made.

### Preparation of Compounds

The compounds used in the reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, PA), Aldrich Chemical (Milwaukee, WI, including Sigma Chemical and Fluka), Apin Chemicals Ltd. (Milton Park, UK), Avocado Research (Lancashire, U.K.), BDH Inc. (Toronto, Canada), Bionet (Cornwall, U.K.), Chemservice Inc. (West Chester, PA), Crescent Chemical Co. (Hauppauge, NY), Eastman Organic Chemicals, Eastman Kodak Company (Rochester, NY), Fisher Scientific Co. (Pittsburgh, PA), Fisons Chemicals (Leicestershire, UK), Frontier Scientific (Logan, UT), ICN Biomedicals, Inc. (Costa Mesa, CA), Key Organics (Cornwall, U.K.), Lancaster Synthesis (Windham, NH), Maybridge Chemical Co. Ltd. (Cornwall, U.K.), Parish Chemical Co. (Orem, UT), Pfaltz & Bauer, Inc. (Waterbury, CN), Polyorganix (Houston, TX), Pierce Chemical Co. (Rockford, IL), Riedel de Haen AG (Hanover, Germany), Spectrum Quality Product, Inc. (New Brunswick, NJ), TCI America (Portland, OR), Trans World Chemicals, Inc. (Rockville, MD), and Wako Chemicals USA, Inc. (Richmond, VA).

Suitable reference books and treatise that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatise that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4^{th} Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as through on-line databases (contact the American Chemical Society, Washington, D.C. for more details). Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the opioid receptor antagonist prodrug compounds described herein is P. H. Stahl & C. G. Wermuth “Handbook of Pharmaceutical Salts”, Verlag Helvetica Chimica Acta, Zurich, 2002.

### Pharmaceutical Compositions

In certain embodiments, the opioid receptor antagonist prodrug compound as described herein is administered as a pure chemical. In other embodiments, the opioid receptor antagonist prodrug compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

Provided herein is a pharmaceutical composition comprising at least one opioid receptor antagonist prodrug compound, or a stereoisomer, pharmaceutically acceptable salt, hydrate, solvate, or N-oxide thereof, together with one or more pharmaceutically acceptable carriers. The carrier(s) (or excipient(s)) is acceptable or suitable if the carrier is compatible with the other ingredients of the composition and not deleterious to the recipient (i.e., the subject) of the composition.

One embodiment provides a pharmaceutical composition comprising a pharmaceutically acceptable excipient and a Disclosed Compound, or a pharmaceutically acceptable salt thereof.

In certain embodiments, the opioid receptor antagonist prodrug compound, i.e., the Disclosed Compound, is substantially pure, in that it contains less than about 5%, or less than about 1%, or less than about 0.1%, of other organic small molecules, such as unreacted intermediates or synthesis by-products that are created, for example, in one or more of the steps of a synthesis method.

Suitable oral dosage forms include, for example, tablets, pills, sachets, or capsules of hard or soft gelatin, methylcellulose or of another suitable material easily dissolved in the digestive tract. In some embodiments, suitable nontoxic solid carriers are used which include, for example, pharmaceutical grades of mannitol, lactose, starch, magnesium stearate, sodium saccharin, talcum, cellulose, glucose, sucrose, magnesium carbonate, and the like. (See, e.g., Remington: The Science and Practice of Pharmacy (Gennaro, 21^{st} Ed. Mack Pub. Co., Easton, PA (2005)).

In some embodiments, the opioid receptor antagonist prodrug compound, i.e., the Disclosed Compound, is formulated for administration by injection. In some instances, the injection formulation is an aqueous formulation. In some instances, the injection formulation is a non-aqueous formulation. In some instances, the injection formulation is an oil-based formulation, such as sesame oil, cottonseed oil, or the like.

The dose of the composition comprising at least one opioid receptor antagonist prodrug compound as described herein differ, depending upon the patient's (e.g., human) condition, that is, general health status, age, and other factors.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

### Dosing and Therapeutic Regimens

In some embodiments, the pharmaceutical compositions described herein are administered for therapeutic applications. In some embodiments, the pharmaceutical composition is administered once per day, twice per day, three times per day, four times per day or more. The pharmaceutical composition is administered daily, every day, every alternate day, two days a week, three days a week, four days a week, five days a week, once a week, every other week, two weeks per month, three weeks per month, once a month, twice a month, three times per month, or other greater or lesser intervening frequency; also, it could be dosed once every 2 months, once every 3 months, once every 4 months, once every 5 months, once every 6 months, once yearly, or with greater or lesser than aforementioned interval frequency. The pharmaceutical composition is administered for at least 1 week, 2 weeks, 1 month, 2 months, 3 months, 4 months, 5 months, 6 months, 7 months, 8 months, 9 months, 10 months, 11 months, 12 months, 18 months, 2 years, 3 years, or more.

In the case wherein the patient's status does not improve, upon the physician's discretion the administration of the composition is given continuously; alternatively, the dose of the composition being administered is temporarily reduced or temporarily suspended for a certain length of time (i.e., a “drug holiday”). In some instances, the length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, 365 days, or 366 days. The dose reduction during a drug holiday is from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, can be adjusted, as a function of the symptoms, to a level at which the improved disease, disorder or condition is retained.

In some embodiments, the amount of given opioid receptor antagonist prodrug compound varies depending upon factors such as the particular compound, the severity of the disease, the identity (e.g., weight) of the subject or host in need of treatment, but nevertheless is routinely determined in a manner known in the art according to the particular circumstances surrounding the case, including, e.g., the specific agent being administered, the route of administration, and the subject or host being treated. In some instances, the desired dose is conveniently presented in a single dose or as divided doses administered simultaneously (or over a short period of time) or at appropriate intervals, for example as two, three, four or more sub-doses per day.

In some embodiments, the amount of given opioid receptor antagonist prodrug compound will typically be in the range of about 0.02 mg to about 5000 mg per dose. (Note: all prodrug mass quantities are expressed in base moiety equivalents). In some embodiments, the amount of given opioid receptor antagonist prodrug compound is in the range of about 1 mg to about 5000 mg per dose. In some embodiments, the amount of given opioid receptor antagonist prodrug compound is in the range of about 10 mg to about 1600 mg per dose. The desired dose may conveniently be presented in a single dose or as divided doses administered simultaneously (or over a short period of time) or at appropriate intervals, for example as two, three, four or more sub-doses per day.

In some embodiments, the daily dosages appropriate for the opioid receptor antagonist prodrug compound described herein are from about 0.01 mg/kg to about 30 mg/kg. In one embodiment, the daily dosages are from about 0.1 mg/kg to about 165 mg/kg. An indicated daily dosage in the larger mammal, including, but not limited to, humans, is in the range from about 0.5 mg to about 1000 mg, conveniently administered in a single dose or in divided doses. Suitable unit dosage forms for intramuscular administration include from about 1 to about 5000 mg active ingredient. In one embodiment, the unit dosage is about 10 mg, about 50 mg, about, 100 mg, about 200 mg, about 500 mg, about 1000 mg, about 2000 mg, about 2500 mg, about 4000 mg, or about 5000 mg.

The foregoing ranges are merely suggestive, as the number of variables in regard to an individual treatment regime is large, and considerable excursions from these recommended values are not uncommon. Such dosages may be altered depending on a number of variables, not limited to the activity of the compound used, the disease or condition to be treated, the mode of administration, the requirements of the individual subject, the severity of the disease or condition being treated, and the judgment of the practitioner.

### Methods of Treatment

In some embodiments, described herein is a method of treating one or more medical conditions in a subject in need thereof, comprising administering to the subject in need thereof an opioid receptor antagonist compound described herein.

In some embodiments, the medical condition is selected from the group comprising opioid dependence, alcohol dependence, drug addiction, polydrug addiction and pain.

In some embodiments, the medical condition is a depressive disorder.

In some embodiments, the medical condition involves side effects brought by opioid use, wherein the side effects include itchiness and opioid-induced constipation.

In some embodiments, the medical condition is congenital insensitivity to pain with anhidrosis.

In some embodiments, the medical condition is abnormally lowered blood pressure in a patient experiencing septic shock.

In some embodiments, described herein is an opioid receptor antagonist compound for use in reduction of opioid consumption in a patient with opioid dependence.

In some embodiments, described herein is an opioid receptor antagonist compound for use in reduction of alcohol consumption in a patient with alcohol dependence, pathological gambling shopping addiction or other diseases of compulsive behavior.

In some embodiments, described herein is an opioid receptor antagonist compound for use in treating a patient with a depressive disorder.

The depressive disorder is selected from major depressive disorder, chronic depression, severe unipolar recurrent major depressive episodes, dysthymic disorder, depressive neurosis and neurotic depression, melancholic depression, atypical depression, reactive depression, treatment resistant depression, seasonal affective disorder and pediatric depression; premenstrual syndrome, premenstrual dysphoric disorder, hot flashes, bipolar disorders or manic depression, bipolar I disorder, bipolar II disorder and cyclothymic disorder.

In some embodiments, described herein is an opioid receptor antagonist compound for use in treating a patient with side effects brought by opioid use, wherein the side effects include itchiness and opioid-induced constipation.

In some embodiments, described herein is an opioid receptor antagonist compound for use in treating a patient with congenital insensitivity to pain with anhidrosis.

In some embodiments, described herein is an opioid receptor antagonist compound for use in increasing blood pressure in a patient experiencing septic shock.

Provided herein is a method of treating opioid dependence in a patient in need thereof comprising administering a pharmaceutical composition comprising a Disclosed Compound, or pharmaceutically acceptable salt thereof, and a pharmaceutically acceptable excipient. Provided herein is the method wherein the pharmaceutical composition is administered orally. Provided herein is the method wherein the pharmaceutical composition is administered by injection. Provided herein is the method wherein the pharmaceutical composition is administered by intramuscular injection. Provided herein is the method wherein the intramuscular injection is a depot injection. Provided herein is the method wherein the depot injection provides a therapeutically effective concentration for a period of 2 days to 3 months. Provided herein is the method wherein the depot injection provides a therapeutically effective concentration for a period of about 2 days. Provided herein is the method wherein the depot injection provides a therapeutically effective concentration for a period of about 4 days. Provided herein is the method wherein the depot injection provides a therapeutically effective concentration for a period of about 7 days. Provided herein is the method wherein the depot injection provides a therapeutically effective concentration for a period of about 10 days. Provided herein is the method wherein the depot injection provides a therapeutically effective concentration for a period of about 1 week. Provided herein is the method wherein the depot injection provides a therapeutically effective concentration for a period of about 2 weeks. Provided herein is the method wherein the depot injection provides a therapeutically effective concentration for a period of about 3 weeks. Provided herein is the method wherein the depot injection provides a therapeutically effective concentration for a period of about 4 weeks. Provided herein is the method wherein the depot injection provides a therapeutically effective concentration for a period of about 5 weeks. Provided herein is the method wherein the depot injection provides a therapeutically effective concentration for a period of about 6 weeks. Provided herein is the method wherein the depot injection provides a therapeutically effective concentration for a period of about 1 month. Provided herein is the method wherein the depot injection provides a therapeutically effective concentration for a period of about 2 months. Provided herein is the method wherein the depot injection provides a therapeutically effective concentration for a period of about 3 months. Provided herein is the method wherein the depot injection provides a therapeutically effective concentration for a period of about 4 months. Provided herein is the method wherein the depot injection provides a therapeutically effective concentration for a period of about 5 months. Provided herein is the method wherein the depot injection provides a therapeutically effective concentration for a period of about 6 months or greater.

The invention is illustrated by the following examples, which are not intended to be limiting the invention in any way.

### EXEMPLIFICATION

Prodrugs of opioid receptor antagonists such as samidorphan, methocinnamox, and naloxone disclosed herein can be synthesized according to the following general methods.

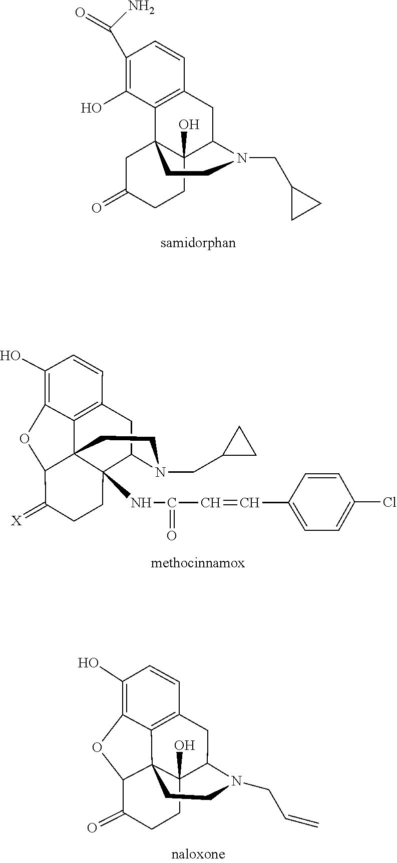

### Abbreviations

| Acronym | Full Name |
|---|---|
| DMF | dimethyl formamide |
| TEA | triethylamine |
| DCM | dichloromethane |
| ACN | acetonitrile |
| EtOAc | ethyl acetate |
| NaI | sodium iodide |
| H₂O | water |
| Et₃N | triethylamine |
| eq | equivalence |
| r.t. | room temperature |
| h | hour |

### General Synthetic Schemes

### Example 1: General Schemes for Synthesizing Samidorphan Prodrugs

The samidorphan prodrug compounds of Formula (I) will be synthesized according to the following four schemes.

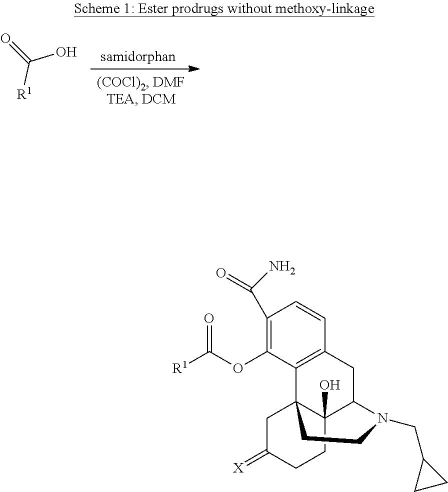

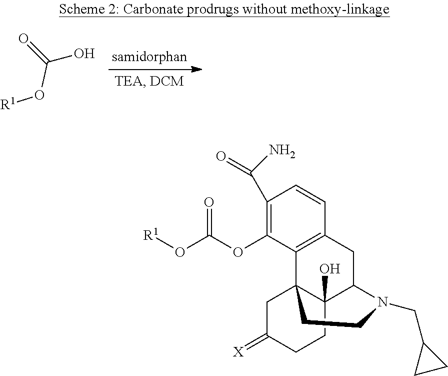

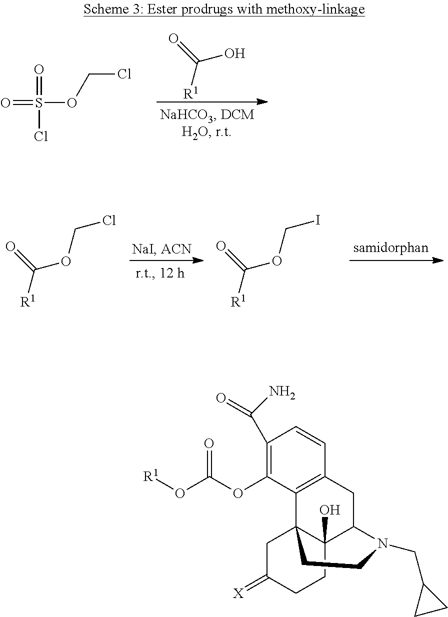

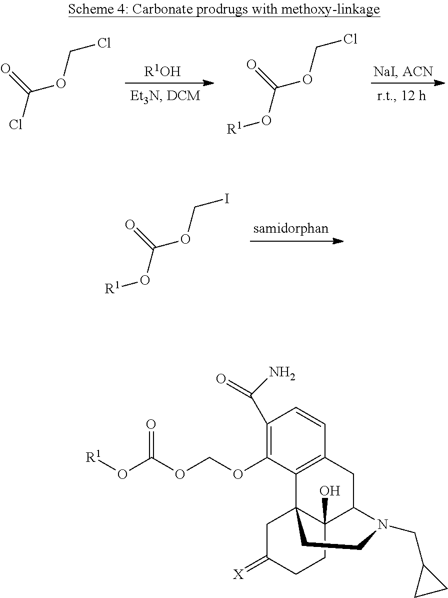

### Example 2: General Schemes for Synthesizing Methocinnamox Prodrugs

The methocinnamox prodrug compounds of Formula (II) will be synthesized according to the following four schemes.

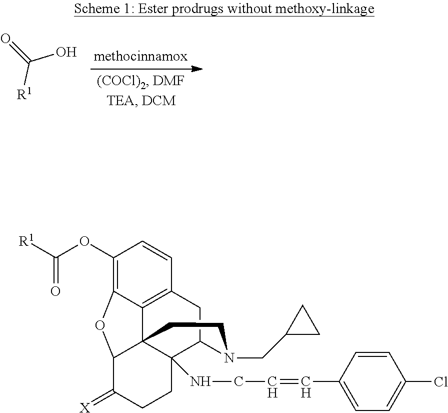

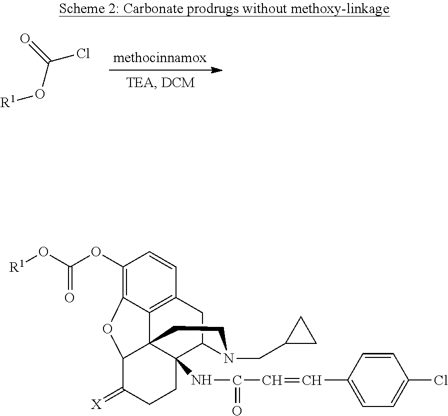

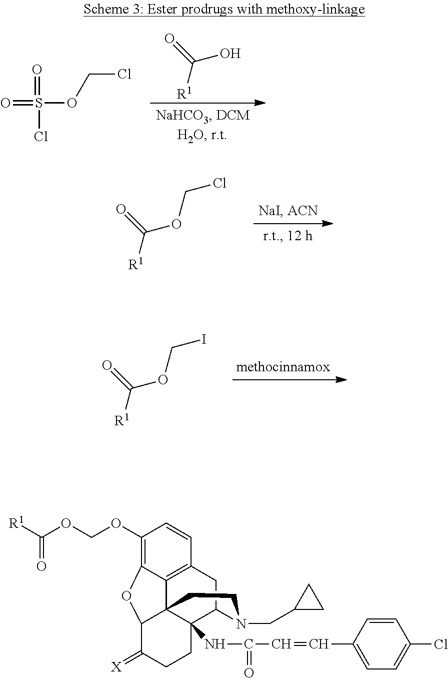

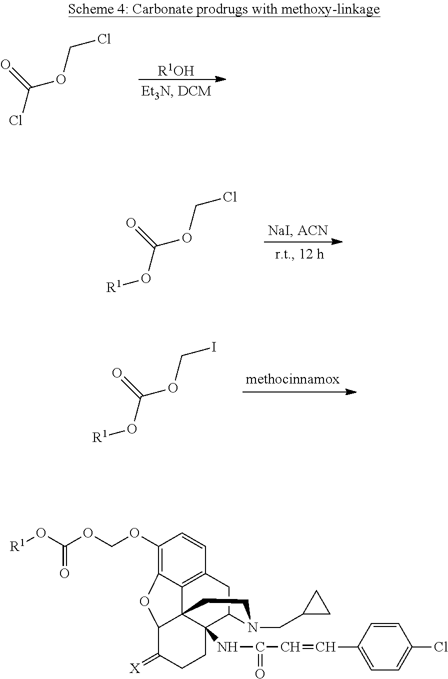

### Example 3: General Schemes for Synthesizing Naloxone Prodrugs

The naloxone prodrug compounds of Formula (III) will be synthesized according to the following four schemes.

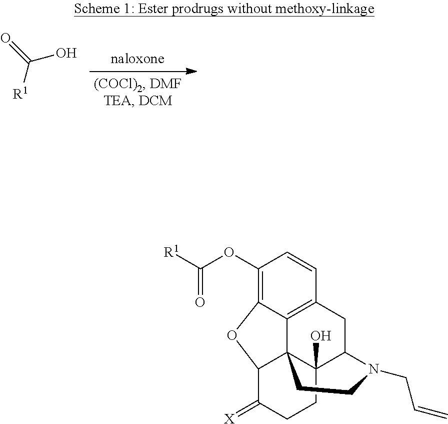

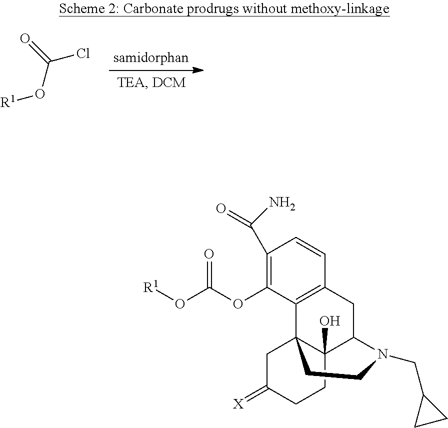

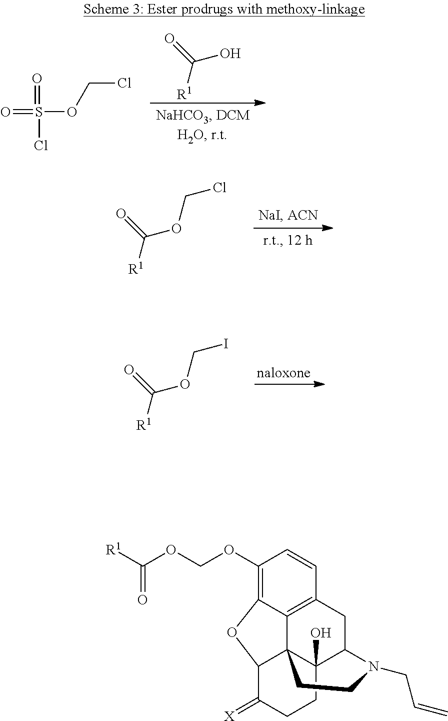

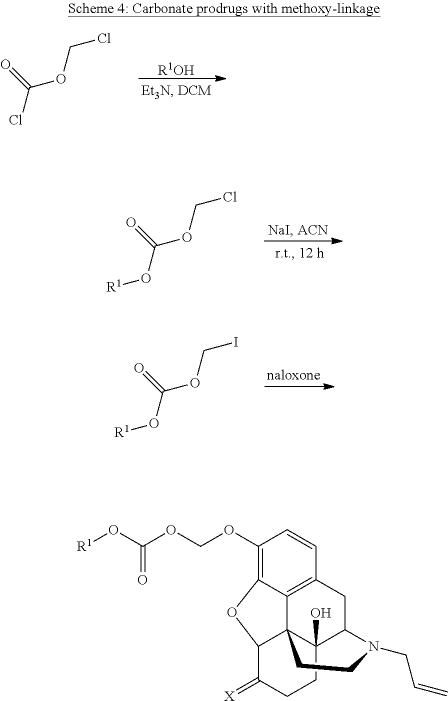

### Preparation of Prodrug Compounds

### Example 4: [(3R,4aS,7aR,12bS)-3-allyl-4a-hydroxy-7-oxo-2,4,5,6,7a,13-hexahydro-1H-4,12-methanobenzofuro[3,2-e]isoquinoline-9-yl]oxymethyl hexanoate (III-1, Naloxone Prodrug)

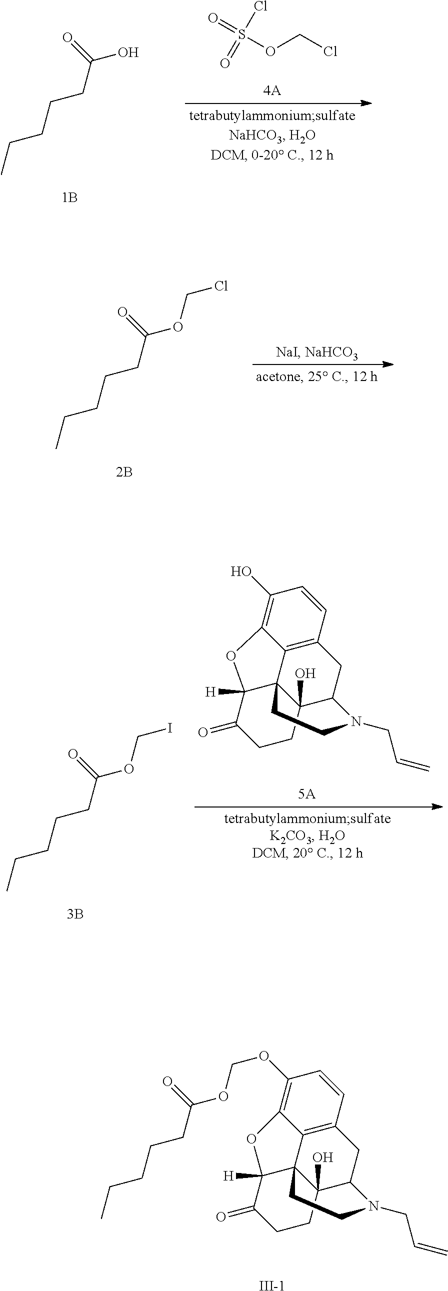

### Step 1: Preparation of Chloromethyl Hexanoate (Compound 2B):

NaHCO₃ (202.50 g, 2.41 mol, 93.75 mL, 4 eq) was added to a solution of hexanoic acid (70 g, 602.63 mmol, 75.51 mL, 1 eq) and tetrabutylammonium sulfate (70.02 g, 60.26 mmol, 69.33 mL, 50% purity, 0.1 eq) in DCM (500 mL) and H₂O (500 mL). The reaction mixture was added with chloro(chlorosulfonyloxy)methane (99.43 g, 602.63 mmol, 1 eq) in DCM (100 mL) at 0° C. The mixture was stirred at 20° C. for 12 h under N₂. The mixture was diluted with H₂O (1500 mL) and the aqueous phase was extracted twice with DCM (1500 mL). The combined organic phases were washed twice with brine (500 mL), dried with anhydrous Na₂SO₄, filtered and concentrated under reduced pressure to give a residue. The residue was purified by column chromatography (SiO₂, Petroleum ether/Ethyl acetate=100/1 to 50/1). Compound chloromethyl hexanoate (40 g, 242.97 mmol, 40.32% yield) was obtained as colorless oil.

### Step 2: Preparation of Iodomethyl Hexanoate (Compound 3B):

A mixture of chloromethyl hexanoate (35 g, 212.60 mmol, 1 eq), NaI (127.47 g, 850.39 mmol, 4 eq), and NaHCO₃ (71.44 g, 850.39 mmol, 33.07 mL, 4 eq) in acetone (1000 mL) was degassed and purged with N₂ for 3 times, and then the mixture was stirred at 25° C. for 12 h under N₂ atmosphere in the dark. The reaction mixture was concentrated under reduced pressure to give a residue. The residue was diluted with H₂O (1000 mL) and the aqueous phase was extracted twice with EtOAc (500 mL). The combined organic phases were washed twice with brine (200 mL), dried with anhydrous Na₂SO₄, filtered and concentrated under reduced pressure to give a residue. The crude product was used in the next step without further purification. Compound iodomethyl hexanoate (30 g, crude) was obtained as black brown oil.

### Step 3: Preparation of the Prodrug Compound III-1:

A mixture of (4R,4aS,7aR,12bS)-3-allyl-4a,9-dihydroxy-2,4,5,6,7a,13-hexahydro-1H-4,12-methanobenzofuro [3,2-e]isoquinoline-7-one dihydrate hydrochloride (23 g, 57.52 mmol, 1 eq) and K₂CO₃ (23.85 g, 172.56 mmol, 3 eq) in H₂O (300 mL) was stirred at 20° C. for 30 min, and then the reaction mixture was added with tetrabutylammonium sulfate (66.84 g, 57.52 mmol, 66.17 mL, 50% purity, 1 eq) and DCM (150 mL) at 20° C. The reaction mixture was stirred at 20° C. for 30 min and a solution of iodomethyl hexanoate (22.09 g, 86.28 mmol, 1.5 eq) in DCM (150 mL) was added to the mixture and degassed and purged with N₂ for 3 times, and then the mixture was stirred at 20° C. for 11 h under N₂ atmosphere. The mixture was diluted with H₂O (2000 mL) and the aqueous phase was extracted twice with EtOAc (1000 mL). The combined organic phases were washed twice with brine (500 mL), dried with anhydrous Na₂SO₄, filtered and concentrated under reduced pressure to give a residue. The residue was purified by column chromatography (SiO₂, Petroleum ether/Ethyl acetate=10/1 to 1/1) to obtain 25 g of the residue. The residue was purified by p-HPLC (column: Xtimate C18 10 u 250 mm by 80 mm; mobile phase: [water (10 mM NH₄HCO₃)-ACN]; B %: 65%-85%, 25 min). Compound [(3R,4aS,7aR,12bS)-3-allyl-4a-hydroxy-7-oxo-2,4,5,6,7a,13-hexahydro-1H-4,12-methanobenzofuro[3,2-e]isoquinoline-9-yl]oxymethyl hexanoate (11 g, 24.03 mmol, 41.77% yield, 99.5% purity) was obtained as a light yellow oil.

¹H NMR (CDCl₃) δ 6.81 (d, J=8.3 Hz, 1H), 6.63 (d, J=8.2 Hz, 1H), 5.91 (d, J=6.0 Hz, 1H), 5.88-5.82 (m, 1H), 5.79 (d, J=6.0 Hz, 1H), 5.26-5.15 (m, 2H), 4.95 (br, 1H), 4.69 (s, 1H), 3.21-2.96 (m, 5H), 2.62-2.55 (m, 2H), 2.44-2.29 (m, 4H), 2.17-2.10 (m, 1H), 1.90-1.86 (m, 1H), 1.66-1.55 (m, 4H), 1.30-1.27 (m, 4H), 0.87 (t, J=6.4 Hz, 3H). LCMS: m/z (MS+H⁺)=456.3.

### Example 5: [(3R,4aS,7aR,12bS)-3-allyl-4a-hydroxy-7-oxo-2,4,5,6,7a,13-hexahydro-1H-4,12-methanobenzofuro[3,2-e]isoquinoline-9-yl]oxymethyl hexanoate (III-2, Naloxone Prodrug)

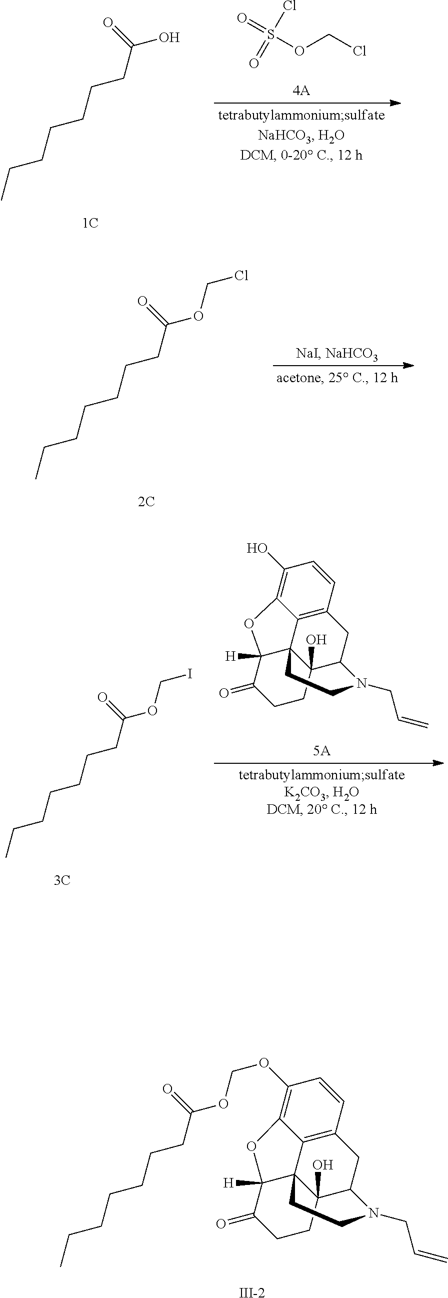

### Step 1: Preparation of Chloromethyl Octanoate (Compound 2C):

NaHCO₃ (163.11 g, 1.94 mol, 75.52 mL, 4 eq) was added to a solution of octanoic acid (70 g, 485.40 mmol, 76.92 mL, 1 eq) and tetrabutylammonium sulfate (56.40 g, 48.54 mmol, 55.84 mL, 50% purity, 0.1 eq) in DCM (500 mL) and H₂O (500 mL). The reaction mixture was added with chloro(chlorosulfonyloxy)methane (80.09 g, 485.40 mmol, 1 eq) in DCM (100 mL) at 0° C. The mixture was stirred at 20° C. for 12 h under N₂. The mixture was diluted with H₂O (1500 mL) and the aqueous phase was extracted twice with DCM (1500 mL). The combined organic phases were washed twice with brine (500 mL), dried with anhydrous Na₂SO₄, filtered and concentrated under reduced pressure to give a residue. The residue was purified by column chromatography (SiO₂, Petroleum ether/Ethyl acetate=100/1 to 50/1). Compound chloromethyl octanoate (40 g, 207.60 mmol, 42.77% yield) was obtained as colorless oil.

### Step 2: Preparation of Iodomethyl Octanoate (Compound 3C):

A mixture of chloromethyl octanoate (35 g, 181.65 mmol, 1 eq), NaI (108.91 g, 726.58 mmol, 4 eq), and NaHCO₃ (61.04 g, 726.58 mmol, 28.26 mL, 4 eq) in acetone (1000 mL) was degassed and purged with N₂ for 3 times, and then the mixture was stirred at 25° C. for 12 hr under N₂ atmosphere in the dark. The reaction mixture was concentrated under reduced pressure to give a residue. The residue was diluted with H₂O (1000 mL) and the aqueous phase was extracted twice with EtOAc (500 mL). The combined organic phases were washed twice with brine (200 mL), dried with anhydrous Na₂SO₄, filtered and concentrated under reduced pressure to give a residue. The crude product was used in the next step without further purification. Compound iodomethyl octanoate (30 g, crude) was obtained as black brown oil.

### Step 3: Preparation of the Prodrug Compound III-2:

A mixture of (4R,4aS,7aR,12bS)-3-allyl-4a,9-dihydroxy-2,4,5,6,7a,13-hexahydro-1H-4,12-methanobenzofuro [3,2-e]isoquinoline-7-one dihydrate hydrochloride (23 g, 57.52 mmol, 1 eq) and K₂CO₃ (23.85 g, 172.56 mmol, 3 eq) in H₂O (300 mL) was stirred at 20° C. for 30 min, and then the reaction mixture was added with tetrabutylammonium sulfate (66.84 g, 57.52 mmol, 66.17 mL, 50% purity, 1 eq) and DCM (150 mL) at 20° C. The reaction mixture was stirred at 20° C. for 30 min and a solution of iodomethyl hexanoate (22.09 g, 86.28 mmol, 1.5 eq) in DCM (150 mL) was added to the mixture and degassed and purged with N₂ for 3 times, and then the mixture was stirred at 20° C. for 11 h under N₂ atmosphere. The mixture was diluted with H₂O (2000 mL) and the aqueous phase was extracted twice with EtOAc (1000 mL). The combined organic phases were washed twice with brine (500 mL), dried with anhydrous Na₂SO₄, filtered and concentrated under reduced pressure to give a residue. The residue was purified by column chromatography (SiO₂, Petroleum ether/Ethyl acetate=10/1 to 1/1) to obtain 25 g of the residue. The residue was purified by p-HPLC (column: Xtimate C18 10 u 250 mm by 80 mm; mobile phase: [water (10 mM NH₄HCO₃)-ACN]; B %: 65%-85%, 25 min). Compound [(3R,4aS,7aR,12bS)-3-allyl-4a-hydroxy-7-oxo-2,4,5,6,7a,13-hexahydro-1H-4,12-methanobenzofuro[3,2-e]isoquinoline-9-yl]oxymethyl hexanoate (11 g, 24.03 mmol, 41.77% yield, 99.5% purity) was obtained as a light yellow oil.

¹H NMR (CDCl₃) δ 6.81 (d, J=8.0 Hz, 1H), 6.63 (d, J=8.4 Hz, 1H), 5.91 (d, J=6.0 Hz, 1H), 5.88-5.82 (m, 1H), 5.79 (d, J=6.0 Hz, 1H), 5.25-5.15 (m, 2H), 4.99 (br, 1H), 4.69 (s, 1H), 3.21-2.96 (m, 5H), 2.62-2.55 (m, 2H), 2.44-2.29 (m, 4H), 2.17-2.10 (m, 1H), 1.90-1.86 (m, 1H), 1.66-1.55 (m, 4H), 1.40-1.26 (m, 8H), 0.87 (t, J=6.8 Hz, 3H). LCMS: m/z (MS+H⁺)=484.3.

### Example 6: [(3R,4aS,7aR,12bS)-3-(cyclopropylmethyl)-4a-hydroxy-7-oxo-2,4,5,6,7a,13-hexahydro-1H-4,12-methanobenzofuro[3,2-e]isoquinoline-9-yl]oxymethyl butanoate (IV-1, Naltrexone Prodrug)

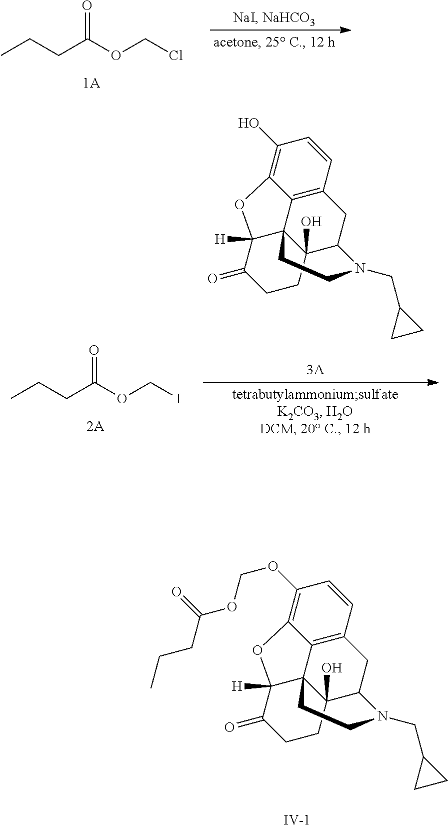

### Step 1: Preparation of Iodomethyl Butanoate (Compound 2A):

A mixture of chloromethyl butanoate (40 g, 292.88 mmol, 1 eq), NaI (175.60 g, 1.17 mol, 4 eq), and NaHCO₃ (98.42 g, 1.17 mol, 4 eq) in acetone (1500 mL) was degassed and purged with N₂ for 3 times, and then the mixture was stirred at 25° C. for 12 h under N₂ atmosphere in the dark. The reaction mixture was concentrated under reduced pressure to give a residue. The residue was diluted with H₂O (3000 mL) and the aqueous phase was extracted twice with EtOAc (2000 mL). The combined organic phases were washed twice with brine (500 mL), dried with anhydrous Na₂SO₄, filtered and concentrated under reduced pressure to give a residue. The crude product was used in the next step without further purification. Compound iodomethyl butanoate (30 g, crude) was obtained as black brown oil.

### Step 2: Preparation of the Prodrug Compound IV-1:

A mixture of (3R,4aS,7aR,12bS)-3-(cyclopropylmethyl)-4a,9-dihydroxy-2,4,5,6,7a,13-hexahydro-1H-4,12-methanobenzofuro[3,2-e]isoquinoline-7-one (30 g, 79.39 mmol, 1 eq, HCl) and K₂CO₃ (32.92 g, 238.18 mmol, 3 eq) in H₂O (300 mL) was stirred at 20° C. for 30 min, and then to the reaction mixture was added with tetrabutylammonium sulfate (92.25 g, 79.39 mmol, 91.34 mL, 50% purity, 1 eq) and DCM (150 mL) at 20° C. The reaction mixture was stirred at 20° C. for 30 min, and a solution of iodomethyl butanoate (27.16 g, 119.09 mmol, 1.5 eq) in DCM (150 mL) was added to the mixture and it was degassed and purged with N₂ for 3 times. The mixture was stirred at 20° C. for 11 h under N₂ atmosphere. The mixture was diluted with H₂O (2000 mL) and the aqueous phase was extracted twice with DCM (1000 mL). The combined organic phases were washed twice with brine (500 mL), dried with anhydrous Na₂SO₄, filtered and concentrated under reduced pressure to give a residue. The residue was purified by column chromatography (SiO₂, Petroleum ether/Ethyl acetate=0/1 to 1/1) to obtain 32 g as crude product. The residue was re-purified by prep-HPLC (column: Phenomenex luna C18 250 mm by 100 mm by 10 um; mobile phase: [water (0.1% TFA)-ACN]; B %: 35%-85%, 10 min) to obtain 19 g product. Next, the product was re-purified by column chromatography (SiO₂, Petroleum ether/Ethyl acetate=0/1 to 1/1). Compound [(3R,4aS,7aR,12bS)-3-(cyclopropylmethyl)-4a-hydroxy-7-oxo-2,4,5,6,7a,13-hexahydro-1H-4,12-methanobenzofuro[3,2-e]isoquinoline-9-yl]oxymethyl butanoate (12 g, 26.61 mmol, 33.92% yield, 99.0% purity) was obtained as a light yellow oil.

¹H NMR (CDCl₃) δ 6.81 (d, J=8 Hz, 1H), 6.62 (d, J=8.4 Hz, 1H), 5.92 (d, J=6 Hz, 1H), 5.80 (d, J=6 Hz, 1H), 5.19 (br, 1H), 4.70 (s, 1H), 3.19 (d, J=6 Hz 1H), 3.09-2.99 (m, 2H), 2.74-2.68 (m, 1H), 2.62-2.55 (m, 1H), 2.47-2.29 (m, 6H), 2.17-2.10 (m, 1H), 1.93-1.87 (m, 1H), 1.72-1.55 (m, 5H, may be mixed with water peak), 0.95-0.84 (m, 4H), 0.550.59 (m, 2H), 0.18-0.14 (m, 2H). LCMS: m/z (MS+H⁺)=422.2.

### Example 7: [(3R,4aS,7aR,12bS)-3-(cyclopropylmethyl)-4a-hydroxy-7-oxo-2,4,5,6,7a,13-hexahydro-1H-4,12-methanobenzofuro[3,2-e]isoquinoline-9-yl]oxymethyl hexanoate (IV-2, Naltrexone Prodrug)

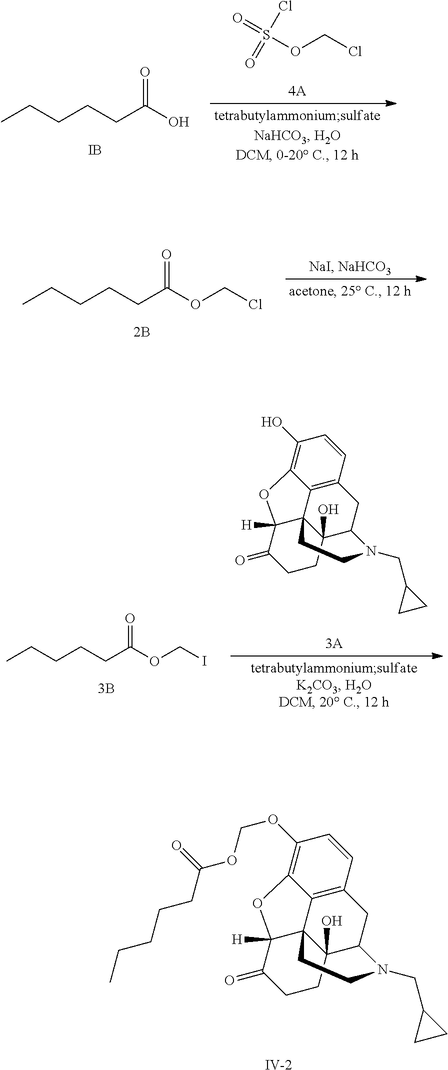

### Step 1: Preparation of Chloromethyl Hexanoate (Compound 2B):

NaHCO₃ (202.50 g, 2.42 mol, 93.76 mL, 4 eq) was added to a solution of hexanoic acid (70 g, 301.31 mmol, 37.76 mL, 1 eq) and tetrabutylammonium sulfate (70.02 g, 60.26 mmol, 69.34 mL, 50% purity, 0.1 eq) in DCM (600 mL) and H₂O (600 mL). The reaction mixture was added with chloro(chlorosulfonyloxy)methane (99.44 g, 602.62 mmol, 1 eq) in DCM (100 mL) at 0° C. The mixture was stirred at 25° C. for 12 h under N₂. The mixture was diluted with H₂O (3000 mL) and the aqueous phase was extracted twice with DCM (3000 mL). The combined organic phases were washed twice with brine (500 mL), dried with anhydrous Na₂SO₄, filtered and concentrated under reduced pressure to give a residue. The residue was purified by column chromatography (SiO₂, Petroleum ether/Ethyl acetate=100/1 to 50/1). Compound chloromethyl hexanoate (45 g, 273.34 mmol, 45.36% yield) was obtained as light yellow oil.

### Step 2: Preparation of Iodomethyl Hexanoate (Compound 3B):

A mixture of chloromethyl hexanoate (45 g, 121.48 mmol, 1 eq), NaI (163.89 g, 1.09 mol, 4 eq), and NaHCO₃ (91.84 g, 1.09 mol, 4 eq) in acetone (1500 mL) was degassed and purged with N₂ for 3 times, and then the mixture was stirred at 25° C. for 12 h under N₂ atmosphere in the dark. The reaction mixture was concentrated under reduced pressure to give a residue. The residue was diluted with H₂O (3000 mL) and the aqueous phase was extracted twice with EtOAc (2000 mL). The combined organic phases were washed twice with brine (500 mL), dried with anhydrous Na₂SO₄, filtered and concentrated under reduced pressure to give a residue. The crude product was used in the next step without further purification. Compound iodomethyl hexanoate (40 g, crude) was obtained as brown oil.

### Step 3: Preparation of the Prodrug Compound IV-2:

A mixture of (3R,4aS,7aR,12bS)-3-(cyclopropylmethyl)-4a,9-dihydroxy-2,4,5,6,7a,13-hexahydro-1H-4,12-methanobenzofuro[3,2-e]isoquinoline-7-one (30 g, 79.39 mmol, 1 eq, HCl) and K₂CO₃ (32.92 g, 238.18 mmol, 3 eq) in H₂O (250 mL) was stirred at 20° C. for 30 min, and then to the reaction mixture was added with tetrabutylammonium sulfate (92.25 g, 79.39 mmol, 91.34 mL, 50% purity, 1 eq) and DCM (125 mL) at 20° C. The reaction mixture was stirred at 20° C. for 30 min. A solution of iodomethyl hexanoate (30.50 g, 119.09 mmol, 1.5 eq) in DCM (125 mL) was added to the mixture and degassed and purged with N₂ for 3 times, and then the mixture was stirred at 20° C. for 11 h under N₂ atmosphere. The mixture was diluted with H₂O (3000 mL) and the aqueous phase was extracted twice with DCM (2000 mL). The combined organic phases were washed twice with brine (500 mL), dried with anhydrous Na₂SO₄, filtered and concentrated under reduced pressure to give a residue. The residue was purified by column chromatography (SiO₂, Petroleum ether/Ethyl acetate=10/1 to 1/1) to obtain 28 g of the residue. The residue was purified by prep-HPLC (column: Phenomenex luna C18 250 mm by 100 mm by 10 um; mobile phase: [water (0.1% TFA)-ACN]; B %: 35%-85%, 10 min) obtain 12 g product. Next, the product was purified by column chromatography (SiO₂, Petroleum ether/Ethyl acetate=50/1 to 1/1). Compound [(3R,4aS,7aR,12bS)-3-(cyclopropylmethyl)-4a-hydroxy-7-oxo-2,4,5,6,7a,13-hexahydro-1H-4,12-methanobenzofuro[3,2-e]isoquinoline-9-yl]oxymethyl hexanoate (11 g, 24.35 mmol, 29.35% yield, 99.5% purity) was obtained as a yellow oil.

¹H NMR (CDCl₃) δ 6.81 (d, J=8.4 Hz, 1H), 6.61 (d, J=8.0 Hz, 1H), 5.92 (d, J=6.0 Hz, 1H), 5.80 (d, J=6.0 Hz, 1H), 5.20 (br, 1H), 4.70 (s, 1H), 3.19 (d, J=5.6 Hz 1H), 3.09-2.99 (m, 2H), 2.73-2.69 (m, 1H), 2.62-2.56 (m, 1H), 2.47-2.29 (m, 6H), 2.17-2.10 (m, 1H), 1.92-1.87 (m, 1H), 1.68-1.55 (m, 5H), 1.32-1.27 (m, 4H), 0.90-0.82 (m, 4H), 0.59-0.54 (m, 2H), 0.18-0.14 (m, 2H). LCMS: m/z (MS+H⁺)=470.3.

### Example 8: [(3R,4aS,7aR,12bS)-3-(cyclopropylmethyl)-4a-hydroxy-7-oxo-2,4,5,6,7a,13-hexahydro-1H-4,12-methanobenzofuro[3,2-e]isoquinoline-9-yl]oxymethyl octanoate (IV-3, Naltrexone Prodrug)

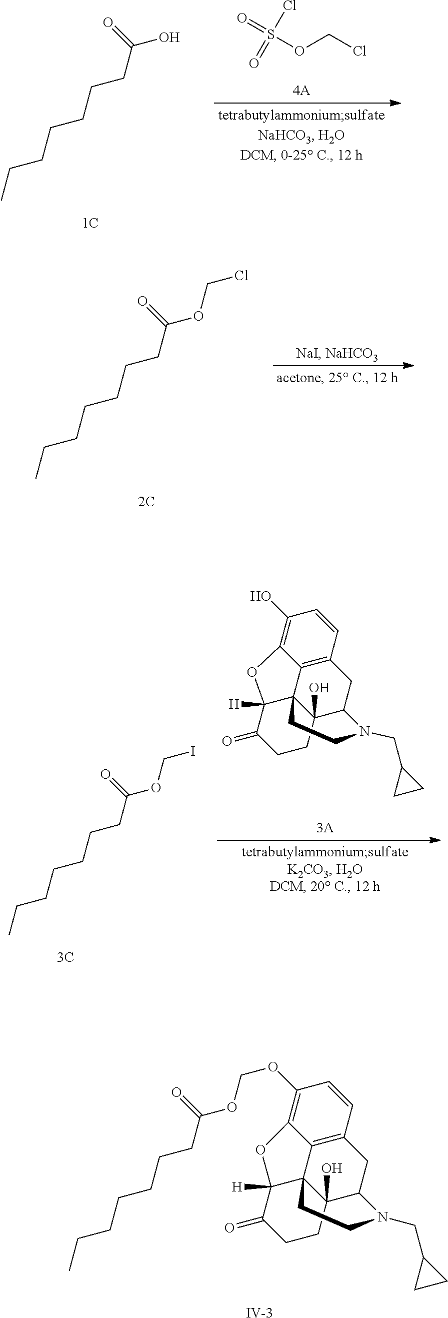

### Step 1: Preparation of Chloromethyl Octanoate (Compound 2C):

NaHCO₃ (116.51 g, 1.39 mol, 53.94 mL, 4 eq) was added to a solution of octanoic acid (50 g, 346.71 mmol, 54.95 mL, 1 eq) and tetrabutylammonium sulfate (40.29 g, 34.67 mmol, 39.89 mL, 50% purity, 0.1 eq) in DCM (300 mL) and H₂O (300 mL). The reaction mixture was added with chloro(chlorosulfonyloxy)methane (57.21 g, 346.71 mmol, 1 eq) in DCM (60 mL) at 0° C. The mixture was stirred at 25° C. for 12 h under N₂. The mixture was diluted with H₂O (3000 mL) and the aqueous phase was extracted twice with DCM (2000 mL). The combined organic phases were washed twice with brine (500 mL), dried with anhydrous Na₂SO₄, filtered and concentrated under reduced pressure to give a residue. The residue was purified by column chromatography (SiO₂, Petroleum ether/Ethyl acetate=100/1 to 50/1). Compound chloromethyl octanoate (45 g, 233.54 mmol, 67.36% yield) was obtained as light yellow oil.

### Step 2: Preparation of Iodomethyl Octanoate (Compound 3C):

A mixture of chloromethyl octanoate (45 g, 103.80 mmol, 1 eq), NaI (140.02 g, 934.18 mmol, 4 eq), and NaHCO₃ (78.48 g, 934.18 mmol, 4 eq) in acetone (1500 mL) was degassed and purged with N₂ for 3 times, and then the mixture was stirred at 25° C. for 12 h under N₂ atmosphere in dark. The reaction mixture was concentrated under reduced pressure to give a residue. The residue was diluted with H₂O (3000 mL) and the aqueous phase was extracted twice with EtOAc (2000 mL). The combined organic phases were washed twice with brine (500 mL), dried with anhydrous Na₂SO₄, filtered and concentrated under reduced pressure to give a residue. The crude product was used in the next step without further purification. Compound iodomethyl octanoate (40 g, crude) was obtained as brown oil.

### Step 3: Preparation of the Prodrug Compound IV-3:

A mixture of (3R,4aS,7aR,12bS)-3-(cyclopropylmethyl)-4a,9-dihydroxy-2,4,5,6,7a,13-hexahydro-1H-4,12-methanobenzofuro[3,2-e]isoquinoline-7-one (30 g, 79.39 mmol, 1 eq, HCl) and K₂CO₃ (32.92 g, 238.18 mmol, 3 eq) in H₂O (250 mL) was stirred at 20° C. for 30 min, and then the reaction mixture was added with tetrabutylammonium sulfate (92.25 g, 79.39 mmol, 91.34 mL, 50% purity, 1 eq) and DCM (125 mL) at 20° C. The reaction mixture was stirred at 20° C. for 30 min, and a solution of iodomethyl octanoate (33.84 g, 119.09 mmol, 1.5 eq) in DCM (125 mL) was added to the mixture and degassed and purged with N₂ for 3 times. Then the mixture was stirred at 20° C. for 11 h under N₂ atmosphere. The mixture was diluted with H₂O (3000 mL) and the aqueous phase was extracted twice with DCM (2000 mL). The combined organic phases were washed twice with brine (500 mL), dried with anhydrous Na₂SO₄, filtered and concentrated under reduced pressure to give a residue. The residue was purified by column chromatography (SiO₂, Petroleum ether/Ethyl acetate=10/1 to 1/1) to obtain 25 g of the residue. The residue was purified by prep-HPLC (column: Phenomenex luna C18 250 mm by 100 mm by 10 um; mobile phase: [water(0.1% TFA)-ACN]; B %: 35%-85%, 10 min) obtain 12 g product. Next, the product was purified by column chromatography (SiO₂, Petroleum ether/Ethyl acetate=50/1 to 1/1). Compound [(3R,4aS,7aR,12bS)-3-(cyclopropylmethyl)-4a-hydroxy-7-oxo-2,4,5,6,7a,13-hexahydro-1H-4,12-methanobenzofuro[3,2-e]isoquinoline-9-yl]oxymethyl octanoate (11 g, 23.39 mmol, 27.59% yield, 99.1% purity) was obtained as a yellow oil.

¹H NMR (CDCl₃) δ 6.81 (d, J=8.0 Hz, 1H), 6.62 (d, J=8.4 Hz, 1H), 5.92 (d, J=6.0 Hz, 1H), 5.80 (d, J=6.0 Hz, 1H), 5.20 (br, 1H), 4.69 (s, 1H), 3.19 (d, J=6 Hz 1H), 3.09-3.00 (m, 2H), 2.73-2.69 (m, 1H), 2.62-2.58 (m, 1H), 2.48-2.29 (m, 6H), 2.17-2.10 (m, 1H), 1.92-1.87 (m, 1H), 1.68-1.56 (m, 6H), 1.32-1.26 (m, 8H), 0.91-0.86 (m, 4H), 0.60-0.55 (m, 2H), 0.18-0.14 (m, 2H). LCMS: m/z (MS+H⁺)=498.3.

### Example 9: [(3R,4aS,7aR,12bS)-3-(cyclopropylmethyl)-4a-hydroxy-7-oxo-2,4,5,6,7a,13-hexahydro-1H-4,12-methanobenzofuro[3,2-e]isoquinoline-9-yl]oxymethyl butyl carbonate (IV-4, Naltrexone Prodrug)

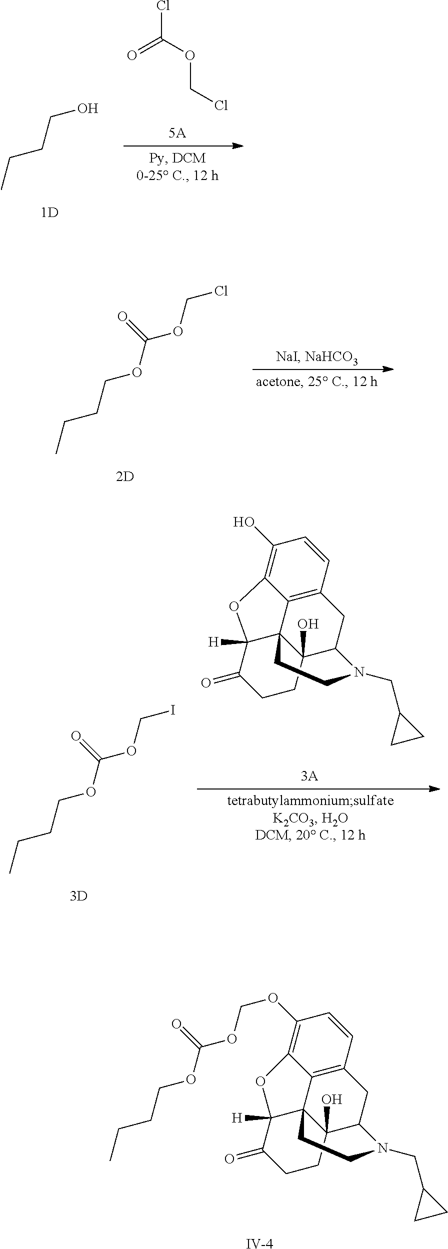

### Step 1: Preparation of Butyl Chloromethyl Carbonate (Compound 2D):

Pyridine (64.04 g, 809.48 mmol, 65.34 mL, 1.5 eq) and chloromethyl carbonochloridate (83.50 g, 647.58 mmol, 57.58 mL, 1.2 eq) were added to a solution of butan-1-ol (40 g, 539.66 mmol, 49.38 mL, 1 eq) in DCM (1000 mL) at 0° C. The mixture was stirred at 25° C. for 12 h under N₂ atmosphere. The mixture was diluted with H₂O (3000 mL) and the aqueous phase was extracted twice with DCM (2000 mL). The combined organic phases were washed twice with brine (500 mL), dried with anhydrous Na₂SO₄, filtered and concentrated under reduced pressure to give a residue. The residue was purified by column chromatography (SiO₂, Petroleum ether/Ethyl acetate=100/1 to 50/1). Compound butyl chloromethyl carbonate (50 g, 300.12 mmol, 55.61% yield) was obtained as light yellow oil.

### Step 2: Preparation of Butyl Iodomethyl Carbonate (Compound 3D):

A mixture of butyl chloromethyl carbonate (50 g, 300.12 mmol, 1 eq), NaI (179.94 g, 1.20 mol, 4 eq), and NaHCO₃ (100.85 g, 1.20 mol, 46.69 mL, 4 eq) in acetone (1500 mL) was degassed and purged with N₂ for 3 times, and then the mixture was stirred at 25° C. for 12 h under N₂ atmosphere in the dark. The reaction mixture was concentrated under reduced pressure to give a residue. The residue was diluted with H₂O (3000 mL) and the aqueous phase was extracted twice with EtOAc (2000 mL). The combined organic phases were washed twice with brine (500 mL), dried with anhydrous Na₂SO₄, filtered and concentrated under reduced pressure to give a residue. The crude product was used in the next step without further purification. Compound butyl iodomethyl carbonate (35 g, crude) was obtained as black brown oil.

### Step 3: Preparation of the Prodrug Compound IV-4:

A mixture of (3R,4aS,7aR,12bS)-3-(cyclopropylmethyl)-4a,9-dihydroxy-2,4,5,6,7a,13-hexahydro-1H-4,12-methanobenzofuro[3,2-e]isoquinoline-7-one (30 g, 79.39 mmol, 1 eq, HCl) and K₂CO₃ (32.92 g, 238.18 mmol, 3 eq) in H₂O (300 mL) was stirred at 20° C. for 30 min, and then the reaction mixture was added with tetrabutylammonium sulfate (92.25 g, 79.39 mmol, 91.34 mL, 50% purity, 1 eq) and DCM (150 mL) at 20° C. The reaction mixture was stirred at 20° C. for 30 min, and a solution of butyl iodomethyl carbonate (30.73 g, 119.09 mmol, 1.5 eq) in DCM (150 mL) was added to the mixture and degassed and purged with N₂ for 3 times. Then the mixture was stirred at 20° C. for 11 hr under N₂ atmosphere. The mixture was diluted with H₂O (3000 mL) and the aqueous phase was extracted twice with DCM (2000 mL). The combined organic phases were washed twice with brine (500 mL), dried with anhydrous Na₂SO₄, filtered and concentrated under reduced pressure to give a residue. The residue was purified by column chromatography (SiO₂, Petroleum ether/Ethyl acetate=0/1 to 1/1) to obtain 30 g of the residue. The residue was purified by prep-HPLC (column: Phenomenex luna c18 250 mm by 100 mm by 10 um; mobile phase: [water(0.1% TFA)-ACN]; B %: 35%-85%, 10 min) obtain 20 g product. Next, the product was re-purified by column chromatography (SiO₂, Petroleum ether/Ethyl acetate=0/1 to 1/1). Compound [(3R,4aS,7aR,12bS)-3-(cyclopropylmethyl)-4a-hydroxy-7-oxo-2,4,5,6,7a,13-hexahydro-1H-4,12-methanobenzofuro[3,2-e]isoquinoline-9-yl]oxymethyl butyl carbonate (14 g, 29.30 mmol, 36.91% yield, 98.7% purity) was obtained as a white solid.

¹H NMR (CDCl₃) δ 6.85 (d, J=8.0 Hz, 1H), 6.62 (d, J=8.4 Hz, 1H), 5.92 (d, J=6.0 Hz, 1H), 5.81 (d, J=6.0 Hz, 1H), 5.21 (br, 1H), 4.70 (s, 1H), 4.19 (t, J=6.8 Hz, 2H), 3.20 (d, J=5.6 Hz 1H), 3.09-2.99 (m, 2H), 2.73-2.69 (m, 1H), 2.62-2.56 (m, 1H), 2.47-2.41 (m, 3H), 2.34-2.29 (m, 1H), 2.17-2.10 (m, 1H), 1.92-1.87 (m, 1H), 1.70-1.56 (m, 5H, may be mixed with water peak), 1.45-1.35 (m, 2H), 0.96-0.86 (m, 4H), 0.60-0.55 (m, 2H), 0.18-0.14 (m, 2H). LCMS: m/z (MS+H⁺)=472.3.

### Example 10: [(3R,4aS,7aR,12bS)-3-(cyclopropylmethyl)-4a-hydroxy-7-oxo-2,4,5,6,7a,13-hexahydro-1H-4,12-methanobenzofuro[3,2-e]isoquinoline-9-yl]oxymethyl hexyl carbonate (IV-5, Naltrexone Prodrug)

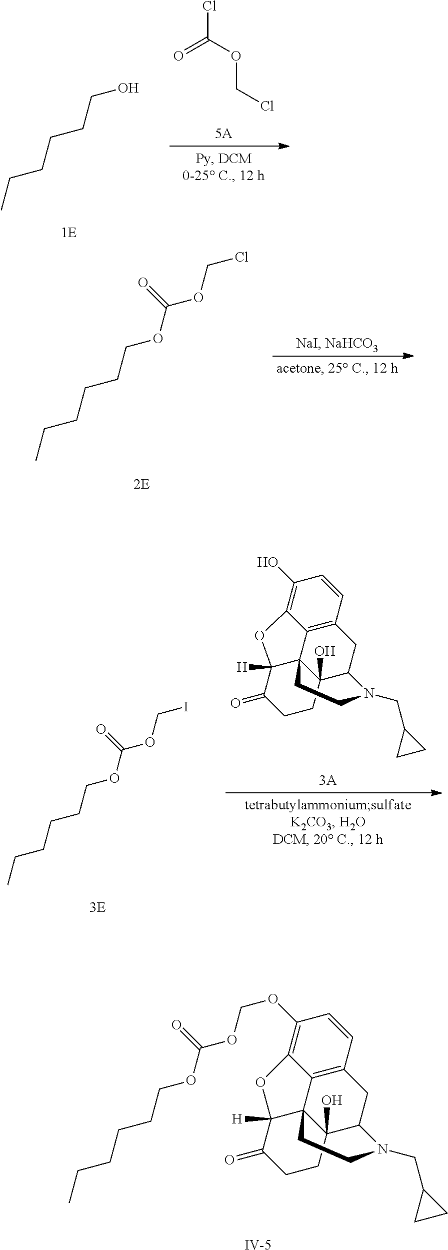

### Step 1: Preparation of Chloromethyl Hexyl Carbonate (Compound 2E):

Pyridine (46.45 g, 587.23 mmol, 47.40 mL, 1.5 eq) and chloromethyl carbonochloridate (60.58 g, 469.79 mmol, 41.78 mL, 1.2 eq) were added to a solution of hexan-1-ol (40 g, 391.49 mmol, 48.78 mL, 1 eq) in DCM (500 mL) at 0° C. The mixture was stirred at 25° C. for 12 h. The mixture was diluted with H₂O (3000 mL) and the aqueous phase was extracted twice with DCM (2000 mL). The combined organic phases were washed twice with brine (500 mL), dried with anhydrous Na₂SO₄, filtered and concentrated under reduced pressure to give a residue. The residue was purified by column chromatography (SiO₂, Petroleum ether/Ethyl acetate=100/1 to 50/1). Compound chloromethyl hexyl carbonate (70 g, 359.61 mmol, 91.86% yield) was obtained as a light yellow oil.

### Step 2: Preparation of Hexyl Iodomethyl Carbonate (Compound 3E):

A mixture of chloromethyl hexyl carbonate (70 g, 359.61 mmol, 1 eq), NaI (323.42 g, 2.16 mol, 6 eq), and NaHCO₃ (181.26 g, 2.16 mol, 83.92 mL, 6 eq) in acetone (2000 mL) was degassed and purged with N₂ for 3 times, and then the mixture was stirred at 25° C. for 12 h under N₂ atmosphere in the dark. The reaction mixture was concentrated under reduced pressure to give a residue. The residue was diluted with H₂O (3000 mL) and the aqueous phase was extracted twice with EtOAc (2000 mL). The combined organic phases were washed twice with brine (500 mL), dried with anhydrous Na₂SO₄, filtered and concentrated under reduced pressure to give a residue. The crude product was used in the next step without further purification. Compound hexyl iodomethyl carbonate (70 g, crude) was obtained as black brown oil.

### Step 3: Preparation of the Prodrug Compound IV-5:

A mixture of (3R,4aS,7aR,12bS)-3-(cyclopropylmethyl)-4a,9-dihydroxy-2,4,5,6,7a,13-hexahydro-1H-4,12-methanobenzofuro[3,2-e]isoquinoline-7-one (30 g, 79.39 mmol, 1 eq, HCl) and K₂CO₃ (32.92 g, 238.18 mmol, 3 eq) in H₂O (300 mL) was stirred at 20° C. for 30 min, and then the reaction mixture was added with tetrabutylammonium, sulfate (92.25 g, 79.39 mmol, 91.34 mL, 50% purity, 1 eq) and DCM (150 mL) at 20° C. The reaction mixture was stirred at 20° C. for 30 min, and a solution of hexyl iodomethyl carbonate (34.07 g, 119.09 mmol, 1.5 eq) in DCM (150 mL) was added to the mixture and degassed and purged with N₂ for 3 times. Then the mixture was stirred at 20° C. for 11 h under N₂ atmosphere. The mixture was diluted with H₂O (3000 mL) and the aqueous phase was extracted twice with DCM (2000 mL). The combined organic phases were washed twice with brine (500 mL), dried with anhydrous Na₂SO₄, filtered and concentrated under reduced pressure to give a residue. The residue was purified by column chromatography (SiO₂, Petroleum ether/Ethyl acetate=0/1 to 1/1) to obtain 37 g. The crude product was purified by prep-HPLC (column: Phenomenex luna C18 250 mm by 100 mm by 10 um; mobile phase: [water(0.1% TFA)-ACN]; B %: 35%-85%, 10 min) obtain 22 g product. Next, the product was purified by column chromatography (SiO₂, Petroleum ether/Ethyl acetate=0/1 to 1/1). Compound [(3R,4aS,7aR,12bS)-3-(cyclopropylmethyl)-4a-hydroxy-7-oxo-2,4,5,6,7a,13-hexahydro-1H-4,12-methanobenzofuro[3,2-e]isoquinoline-9-yl]oxymethyl hexyl carbonate (15 g, 29.51 mmol, 37.17% yield, 98.3% purity) was obtained as a white solid.

¹H NMR (CDCl₃) δ 6.85 (d, J=8.0 Hz, 1H), 6.62 (d, J=8.0 Hz, 1H), 5.92 (d, J=6.0 Hz, 1H), 5.81 (d, J=6.4 Hz, 1H), 5.29 (br, 1H), 4.70 (s, 1H), 4.18 (t, J=6.8 Hz, 2H), 3.19 (d, J=6.0 Hz, 1H), 3.09-2.99 (m, 2H), 2.73-2.69 (m, 1H), 2.62-2.56 (m, 1H), 2.47-2.40 (m, 3H), 2.34-2.29 (m, 1H), 2.17-2.09 (m, 1H), 1.92-1.87 (m, 1H), 1.71-1.56 (m, 5H, may be mixed with water peak), 1.40-1.26 (m, 6H), 0.95-0.83 (m, 4H), 0.59-0.55 (m, 2H), 0.18-0.14 (m, 2H). LCMS: m/z (MS+H⁺)=500.3.

### Example 11: [(3R,4aS,7aR,12bS)-3-(cyclopropylmethyl)-4a-hydroxy-7-oxo-2,4,5,6,7a,13-hexahydro-1H-4,12-methanobenzofuro[3,2-e]isoquinoline-9-yl]oxymethyl octyl carbonate (IV-6, Naltrexone Prodrug)

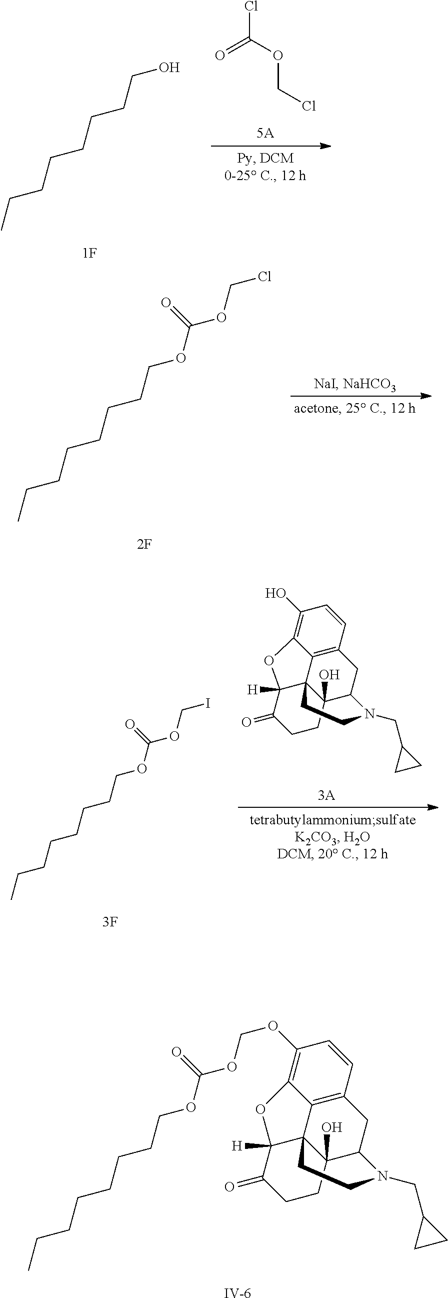

### Step 1: Preparation of Chloromethyl Octyl Carbonate (Compound 2F):

Pyridine (36.44 g, 460.73 mmol, 37.19 mL, 1.5 eq) and chloromethyl carbonochloridate (47.53 g, 368.59 mmol, 32.78 mL, 1.2 eq) were added to a solution of octan-1-ol (40 g, 307.16 mmol, 48.54 mL, 1 eq) in DCM (500 mL) at 0° C. The mixture was stirred at 25° C. for 12 h. The mixture was diluted with H₂O (3000 mL) and the aqueous phase was extracted twice with DCM (2000 mL). The combined organic phases were washed twice with brine (500 mL), dried with anhydrous Na₂SO₄, filtered and concentrated under reduced pressure to give a residue. The residue was purified by column chromatography (SiO₂, Petroleum ether/Ethyl acetate=100/1 to 50/1). Compound chloromethyl octyl carbonate (55 g, 246.96 mmol, 80.40% yield) was obtained as light yellow oil.

### Step 2: Preparation of Iodomethyl Octyl Carbonate (Compound 3F):

A mixture of chloromethyl octyl carbonate (55 g, 246.96 mmol, 1 eq), NaI (222.11 g, 1.48 mol, 6 eq), NaHCO₃ (124.48 g, 1.48 mol, 57.63 mL, 6 eq) in acetone (1500 mL) was degassed and purged with N₂ for 3 times, and then the mixture was stirred at 25° C. for 12 h under N₂ atmosphere in the dark. The reaction mixture was concentrated under reduced pressure to give a residue. The residue was diluted with H₂O (3000 mL) and the aqueous phase was extracted twice with EtOAc (2000 mL). The combined organic phases were washed twice with brine (500 mL), dried with anhydrous Na₂SO₄, filtered and concentrated under reduced pressure to give a residue. The crude product was used in the next step without further purification. Compound iodomethyl octyl carbonate (70 g, crude) was obtained as brown oil.

### Step 3: Preparation of the Prodrug Compound IV-6:

A mixture of (3R,4aS,7aR,12bS)-3-(cyclopropylmethyl)-4a,9-dihydroxy-2,4,5,6,7a,13-hexahydro-1H-4,12-methanobenzofuro[3,2-e]isoquinoline-7-one (30 g, 79.39 mmol, 1 eq, HCl) and K₂CO₃ (32.92 g, 238.18 mmol, 3 eq) in H₂O (300 mL) was stirred at 20° C. for 30 min, and then the reaction mixture was added with tetrabutylammonium sulfate (92.25 g, 79.39 mmol, 91.34 mL, 50% purity, 1 eq) and DCM (150 mL) at 20° C. The reaction mixture was stirred at 20° C. for 30 min, and a solution of iodomethyl octyl carbonate (37.41 g, 119.09 mmol, 1.5 eq) in DCM (150 mL) was added to the mixture and degassed and purged with N₂ for 3 times. Then the mixture was stirred at 20° C. for 11 h under N₂ atmosphere. The mixture was diluted with H₂O (3000 mL) and the aqueous phase was extracted twice with DCM (2000 mL). The combined organic phases were washed twice with brine (500 mL), dried with anhydrous Na₂SO₄, filtered and concentrated under reduced pressure to give a residue. The residue was purified by column chromatography (SiO₂, Petroleum ether/Ethyl acetate=0/1 to 1/1) to obtain 38 g. The residue was purified by prep-HPLC (column: Phenomenex luna C18 250 mm by 100 mm by 10 um; mobile phase: [water(0.1% TFA)-ACN]; B %: 35%-85%, 10 min) obtain 22 g product. Next, the product was purified by column chromatography (SiO₂, Petroleum ether/Ethyl acetate=0/1 to 1/1) obtain 20 g. The residue was purified by prep-HPLC (column: Phenomenex luna C18 250 mm by 100 mm by 10 um; mobile phase: [water(0.1% TFA)-ACN]; B %: 35%-85%, 10 min). Compound [(3R,4aS,7aR,12bS)-3-(cyclopropylmethyl)-4a-hydroxy-7-oxo-2,4,5,6,7a,13-hexahydro-1H-4,12-methanobenzofuro[3,2-e]isoquinoline-9-yl]oxymethyl octyl carbonate (13 g, 24.42 mmol, 30.76% yield, 99.11% purity) was obtained as a light yellow oil.

¹H NMR (CDCl₃) δ 6.85 (d, J=8.0 Hz, 1H), 6.62 (d, J=8.0 Hz, 1H), 5.92 (d, J=6.0 Hz, 11H), 5.81 (d, J=6.1 Hz, 11H), 5.19 (br, 11H), 4.70 (s, 11H), 4.18 (t, J=6.8 Hz, 2H), 3.19 (d, J=5.2 Hz, 11H), 3.09-2.99 (m, 2H), 2.74-2.66 (m, 11H), 2.62-2.56 (m, 11H), 2.47-2.41 (m, 3H), 2.34-2.29 (m, 11H), 2.17-2.10 (m, 11H), 1.92-1.87 (m, 11H), 1.71-1.56 (m, 4H), 1.38-1.28 (m, 10H), 0.93-0.81 (m, 4H), 0.59-0.55 (m, 2H), 0.16-0.15 (m, 2H). LCMS: m/z (MS+H⁺)=528.3.

### Example 12: Solubility of Naloxone and Naltrexone Prodrug Compounds

Table 12 and Table 13 report the solubility data for the naloxone and naltrexone prodrug compounds, respectively.

TABLE 12

| Cmpd ID | Appearance | Activities | Solubility |
|---|---|---|---|
| III-1 | light yellow | solubility study in oil excipients | In cottonseed oil: >50.03 mg/ml |
| | viscous oil | at 50 mg/ml (base equivalents) | In sesame oil: >50.01 mg/ml |
| | | in various oil solvents | In miglyol 812N: <50.53 mg/ml |
| | | lower limit of aqueous solubility | Solubility in water: <0.001 mg/mL |
| III-2 | yellow viscous | solubility study in oil excipients | In cottonseed oil: >50.48 mg/ml |
| | oil | at 50 mg/ml (base equivalents) | In sesame oil: <50.44 mg/ml |
| | | in various oil solvents | In miglyol 812N: <50.28 mg/ml |
| | | lower limit of aqueous solubility | Solubility in water: <0.001 mg/mL |

TABLE 13

| Cmpd ID | Appearance | Activities | Solubility |
|---|---|---|---|
| IV-4 | white solid | solubility study in oil excipients | In cottonseed oil: <50.12 mg/mL |
| | powder | at 50 mg/ml (base equivalents) | In sesame oil: <50.07 mg/mL |
| | | in various oil solvents | In miglyol 812N: <50.41 mg/mL |
| | | lower limit of aqueous solubility | Solubility in water: <0.001 mg/mL |
| IV-5 | white solid | solubility study in oil excipients | In cottonseed oil: <50.19 mg/mL |
| | powder | at 50 mg/ml (base equivalents) | In sesame oil: >50.31 mg/mL |
| | | in various oil solvents | In miglyol 812N: <50.24 mg/mL |
| | | lower limit of aqueous solubility | Solubility in water: <0.001 mg/mL |
| IV-6 | light yellow | solubility study in oil excipients | In cottonseed oil: >50.28 mg/mL |
| | viscous oil | at 50 mg/ml (base equivalents) | In sesame oil: >50.43 mg/mL |
| | | in various oil solvents | In miglyol 812N: >50.11 mg/mL |
| | | lower limit of aqueous solubility | Solubility in water: <0.001 mg/mL |

1. A compound represented by the following structural formula: 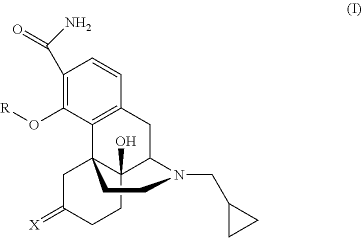
or a pharmaceutically acceptable salt thereof, wherein:
X is O or CH₂;
R is selected from the group consisting of:
(a) —C(O)R¹,
(b) —C(O)OR¹,
(c) —CH₂OC(O)R¹,
(d) —CH₂OC(O)OR¹; OR^{1′} and
(e) —CH₂OR¹;
each instance of R¹ is independently —(CH₂)_{q}CH₃, 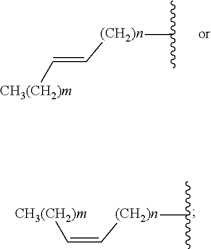
m and n are integers between 0 and 23, and the sum of m and n is an integer between 6 and 23;
and

2. A compound represented by the following structural formula: 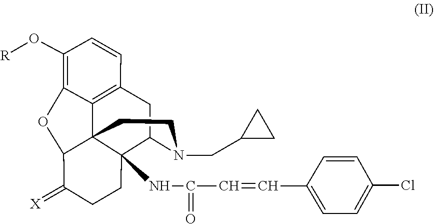
or a pharmaceutically acceptable salt thereof, wherein:
X is O or CH₂;
R is selected from the group consisting of:
(a) —C(O)R¹,
(b) —C(O)OR¹,
(c) —CH₂OC(O)R¹,
(d) —CH₂OC(O)OR^{1′} and
(e); —CH₂OR¹;
each instance of R¹ is independently —(CH₂)_{q}CH₃, 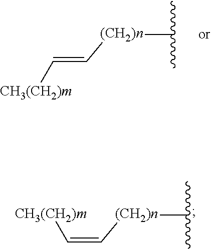
m and n are integers between 0 and 23, and the sum of m and n is an integer from 6 to 23; and
q is an integer between 0 and 25.

3. A compound represented by the following structural formula: 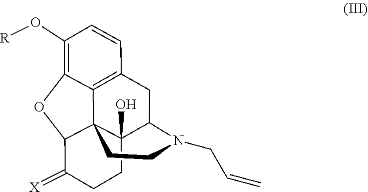
or a pharmaceutically acceptable salt thereof, wherein:
X is O or CH₂;
R is selected from the group consisting of:
(a) —C(O)R¹,
(b) —C(O)OR¹,
(c) —CH₂OC(O)R¹,
(d) —CH₂OC(O)OR¹, and
(e) —CH₂OR¹;
each instance of R¹ is independently —(CH₂)_{q}CH₃, 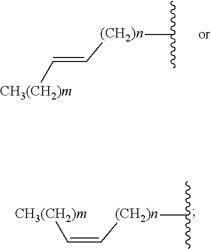
m and n are integers between 0 and 23, and the sum of m and n is an integer from 6 to 23; and
q is an integer between 0 and 25.

4. A pharmaceutical composition comprising a compound of claim 1, or a pharmaceutically acceptable salt thereof, and at least one pharmaceutically acceptable excipient.

5. A method of treating opioid dependence, alcohol dependence, a depressive disorder, itchiness and opioid-induced constipation brought by opioid use, or congenital insensitivity to pain with anhidrosis in a patient in need thereof comprising administering a pharmaceutical composition comprising a compound of claim 1, or pharmaceutically acceptable salt thereof, and a pharmaceutically acceptable excipient.

6. The method of claim 5, wherein the depressive disorder is selected from major depressive disorder, chronic depression, severe unipolar recurrent major depressive episodes, dysthymic disorder, depressive neurosis and neurotic depression, melancholic depression, atypical depression, reactive depression, treatment resistant depression, seasonal affective disorder and pediatric depression; premenstrual syndrome, premenstrual dysphoric disorder, hot flashes, bipolar disorders or manic depression, bipolar I disorder, bipolar II disorder and cyclothymic disorder.

7. A method of increasing blood pressure in a patient experiencing septic shock comprising administering a pharmaceutical composition comprising a compound of claim 1, or pharmaceutically acceptable salt thereof, and a pharmaceutically acceptable excipient.
